SEQUENCE LISTING (288 sequences)

SEQ ID NO: 1 - protein, 5 aa, synthetic construct
TYNMH

SEQ ID NO: 2 - protein, 17 aa, synthetic construct
AIFPGNGHISYAQKFQG

SEQ ID NO: 3 - protein, 10 aa, synthetic construct
GDYDESYFDY

SEQ ID NO: 4 - protein, 11 aa, synthetic construct
KASEGIYNRLA

SEQ ID NO: 5 - protein, 7 aa, synthetic construct
GVISLET

SEQ ID NO: 6 - protein, 9 aa, synthetic construct
QQYWSIPWT

SEQ ID NO: 7 - protein, 8 aa, synthetic construct
GYTFTTYN

SEQ ID NO: 8 - protein, 8 aa, synthetic construct
IFPGNGHI

SEQ ID NO: 9 - protein, 12 aa, synthetic construct
ARGDYDESYFDY

SEQ ID NO: 10 - protein, 6 aa, synthetic construct
EGIYNR

SEQ ID NO: 11 - skipped

SEQ ID NO: 12 - protein, 9 aa, synthetic construct
QQYWSIPWT

SEQ ID NO: 13 - protein, 5 aa, synthetic construct
SYWIS

SEQ ID NO: 14 - protein, 17 aa, synthetic construct
QIFPGDGDINYAQKFQG

SEQ ID NO: 15 - protein, 9 aa, synthetic construct
MDFYSWFPY

SEQ ID NO: 16 - protein, 17 aa, synthetic construct
KSSQSLLNSGNQKNDLA

SEQ ID NO: 17 - protein, 7 aa, synthetic construct
GASTRES

SEQ ID NO: 18 - protein, 9 aa, synthetic construct
QNDHSYPLT

SEQ ID NO: 19 - protein, 8 aa, synthetic construct
GYAFRSYW

SEQ ID NO: 20 - protein, 8 aa, synthetic construct
IFPGDGDI

SEQ ID NO: 21 - protein, 11 aa, synthetic construct
ARMDFYSWFPY

SEQ ID NO: 22 - protein, 12 aa, synthetic construct
QSLLNSGNQKND

SEQ ID NO: 23 - skipped

SEQ ID NO: 24 - protein, 9 aa, synthetic construct
QNDHSYPLT

SEQ ID NO: 25 - protein, 5 aa, synthetic construct
NYYIN

SEQ ID NO: 26 - protein, 16 aa, synthetic construct
FIPMYGTTHYADSVKG

SEQ ID NO: 27 - protein, 10 aa, synthetic construct
GIASMFYPSI

SEQ ID NO: 28 - protein, 11 aa, synthetic construct
QASEDIHGYLA

SEQ ID NO: 29 - protein, 7 aa, synthetic construct
YASTLQS

SEQ ID NO: 30 - protein, 10 aa, synthetic construct
LGDGSTSGIT

SEQ ID NO: 31 - protein, 8 aa, synthetic construct
GFSLSNYY

SEQ ID NO: 32 - protein, 7 aa, synthetic construct
IPMYGTT

SEQ ID NO: 33 - protein, 12 aa, synthetic construct
ARGIASMFYPSI

SEQ ID NO: 34 - protein, 6 aa, synthetic construct
EDIHGY

SEQ ID NO: 35 - skipped

SEQ ID NO: 36 - protein, 10 aa, synthetic construct
LGDGSTSGIT

SEQ ID NO: 37 - protein, 5 aa, synthetic construct
SSTMA

SEQ ID NO: 38 - protein, 16 aa, synthetic construct
IIASSGSAYYAGWAKG

SEQ ID NO: 39 - protein, 11 aa, synthetic construct
QVPGYNDNRYV

SEQ ID NO: 40 - protein, 13 aa, synthetic construct
QASESVYNANSCS

SEQ ID NO: 41 - protein, 7 aa, synthetic construct
DASDLAS

SEQ ID NO: 42 - protein, 10 aa, synthetic construct
AGYKGNGDAA

SEQ ID NO: 43 - protein, 10 aa, synthetic construct
AGYKGNGDAA

SEQ ID NO: 44 - protein, 7 aa, synthetic construct
IASSGSA

SEQ ID NO: 45 - protein, 13 aa, synthetic construct
VRQVPGYNDNRYV

SEQ ID NO: 46 - protein, 8 aa, synthetic construct
ESVYNANS

SEQ ID NO: 47 - skipped

SEQ ID NO: 48 - protein, 10 aa, synthetic construct
AGYKGNGDAA

SEQ ID NO: 49 - protein, 119 aa, synthetic construct
QVQLVQSGAEVKKPGASVKVSCKASGYTFTTYNMHWVRQAPGQGLEWMGAIFPGNGHISYAQKFQGRVTMTVDTSTSTVYMELSSLRSEDTAVYYCARGDYDESYFDYWGQGTLVTVSS

SEQ ID NO: 50 - protein, 107 aa, synthetic construct
DIQMTQSPSSLSASVGDRVTITCKASEGIYNRLAWYQQKPGKAPKLLLYGVISLETGVPSRFSGSGSGTDYTLTISSLQPEDFATYYCQQYWSIPWTFGGGTKVEIK

SEQ ID NO: 51 - protein, 107 aa, synthetic construct
DIQMTQSPSSLSASVGDRVTITCKASEGIYNRLAWYQQKPGKAPKLLLYGVISLETGVPSRFSGSGSGTDYTLTISSLQCEDVATYYCQQYWSIPWTFGGGTKVEIK

SEQ ID NO: 52 - protein, 118 aa, synthetic construct
QVQLQQSGAELVKPGASVKISCKASGYAFRSYWVNWVKQRPGKGLEWIGQIFPGDGDINYSGSFKGKATLTADKSSSTAYMQLSSLTSEDSAVYFCARMDFYSWFPYWGQGTLVTVSA

SEQ ID NO: 53 - protein, 113 aa, synthetic construct
DIVMTQSPDSLAVSLGERATINCKSSQSLLNSGNQKNDLAWYQQKPGQPPKLLIYGASTRESGVPDRFSGSGSGTDFTLTISSLQAEDVAVYYCQNDHSYPLTFGQGTKLEIK

SEQ ID NO: 54 - protein, 113 aa, synthetic construct
DIVMTQSPDSLAVSLGERATINCKSSQSLLNSGNQKNDLAWYQQKPGQPPKLLIYGASTRESGVPDRFSGSGSGTDFTLTISSLQCEDVAVYYCQNDHSYPLTFGQGTKLEIK

SEQ ID NO: 55 - protein, 115 aa, synthetic construct
QELKESGGGLVTPGGTLTLTCTASGFSLSNYYINWVRQAPGKGLEYIGFIPMYGTTHYANWAKGRFTISRTSTTVDLKMASLTASDTATYFCARGIASMFYPSIWGPGTLVTVSS

SEQ ID NO: 56 - protein, 108 aa, synthetic construct
DVVMTQSPSSLSASVGDRVTITCQASEDIHGYLAWYQQKPGKAPKLLIYYASTLASGVPSRFSGSGSGTDFTLTISSLQPEDFATYYCLGDGSTSGITFGQGTKVEIK

SEQ ID NO: 57 - protein, 108 aa, synthetic construct
DVVMTQSPSSLSASVGDRVTITCQASEDIHGYLAWYQQKPGKAPKLLIYYASTLASGVPSRFSGSGSGTDFTLTISSLQCEDVATYYCLGDGSTSGITFGQGTKVEIK

SEQ ID NO: 58 - protein, 119 aa, synthetic construct
EVQLVESGGGLIQPGGSLRLSCAASGIDLSSSTMAWVRQAPGKGLEWIGIIASSGSAYYAGWAKGRFTISRDNSKNTLYLQMNSLRAEDTAVYYCVRQVPGYNDNRYVWGQGTLVTVSS

SEQ ID NO: 59 - protein, 110 aa, synthetic construct
AIVMTQSPSSLSASVGDRVTITCQASESVYNANSCSWYQQKPGKAPKLLIYDASDLASGVPSRFSGSGSGTDFTLTISSLQPEDFATYYCAGYKGNGDAAFGQGTKVEIK

SEQ ID NO: 60 - protein, 110 aa, synthetic construct
AIVMTQSPSSLSASVGDRVTITCQASESVYNANSCSWYQQKPGKAPKLLIYDASDLASGVPSRFSGSGSGTDFTLTISSLQCEDVATYYCAGYKGNGDAAFGQGTKVEIK

SEQ ID NO: 61 - protein, 449 aa, synthetic construct
QVQLVQSGAEVKKPGASVKVSCKASGYTFTTYNMHWVRQAPGQGLEWMGAIFPGNGHISYAQKFQGRVTMTVDTSTSTVYMELSSLRSEDTAVYYCARGDYDESYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 449 aa, synthetic construct
QVQLVQSGAEVKKPGASVKVSCKASGYTFTTYNMHWVRQAPGQGLEWMGAIFPGNGHISYAQKFQGRVTMTVDTSTSTVYMELSSLRSEDTAVYYCARGDYDESYFDYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 449 aa, synthetic construct
QVQLVQSGAEVKKPGASVKVSCKASGYTFTTYNMHWVRQAPGQGLEWMGAIFPGNGHISYAQKFQGRVTMTVDTSTSTVYMELSSLRSEDTAVYYCARGDYDESYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGSSVFLFPPKPKDTLMISRTPEVTCVVVDVSQEDPEVQFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 449 aa, synthetic construct
QVQLVQSGAEVKKPGASVKVSCKASGYTFTTYNMHWVRQAPGQGLEWMGAIFPGNGHISYAQKFQGRVTMTVDTSTSTVYMELSSLRSEDTAVYYCARGDYDESYFDYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGSSVFLFPPKPKDTLMISRTPEVTCVVVDVSQEDPEVQFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 214 aa, synthetic construct
DIQMTQSPSSLSASVGDRVTITCKASEGIYNRLAWYQQKPGKAPKLLLYGVISLETGVPSRFSGSGSGTDYTLTISSLQPEDFATYYCQQYWSIPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 214 aa, synthetic construct
DIQMTQSPSSLSASVGDRVTITCKASEGIYNRLAWYQQKPGKAPKLLLYGVISLETGVPSRFSGSGSGTDYTLTISSLQCEDVATYYCQQYWSIPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 448 aa, synthetic construct
QVQLVQSGAEVKKPGSSVKVSCKASGYAFRSYWISWVRQAPGQGLEWMGQIFPGDGDINYAQKFQGRVTITADKSTSTAYMELSSLRSEDTAVYYCARMDFYSWFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 448 aa, synthetic construct
QVQLVQSGAEVKKPGSSVKVSCKASGYAFRSYWISWVRQAPGQGLEWMGQIFPGDGDINYAQKFQGRVTITADKSTSTAYMELSSLRSEDTAVYYCARMDFYSWFPYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 448 aa, synthetic construct
QVQLVQSGAEVKKPGSSVKVSCKASGYAFRSYWISWVRQAPGQGLEWMGQIFPGDGDINYAQKFQGRVTITADKSTSTAYMELSSLRSEDTAVYYCARMDFYSWFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGSSVFLFPPKPKDTLMISRTPEVTCVVVDVSQEDPEVQFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 449 aa, synthetic construct; identical to SEQ ID NO: 64

SEQ ID NO: 71 - protein, 220 aa, synthetic construct
DIVMTQSPDSLAVSLGERATINCKSSQSLLNSGNQKNDLAWYQQKPGQPPKLLIYGASTRESGVPDRFSGSGSGTDFTLTISSLQAEDVAVYYCQNDHSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 220 aa, synthetic construct
DIVMTQSPDSLAVSLGERATINCKSSQSLLNSGNQKNDLAWYQQKPGQPPKLLIYGASTRESGVPDRFSGSGSGTDFTLTISSLQCEDVAVYYCQNDHSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 448 aa, synthetic construct
EVQLVESGGGLIQPGGSLRLSCAASGFSLSNYYINWVRQAPGKGLEWIGFIPMYGTTHYANWAKGRFTISRDNSKNTLYLQMNSLRAEDTAVYYCARGIASMFYPSIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 448 aa, synthetic construct
EVQLVESGGGLIQPGGSLRLSCAASGFSLSNYYINWVRQAPGKGLEWIGFIPMYGTTHYANWAKGRFTISRDNSKNTLYLQMNSLRAEDTAVYYCARGIASMFYPSIWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 448 aa, synthetic construct
EVQLVESGGGLIQPGGSLRLSCAASGFSLSNYYINWVRQAPGKGLEWIGFIPMYGTTHYANWAKGRFTISRDNSKNTLYLQMNSLRAEDTAVYYCARGIASMFYPSIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGSSVFLFPPKPKDTLMISRTPEVTCVVVDVSQEDPEVQFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 448 aa, synthetic construct
EVQLVESGGGLIQPGGSLRLSCAASGFSLSNYYINWVRQAPGKGLEWIGFIPMYGTTHYANWAKGRFTISRDNSKNTLYLQMNSLRAEDTAVYYCARGIASMFYPSIWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGSSVFLFPPKPKDTLMISRTPEVTCVVVDVSQEDPEVQFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 215 aa, synthetic construct
DVVMTQSPSSLSASVGDRVTITCQASEDIHGYLAWYQQKPGKAPKLLIYYASTLASGVPSRFSGSGSGTDFTLTISSLQPEDFATYYCLGDGSTSGITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 215 aa, synthetic construct
DVVMTQSPSSLSASVGDRVTITCQASEDIHGYLAWYQQKPGKAPKLLIYYASTLASGVPSRFSGSGSGTDFTLTISSLQCEDVATYYCLGDGSTSGITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 449 aa, synthetic construct
EVQLVESGGGLIQPGGSLRLSCAASGIDLSSSTMAWVRQAPGKGLEWIGIIASSGSAYYAGWAKGRFTISRDNSKNTLYLQMNSLRAEDTAVYYCVRQVPGYNDNRYVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 449 aa, synthetic construct
EVQLVESGGGLIQPGGSLRLSCAASGIDLSSSTMAWVRQAPGKGLEWIGIIASSGSAYYAGWAKGRFTISRDNSKNTLYLQMNSLRAEDTAVYYCVRQVPGYNDNRYVWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 449 aa, synthetic construct
EVQLVESGGGLIQPGGSLRLSCAASGIDLSSSTMAWVRQAPGKGLEWIGIIASSGSAYYAGWAKGRFTISRDNSKNTLYLQMNSLRAEDTAVYYCVRQVPGYNDNRYVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGSSVFLFPPKPKDTLMISRTPEVTCVVVDVSQEDPEVQFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 449 aa, synthetic construct
EVQLVESGGGLIQPGGSLRLSCAASGIDLSSSTMAWVRQAPGKGLEWIGIIASSGSAYYAGWAKGRFTISRDNSKNTLYLQMNSLRAEDTAVYYCVRQVPGYNDNRYVWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGSSVFLFPPKPKDTLMISRTPEVTCVVVDVSQEDPEVQFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 217 aa, synthetic construct
AIVMTQSPSSLSASVGDRVTITCQASESVYNANSCSWYQQKPGKAPKLLIYDASDLASGVPSRFSGSGSGTDFTLTISSLQPEDFATYYCAGYKGNGDAAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - protein, 217 aa, synthetic construct
AIVMTQSPSSLSASVGDRVTITCQASESVYNANSCSWYQQKPGKAPKLLIYDASDLASGVPSRFSGSGSGTDFTLTISSLQCEDVATYYCAGYKGNGDAAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 118 aa, synthetic construct
EVQLVESGGGLIQPGGSLRLSCAASGFTLSNYYINWVRQAPGKGLEWIGFIPMYGTTHYANWAKGRFTISRDNSKNTLYLQMNSLRAEDTAVYYCARGIASMFYPSIWGQGTLVTVSS

SEQ ID NO: 86 - protein, 214 aa, synthetic construct
DIQMTQSSSSFSVSLGDRVTLSCKASEGIYNRLAWYQQKPGNAPRLLISGVISLETGVPSRFSGSGSGKDYTLSITSLQTEDVATYYCQQYWSIPWTFGGGNKLEINRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - protein, 448 aa, synthetic construct
QVQLQQSGAELVKPGASVKISCKASGYAFRSYWVNWVKQRPGKGLEWIGQIFPGDGDINYSGSFKGKATLTADKSSSTAYMQLSSLTSEDSAVYFCARMDFYSWFPYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 220 aa, synthetic construct
DIVMTQSPSSLSVSAGEKVTMSCKSSQSLLNSGNQKNDLAWFQQKPGQPPKLLIYGASTRESGVPDRFTGSGSGTDFTLTISSVQAEDLAVYYCQNDHSYPLTFGTGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 89 - protein, 445 aa, synthetic construct
QELKESGGGLVTPGGTLTLTCTASGFSLSNYYINWVRQAPGKGLEYIGFIPMYGTTHYANWAKGRFTISRTSTTVDLKMASLTASDTATYFCARGIASMFYPSIWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 215 aa, synthetic construct
DVVMTQTPSSTSAAVGGAVTIKCQASEDIHGYLAWYQQKPGQPPKLLIYYASTLASGVSSRFKGSGSGTEYTLTISGVQREDAATYYCLGDGSTSGITFGGGTEFEILRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 447 aa, synthetic construct
QSVEESGGRLVTPGTPLTLTCTVSGIDLSSSTMAWVRQAPGKGLEYIGIIASSGSAYYAGWAKGRFTISKTSSTTVDLKITSPTTEDTATYFCVRQVPGYNDNRYVWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 217 aa, synthetic construct
AIVMTQTPSSVSAAVGGTVTIHCQASESVYNANSCSWFQQKPGQRPKLLIYDASDLASGVPSRFRGSGSGTQFTLTINNVQREDVATYFCAGYKGNGDAAFGGGTELEILRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 118 aa, synthetic construct
EVQLVESGGGLIQPGGSLRLSCAASGFTLSNYYINWVRQAPGKGLEWIGFIPMYGTTHYANSVKGRFTISRDNSKNTLYLQMNSLRAEDTAVYYCARGIASMFYPSIWGQGTLVTVSS

SEQ ID NO: 94 - protein, 118 aa, synthetic construct
EVQLVESGGGLIQPGGSLRLSCAASGFTLSNYYINWVRQAPGKGLEWIGFIPMYGTTHYANWVKGRFTISRDNSKNTLYLQMNSLRAEDTAVYYCARGIASMFYPSIWGQGTLVTVSS

SEQ ID NO: 95 - protein, 118 aa, synthetic construct
EVQLVESGGGLIQPGGSLRLSCAASGFTVSNYYINWVRQAPGKGLEWIGFIPMYGTTHYADSVKGRFTISRDNSKNTLYLQMNSLRAEDTAVYYCARGIASMFYPSIWGQGTLVTVSS

SEQ ID NO: 96 - protein, 118 aa, synthetic construct
EVQLVESGGGLIQPGGSLRLSCAASGFTVSNYYINWVRQAPGKGLEWIGFIPMYGTTHYANSVKGRFTISRDNSKNTLYLQMNSLRAEDTAVYYCARGIASMFYPSIWGQGTLVTVSS

SEQ ID NO: 97 - protein, 118 aa, synthetic construct
EVQLVESGGGLIQPGGSLRLSCAASGFTVSNYYINWVRQAPGKGLEWIGFIPMYGTTHYANWVKGRFTISRDNSKNTLYLQMNSLRAEDTAVYYCARGIASMFYPSIWGQGTLVTVSS

SEQ ID NO: 98 - protein, 118 aa, synthetic construct
EVQLVESGGGLIQPGGSLRLSCAASGFTLSNYYINWVRQAPGKGLEWVGFIPMYGTTHYADSVKGRFTISRDNSKNTLYLQMNSLRAEDTAVYYCARGIASMFYPSIWGQGTLVTVSS

SEQ ID NO: 99 - protein, 118 aa, synthetic construct
EVQLVESGGGLIQPGGSLRLSCAASGFTLSNYYINWVRQAPGKGLEWVGFIPMYGTTHYANSVKGRFTISRDNSKNTLYLQMNSLRAEDTAVYYCARGIASMFYPSIWGQGTLVTVSS

SEQ ID NO: 100 - protein, 118 aa, synthetic construct
EVQLVESGGGLIQPGGSLRLSCAASGFTLSNYYINWVRQAPGKGLEWVGFIPMYGTTHYANWVKGRFTISRDNSKNTLYLQMNSLRAEDTAVYYCARGIASMFYPSIWGQGTLVTVSS

SEQ ID NO: 101 - protein, 118 aa, synthetic construct
EVQLVESGGGLIQPGGSLRLSCAASGFTVSNYYINWVRQAPGKGLEWVGFIPMYGTTHYADSVKGRFTISRDNSKNTLYLQMNSLRAEDTAVYYCARGIASMFYPSIWGQGTLVTVSS

SEQ ID NO: 102 - protein, 118 aa, synthetic construct
EVQLVESGGGLIQPGGSLRLSCAASGFTVSNYYINWVRQAPGKGLEWVGFIPMYGTTHYANSVKGRFTISRDNSKNTLYLQMNSLRAEDTAVYYCARGIASMFYPSIWGQGTLVTVSS

SEQ ID NO: 103 - protein, 118 aa, synthetic construct
EVQLVESGGGLIQPGGSLRLSCAASGFTVSNYYINWVRQAPGKGLEWVGFIPMYGTTHYANWVKGRFTISRDNSKNTLYLQMNSLRAEDTAVYYCARGIASMFYPSIWGQGTLVTVSS

SEQ ID NO: 104 - protein, 108 aa, synthetic construct
DVVMTQSPSSLSASVGDRVTITCQASEDIHGYLNWYQQKPGKAPKLLIYYASTLASGVPSRFSGSGSGTDFTLTISSLQPEDFATYYCLGDGSTSGITFGQGTKVEIK

SEQ ID NO: 105 - protein, 108 aa, synthetic construct
DVVMTQSPSSLSASVGDRVTITCQASEDIHGYLAWYQQKPGKAPKLLIYYASTLQSGVPSRFSGSGSGTDFTLTISSLQPEDFATYYCLGDGSTSGITFGQGTKVEIK

SEQ ID NO: 106 - protein, 108 aa, synthetic construct
DVVMTQSPSSLSASVGDRVTITCQASEDIHGYLNWYQQKPGKAPKLLIYYASTLQSGVPSRFSGSGSGTDFTLTISSLQPEDFATYYCLGDGSTSGITFGQGTKVEIK

SEQ ID NO: 107 - protein, 108 aa, synthetic construct
DVVMTQSPSSLSASVGDRVTITCRASEDIHGYLAWYQQKPGKAPKLLIYYASTLASGVPSRFSGSGSGTDFTLTISSLQPEDFATYYCLGDGSTSGITFGQGTKVEIK

SEQ ID NO: 108 - protein, 108 aa, synthetic construct
DVVMTQSPSSLSASVGDRVTITCRASEDIHGYLNWYQQKPGKAPKLLIYYASTLASGVPSRFSGSGSGTDFTLTISSLQPEDFATYYCLGDGSTSGITFGQGTKVEIK

SEQ ID NO: 109 - protein, 108 aa, synthetic construct
DVVMTQSPSSLSASVGDRVTITCRASEDIHGYLAWYQQKPGKAPKLLIYYASTLQSGVPSRFSGSGSGTDFTLTISSLQPEDFATYYCLGDGSTSGITFGQGTKVEIK

SEQ ID NO: 110 - protein, 108 aa, synthetic construct
DVVMTQSPSSLSASVGDRVTITCRASEDIHGYLNWYQQKPGKAPKLLIYYASTLQSGVPSRFSGSGSGTDFTLTISSLQPEDFATYYCLGDGSTSGITFGQGTKVEIK

SEQ ID NO: 111 - protein, 448 aa, synthetic construct
QVQLVQSGAEVKKPGASVKVSCKASGYTFTTYNMHWVRQAPGQGLEWMGAIFPGNGHISYAQKFQGRVTMTVDTSTSTVYMELSSLRSEDTAVYYCARGDYDESYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 112 - protein, 448 aa, synthetic construct
QVQLVQSGAEVKKPGASVKVSCKASGYTFTTYNMHWVRQAPGQGLEWMGAIFPGNGHISYAQKFQGRVTMTVDTSTSTVYMELSSLRSEDTAVYYCARGDYDESYFDYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 113 - protein, 448 aa, synthetic construct
QVQLVQSGAEVKKPGASVKVSCKASGYTFTTYNMHWVRQAPGQGLEWMGAIFPGNGHISYAQKFQGRVTMTVDTSTSTVYMELSSLRSEDTAVYYCARGDYDESYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGSSVFLFPPKPKDTLMISRTPEVTCVVVDVSQEDPEVQFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 114 - protein, 448 aa, synthetic construct
QVQLVQSGAEVKKPGASVKVSCKASGYTFTTYNMHWVRQAPGQGLEWMGAIFPGNGHISYAQKFQGRVTMTVDTSTSTVYMELSSLRSEDTAVYYCARGDYDESYFDYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGSSVFLFPPKPKDTLMISRTPEVTCVVVDVSQEDPEVQFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 115 - protein, 447 aa, synthetic construct
QVQLVQSGAEVKKPGSSVKVSCKASGYAFRSYWISWVRQAPGQGLEWMGQIFPGDGDINYAQKFQGRVTITADKSTSTAYMELSSLRSEDTAVYYCARMDFYSWFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 116 - protein, 447 aa, synthetic construct
QVQLVQSGAEVKKPGSSVKVSCKASGYAFRSYWISWVRQAPGQGLEWMGQIFPGDGDINYAQKFQGRVTITADKSTSTAYMELSSLRSEDTAVYYCARMDFYSWFPYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 117 - protein, 447 aa, synthetic construct
QVQLVQSGAEVKKPGSSVKVSCKASGYAFRSYWISWVRQAPGQGLEWMGQIFPGDGDINYAQKFQGRVTITADKSTSTAYMELSSLRSEDTAVYYCARMDFYSWFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGSSVFLFPPKPKDTLMISRTPEVTCVVVDVSQEDPEVQFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 118 - protein, 448 aa, synthetic construct; identical to SEQ ID NO: 114

SEQ ID NO: 119 - protein, 447 aa, synthetic construct
EVQLVESGGGLIQPGGSLRLSCAASGFSLSNYYINWVRQAPGKGLEWIGFIPMYGTTHYANWAKGRFTISRDNSKNTLYLQMNSLRAEDTAVYYCARGIASMFYPSIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 120 - protein, 448 aa, synthetic construct; identical to SEQ ID NO: 74

SEQ ID NO: 121 - protein, 447 aa, synthetic construct
EVQLVESGGGLIQPGGSLRLSCAASGFSLSNYYINWVRQAPGKGLEWIGFIPMYGTTHYANWAKGRFTISRDNSKNTLYLQMNSLRAEDTAVYYCARGIASMFYPSIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGSSVFLFPPKPKDTLMISRTPEVTCVVVDVSQEDPEVQFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 122 - protein, 447 aa, synthetic construct
EVQLVESGGGLIQPGGSLRLSCAASGFSLSNYYINWVRQAPGKGLEWIGFIPMYGTTHYANWAKGRFTISRDNSKNTLYLQMNSLRAEDTAVYYCARGIASMFYPSIWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGSSVFLFPPKPKDTLMISRTPEVTCVVVDVSQEDPEVQFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 123 - protein, 448 aa, synthetic construct
EVQLVESGGGLIQPGGSLRLSCAASGIDLSSSTMAWVRQAPGKGLEWIGIIASSGSAYYAGWAKGRFTISRDNSKNTLYLQMNSLRAEDTAVYYCVRQVPGYNDNRYVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 124 - protein, 448 aa, synthetic construct
EVQLVESGGGLIQPGGSLRLSCAASGIDLSSSTMAWVRQAPGKGLEWIGIIASSGSAYYAGWAKGRFTISRDNSKNTLYLQMNSLRAEDTAVYYCVRQVPGYNDNRYVWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 125 - protein, 448 aa, synthetic construct
EVQLVESGGGLIQPGGSLRLSCAASGIDLSSSTMAWVRQAPGKGLEWIGIIASSGSAYYAGWAKGRFTISRDNSKNTLYLQMNSLRAEDTAVYYCVRQVPGYNDNRYVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGSSVFLFPPKPKDTLMISRTPEVTCVVVDVSQEDPEVQFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 126 - protein, 448 aa, synthetic construct
EVQLVESGGGLIQPGGSLRLSCAASGIDLSSSTMAWVRQAPGKGLEWIGIIASSGSAYYAGWAKGRFTISRDNSKNTLYLQMNSLRAEDTAVYYCVRQVPGYNDNRYVWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGSSVFLFPPKPKDTLMISRTPEVTCVVVDVSQEDPEVQFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 127 - DNA, 15 bp, synthetic construct
  source 1..15: mol_type = other DNA
acgtacaacatgcat

SEQ ID NO: 128 - DNA, 51 bp, synthetic construct
  source 1..51: mol_type = other DNA
gccatttttcccggaaacggccacatctcgtacgcccaaaagttccagggc

SEQ ID NO: 129 - DNA, 30 bp, synthetic construct
  source 1..30: mol_type = other DNA
ggggattacgacgaaagctacttcgactac

SEQ ID NO: 130 - DNA, 33 bp, synthetic construct
  source 1..33: mol_type = other DNA
aaagcctccgaggggatctacaacagactcgcc

SEQ ID NO: 131 - DNA, 21 bp, synthetic construct
  source 1..21: mol_type = other DNA
ggagtgatctccctggaaact

SEQ ID NO: 132 - DNA, 21 bp, synthetic construct
  source 1..21: mol_type = other DNA
ggcgtgatctctctggaaaca

SEQ ID NO: 133 - DNA, 27 bp, synthetic construct
  source 1..27: mol_type = other DNA
cagcagtactggtcgattccgtggact

SEQ ID NO: 134 - DNA, 27 bp, synthetic construct
  source 1..27: mol_type = other DNA
cagcagtactggtctatcccctggacc

SEQ ID NO: 135 - DNA, 24 bp, synthetic construct
  source 1..24: mol_type = other DNA
ggatacaccttcaccacgtacaac

SEQ ID NO: 136 - DNA, 24 bp, synthetic construct
  source 1..24: mol_type = other DNA
atttttcccggaaacggccacatc

SEQ ID NO: 137 - DNA, 36 bp, synthetic construct
  source 1..36: mol_type = other DNA
gcgcggggggattacgacgaaagctacttcgactac

SEQ ID NO: 138 - DNA, 18 bp, synthetic construct
  source 1..18: mol_type = other DNA
gaggggatctacaacaga

SEQ ID NO: 139 - skipped

SEQ ID NO: 140 - DNA, 27 bp, synthetic construct
  source 1..27: mol_type = other DNA
cagcagtactggtcgattccgtggact

SEQ ID NO: 141 - DNA, 27 bp, synthetic construct
  source 1..27: mol_type = other DNA
cagcagtactggtctatcccctggacc

SEQ ID NO: 142 - DNA, 15 bp, synthetic construct
  source 1..15: mol_type = other DNA
tcgtactggatctcc

SEQ ID NO: 143 - DNA, 51 bp, synthetic construct
  source 1..51: mol_type = other DNA
cagatctttcctggggacggcgacattaactacgcgcagaagttccagggc

SEQ ID NO: 144 - DNA, 27 bp, synthetic construct
  source 1..27: mol_type = other DNA
atggacttctactcctggttcccgtac

SEQ ID NO: 145 - DNA, 51 bp, synthetic construct
  source 1..51: mol_type = other DNA
aaatccagccagtcgctgctgaactcggggaaccagaagaatgatctggcc

SEQ ID NO: 146 - DNA, 21 bp, synthetic construct
  source 1..21: mol_type = other DNA
ggagcatcaactcgcgaatcc

SEQ ID NO: 147 - DNA, 27 bp, synthetic construct
  source 1..27: mol_type = other DNA
cagaacgaccactcctatccgctgact

SEQ ID NO: 148 - DNA, 24 bp, synthetic construct
  source 1..24: mol_type = other DNA
ggatacgcattccggtcgtactgg

SEQ ID NO: 149 - DNA, 24 bp, synthetic construct
  source 1..24: mol_type = other DNA
atctttcctggggacggcgacatt

SEQ ID NO: 150 - DNA, 33 bp, synthetic construct
  source 1..33: mol_type = other DNA
gctcggatggacttctactcctggttcccgtac

SEQ ID NO: 151 - DNA, 27 bp, synthetic construct
  source 1..27: mol_type = other DNA
cagaacgaccactcctatccgctgact

SEQ ID NO: 152 - DNA, 18 bp, synthetic construct
  source 1..18: mol_type = other DNA
gaggacatccacggctat

SEQ ID NO: 153 - skipped

SEQ ID NO: 154 - DNA, 15 bp, synthetic construct
  source 1..15: mol_type = other DNA
aactactacatcaac

SEQ ID NO: 155 - DNA, 48 bp, synthetic construct
  source 1..48: mol_type = other DNA
ttcatccctatgtacggcaccacacactacgccaactgggccaagggc

SEQ ID NO: 156 - DNA, 30 bp, synthetic construct
  source 1..30: mol_type = other DNA
ggaatcgccagcatgttctacccttctatc

SEQ ID NO: 157 - DNA, 33 bp, synthetic construct
  source 1..33: mol_type = other DNA
caggccagcgaggacatccacggctatctggcc

SEQ ID NO: 158 - DNA, 21 bp, synthetic construct
  source 1..21: mol_type = other DNA
tacgcctctacactggccagc

SEQ ID NO: 159 - DNA, 30 bp, synthetic construct
  source 1..30: mol_type = other DNA
ctcggagatggcagcaccagcggcatcaca

SEQ ID NO: 160 - DNA, 24 bp, synthetic construct
  source 1..24: mol_type = other DNA
ggcttcagcctgagcaactactac

SEQ ID NO: 161 - DNA, 21 bp, synthetic construct
  source 1..21: mol_type = other DNA
atccctatgtacggcaccaca

SEQ ID NO: 162 - DNA, 36 bp, synthetic construct
  source 1..36: mol_type = other DNA
gccagaggaatcgccagcatgttctacccttctatc

SEQ ID NO: 163 - DNA, 30 bp, synthetic construct; identical to SEQ ID NO: 159
  source 1..30: mol_type = other DNA

SEQ ID NO: 164 - DNA, 24 bp, synthetic construct
  source 1..24: mol_type = other DNA
gagagcgtgtacaacgccaacagc

SEQ ID NO: 165 - skipped

SEQ ID NO: 166 - DNA, 15 bp, synthetic construct
  source 1..15: mol_type = other DNA
agctctacaatggcc

SEQ ID NO: 167 - DNA, 48 bp, synthetic construct
  source 1..48: mol_type = other DNA
atcattgccagcagcggctctgcctattatgccggatgggccaagggc

SEQ ID NO: 168 - DNA, 33 bp, synthetic construct
  source 1..33: mol_type = other DNA
caggtgcccggctacaacgacaacagatatgtg

SEQ ID NO: 169 - DNA, 39 bp, synthetic construct
  source 1..39: mol_type = other DNA
caggccagcgagagcgtgtacaacgccaacagctgtagc

SEQ ID NO: 170 - DNA, 21 bp, synthetic construct
  source 1..21: mol_type = other DNA
gatgcctctgatctggccagc

SEQ ID NO: 171 - DNA, 30 bp, synthetic construct
  source 1..30: mol_type = other DNA
gccggctacaaaggcaatggcgacgccgct

SEQ ID NO: 172 - DNA, 24 bp, synthetic construct
  source 1..24: mol_type = other DNA
ggcatcgatctgagcagctctaca

SEQ ID NO: 173 - DNA, 21 bp, synthetic construct
  source 1..21: mol_type = other DNA
attgccagcagcggctctgcc

SEQ ID NO: 174 - DNA, 39 bp, synthetic construct
  source 1..39: mol_type = other DNA
gttagacaggtgcccggctacaacgacaacagatatgtg

SEQ ID NO: 175 - DNA, 30 bp, synthetic construct; identical to SEQ ID NO: 171
  source 1..30: mol_type = other DNA

SEQ ID NO: 176-177 - skipped (2 entries)

SEQ ID NO: 178 - DNA, 357 bp, synthetic construct
  source 1..357: mol_type = other DNA
caagtgcagcttgtgcagtccggagctgaagtcaagaagcctggtgccagcgtgaaagtgtcctgcaaggcctccggatacaccttcaccacgtacaacatgcattgggtcagacaggcaccgggacagggtctggaatggatgggggccatttttcccggaaacggccacatctcgtacgcccaaaagttccagggccgcgtgaccatgactgtggacacctccacctccaccgtgtacatggagctctcaagcctgaggtccgaggatactgccgtgtactattgtgcgcggggggattacgacgaaagctacttcgactactggggccagggcactctggtcaccgtgtcgtca

SEQ ID NO: 179 - DNA, 321 bp, synthetic construct
  source 1..321: mol_type = other DNA
gacattcagatgacccaatccccctcatcactgtcggcttccgtgggcgaccgcgtgaccatcacctgtaaagcctccgaggggatctacaacagactcgcctggtatcagcagaagccaggaaaggcccctaagctccttctgtacggagtgatctccctggaaactggagtgccgagccggtttagcggcagcggttccggaactgactacacgttgaccatctcctcgctgcaacccgaagatttcgcgacctactactgccagcagtactggtcgattccgtggactttcggtggcgggaccaaggtcgagatcaaa

SEQ ID NO: 180 - DNA, 321 bp, synthetic construct
  source 1..321: mol_type = other DNA
gatattcagatgacacagagccctagcagcctgagcgcctctgtgggcgatagagtgacaatcacatgcaaggccagcgagggcatctacaacagactggcctggtatcagcagaagcccggaaaggctcctaagctgctgctgtacggcgtgatctctctggaaacaggcgtgccaagcagattttctggcagcggctctggcaccgactacaccctgacaatttctagcctgcagtgcgaggacgtggccacctactactgccagcagtactggtctatcccctggacctttggcggaggcaccaaggtggaaatcaag

SEQ ID NO: 181 - DNA, 354 bp, synthetic construct
  source 1..354: mol_type = other DNA
caagtgcagctggtgcagtctggtgccgaggtcaagaagccaggctccagcgtgaaagtgtcctgcaaggcctcgggatacgcattccggtcgtactggatctcctgggtccgccaggcccccggacaaggccttgaatggatggggcagatctttcctggggacggcgacattaactacgcgcagaagttccagggcagagtgaccatcactgccgataagagcacctccactgcgtacatggaactgagctccctgaggtccgaggataccgccgtgtactattgcgctcggatggacttctactcctggttcccgtactggggacagggtaccctcgtcaccgtgtcatca

SEQ ID NO: 182 - DNA, 339 bp, synthetic construct
  source 1..339: mol_type = other DNA
gacattgtgatgacccagtcccccgattcccttgccgtgtccctcggtgaacgggccactatcaactgcaaatccagccagtcgctgctgaactcggggaaccagaagaatgatctggcctggtaccagcagaagcctggccagcccccaaagctgctcatctacggagcatcaactcgcgaatccggagtgccggacagattttccgggagcggatcaggcaccgacttcaccttgaccatttcgagcctgcaagcggaggacgtcgctgtgtactactgtcagaacgaccactcctatccgctgactttcggacaaggcaccaagctggagatcaaa

SEQ ID NO: 183 - DNA, 339 bp, synthetic construct
  source 1..339: mol_type = other DNA
gacattgtgatgacccagtcccccgattcccttgccgtgtccctcggtgaacgggccactatcaactgcaaatccagccagtcgctgctgaactcggggaaccagaagaatgatctggcctggtaccagcagaagcctggccagcccccaaagctgctcatctacggagcatcaactcgcgaatccggagtgccggacagattttccgggagcggatcaggcaccgacttcaccttgaccatttcgagcctgcaatgcgaggacgtcgctgtgtactactgtcagaacgaccactcctatccgctgactttcggacaaggcaccaagctggagatcaaa

SEQ ID NO: 184 - DNA, 354 bp, synthetic construct
  source 1..354: mol_type = other DNA
gaagtgcagctggttgaatctggcggaggactgattcagcctggcggctctctgagactgtcttgtgccgcctctggcttcagcctgagcaactactacatcaactgggtccgacaggcccctggcaaaggactggaatggatcggcttcatccctatgtacggcaccacacactacgccaactgggccaagggcagattcaccatcagccgggacaacagcaagaacaccctgtacctgcagatgaacagcctgagagccgaggacaccgccgtgtactattgtgccagaggaatcgccagcatgttctacccttctatctggggccagggcaccctggtcacagttagttct

SEQ ID NO: 185 - DNA, 324 bp, synthetic construct
  source 1..324: mol_type = other DNA
gacgttgtgatgacacagagccctagcagcctgagcgcctctgtgggcgatagagtgacaatcacctgtcaggccagcgaggacatccacggctatctggcctggtatcagcagaagcctggaaaggcccctaagctgctgatctactacgcctctacactggccagcggcgtgccaagcagattttctggctctggcagcggcaccgacttcaccctgacaatttctagcctccagcctgaggacttcgccacctactattgtctcggagatggcagcaccagcggcatcacatttggccagggcaccaaggtggaaatcaaa

SEQ ID NO: 186 - DNA, 324 bp, synthetic construct
  source 1..324: mol_type = other DNA
gacgttgtgatgacacagagccctagcagcctgagcgcctctgtgggcgatagagtgacaatcacctgtcaggccagcgaggacatccacggctatctggcctggtatcagcagaagcctggaaaggcccctaagctgctgatctactacgcctctacactggccagcggcgtgccaagcagattttctggctctggcagcggcaccgacttcaccctgacaatttctagcctccagtgcgaggacgtggccacctactattgtctcggagatggcagcaccagcggcatcacatttggccagggcaccaaggtggaaatcaaa

SEQ ID NO: 187 - DNA, 357 bp, synthetic construct
  source 1..357: mol_type = other DNA
gaagtgcagctggttgaatctggcggaggactgattcagcctggcggctctctgagactgtcttgtgccgcctctggcatcgatctgagcagctctacaatggcctgggttcgacaggcccctggcaaaggactggaatggatcggcatcattgccagcagcggctctgcctattatgccggatgggccaagggcagattcaccatcagccgggacaacagcaagaacaccctgtacctgcagatgaacagcctgagagccgaggacaccgccgtgtactattgtgttagacaggtgcccggctacaacgacaacagatatgtgtggggccagggcaccctggtcacagttagttct

SEQ ID NO: 188 - DNA, 330 bp, synthetic construct
  source 1..330: mol_type = other DNA
gctattgtgatgacacagagccctagcagcctgagcgcctctgtgggcgatagagtgacaatcacctgtcaggccagcgagagcgtgtacaacgccaacagctgtagctggtatcagcagaagcctggcaaggcccctaagctgctgatctacgatgcctctgatctggccagcggcgtgccaagcagattttctggctctggcagcggcaccgacttcaccctgacaatttctagcctccagcctgaggacttcgccacctactattgtgccggctacaaaggcaatggcgacgccgcttttggccagggcacaaaggtggaaatcaaa

SEQ ID NO: 189 - DNA, 330 bp, synthetic construct
  source 1..330: mol_type = other DNA
gctattgtgatgacacagagccctagcagcctgagcgcctctgtgggcgatagagtgacaatcacctgtcaggccagcgagagcgtgtacaacgccaacagctgtagctggtatcagcagaagcctggcaaggcccctaagctgctgatctacgatgcctctgatctggccagcggcgtgccaagcagattttctggctctggcagcggcaccgacttcaccctgacaatttctagcctccagtgcgaggacgtggccacctactattgtgccggctacaaaggcaatggcgacgccgcttttggccagggcacaaaggtggaaatcaaa

SEQ ID NO: 190 - DNA, 1347 bp, synthetic construct
  source 1..1347: mol_type = other DNA
caagtgcagcttgtgcagtccggagctgaagtcaagaagcctggtgccagcgtgaaagtgtcctgcaaggcctccggatacaccttcaccacgtacaacatgcattgggtcagacaggcaccgggacagggtctggaatggatgggggccatttttcccggaaacggccacatctcgtacgcccaaaagttccagggccgcgtgaccatgactgtggacacctccacctccaccgtgtacatggagctctcaagcctgaggtccgaggatactgccgtgtactattgtgcgcggggggattacgacgaaagctacttcgactactggggccagggcactctggtcaccgtgtcgtcagcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcttatattcaaagctcaccgtggacaagagcaggtggcagcaggggaacgtcttctcatgctccgtgatgcatgaggctctgcacaaccactacacgcagaagagcctctccctgtctcccgggaaa

SEQ ID NO: 191 - DNA, 1347 bp, synthetic construct
  source 1..1347: mol_type = other DNA
caggcttatcttcagcagtctggggctgcgctggtgaggcctggggcctcagtgaagatgtcctgcaagtcttctggctacacatttaccacttacaatatacactgggtaaagcagacacctagacagggcctggaatggattggagctatttttccaggaaatggtcatatttcctacaatcagaagttcaagggcaaggccacactgactgtagacaaatcctccaacacagcctacatgcagctcagcagcctgacatctgaagactctgcggtctatttctgtgcaagaggggattacgacgagtcttactttgactactggggccaaggcaccactctcacagtctcctcatgctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcttatattcaaagctcaccgtggacaagagcaggtggcagcaggggaacgtcttctcatgctccgtgatgcatgaggctctgcacaaccactacacgcagaagagcctctccctgtctcccgggaaa

SEQ ID NO: 192 - DNA, 1347 bp, synthetic construct
  source 1..1347: mol_type = other DNA
caagtgcagcttgtgcagtccggagctgaagtcaagaagcctggtgccagcgtgaaagtgtcctgcaaggcctccggatacaccttcaccacgtacaacatgcattgggtcagacaggcaccgggacagggtctggaatggatgggggccatttttcccggaaacggccacatctcgtacgcccaaaagttccagggccgcgtgaccatgactgtggacacctccacctccaccgtgtacatggagctctcaagcctgaggtccgaggatactgccgtgtactattgtgcgcggggggattacgacgaaagctacttcgactactggggccagggcactctggtcaccgtgtcgtcagcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaggccgccgggggatcctcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagtacaacagcacgtaccgtgtggtcagcgtcctcaccgtcctgcaccaggactggctgaacggcaaggagtacaagtgcaaggtctccaacaaagccctcccggcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcttatattcaaagctcaccgtggacaagagcaggtggcagcaggggaacgtcttctcatgctccgtgatgcatgaggctctgcacaaccactacacgcagaagagcctctccctgtctcccgggaaa

SEQ ID NO: 193 - DNA, 1347 bp, synthetic construct
  source 1..1347: mol_type = other DNA
caagtgcagcttgtgcagtccggagctgaagtcaagaagcctggtgccagcgtgaaagtgtcctgcaaggcctccggatacaccttcaccacgtacaacatgcattgggtcagacaggcaccgggacagggtctggaatggatgggggccatttttcccggaaacggccacatctcgtacgcccaaaagttccagggccgcgtgaccatgactgtggacacctccacctccaccgtgtacatggagctctcaagcctgaggtccgaggatactgccgtgtactattgtgcgcggggggattacgacgaaagctacttcgactactggggccagggcactctggtcaccgtgtcgtcatgctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaggccgccgggggatcctcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagtacaacagcacgtaccgtgtggtcagcgtcctcaccgtcctgcaccaggactggctgaacggcaaggagtacaagtgcaaggtctccaacaaagccctcccggcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcttatattcaaagctcaccgtggacaagagcaggtggcagcaggggaacgtcttctcatgctccgtgatgcatgaggctctgcacaaccactacacgcagaagagcctctccctgtctcccgggaaa

SEQ ID NO: 194 - DNA, 642 bp, synthetic construct
  source 1..642: mol_type = other DNA
gacattcagatgacccaatccccctcatcactgtcggcttccgtgggcgaccgcgtgaccatcacctgtaaagcctccgaggggatctacaacagactcgcctggtatcagcagaagccaggaaaggcccctaagctccttctgtacggagtgatctccctggaaactggagtgccgagccggtttagcggcagcggttccggaactgactacacgttgaccatctcctcgctgcaacccgaagatttcgcgacctactactgccagcagtactggtcgattccgtggactttcggtggcgggaccaaggtcgagatcaaaagaacagtggccgctccgagcgtgttcatctttccaccaagcgacgagcagctgaaaagcggcacagcctctgtcgtgtgcctgctgaacaacttctaccccagagaagccaaggtgcagtggaaggtggacaatgccctgcagagcggcaatagccaagagagcgtgaccgagcaggacagcaaggatagcacctacagcctgtccagcacactgaccctgagcaaggccgactacgagaagcacaaagtgtacgcctgcgaagtgacacaccagggcctgtctagccctgtgaccaagagcttcaaccggggcgagtgt

SEQ ID NO: 195 - DNA, 642 bp, synthetic construct
  source 1..642: mol_type = other DNA
gatattcagatgacacagagccctagcagcctgagcgcctctgtgggcgatagagtgacaatcacatgcaaggccagcgagggcatctacaacagactggcctggtatcagcagaagcccggaaaggctcctaagctgctgctgtacggcgtgatctctctggaaacaggcgtgccaagcagattttctggcagcggctctggcaccgactacaccctgacaatttctagcctgcagtgcgaggacgtggccacctactactgccagcagtactggtctatcccctggacctttggcggaggcaccaaggtggaaatcaagagaacagtggccgctccgagcgtgttcatctttccaccaagcgacgagcagctgaaaagcggcacagcctctgtcgtgtgcctgctgaacaacttctaccccagagaagccaaggtgcagtggaaggtggacaatgccctgcagagcggcaatagccaagagagcgtgaccgagcaggacagcaaggatagcacctacagcctgtccagcacactgaccctgagcaaggccgactacgagaagcacaaagtgtacgcctgcgaagtgacacaccagggcctgtctagccctgtgaccaagagcttcaaccggggcgagtgt

SEQ ID NO: 196 - DNA, 1344 bp, synthetic construct
  source 1..1344: mol_type = other DNA
caagtgcagctggtgcagtctggtgccgaggtcaagaagccaggctccagcgtgaaagtgtcctgcaaggcctcgggatacgcattccggtcgtactggatctcctgggtccgccaggcccccggacaaggccttgaatggatggggcagatctttcctggggacggcgacattaactacgcgcagaagttccagggcagagtgaccatcactgccgataagagcacctccactgcgtacatggaactgagctccctgaggtccgaggataccgccgtgtactattgcgctcggatggacttctactcctggttcccgtactggggacagggtaccctcgtcaccgtgtcatcagcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcttatattcaaagctcaccgtggacaagagcaggtggcagcaggggaacgtcttctcatgctccgtgatgcatgaggctctgcacaaccactacacgcagaagagcctctccctgtctcccgggaaa

SEQ ID NO: 197 - DNA, 1344 bp, synthetic construct
  source 1..1344: mol_type = other DNA
caagtgcagctggtgcagtctggtgccgaggtcaagaagccaggctccagcgtgaaagtgtcctgcaaggcctcgggatacgcattccggtcgtactggatctcctgggtccgccaggcccccggacaaggccttgaatggatggggcagatctttcctggggacggcgacattaactacgcgcagaagttccagggcagagtgaccatcactgccgataagagcacctccactgcgtacatggaactgagctccctgaggtccgaggataccgccgtgtactattgcgctcggatggacttctactcctggttcccgtactggggacagggtaccctcgtcaccgtgtcatcatgctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcttatattcaaagctcaccgtggacaagagcaggtggcagcaggggaacgtcttctcatgctccgtgatgcatgaggctctgcacaaccactacacgcagaagagcctctccctgtctcccgggaaa

SEQ ID NO: 198 - DNA, 1344 bp, synthetic construct
  source 1..1344: mol_type = other DNA
caagtgcagctggtgcagtctggtgccgaggtcaagaagccaggctccagcgtgaaagtgtcctgcaaggcctcgggatacgcattccggtcgtactggatctcctgggtccgccaggcccccggacaaggccttgaatggatggggcagatctttcctggggacggcgacattaactacgcgcagaagttccagggcagagtgaccatcactgccgataagagcacctccactgcgtacatggaactgagctccctgaggtccgaggataccgccgtgtactattgcgctcggatggacttctactcctggttcccgtactggggacagggtaccctcgtcaccgtgtcatcagcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaggccgccgggggatcctcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagtacaacagcacgtaccgtgtggtcagcgtcctcaccgtcctgcaccaggactggctgaacggcaaggagtacaagtgcaaggtctccaacaaagccctcccggcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcttatattcaaagctcaccgtggacaagagcaggtggcagcaggggaacgtcttctcatgctccgtgatgcatgaggctctgcacaaccactacacgcagaagagcctctccctgtctcccgggaaa

SEQ ID NO: 199 - DNA, 1344 bp, synthetic construct
  source 1..1344: mol_type = other DNA
caagtgcagctggtgcagtctggtgccgaggtcaagaagccaggctccagcgtgaaagtgtcctgcaaggcctcgggatacgcattccggtcgtactggatctcctgggtccgccaggcccccggacaaggccttgaatggatggggcagatctttcctggggacggcgacattaactacgcgcagaagttccagggcagagtgaccatcactgccgataagagcacctccactgcgtacatggaactgagctccctgaggtccgaggataccgccgtgtactattgcgctcggatggacttctactcctggttcccgtactggggacagggtaccctcgtcaccgtgtcatcatgctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaggccgccgggggatcctcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagtacaacagcacgtaccgtgtggtcagcgtcctcaccgtcctgcaccaggactggctgaacggcaaggagtacaagtgcaaggtctccaacaaagccctcccggcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcttatattcaaagctcaccgtggacaagagcaggtggcagcaggggaacgtcttctcatgctccgtgatgcatgaggctctgcacaaccactacacgcagaagagcctctccctgtctcccgggaaa

SEQ ID NO: 200 - DNA, 660 bp, synthetic construct
  source 1..660: mol_type = other DNA
gacattgtgatgacccagtcccccgattcccttgccgtgtccctcggtgaacgggccactatcaactgcaaatccagccagtcgctgctgaactcggggaaccagaagaatgatctggcctggtaccagcagaagcctggccagcccccaaagctgctcatctacggagcatcaactcgcgaatccggagtgccggacagattttccgggagcggatcaggcaccgacttcaccttgaccatttcgagcctgcaagcggaggacgtcgctgtgtactactgtcagaacgaccactcctatccgctgactttcggacaaggcaccaagctggagatcaaaagaacagtggccgctccgagcgtgttcatctttccaccaagcgacgagcagctgaaaagcggcacagcctctgtcgtgtgcctgctgaacaacttctaccccagagaagccaaggtgcagtggaaggtggacaatgccctgcagagcggcaatagccaagagagcgtgaccgagcaggacagcaaggatagcacctacagcctgtccagcacactgaccctgagcaaggccgactacgagaagcacaaagtgtacgcctgcgaagtgacacaccagggcctgtctagccctgtgaccaagagcttcaaccggggcgagtgt

SEQ ID NO: 201 - DNA, 660 bp, synthetic construct
  source 1..660: mol_type = other DNA
gacattgtgatgacccagtcccccgattcccttgccgtgtccctcggtgaacgggccactatcaactgcaaatccagccagtcgctgctgaactcggggaaccagaagaatgatctggcctggtaccagcagaagcctggccagcccccaaagctgctcatctacggagcatcaactcgcgaatccggagtgccggacagattttccgggagcggatcaggcaccgacttcaccttgaccatttcgagcctgcaatgcgaggacgtcgctgtgtactactgtcagaacgaccactcctatccgctgactttcggacaaggcaccaagctggagatcaaaagaacagtggccgctccgagcgtgttcatctttccaccaagcgacgagcagctgaaaagcggcacagcctctgtcgtgtgcctgctgaacaacttctaccccagagaagccaaggtgcagtggaaggtggacaatgccctgcagagcggcaatagccaagagagcgtgaccgagcaggacagcaaggatagcacctacagcctgtccagcacactgaccctgagcaaggccgactacgagaagcacaaagtgtacgcctgcgaagtgacacaccagggcctgtctagccctgtgaccaagagcttcaaccggggcgagtgt

SEQ ID NO: 202 - DNA, 1344 bp, synthetic construct
  source 1..1344: mol_type = other DNA
gaagtgcagctggttgaatctggcggaggactgattcagcctggcggctctctgagactgtcttgtgccgcctctggcttcagcctgagcaactactacatcaactgggtccgacaggcccctggcaaaggactggaatggatcggcttcatccctatgtacggcaccacacactacgccaactgggccaagggcagattcaccatcagccgggacaacagcaagaacaccctgtacctgcagatgaacagcctgagagccgaggacaccgccgtgtactattgtgccagaggaatcgccagcatgttctacccttctatctggggccagggcaccctggtcacagttagttctgcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcttatattcaaagctcaccgtggacaagagcaggtggcagcaggggaacgtcttctcatgctccgtgatgcatgaggctctgcacaaccactacacgcagaagagcctctccctgtctcccgggaaa

SEQ ID NO: 203 - DNA, 1344 bp, synthetic construct
  source 1..1344: mol_type = other DNA
gaagtgcagctggttgaatctggcggaggactgattcagcctggcggctctctgagactgtcttgtgccgcctctggcttcagcctgagcaactactacatcaactgggtccgacaggcccctggcaaaggactggaatggatcggcttcatccctatgtacggcaccacacactacgccaactgggccaagggcagattcaccatcagccgggacaacagcaagaacaccctgtacctgcagatgaacagcctgagagccgaggacaccgccgtgtactattgtgccagaggaatcgccagcatgttctacccttctatctggggccagggcaccctggtcacagttagttcttgctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcttatattcaaagctcaccgtggacaagagcaggtggcagcaggggaacgtcttctcatgctccgtgatgcatgaggctctgcacaaccactacacgcagaagagcctctccctgtctcccgggaaa

SEQ ID NO: 204 - DNA, 1344 bp, synthetic construct
  source 1..1344: mol_type = other DNA
gaagtgcagctggttgaatctggcggaggactgattcagcctggcggctctctgagactgtcttgtgccgcctctggcttcagcctgagcaactactacatcaactgggtccgacaggcccctggcaaaggactggaatggatcggcttcatccctatgtacggcaccacacactacgccaactgggccaagggcagattcaccatcagccgggacaacagcaagaacaccctgtacctgcagatgaacagcctgagagccgaggacaccgccgtgtactattgtgccagaggaatcgccagcatgttctacccttctatctggggccagggcaccctggtcacagttagttctgcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaggccgccgggggatcctcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagtacaacagcacgtaccgtgtggtcagcgtcctcaccgtcctgcaccaggactggctgaacggcaaggagtacaagtgcaaggtctccaacaaagccctcccggcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcttatattcaaagctcaccgtggacaagagcaggtggcagcaggggaacgtcttctcatgctccgtgatgcatgaggctctgcacaaccactacacgcagaagagcctctccctgtctcccgggaaa

SEQ ID NO: 205 - DNA, 1344 bp, synthetic construct
  source 1..1344: mol_type = other DNA
gaagtgcagctggttgaatctggcggaggactgattcagcctggcggctctctgagactgtcttgtgccgcctctggcttcagcctgagcaactactacatcaactgggtccgacaggcccctggcaaaggactggaatggatcggcttcatccctatgtacggcaccacacactacgccaactgggccaagggcagattcaccatcagccgggacaacagcaagaacaccctgtacctgcagatgaacagcctgagagccgaggacaccgccgtgtactattgtgccagaggaatcgccagcatgttctacccttctatctggggccagggcaccctggtcacagttagttcttgctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaggccgccgggggatcctcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagtacaacagcacgtaccgtgtggtcagcgtcctcaccgtcctgcaccaggactggctgaacggcaaggagtacaagtgcaaggtctccaacaaagccctcccggcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcttatattcaaagctcaccgtggacaagagcaggtggcagcaggggaacgtcttctcatgctccgtgatgcatgaggctctgcacaaccactacacgcagaagagcctctccctgtctcccgggaaa

SEQ ID NO: 206 - DNA, 645 bp, synthetic construct
  source 1..645: mol_type = other DNA
gacgttgtgatgacacagagccctagcagcctgagcgcctctgtgggcgatagagtgacaatcacctgtcaggccagcgaggacatccacggctatctggcctggtatcagcagaagcctggaaaggcccctaagctgctgatctactacgcctctacactggccagcggcgtgccaagcagattttctggctctggcagcggcaccgacttcaccctgacaatttctagcctccagcctgaggacttcgccacctactattgtctcggagatggcagcaccagcggcatcacatttggccagggcaccaaggtggaaatcaaaagaacagtggccgctccgagcgtgttcatctttccaccaagcgacgagcagctgaaaagcggcacagcctctgtcgtgtgcctgctgaacaacttctaccccagagaagccaaggtgcagtggaaggtggacaatgccctgcagagcggcaatagccaagagagcgtgaccgagcaggacagcaaggatagcacctacagcctgtccagcacactgaccctgagcaaggccgactacgagaagcacaaagtgtacgcctgcgaagtgacacaccagggcctgtctagccctgtgaccaagagcttcaaccggggcgagtgt

SEQ ID NO: 207 - DNA, 645 bp, synthetic construct
  source 1..645: mol_type = other DNA
gacgttgtgatgacacagagccctagcagcctgagcgcctctgtgggcgatagagtgacaatcacctgtcaggccagcgaggacatccacggctatctggcctggtatcagcagaagcctggaaaggcccctaagctgctgatctactacgcctctacactggccagcggcgtgccaagcagattttctggctctggcagcggcaccgacttcaccctgacaatttctagcctccagtgcgaggacgtggccacctactattgtctcggagatggcagcaccagcggcatcacatttggccagggcaccaaggtggaaatcaaaagaacagtggccgctccgagcgtgttcatctttccaccaagcgacgagcagctgaaaagcggcacagcctctgtcgtgtgcctgctgaacaacttctaccccagagaagccaaggtgcagtggaaggtggacaatgccctgcagagcggcaatagccaagagagcgtgaccgagcaggacagcaaggatagcacctacagcctgtccagcacactgaccctgagcaaggccgactacgagaagcacaaagtgtacgcctgcgaagtgacacaccagggcctgtctagccctgtgaccaagagcttcaaccggggcgagtgt

SEQ ID NO: 208 - DNA, 1347 bp, synthetic construct
  source 1..1347: mol_type = other DNA
gaagtgcagctggttgaatctggcggaggactgattcagcctggcggctctctgagactgtcttgtgccgcctctggcatcgatctgagcagctctacaatggcctgggttcgacaggcccctggcaaaggactggaatggatcggcatcattgccagcagcggctctgcctattatgccggatgggccaagggcagattcaccatcagccgggacaacagcaagaacaccctgtacctgcagatgaacagcctgagagccgaggacaccgccgtgtactattgtgttagacaggtgcccggctacaacgacaacagatatgtgtggggccagggcaccctggtcacagttagttctgcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcttatattcaaagctcaccgtggacaagagcaggtggcagcaggggaacgtcttctcatgctccgtgatgcatgaggctctgcacaaccactacacgcagaagagcctctccctgtctcccgggaaa

SEQ ID NO: 209 - DNA, 1347 bp, synthetic construct
  source 1..1347: mol_type = other DNA
gaagtgcagctggttgaatctggcggaggactgattcagcctggcggctctctgagactgtcttgtgccgcctctggcatcgatctgagcagctctacaatggcctgggttcgacaggcccctggcaaaggactggaatggatcggcatcattgccagcagcggctctgcctattatgccggatgggccaagggcagattcaccatcagccgggacaacagcaagaacaccctgtacctgcagatgaacagcctgagagccgaggacaccgccgtgtactattgtgttagacaggtgcccggctacaacgacaacagatatgtgtggggccagggcaccctggtcacagttagttcttgctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcttatattcaaagctcaccgtggacaagagcaggtggcagcaggggaacgtcttctcatgctccgtgatgcatgaggctctgcacaaccactacacgcagaagagcctctccctgtctcccgggaaa

SEQ ID NO: 210 - DNA, 1347 bp, synthetic construct
  source 1..1347: mol_type = other DNA
gaagtgcagctggttgaatctggcggaggactgattcagcctggcggctctctgagactgtcttgtgccgcctctggcatcgatctgagcagctctacaatggcctgggttcgacaggcccctggcaaaggactggaatggatcggcatcattgccagcagcggctctgcctattatgccggatgggccaagggcagattcaccatcagccgggacaacagcaagaacaccctgtacctgcagatgaacagcctgagagccgaggacaccgccgtgtactattgtgttagacaggtgcccggctacaacgacaacagatatgtgtggggccagggcaccctggtcacagttagttctgcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaggccgccgggggatcctcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagtacaacagcacgtaccgtgtggtcagcgtcctcaccgtcctgcaccaggactggctgaacggcaaggagtacaagtgcaaggtctccaacaaagccctcccggcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcttatattcaaagctcaccgtggacaagagcaggtggcagcaggggaacgtcttctcatgctccgtgatgcatgaggctctgcacaaccactacacgcagaagagcctctccctgtctcccgggaaa

SEQ ID NO: 211 - DNA, 1347 bp, synthetic construct
  source 1..1347: mol_type = other DNA
gaagtgcagctggttgaatctggcggaggactgattcagcctggcggctctctgagactgtcttgtgccgcctctggcatcgatctgagcagctctacaatggcctgggttcgacaggcccctggcaaaggactggaatggatcggcatcattgccagcagcggctctgcctattatgccggatgggccaagggcagattcaccatcagccgggacaacagcaagaacaccctgtacctgcagatgaacagcctgagagccgaggacaccgccgtgtactattgtgttagacaggtgcccggctacaacgacaacagatatgtgtggggccagggcaccctggtcacagttagttcttgctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaggccgccgggggatcctcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagtacaacagcacgtaccgtgtggtcagcgtcctcaccgtcctgcaccaggactggctgaacggcaaggagtacaagtgcaaggtctccaacaaagccctcccggcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcttatattcaaagctcaccgtggacaagagcaggtggcagcaggggaacgtcttctcatgctccgtgatgcatgaggctctgcacaaccactacacgcagaagagcctctccctgtctcccgggaaa

SEQ ID NO: 212 - DNA, 651 bp, synthetic construct
  source 1..651: mol_type = other DNA
gctattgtgatgacacagagccctagcagcctgagcgcctctgtgggcgatagagtgacaatcacctgtcaggccagcgagagcgtgtacaacgccaacagctgtagctggtatcagcagaagcctggcaaggcccctaagctgctgatctacgatgcctctgatctggccagcggcgtgccaagcagattttctggctctggcagcggcaccgacttcaccctgacaatttctagcctccagcctgaggacttcgccacctactattgtgccggctacaaaggcaatggcgacgccgcttttggccagggcacaaaggtggaaatcaaaagaacagtggccgctccgagcgtgttcatctttccaccaagcgacgagcagctgaaaagcggcacagcctctgtcgtgtgcctgctgaacaacttctaccccagagaagccaaggtgcagtggaaggtggacaatgccctgcagagcggcaatagccaagagagcgtgaccgagcaggacagcaaggatagcacctacagcctgtccagcacactgaccctgagcaaggccgactacgagaagcacaaagtgtacgcctgcgaagtgacacaccagggcctgtctagccctgtgaccaagagcttcaaccggggcgagtgt

SEQ ID NO: 213 - DNA, 651 bp, synthetic construct
  source 1..651: mol_type = other DNA
gctattgtgatgacacagagccctagcagcctgagcgcctctgtgggcgatagagtgacaatcacctgtcaggccagcgagagcgtgtacaacgccaacagctgtagctggtatcagcagaagcctggcaaggcccctaagctgctgatctacgatgcctctgatctggccagcggcgtgccaagcagattttctggctctggcagcggcaccgacttcaccctgacaatttctagcctccagtgcgaggacgtggccacctactattgtgccggctacaaaggcaatggcgacgccgcttttggccagggcacaaaggtggaaatcaaaagaacagtggccgctccgagcgtgttcatctttccaccaagcgacgagcagctgaaaagcggcacagcctctgtcgtgtgcctgctgaacaacttctaccccagagaagccaaggtgcagtggaaggtggacaatgccctgcagagcggcaatagccaagagagcgtgaccgagcaggacagcaaggatagcacctacagcctgtccagcacactgaccctgagcaaggccgactacgagaagcacaaagtgtacgcctgcgaagtgacacaccagggcctgtctagccctgtgaccaagagcttcaaccggggcgagtgt

SEQ ID NO: 214 - DNA, 354 bp, synthetic construct
  source 1..354: mol_type = other DNA
gaggtgcagctggtggagagcggcggcggcctgatccagcccggcggcagcctgaggctgagctgcgccgccagcggcttcaccctgagcaactactacatcaactgggtgaggcaggcccccggcaagggcctggagtggatcggcttcatccccatgtacggcaccacccactacgccaactgggccaagggcaggttcaccatcagcagggacaacagcaagaacaccctgtacctgcagatgaacagcctgagggccgaggacaccgccgtgtactactgcgccaggggcatcgccagcatgttctaccccagcatctggggccagggcaccctggtgaccgtgagcagc

SEQ ID NO: 215 - DNA, 354 bp, synthetic construct
  source 1..354: mol_type = other DNA
gaggtgcagctggtggagagcggcggcggcctgatccagcccggcggcagcctgaggctgagctgcgccgccagcggcttcagcgtgagcaactactacatcaactgggtgaggcaggcccccggcaagggcctggagtggatcggcttcatccccatgtacggcaccacccactacgccaactgggccaagggcaggttcaccatcagcagggacaacagcaagaacaccctgtacctgcagatgaacagcctgagggccgaggacaccgccgtgtactactgcgccaggggcatcgccagcatgttctaccccagcatctggggccagggcaccctggtgaccgtgagcagc

SEQ ID NO: 216 - DNA, 354 bp, synthetic construct
  source 1..354: mol_type = other DNA
gaggtgcagctggtggagagcggcggcggcctgatccagcccggcggcagcctgaggctgagctgcgccgccagcggcttcaccgtgagcaactactacatcaactgggtgaggcaggcccccggcaagggcctggagtggatcggcttcatccccatgtacggcaccacccactacgccaactgggccaagggcaggttcaccatcagcagggacaacagcaagaacaccctgtacctgcagatgaacagcctgagggccgaggacaccgccgtgtactactgcgccaggggcatcgccagcatgttctaccccagcatctggggccagggcaccctggtgaccgtgagcagc

SEQ ID NO: 217 - DNA, 354 bp, synthetic construct
  source 1..354: mol_type = other DNA
gaggtgcagctggtggagagcggcggcggcctgatccagcccggcggcagcctgaggctgagctgcgccgccagcggcttcagcctgagcaactactacatcaactgggtgaggcaggcccccggcaagggcctggagtgggtgggcttcatccccatgtacggcaccacccactacgccaactgggccaagggcaggttcaccatcagcagggacaacagcaagaacaccctgtacctgcagatgaacagcctgagggccgaggacaccgccgtgtactactgcgccaggggcatcgccagcatgttctaccccagcatctggggccagggcaccctggtgaccgtgagcagc

SEQ ID NO: 218 - DNA, 354 bp, synthetic construct
  source 1..354: mol_type = other DNA
gaggtgcagctggtggagagcggcggcggcctgatccagcccggcggcagcctgaggctgagctgcgccgccagcggcttcagcctgagcaactactacatcaactgggtgaggcaggcccccggcaagggcctggagtggatcggcttcatccccatgtacggcaccacccactacgccgacagcgtgaagggcaggttcaccatcagcagggacaacagcaagaacaccctgtacctgcagatgaacagcctgagggccgaggacaccgccgtgtactactgcgccaggggcatcgccagcatgttctaccccagcatctggggccagggcaccctggtgaccgtgagcagc

SEQ ID NO: 219 - DNA, 354 bp, synthetic construct
  source 1..354: mol_type = other DNA
gaggtgcagctggtggagagcggcggcggcctgatccagcccggcggcagcctgaggctgagctgcgccgccagcggcttcagcctgagcaactactacatcaactgggtgaggcaggcccccggcaagggcctggagtggatcggcttcatccccatgtacggcaccacccactacgccaacagcgtgaagggcaggttcaccatcagcagggacaacagcaagaacaccctgtacctgcagatgaacagcctgagggccgaggacaccgccgtgtactactgcgccaggggcatcgccagcatgttctaccccagcatctggggccagggcaccctggtgaccgtgagcagc

SEQ ID NO: 220 - DNA, 354 bp, synthetic construct
  source 1..354: mol_type = other DNA
gaggtgcagctggtggagagcggcggcggcctgatccagcccggcggcagcctgaggctgagctgcgccgccagcggcttcagcctgagcaactactacatcaactgggtgaggcaggcccccggcaagggcctggagtggatcggcttcatccccatgtacggcaccacccactacgccaactgggtgaagggcaggttcaccatcagcagggacaacagcaagaacaccctgtacctgcagatgaacagcctgagggccgaggacaccgccgtgtactactgcgccaggggcatcgccagcatgttctaccccagcatctggggccagggcaccctggtgaccgtgagcagc

SEQ ID NO: 221 - DNA, 354 bp, synthetic construct
  source 1..354: mol_type = other DNA
gaggtgcagctggtggagagcggcggcggcctgatccagcccggcggcagcctgaggctgagctgcgccgccagcggcttcaccctgagcaactactacatcaactgggtgaggcaggcccccggcaagggcctggagtggatcggcttcatccccatgtacggcaccacccactacgccgacagcgtgaagggcaggttcaccatcagcagggacaacagcaagaacaccctgtacctgcagatgaacagcctgagggccgaggacaccgccgtgtactactgcgccaggggcatcgccagcatgttctaccccagcatctggggccagggcaccctggtgaccgtgagcagc

SEQ ID NO: 222 - DNA, 354 bp, synthetic construct
  source 1..354: mol_type = other DNA
gaggtgcagctggtggagagcggcggcggcctgatccagcccggcggcagcctgaggctgagctgcgccgccagcggcttcaccctgagcaactactacatcaactgggtgaggcaggcccccggcaagggcctggagtggatcggcttcatccccatgtacggcaccacccactacgccaacagcgtgaagggcaggttcaccatcagcagggacaacagcaagaacaccctgtacctgcagatgaacagcctgagggccgaggacaccgccgtgtactactgcgccaggggcatcgccagcatgttctaccccagcatctggggccagggcaccctggtgaccgtgagcagc

SEQ ID NO: 223 - DNA, 354 bp, synthetic construct
  source 1..354: mol_type = other DNA
gaggtgcagctggtggagagcggcggcggcctgatccagcccggcggcagcctgaggctgagctgcgccgccagcggcttcaccctgagcaactactacatcaactgggtgaggcaggcccccggcaagggcctggagtggatcggcttcatccccatgtacggcaccacccactacgccaactgggtgaagggcaggttcaccatcagcagggacaacagcaagaacaccctgtacctgcagatgaacagcctgagggccgaggacaccgccgtgtactactgcgccaggggcatcgccagcatgttctaccccagcatctggggccagggcaccctggtgaccgtgagcagc

SEQ ID NO: 224 - DNA, 354 bp, synthetic construct
  source 1..354: mol_type = other DNA
gaggtgcagctggtggagagcggcggcggcctgatccagcccggcggcagcctgaggctgagctgcgccgccagcggcttcaccgtgagcaactactacatcaactgggtgaggcaggcccccggcaagggcctggagtggatcggcttcatccccatgtacggcaccacccactacgccgacagcgtgaagggcaggttcaccatcagcagggacaacagcaagaacaccctgtacctgcagatgaacagcctgagggccgaggacaccgccgtgtactactgcgccaggggcatcgccagcatgttctaccccagcatctggggccagggcaccctggtgaccgtgagcagc

SEQ ID NO: 225 - DNA, 354 bp, synthetic construct
  source 1..354: mol_type = other DNA
gaggtgcagctggtggagagcggcggcggcctgatccagcccggcggcagcctgaggctgagctgcgccgccagcggcttcaccgtgagcaactactacatcaactgggtgaggcaggcccccggcaagggcctggagtggatcggcttcatccccatgtacggcaccacccactacgccaacagcgtgaagggcaggttcaccatcagcagggacaacagcaagaacaccctgtacctgcagatgaacagcctgagggccgaggacaccgccgtgtactactgcgccaggggcatcgccagcatgttctaccccagcatctggggccagggcaccctggtgaccgtgagcagc

SEQ ID NO: 226 - DNA, 354 bp, synthetic construct
  source 1..354: mol_type = other DNA
gaggtgcagctggtggagagcggcggcggcctgatccagcccggcggcagcctgaggctgagctgcgccgccagcggcttcaccgtgagcaactactacatcaactgggtgaggcaggcccccggcaagggcctggagtggatcggcttcatccccatgtacggcaccacccactacgccaactgggtgaagggcaggttcaccatcagcagggacaacagcaagaacaccctgtacctgcagatgaacagcctgagggccgaggacaccgccgtgtactactgcgccaggggcatcgccagcatgttctaccccagcatctggggccagggcaccctggtgaccgtgagcagc

SEQ ID NO: 227 - DNA, 354 bp, synthetic construct
  source 1..354: mol_type = other DNA
gaggtgcagctggtggagagcggcggcggcctgatccagcccggcggcagcctgaggctgagctgcgccgccagcggcttcaccctgagcaactactacatcaactgggtgaggcaggcccccggcaagggcctggagtgggtgggcttcatccccatgtacggcaccacccactacgccgacagcgtgaagggcaggttcaccatcagcagggacaacagcaagaacaccctgtacctgcagatgaacagcctgagggccgaggacaccgccgtgtactactgcgccaggggcatcgccagcatgttctaccccagcatctggggccagggcaccctggtgaccgtgagcagc

SEQ ID NO: 228 - DNA, 354 bp, synthetic construct
  source 1..354: mol_type = other DNA
gaggtgcagctggtggagagcggcggcggcctgatccagcccggcggcagcctgaggctgagctgcgccgccagcggcttcaccctgagcaactactacatcaactgggtgaggcaggcccccggcaagggcctggagtgggtgggcttcatccccatgtacggcaccacccactacgccaacagcgtgaagggcaggttcaccatcagcagggacaacagcaagaacaccctgtacctgcagatgaacagcctgagggccgaggacaccgccgtgtactactgcgccaggggcatcgccagcatgttctaccccagcatctggggccagggcaccctggtgaccgtgagcagc

SEQ ID NO: 229 - DNA, 354 bp, synthetic construct
  source 1..354: mol_type = other DNA
gaggtgcagctggtggagagcggcggcggcctgatccagcccggcggcagcctgaggctgagctgcgccgccagcggcttcaccctgagcaactactacatcaactgggtgaggcaggcccccggcaagggcctggagtgggtgggcttcatccccatgtacggcaccacccactacgccaactgggtgaagggcaggttcaccatcagcagggacaacagcaagaacaccctgtacctgcagatgaacagcctgagggccgaggacaccgccgtgtactactgcgccaggggcatcgccagcatgttctaccccagcatctggggccagggcaccctggtgaccgtgagcagc

SEQ ID NO: 230 - DNA, 354 bp, synthetic construct
  source 1..354: mol_type = other DNA
gaggtgcagctggtggagagcggcggcggcctgatccagcccggcggcagcctgaggctgagctgcgccgccagcggcttcaccgtgagcaactactacatcaactgggtgaggcaggcccccggcaagggcctggagtgggtgggcttcatccccatgtacggcaccacccactacgccgacagcgtgaagggcaggttcaccatcagcagggacaacagcaagaacaccctgtacctgcagatgaacagcctgagggccgaggacaccgccgtgtactactgcgccaggggcatcgccagcatgttctaccccagcatctggggccagggcaccctggtgaccgtgagcagc

SEQ ID NO: 231 - DNA, 354 bp, synthetic construct
  source 1..354: mol_type = other DNA
gaggtgcagctggtggagagcggcggcggcctgatccagcccggcggcagcctgaggctgagctgcgccgccagcggcttcaccgtgagcaactactacatcaactgggtgaggcaggcccccggcaagggcctggagtgggtgggcttcatccccatgtacggcaccacccactacgccaacagcgtgaagggcaggttcaccatcagcagggacaacagcaagaacaccctgtacctgcagatgaacagcctgagggccgaggacaccgccgtgtactactgcgccaggggcatcgccagcatgttctaccccagcatctggggccagggcaccctggtgaccgtgagcagc

SEQ ID NO: 232 - DNA, 354 bp, synthetic construct
  source 1..354: mol_type = other DNA
gaggtgcagctggtggagagcggcggcggcctgatccagcccggcggcagcctgaggctgagctgcgccgccagcggcttcaccgtgagcaactactacatcaactgggtgaggcaggcccccggcaagggcctggagtgggtgggcttcatccccatgtacggcaccacccactacgccaactgggtgaagggcaggttcaccatcagcagggacaacagcaagaacaccctgtacctgcagatgaacagcctgagggccgaggacaccgccgtgtactactgcgccaggggcatcgccagcatgttctaccccagcatctggggccagggcaccctggtgaccgtgagcagc

SEQ ID NO: 233 - DNA, 324 bp, synthetic construct
  source 1..324: mol_type = other DNA
gacgtggtgatgacccagagccccagcagcctgagcgccagcgtgggcgacagggtgaccatcacctgccaggccagcgaggacatccacggctacctgaactggtaccagcagaagcccggcaaggcccccaagctgctgatctactacgccagcaccctggccagcggcgtgcccagcaggttcagcggcagcggcagcggcaccgacttcaccctgaccatcagcagcctgcagcccgaggacttcgccacctactactgcctgggcgacggcagcaccagcggcatcaccttcggccagggcaccaaggtggagatcaag

SEQ ID NO: 234 - DNA, 324 bp, synthetic construct
  source 1..324: mol_type = other DNA
gacgtggtgatgacccagagccccagcagcctgagcgccagcgtgggcgacagggtgaccatcacctgccaggccagcgaggacatccacggctacctggcctggtaccagcagaagcccggcaaggcccccaagctgctgatctactacgccagcaccctgcagagcggcgtgcccagcaggttcagcggcagcggcagcggcaccgacttcaccctgaccatcagcagcctgcagcccgaggacttcgccacctactactgcctgggcgacggcagcaccagcggcatcaccttcggccagggcaccaaggtggagatcaag

SEQ ID NO: 235 - DNA, 324 bp, synthetic construct
  source 1..324: mol_type = other DNA
gacgtggtgatgacccagagccccagcagcctgagcgccagcgtgggcgacagggtgaccatcacctgccaggccagcgaggacatccacggctacctgaactggtaccagcagaagcccggcaaggcccccaagctgctgatctactacgccagcaccctgcagagcggcgtgcccagcaggttcagcggcagcggcagcggcaccgacttcaccctgaccatcagcagcctgcagcccgaggacttcgccacctactactgcctgggcgacggcagcaccagcggcatcaccttcggccagggcaccaaggtggagatcaag

SEQ ID NO: 236 - DNA, 324 bp, synthetic construct
  source 1..324: mol_type = other DNA
gacgtggtgatgacccagagccccagcagcctgagcgccagcgtgggcgacagggtgaccatcacctgcagggccagcgaggacatccacggctacctggcctggtaccagcagaagcccggcaaggcccccaagctgctgatctactacgccagcaccctggccagcggcgtgcccagcaggttcagcggcagcggcagcggcaccgacttcaccctgaccatcagcagcctgcagcccgaggacttcgccacctactactgcctgggcgacggcagcaccagcggcatcaccttcggccagggcaccaaggtggagatcaag

SEQ ID NO: 237 - DNA, 324 bp, synthetic construct
  source 1..324: mol_type = other DNA
gacgtggtgatgacccagagccccagcagcctgagcgccagcgtgggcgacagggtgaccatcacctgcagggccagcgaggacatccacggctacctgaactggtaccagcagaagcccggcaaggcccccaagctgctgatctactacgccagcaccctggccagcggcgtgcccagcaggttcagcggcagcggcagcggcaccgacttcaccctgaccatcagcagcctgcagcccgaggacttcgccacctactactgcctgggcgacggcagcaccagcggcatcaccttcggccagggcaccaaggtggagatcaag

SEQ ID NO: 238 - DNA, 324 bp, synthetic construct
  source 1..324: mol_type = other DNA
gacgtggtgatgacccagagccccagcagcctgagcgccagcgtgggcgacagggtgaccatcacctgcagggccagcgaggacatccacggctacctggcctggtaccagcagaagcccggcaaggcccccaagctgctgatctactacgccagcaccctgcagagcggcgtgcccagcaggttcagcggcagcggcagcggcaccgacttcaccctgaccatcagcagcctgcagcccgaggacttcgccacctactactgcctgggcgacggcagcaccagcggcatcaccttcggccagggcaccaaggtggagatcaag

SEQ ID NO: 239 - DNA, 324 bp, synthetic construct
  source 1..324: mol_type = other DNA
gacgtggtgatgacccagagccccagcagcctgagcgccagcgtgggcgacagggtgaccatcacctgcagggccagcgaggacatccacggctacctgaactggtaccagcagaagcccggcaaggcccccaagctgctgatctactacgccagcaccctgcagagcggcgtgcccagcaggttcagcggcagcggcagcggcaccgacttcaccctgaccatcagcagcctgcagcccgaggacttcgccacctactactgcctgggcgacggcagcaccagcggcatcaccttcggccagggcaccaaggtggagatcaag

SEQ ID NO: 240 - protein, 105 aa, Homo sapiens
GGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 241 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 242 - protein, 223 aa, Homo sapiens
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 243 - protein, 379 aa, Homo sapiens
MPHSSLHPSIPCPRGHGAQKAALVLLSACLVTLWGLGEPPEHTLRYLVLHLASLQLGLLLNGVCSLAEELRHIHSRYRGSYWRTVRACLGCPLRRGALLLLSIYFYYSLPNAVGPPFTWMLALLGLSQALNILLGLKGLAPAEISAVCEKGNFNVAHGLAWSYYIGYLRLILPELQARIRTYNQHYNNLLRGAVSQRLYILLPLDCGVPDNLSMADPNIRFLDKLPQQTGDHAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 244 - protein, 4 aa, synthetic construct
GGFG

SEQ ID NO: 245 - protein, 4 aa, synthetic construct
GFLG

SEQ ID NO: 246 - protein, 4 aa, synthetic construct
ALAL

SEQ ID NO: 247 - protein, 4 aa, synthetic construct
GGFG

SEQ ID NO: 248 - protein, 574 aa, Homo sapiens
MCPRAARAPATLLLALGAVLWPAAGAWELTILHTNDVHSRLEQTSEDSSKCVNASRCMGGVARLFTKVQQIRRAEPNVLLLDAGDQYQGTIWFTVYKGAEVAHFMNALRYDAMALGNHEFDNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGHSGFEMDKLIAQKVRGVDVVVGGHSNTFLYTGNPPSKEVPAGKYPFIVTSDDGRKVPVVQAYAFGKYLGYLKIEFDERGNVISSHGNPILLNSSIPEDPSIKADINKWRIKLDNYSTQELGKTIVYLDGSSQSCRFRECNMGNLICDAMINNNLRHTDEMFWNHVSMCILNGGGIRSPIDERNNGTITWENLAAVLPFGGTFDLVQLKGSTLKKAFEHSVHRYGQSTGEFLQVGGIHVVYDLSRKPGDRVVKLDVLCTKCRVPSYDPLKMDEVYKVILPNFLANGGDGFQMIKDELLRHDSGDQDINVVSTYISKMKVIYPAVEGRIKFSTGSHCHGSFSLIFLSLWAVIFVLYQ

SEQ ID NO: 249 - protein, 4 aa, synthetic construct
GGFG

SEQ ID NO: 250 - protein, 4 aa, synthetic construct
GGFG

SEQ ID NO: 251 - protein, 4 aa, synthetic construct
GGFG

SEQ ID NO: 252 - protein, 4 aa, synthetic construct
GGFG

SEQ ID NO: 253 - protein, 4 aa, synthetic construct
GFFG

SEQ ID NO: 254 - DNA, 25 bp, synthetic construct
  source 1..25: mol_type = other DNA
ttttttttttttttttttttttttt

SEQ ID NO: 255 - protein, 119 aa, synthetic construct
QAYLQQSGAALVRPGASVKMSCKSSGYTFTTYNIHWVKQTPRQGLEWIGAIFPGNGHISYNQKFKGKATLTVDKSSNTAYMQLSSLTSEDSAVYFCARGDYDESYFDYWGQGTTLTVSS

SEQ ID NO: 256 - protein, 98 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAR

SEQ ID NO: 257 - protein, 119 aa, synthetic construct
QVQLVQSGAEVKKPGASVKVSCKASGYTFTTYNIHWVRQAPGQGLEWMGAIFPGNGHISYNQKFKGRVTMTRDTSTSTVYMELSSLRSEDTAVYYCARGDYDESYFDYWGQGTLVTVSS

SEQ ID NO: 258 - protein, 119 aa, synthetic construct
QVQLVQSGAEVKKPGASVKVSCKASGYTFTTYNMHWVRQAPGQGLEWMGAIFPGNGHISYAQKFQGRVTMTRDTSTSTVYMELSSLRSEDTAVYYCARGDYDESYFDYWGQGTLVTVSS

SEQ ID NO: 259 - protein, 119 aa, synthetic construct
QVQLVQSGAEVKKPGASVKVSCKASGYTFTTYNIHWVRQAPGQGLEWMGAIFPGNGHISYNQKFKGRVTMTVDTSTSTVYMELSSLRSEDTAVYYCARGDYDESYFDYWGQGTLVTVSS

SEQ ID NO: 260 - protein, 107 aa, synthetic construct
DIQMTQSSSSFSVSLGDRVTLSCKASEGIYNRLAWYQQKPGNAPRLLISGVISLETGVPSRFSGSGSGKDYTLSITSLQTEDVATYYCQQYWSIPWTFGGGNKLEIN

SEQ ID NO: 261 - protein, 88 aa, synthetic construct
DIQMTQSPSSLSASVGDRVTITCRASQGISNSLAWYQQKPGKAPKLLLYAASRLESGVPSRFSGSGSGTDYTLTISSLQPEDFATYYC

SEQ ID NO: 262 - protein, 107 aa, synthetic construct
DIQMTQSPSSLSASVGDRVTITCKASEGIYNRLAWYQQKPGKAPKLLLSGVISLETGVPSRFSGSGSGTDYTLTISSLQPEDFATYYCQQYWSIPWTFGGGTKVEIK

SEQ ID NO: 263 - protein, 107 aa, synthetic construct
DIQMTQSPSSLSASVGDRVTITCKASEGIYNRLAWYQQKPGKAPKLLLYGVISLETGVPSRFSGSGSGKDYTLTISSLQPEDFATYYCQQYWSIPWTFGGGTKVEIK

SEQ ID NO: 264 - protein, 107 aa, synthetic construct
DIQMTQSPSSLSASVGDRVTITCKASEGIYNRLAWYQQKPGKAPKLLLSGVISLETGVPSRFSGSGSGKDYTLTISSLQPEDFATYYCQQYWSIPWTFGGGTKVEIK

SEQ ID NO: 265 - protein, 98 aa, synthetic construct
QVQLVQSGAEVKKPGSSVKVSCKASGGTFSSYTISWVRQAPGQGLEWMGRIIPILGTANYAQKFQGRVTITADKSTSTAYMELSSLRSEDTAVYYCAR

SEQ ID NO: 266 - protein, 118 aa, synthetic construct
QVQLVQSGAEVKKPGSSVKVSCKASGYAFRSYWVNWVRQAPGQGLEWMGQIFPGDGDINYSGSFKGRVTITADKSTSTAYMELSSLRSEDTAVYYCARMDFYSWFPYWGQGTLVTVSS

SEQ ID NO: 267 - protein, 118 aa, synthetic construct
QVQLVQSGAEVKKPGSSVKVSCKASGYAFRSYWISWVRQAPGQGLEWMGQIFPGDGDINYAQKFQGRVTITADKSTSTAYMELSSLRSEDTAVYYCARMDFYSWFPYWGQGTLVTVSS

SEQ ID NO: 268 - protein, 113 aa, synthetic construct
DIVMTQSPSSLSVSAGEKVTMSCKSSQSLLNSGNQKNDLAWFQQKPGQPPKLLIYGASTRESGVPDRFTGSGSGTDFTLTISSVQAEDLAVYYCQNDHSYPLTFGTGTKLELK

SEQ ID NO: 269 - protein, 94 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYC

SEQ ID NO: 270 - protein, 113 aa, synthetic construct
DIVMTQSPDSLAVSLGERATINCKSSQSLLNSGNQKNDLAWFQQKPGQPPKLLIYGASTRESGVPDRFSGSGSGTDFTLTISSLQAEDVAVYYCQNDHSYPLTFGQGTKLEIK

SEQ ID NO: 271 - protein, 115 aa, synthetic construct; identical to SEQ ID NO: 55

SEQ ID NO: 272 - protein, 97 aa, synthetic construct
EVQLVESGGGLIQPGGSLRLSCAASGFTVSSNYMSWVRQAPGKGLEWVSVIYSGGSTYYADSVKGRFTISRDNSKNTLYLQMNSLRAEDTAVYYCAR

SEQ ID NO: 273 - protein, 118 aa, synthetic construct
EVQLVESGGGLIQPGGSLRLSCAASGFSLSNYYINWVRQAPGKGLEWIGFIPMYGTTHYANWAKGRFTISRDNSKNTLYLQMNSLRAEDTAVYYCARGIASMFYPSIWGQGTLVTVSS

SEQ ID NO: 274 - protein, 108 aa, synthetic construct
DVVMTQTPSSTSAAVGGAVTIKCQASEDIHGYLAWYQQKPGQPPKLLIYYASTLASGVSSRFKGSGSGTEYTLTISGVQREDAATYYCLGDGSTSGITFGGGTEFEIL

SEQ ID NO: 275 - protein, 88 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYC

SEQ ID NO: 276 - protein, 117 aa, synthetic construct
QSVEESGGRLVTPGTPLTLTCTVSGIDLSSSTMAWVRQAPGKGLEYIGIIASSGSAYYAGWAKGRFTISKTSSTTVDLKITSPTTEDTATYFCVRQVPGYNDNRYVWGPGTLVTVSS

SEQ ID NO: 277 - protein, 97 aa, synthetic construct
EVQVVESGGGLIQPGGSLRLSCAASGFTVSSNYMSWVRQAPGKGLEWVSVIYSGGSTYYADSVKGRFTISRDNSKNTLYLQMNSLRAEDTAVYYCAR

SEQ ID NO: 278 - protein, 110 aa, synthetic construct
AIVMTQTPSSVSAAVGGTVTIHCQASESVYNANSCSWFQQKPGQRPKLLIYDASDLASGVPSRFRGSGSGTQFTLTINNVQREDVATYFCAGYKGNGDAAFGGGTELEIL

SEQ ID NO: 279 - protein, 88 aa, synthetic construct; identical to SEQ ID NO: 275

SEQ ID NO: 280 - protein, 449 aa, synthetic construct
QAYLQQSGAALVRPGASVKMSCKSSGYTFTTYNIHWVKQTPRQGLEWIGAIFPGNGHISYNQKFKGKATLTVDKSSNTAYMQLSSLTSEDSAVYFCARGDYDESYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 281 - protein, 118 aa, synthetic construct
EVQLVESGGGLIQPGGSLRLSCAASGFSVSNYYINWVRQAPGKGLEWIGFIPMYGTTHYANWAKGRFTISRDNSKNTLYLQMNSLRAEDTAVYYCARGIASMFYPSIWGQGTLVTVSS

SEQ ID NO: 282 - protein, 118 aa, synthetic construct
EVQLVESGGGLIQPGGSLRLSCAASGFTVSNYYINWVRQAPGKGLEWIGFIPMYGTTHYANWAKGRFTISRDNSKNTLYLQMNSLRAEDTAVYYCARGIASMFYPSIWGQGTLVTVSS

SEQ ID NO: 283 - protein, 118 aa, synthetic construct
EVQLVESGGGLIQPGGSLRLSCAASGFSLSNYYINWVRQAPGKGLEWVGFIPMYGTTHYANWAKGRFTISRDNSKNTLYLQMNSLRAEDTAVYYCARGIASMFYPSIWGQGTLVTVSS

SEQ ID NO: 284 - protein, 118 aa, synthetic construct
EVQLVESGGGLIQPGGSLRLSCAASGFSLSNYYINWVRQAPGKGLEWIGFIPMYGTTHYADSVKGRFTISRDNSKNTLYLQMNSLRAEDTAVYYCARGIASMFYPSIWGQGTLVTVSS

SEQ ID NO: 285 - protein, 118 aa, synthetic construct
EVQLVESGGGLIQPGGSLRLSCAASGFSLSNYYINWVRQAPGKGLEWIGFIPMYGTTHYANSVKGRFTISRDNSKNTLYLQMNSLRAEDTAVYYCARGIASMFYPSIWGQGTLVTVSS

SEQ ID NO: 286 - protein, 118 aa, synthetic construct
EVQLVESGGGLIQPGGSLRLSCAASGFSLSNYYINWVRQAPGKGLEWIGFIPMYGTTHYANWVKGRFTISRDNSKNTLYLQMNSLRAEDTAVYYCARGIASMFYPSIWGQGTLVTVSS

SEQ ID NO: 287 - protein, 118 aa, synthetic construct
EVQLVESGGGLIQPGGSLRLSCAASGFTLSNYYINWVRQAPGKGLEWIGFIPMYGTTHYADSVKGRFTISRDNSKNTLYLQMNSLRAEDTAVYYCARGIASMFYPSIWGQGTLVTVSS

SEQ ID NO: 288 - DNA, 36 bp, synthetic construct
  source 1..36: mol_type = other DNA
cagtcgctgctgaactcggggaaccagaagaatgat

(19) United States
(12) Patent Application Publication
(10) Pub. No.: US 2026/0139068 A1
(43) Pub. Date: May 21, 2026

(54) ANTI-CD73 ANTIBODIES, CONJUGATES, AND METHODS OF USE

(71) Applicant: EISAI R&D MANAGEMENT CO., LTD., Tokyo (JP)

(72) Inventors: Jared SPIDEL, Downington, PA (US); Kenzo ARAI, Tsukuba-shi (JP); Xin CHENG, Nutley, NJ (US); Keiji FURUUCHI, Nutley, NJ (US); Earl F. ALBONE, Collegeville, PA (US); Yosuke KABURAGI, Tsukuba-shi (JP); Dae-Shik KIM, Cambridge, MA (US); Yoshinobu YAMANE, Tsukuba-shi (JP)

(21) Appl. No.: 19/305,651

(22) Filed: Aug. 20, 2025

Related U.S. Application Data
(60) Provisional application No. 63/685,563, filed on Aug. 21, 2024.

(51) Int. Cl. C07K 16/28; A61K 47/68; A61P 35/00

(52) U.S. Cl. CPC C07K 16/2896; A61K 47/6803; A61K 47/6849; A61P 35/00; C07K 2317/24; C07K 2317/76; C07K 2317/77; C07K 2317/94

(57) ABSTRACT

Antibodies, antigen-binding fragments, and conjugates (e.g., antibody-drug conjugates) thereof that bind CD73 are disclosed. The disclosure further relates to methods and compositions for use in the treatment of, e.g., cancer by administering the compositions provided herein.

This application claims the benefit of U.S. Provisional Application No. 63/685,563, filed on Aug. 21, 2024, the contents of which are incorporated by reference in their entirety.

The present application contains a Sequence Listing which has been submitted electronically in ST.26 XML format and is hereby incorporated by reference in its entirety. Said ST.26 copy, created on Dec. 18, 2025, is named 15648-0056-00000.xml and is 311,370 bytes in size.

The present disclosure relates to anti-CD73 antibodies and antigen-binding fragments thereof, as well as conjugates such as antibody-drug conjugates (ADCs), e.g., those comprising a STING agonist, and their use in the treatment and diagnosis of cancers that express CD73 and/or are amenable to treatment by modulating STING pathway activity or by administering a composition disclosed herein.

Cancer is among the leading causes of morbidity and mortality worldwide, with approximately 20 million new cases and 9.7 million cancer-related deaths in 2022. The most common causes of cancer death are cancers of: lung (1.8 million deaths); colorectal cancer (900,000 deaths); liver (760,000 deaths); breast (670,000 deaths); and stomach (660,000 deaths). The number of new cancer cases is expected to rise by about 77% to approximately 35 million new cancer cases per year in 2050. See, e.g., International Agency for Research on Cancer GLOBOCAN 2022.

CD73 is a cell surface ecto-5′-nucleotidase which converts extracellular adenosine monophosphate to adenosine. As one of the key enzymes involved in the generation of immune suppressive adenosine, CD73 has been implicated in the regulation of various cancer-associated processes, including proliferation, adhesion, migration, metastasis, and inhibition of the anti-tumor response. See, e.g., Bach et al. (2023) Int. J. Mol. Sci. 24(14): 11759. Researchers have demonstrated that CD73 is overexpressed in a variety of epithelial carcinomas, including, but not limited to, glioblastoma, thyroid carcinoma, sarcoma, pancreatic carcinoma, stomach adenocarcinoma, colorectal carcinoma, renal cell carcinoma, esophageal carcinoma, thymoma, rectum adenocarcinoma, lung adenocarcinoma, non-small cell lung cancer, and acute myeloid leukaemia thus making it an attractive target for antibody-based cancer therapy. Furthermore, CD73 expression has been associated with decreased patient survival.

In addition to its role in the generation of adenosine, CD73 expression has also been implicated in the epithelial-to-mesenchymal transition (EMT) process. EMT is a mechanism by which cells achieve phenotypic plasticity, and the process is closely associated with cancer metastasis and recurrence. See, e.g., He et al. (2017) Mol. Cancer. 16:63. EMT is thought to be a key process by which cancer cells gain motility and the ability to migrate and invade other sites in the body. Studies have demonstrated that CD73 is directly regulated by hypoxia-inducible factor-1 alpha (HIF-1α), which is known to induce EMT transcription factors. See, e.g., Synnestvedt et al. (2002) J Clin Invest. 110(7):993-1002. SNAI1, a key EMT transcription factor, has been shown to induce CD73 expression in human triple-negative breast cancer cells. See, e.g., Hasmim et al. (2022) Front Immunol. 12:982821. CD73 has also been shown to promote EMT in certain cancers through additional pro-oncogenic signaling axes, such as PI3K-AKT. See, e.g., Ma et al. (2019) J. Hematol. Oncol. 12(1):37. Indeed, CD73 overexpression was shown to increase metastasis-conditioning features (e.g., invasion, migration, and adhesion to the extracellular matrix), and inhibition of CD73 prevented these features. See, e.g., Wang et al. (2008) J. Cancer Res. Clin. Oncol. 134(3):365-72. Without being bound by theory, it is believed that inhibition of CD73, e.g., binding by an anti-CD73 antibody, antigen-binding fragment, and/or ADC, may reduce or prevent EMT in cancer cells.

Given the high expression of CD73 in various cancer types, its role in promoting metastasis, and its association with poor clinical outcome, CD73 is an attractive target for tumor antigen-specific drug delivery approaches, e.g., an antibody-mediated approach. Antibodies conjugated with cytotoxic compounds such as chemotherapeutics have also been explored to enhance the cell-killing activity of antibody-based drug delivery to tumor cells. Nevertheless, a need remains to provide suitable antibodies and/or ADCs that offer a combination of efficient tumor targeting, on-target effects, and/or reduced off-target effects.

STING (stimulator of interferon genes) is a pattern-recognition receptor that senses cyclic dinucleotides in the cytosol and induces the expression of interferons and other inflammatory cytokines (e.g., interferon-β (IFN-β), tumor necrosis factor alpha (TNFα), C-X-C Motif Chemokine Ligand 10 (CXCL10), interleukin-6 (IL-6)), which in turn mediate the innate immune response to infections or diseases, e.g., cancer. STING signaling has been shown to have antitumor effects such as modulation of the vasculature and augmentation of adaptive immunity. STING agonists, e.g., cyclic dinucleotides, often require intra-tumoral injection and show only modest systemic efficacy. These STING agonists are also poorly membrane permeable, which may limit their ability to engage STING inside the cell.

While uses of STING agonists for treating infection or disease have been reported in the art, there remains an unmet need for improved delivery systems that would allow for systemic administration of STING agonists that specifically target tumor sites, including by targeted delivery to tumor-expressed antigens such as CD73. Likewise, there remains a need in the art for improved antibodies that bind CD73 with superior properties, e.g., with respect to antigen-binding and/or the ability to effectively deliver payloads such as a STING agonist to a target cell or tissue expressing CD73.

### SUMMARY OF THE INVENTION

In various embodiments, the present disclosure provides, in part, novel antibodies and antigen-binding fragments that are capable of specifically binding CD73 and may be used alone or linked to one or more additional agents (e.g., as ADCs) and administered as part of pharmaceutical compositions. In some embodiments, the antibodies, antigen-binding fragments, and/or ADCs of the present disclosure may be used to slow, inhibit, and/or reverse tumor growth in mammals, and may be useful for treating human cancer patients.

The present disclosure more specifically relates, in various embodiments, to antibodies and antibody-drug conjugate compounds that are capable of binding and/or killing CD73-expressing cells. In various embodiments, the compounds are also capable of internalizing into a target CD73-expressing cell after binding. ADC compounds comprising a linker that attaches a drug moiety to an antibody moiety are disclosed. An antibody moiety may be a full-length antibody or an antigen-binding fragment.

In various embodiments, the present disclosure provides an internalizing anti-CD73 antibody or internalizing antigen-binding fragment thereof, wherein the antibody or antigen-binding fragment binds specifically to human CD73, and wherein the antibody or antigen-binding fragment comprises three heavy chain complementarity determining regions (HCDR1, HCDR2, and HCDR3) and three light chain complementarity determining regions (LCDR1, LCDR2, and LCDR3), wherein (i) the HCDR1 comprises an amino acid sequence of SEQ ID NO: 1, the HCDR2 comprises an amino acid sequence of SEQ ID NO: 2, the HCDR3 comprises an amino acid sequence of SEQ ID NO: 3, the LCDR1 comprises an amino acid sequence of SEQ ID NO: 4, the LCDR2 comprises an amino acid sequence of SEQ ID NO: 5, and the LCDR3 comprises an amino acid sequence of SEQ ID NO: 6, as defined by the Kabat numbering system; or (ii) the HCDR1 comprises an amino acid sequence of SEQ ID NO: 7, the HCDR2 comprises an amino acid sequence of SEQ ID NO: 8, the HCDR3 comprises an amino acid sequence of SEQ ID NO: 9, the LCDR1 comprises an amino acid sequence of SEQ ID NO: 10, the LCDR2 comprises an amino acid sequence of SEQ ID NO: 11, and the LCDR3 comprises an amino acid sequence of SEQ ID NO: 12, as defined by the IMGT numbering system; or (iii) the HCDR1 comprises an amino acid sequence of SEQ ID NO: 13, the HCDR2 comprises an amino acid sequence of SEQ ID NO: 14, the HCDR3 comprises an amino acid sequence of SEQ ID NO: 15, the LCDR1 comprises an amino acid sequence of SEQ ID NO: 16, the LCDR2 comprises an amino acid sequence of SEQ ID NO: 17, and the LCDR3 comprises an amino acid sequence of SEQ ID NO: 18, as defined by the Kabat numbering system; (iv) the HCDR1 comprises an amino acid sequence of SEQ ID NO: 19, the HCDR2 comprises an amino acid sequence of SEQ ID NO: 20, the HCDR3 comprises an amino acid sequence of SEQ ID NO: 21, the LCDR1 comprises an amino acid sequence of SEQ ID NO: 22, the LCDR2 comprises an amino acid sequence of SEQ ID NO: 23, and the LCDR3 comprises an amino acid sequence of SEQ ID NO: 24, as defined by the IMGT numbering system; (v) the HCDR1 comprises an amino acid sequence of SEQ ID NO: 25, the HCDR2 comprises an amino acid sequence of SEQ ID NO: 26, the HCDR3 comprises an amino acid sequence of SEQ ID NO: 27, the LCDR1 comprises an amino acid sequence of SEQ ID NO: 28, the LCDR2 comprises an amino acid sequence of SEQ ID NO: 29, and the LCDR3 comprises an amino acid sequence of SEQ ID NO: 30, as defined by the Kabat numbering system; (vi) the HCDR1 comprises an amino acid sequence of SEQ ID NO: 31, the HCDR2 comprises an amino acid sequence of SEQ ID NO: 32, the HCDR3 comprises an amino acid sequence of SEQ ID NO: 33, the LCDR1 comprises an amino acid sequence of SEQ ID NO: 34, the LCDR2 comprises an amino acid sequence of SEQ ID NO: 35, and the LCDR3 comprises an amino acid sequence of SEQ ID NO: 36, as defined by the IMGT numbering system; (vii) the HCDR1 comprises an amino acid sequence of SEQ ID NO: 37, the HCDR2 comprises an amino acid sequence of SEQ ID NO: 38, the HCDR3 comprises an amino acid sequence of SEQ ID NO: 39, the LCDR1 comprises an amino acid sequence of SEQ ID NO: 40, the LCDR2 comprises an amino acid sequence of SEQ ID NO: 41, and the LCDR3 comprises an amino acid sequence of SEQ ID NO: 42, as defined by the Kabat numbering system; or (viii) the HCDR1 comprises an amino acid sequence of SEQ ID NO: 43, the HCDR2 comprises an amino acid sequence of SEQ ID NO: 44, the HCDR3 comprises an amino acid sequence of SEQ ID NO: 45, the LCDR1 comprises an amino acid sequence of SEQ ID NO: 46, the LCDR2 comprises an amino acid sequence of SEQ ID NO: 47, and the LCDR3 comprises an amino acid sequence of SEQ ID NO: 48, as defined by the IMGT numbering system.

In some embodiments, the anti-CD73 antibody or antigen-binding fragment comprises three HCDRs comprising amino acid sequences of SEQ ID NO: 1 (HCDR1), SEQ ID NO: 2 (HCDR2), and SEQ ID NO: 3 (HCDR3); and three LCDRs comprising amino acid sequences of SEQ ID NO: 4 (LCDR1), SEQ ID NO: 5 (LCDR2), and SEQ ID NO: 6 (LCDR3), as defined by the Kabat numbering system. In some embodiments, the anti-CD73 antibody or antigen-binding fragment comprises three HCDRs comprising amino acid sequences of SEQ ID NO: 7 (HCDR1), SEQ ID NO: 8 (HCDR2), and SEQ ID NO: 9 (HCDR3); and three LCDRs comprising amino acid sequences of SEQ ID NO: 10 (LCDR1), SEQ ID NO: 11 (LCDR2), and SEQ ID NO: 12 (LCDR3), as defined by the IMGT numbering system. In some embodiments, the anti-CD73 antibody or antigen-binding fragment comprises three HCDRs comprising amino acid sequences of SEQ ID NO: 13 (HCDR1), SEQ ID NO: 14 (HCDR2), and SEQ ID NO: 15 (HCDR3); and three LCDRs comprising amino acid sequences of SEQ ID NO: 16 (LCDR1), SEQ ID NO: 17 (LCDR2), and SEQ ID NO: 18 (LCDR3), as defined by the Kabat numbering system. In some embodiments, the anti-CD73 antibody or antigen-binding fragment comprises three HCDRs comprising amino acid sequences of SEQ ID NO: 19 (HCDR1), SEQ ID NO: 20 (HCDR2), and SEQ ID NO: 21 (HCDR3); and three LCDRs comprising amino acid sequences of SEQ ID NO: 22 (LCDR1), SEQ ID NO: 23 (LCDR2), and SEQ ID NO: 24 (LCDR3), as defined by the IMGT numbering system.

In some embodiments, the anti-CD73 antibody or antigen-binding fragment comprises a heavy chain variable region comprising an amino acid sequence of SEQ ID NO: 49, and a light chain variable region comprising an amino acid sequence selected from SEQ ID NO: 50 or 51. In some embodiments, the anti-CD73 antibody or antigen-binding fragment comprises a heavy chain variable region comprising an amino acid sequence of SEQ ID NO: 52, and a light chain variable region comprising an amino acid sequence selected from SEQ ID NO: 53 or 54. In some embodiments, the anti-CD73 antibody or antigen-binding fragment comprises a heavy chain variable region comprising an amino acid sequence of SEQ ID NO: 55, and a light chain variable region comprising an amino acid sequence selected from SEQ ID NO: 56 or 57. In some embodiments, the anti-CD73 antibody or antigen-binding fragment comprises a heavy chain variable region comprising an amino acid sequence of SEQ ID NO: 58, and a light chain variable region comprising an amino acid sequence selected from SEQ ID NO: 59 or 60.

In some embodiments, the anti-CD73 antibody or antigen-binding fragment comprises a heavy chain variable region comprising an amino acid sequence of SEQ ID NO: 49, and a light chain variable region comprising an amino acid sequence of SEQ ID NO: 50. In some embodiments, the anti-CD73 antibody or antigen-binding fragment comprises a heavy chain variable region comprising an amino acid sequence of SEQ ID NO: 49, and a light chain variable region comprising an amino acid sequence of SEQ ID NO: 51.

In some embodiments, the anti-CD73 antibody or antigen-binding fragment comprises a heavy chain variable region comprising an amino acid sequence of SEQ ID NO: 52, and a light chain variable region comprising an amino acid sequence of SEQ ID NO: 53. In some embodiments, the anti-CD73 antibody or antigen-binding fragment comprises a heavy chain variable region comprising an amino acid sequence of SEQ ID NO: 52, and a light chain variable region comprising an amino acid sequence of SEQ ID NO: 54.

In some embodiments, the anti-CD73 antibody or antigen-binding fragment comprises a human IgG heavy chain constant region. In some embodiments, the anti-CD73 antibody or antigen-binding fragment comprises a human IgG1, IgG2, or IgG4 heavy chain constant region. In some embodiments, the anti-CD73 antibody or antigen-binding fragment comprises a human Ig kappa light chain constant region.

In some embodiments, the IgG1 heavy chain constant region comprises an Fc domain mutated to reduce binding to a Fcγ receptor (FcγR) as compared to an IgG1 Fc-containing antibody with a wild type IgG1 Fc domain. In some embodiments, the mutated IgG1 Fc domain comprises the mutations L234A, L235A, P238S, H268Q, and K274Q.

In some embodiments, the anti-CD73 antibody or antigen-binding fragment comprises a heavy chain comprising an amino acid sequence selected from SEQ ID NO: 61, 62, 63, or 64 and a light chain comprising an amino acid sequence selected from SEQ ID NO: 65 or 66. In some embodiments, the anti-CD73 antibody or antigen-binding fragment comprises a heavy chain comprising an amino acid sequence selected from SEQ ID NO: 67, 68, 69, or 70 and a light chain comprising an amino acid sequence selected from SEQ ID NO: 71 or 72. In some embodiments, the anti-CD73 antibody or antigen-binding fragment comprises a heavy chain comprising an amino acid sequence selected from SEQ ID NO: 73, 74, 75, or 76 and a light chain comprising an amino acid sequence selected from SEQ ID NO: 77 or 78. In some embodiments, the anti-CD73 antibody or antigen-binding fragment comprises a heavy chain comprising an amino acid sequence selected from SEQ ID NO: 79, 80, 81, or 82 and a light chain comprising an amino acid sequence selected from SEQ ID NO: 83 or 84.

In some embodiments, the anti-CD73 antibody or antigen-binding fragment comprises a heavy chain comprising an amino acid sequence of SEQ ID NO: 63 and a light chain comprising an amino acid sequence of SEQ ID NO: 65. In some embodiments, the anti-CD73 antibody or antigen-binding fragment comprises a heavy chain comprising an amino acid sequence of SEQ ID NO: 63 and a light chain comprising an amino acid sequence of SEQ ID NO: 66. In some embodiments, the anti-CD73 antibody or antigen-binding fragment comprises a heavy chain comprising an amino acid sequence of SEQ ID NO: 64 and a light chain comprising an amino acid sequence of SEQ ID NO: 65. In some embodiments, the anti-CD73 antibody or antigen-binding fragment comprises a heavy chain comprising an amino acid sequence of SEQ ID NO: 64 and a light chain comprising an amino acid sequence of SEQ ID NO: 66. In some embodiments, the anti-CD73 antibody or antigen-binding fragment comprises a heavy chain comprising an amino acid sequence of SEQ ID NO: 69 and a light chain comprising an amino acid sequence of SEQ ID NO: 71. In some embodiments, the anti-CD73 antibody or antigen-binding fragment comprises a heavy chain comprising an amino acid sequence of SEQ ID NO: 69 and a light chain comprising an amino acid sequence of SEQ ID NO: 72. In some embodiments, the anti-CD73 antibody or antigen-binding fragment comprises a heavy chain comprising an amino acid sequence of SEQ ID NO: 70 and a light chain comprising an amino acid sequence of SEQ ID NO: 71. In some embodiments, the anti-CD73 antibody or antigen-binding fragment comprises a heavy chain comprising an amino acid sequence of SEQ ID NO: 70 and a light chain comprising an amino acid sequence of SEQ ID NO: 72.

In some embodiments, the anti-CD73 antibody or antigen-binding fragment comprises a heavy chain comprising an amino acid sequence selected from SEQ ID NO: 111, 112, 113, or 114 and a light chain comprising an amino acid sequence selected from SEQ ID NO: 65 or 66. In some embodiments, the anti-CD73 antibody or antigen-binding fragment comprises a heavy chain comprising an amino acid sequence selected from SEQ ID NO: 115, 116, 117, or 118 and a light chain comprising an amino acid sequence selected from SEQ ID NO: 71 or 72. In some embodiments, the anti-CD73 antibody or antigen-binding fragment comprises a heavy chain comprising an amino acid sequence selected from SEQ ID NO: 119, 120, 121, or 122 and a light chain comprising an amino acid sequence selected from SEQ ID NO: 77 or 78. In some embodiments, the anti-CD73 antibody or antigen-binding fragment comprises a heavy chain comprising an amino acid sequence selected from SEQ ID NO: 123, 124, 125, or 126 and a light chain comprising an amino acid sequence selected from SEQ ID NO: 83 or 84.

In some embodiments, the anti-CD73 antibody or antigen-binding fragment comprises part of a bispecific or multi-specific binding construct. In some embodiments, the anti-CD73 antibody or antigen-binding fragment is linked to a therapeutic agent or detectable agent.

In various embodiments, the present disclosure provides an antibody-drug conjugate of Formula (I):

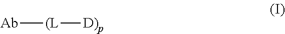

wherein Ab is an anti-CD73 antibody or antigen-binding fragment thereof disclosed herein; D is a therapeutic agent; L is a cleavable linker that covalently attaches Ab to D; and p is an integer from 1 to 20.

In some embodiments, the therapeutic agent comprises a STING agonist.

In some embodiments, the therapeutic agent comprises a compound selected from:

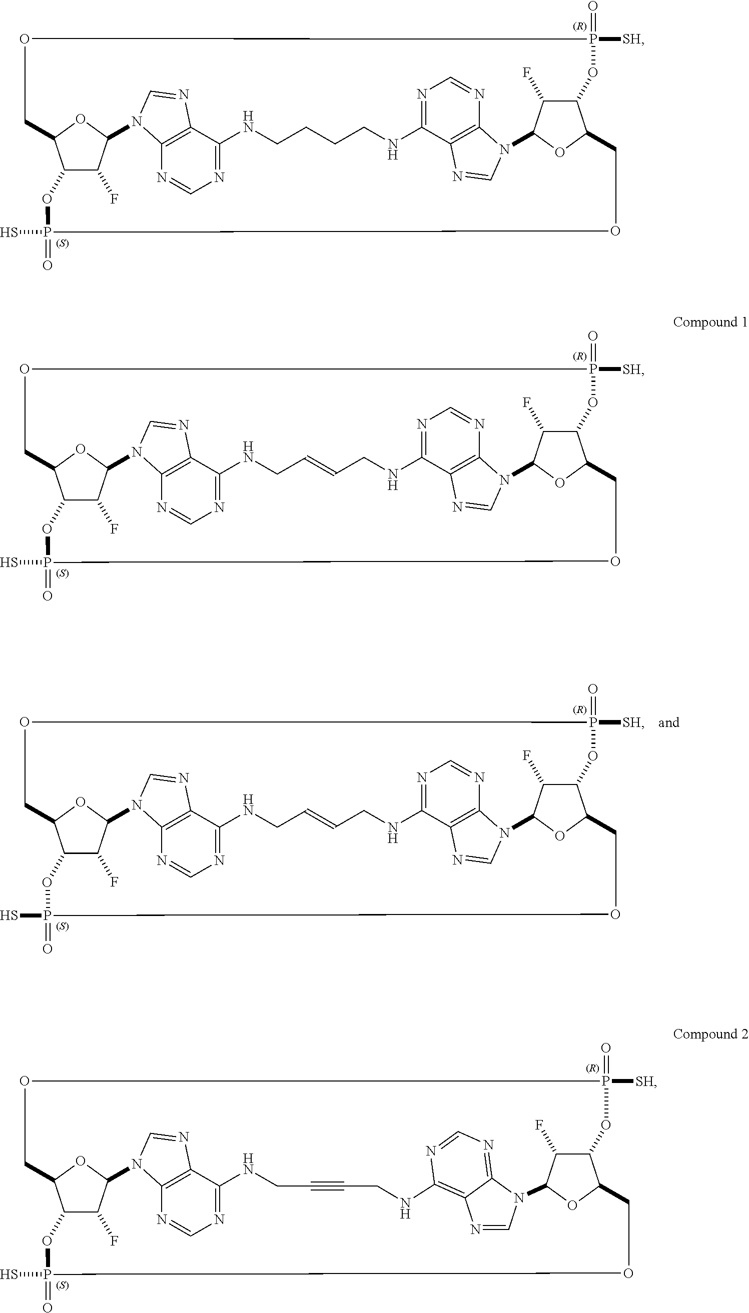

or

In some embodiments, D comprises Compound 1.

In some embodiments, D comprises Compound 2.

In some embodiments, p is from 2 to 8. In some embodiments, p is 2 or 4.

In some embodiments, the cleavable linker comprises a cleavable moiety that is positioned such that no part of the linker or the antibody or antigen-binding fragment remains bound to the cytotoxic agent upon cleavage.

In some embodiments, the cleavable linker comprises a cleavable peptide moiety. In some embodiments, the cleavable peptide moiety is cleavable by a protease. In some embodiments, the cleavable peptide moiety is cleavable by cathepsin. In some embodiments, the cleavable peptide moiety is cleavable by legumain.

In some embodiments, the cleavable peptide moiety comprises an amino acid unit. In some embodiments, the amino acid unit comprises valine-alanine (Val-Ala), valine-citrulline (Val-Cit), valine-lysine (Val-Lys), alanine-alanine-asparagine (Ala-Ala-Asn), Ala-(NMe)Ala-Asn, glycine-glycine-phenylalanine-glycine (Gly-Gly-Phe-Gly (SEQ ID NO: 244)), alanine-dimethylated lysine (Ala-Lys(Me)₂), valine-dimethylated lysine (Val-Lys(Me)₂), asparagine (Asn), aspartic acid (Asp), methylated aspartic acid (Asp(OMe)), glutamic acid-valine-alanine (Glu-Val-Ala), or glycine-valine-alanine (Gly-Val-Ala). In some embodiments, the amino acid unit comprises Val-Ala. In some embodiments, the amino acid unit comprises Val-Cit. In some embodiments, the amino acid unit comprises Gly-Gly-Phe-Gly (SEQ ID NO: 244).

In some embodiments, the cleavable linker attaches to the antibody or antigen-binding fragment via a maleimide (Mal) moiety. In some embodiments, the Mal moiety comprises a maleimidocaproyl (MC) moiety. In some embodiments, the Mal moiety comprises a dithiomaleimide (DTM) moiety. In some embodiments, the Mal moiety is reactive with a cysteine residue on the antibody or antigen-binding fragment. In some embodiments, the Mal moiety is joined to the antibody or antigen-binding fragment via a cysteine residue on the antibody or antigen-binding fragment.

In some embodiments, the cleavable linker comprises the Mal moiety and a cleavable peptide moiety. In some embodiments, the Mal moiety attaches the antibody or antigen-binding fragment to the cleavable peptide moiety in the linker. In some embodiments, the cleavable linker comprises MC-Val-Ala. In some embodiments, the cleavable linker comprises MC-Val-Cit. In some embodiments, the cleavable linker comprises MC-Gly-Gly-Phe-Gly (SEQ ID NO: 249).

In some embodiments, the cleavable linker comprises at least one spacer unit. In some embodiments, the spacer unit comprises a polyethylene glycol (PEG) moiety. In some embodiments, the PEG moiety comprises -(PEG)ₘ- and m is an integer from 1 to 10. In some embodiments, m is 2 to 8. In some embodiments, m is 2 to 5. In some embodiments, m is 2.

In some embodiments, the linker comprises at least one self-immolative unit. In some embodiments, the linker comprises a first self-immolative unit. In some embodiments, the linker is capable of being removed from D after cleavage of the linker by self-immolation of the first self-immolative unit.

In some embodiments, the first self-immolative unit comprises a p-aminobenzyl (pAB). In some embodiments, the cleavable linker comprises MC-Val-Ala-pAB. In some embodiments, the cleavable linker comprises MC-Val-Cit-pAB. In some embodiments, the cleavable linker comprises MC-Gly-Gly-Phe-Gly-pAB (SEQ ID NO: 250).

In some embodiments, the first self-immolative unit comprises a p-aminobenzyloxycarbonyl (pABC). In some embodiments, the cleavable linker comprises MC-Val-Ala-pABC. In some embodiments, the cleavable linker comprises MC-Val-Cit-pABC. In some embodiments, the cleavable linker comprises MC-Gly-Gly-Phe-Gly-pABC (SEQ ID NO: 251).

In some embodiments, the linker further comprises a second self-immolative unit. In some embodiments, the linker is capable of being removed from D after cleavage of the linker by self-immolation of the first self-immolative unit and/or self-immolation of the second self-immolative unit. In some embodiments, the linker is removed from D after cleavage of the linker in a stepwise fashion by self-immolation of the first self-immolative unit and then self-immolation of the second self-immolative unit.

In some embodiments, the linker comprises a cleavable linker, a first self-immolative unit, and a second self-immolative unit. In some embodiments, the cleavable linker comprises Val-Ala. In some embodiments, the cleavable linker comprises Val-Cit. In some embodiments, the cleavable linker comprises Gly-Gly-Phe-Gly (SEQ ID NO: 244).

In some embodiments, the second self-immolative unit comprises one of the following moieties:

| Second Self- | |
|---|---|
| immolative Unit | Chemical Structure |
| Unit 1 (MEC) | 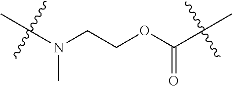 [SMILES: *C(=O)OCCN(*)C] |
| Unit 2 | 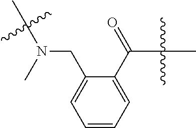 [SMILES: *C(=O)c1ccccc1CN(*)C] |
| Unit 3 | 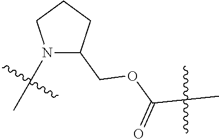 [SMILES: *C(=O)OCC1CCCN1*] |

or an isomer thereof.

In some embodiments, the cleavable linker comprises Val-Ala and the second self-immolative unit comprises one of the following moieties:

| Second Self- | |
|---|---|
| immolative Unit | Chemical Structure |
| Unit 1 (MEC) | 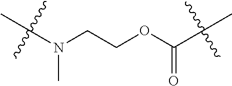 [SMILES: *C(=O)OCCN(*)C] |
| Unit 2 | 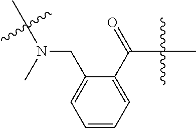 [SMILES: *C(=O)c1ccccc1CN(*)C] |
| Unit 3 | 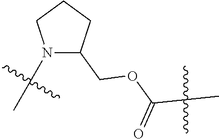 [SMILES: *C(=O)OCC1CCCN1*] |

or an isomer thereof.

In some embodiments, the second self-immolative unit comprises a Unit 1 (MEC) moiety. In some embodiments, the second self-immolative unit comprises a Unit 2 moiety. In some embodiments, the second self-immolative unit comprises a Unit 3 moiety.

In some embodiments, the linker comprises Val-Ala-pABC-Unit 1 moiety. In some embodiments, the linker comprises MC-Val-Ala-pABC-Unit 1 moiety.

In some embodiments, the L-D comprises LP2:

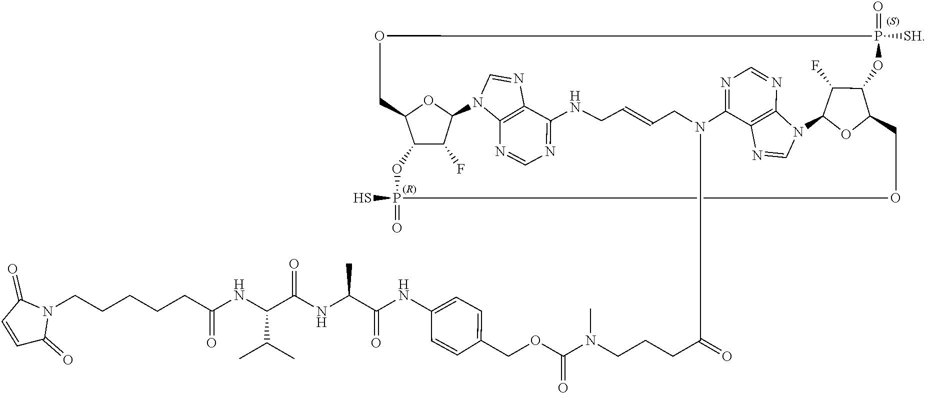

In some embodiments, the linker comprises Val-Cit-pABC-Unit 1 moiety. In some embodiments, the linker comprises MC-Val-Cit-pABC-Unit 1 moiety. In some embodiments, the L-D comprises MC-Val-Cit-pABC-Unit 1-Compound 1.

In some embodiments, the linker comprises Val-Ala-pABC-Unit 2 moiety. In some embodiments, the linker comprises MC-Val-Ala-pABC-Unit 2 moiety.

In some embodiments, the L-D comprises LP16:

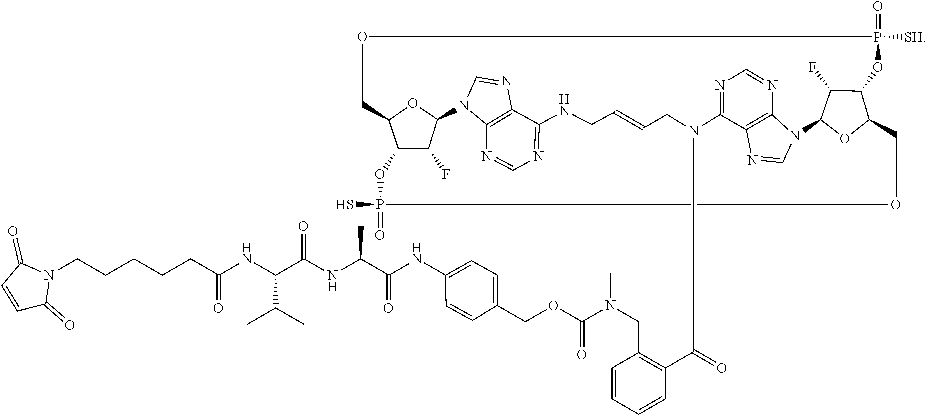 [SMILES: CC(C)[C@H](NC(=O)CCCCCN1C(=O)C=CC1=O)C(=O)N[C@@H](C)C(=O)Nc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

In some embodiments, the linker comprises Val-Cit-pABC-Unit 2 moiety. In some embodiments, the linker comprises MC-Val-Cit-pABC-Unit 2 moiety. In some embodiments, the L-D comprises MC-Val-Cit-pABC-Unit 2-Compound 1.

In some embodiments, the linker comprises Val-Ala-pABC-Unit 3 moiety. In some embodiments, the linker comprises MC-Val-Ala-pABC-Unit 3 moiety.

In some embodiments, the L-D comprises LP20:

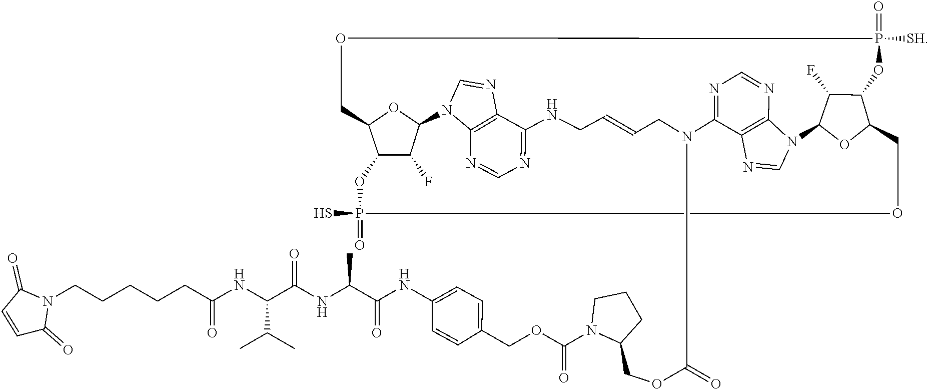 [SMILES: CC(C)[C@H](NC(=O)CCCCCN1C(=O)C=CC1=O)C(=O)N[C@@H](C)C(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

In some embodiments, the linker comprises Val-Cit-pABC-Unit 3 moiety. In some embodiments, the linker comprises MC-Val-Cit-pABC-Unit 3 moiety. In some embodiments, the L-D comprises MC-Val-Cit-pABC-Unit 3-Compound 1.

In some embodiments, the linker comprises Mal-Formula (II)-Val-Ala-pABC-Unit 2 moiety.

In some embodiments, the L-D comprises LP54:

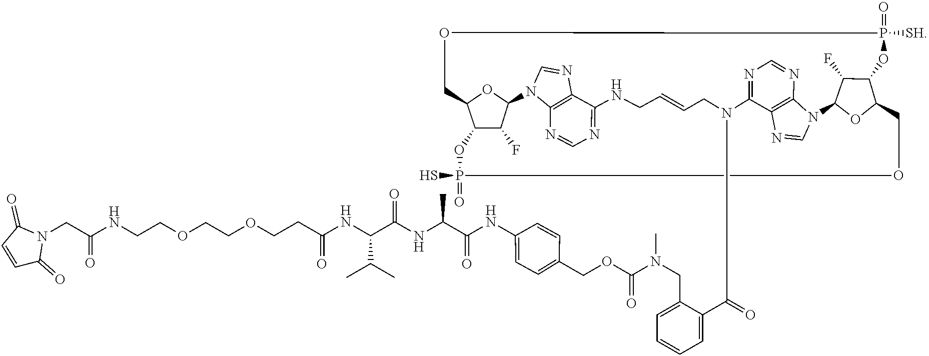 [SMILES: CC(C)[C@H](NC(=O)CCOCCOCCNC(=O)CN1C(=O)C=CC1=O)C(=O)N[C@@H](C)C(=O)Nc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

In some embodiments, the linker comprises Mal-Formula (II)-Val-Cit-pABC-Unit 2 moiety.

In some embodiments, the linker comprises Mal-(PEG)₂-Val-Ala-pABC-Unit 2 moiety.

In some embodiments, the L-D comprises LP55:

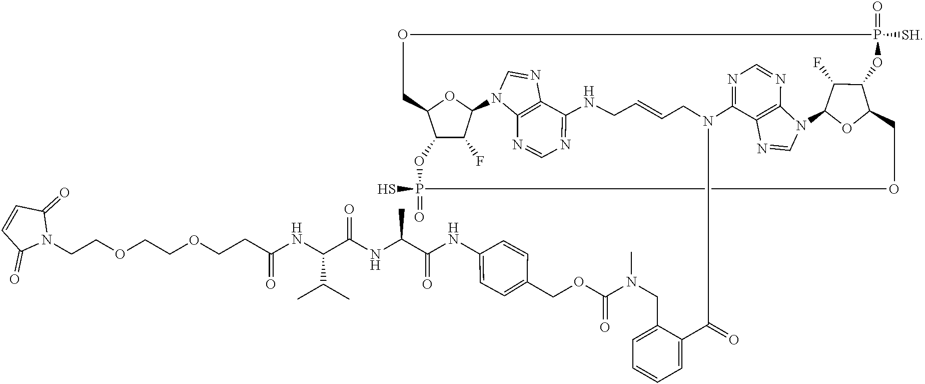 [SMILES: CC(C)[C@H](NC(=O)CCOCCOCCN1C(=O)C=CC1=O)C(=O)N[C@@H](C)C(=O)Nc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

In some embodiments, the linker comprises Mal-(PEG)₂-Val-Cit-pABC-Unit 2 moiety.

In some embodiments, the antibody or antigen-binding fragment comprises three HCDRs comprising amino acid sequences of SEQ ID NO: 1 (HCDR1), SEQ ID NO: 2 (HCDR2), and SEQ ID NO: 3 (HCDR3); and three LCDRs comprising amino acid sequences of SEQ ID NO: 4 (LCDR1), SEQ ID NO: 5 (LCDR2), and SEQ ID NO: 6 (LCDR3), as defined by the Kabat numbering system.

In some embodiments, the antibody or antigen-binding fragment comprises three HCDRs comprising amino acid sequences of SEQ ID NO: 7 (HCDR1), SEQ ID NO: 8 (HCDR2), and SEQ ID NO: 9 (HCDR3); and three LCDRs comprising amino acid sequences of SEQ ID NO: 10 (LCDR1), SEQ ID NO: 11 (LCDR2), and SEQ ID NO: 12 (LCDR3), as defined by the IGMT numbering system.

In some embodiments, the antibody or antigen-binding fragment comprises a heavy chain variable region comprising an amino acid sequence of SEQ ID NO: 49, and a light chain variable region comprising an amino acid sequence of SEQ ID NO: 50.

In some embodiments, the antibody or antigen-binding fragment comprises a heavy chain variable region comprising an amino acid sequence of SEQ ID NO: 49, and a light chain variable region comprising an amino acid sequence of SEQ ID NO: 51.

In some embodiments, the antibody or antigen-binding fragment comprises a heavy chain comprising an amino acid sequence of SEQ ID NO: 63 and a light chain comprising an amino acid sequence of SEQ ID NO: 65.

In some embodiments, the antibody or antigen-binding fragment comprises a heavy chain comprising an amino acid sequence of SEQ ID NO: 64 and a light chain comprising an amino acid sequence of SEQ ID NO: 65.

In some embodiments, the antibody or antigen-binding fragment comprises a heavy chain comprising an amino acid sequence of SEQ ID NO: 63 and a light chain comprising an amino acid sequence of SEQ ID NO: 66.

In some embodiments, the antibody or antigen-binding fragment comprises a heavy chain comprising an amino acid sequence of SEQ ID NO: 64 and a light chain comprising an amino acid sequence of SEQ ID NO: 66.

In some embodiments, the antibody or antigen-binding fragment comprises three HCDRs comprising amino acid sequences of SEQ ID NO: 13 (HCDR1), SEQ ID NO: 14 (HCDR2), and SEQ ID NO: 15 (HCDR3); and three LCDRs comprising amino acid sequences of SEQ ID NO: 16 (LCDR1), SEQ ID NO: 17 (LCDR2), and SEQ ID NO: 18 (LCDR3), as defined by the Kabat numbering system.

In some embodiments, the antibody or antigen-binding fragment comprises three HCDRs comprising amino acid sequences of SEQ ID NO: 19 (HCDR1), SEQ ID NO: 20 (HCDR2), and SEQ ID NO: 21 (HCDR3); and three LCDRs comprising amino acid sequences of SEQ ID NO: 22 (LCDR1), SEQ ID NO: 23 (LCDR2), and SEQ ID NO: 24 (LCDR3), as defined by the IGMT numbering system.

In some embodiments, the antibody or antigen-binding fragment comprises a heavy chain variable region comprising an amino acid sequence of SEQ ID NO: 52, and a light chain variable region comprising an amino acid sequence of SEQ ID NO: 53.

In some embodiments, the antibody or antigen-binding fragment comprises a heavy chain variable region comprising an amino acid sequence of SEQ ID NO: 52, and a light chain variable region comprising an amino acid sequence of SEQ ID NO: 54.

In some embodiments, the antibody or antigen-binding fragment comprises a heavy chain comprising an amino acid sequence of SEQ ID NO: 69 and a light chain comprising an amino acid sequence of SEQ ID NO: 71.

In some embodiments, the antibody or antigen-binding fragment comprises a heavy chain comprising an amino acid sequence of SEQ ID NO: 70 and a light chain comprising an amino acid sequence of SEQ ID NO: 71.

In some embodiments, the antibody or antigen-binding fragment comprises a heavy chain comprising an amino acid sequence of SEQ ID NO: 69 and a light chain comprising an amino acid sequence of SEQ ID NO: 72.

In some embodiments, the antibody or antigen-binding fragment comprises a heavy chain comprising an amino acid sequence of SEQ ID NO: 70 and a light chain comprising an amino acid sequence of SEQ ID NO: 72.

In some embodiments, the present disclosure provides an antibody-drug conjugate of Formula (I):

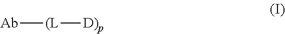

wherein Ab is an internalizing anti-CD73 antibody or internalizing antigen-binding fragment thereof comprising three HCDRs comprising amino acid sequences of SEQ ID NO: 1 (HCDR1), SEQ ID NO: 2 (HCDR2), and SEQ ID NO: 3 (HCDR3); and three LCDRs comprising amino acid sequences of SEQ ID NO: 4 (LCDR1), SEQ ID NO: 5 (LCDR2), and SEQ ID NO: 6 (LCDR3), as defined by the Kabat numbering system; or three HCDRs comprising amino acid sequences of SEQ ID NO: 7 (HCDR1), SEQ ID NO: 8 (HCDR2), and SEQ ID NO: 9 (HCDR3); and three LCDRs comprising amino acid sequences of SEQ ID NO: 10 (LCDR1), SEQ ID NO: 11 (LCDR2), and SEQ ID NO: 12 (LCDR3), as defined by the IGMT numbering system;
p is an integer from 1 to 8; and
L-D comprises LP2:

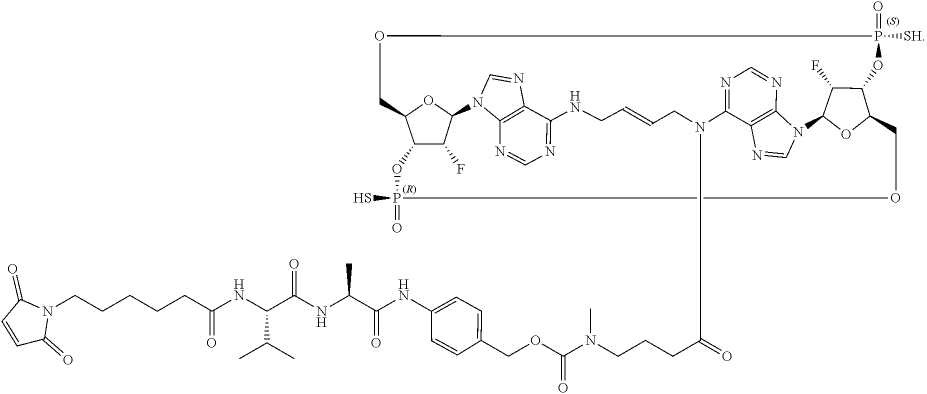

In some embodiments, the present disclosure provides an antibody-drug conjugate of Formula (I):

Ab-(L-D)ₚ (I)
wherein Ab is an internalizing anti-CD73 antibody or internalizing antigen-binding fragment thereof comprising three HCDRs comprising amino acid sequences of SEQ ID NO: 1 (HCDR1), SEQ ID NO: 2 (HCDR2), and SEQ ID NO: 3 (HCDR3); and three LCDRs comprising amino acid sequences of SEQ ID NO: 4 (LCDR1), SEQ ID NO: 5 (LCDR2), and SEQ ID NO: 6 (LCDR3), as defined by the Kabat numbering system; or three HCDRs comprising amino acid sequences of SEQ ID NO: 7 (HCDR1), SEQ ID NO: 8 (HCDR2), and SEQ ID NO: 9 (HCDR3); and three LCDRs comprising amino acid sequences of SEQ ID NO: 10 (LCDR1), SEQ ID NO: 11 (LCDR2), and SEQ ID NO: 12 (LCDR3), as defined by the IGMT numbering system;
p is an integer from 1 to 8; and
L-D comprises LP16:

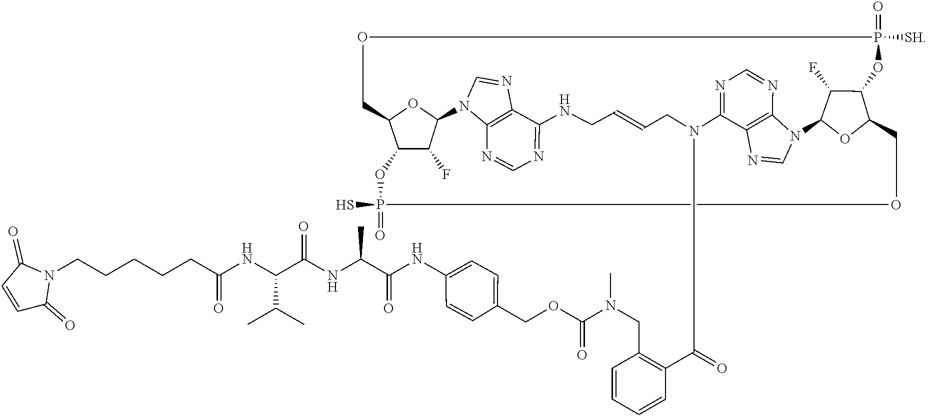 [SMILES: CC(C)[C@H](NC(=O)CCCCCN1C(=O)C=CC1=O)C(=O)N[C@@H](C)C(=O)Nc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

In some embodiments, the present disclosure provides an antibody-drug conjugate of Formula (I):

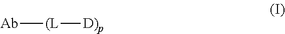

wherein Ab is an internalizing anti-CD73 antibody or internalizing antigen-binding fragment thereof comprising three HCDRs comprising amino acid sequences of SEQ ID NO: 1 (HCDR1), SEQ ID NO: 2 (HCDR2), and SEQ ID NO: 3 (HCDR3); and three LCDRs comprising amino acid sequences of SEQ ID NO: 4 (LCDR1), SEQ ID NO: 5 (LCDR2), and SEQ ID NO: 6 (LCDR3), as defined by the Kabat numbering system; or three HCDRs comprising amino acid sequences of SEQ ID NO: 7 (HCDR1), SEQ ID NO: 8 (HCDR2), and SEQ ID NO: 9 (HCDR3); and three LCDRs comprising amino acid sequences of SEQ ID NO: 10 (LCDR1), SEQ ID NO: 11 (LCDR2), and SEQ ID NO: 12 (LCDR3), as defined by the IGMT numbering system;
p is an integer from 1 to 8; and
L-D comprises LP20:

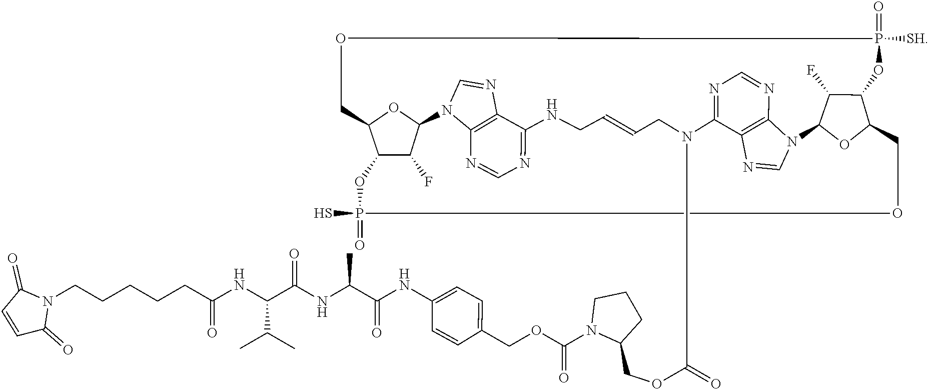 [SMILES: CC(C)[C@H](NC(=O)CCCCCN1C(=O)C=CC1=O)C(=O)N[C@@H](C)C(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

In some embodiments, the present disclosure provides an antibody-drug conjugate of Formula (I):

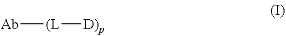

wherein Ab is an internalizing anti-CD73 antibody or internalizing antigen-binding fragment thereof comprising three HCDRs comprising amino acid sequences of SEQ ID NO: 1 (HCDR1), SEQ ID NO: 2 (HCDR2), and SEQ ID NO: 3 (HCDR3); and three LCDRs comprising amino acid sequences of SEQ ID NO: 4 (LCDR1), SEQ ID NO: 5 (LCDR2), and SEQ ID NO: 6 (LCDR3), as defined by the Kabat numbering system; or three HCDRs comprising amino acid sequences of SEQ ID NO: 7 (HCDR1), SEQ ID NO: 8 (HCDR2), and SEQ ID NO: 9 (HCDR3); and three LCDRs comprising amino acid sequences of SEQ ID NO: 10 (LCDR1), SEQ ID NO: 11 (LCDR2), and SEQ ID NO: 12 (LCDR3), as defined by the IGMT numbering system;
p is an integer from 1 to 8; and
L-D comprises LP54:

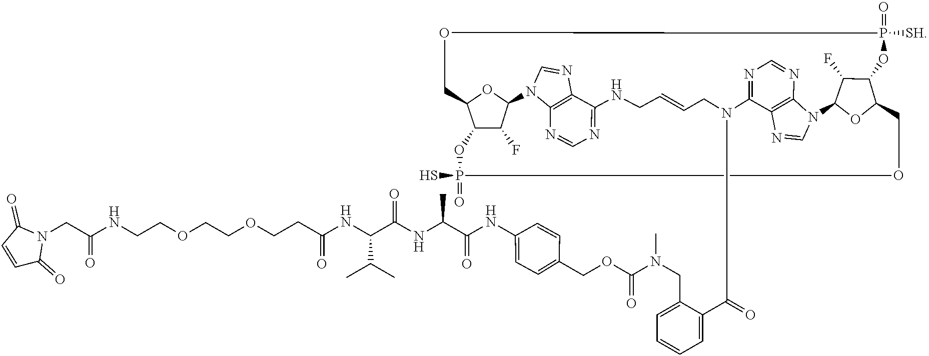 [SMILES: CC(C)[C@H](NC(=O)CCOCCOCCNC(=O)CN1C(=O)C=CC1=O)C(=O)N[C@@H](C)C(=O)Nc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

In some embodiments, the present disclosure provides an antibody-drug conjugate of Formula (I):

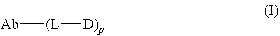

wherein Ab is an internalizing anti-CD73 antibody or internalizing antigen-binding fragment thereof comprising three HCDRs comprising amino acid sequences of SEQ ID NO: 1 (HCDR1), SEQ ID NO: 2 (HCDR2), and SEQ ID NO: 3 (HCDR3); and three LCDRs comprising amino acid sequences of SEQ ID NO: 4 (LCDR1), SEQ ID NO: 5 (LCDR2), and SEQ ID NO: 6 (LCDR3), as defined by the Kabat numbering system; or three HCDRs comprising amino acid sequences of SEQ ID NO: 7 (HCDR1), SEQ ID NO: 8 (HCDR2), and SEQ ID NO: 9 (HCDR3); and three LCDRs comprising amino acid sequences of SEQ ID NO: 10 (LCDR1), SEQ ID NO: 11 (LCDR2), and SEQ ID NO: 12 (LCDR3), as defined by the IGMT numbering system;
p is an integer from 1 to 8; and
L-D comprises LP55:

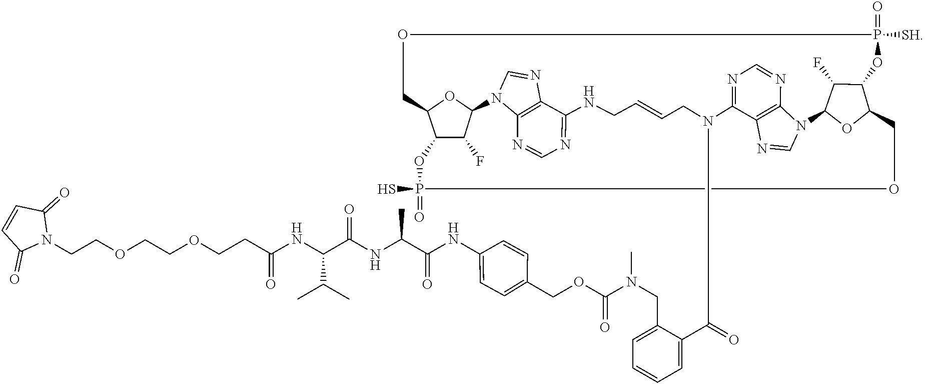 [SMILES: CC(C)[C@H](NC(=O)CCOCCOCCN1C(=O)C=CC1=O)C(=O)N[C@@H](C)C(=O)Nc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

In some embodiments, the antibody or antigen-binding fragment comprises a heavy chain variable region comprising an amino acid sequence of SEQ ID NO: 49, and a light chain variable region comprising an amino acid sequence of SEQ ID NO: 50. In some embodiments, the antibody or antigen-binding fragment comprises a heavy chain variable region comprising an amino acid sequence of SEQ ID NO: 49, and a light chain variable region comprising an amino acid sequence of SEQ ID NO: 51.

In some embodiments, the present disclosure provides an antibody-drug conjugate of Formula (I):

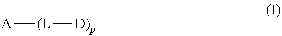

wherein Ab is an internalizing anti-CD73 antibody or internalizing antigen-binding fragment thereof comprising three HCDRs comprising amino acid sequences of SEQ ID NO: 13 (HCDR1), SEQ ID NO: 14 (HCDR2), and SEQ ID NO: 15 (HCDR3); and three LCDRs comprising amino acid sequences of SEQ ID NO: 16 (LCDR1), SEQ ID NO: 17 (LCDR2), and SEQ ID NO: 18 (LCDR3), as defined by the Kabat numbering system; or three HCDRs comprising amino acid sequences of SEQ ID NO: 19 (HCDR1), SEQ ID NO: 20 (HCDR2), and SEQ ID NO: 21 (HCDR3); and three LCDRs comprising amino acid sequences of SEQ ID NO: 22 (LCDR1), SEQ ID NO: 23 (LCDR2), and SEQ ID NO: 24 (LCDR3), as defined by the IGMT numbering system;
p is an integer from 1 to 8; and
L-D comprises LP2:

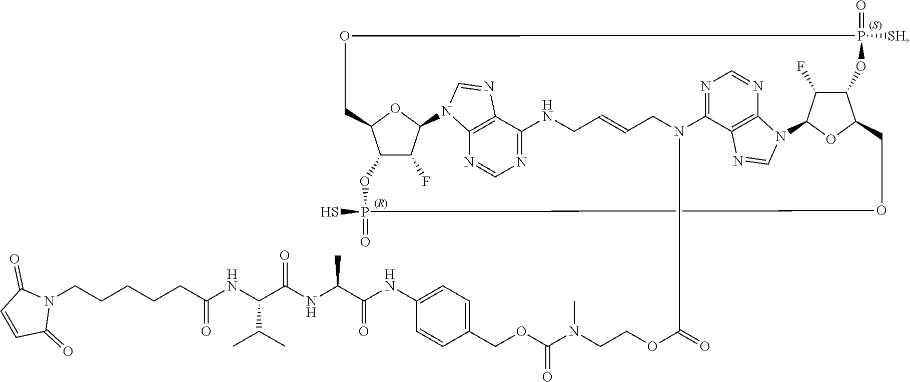

In some embodiments, the present disclosure provides an antibody-drug conjugate of Formula (I):

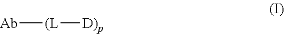

wherein Ab is an internalizing anti-CD73 antibody or internalizing antigen-binding fragment thereof comprising three HCDRs comprising amino acid sequences of SEQ ID NO: 13 (HCDR1), SEQ ID NO: 14 (HCDR2), and SEQ ID NO: 15 (HCDR3); and three LCDRs comprising amino acid sequences of SEQ ID NO: 16 (LCDR1), SEQ ID NO: 17 (LCDR2), and SEQ ID NO: 18 (LCDR3), as defined by the Kabat numbering system; or three HCDRs comprising amino acid sequences of SEQ ID NO: 19 (HCDR1), SEQ ID NO: 20 (HCDR2), and SEQ ID NO: 21 (HCDR3); and three LCDRs comprising amino acid sequences of SEQ ID NO: 22 (LCDR1), SEQ ID NO: 23 (LCDR2), and SEQ ID NO: 24 (LCDR3), as defined by the IGMT numbering system;
p is an integer from 1 to 8; and
L-D comprises LP16:

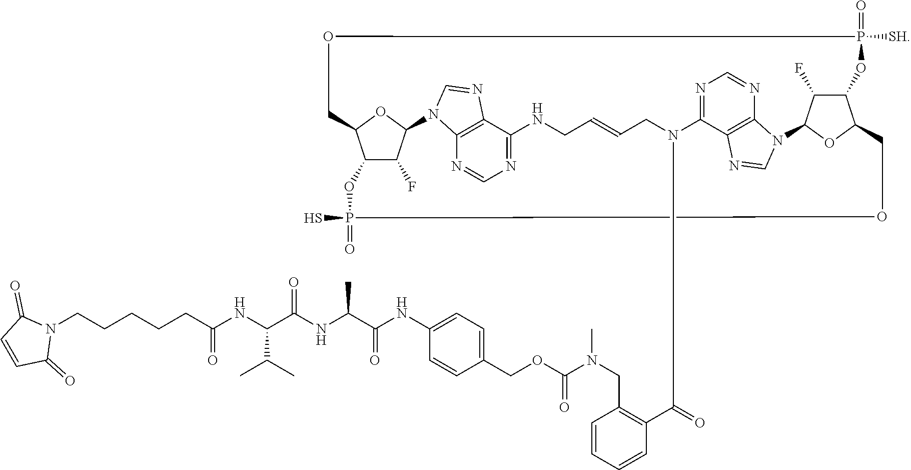 [SMILES: CC(C)[C@H](NC(=O)CCCCCN1C(=O)C=CC1=O)C(=O)N[C@@H](C)C(=O)Nc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

In some embodiments, the present disclosure provides an antibody-drug conjugate of Formula (I):

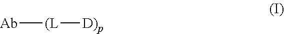

wherein Ab is an internalizing anti-CD73 antibody or internalizing antigen-binding fragment thereof comprising three HCDRs comprising amino acid sequences of SEQ ID NO: 13 (HCDR1), SEQ ID NO: 14 (HCDR2), and SEQ ID NO: 15 (HCDR3); and three LCDRs comprising amino acid sequences of SEQ ID NO: 16 (LCDR1), SEQ ID NO: 17 (LCDR2), and SEQ ID NO: 18 (LCDR3), as defined by the Kabat numbering system; or three HCDRs comprising amino acid sequences of SEQ ID NO: 19 (HCDR1), SEQ ID NO: 20 (HCDR2), and SEQ ID NO: 21 (HCDR3); and three LCDRs comprising amino acid sequences of SEQ ID NO: 22 (LCDR1), SEQ ID NO: 23 (LCDR2), and SEQ ID NO: 24 (LCDR3), as defined by the IGMT numbering system;
p is an integer from 1 to 8; and
L-D comprises LP20:

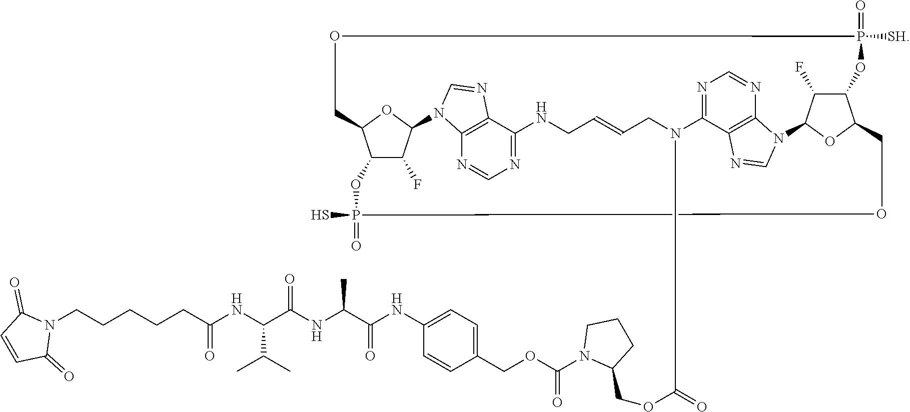 [SMILES: CC(C)[C@H](NC(=O)CCCCCN1C(=O)C=CC1=O)C(=O)N[C@@H](C)C(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

In some embodiments, the present disclosure provides an antibody-drug conjugate of Formula (I):

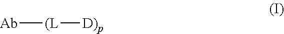

wherein Ab is an internalizing anti-CD73 antibody or internalizing antigen-binding fragment thereof comprising three HCDRs comprising amino acid sequences of SEQ ID NO: 13 (HCDR1), SEQ ID NO: 14 (HCDR2), and SEQ ID NO: 15 (HCDR3); and three LCDRs comprising amino acid sequences of SEQ ID NO: 16 (LCDR1), SEQ ID NO: 17 (LCDR2), and SEQ ID NO: 18 (LCDR3), as defined by the Kabat numbering system; or three HCDRs comprising amino acid sequences of SEQ ID NO: 19 (HCDR1), SEQ ID NO: 20 (HCDR2), and SEQ ID NO: 21 (HCDR3); and three LCDRs comprising amino acid sequences of SEQ ID NO: 22 (LCDR1), SEQ ID NO: 23 (LCDR2), and SEQ ID NO: 24 (LCDR3), as defined by the IGMT numbering system;
p is an integer from 1 to 8; and
L-D comprises LP54:

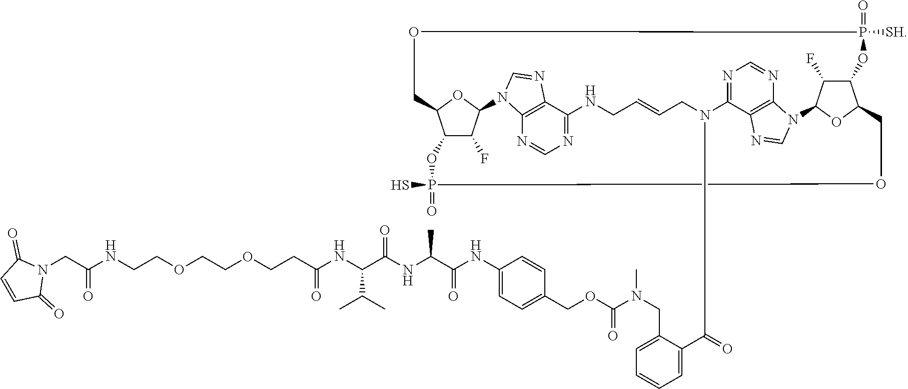 [SMILES: CC(C)[C@H](NC(=O)CCOCCOCCNC(=O)CN1C(=O)C=CC1=O)C(=O)N[C@@H](C)C(=O)Nc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

In some embodiments, the present disclosure provides an antibody-drug conjugate of Formula (I):

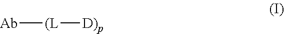

wherein Ab is an internalizing anti-CD73 antibody or internalizing antigen-binding fragment thereof comprising three HCDRs comprising amino acid sequences of SEQ ID NO: 13 (HCDR1), SEQ ID NO: 14 (HCDR2), and SEQ ID NO: 15 (HCDR3); and three LCDRs comprising amino acid sequences of SEQ ID NO: 16 (LCDR1), SEQ ID NO: 17 (LCDR2), and SEQ ID NO: 18 (LCDR3), as defined by the Kabat numbering system; or three HCDRs comprising amino acid sequences of SEQ ID NO: 19 (HCDR1), SEQ ID NO: 20 (HCDR2), and SEQ ID NO: 21 (HCDR3); and three LCDRs comprising amino acid sequences of SEQ ID NO: 22 (LCDR1), SEQ ID NO: 23 (LCDR2), and SEQ ID NO: 24 (LCDR3), as defined by the IGMT numbering system;
p is an integer from 1 to 8; and
L-D comprises LP55:

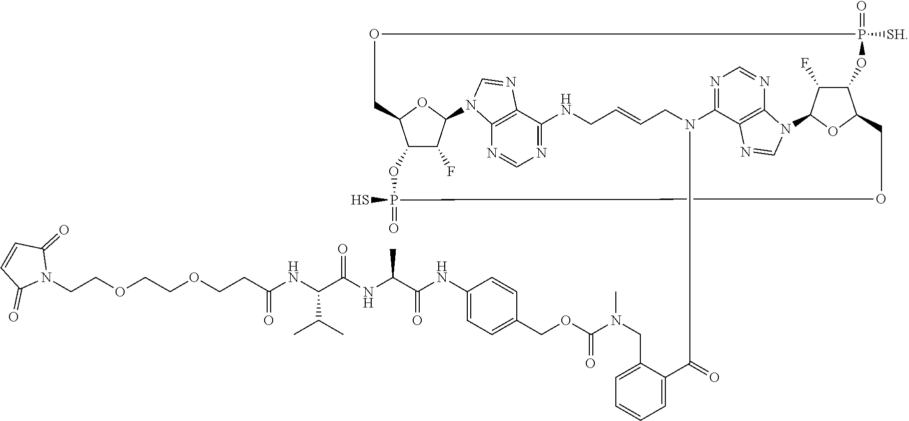 [SMILES: CC(C)[C@H](NC(=O)CCOCCOCCN1C(=O)C=CC1=O)C(=O)N[C@@H](C)C(=O)Nc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

In some embodiments, the antibody-drug conjugate comprises a heavy chain variable region comprising an amino acid sequence of SEQ ID NO: 52, and a light chain variable region comprising an amino acid sequence of SEQ ID NO: 53. In some embodiments, the antibody-drug conjugate comprises a heavy chain variable region comprising an amino acid sequence of SEQ ID NO: 52, and a light chain variable region comprising an amino acid sequence of SEQ ID NO: 54.

In some embodiments, p is 2 or 4.

In some embodiments, p is determined by hydrophobic interaction chromatography-high performance liquid chromatography (HIC-HPLC). In some embodiments, p is determined by reverse-phase liquid chromatography-mass spectrometry (LC-MS).

In various embodiments, the present disclosure provides a composition comprising multiple copies of an antibody-drug conjugate disclosed herein. In some embodiments, the average p of the antibody-drug conjugates in the composition is about 1 to about 4. In some embodiments, the average p is about 2 or about 4.

In various embodiments, the present disclosure provides a pharmaceutical composition comprising an antibody-drug conjugate disclosed herein and a pharmaceutically acceptable carrier.

In various embodiments, the present disclosure provides a method of treating a patient having or at risk of having a cancer that expresses CD73, comprising administering to the patient a therapeutically effective amount of an antibody or antigen-binding fragment disclosed herein. In various embodiments, the present disclosure provides a method of treating a patient having or at risk of having a cancer that expresses CD73, comprising administering to the patient a therapeutically effective amount of an antibody-drug conjugate disclosed herein. In various embodiments, the present disclosure provides a method of treating a patient having or at risk of having a cancer that expresses CD73, comprising administering to the patient a therapeutically effective amount of a composition disclosed herein. In various embodiments, the present disclosure provides a method of treating a patient having or at risk of having a cancer that expresses CD73, comprising administering to the patient a therapeutically effective amount of a pharmaceutical composition disclosed herein.

In various embodiments, the present disclosure provides a method of stimulating an anti-tumor immune response in the tumor microenvironment of a patient having a cancer that expresses CD73, comprising administering to the patient a therapeutically effective amount of an antibody or antigen-binding fragment disclosed herein. In various embodiments, the present disclosure provides a method of stimulating an anti-tumor immune response in the tumor microenvironment of a patient having a cancer that expresses CD73, comprising administering to the patient a therapeutically effective amount of an antibody-drug conjugate disclosed herein. In various embodiments, the present disclosure provides a method of stimulating an anti-tumor immune response in the tumor microenvironment of a patient having a cancer that expresses CD73, comprising administering to the patient a therapeutically effective amount of a composition disclosed herein. In various embodiments, the present disclosure provides a method of stimulating an anti-tumor immune response in the tumor microenvironment of a patient having a cancer that expresses CD73, comprising administering to the patient a therapeutically effective amount of a pharmaceutical composition disclosed herein.

In various embodiments, the present disclosure provides a method of reducing or inhibiting growth of a CD73-expressing tumor, comprising administering a therapeutically effective amount of an antibody or antigen-binding fragment disclosed herein. In various embodiments, the present disclosure provides a method of reducing or inhibiting growth of a CD73-expressing tumor, comprising administering a therapeutically effective amount of an antibody-drug conjugate disclosed herein. In various embodiments, the present disclosure provides a method of reducing or inhibiting growth of a CD73-expressing tumor, comprising administering a therapeutically effective amount of a composition disclosed herein. In various embodiments, the present disclosure provides a method of reducing or inhibiting growth of a CD73-expressing tumor, comprising administering a therapeutically effective amount of a pharmaceutical composition disclosed herein.

In some embodiments, the CD73-expressing cancer is melanoma, diffuse large B-cell lymphoma, T-cell lymphoma, breast cancer, ovarian cancer, head and neck cancer, head and neck squamous carcinoma, non-small-cell lung cancer (NSCLC), glioblastoma, thyroid carcinoma, pancreatic cancer, pancreatic ductal adenocarcinoma (PDAC), esophageal cancer, cervical cancer, gastric cancer, gallbladder cancer, or colorectal cancer. In some embodiments, the CD73-expressing cancer is a breast cancer, ovarian cancer, PDAC, or NSCLC.

In various embodiments, the present disclosure provides one or more nucleic acid(s) encoding an antibody or antigen-binding fragment disclosed herein. In some embodiments, the present disclosure provides a host cell comprising the one or more nucleic acids. In some embodiments, the present disclosure provides a method of producing an antibody or antigen-biding fragment disclosed herein, comprising culturing the host cell under conditions sufficient to produce the antibody or antigen-binding fragment.

In various embodiments, the present disclosure provides a method of producing an antibody-drug conjugate disclosed herein, comprising reacting an antibody or antigen-binding fragment disclosed herein with a cleavable linker joined to D under conditions that allow conjugation, wherein D comprises a compound selected from:

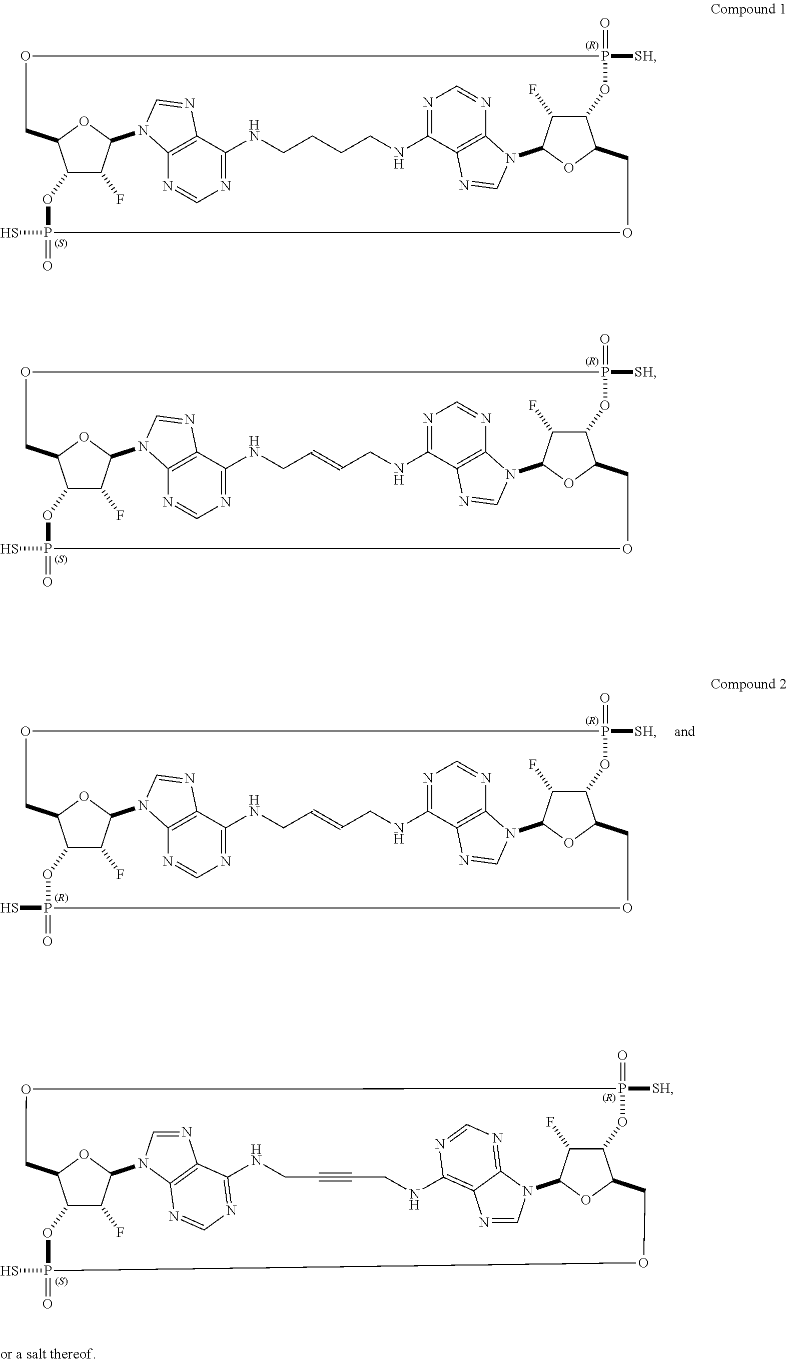

In various embodiments, the present disclosure provides a method of producing a composition disclosed herein, comprising reacting an antibody or antigen-binding fragment disclosed herein with a cleavable linker joined to D under conditions that allow conjugation, wherein D comprises a compound selected from:

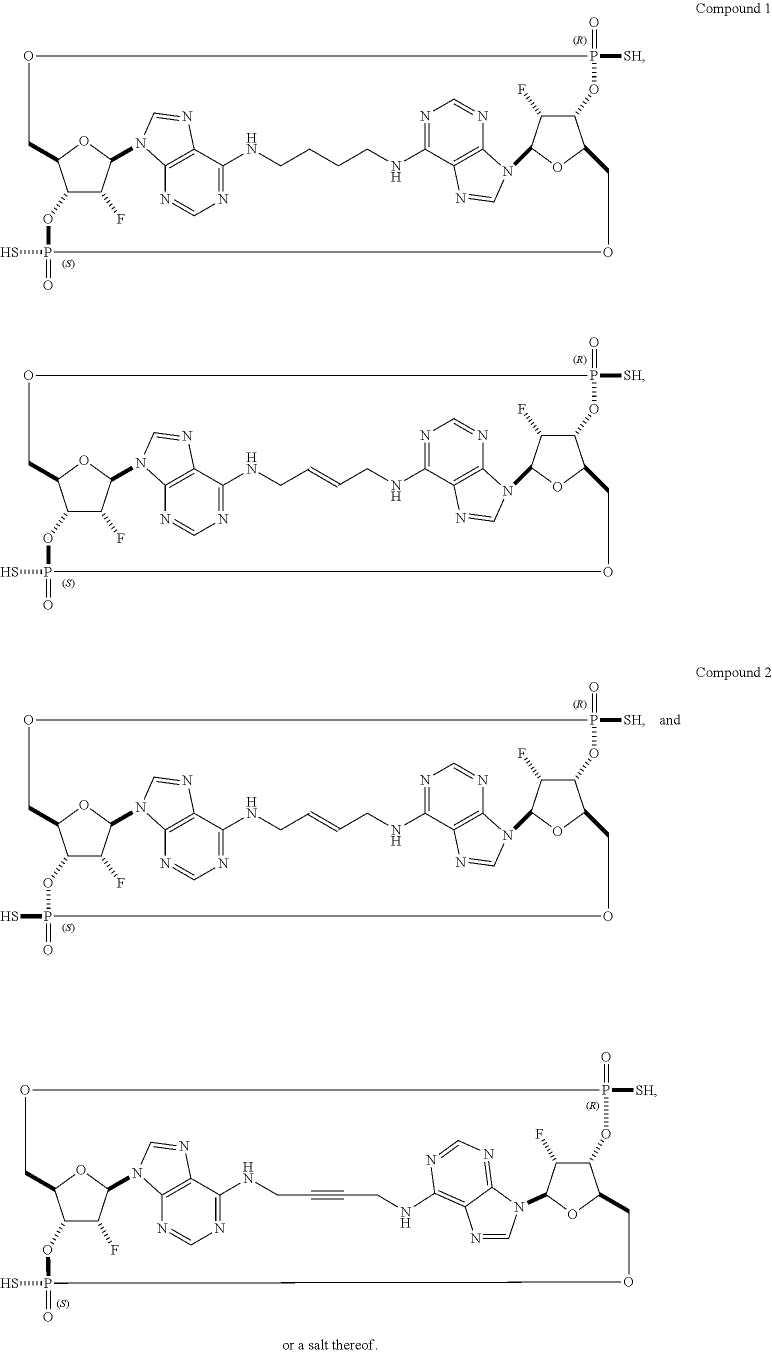

### BRIEF DESCRIPTION OF THE DRAWINGS

FIG. 1A shows binding of recombinantly-expressed antibodies to human CD73-expressing MDA-MB-231 cells as determined by FACS titration. FIG. 1B shows binding of recombinantly-expressed antibodies to mouse CD73-expressing 4T1 cells as determined by FACS titration. GRN MFI=green mean fluorescence intensity.

FIG. 2 shows enzymatic inhibition of human and mouse CD73 by anti-CD73 monoclonal antibodies in MDA-MB-231 (human CD73) and 4T1 (mouse CD73) cells.

FIGS. 3A, 3B, and 3C show the percent of internalized anti-CD73 antibody after incubation with MDA-MB-231 (FIG. 3A), MDA-MB-468 (FIG. 3B), or U87-MG (FIG. 3C) cells at the indicated time points. Samples were incubated for 0 (“4° C. quenched”), 1, 2, 3, or 4 hours and quenched with an anti-AlexaFluor488 antibody on ice.

FIG. 4 shows 190K12 and 366F5 humanized sequences. The FRs and CDRs as defined by Kabat or IMGT are shown above each clone. Mouse residues are in bold, humanized residues are underlined, and unformatted residues are homologous. From top to bottom, the sequences shown are identified as SEQ ID NOs: 255-259, 49, 260-261, 50-51, 262-264, 52, 265-269, 53-54 and 270.

FIGS. 5A, 5B, and 5C show in silico immunogenicity prediction for humanized antibody clones 190K12-H1L1 (FIG. 5A), 190K12-H4L1 (FIG. 5B), and 366F5-H2L1 (FIG. 5C).

FIG. 6 shows humanized rabbit anti-human CD73 antibody sequences. The FRs and CDRs as defined by Kabat or IMGT are shown above each clone. Rabbit residues are in bold, humanized residues are underlined, and unformatted residues are homologous. From top to bottom, the sequences shown are identified as SEQ ID NOs: 271-275, 57, 56, 276-277, 58, 278-279, 60 and 59.

FIG. 7 shows cell-surface binding of human CD73 by humanized rabbit anti-human CD73 antibodies in CD73-positive cells (MDA-MB-231) or CD73-negative cells (CHO) as determined by FACS.

FIG. 8 shows enzymatic inhibition of human CD73 in MDA-MB-231 cells.

FIG. 9 shows enzymatic inhibition of human CD73 in SK-RC-52 (panel A) and NCI-H2110 cells (panel B) at various antibody concentrations.

FIGS. 10A and 10B show in silico immunogenicity prediction of humanized antibody clones 23P11-H1L1 (FIG. 10A) and 24O6-H1L1 (FIG. 10B).

FIG. 11A shows the average DAR of anti-CD73 ADCs in buffer over time as analyzed by HIC-HPLC. FIG. 11B shows percentage of monomeric ADCs in buffer over time as analyzed by SEC.

FIG. 12 shows the stability of the ADCs in human plasma. Panel A shows the percentage of free Compound 1 in human plasma over time. Panel B shows average DAR of anti-CD73 ADCs in human plasma over time.

FIG. 13A shows average tumor growth in NCI-H2110 xenograft mice upon treatment with anti-CD73-LP2 ADCs over time. FIG. 13B shows average body weight in NCI-H2110 xenograft mice upon treatment with anti-CD73-LP2 ADCs over time.

FIG. 14 shows serum cytokine levels after treatment with anti-CD73 ADCs in an NCI-H2110 xenograft mouse model.

FIG. 15A shows average tumor volume in MDA-MB-231 xenograft mice upon treatment with anti-CD73-LP2 ADCs over time. FIG. 15B shows average body weight in MDA-MB-231 xenograft mice upon treatment with anti-CD73-LP2 ADCs over time.

FIG. 16 shows serum cytokine levels after treatment with anti-CD73 ADCs in an MDA-MB-231 xenograft mouse model.

FIG. 17A shows average tumor volume in Ca-OV-3-T2 xenograft mice upon treatment with anti-CD73-LP2 ADCs over time. FIG. 17B shows average body weight in Ca-OV-3-T2 xenograft mice upon treatment with anti-CD73-LP2 ADCs over time.

FIG. 18 shows serum cytokine levels after treatment with anti-CD73 ADCs in a Ca-OV-3-T2 xenograft mouse model.

FIG. 19A shows average tumor volume in HCC1954 xenograft mice upon treatment with anti-CD73-LP2 ADCs over time. FIG. 19B shows average body weight in HCC1954 xenograft mice upon treatment with anti-CD73-LP2 ADCs over time.

FIG. 20 shows serum cytokine levels after treatment with anti-CD73 ADCs in an HCC1954 xenograft mouse model.

FIGS. 21A and 21B show average tumor volume in NCI-H2110 xenograft mice upon treatment with the indicated anti-CD73 ADCs over time. FIG. 21C shows average body weight change relative to day 0 in NCI-H2110 xenograft mice upon treatment with the indicated anti-CD73 ADCs over time.

FIG. 22 shows serum cytokine levels after treatment with anti-CD73 ADCs in an NCI-H2110 xenograft mouse model.

FIGS. 23A and 23B show average tumor volume in MDA-MB-231 xenograft mice upon treatment with the indicated anti-CD73 ADCs over time. FIG. 23C shows average body weight change relative to day 0 in MDA-MB-231 xenograft mice upon treatment with the indicated anti-CD73 ADCs over time.

FIG. 24 shows serum cytokine levels after treatment with anti-CD73 ADCs in an MDA-MB-231 xenograft mouse model.

FIGS. 25A and 25B show average tumor volume in Ca-OV-3-T2 xenograft mice upon treatment with the indicated anti-CD73 ADCs over time. FIG. 25C shows average body weight change relative to day 0 in Ca-OV-3-T2 xenograft mice upon treatment with the indicated anti-CD73 ADCs over time.

FIG. 26 shows serum cytokine levels after treatment with anti-CD73 ADCs in a Ca-OV-3-T2 xenograft mouse model.

FIGS. 27A and 27B show average tumor volume in HCC1954 xenograft mice upon treatment with the indicated anti-CD73 ADCs over time. FIG. 27C shows average body weight change relative to day 0 in HCC1954 xenograft mice upon treatment with the indicated anti-CD73 ADCs over time.

FIG. 28 shows serum cytokine levels after treatment with anti-CD73 ADCs in an HCC1954 xenograft mouse model.

FIG. 29A shows the average DAR of anti-CD73 ADCs in buffer over time as analyzed by HIC-HPLC. FIG. 29B shows percentage of monomeric ADCs in buffer over time as analyzed by SEC.

FIG. 30A shows average DAR and percent change in DAR of anti-CD73 ADCs in human plasma over time. FIG. 30B shows the percent hydrolysis of the light chain and heavy chain of the antibody over time.

FIG. 31A shows average tumor volume in NCI-H2110 xenograft mice upon treatment with the indicated anti-CD73 ADCs over time. FIG. 31B shows average body weight change relative to day 0 in NCI-H2110 xenograft mice upon treatment with the indicated anti-CD73 ADCs over time.

### DETAILED DESCRIPTION

### Definitions

Various terms relating to aspects of the description are used throughout the specification and claims. Such terms are to be given their ordinary meaning in the art unless otherwise indicated. Other specifically defined terms are to be construed in a manner consistent with the definitions provided herein.

As used herein, the singular forms “a,” “an,” and “the” include plural forms unless the context clearly dictates otherwise.

The terms “about” or “approximately” in the context of numerical values and ranges refer to values or ranges that approximate or are close to the recited values or ranges such that the embodiment may perform as intended, such as having a desired amount of nucleic acids or polypeptides in a reaction mixture, as is apparent to the skilled person from the teachings contained herein. In some embodiments, “about” means plus or minus 10% of a numerical amount.

The term “agent” is used herein to refer to a chemical compound, a mixture of chemical compounds, a biological macromolecule, or an extract made from biological materials. The term “therapeutic agent,” “drug,” or “drug moiety” refers to an agent that is capable of modulating a biological process and/or has biological activity.

The terms “antibody-drug conjugate,” “antibody conjugate,” “conjugate,” “immunoconjugate,” and “ADC” are used interchangeably, and refer to a compound or derivative thereof that is linked to an antibody or antigen-binding fragment (e.g., an anti-CD73 antibody) and may be defined by the generic formula: Ab-(L-D), (Formula I), wherein Ab comprises an antibody moiety (i.e., an antibody or antigen-binding fragment), L comprises a linker moiety, D comprises a drug moiety, and p is the number of drug moieties per antibody moiety. In some embodiments, the linker L can include a cleavable moiety between the antibody or antigen-binding fragment and the therapeutic compound. In some embodiments, the linker L can include a cleavable moiety that can be attached to either or both the antibody or antigen-binding fragment and to the therapeutic compound, e.g., by spacer unit(s). Exemplary cleavable linkers are described and exemplified herein. An antibody-drug conjugate disclosed herein may be conjugated to more than one drug, e.g., using a linker disclosed herein.

The term “antibody” is used in the broadest sense to refer to an immunoglobulin molecule that recognizes and specifically binds to a target, such as a protein, polypeptide, carbohydrate, polynucleotide, lipid, or combinations of the foregoing through at least one antigen recognition site within the variable region of the immunoglobulin molecule. The heavy chain (HC) of an antibody is composed of a heavy chain variable domain (VH) and a heavy chain constant region (CH). The light chain (LC) is composed of a light chain variable domain (VL) and a light chain constant domain (CL). As used herein, the terms “domain” and “region” may be used interchangeably (e.g., the term “variable domain” may be used interchangeably with the term “variable region” and understood to refer to the same part of the antibody). For the purposes of this application, the mature heavy chain and light chain variable domains each comprise three complementarity determining regions (CDR1, CDR2, and CDR3) (also referred to as “hypervariable regions”) within four framework regions (FR1, FR2, FR3, and FR4) arranged from N-terminus to C-terminus: FRI, CDR1, FR2, CDR2, FR3, CDR3, and FR4. CDRs may be identified according to the Kabat and/or IMGT numbering systems (Kabat, Sequences of Proteins of Immunological Interest (National Institutes of Health, Bethesda, Md. (1987 and 1991); International ImMunoGeneTics Information System (IMGT®)). An “antibody” can be naturally occurring or man-made, such as monoclonal antibodies produced by conventional hybridoma technology. The term “antibody” includes full-length monoclonal antibodies and full-length polyclonal antibodies, as well as antibody fragments such as Fab, Fab′, F(ab′)2, Fv, and single chain antibodies. An antibody can be any one of the five major classes of immunoglobulins: IgA, IgD, IgE, IgG, and IgM, or subclasses thereof (e.g., isotypes IgG1, IgG2, IgG3, IgG4). An antibody of any of the aforementioned class or subclass can also comprise one of two functionally similar classes of light chains: Igκ (also referred to herein as “Ig kappa” or “kappa”) and Igλ (also referred to herein as “Ig lambda” or “lambda”). The term antibody encompasses human antibodies, chimeric antibodies, humanized antibodies, and any modified immunoglobulin molecule containing an antigen recognition site, so long as it demonstrates the desired biological activity.

The term “chimeric antibody,” as used herein, refers to antibodies wherein the amino acid sequence of the immunoglobulin molecule is derived from two or more species. In some instances, the variable regions of both heavy and light chains correspond to the variable regions of antibodies derived from one species with the desired specificity, affinity, and activity while the constant regions are homologous to antibodies derived from another species (e.g., human) to minimize an immune response in the latter species.

The term “human antibody,” as used herein, refers to an antibody produced by a human or an antibody having an amino acid sequence of an antibody produced by a human.

As used herein, the term “humanized antibody” refers to forms of antibodies that contain sequences from non-human (e.g., mouse or rabbit) antibodies as well as human antibodies. Such antibodies are chimeric antibodies which contain minimal sequence derived from non-human immunoglobulin. In general, the humanized antibody will comprise substantially all of at least one, and typically two, variable domains, in which all or substantially all of the hypervariable regions correspond to those of a non-human immunoglobulin and all or substantially all of the framework (FR) regions are those of a human immunoglobulin sequence. The humanized antibody optionally also will comprise at least a portion of an immunoglobulin constant region (Fc), typically that of a human immunoglobulin. The humanized antibody can be further modified by the substitution of residues, either in the Fv framework region and/or within the replaced non-human residues to refine and optimize antibody specificity, affinity, and/or activity. The humanized antibody can also be further modified by the substitution of residues in the Fc domain to reduce its binding to various cell receptors, such as a Fcγ receptor (FcγR), and other immune molecules.

The term “monoclonal antibody,” as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic epitope. In contrast, conventional (polyclonal) antibody preparations typically include a multitude of antibodies directed against (or specific for) different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al. (1975) Nature 256:495 or may be made by recombinant DNA methods (see, e.g., U.S. Pat. No. 4,816,567). Monoclonal antibodies may also be isolated from phage antibody libraries using the techniques described in Clackson et al. (1991) Nature 352:624-8, and Marks et al. (1991) J. Mol. Biol. 222:581-97, for example.

The monoclonal antibodies described herein specifically include “chimeric” antibodies, in which a portion of the heavy and/or light chain is identical with or homologous to corresponding sequences in an antibody derived from a particular species or belonging to a particular antibody class or subclass, while the remainder of the chain(s) is identical with or homologous to corresponding sequences in an antibody derived from another species or belonging to another antibody class or subclass, as well as fragments of such antibodies, so long as they specifically bind the target antigen and/or exhibit the desired biological activity.

The term “antigen-binding fragment” or “antigen-binding portion” of an antibody, as used herein, refers to one or more fragments of an antibody that retain the ability to specifically bind to an antigen (e.g., CD73). Antigen-binding fragments preferably also retain the ability to internalize into an antigen-expressing cell. In some embodiments, antigen-binding fragments also retain immune effector activity. It has been shown that fragments of a full-length antibody can perform the antigen-binding function of a full-length antibody. Examples of binding fragments encompassed within the term “antigen-binding fragment” or “antigen-binding portion” of an antibody include (i) a Fab fragment, a monovalent fragment consisting of the VL, VH, CL, and Cm domains; (ii) a F(ab′)2 fragment, a bivalent fragment comprising two Fab fragments linked by a disulfide bridge at the hinge region; (iii) a Fd fragment consisting of the VH and Cm domains; (iv) a Fv fragment consisting of the VL and VH domains of a single arm of an antibody; (v) a dAb fragment, which comprises a single variable domain, e.g., a VH domain (see, e.g., Ward et al. (1989) Nature 341:544-6; and Winter et al., WO 90/05144); and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv)). See, e.g., Bird et al. (1988) Science 242:423-6; and Huston et al. (1988) Proc. Natl. Acad. Sci. USA 85:5879-83. Such single chain antibodies are also intended to be encompassed within the term “antigen-binding fragment” or “antigen-binding portion” of an antibody, and are known in the art as an exemplary type of binding fragment that can internalize into cells upon binding. See, e.g., Zhu et al. (2010) 9:2131-41; He et al. (2010) J. Nucl. Med. 51:427-32; and Fitting et al. (2015) MAbs 7:390-402. In certain embodiments, scFv molecules may be incorporated into a fusion protein. Other forms of single chain antibodies, such as diabodies, are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen-binding sites. See, e.g., Holliger et al. (1993) Proc. Natl. Acad. Sci. USA 90:6444-8; and Poljak et al. (1994) Structure 2:1121-3. Antigen-binding fragments are obtained using conventional techniques known to those of skill in the art, and the binding fragments are screened for utility (e.g., binding affinity, internalization) in the same manner as are intact antibodies. Antigen-binding fragments may be prepared, e.g., by cleavage of the intact protein, e.g., by protease or chemical cleavage.

The term “anti-CD73 antibody” or “antibody that specifically binds CD73” refers to any form of antibody or fragment thereof that specifically binds CD73, and encompasses monoclonal antibodies (including full length monoclonal antibodies), polyclonal antibodies, and biologically functional antibody fragments so long as they specifically bind CD73. Preferably the anti-CD73 antibody used in the ADCs disclosed herein is an internalizing antibody or internalizing antibody fragment. 190K12, 366F5, 23P11, and 24O6 are exemplary internalizing anti-human CD73 antibodies. As used herein, the terms “specific,” “specifically binds,” and “binds specifically” refer to the selective binding of the antibody to the target antigen or epitope over alternative antigens or epitopes. Antibodies can be tested for specificity of binding by comparing binding to an appropriate antigen to binding to an irrelevant antigen or antigen mixture under a given set of conditions. If the antibody binds to the appropriate antigen with at least 10-fold, or preferably at least 50-fold, at least 100-fold, or at least 1000-fold, more affinity than to an irrelevant antigen or antigen mixture, then it is considered to be specific, e.g., as measured by surface plasmon resonance, e.g., BIAcore® analysis. In one embodiment, a specific antibody is one that binds the CD73 antigen, but does not bind (or exhibits minimal binding) to other antigens.

The term “aryl” refers to a group or substituent derived from an aromatic ring and encompasses monocyclic aromatic rings and bicyclic, tricyclic, and fused ring systems having a total of six to fourteen ring members, wherein at least one ring in the system is aromatic. An aryl group may be optionally substituted with one or more substituents.

The term “heteroaryl” refers to a cyclic group comprising at least one ring atom that is a heteroatom, such as O, N, or S. Heteroaryl groups encompass monocyclic, bicyclic, and tricyclic ring systems having a total of five to fourteen ring members, wherein at least one ring in the system is aromatic, at least one ring in the system contains one or more heteroatoms, and wherein each ring in the system contains three to seven ring members.

The term “bridge” refers to a grouping of atoms in a macrocycle-bridged STING agonist compound of the disclosure that extends from a first nucleic acid base in the macrocycle-bridged STING agonist compound to a second nucleic acid base in the macrocycle-bridged STING agonist compound.

The term “cancer” refers to the physiological condition in mammals in which a population of cells is characterized by unregulated cell growth. Examples of cancers include, but are not limited to, carcinoma, lymphoma, blastoma, sarcoma, and leukemia. More particular examples of such cancers include squamous cell cancer, small cell lung cancer, non-small cell lung cancer, adenocarcinoma of the lung, squamous carcinoma of the lung, bile duct cancer (e.g., cholangiocarcinoma), esophageal cancer, nasopharyngeal cancer, cancer of the peritoneum, hepatocellular cancer (e.g., hepatocellular carcinoma), gastrointestinal cancer, pancreatic cancer (e.g., pancreatic ductal adenocarcinoma), glioblastoma, cervical cancer, ovarian cancer, liver cancer, bladder cancer, hepatoma, diffuse large B-cell lymphoma, T-cell lymphoma, breast cancer, osteosarcoma, skin cancer (e.g., melanoma), colon cancer, colorectal cancer, endometrial or uterine cancer, ovarian cancer, salivary gland carcinoma, kidney cancer, liver cancer, prostate cancer, vulval cancer, thyroid cancer, hepatic carcinoma, bone cancer, and various types of head and neck cancers (e.g. head and neck squamous carcinoma).

The terms “cancer cell” and “tumor cell” refer to individual cells or the total population of cells derived from a tumor, including both non-tumorigenic cells and cancer stem cells. As used herein, the term “tumor cell” will be modified by the term “non-tumorigenic” when referring solely to those tumor cells lacking the capacity to renew and differentiate to distinguish those tumor cells from cancer stem cells.

The terms “tumor” and “neoplasm” refer to any mass of tissue that results from excessive cell growth or proliferation, either benign or malignant, including precancerous lesions.

The term “co-administration” or administration “in combination with” one or more therapeutic agents includes concurrent and consecutive administration in any order.

The term “chemotherapeutic agent” or “anti-cancer agent” is used herein to refer to a chemical compound that is effective in treating cancer regardless of mechanism of action. Inhibition of metastasis or angiogenesis is frequently a property of a chemotherapeutic agent. Stimulation of an antitumor immune response may also be a property of a chemotherapeutic agent. Non-limiting examples of chemotherapeutic agents include stimulatory agents, e.g., STING agonists. In addition, chemotherapeutic agents include antibodies, biological molecules, and small molecules. A chemotherapeutic agent may be a cytotoxic or cytostatic agent.

The term “cytotoxic agent” refers to a substance that causes cell death either by interfering with a cell's expression activity and/or functioning or by stimulating a response that causes cell death, e.g., an immune response. Examples of cytotoxic agents include, but are not limited to, STING agonists such as Compound 1.

The term “cluster of differentiation 73,” or “CD73” as used herein, refers to any native form of human CD73. The term encompasses full-length CD73 (e.g., NCBI Reference Sequence: NP_002517.1; SEQ ID NO: 248), as well as any form of human CD73 that results from cellular processing. The term also encompasses naturally occurring variants of CD73. An antibody that binds CD73 may not bind all variants, as will be readily apparent to one of skill in the art. CD73 can be isolated from a human, or may be produced recombinantly or by synthetic methods. The terms “CD73” and “cluster of differentiation 73” are interchangeable with “ecto-5-prime nucleotidase,” “NT5E,” “E5NT,” “ENT,” “NTE,” “nucleotidase,” “ecto-5-prime,” “NT5,” and any other names for proteins encoded by NT5E known in the art.

An “effective amount” of an ADC as disclosed herein is an amount sufficient to perform a specifically stated purpose, for example to produce a therapeutic effect after administration, such as a reduction in tumor growth rate or tumor volume, a reduction in a symptom of cancer, or some other indicia of treatment efficacy. An effective amount can be determined in a routine manner in relation to the stated purpose. The term “therapeutically effective amount” refers to an amount of an ADC effective to treat a disease or disorder in a subject. In the case of cancer, a therapeutically effective amount of ADC can reduce the number of cancer cells, reduce tumor size, inhibit (e.g., slow or stop) tumor metastasis, inhibit (e.g., slow or stop) tumor growth, and/or relieve one or more symptoms. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The term “epitope” refers to the portion of an antigen capable of being recognized and specifically bound by an antibody. When the antigen is a polypeptide, epitopes can be formed from contiguous amino acids or noncontiguous amino acids juxtaposed by tertiary folding of the polypeptide. The epitope bound by an antibody may be identified using any epitope mapping technique known in the art, including X-ray crystallography for epitope identification by direct visualization of the antigen-antibody complex, as well as monitoring the binding of the antibody to fragments or mutated variations of the antigen, or monitoring solvent accessibility of different parts of the antibody and the antigen. Exemplary strategies used to map antibody epitopes include, but are not limited to, array-based oligo-peptide scanning, limited proteolysis, site-directed mutagenesis, high-throughput mutagenesis mapping, hydrogen-deuterium exchange, and mass spectrometry. See, e.g., Gershoni et al. (2007) 21:145-56; and Hager-Braun and Tomer (2005) Expert Rev. Proteomics 2:745-56.

Competitive binding and epitope binning can also be used to determine antibodies sharing identical or overlapping epitopes. Competitive binding can be evaluated using a cross-blocking assay, such as the assay described in “Antibodies, A Laboratory Manual,” Cold Spring Harbor Laboratory, Harlow and Lane (1st edition 1988, 2nd edition 2014). In some embodiments, competitive binding is identified when a test antibody or binding protein reduces binding of a reference antibody or binding protein to a target antigen such as CD73 (e.g., a binding protein comprising CDRs and/or variable domains selected from those identified in Tables 2, 4, and 6), by at least about 50% in the cross-blocking assay (e.g., 50%, 60%, 70%, 80%, 90%, 95%, 99%, 99.5%, or more, or any percentage in between), and/or vice versa. In some embodiments, competitive binding can be due to shared or similar (e.g., partially overlapping) epitopes, or due to steric hindrance where antibodies or binding proteins bind at nearby epitopes. See, e.g., Tzartos, Methods in Molecular Biology (Morris, ed. (1998) vol. 66, pp. 55-66). In some embodiments, competitive binding can be used to sort groups of binding proteins that share similar epitopes, e.g., those that compete for binding can be “binned” as a group of binding proteins that have overlapping or nearby epitopes, while those that do not compete are placed in a separate group of binding proteins that do not have overlapping or nearby epitopes.

The term “Compound 1,” as used herein, refers to the structure of Compound 1 shown below, or a salt thereof:

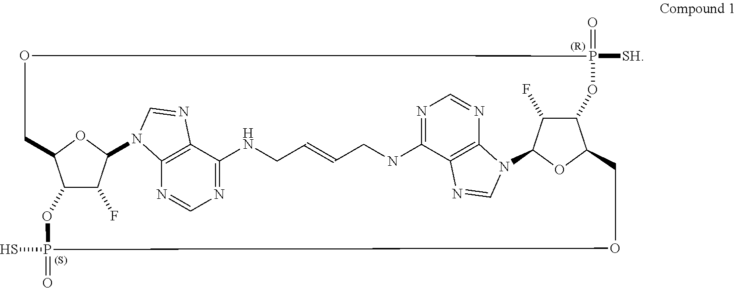

Compound 1 is a macrocycle-bridged STING agonist (MBSA) with a locked bioactive U-shaped conformation of cyclic dinucleotides comprising a transannular macrocyclic bridge between the nucleic acid bases. As used herein, “Compound 1” may include salts of Compound 1, e.g., diammonium salt and/or sodium salt of Compound 1. The term “Compound 1 moiety,” “E7766,” “E7766 agonist moiety,” or “E7766 moiety” refers to the component of an ADC that has the structure of Compound 1, and is attached to the linker of the ADC, e.g., via its N-34 nitrogen, N-39 nitrogen, S-2 sulfur, or S-14 sulfur of the Compound 1 moiety. Compositions and methods of inhibiting tumor growth in patients comprising administering Compound 1 are disclosed in WO 2018/152450, which is incorporated herein by reference in its entirety for all Compound 1 structures and methods of synthesizing those structures.

Atoms in Compound 1, as referenced herein, may be numbered as shown below:

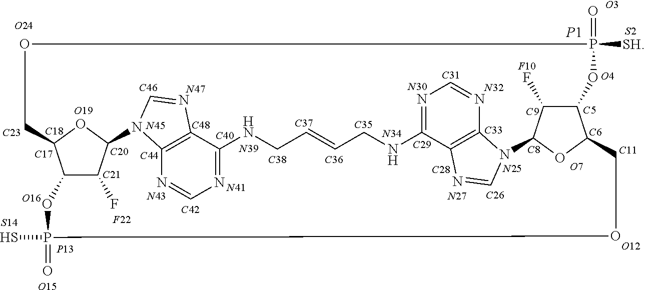

The term “Compound 2,” as used herein, refers to the structure of Compound 2 shown below, or a salt thereof:

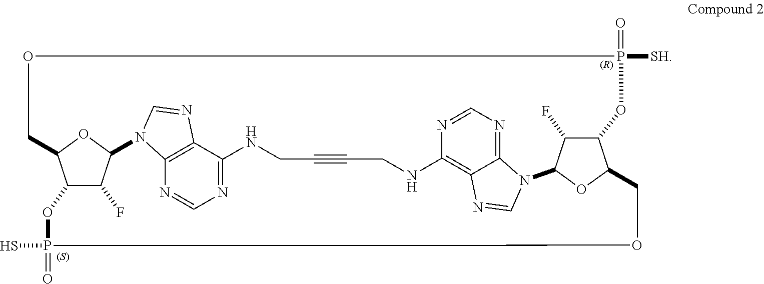

Atoms in Compound 2, as referenced herein, may be numbered as shown below:

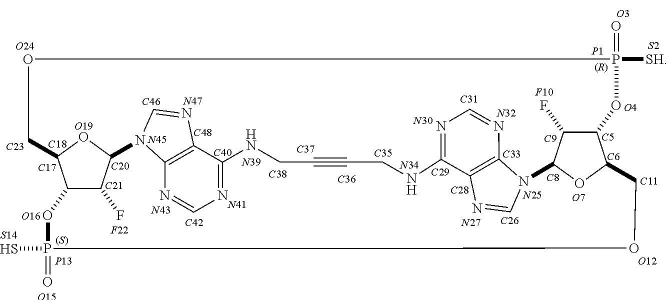

In various embodiments of the disclosure, “N-34 nitrogen,” “N-39 nitrogen,” “S-2 sulfur,” or “S-14 sulfur” may be used to refer to the nitrogen or sulfur atoms in other STING agonists that correspond to the numbered nitrogen or sulfur atoms in Compound 1, regardless of whether the atoms would be numbered otherwise according to the naming convention. In some instances, for compounds of Table 13, e.g., Compound 1, an L-D conjugate with attachment at the N-34 nitrogen may be referred to as “R_{N}” or “RN,” and an L-D conjugate with attachment at the N-39 nitrogen may be referred to as “S_{N}” or “SN.”

“Fcγ receptor,” “Fc-gamma receptor,” or “FcγR” refers to a cell surface protein generally found on immune cells of various types, e.g., neutrophils. The binding of an Fc region of an antibody to an Fcγ receptor may induce different effector functions, for example antibody-dependent cellular cytotoxicity (ADCC) or antibody-dependent cellular phagocytosis (ADCP).

The term “homolog” refers to a molecule which exhibits homology to another molecule, by, for example, having sequences of chemical residues that are the same or similar at corresponding positions.

The terms “IgG1 Fc,” “IgG1 Fc domain” or “IgG1 Fc-containing antibody” as used herein refer to an antibody having at least an IgG1 CH2 and CH3 domain, as identified by SEQ ID NO: 240 and SEQ ID NO: 241, respectively.

“Wild type IgG1 Fc domain” refers to a human IgG1 Fc domain that comprises the amino acid sequence of SEQ ID NO: 242 or a fragment thereof.

The term “inhibit,” or “inhibition of,” as used herein, means to reduce by a measurable amount, and can include but does not require complete prevention or inhibition.

“Internalizing” as used herein in reference to an antibody or antigen-binding fragment refers to an antibody or antigen-binding fragment that is capable of being taken through the cell's lipid bilayer membrane to an internal compartment (i.e., “internalized”) upon binding to the cell, preferably into a degradative compartment in the cell. For example, an internalizing anti-CD73 antibody is one that is capable of being taken into the cell after binding to CD73 on the cell membrane.

The term “KD” refers to the equilibrium dissociation constant of a particular antibody-antigen interaction. KD is calculated by ka/kd. The rate can be determined using standard assays, such as a BIAcore® or ELISA assay.

The term “kₒₙ” or “kₐ” refers to the on-rate constant for association of an antibody to the antigen to form the antibody/antigen complex. The rate can be determined using standard assays, such as a BIAcore® or ELISA assay.

The term “k_{off}” or “k_{d}” refers to the off-rate constant for dissociation of an antibody from the antibody/antigen complex. The rate can be determined using standard assays, such as a BIAcore® or ELISA assay.

A “linker” or “linker moiety” is any chemical moiety that is capable of covalently joining a compound, usually a drug moiety such as a chemotherapeutic agent, to another moiety such as an antibody moiety. Linkers can be susceptible to or substantially resistant to acid-induced cleavage, peptidase-induced cleavage, light-based cleavage, esterase-induced cleavage, and/or disulfide bond cleavage, at conditions under which the compound or the antibody remains active. A “cleavable linker” is any linker that comprises a cleavable moiety and can thus be susceptible to cleavage. A cleavable moiety can be a cleavable peptide moiety. The term “cleavable peptide moiety” refers to any chemical bond linking amino acids (natural or synthetic amino acid derivatives) that can be cleaved by an agent that is present in the intracellular environment.

The use of “or” will mean “and/or” unless the specific context of its use dictates otherwise.

The term “p” or “antibody:drug ratio” or “drug-to-antibody ratio” or “DAR” refers to the number of drug moieties per antibody moiety, i.e., drug loading, or the number of L-D moieties per antibody or antigen-binding fragment (Ab) in ADCs of Formula I. In compositions comprising multiple copies of ADCs of Formula I, “p” refers to the average number of L-D moieties per antibody or antigen-binding fragment, also referred to as average drug loading.

A “pharmaceutical composition” refers to a preparation which is in such form as to permit administration and subsequently provide the intended biological activity of the active ingredient(s) and/or to achieve a therapeutic effect, and which contains no additional components which are unacceptably toxic to a subject to which the formulation would be administered. The pharmaceutical composition may be sterile.

A “pharmaceutical excipient” comprises a material such as an adjuvant, a carrier, pH-adjusting and buffering agents, tonicity adjusting agents, wetting agents, preservative, and the like.

“Pharmaceutically acceptable” means approved or approvable by a regulatory agency of the Federal or a state government, or listed in the U.S. Pharmacopeia or other generally recognized pharmacopeia, for use in animals, and more particularly in humans.

The term “protecting group,” as used herein, refers to any chemical group introduced into a molecule by chemical modification of a functional group to obtain chemoselectivity in a subsequent chemical reaction.

Methods of adding (a process generally referred to as “protecting”) and removing (process generally referred to as “deprotecting”) protecting groups are well-known in the art and available, for example, in P. J. Kocienski, Protecting Groups, 3rd edition (Thieme, 2005), and in Greene and Wuts, Protective Groups in Organic Synthesis, 4th edition (John Wiley & Sons, New York, 2007), both of which are hereby incorporated by reference in their entirety.

Non-limiting examples of useful protecting groups for amines that may be used in this disclosure include monovalent protecting groups, for example, t-butyloxycarbonyl (Boc), benzyl (Bn), 9-fluorenylmethyloxycarbonyl (Fmoc), benzyloxycarbonyl (Cbz), formyl, acetyl (Ac), trifluoroacetyl (TFA), and p-toluenesulfonyl (Ts); and divalent protecting groups, for example, benzylidene, N-phthalimide, N-dithiasuccinimide, N-2,3-diphenylmaleimide, N-2,3-dimethylmaleimide, and N-2,5-dimethylpyrrole.

Non-limiting examples of useful protecting groups for alcohols that may be used in this disclosure include, for example, acetyl (Ac), benzoyl (Bz), benzyl (Bn), β-methoxyethoxymethyl (MEM), dimethoxytrityl (DMT), methoxymethyl (MOM), methoxytrityl (MMT), p-methoxybenzyl (PMB), pivaloyl (Piv), tetrahydropyranyl (THP), trityl (Tr), 4-nitrophenyl carbonate, trimethylsilyl (TMS), triethylsilyl (TES), triisopropylsilyl (TIPS), t-butyldimethylsilyl (TBS), and t-butyldiphenylsilyl (TBDPS).

Non-limiting examples of useful protecting groups for carboxylic acids that may be used in this disclosure include, for example, methyl or ethyl esters, substituted alkyl esters such as 9-fluorenylmethyl, methoxymethyl (MOM), tetrahydropyranyl (THP), tetrahydrofuranyl, β-methoxyethoxymethyl (MEM), 2-(trimethylsilyl)ethoxymethyl (SEM), benzyloxymethyl (BOM), acetyl (Ac), phenacyl, substituted phenacyl esters, t-butyl, allyl, phenyl (Ph), silyl esters, benzyl and substituted benzyl esters, 2,6-dialkylphenyl, and pentafluorophenyl (PFP).

Non-limiting examples of amine bases that may be used in this disclosure include, for example, 1,8-diazabicyclo[5.4.0]undec-7-ene (DBU), N-methylmorpholine (NMM), triethylamine (Et₃N; TEA), diisopropylethyl amine (i-Pr₂EtN; DIPEA), pyridine, 2,2,6,6-tetramethylpiperidine, 1,5,7-triazabicyclo[4.4.0]dec-5-ene (TBD), 7-methyl-1,5,7-triazabicyclo[4.4.0]dec-5-ene (MTBD), t-Bu-tetramethylguanidine, 1,5-diazabicyclo[4.3.0]non-5-ene (DBN), lithium bis(trimethylsilyl)amide (LiHMDS), and potassium bis(trimethylsilyl)amide (KHMDS).

Non-limiting examples of carbonate bases that may be used in this disclosure include, for example, sodium carbonate (Na₂CO₃), potassium carbonate (K₂CO₃), cesium carbonate (Cs₂CO₃), lithium carbonate (Li₂CO₃), sodium bicarbonate (NaHCO₃), and potassium bicarbonate (KHCO₃).

Non-limiting examples of phosphate bases that may be used in this disclosure include, for example, sodium phosphate tribasic (Na₃PO₄), potassium phosphate tribasic (K₃PO₄), potassium phosphate dibasic (K₂HPO₄), and potassium phosphate monobasic (KH₂PO₄).

Non-limiting examples of acids that may be used in this disclosure include, for example, acetic acid (AcOH), trifluoroacetic acid (TFA), hydrochloric acid (HCl), camphorsulfonic acid (CSA), methanesulfonic acid (MsOH), formic acid (FA), phosphoric acid (H₃PO₄), and sulfuric acid (H₂SO₄).

Non-limiting examples of peptide coupling reagents include, for example, N,N′-dicyclohexylcarbodiimide (DCC), 1-ethyl-3-[3-(dimethylamino)propyl]carbodiimide (EDCI), 4-(4,6-dimethoxy-1,3,5-triazin-2-yl)-4-methylmorpholin-4-ium chloride (DMT-MM), 1-ethoxycarbonyl-2-ethoxy-1,2-dihydroquinoline (EEDQ), 1-[bis(dimethylamino)methylene]-1H-1,2,3-triazolo[4,5-b]pyridinium 3-oxid hexafluorophosphate (HATU), 1-hydroxybenzotriazole (HOBT), and N,N,N,N-tetramethyl-O—(N-succinimidyl)uronium tetrafluoroborate (TSTU).

For amino acid sequences, sequence identity and/or similarity may be determined using standard techniques known in the art, including, but not limited to, the local sequence identity algorithm of Smith and Waterman (1981) Adv. Appl. Math. 2:482, the sequence identity alignment algorithm of Needleman and Wunsch (1970) J. Mol. Biol. 48:443, the search for similarity method of Pearson and Lipman (1988) Proc. Nat. Acad. Sci. USA 85:2444, computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al. (1984) Nucl. Acid Res. 12:387-95, preferably using the default settings, or by inspection. Preferably, percent identity is calculated by FastDB based upon the following parameters: mismatch penalty of 1; gap penalty of 1; gap size penalty of 0.33; and joining penalty of 30. See “Current Methods in Sequence Comparison and Analysis,” Macromolecule Sequencing and Synthesis, Selected Methods and Applications, pp. 127-149 (1988), Alan R. Liss, Inc.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle (1987) J. Mol. Evol. 35:351-60; the method is similar to that described by Higgins and Sharp (1989) CABIOS 5:151-3. Useful PILEUP parameters including a default gap weight of 3.00, a default gap length weight of 0.10, and weighted end gaps.

Another example of a useful algorithm is the BLAST algorithm, described in: Altschul et al. (1990) J. Mol. Biol. 215:403-10; Altschul et al. (1997) Nucleic Acids Res. 25:3389-402; and Karin et al. (1993) Proc. Natl. Acad. Sci. USA 90:5873-87. A particularly useful BLAST program is the WU-BLAST-2 program which was obtained from Altschul et al. (1996) Methods in Enzymology 266:460-80. WU-BLAST-2 uses several search parameters, most of which are set to the default values. The adjustable parameters are set with the following values: overlap span=1, overlap fraction=0.125, word threshold (T)=II. The HSP S and HSP S2 parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al. (1993) Nucl. Acids Res. 25:3389-402. Gapped BLAST uses BLOSUM-62 substitution scores; threshold T parameter set to 9; the two-hit method to trigger ungapped extensions, charges gap lengths of k a cost of 10+k; Xu set to 16, and Xg set to 40 for database search stage and to 67 for the output stage of the algorithms. Gapped alignments are triggered by a score corresponding to about 22 bits.

Generally, proteins disclosed herein and variants thereof (e.g., variants that retain function of the original protein), including variants of CD73, and variants of antibody variable domains (including individual variant CDRs), have amino acid homology, similarity, or identity of at least 80%, and more typically homologies or identities of at least 85%, 90%, 91%, 92%, 93%, 94%, 95%, 96%, 97%, 98%, 99%, and almost 100% or 100%.

In a similar manner, “percent (%) nucleic acid sequence identity” with respect to the nucleic acid sequence of the antibodies and other proteins identified herein is defined as the percentage of nucleotide residues in a candidate sequence that are identical with the nucleotide residues in the coding sequence of the antigen binding protein. A specific method uses the BLASTN module of WU-BLAST-2 set to the default parameters, with overlap span and overlap fraction set to 1 and 0.125, respectively.

The terms “subject” and “patient” are used interchangeably herein to refer to any animal, such as any mammal, including but not limited to, humans, non-human primates, rodents, and the like. In some embodiments, the mammal is a mouse. In some embodiments, the mammal is a rabbit. In some embodiments, the mammal is a human.

The term “target-negative” or “target antigen-negative” refers to the absence of target antigen expression by a cell or tissue. The term “target-positive” or “target antigen-positive” refers to the presence of target antigen expression. For example, a cell or a cell line that does not express a target antigen may be described as target-negative, whereas a cell or cell line that expresses a target antigen may be described as target-positive.

As used herein, the term “solvent” refers to any liquid in which the product is at least partially soluble (solubility of product >1 g/L).

As used herein, the term “isomer” refers to compounds with identical molecular formula but distinct spatial arrangement of atoms or bonds. Isomers include stereoisomers, cis-trans isomers, atropisomers, and tautomers.

As used herein, the term “stereoisomer” refers to both enantiomers and diastereomers.

It will be appreciated that certain compounds of this invention may exist as separate stereoisomers or enantiomers and/or mixtures of those stereoisomers or enantiomers. As used in the chemical structures disclosed herein, a “wedge” ( [symbol] ), “bold” (

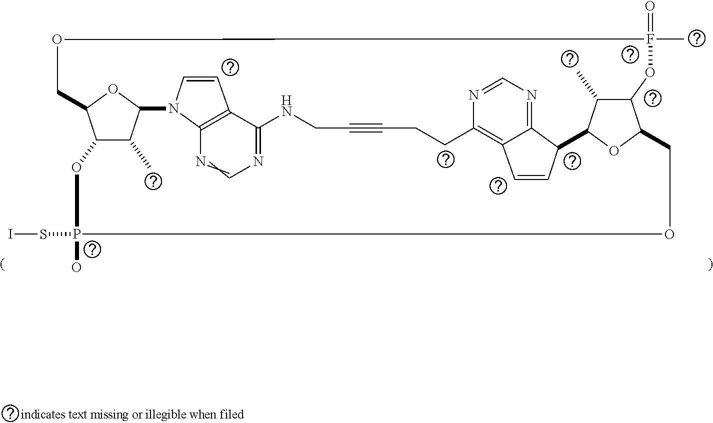

or “hash” ( [symbol] ) bond to a stereogenic atom indicates a chiral center of known absolute stereochemistry (i.e., one stereoisomer). As used herein, a stereogenic atom that is notated with an (R) or (S) indicates the stereochemical designation of the stereogenic atom under the Cahn-Ingold-Prelog convention. As used in the chemical structures disclosed herein, a — (“straight”) bond to a stereogenic atom indicates where there is a mixture (e.g., a racemate or enrichment). As used herein, two — (“straight”) bonds to a double-bonded carbon indicates that the double bond possesses the E/Z stereochemistry as drawn.

Certain compounds disclosed herein may exist as tautomers and both tautomeric forms are intended, even though only a single tautomeric structure is depicted.

The disclosure also provides processes for preparing salts of the compounds of the disclosure.

A salt of a compound of this disclosure is formed between an acid and basic group(s) of the compound, such as an amino functional group, or a base and acidic group(s) of the compound, such as a carboxyl functional group. Depending on the ratio of the basic or acidic group(s) in the compound to the valence of the acid or base, one compound may form salt with one or more molecular units of the acid/base, or multiple units of the compound may form salt with one unit of the acid/base. In some embodiments, the salt is a sodium salt. In some embodiments, the salt is a diammonium salt. In some embodiments, the salt is a dialkylammonium salt. In some embodiments, the salt is a bis(triethylammonium) salt.

The term “stimulator of interferon genes” or “STING,” as used herein, refers to any native form of human STING. The term encompasses full-length STING (e.g., NCBI Reference Sequence: NP_938023.1; SEQ ID NO: 243), as well as any form of human STING that results from cellular processing. The term also encompasses naturally occurring variants of STING, including but not limited to splice variants, allelic variants, and isoforms. STING can be isolated from a human, or may be produced recombinantly or by synthetic methods.

The term “tumor microenvironment (TME)” refers to a local disease niche, in which a tumor (e.g., a solid tumor) resides in vivo. The TME may comprise disease-associated molecular signature (i.e., a set of chemokines, cytokines, etc.), disease-associated cell populations (such as tumor-associated macrophages (TAM), and cancer-associated fibroblasts (CAF), myeloid-derived suppressor cells (MDSC), etc.), as well as disease-associated extracellular matrix (ECM) environments (e.g., alterations in ECM components and/or structure).

As used herein, “to treat” or “therapeutic” and grammatically related terms, refer to any improvement of any consequence of disease, such as prolonged survival, less morbidity, and/or a lessening of side effects which are the byproducts of an alternative therapeutic modality. As is readily appreciated in the art, full eradication of disease is preferred but albeit not a requirement for a treatment act. “Treatment” or “treat,” as used herein, refers to the administration of a described ADC to a subject, e.g., a patient. The treatment can be to cure, heal, alleviate, relieve, alter, remedy, ameliorate, palliate, improve, or affect the disorder, the symptoms of the disorder, or the predisposition toward the disorder, e.g., a cancer.

As used herein, the term “unsaturated” means that a moiety has one or more units of unsaturation.

### Anti-CD73 Antibodies and Antigen-Binding Fragments

The present disclosure provides antibodies that specifically bind to CD73 and may be used alone, e.g., formulated as therapeutic or diagnostic antibody compositions, e.g., for use in treating or detecting CD73-expressing cancers. The antibodies may be provided packaged or prepared for therapeutic use as antibodies, antigen-binding fragments thereof, or as portions of ADCs. Antibodies include, but are not limited to, those listed in Table 1, as well as antibodies comprising CDRs and/or variable domains from the listed antibodies.

The antibodies disclosed herein may bind to CD73 with a binding affinity (KD) of ≤1 mM, ≤100 nM, or ≤10 nM, or any amount in between, as measured by, e.g., BIAcore® analysis. In some embodiments, the KD is 500 pM to 1 nM, or 1 nM to 10 nM. In some embodiments, the KD is ≤10 nM, ≤5 nM, ≤1 nM, or ≤0.5 nM.

In some embodiments, the antibodies are four-chain antibodies (also referred to as an immunoglobulin), comprising two heavy chains and two light chains. In some embodiments, the antibodies are two-chain half bodies (one light chain and one heavy chain), or antigen-binding fragments of an immunoglobulin.

Any antibody or antigen-binding fragment described herein may comprise part of a bispecific or multi-specific binding construct, e.g., a construct that can bind to at least one different antigen or additional epitope on CD73.

In some embodiments, the antibodies are internalizing antibodies or internalizing antigen-binding fragments thereof. In some embodiments, the internalizing antibodies bind to CD73 expressed on the surface of a cell and enter the cell upon or after binding. In some embodiments, the drug moiety of the ADC is released from the antibody moiety of the ADC after the ADC enters and is present in a cell expressing CD73 (i.e., after the ADC has been internalized). In some embodiments, the internalizing antibodies bind to CD73 expressed on the cell surface of a cell and the cell is subsequently phagocytosed (e.g., antibody-dependent cellular phagocytosis occurs). In some embodiments, the drug moiety of the ADC is released from the antibody moiety of the ADC after the ADC enters and is present in the phagocytic cell (e.g., macrophage, dendritic cell).

The antibodies disclosed herein that specifically bind a CD73 protein may comprise three heavy chain CDRs (HCDR1, HCDR2, and HCDR3) having amino acid sequences selected from the HC CDRs listed in Table 2, infra, as defined by the Kabat numbering system, and three light chain CDRs (LCDR1, LCDR2, and LCDR3) having amino acid sequences selected from the LC CDRs listed in Table 2, infra, as defined by the Kabat numbering system. In some embodiments, the antibodies comprise three heavy chain CDRs (HCDR1, HCDR2, and HCDR3) having amino acid sequences selected from the HC CDRs listed in Table 4, infra, as defined by the IMGT numbering system, and three light chain CDRs (LCDR1, LCDR2, and LCDR3) having amino acid sequences selected from the LC CDRs listed in Table 4, infra, as defined by the IMGT numbering system.

In some embodiments, an antibody disclosed herein comprises a VH domain having an amino acid sequence of SEQ ID NOs: 49, 52, 55, and 58 listed in Table 6, infra. In some embodiments, the antibody comprises a VL domain having an amino acid sequence of SEQ ID NOs: 50, 51, 53, 54, 56, 57, 59, and 60 listed in Table 6, infra.

In some embodiments, an antigen-binding fragment disclosed retains CD73 binding. In some embodiments, the antigen binding fragment retains CD73 binding by comprising three heavy chain CDRs (HCDR1, HCDR2, and HCDR3) comprising amino acid sequences selected from the HC CDRs listed in Table 2, infra, as defined by the Kabat numbering system, and three light chain CDRs (LCDR1, LCDR2, and LCDR3) comprising amino acid sequences selected from the LC CDRs listed in Table 2, infra, as defined by the Kabat numbering system. In some embodiments, the antigen binding fragment comprises three heavy chain CDRs (HCDR1, HCDR2, and HCDR3) comprising amino acid sequences selected from the HC CDRs listed in Table 4, infra, as defined by the IMGT numbering system, and three light chain CDRs (LCDR1, LCDR2, and LCDR3) comprising amino acid sequences selected from the LC CDRs listed in Table 4, infra, as defined by the IMGT numbering system. In some embodiments, the antigen-binding fragments disclosed herein may retain CD73 binding by comprising a VH domain comprising an amino acid sequence of SEQ ID NOs: 49, 52, 55, and 58 listed in Table 6, infra, and a VL domain comprising an amino acid sequence of SEQ ID NOs: 53, 54, 56, 57, 59, and 60 listed in Table 6, infra.

In some embodiments, the antibodies disclosed herein may comprise an IgG constant domain, e.g., an IgG1, IgG2, or IgG4 domain, or an IgG1 domain that has been modified to reduce binding to an Fc receptor, e.g., an Fcγ receptor (FcγR) as compared to a wild-type constant domain-containing (e.g., a wild-type IgG1-containing) antibody. Reduced binding to an Fc receptor, e.g., to an FcγR, can be measured as a comparison to the binding of the wild-type antibody without the modification to the same receptor. Reduced binding may be by at least about 5-fold, and preferably at least 10-fold, at least 50-fold, at least 100-fold, or at least 1000-fold as compared to the antibody containing the wild-type constant domain. Reduced binding may be measured using any assay known in the art. For example, reduced binding may be measured using a fluorescence resonance energy transfer (FRET) assay. In some embodiments, the modified IgG constant domain is modified by Fc engineering and/or glycan modification, e.g., deglycosylation.

In some embodiments, an antibody disclosed herein may comprise an IgG1 comprising the mutations N297Q, N297A, L234G/L235G, L234A/L235A, L234A/L235A/D265S, L234A/L235A/P329G, L234A/L235A/P238S/H268Q/K274Q, L235G/G236R, G236R/L328R, and/or L234S/L235T/G236R, according to the EU numbering. In some embodiments, an antibody disclosed herein may comprise an IgG1 comprising the mutations L234A, L235A, P238S, H268Q, and/or K274Q (e.g., comprising all of those mutations) according to the EU numbering (Wang et al. (2017) Protein Cell 9(1):63-73; Vafa et al. (2014) Methods 1; 65(1):114-26; Tam et al. (2017) Antibodies 1; 6(3):12). Without being bound by theory, these mutations may reduce binding of the antibody to a Fcγ receptor (FcγR), which may reduce non-antigen mediated uptake of antibodies or ADCs by immune cells, such as neutrophils, thus reducing neutropenia. Reduced neutropenia may be measured using any assay known in the art.

In some embodiments, an antibody that specifically binds a CD73 protein comprises a heavy chain having an amino acid sequence of SEQ ID NOs: 61 to 64, 67 to 70, 73 to 76, and 79 to 82 listed in Table 8, infra and/or comprising a set of CDRs and/or a variable domain from the amino acid sequences in Table 8. In some embodiments, an antibody that specifically binds a CD73 protein comprises a light chain having an amino acid sequence of SEQ ID NOs: 65, 66, 71, 72, 77, 78, 83, and 84 listed in Table 8, infra and/or comprising a set of CDRs and/or a variable domain from the amino acid sequences in Table 8.

Amino acid and nucleic acid sequences of exemplary antibodies of the present disclosure are set forth in Tables 1-9.

TABLE 1
Antibodies

| | | | IgG | SEQ ID | Amino acid sequence of |
|---|---|---|---|---|---|
| mAb | Type | Target | chain | NO | variable region |
| mu190K12 | mouse | human | Heavy | 280 | QAYLQQSGAALVRPGASVKMSCKSSGYTFT |
| | | CD73 | chain | | TYNIHWVKQTPRQGLEWIGAIFPGNGHISY |
| | | | | | NQKFKGKATLTVDKSSNTAYMQLSSLTSED |
| | | | | | SAVYFCARGDYDESYFDYWGQGTTLTVSSA |
| | | | | | STKGPSVFPLAPSSKSTSGGTAALGCLVKD |
| | | | | | YFPEPVTVSWNSGALTSGVHTFPAVLQSSG |
| | | | | | LYSLSSVVTVPSSSLGTQTYICNVNHKPSN |
| | | | | | TKVDKKVEPKSCDKTHTCPPCPAPELLGGP |
| | | | | | SVFLFPPKPKDTLMISRTPEVTCVVVDVSH |
| | | | | | EDPEVKFNWYVDGVEVHNAKTKPREEQYNS |
| | | | | | TYRVVSVLTVLHQDWLNGKEYKCKVSNKAL |
| | | | | | PAPIEKTISKAKGQPREPQVYTLPPSRDEL |
| | | | | | TKNQVSLTCLVKGFYPSDIAVEWESNGQPE |
| | | | | | NNYKTTPPVLDSDGSFFLYSKLTVDKSRWQ |
| | | | | | QGNVFSCSVMHEALHNHYTQKSLSLSPGK |
| mu190K12 | mouse | human | Light | 86 | DIQMTQSSSSFSVSLGDRVTLSCKASEGIY |
| | | CD73 | chain | | NRLAWYQQKPGNAPRLLISGVISLETGVPS |
| | | | | | RFSGSGSGKDYTLSITSLQTEDVATYYCQQ |
| | | | | | YWSIPWTFGGGNKLEINRTVAAPSVFIFPP |
| | | | | | SDEQLKSGTASVVCLLNNFYPREAKVQWKV |
| | | | | | DNALQSGNSQESVTEQDSKDSTYSLSSTLT |
| | | | | | LSKADYEKHKVYACEVTHQGLSSPVTKSFN |
| | | | | | RGEC |
| mu366F5 | mouse | human | Heavy | 87 | QVQLQQSGAELVKPGASVKISCKASGYAFR |
| | | CD73 | chain | | SYWVNWVKQRPGKGLEWIGQIFPGDGDINY |
| | | | | | SGSFKGKATLTADKSSSTAYMQLSSLTSED |
| | | | | | SAVYFCARMDFYSWFPYWGQGTLVTVSAAS |
| | | | | | TKGPSVFPLAPSSKSTSGGTAALGCLVKDY |
| | | | | | FPEPVTVSWNSGALTSGVHTFPAVLQSSGL |
| | | | | | YSLSSVVTVPSSSLGTQTYICNVNHKPSNT |
| | | | | | KVDKKVEPKSCDKTHTCPPCPAPELLGGPS |
| | | | | | VFLFPPKPKDTLMISRTPEVTCVVVDVSHE |
| | | | | | DPEVKFNWYVDGVEVHNAKTKPREEQYNST |
| | | | | | YRVVSVLTVLHQDWLNGKEYKCKVSNKALP |
| | | | | | APIEKTISKAKGQPREPQVYTLPPSRDELT |
| | | | | | KNQVSLTCLVKGFYPSDIAVEWESNGQPEN |
| | | | | | NYKTTPPVLDSDGSFFLYSKLTVDKSRWQQ |
| | | | | | GNVFSCSVMHEALHNHYTQKSLSLSPGK |
| mu366F5 | mouse | human | Light | 88 | DIVMTQSPSSLSVSAGEKVTMSCKSSQSLL |
| | | CD73 | chain | | NSGNQKNDLAWFQQKPGQPPKLLIYGASTR |
| | | | | | ESGVPDRFTGSGSGTDFTLTISSVQAEDLA |
| | | | | | VYYCQNDHSYPLTFGTGTKLELKRTVAAPS |
| | | | | | VFIFPPSDEQLKSGTASVVCLLNNFYPREA |
| | | | | | KVQWKVDNALQSGNSQESVTEQDSKDSTYS |
| | | | | | LSSTLTLSKADYEKHKVYACEVTHQGLSSP |
| | | | | | VTKSENRGEC |
| rb23P11 | rabbit | human | Heavy | 89 | QELKESGGGLVTPGGTLTLTCTASGFSLSN |
| | | CD73 | chain | | YYINWVRQAPGKGLEYIGFIPMYGTTHYAN |
| | | | | | WAKGRFTISRTSTTVDLKMASLTASDTATY |
| | | | | | FCARGIASMFYPSIWGPGTLVTVSSASTKG |
| | | | | | PSVFPLAPSSKSTSGGTAALGCLVKDYFPE |
| | | | | | PVTVSWNSGALTSGVHTFPAVLQSSGLYSL |
| | | | | | SSVVTVPSSSLGTQTYICNVNHKPSNTKVD |
| | | | | | KKVEPKSCDKTHTCPPCPAPELLGGPSVEL |
| | | | | | FPPKPKDTLMISRTPEVTCVVVDVSHEDPE |
| | | | | | VKFNWYVDGVEVHNAKTKPREEQYNSTYRV |
| | | | | | VSVLTVLHQDWLNGKEYKCKVSNKALPAPI |
| | | | | | EKTISKAKGQPREPQVYTLPPSRDELTKNQ |
| | | | | | VSLTCLVKGFYPSDIAVEWESNGQPENNYK |
| | | | | | TTPPVLDSDGSFFLYSKLTVDKSRWQQGNV |
| | | | | | FSCSVMHEALHNHYTQKSLSLSPGK |
| rb23P11 | rabbit | human | Light | 90 | DVVMTQTPSSTSAAVGGAVTIKCQASEDIH |
| | | CD73 | chain | | GYLAWYQQKPGQPPKLLIYYASTLASGVSS |
| | | | | | RFKGSGSGTEYTLTISGVQREDAATYYCLG |
| | | | | | DGSTSGITFGGGTEFEILRTVAAPSVFIFP |
| | | | | | PSDEQLKSGTASVVCLLNNFYPREAKVQWK |
| | | | | | VDNALQSGNSQESVTEQDSKDSTYSLSSTL |
| | | | | | TLSKADYEKHKVYACEVTHQGLSSPVTKSF |
| | | | | | NRGEC |
| rb2406 | rabbit | human | Heavy | 91 | QSVEESGGRLVTPGTPLTLTCTVSGIDLSS |
| | | CD73 | chain | | STMAWVRQAPGKGLEYIGIIASSGSAYYAG |
| | | | | | WAKGRFTISKTSSTTVDLKITSPTTEDTAT |
| | | | | | YFCVRQVPGYNDNRYVWGPGTLVTVSSAST |
| | | | | | KGPSVFPLAPSSKSTSGGTAALGCLVKDYF |
| | | | | | PEPVTVSWNSGALTSGVHTFPAVLQSSGLY |
| | | | | | SLSSVVTVPSSSLGTQTYICNVNHKPSNTK |
| | | | | | VDKKVEPKSCDKTHTCPPCPAPELLGGPSV |
| | | | | | FLFPPKPKDTLMISRTPEVTCVVVDVSHED |
| | | | | | PEVKFNWYVDGVEVHNAKTKPREEQYNSTY |
| | | | | | RVVSVLTVLHQDWLNGKEYKCKVSNKALPA |
| | | | | | PIEKTISKAKGQPREPQVYTLPPSRDELTK |
| | | | | | NQVSLTCLVKGFYPSDIAVEWESNGQPENN |
| | | | | | YKTTPPVLDSDGSFFLYSKLTVDKSRWQQG |
| | | | | | NVFSCSVMHEALHNHYTQKSLSLSPGK |
| rb2406 | rabbit | human | Light | 92 | AIVMTQTPSSVSAAVGGTVTIHCQASESVY |
| | | CD73 | chain | | NANSCSWFQQKPGQRPKLLIYDASDLASGV |
| | | | | | PSRFRGSGSGTQFTLTINNVQREDVATYFC |
| | | | | | AGYKGNGDAAFGGGTELEILRTVAAPSVFI |
| | | | | | FPPSDEQLKSGTASVVCLLNNFYPREAKVQ |
| | | | | | WKVDNALQSGNSQESVTEQDSKDSTYSLSS |
| | | | | | TLTLSKADYEKHKVYACEVTHQGLSSPVTK |
| | | | | | SFNRGEC |

Bolded text indicates amino acid positions corresponding to CDR sequences according to the Kabat system; underlined text indicates amino acid positions corresponding to CDR sequences according to the IMGT system. Text that is neither bolded nor underlined corresponds to the mouse- or rabbit- specific framework regions.

In some embodiments, the antibodies listed in Table 1 can be humanized by replacing the mouse-specific framework regions with human-specific framework regions. In some embodiments, the antibodies listed in Table 1 can be humanized by replacing the rabbit-specific framework regions with human-specific framework regions. In some embodiments, a variant of a humanized antibody comprises three heavy chain CDRs (HCDR1, HCDR2, and HCDR3) selected from the CDR sequences listed in Table 2, infra, according to the Kabat system. For example, a humanized variant of 190K12 may comprise three heavy chain CDRs, wherein the HCDR1 comprises SEQ ID NO: 1, the HCDR2 comprises or SEQ ID NO: 2, and the HCDR3 comprises SEQ ID NO: 3. In some embodiments, a variant of a humanized antibody comprises three light chain CDRs (LCDR1, LCDR2, and LCDR3) selected from the CDR sequences listed in Table 2, infra, according to the Kabat system. For example, a humanized variant of 190K12 may comprise three light chain CDRs, wherein the LCDR1 comprises SEQ ID NO: 4, the LCDR2 comprises SEQ ID NO: 5, and the LCDR3 comprises SEQ ID NO: 6.

In some embodiments, a variant of a humanized antibody comprises three heavy chain CDRs (HCDR1, HCDR2, and HCDR3) selected from the CDR sequences listed in Table 4, infra, according to the IMGT system. For example, a humanized variant of 190K12 may comprise three heavy chain CDRs, wherein the HCDR1 comprises SEQ ID NO: 7, the HCDR2 comprises SEQ ID NO: 8, and the HCDR3 comprises SEQ ID NO: 9. In some embodiments, a variant of a humanized antibody comprises three light chain CDRs (LCDR1, LCDR2, and LCDR3) selected from the CDR sequences listed in Table 4, infra, according to the IMGT system. For example, a humanized variant of 190K12 may comprise three light chain CDRs, wherein the LCDR1 comprises SEQ ID NO: 10, the LCDR2 comprises SEQ ID NO: 11, and the LCDR3 comprises SEQ ID NO: 12.

TABLE 2
Amino acid sequences of mAb Kabat CDRs

| | | | |
|---|---|---|---|
| | | SEQ | |
| | IgG | ID | |
| Parent mAb | chain | NO | Amino acid sequence |

Heavy chain CDRs

| | | | |
|---|---|---|---|
| 190K12 | HC CDR1 | 1 | TYNMH |
| | HC CDR2 | 2 | AIFPGNGHISYAQKFQG |
| | HC CDR3 | 3 | GDYDESYFDY |
| 366F5 | HC CDR1 | 13 | SYWIS |
| | HC CDR2 | 14 | QIFPGDGDINYAQKFQG |
| | HC CDR3 | 15 | MDFYSWFPY |
| 23P11 | HC CDR1 | 25 | NYYIN |
| | HC CDR2 | 26 | FIPMYGTTHYADSVKG |
| | HC CDR3 | 27 | GIASMFYPSI |
| 2406 | HC CDR1 | 37 | SSTMA |
| | HC CDR2 | 38 | IIASSGSAYYAGWAKG |
| | HC CDR3 | 39 | QVPGYNDNRYV |

Light chain CDRs

| | | | |
|---|---|---|---|
| 190K12 | LC CDR1 | 4 | KASEGIYNRLA |
| | LC CDR2 | 5 | GVISLET |
| | LC CDR3 | 6 | QQYWSIPWT |
| 366F5 | LC CDR1 | 16 | KSSQSLLNSGNQKNDLA |
| | LC CDR2 | 17 | GASTRES |
| | LC CDR3 | 18 | QNDHSYPLT |
| 23P11 | LC CDR1 | 28 | QASEDIHGYLA |
| | LC CDR2 | 29 | YASTLOS |
| | LC CDR3 | 30 | LGDGSTSGIT |
| 2406 | LC CDR1 | 40 | QASESVYNANSCS |
| | LC CDR2 | 41 | DASDLAS |
| | LC CDR3 | 42 | AGYKGNGDAA |

TABLE 3
Nucleic acid sequences encoding mAB Kabat CDRs

| | | | |
|---|---|---|---|
| Parent mAb | IgG chain | SEQ ID NO | Nucleic acid sequence |

Heavy chain CDRs

| | | | |
|---|---|---|---|
| 190K12 | HC CDR1 | 127 | ACGTACAACATGCAT |
| | HC CDR2 | 128 | GCCATTTTTCCCGGAAACGGCCACATCTCGTAC |
| | | | GCCCAAAAGTTCCAGGGC |
| | HC CDR3 | 129 | GGGGATTACGACGAAAGCTACTTCGACTAC |
| 366F5 | HC CDR1 | 142 | TCGTACTGGATCTCC |
| | HC CDR2 | 143 | CAGATCTTTCCTGGGGACGGCGACATTAACTAC |
| | | | GCGCAGAAGTTCCAGGGC |
| | HC CDR3 | 144 | ATGGACTTCTACTCCTGGTTCCCGTAC |
| 23P11 | HC CDR1 | 154 | AACTACTACATCAAC |
| | HC CDR2 | 155 | TTCATCCCTATGTACGGCACCACACACTACGCC |
| | | | AACTGGGCCAAGGGC |
| | HC CDR3 | 156 | GGAATCGCCAGCATGTTCTACCCTTCTATC |
| 2406 | HC CDR1 | 166 | AGCTCTACAATGGCC |
| | HC CDR2 | 167 | ATCATTGCCAGCAGCGGCTCTGCCTATTATGCC |
| | | | GGATGGGCCAAGGGC |
| | HC CDR3 | 168 | CAGGTGCCCGGCTACAACGACAACAGATATGTG |

Light chain CDRs

| | | | |
|---|---|---|---|
| 190K12 | LC CDR1 | 130 | AAAGCCTCCGAGGGGATCTACAACAGACTCGCC |
| | LC CDR2 | 131 | GGAGTGATCTCCCTGGAAACT |
| | | 132 | GGCGTGATCTCTCTGGAAACA |
| | LC CDR3 | 133 | CAGCAGTACTGGTCGATTCCGTGGACT |
| | | 134 | CAGCAGTACTGGTCTATCCCCTGGACC |
| 366F5 | LC CDR1 | 145 | AAATCCAGCCAGTCGCTGCTGAACTCGGGGAAC |
| | | | CAGAAGAATGATCTGGCC |
| | LC CDR2 | 146 | GGAGCATCAACTCGCGAATCC |
| | LC CDR3 | 147 | CAGAACGACCACTCCTATCCGCTGACT |
| 23P11 | LC CDR1 | 157 | CAGGCCAGCGAGGACATCCACGGCTATCTGGCC |
| | LC CDR2 | 158 | TACGCCTCTACACTGGCCAGC |
| | LC CDR3 | 159 | CTCGGAGATGGCAGCACCAGCGGCATCACA |
| 2406 | LC CDR1 | 169 | CAGGCCAGCGAGAGCGTGTACAACGCCAACAGC |
| | | | TGTAGC |
| | LC CDR2 | 170 | GATGCCTCTGATCTGGCCAGC |
| | LC CDR3 | 171 | GCCGGCTACAAAGGCAATGGCGACGCCGCT |

TABLE 4
Amino acid sequences of mAb IMGT CDRs

| | | | |
|---|---|---|---|
| Parent mAb | IgG chain | SEQ ID NO | Amino acid sequence |

Heavy chain CDRs

| | | | |
|---|---|---|---|
| 190K12 | HC CDR1 | 7 | GYTFTTYN |
| | HC CDR2 | 8 | IFPGNGHI |
| | HC CDR3 | 9 | ARGDYDESYFDY |
| 366F5 | HC CDR1 | 19 | GYAFRSYW |
| | HC CDR2 | 20 | IFPGDGDI |
| | HC CDR3 | 21 | ARMDFYSWFPY |
| 23P11 | HC CDR1 | 31 | GFSLSNYY |
| | HC CDR2 | 32 | IPMYGTT |
| | HC CDR3 | 33 | ARGIASMFYPSI |
| 2406 | HC CDR1 | 43 | AGYKGNGDAA |
| | HC CDR2 | 44 | IASSGSA |
| | HC CDR3 | 45 | VRQVPGYNDNRYV |

Light chain CDRs

| | | | |
|---|---|---|---|
| 190K12 | LC CDR1 | 10 | EGIYNR |
| | LC CDR2 | 11 | GVI |
| | LC CDR3 | 12 | QQYWSIPWT |
| 366F5 | LC CDR1 | 22 | QSLLNSGNQKND |
| | LC CDR2 | 23 | GAS |
| | LC CDR3 | 24 | QNDHSYPLT |
| 23P11 | LC CDR1 | 34 | EDIHGY |
| | LC CDR2 | 35 | YAS |
| | LC CDR3 | 36 | LGDGSTSGIT |
| 2406 | LC CDR1 | 46 | ESVYNANS |
| | LC CDR2 | 47 | DAS |
| | LC CDR3 | 48 | AGYKGNGDAA |

TABLE 5
Nucleic acid sequences encoding mAB IMGT CDRs

| | | | |
|---|---|---|---|
| Parent mAb | IgG chain | SEQ ID NO | Nucleic acid sequence |

Heavy chain CDRs

| | | | |
|---|---|---|---|
| 190K12 | HC CDR1 | 135 | GGATACACCTTCACCACGTACAAC |
| | HC CDR2 | 136 | ATTTTTCCCGGAAACGGCCACATC |
| | HC CDR3 | 137 | GCGCGGGGGGATTACGACGAAAGCTACTTCGAC |
| | | | TAC |
| 366F5 | HC CDR1 | 148 | GGATACGCATTCCGGTCGTACTGG |
| | HC CDR2 | 149 | ATCTTTCCTGGGGACGGCGACATT |
| | HC CDR3 | 150 | GCTCGGATGGACTTCTACTCCTGGTTCCCGTAC |
| 23P11 | HC CDR1 | 160 | GGCTTCAGCCTGAGCAACTACTAC |
| | HC CDR2 | 161 | ATCCCTATGTACGGCACCACA |
| | HC CDR3 | 162 | GCCAGAGGAATCGCCAGCATGTTCTACCCTTCT |
| | | | ATC |
| 2406 | HC CDR1 | 172 | GGCATCGATCTGAGCAGCTCTACA |
| | HC CDR2 | 173 | ATTGCCAGCAGCGGCTCTGCC |
| | HC CDR3 | 174 | GTTAGACAGGTGCCCGGCTACAACGACAACAGA |
| | | | TATGTG |

Light chain CDRs

| | | | |
|---|---|---|---|
| 190K12 | LC CDR1 | 138 | GAGGGGATCTACAACAGA |
| | LC CDR2 | 139 | GGAGTGATC |
| | LC CDR3 | 140 | CAGCAGTACTGGTCGATTCCGTGGACT |
| | | 141 | CAGCAGTACTGGTCTATCCCCTGGACC |
| 366F5 | LC CDR1 | 288 | CAGTCGCTGCTGAACTCGGGGAACCAGAAGAAT |
| | | | GAT |
| | LC CDR2 | 150 | GGAGCATCA |
| | LC CDR3 | 151 | CAGAACGACCACTCCTATCCGCTGACT |
| 23P11 | LC CDR1 | 152 | GAGGACATCCACGGCTAT |
| | LC CDR2 | 153 | TACGCCTCT |
| | LC CDR3 | 163 | CTCGGAGATGGCAGCACCAGCGGCATCACA |
| 2406 | LC CDR1 | 164 | GAGAGCGTGTACAACGCCAACAGC |
| | LC CDR2 | 165 | GATGCCTCT |
| | LC CDR3 | 175 | GCCGGCTACAAAGGCAATGGCGACGCCGCT |

TABLE 6
Amino acid sequences of humanized mAb variable regions

| | | | SEQ ID | |
|---|---|---|---|---|
| Parent mAb | IgG chain | Variant | NO | Amino acid sequence of variable region |
| 190K12 | HC | H4 | 49 | QVQLVQSGAEVKKPGASVKVSCKASGYTFTTYNMHWV |
| | | | | RQAPGQGLEWMGAIFPGNGHISYAQKFQGRVTMTVDT |
| | | | | STSTVYMELSSLRSEDTAVYYCARGDYDESYFDYWGQ |
| | | | | GTLVTVSS |
| | LC | L1 | 50 | DIQMTQSPSSLSASVGDRVTITCKASEGIYNRLAWYQ |
| | | | | QKPGKAPKLLLYGVISLETGVPSRFSGSGSGTDYTLT |
| | | | | ISSLQPEDFATYYCQQYWSIPWTFGGGTKVEIK |
| | | L1-C80 | 51 | DIQMTQSPSSLSASVGDRVTITCKASEGIYNRLAWYQ |
| | | | | QKPGKAPKLLLYGVISLETGVPSRFSGSGSGTDYTLT |
| | | | | ISSLQCEDVATYYCQQYWSIPWTFGGGTKVEIK |
| 366F5 | HC | H2 | 52 | QVQLQQSGAELVKPGASVKISCKASGYAFRSYWVNWV |
| | | | | KQRPGKGLEWIGQIFPGDGDINYSGSFKGKATLTADK |
| | | | | SSSTAYMQLSSLTSEDSAVYFCARMDFYSWFPYWGQG |
| | | | | TLVTVSA |
| | LC | L1 | 53 | DIVMTQSPDSLAVSLGERATINCKSSQSLLNSGNQKN |
| | | | | DLAWYQQKPGQPPKLLIYGASTRESGVPDRFSGSGSG |
| | | | | TDFTLTISSLQAEDVAVYYCQNDHSYPLTFGQGTKLE |
| | | | | IK |
| | | L1-C80 | 54 | DIVMTQSPDSLAVSLGERATINCKSSQSLLNSGNQKN |
| | | | | DLAWYQQKPGQPPKLLIYGASTRESGVPDRFSGSGSG |
| | | | | TDFTLTISSLQCEDVAVYYCQNDHSYPLTFGQGTKLE |
| | | | | IK |
| 23P11 | HC | H1 | 55 | QELKESGGGLVTPGGTLTLTCTASGFSLSNYYINWVR |
| | | | | QAPGKGLEYIGFIPMYGTTHYANWAKGRFTISRTSTT |
| | | | | VDLKMASLTASDTATYFCARGIASMFYPSIWGPGTLV |
| | | | | TVSS |
| | LC | L1 | 56 | DVVMTQSPSSLSASVGDRVTITCQASEDIHGYLAWYQ |
| | | | | QKPGKAPKLLIYYASTLASGVPSRFSGSGSGTDFTLT |
| | | | | ISSLQPEDFATYYCLGDGSTSGITFGQGTKVEIK |
| | | L1-C80 | 57 | DVVMTQSPSSLSASVGDRVTITCQASEDIHGYLAWYQ |
| | | | | QKPGKAPKLLIYYASTLASGVPSRFSGSGSGTDFTLT |
| | | | | ISSLQCEDVATYYCLGDGSTSGITFGQGTKVEIK |
| 2406 | HC | H1 | 58 | EVQLVESGGGLIQPGGSLRLSCAASGIDLSSSTMAWV |
| | | | | RQAPGKGLEWIGIIASSGSAYYAGWAKGRFTISRDNS |
| | | | | KNTLYLQMNSLRAEDTAVYYCVRQVPGYNDNRYVWGQ |
| | | | | GTLVTVSS |
| | LC | L1 | 59 | AIVMTQSPSSLSASVGDRVTITCQASESVYNANSCSW |
| | | | | YQQKPGKAPKLLIYDASDLASGVPSRFSGSGSGTDFT |
| | | | | LTISSLQPEDFATYYCAGYKGNGDAAFGQGTKVEIK |
| | | L1-C80 | 60 | AIVMTQSPSSLSASVGDRVTITCQASESVYNANSCSW |
| | | | | YQQKPGKAPKLLIYDASDLASGVPSRFSGSGSGTDET |
| | | | | LTISSLQCEDVATYYCAGYKGNGDAAFGQGTKVEIK |

TABLE 7
Nucleic acid sequences encoding mAB variable regions

| | | | SEQ ID | Nucleic acid sequence encoding |
|---|---|---|---|---|
| Parent mAb | IgG chain | Variant | NO | variable region |
| 190K12 | HC | H4 | 178 | CAAGTGCAGCTTGTGCAGTCCGGAGCTGAAGTCA |
| | | | | AGAAGCCTGGTGCCAGCGTGAAAGTGTCCTGCAA |
| | | | | GGCCTCCGGATACACCTTCACCACGTACAACATG |
| | | | | CATTGGGTCAGACAGGCACCGGGACAGGGTCTGG |
| | | | | AATGGATGGGGGCCATTTTTCCCGGAAACGGCCA |
| | | | | CATCTCGTACGCCCAAAAGTTCCAGGGCCGCGTG |
| | | | | ACCATGACTGTGGACACCTCCACCTCCACCGTGT |
| | | | | ACATGGAGCTCTCAAGCCTGAGGTCCGAGGATAC |
| | | | | TGCCGTGTACTATTGTGCGCGGGGGGATTACGAC |
| | | | | GAAAGCTACTTCGACTACTGGGGCCAGGGCACTC |
| | | | | TGGTCACCGTGTCGTCA |
| | LC | L1 | 179 | GACATTCAGATGACCCAATCCCCCTCATCACTGT |
| | | | | CGGCTTCCGTGGGCGACCGCGTGACCATCACCTG |
| | | | | TAAAGCCTCCGAGGGGATCTACAACAGACTCGCC |
| | | | | TGGTATCAGCAGAAGCCAGGAAAGGCCCCTAAGC |
| | | | | TCCTTCTGTACGGAGTGATCTCCCTGGAAACTGG |
| | | | | AGTGCCGAGCCGGTTTAGCGGCAGCGGTTCCGGA |
| | | | | ACTGACTACACGTTGACCATCTCCTCGCTGCAAC |
| | | | | CCGAAGATTTCGCGACCTACTACTGCCAGCAGTA |
| | | | | CTGGTCGATTCCGTGGACTTTCGGTGGCGGGACC |
| | | | | AAGGTCGAGATCAAA |
| | | L1-C80 | 180 | GATATTCAGATGACACAGAGCCCTAGCAGCCTGA |
| | | | | GCGCCTCTGTGGGCGATAGAGTGACAATCACATG |
| | | | | CAAGGCCAGCGAGGGCATCTACAACAGACTGGCC |
| | | | | TGGTATCAGCAGAAGCCCGGAAAGGCTCCTAAGC |
| | | | | TGCTGCTGTACGGCGTGATCTCTCTGGAAACAGG |
| | | | | CGTGCCAAGCAGATTTTCTGGCAGCGGCTCTGGC |
| | | | | ACCGACTACACCCTGACAATTTCTAGCCTGCAGT |
| | | | | GCGAGGACGTGGCCACCTACTACTGCCAGCAGTA |
| | | | | CTGGTCTATCCCCTGGACCTTTGGCGGAGGCACC |
| | | | | AAGGTGGAAATCAAG |
| 366F5 | HC | H2 | 181 | CAAGTGCAGCTGGTGCAGTCTGGTGCCGAGGTCA |
| | | | | AGAAGCCAGGCTCCAGCGTGAAAGTGTCCTGCAA |
| | | | | GGCCTCGGGATACGCATTCCGGTCGTACTGGATC |
| | | | | TCCTGGGTCCGCCAGGCCCCCGGACAAGGCCTTG |
| | | | | AATGGATGGGGCAGATCTTTCCTGGGGACGGCGA |
| | | | | CATTAACTACGCGCAGAAGTTCCAGGGCAGAGTG |
| | | | | ACCATCACTGCCGATAAGAGCACCTCCACTGCGT |
| | | | | ACATGGAACTGAGCTCCCTGAGGTCCGAGGATAC |
| | | | | CGCCGTGTACTATTGCGCTCGGATGGACTTCTAC |
| | | | | TCCTGGTTCCCGTACTGGGGACAGGGTACCCTCG |
| | | | | TCACCGTGTCATCA |
| | LC | L1 | 182 | GACATTGTGATGACCCAGTCCCCCGATTCCCTTG |
| | | | | CCGTGTCCCTCGGTGAACGGGCCACTATCAACTG |
| | | | | CAAATCCAGCCAGTCGCTGCTGAACTCGGGGAAC |
| | | | | CAGAAGAATGATCTGGCCTGGTACCAGCAGAAGC |
| | | | | CTGGCCAGCCCCCAAAGCTGCTCATCTACGGAGC |
| | | | | ATCAACTCGCGAATCCGGAGTGCCGGACAGATTT |
| | | | | TCCGGGAGCGGATCAGGCACCGACTTCACCTTGA |
| | | | | CCATTTCGAGCCTGCAAGCGGAGGACGTCGCTGT |
| | | | | GTACTACTGTCAGAACGACCACTCCTATCCGCTG |
| | | | | ACTTTCGGACAAGGCACCAAGCTGGAGATCAAA |
| | | L1-C80 | 183 | GACATTGTGATGACCCAGTCCCCCGATTCCCTTG |
| | | | | CCGTGTCCCTCGGTGAACGGGCCACTATCAACTG |
| | | | | CAAATCCAGCCAGTCGCTGCTGAACTCGGGGAAC |
| | | | | CAGAAGAATGATCTGGCCTGGTACCAGCAGAAGC |
| | | | | CTGGCCAGCCCCCAAAGCTGCTCATCTACGGAGC |
| | | | | ATCAACTCGCGAATCCGGAGTGCCGGACAGATTT |
| | | | | TCCGGGAGCGGATCAGGCACCGACTTCACCTTGA |
| | | | | CCATTTCGAGCCTGCAATGCGAGGACGTCGCTGT |
| | | | | GTACTACTGTCAGAACGACCACTCCTATCCGCTG |
| | | | | ACTTTCGGACAAGGCACCAAGCTGGAGATCAAA |
| 23P11 | HC | \|H1 | 184 | GAAGTGCAGCTGGTTGAATCTGGCGGAGGACTGA |
| | | | | TTCAGCCTGGCGGCTCTCTGAGACTGTCTTGTGC |
| | | | | CGCCTCTGGCTTCAGCCTGAGCAACTACTACATC |
| | | | | AACTGGGTCCGACAGGCCCCTGGCAAAGGACTGG |
| | | | | AATGGATCGGCTTCATCCCTATGTACGGCACCAC |
| | | | | ACACTACGCCAACTGGGCCAAGGGCAGATTCACC |
| | | | | ATCAGCCGGGACAACAGCAAGAACACCCTGTACC |
| | | | | TGCAGATGAACAGCCTGAGAGCCGAGGACACCGC |
| | | | | CGTGTACTATTGTGCCAGAGGAATCGCCAGCATG |
| | | | | TTCTACCCTTCTATCTGGGGCCAGGGCACCCTGG |
| | | | | TCACAGTTAGTTCT |
| | LC | L1 | 185 | GACGTTGTGATGACACAGAGCCCTAGCAGCCTGA |
| | | | | GCGCCTCTGTGGGCGATAGAGTGACAATCACCTG |
| | | | | TCAGGCCAGCGAGGACATCCACGGCTATCTGGCC |
| | | | | TGGTATCAGCAGAAGCCTGGAAAGGCCCCTAAGC |
| | | | | TGCTGATCTACTACGCCTCTACACTGGCCAGCGG |
| | | | | CGTGCCAAGCAGATTTTCTGGCTCTGGCAGCGGC |
| | | | | ACCGACTTCACCCTGACAATTTCTAGCCTCCAGC |
| | | | | CTGAGGACTTCGCCACCTACTATTGTCTCGGAGA |
| | | | | TGGCAGCACCAGCGGCATCACATTTGGCCAGGGC |
| | | | | ACCAAGGTGGAAATCAAA |
| | | L1-C80 | 186 | GACGTTGTGATGACACAGAGCCCTAGCAGCCTGA |
| | | | | GCGCCTCTGTGGGCGATAGAGTGACAATCACCTG |
| | | | | TCAGGCCAGCGAGGACATCCACGGCTATCTGGCC |
| | | | | TGGTATCAGCAGAAGCCTGGAAAGGCCCCTAAGC |
| | | | | TGCTGATCTACTACGCCTCTACACTGGCCAGCGG |
| | | | | CGTGCCAAGCAGATTTTCTGGCTCTGGCAGCGGC |
| | | | | ACCGACTTCACCCTGACAATTTCTAGCCTCCAGT |
| | | | | GCGAGGACGTGGCCACCTACTATTGTCTCGGAGA |
| | | | | TGGCAGCACCAGCGGCATCACATTTGGCCAGGGC |
| | | | | ACCAAGGTGGAAATCAAA |
| 2406 | HC | H1 | 187 | GAAGTGCAGCTGGTTGAATCTGGCGGAGGACTGA |
| | | | | TTCAGCCTGGCGGCTCTCTGAGACTGTCTTGTGC |
| | | | | CGCCTCTGGCATCGATCTGAGCAGCTCTACAATG |
| | | | | GCCTGGGTTCGACAGGCCCCTGGCAAAGGACTGG |
| | | | | AATGGATCGGCATCATTGCCAGCAGCGGCTCTGC |
| | | | | CTATTATGCCGGATGGGCCAAGGGCAGATTCACC |
| | | | | ATCAGCCGGGACAACAGCAAGAACACCCTGTACC |
| | | | | TGCAGATGAACAGCCTGAGAGCCGAGGACACCGC |
| | | | | CGTGTACTATTGTGTTAGACAGGTGCCCGGCTAC |
| | | | | AACGACAACAGATATGTGTGGGGCCAGGGCACCC |
| | | | | TGGTCACAGTTAGTTCT |
| | LC | L1 | 188 | GCTATTGTGATGACACAGAGCCCTAGCAGCCTGA |
| | | | | GCGCCTCTGTGGGCGATAGAGTGACAATCACCTG |
| | | | | TCAGGCCAGCGAGAGCGTGTACAACGCCAACAGC |
| | | | | TGTAGCTGGTATCAGCAGAAGCCTGGCAAGGCCC |
| | | | | CTAAGCTGCTGATCTACGATGCCTCTGATCTGGC |
| | | | | CAGCGGCGTGCCAAGCAGATTTTCTGGCTCTGGC |
| | | | | AGCGGCACCGACTTCACCCTGACAATTTCTAGCC |
| | | | | TCCAGCCTGAGGACTTCGCCACCTACTATTGTGC |
| | | | | CGGCTACAAAGGCAATGGCGACGCCGCTTTTGGC |
| | | | | CAGGGCACAAAGGTGGAAATCAAA |
| | | L1-C80 | 189 | GCTATTGTGATGACACAGAGCCCTAGCAGCCTGA |
| | | | | GCGCCTCTGTGGGCGATAGAGTGACAATCACCTG |
| | | | | TCAGGCCAGCGAGAGCGTGTACAACGCCAACAGC |
| | | | | TGTAGCTGGTATCAGCAGAAGCCTGGCAAGGCCC |
| | | | | CTAAGCTGCTGATCTACGATGCCTCTGATCTGGC |
| | | | | CAGCGGCGTGCCAAGCAGATTTTCTGGCTCTGGC |
| | | | | AGCGGCACCGACTTCACCCTGACAATTTCTAGCC |
| | | | | TCCAGTGCGAGGACGTGGCCACCTACTATTGTGC |
| | | | | CGGCTACAAAGGCAATGGCGACGCCGCTTTTGGC |
| | | | | CAGGGCACAAAGGTGGAAATCAAA |

TABLE 8
Amino acid sequences of full-length mAb Ig chains

| mAb | IgG chain | Variant | SEQ ID NO | Amino acid sequence |
|---|---|---|---|---|
| 190K12 | HC | H4 | 61 | QVQLVQSGAEVKKPGASVKVSCKASGYTFTTYN |
| | | | | MHWVRQAPGQGLEWMGAIFPGNGHISYAQKFQG |
| | | | | RVTMTVDTSTSTVYMELSSLRSEDTAVYYCARG |
| | | | | DYDESYFDYWGQGTLVTVSSASTKGPSVEPLAP |
| | | | | SSKSTSGGTAALGCLVKDYFPEPVTVSWNSGAL |
| | | | | TSGVHTFPAVLQSSGLYSLSSVVTVPSSSLGTQ |
| | | | | TYICNVNHKPSNTKVDKKVEPKSCDKTHTCPPC |
| | | | | PAPELLGGPSVFLFPPKPKDTLMISRTPEVTCV |
| | | | | VVDVSHEDPEVKENWYVDGVEVHNAKTKPREEQ |
| | | | | YNSTYRVVSVLTVLHQDWLNGKEYKCKVSNKAL |
| | | | | PAPIEKTISKAKGQPREPQVYTLPPSRDELTKN |
| | | | | QVSLTCLVKGFYPSDIAVEWESNGQPENNYKTT |
| | | | | PPVLDSDGSFFLYSKLTVDKSRWQQGNVFSCSV |
| | | | | MHEALHNHYTQKSLSLSPGK |
| 190K12 | HC | H4-A118C | 62 | QVQLVQSGAEVKKPGASVKVSCKASGYTFTTYN |
| | | | | MHWVRQAPGQGLEWMGAIFPGNGHISYAQKFQG |
| | | | | RVTMTVDTSTSTVYMELSSLRSEDTAVYYCARG |
| | | | | DYDESYFDYWGQGTLVTVSSCSTKGPSVFPLAP |
| | | | | SSKSTSGGTAALGCLVKDYFPEPVTVSWNSGAL |
| | | | | TSGVHTFPAVLQSSGLYSLSSVVTVPSSSLGTQ |
| | | | | TYICNVNHKPSNTKVDKKVEPKSCDKTHTCPPC |
| | | | | PAPELLGGPSVFLFPPKPKDTLMISRTPEVTCV |
| | | | | VVDVSHEDPEVKENWYVDGVEVHNAKIKPREEQ |
| | | | | YNSTYRVVSVLTVLHQDWLNGKEYKCKVSNKAL |
| | | | | PAPIEKTISKAKGQPREPQVYTLPPSRDELTKN |
| | | | | QVSLTCLVKGFYPSDIAVEWESNGQPENNYKTT |
| | | | | PPVLDSDGSFFLYSKLTVDKSRWQQGNVFSCSV |
| | | | | MHEALHNHYTQKSLSLSPGK |
| 190K12 | HC | H4-14AAS | 63 | QVQLVQSGAEVKKPGASVKVSCKASGYTFTTYN |
| | | | | MHWVRQAPGQGLEWMGAIFPGNGHISYAQKFQG |
| | | | | RVTMTVDTSTSTVYMELSSLRSEDTAVYYCARG |
| | | | | DYDESYFDYWGQGTLVTVSSASTKGPSVFPLAP |
| | | | | SSKSTSGGTAALGCLVKDYFPEPVTVSWNSGAL |
| | | | | TSGVHTFPAVLQSSGLYSLSSVVTVPSSSLGTQ |
| | | | | TYICNVNHKPSNTKVDKKVEPKSCDKTHTCPPC |
| | | | | PAPEAAGGSSVFLFPPKPKDTLMISRTPEVTCV |
| | | | | VVDVSQEDPEVQFNWYVDGVEVHNAKTKPREEQ |
| | | | | YNSTYRVVSVLTVLHQDWLNGKEYKCKVSNKAL |
| | | | | PAPIEKTISKAKGQPREPQVYTLPPSRDELTKN |
| | | | | QVSLTCLVKGFYPSDIAVEWESNGQPENNYKTT |
| | | | | PPVLDSDGSFFLYSKLTVDKSRWQQGNVFSCSV |
| | | | | MHEALHNHYTQKSLSLSPGK |
| 190K12 | HC | H4-14AAS- | 64 | QVQLVQSGAEVKKPGASVKVSCKASGYTFTTYN |
| | | A118C | | MHWVRQAPGQGLEWMGAIFPGNGHISYAQKFQG |
| | | | | RVTMTVDTSTSTVYMELSSLRSEDTAVYYCARG |
| | | | | DYDESYFDYWGQGTLVTVSSCSTKGPSVFPLAP |
| | | | | SSKSTSGGTAALGCLVKDYFPEPVTVSWNSGAL |
| | | | | TSGVHTFPAVLQSSGLYSLSSVVTVPSSSLGTQ |
| | | | | TYICNVNHKPSNTKVDKKVEPKSCDKTHTCPPC |
| | | | | PAPEAAGGSSVFLFPPKPKDTLMISRTPEVTCV |
| | | | | VVDVSQEDPEVQFNWYVDGVEVHNAKTKPREEQ |
| | | | | YNSTYRVVSVLTVLHQDWLNGKEYKCKVSNKAL |
| | | | | PAPIEKTISKAKGQPREPQVYTLPPSRDELTKN |
| | | | | QVSLTCLVKGFYPSDIAVEWESNGQPENNYKIT |
| | | | | PPVLDSDGSFFLYSKLTVDKSRWQQGNVFSCSV |
| | | | | MHEALHNHYTQKSLSLSPGK |
| 190K12 | HC | H4 (-K447) | 111 | QVQLVQSGAEVKKPGASVKVSCKASGYTFTTYN |
| | | | | MHWVRQAPGQGLEWMGAIFPGNGHISYAQKFQG |
| | | | | RVTMTVDTSTSTVYMELSSLRSEDTAVYYCARG |
| | | | | DYDESYFDYWGQGTLVTVSSASTKGPSVFPLAP |
| | | | | SSKSTSGGTAALGCLVKDYFPEPVTVSWNSGAL |
| | | | | TSGVHTFPAVLQSSGLYSLSSVVTVPSSSLGTQ |
| | | | | TYICNVNHKPSNTKVDKKVEPKSCDKTHTCPPC |
| | | | | PAPELLGGPSVFLFPPKPKDTLMISRTPEVTCV |
| | | | | VVDVSHEDPEVKFNWYVDGVEVHNAKTKPREEQ |
| | | | | YNSTYRVVSVLTVLHQDWLNGKEYKCKVSNKAL |
| | | | | PAPIEKTISKAKGQPREPQVYTLPPSRDELTKN |
| | | | | QVSLTCLVKGFYPSDIAVEWESNGQPENNYKTT |
| | | | | PPVLDSDGSFFLYSKLTVDKSRWQQGNVFSCSV |
| | | | | MHEALHNHYTQKSLSLSPG |
| 190K12 | HC | H4-A118C | 112 | QVQLVQSGAEVKKPGASVKVSCKASGYTFTTYN |
| | | (-K447) | | MHWVRQAPGQGLEWMGAIFPGNGHISYAQKFQG |
| | | | | RVTMTVDTSTSTVYMELSSLRSEDTAVYYCARG |
| | | | | DYDESYFDYWGQGTLVTVSSCSTKGPSVFPLAP |
| | | | | SSKSTSGGTAALGCLVKDYFPEPVTVSWNSGAL |
| | | | | TSGVHTFPAVLQSSGLYSLSSVVTVPSSSLGTQ |
| | | | | TYICNVNHKPSNTKVDKKVEPKSCDKTHTCPPC |
| | | | | PAPELLGGPSVFLFPPKPKDTLMISRTPEVTCV |
| | | | | VVDVSHEDPEVKFNWYVDGVEVHNAKTKPREEQ |
| | | | | YNSTYRVVSVLTVLHQDWLNGKEYKCKVSNKAL |
| | | | | PAPIEKTISKAKGQPREPQVYTLPPSRDELTKN |
| | | | | QVSLTCLVKGFYPSDIAVEWESNGQPENNYKTT |
| | | | | PPVLDSDGSFFLYSKLTVDKSRWQQGNVFSCSV |
| | | | | MHEALHNHYTQKSLSLSPG |
| 190K12 | HC | H4-14AAS | 113 | QVQLVQSGAEVKKPGASVKVSCKASGYTFTTYN |
| | | (-K447) | | MHWVRQAPGQGLEWMGAIFPGNGHISYAQKFQG |
| | | | | RVTMTVDTSTSTVYMELSSLRSEDTAVYYCARG |
| | | | | DYDESYFDYWGQGTLVTVSSASTKGPSVFPLAP |
| | | | | SSKSTSGGTAALGCLVKDYFPEPVTVSWNSGAL |
| | | | | TSGVHTFPAVLQSSGLYSLSSVVTVPSSSLGTQ |
| | | | | TYICNVNHKPSNTKVDKKVEPKSCDKTHTCPPC |
| | | | | PAPEAAGGSSVFLFPPKPKDTLMISRTPEVTCV |
| | | | | VVDVSQEDPEVQFNWYVDGVEVHNAKTKPREEQ |
| | | | | YNSTYRVVSVLTVLHQDWLNGKEYKCKVSNKAL |
| | | | | PAPIEKTISKAKGQPREPQVYTLPPSRDELTKN |
| | | | | QVSLTCLVKGFYPSDIAVEWESNGQPENNYKTT |
| | | | | PPVLDSDGSFFLYSKLTVDKSRWQQGNVFSCSV |
| | | | | MHEALHNHYTQKSLSLSPG |
| 190K12 | HC | H4-14AAS- | 114 | QVQLVQSGAEVKKPGASVKVSCKASGYTFTTYN |
| | | A118C | | MHWVRQAPGQGLEWMGAIFPGNGHISYAQKFQG |
| | | (-K447) | | RVTMTVDTSTSTVYMELSSLRSEDTAVYYCARG |
| | | | | DYDESYFDYWGQGTLVTVSSCSTKGPSVEPLAP |
| | | | | SSKSTSGGTAALGCLVKDYFPEPVTVSWNSGAL |
| | | | | TSGVHTFPAVLQSSGLYSLSSVVTVPSSSLGTQ |
| | | | | TYICNVNHKPSNTKVDKKVEPKSCDKTHTCPPC |
| | | | | PAPEAAGGSSVFLFPPKPKDTLMISRTPEVTCV |
| | | | | VVDVSQEDPEVQFNWYVDGVEVHNAKTKPREEQ |
| | | | | YNSTYRVVSVLTVLHQDWLNGKEYKCKVSNKAL |
| | | | | PAPIEKTISKAKGQPREPQVYTLPPSRDELTKN |
| | | | | QVSLTCLVKGFYPSDIAVEWESNGQPENNYKTT |
| | | | | PPVLDSDGSFFLYSKLTVDKSRWQQGNVFSCSV |
| | | | | MHEALHNHYTQKSLSLSPG |
| 190K12 | LC | L1 | 65 | DIQMTQSPSSLSASVGDRVTITCKASEGIYNRL |
| | | | | AWYQQKPGKAPKLLLYGVISLETGVPSRFSGSG |
| | | | | SGTDYTLTISSLQPEDFATYYCQQYWSIPWTFG |
| | | | | GGTKVEIKRTVAAPSVFIFPPSDEQLKSGTASV |
| | | | | VCLLNNFYPREAKVQWKVDNALQSGNSQESVTE |
| | | | | QDSKDSTYSLSSTLTLSKADYEKHKVYACEVTH |
| | | | | QGLSSPVTKSENRGEC |
| 190K12 | LC | L1-C80 | 66 | DIQMTQSPSSLSASVGDRVTITCKASEGIYNRL |
| | | | | AWYQQKPGKAPKLLLYGVISLETGVPSRFSGSG |
| | | | | SGTDYTLTISSLQCEDVATYYCQQYWSIPWTFG |
| | | | | GGTKVEIKRTVAAPSVFIFPPSDEQLKSGTASV |
| | | | | VCLLNNFYPREAKVQWKVDNALQSGNSQESVTE |
| | | | | QDSKDSTYSLSSTLTLSKADYEKHKVYACEVTH |
| | | | | QGLSSPVTKSENRGEC |
| 366F5 | HC | H2 | 67 | QVQLVQSGAEVKKPGSSVKVSCKASGYAFRSYW |
| | | | | ISWVRQAPGQGLEWMGQIFPGDGDINYAQKFQG |
| | | | | RVTITADKSTSTAYMELSSLRSEDTAVYYCARM |
| | | | | DFYSWFPYWGQGTLVTVSSASTKGPSVFPLAPS |
| | | | | SKSTSGGTAALGCLVKDYFPEPVTVSWNSGALT |
| | | | | SGVHTFPAVLQSSGLYSLSSVVTVPSSSLGTQT |
| | | | | YICNVNHKPSNTKVDKKVEPKSCDKTHTCPPCP |
| | | | | APELLGGPSVFLFPPKPKDTLMISRTPEVTCVV |
| | | | | VDVSHEDPEVKFNWYVDGVEVHNAKTKPREEQY |
| | | | | NSTYRVVSVLTVLHQDWLNGKEYKCKVSNKALP |
| | | | | APIEKTISKAKGQPREPQVYTLPPSRDELTKNQ |
| | | | | VSLTCLVKGFYPSDIAVEWESNGQPENNYKTTP |
| | | | | PVLDSDGSFFLYSKLTVDKSRWQQGNVFSCSVM |
| | | | | HEALHNHYTQKSLSLSPGK |
| 366F5 | HC | H2-A118C | 68 | QVQLVQSGAEVKKPGSSVKVSCKASGYAFRSYW |
| | | | | ISWVRQAPGQGLEWMGQIFPGDGDINYAQKFQG |
| | | | | RVTITADKSTSTAYMELSSLRSEDTAVYYCARM |
| | | | | DFYSWFPYWGQGTLVTVSSCSTKGPSVFPLAPS |
| | | | | SKSTSGGTAALGCLVKDYFPEPVTVSWNSGALT |
| | | | | SGVHTFPAVLQSSGLYSLSSVVTVPSSSLGTQT |
| | | | | YICNVNHKPSNTKVDKKVEPKSCDKTHTCPPCP |
| | | | | APELLGGPSVFLFPPKPKDTLMISRTPEVTCVV |
| | | | | VDVSHEDPEVKFNWYVDGVEVHNAKTKPREEQY |
| | | | | NSTYRVVSVLTVLHQDWLNGKEYKCKVSNKALP |
| | | | | APIEKTISKAKGQPREPQVYTLPPSRDELTKNQ |
| | | | | VSLTCLVKGFYPSDIAVEWESNGQPENNYKTTP |
| | | | | PVLDSDGSFFLYSKLTVDKSRWQQGNVFSCSVM |
| | | | | HEALHNHYTQKSLSLSPGK |
| 366F5 | HC | H2-14AAS | 69 | QVQLVQSGAEVKKPGSSVKVSCKASGYAFRSYW |
| | | | | ISWVRQAPGQGLEWMGQIFPGDGDINYAQKFQG |
| | | | | RVTITADKSTSTAYMELSSLRSEDTAVYYCARM |
| | | | | DFYSWFPYWGQGTLVTVSSASTKGPSVFPLAPS |
| | | | | SKSTSGGTAALGCLVKDYFPEPVTVSWNSGALT |
| | | | | SGVHTFPAVLQSSGLYSLSSVVTVPSSSLGTQT |
| | | | | YICNVNHKPSNTKVDKKVEPKSCDKTHTCPPCP |
| | | | | APEAAGGSSVFLFPPKPKDTLMISRTPEVTCVV |
| | | | | VDVSQEDPEVQFNWYVDGVEVHNAKTKPREEQY |
| | | | | NSTYRVVSVLTVLHQDWLNGKEYKCKVSNKALP |
| | | | | APIEKTISKAKGQPREPQVYTLPPSRDELTKNQ |
| | | | | VSLTCLVKGFYPSDIAVEWESNGQPENNYKTTP |
| | | | | PVLDSDGSFFLYSKLTVDKSRWQQGNVFSCSVM |
| | | | | HEALHNHYTQKSLSLSPGK |
| 366F5 | HC | H2-14AAS- | 70 | QVQLVQSGAEVKKPGASVKVSCKASGYTFTTYN |
| | | A118C | | MHWVRQAPGQGLEWMGAIFPGNGHISYAQKFQG |
| | | | | RVTMTVDTSTSTVYMELSSLRSEDTAVYYCARG |
| | | | | DYDESYFDYWGQGTLVTVSSCSTKGPSVFPLAP |
| | | | | SSKSTSGGTAALGCLVKDYFPEPVTVSWNSGAL |
| | | | | TSGVHTFPAVLQSSGLYSLSSVVTVPSSSLGTQ |
| | | | | TYICNVNHKPSNTKVDKKVEPKSCDKTHTCPPC |
| | | | | PAPEAAGGSSVFLFPPKPKDTLMISRTPEVTCV |
| | | | | VVDVSQEDPEVQFNWYVDGVEVHNAKTKPREEQ |
| | | | | YNSTYRVVSVLTVLHQDWLNGKEYKCKVSNKAL |
| | | | | PAPIEKTISKAKGQPREPQVYTLPPSRDELTKN |
| | | | | QVSLTCLVKGFYPSDIAVEWESNGQPENNYKTT |
| | | | | PPVLDSDGSFFLYSKLTVDKSRWQQGNVFSCSV |
| | | | | MHEALHNHYTQKSLSLSPGK |
| 366F5 | HC | H2 (-K447) | 115 | QVQLVQSGAEVKKPGSSVKVSCKASGYAFRSYW |
| | | | | ISWVRQAPGQGLEWMGQIFPGDGDINYAQKFQG |
| | | | | RVTITADKSTSTAYMELSSLRSEDTAVYYCARM |
| | | | | DFYSWFPYWGQGTLVTVSSASTKGPSVFPLAPS |
| | | | | SKSTSGGTAALGCLVKDYFPEPVTVSWNSGALT |
| | | | | SGVHTFPAVLQSSGLYSLSSVVTVPSSSLGTQT |
| | | | | YICNVNHKPSNTKVDKKVEPKSCDKTHTCPPCP |
| | | | | APELLGGPSVFLFPPKPKDTLMISRTPEVTCVV |
| | | | | VDVSHEDPEVKFNWYVDGVEVHNAKTKPREEQY |
| | | | | NSTYRVVSVLTVLHQDWLNGKEYKCKVSNKALP |
| | | | | APIEKTISKAKGQPREPQVYTLPPSRDELTKNQ |
| | | | | VSLTCLVKGFYPSDIAVEWESNGQPENNYKTTP |
| | | | | PVLDSDGSFFLYSKLTVDKSRWQQGNVFSCSVM |
| | | | | HEALHNHYTQKSLSLSPG |
| 366F5 | HC | H2-A118C | 116 | QVQLVQSGAEVKKPGSSVKVSCKASGYAFRSYW |
| | | (-K447) | | ISWVRQAPGQGLEWMGQIFPGDGDINYAQKFQG |
| | | | | RVTITADKSTSTAYMELSSLRSEDTAVYYCARM |
| | | | | DFYSWFPYWGQGTLVTVSSCSTKGPSVFPLAPS |
| | | | | SKSTSGGTAALGCLVKDYFPEPVTVSWNSGALT |
| | | | | SGVHTFPAVLQSSGLYSLSSVVTVPSSSLGTQT |
| | | | | YICNVNHKPSNTKVDKKVEPKSCDKTHTCPPCP |
| | | | | APELLGGPSVFLFPPKPKDTLMISRTPEVTCVV |
| | | | | VDVSHEDPEVKFNWYVDGVEVHNAKTKPREEQY |
| | | | | NSTYRVVSVLTVLHQDWLNGKEYKCKVSNKALP |
| | | | | APIEKTISKAKGQPREPQVYTLPPSRDELTKNQ |
| | | | | VSLTCLVKGFYPSDIAVEWESNGQPENNYKTTP |
| | | | | PVLDSDGSFFLYSKLTVDKSRWQQGNVFSCSVM |
| | | | | HEALHNHYTQKSLSLSPG |
| 366F5 | HC | H2-14AAS | 117 | QVQLVQSGAEVKKPGSSVKVSCKASGYAFRSYW |
| | | (-K447) | | ISWVRQAPGQGLEWMGQIFPGDGDINYAQKFQG |
| | | | | RVTITADKSTSTAYMELSSLRSEDTAVYYCARM |
| | | | | DFYSWFPYWGQGTLVTVSSASTKGPSVFPLAPS |
| | | | | SKSTSGGTAALGCLVKDYFPEPVTVSWNSGALT |
| | | | | SGVHTFPAVLQSSGLYSLSSVVTVPSSSLGTQT |
| | | | | YICNVNHKPSNTKVDKKVEPKSCDKTHTCPPCP |
| | | | | APEAAGGSSVFLFPPKPKDTLMISRTPEVTCVV |
| | | | | VDVSQEDPEVQFNWYVDGVEVHNAKTKPREEQY |
| | | | | NSTYRVVSVLTVLHQDWLNGKEYKCKVSNKALP |
| | | | | APIEKTISKAKGQPREPQVYTLPPSRDELTKNQ |
| | | | | VSLTCLVKGFYPSDIAVEWESNGQPENNYKTTP |
| | | | | PVLDSDGSFFLYSKLTVDKSRWQQGNVFSCSVM |
| | | | | HEALHNHYTQKSLSLSPG |
| 366F5 | HC | H2-14AAS- | 118 | QVQLVQSGAEVKKPGASVKVSCKASGYTFTTYN |
| | | A118C | | MHWVRQAPGQGLEWMGAIFPGNGHISYAQKFQG |
| | | (-K447) | | RVTMTVDTSTSTVYMELSSLRSEDTAVYYCARG |
| | | | | DYDESYFDYWGQGTLVTVSSCSTKGPSVFPLAP |
| | | | | SSKSTSGGTAALGCLVKDYFPEPVTVSWNSGAL |
| | | | | TSGVHTFPAVLQSSGLYSLSSVVTVPSSSLGTQ |
| | | | | TYICNVNHKPSNTKVDKKVEPKSCDKTHTCPPC |
| | | | | PAPEAAGGSSVFLFPPKPKDTLMISRTPEVTCV |
| | | | | VVDVSQEDPEVQFNWYVDGVEVHNAKTKPREEQ |
| | | | | YNSTYRVVSVLTVLHQDWLNGKEYKCKVSNKAL |
| | | | | PAPIEKTISKAKGQPREPQVYTLPPSRDELTKN |
| | | | | QVSLTCLVKGFYPSDIAVEWESNGQPENNYKTT |
| | | | | PPVLDSDGSFFLYSKLTVDKSRWQQGNVFSCSV |
| | | | | MHEALHNHYTQKSLSLSPG |
| 366F5 | LC | L1 | 71 | DIVMTQSPDSLAVSLGERATINCKSSQSLLNSG |
| | | | | NQKNDLAWYQQKPGQPPKLLIYGASTRESGVPD |
| | | | | RFSGSGSGTDFTLTISSLQAEDVAVYYCQNDHS |
| | | | | YPLTFGQGTKLEIKRTVAAPSVFIFPPSDEQLK |
| | | | | SGTASVVCLLNNFYPREAKVQWKVDNALQSGNS |
| | | | | QESVTEQDSKDSTYSLSSTLTLSKADYEKHKVY |
| | | | | ACEVTHQGLSSPVTKSENRGEC |
| 366F5 | LC | L1-C80 | 72 | DIVMTQSPDSLAVSLGERATINCKSSQSLLNSG |
| | | | | NQKNDLAWYQQKPGQPPKLLIYGASTRESGVPD |
| | | | | RFSGSGSGTDFTLTISSLQCEDVAVYYCQNDHS |
| | | | | YPLTFGQGTKLEIKRTVAAPSVFIFPPSDEQLK |
| | | | | SGTASVVCLLNNFYPREAKVQWKVDNALQSGNS |
| | | | | QESVTEQDSKDSTYSLSSTLTLSKADYEKHKVY |
| | | | | ACEVTHQGLSSPVTKSENRGEC |
| 23P11 | HC | H1 | 73 | EVQLVESGGGLIQPGGSLRLSCAASGFSLSNYY |
| | | | | INWVRQAPGKGLEWIGFIPMYGTTHYANWAKGR |
| | | | | FTISRDNSKNTLYLQMNSLRAEDTAVYYCARGI |
| | | | | ASMFYPSIWGQGTLVTVSSASTKGPSVFPLAPS |
| | | | | SKSTSGGTAALGCLVKDYFPEPVTVSWNSGALT |
| | | | | SGVHTFPAVLQSSGLYSLSSVVTVPSSSLGTQT |
| | | | | YICNVNHKPSNTKVDKKVEPKSCDKTHTCPPCP |
| | | | | APELLGGPSVFLFPPKPKDTLMISRTPEVTCVV |
| | | | | VDVSHEDPEVKFNWYVDGVEVHNAKTKPREEQY |
| | | | | NSTYRVVSVLTVLHQDWLNGKEYKCKVSNKALP |
| | | | | APIEKTISKAKGQPREPQVYTLPPSRDELTKNQ |
| | | | | VSLTCLVKGFYPSDIAVEWESNGQPENNYKTTP |
| | | | | PVLDSDGSFFLYSKLTVDKSRWQQGNVFSCSVM |
| | | | | HEALHNHYTQKSLSLSPGK |
| 23P11 | HC | H1-A118C | 74 | EVQLVESGGGLIQPGGSLRLSCAASGFSLSNYY |
| | | | | INWVRQAPGKGLEWIGFIPMYGTTHYANWAKGR |
| | | | | FTISRDNSKNTLYLQMNSLRAEDTAVYYCARGI |
| | | | | ASMFYPSIWGQGTLVTVSSCSTKGPSVFPLAPS |
| | | | | SKSTSGGTAALGCLVKDYFPEPVTVSWNSGALT |
| | | | | SGVHTFPAVLQSSGLYSLSSVVTVPSSSLGTQT |
| | | | | YICNVNHKPSNTKVDKKVEPKSCDKTHTCPPCP |
| | | | | APELLGGPSVFLFPPKPKDTLMISRTPEVTCVV |
| | | | | VDVSHEDPEVKFNWYVDGVEVHNAKTKPREEQY |
| | | | | NSTYRVVSVLTVLHQDWLNGKEYKCKVSNKALP |
| | | | | APIEKTISKAKGQPREPQVYTLPPSRDELTKNQ |
| | | | | VSLTCLVKGFYPSDIAVEWESNGQPENNYKTTP |
| | | | | PVLDSDGSFFLYSKLTVDKSRWQQGNVFSCSVM |
| | | | | HEALHNHYTQKSLSLSPGK |
| 23P11 | HC | H1-14AAS | 75 | EVQLVESGGGLIQPGGSLRLSCAASGFSLSNYY |
| | | | | INWVRQAPGKGLEWIGFIPMYGTTHYANWAKGR |
| | | | | FTISRDNSKNTLYLQMNSLRAEDTAVYYCARGI |
| | | | | ASMFYPSIWGQGTLVTVSSASTKGPSVFPLAPS |
| | | | | SKSTSGGTAALGCLVKDYFPEPVTVSWNSGALT |
| | | | | SGVHTFPAVLQSSGLYSLSSVVTVPSSSLGTQT |
| | | | | YICNVNHKPSNTKVDKKVEPKSCDKTHTCPPCP |
| | | | | APEAAGGSSVFLFPPKPKDTLMISRTPEVTCVV |
| | | | | VDVSQEDPEVQFNWYVDGVEVHNAKTKPREEQY |
| | | | | NSTYRVVSVLTVLHQDWLNGKEYKCKVSNKALP |
| | | | | APIEKTISKAKGQPREPQVYTLPPSRDELTKNQ |
| | | | | VSLTCLVKGFYPSDIAVEWESNGQPENNYKTTP |
| | | | | PVLDSDGSFFLYSKLTVDKSRWQQGNVFSCSVM |
| | | | | HEALHNHYTQKSLSLSPGK |
| 23P11 | HC | H1-14AAS- | 76 | EVQLVESGGGLIQPGGSLRLSCAASGESLSNYY |
| | | A118C | | INWVRQAPGKGLEWIGFIPMYGTTHYANWAKGR |
| | | | | FTISRDNSKNTLYLQMNSLRAEDTAVYYCARGI |
| | | | | ASMFYPSIWGQGTLVTVSSCSTKGPSVFPLAPS |
| | | | | SKSTSGGTAALGCLVKDYFPEPVTVSWNSGALT |
| | | | | SGVHTFPAVLQSSGLYSLSSVVTVPSSSLGTQT |
| | | | | YICNVNHKPSNTKVDKKVEPKSCDKTHTCPPCP |
| | | | | APEAAGGSSVFLFPPKPKDTLMISRTPEVTCVV |
| | | | | VDVSQEDPEVQFNWYVDGVEVHNAKTKPREEQY |
| | | | | NSTYRVVSVLTVLHQDWLNGKEYKCKVSNKALP |
| | | | | APIEKTISKAKGQPREPQVYTLPPSRDELTKNQ |
| | | | | VSLTCLVKGFYPSDIAVEWESNGQPENNYKTTP |
| | | | | PVLDSDGSFFLYSKLTVDKSRWQQGNVFSCSVM |
| | | | | HEALHNHYTQKSLSLSPGK |
| 23P11 | HC | H1 (-K447) | 119 | EVQLVESGGGLIQPGGSLRLSCAASGFSLSNYY |
| | | | | INWVRQAPGKGLEWIGFIPMYGTTHYANWAKGR |
| | | | | FTISRDNSKNTLYLQMNSLRAEDTAVYYCARGI |
| | | | | ASMFYPSIWGQGTLVTVSSASTKGPSVFPLAPS |
| | | | | SKSTSGGTAALGCLVKDYFPEPVTVSWNSGALT |
| | | | | SGVHTFPAVLQSSGLYSLSSVVTVPSSSLGTQT |
| | | | | YICNVNHKPSNTKVDKKVEPKSCDKTHTCPPCP |
| | | | | APELLGGPSVFLFPPKPKDTLMISRTPEVTCVV |
| | | | | VDVSHEDPEVKFNWYVDGVEVHNAKTKPREEQY |
| | | | | NSTYRVVSVLTVLHQDWLNGKEYKCKVSNKALP |
| | | | | APIEKTISKAKGQPREPQVYTLPPSRDELTKNQ |
| | | | | VSLTCLVKGFYPSDIAVEWESNGQPENNYKTTP |
| | | | | PVLDSDGSFFLYSKLTVDKSRWQQGNVFSCSVM |
| | | | | HEALHNHYTQKSLSLSPG |
| 23P11 | HC | H1-A118C | \|120 | EVQLVESGGGLIQPGGSLRLSCAASGFSLSNYY |
| | | (-K447) | | INWVRQAPGKGLEWIGFIPMYGTTHYANWAKGR |
| | | | | FTISRDNSKNTLYLQMNSLRAEDTAVYYCARGI |
| | | | | ASMFYPSIWGQGTLVTVSSCSTKGPSVFPLAPS |
| | | | | SKSTSGGTAALGCLVKDYFPEPVTVSWNSGALT |
| | | | | SGVHTFPAVLQSSGLYSLSSVVTVPSSSLGTQT |
| | | | | YICNVNHKPSNTKVDKKVEPKSCDKTHTCPPCP |
| | | | | APELLGGPSVFLFPPKPKDTLMISRTPEVTCVV |
| | | | | VDVSHEDPEVKFNWYVDGVEVHNAKTKPREEQY |
| | | | | NSTYRVVSVLTVLHQDWLNGKEYKCKVSNKALP |
| | | | | APIEKTISKAKGQPREPQVYTLPPSRDELTKNQ |
| | | | | VSLTCLVKGFYPSDIAVEWESNGQPENNYKTTP |
| | | | | PVLDSDGSFFLYSKLTVDKSRWQQGNVFSCSVM |
| | | | | HEALHNHYTQKSLSLSPGK |
| 23P11 | HC | H1-14AAS | 121 | EVQLVESGGGLIQPGGSLRLSCAASGFSLSNYY |
| | | (-K447) | | INWVRQAPGKGLEWIGFIPMYGTTHYANWAKGR |
| | | | | FTISRDNSKNTLYLQMNSLRAEDTAVYYCARGI |
| | | | | ASMFYPSIWGQGTLVTVSSASTKGPSVFPLAPS |
| | | | | SKSTSGGTAALGCLVKDYFPEPVTVSWNSGALT |
| | | | | SGVHTFPAVLQSSGLYSLSSVVTVPSSSLGTQT |
| | | | | YICNVNHKPSNTKVDKKVEPKSCDKTHTCPPCP |
| | | | | APEAAGGSSVFLFPPKPKDTLMISRTPEVTCVV |
| | | | | VDVSQEDPEVQFNWYVDGVEVHNAKTKPREEQY |
| | | | | NSTYRVVSVLTVLHQDWLNGKEYKCKVSNKALP |
| | | | | APIEKTISKAKGQPREPQVYTLPPSRDELTKNQ |
| | | | | VSLTCLVKGFYPSDIAVEWESNGQPENNYKTTP |
| | | | | PVLDSDGSFFLYSKLTVDKSRWQQGNVFSCSVM |
| | | | | HEALHNHYTQKSLSLSPG |
| 23P11 | HC | H1-14AAS- | 122 | EVQLVESGGGLIQPGGSLRLSCAASGFSLSNYY |
| | | A118C | | INWVRQAPGKGLEWIGFIPMYGTTHYANWAKGR |
| | | (-K447) | | FTISRDNSKNTLYLQMNSLRAEDTAVYYCARGI |
| | | | | ASMFYPSIWGQGTLVTVSSCSTKGPSVFPLAPS |
| | | | | SKSTSGGTAALGCLVKDYFPEPVTVSWNSGALT |
| | | | | SGVHTFPAVLQSSGLYSLSSVVTVPSSSLGTQT |
| | | | | YICNVNHKPSNTKVDKKVEPKSCDKTHTCPPCP |
| | | | | APEAAGGSSVFLFPPKPKDTLMISRTPEVTCVV |
| | | | | VDVSQEDPEVQFNWYVDGVEVHNAKTKPREEQY |
| | | | | NSTYRVVSVLTVLHQDWLNGKEYKCKVSNKALP |
| | | | | APIEKTISKAKGQPREPQVYTLPPSRDELTKNQ |
| | | | | VSLTCLVKGFYPSDIAVEWESNGQPENNYKTTP |
| | | | | PVLDSDGSFFLYSKLTVDKSRWQQGNVFSCSVM |
| | | | | HEALHNHYTQKSLSLSPG |
| 23P11 | LC | L1 | 77 | DVVMTQSPSSLSASVGDRVTITCQASEDIHGYL |
| | | | | AWYQQKPGKAPKLLIYYASTLASGVPSRFSGSG |
| | | | | SGTDFTLTISSLQPEDFATYYCLGDGSTSGITF |
| | | | | GQGTKVEIKRTVAAPSVFIFPPSDEQLKSGTAS |
| | | | | VVCLLNNFYPREAKVQWKVDNALQSGNSQESVT |
| | | | | EQDSKDSTYSLSSTLTLSKADYEKHKVYACEVT |
| | | | | HQGLSSPVTKSENRGEC |
| 23P11 | LC | L1-C80 | 78 | DVVMTQSPSSLSASVGDRVTITCQASEDIHGYL |
| | | | | AWYQQKPGKAPKLLIYYASTLASGVPSRFSGSG |
| | | | | SGTDFTLTISSLQCEDVATYYCLGDGSTSGITF |
| | | | | GQGTKVEIKRTVAAPSVFIFPPSDEQLKSGTAS |
| | | | | VVCLLNNFYPREAKVQWKVDNALQSGNSQESVT |
| | | | | EQDSKDSTYSLSSTLTLSKADYEKHKVYACEVT |
| | | | | HQGLSSPVTKSENRGEC |
| 2406 | HC | H1 | 79 | EVQLVESGGGLIQPGGSLRLSCAASGIDLSSST |
| | | | | MAWVRQAPGKGLEWIGIIASSGSAYYAGWAKGR |
| | | | | FTISRDNSKNTLYLQMNSLRAEDTAVYYCVRQV |
| | | | | PGYNDNRYVWGQGTLVTVSSASTKGPSVEPLAP |
| | | | | SSKSTSGGTAALGCLVKDYFPEPVTVSWNSGAL |
| | | | | TSGVHTFPAVLQSSGLYSLSSVVTVPSSSLGTQ |
| | | | | TYICNVNHKPSNTKVDKKVEPKSCDKTHTCPPC |
| | | | | PAPELLGGPSVFLFPPKPKDTLMISRTPEVTCV |
| | | | | VVDVSHEDPEVKFNWYVDGVEVHNAKTKPREEQ |
| | | | | YNSTYRVVSVLTVLHQDWLNGKEYKCKVSNKAL |
| | | | | PAPIEKTISKAKGQPREPQVYTLPPSRDELTKN |
| | | | | QVSLTCLVKGFYPSDIAVEWESNGQPENNYKTT |
| | | | | PPVLDSDGSFFLYSKLTVDKSRWQQGNVFSCSV |
| | | | | MHEALHNHYTQKSLSLSPGK |
| 2406 | HC | H1-A118C | 80 | EVQLVESGGGLIQPGGSLRLSCAASGIDLSSST |
| | | | | MAWVRQAPGKGLEWIGIIASSGSAYYAGWAKGR |
| | | | | FTISRDNSKNTLYLQMNSLRAEDTAVYYCVRQV |
| | | | | PGYNDNRYVWGQGTLVTVSSCSTKGPSVFPLAP |
| | | | | SSKSTSGGTAALGCLVKDYFPEPVTVSWNSGAL |
| | | | | TSGVHTFPAVLQSSGLYSLSSVVTVPSSSLGTQ |
| | | | | TYICNVNHKPSNTKVDKKVEPKSCDKTHTCPPC |
| | | | | PAPELLGGPSVFLFPPKPKDTLMISRTPEVTCV |
| | | | | VVDVSHEDPEVKFNWYVDGVEVHNAKTKPREEQ |
| | | | | YNSTYRVVSVLTVLHQDWLNGKEYKCKVSNKAL |
| | | | | PAPIEKTISKAKGQPREPQVYTLPPSRDELTKN |
| | | | | QVSLTCLVKGFYPSDIAVEWESNGQPENNYKTT |
| | | | | PPVLDSDGSFFLYSKLTVDKSRWQQGNVFSCSV |
| | | | | MHEALHNHYTQKSLSLSPGK |
| 2406 | HC | H1-14AAS | 81 | EVQLVESGGGLIQPGGSLRLSCAASGIDLSSST |
| | | | | MAWVRQAPGKGLEWIGIIASSGSAYYAGWAKGR |
| | | | | FTISRDNSKNTLYLQMNSLRAEDTAVYYCVRQV |
| | | | | PGYNDNRYVWGQGTLVTVSSASTKGPSVFPLAP |
| | | | | SSKSTSGGTAALGCLVKDYFPEPVTVSWNSGAL |
| | | | | TSGVHTFPAVLQSSGLYSLSSVVTVPSSSLGTQ |
| | | | | TYICNVNHKPSNTKVDKKVEPKSCDKTHTCPPC |
| | | | | PAPEAAGGSSVFLFPPKPKDTLMISRTPEVTCV |
| | | | | VVDVSQEDPEVQFNWYVDGVEVHNAKTKPREEQ |
| | | | | YNSTYRVVSVLTVLHQDWLNGKEYKCKVSNKAL |
| | | | | PAPIEKTISKAKGQPREPQVYTLPPSRDELTKN |
| | | | | QVSLTCLVKGFYPSDIAVEWESNGQPENNYKTT |
| | | | | PPVLDSDGSFFLYSKLTVDKSRWQQGNVFSCSV |
| | | | | MHEALHNHYTQKSLSLSPGK |
| 2406 | HC | H1-14AAS- | 82 | EVQLVESGGGLIQPGGSLRLSCAASGIDLSSST |
| | | A118C | | MAWVRQAPGKGLEWIGIIASSGSAYYAGWAKGR |
| | | | | FTISRDNSKNTLYLQMNSLRAEDTAVYYCVRQV |
| | | | | PGYNDNRYVWGQGTLVTVSSCSTKGPSVFPLAP |
| | | | | SSKSTSGGTAALGCLVKDYFPEPVTVSWNSGAL |
| | | | | TSGVHTFPAVLQSSGLYSLSSVVTVPSSSLGTQ |
| | | | | TYICNVNHKPSNTKVDKKVEPKSCDKTHTCPPC |
| | | | | PAPEAAGGSSVFLFPPKPKDTLMISRTPEVTCV |
| | | | | VVDVSQEDPEVQFNWYVDGVEVHNAKTKPREEQ |
| | | | | YNSTYRVVSVLTVLHQDWLNGKEYKCKVSNKAL |
| | | | | PAPIEKTISKAKGQPREPQVYTLPPSRDELTKN |
| | | | | QVSLTCLVKGFYPSDIAVEWESNGQPENNYKIT |
| | | | | PPVLDSDGSFFLYSKLTVDKSRWQQGNVFSCSV |
| | | | | MHEALHNHYTQKSLSLSPGK |
| 2406 | HC | H1 (-K447) | 123 | EVQLVESGGGLIQPGGSLRLSCAASGIDLSSST |
| | | | | MAWVRQAPGKGLEWIGIIASSGSAYYAGWAKGR |
| | | | | FTISRDNSKNTLYLQMNSLRAEDTAVYYCVRQV |
| | | | | PGYNDNRYVWGQGTLVTVSSASTKGPSVFPLAP |
| | | | | SSKSTSGGTAALGCLVKDYFPEPVTVSWNSGAL |
| | | | | TSGVHTFPAVLQSSGLYSLSSVVTVPSSSLGTQ |
| | | | | TYICNVNHKPSNTKVDKKVEPKSCDKTHTCPPC |
| | | | | PAPELLGGPSVFLFPPKPKDTLMISRTPEVTCV |
| | | | | VVDVSHEDPEVKFNWYVDGVEVHNAKTKPREEQ |
| | | | | YNSTYRVVSVLTVLHQDWLNGKEYKCKVSNKAL |
| | | | | PAPIEKTISKAKGQPREPQVYTLPPSRDELTKN |
| | | | | QVSLTCLVKGFYPSDIAVEWESNGQPENNYKTT |
| | | | | PPVLDSDGSFFLYSKLTVDKSRWQQGNVFSCSV |
| | | | | MHEALHNHYTQKSLSLSPG |
| 2406 | HC | H1-A118C | 124 | EVQLVESGGGLIQPGGSLRLSCAASGIDLSSST |
| | | (-K447) | | MAWVRQAPGKGLEWIGIIASSGSAYYAGWAKGR |
| | | | | FTISRDNSKNTLYLQMNSLRAEDTAVYYCVRQV |
| | | | | PGYNDNRYVWGQGTLVTVSSCSTKGPSVFPLAP |
| | | | | SSKSTSGGTAALGCLVKDYFPEPVTVSWNSGAL |
| | | | | TSGVHTFPAVLQSSGLYSLSSVVTVPSSSLGTQ |
| | | | | TYICNVNHKPSNTKVDKKVEPKSCDKTHTCPPC |
| | | | | PAPELLGGPSVFLFPPKPKDTLMISRTPEVTCV |
| | | | | VVDVSHEDPEVKFNWYVDGVEVHNAKTKPREEQ |
| | | | | YNSTYRVVSVLTVLHQDWLNGKEYKCKVSNKAL |
| | | | | PAPIEKTISKAKGQPREPQVYTLPPSRDELTKN |
| | | | | QVSLTCLVKGFYPSDIAVEWESNGQPENNYKIT |
| | | | | PPVLDSDGSFFLYSKLTVDKSRWQQGNVFSCSV |
| | | | | MHEALHNHYTQKSLSLSPG |
| 2406 | HC | H1-14AAS | 125 | EVQLVESGGGLIQPGGSLRLSCAASGIDLSSST |
| | | (-K447) | | MAWVRQAPGKGLEWIGIIASSGSAYYAGWAKGR |
| | | | | FTISRDNSKNTLYLQMNSLRAEDTAVYYCVRQV |
| | | | | PGYNDNRYVWGQGTLVTVSSASTKGPSVFPLAP |
| | | | | SSKSTSGGTAALGCLVKDYFPEPVTVSWNSGAL |
| | | | | TSGVHTFPAVLQSSGLYSLSSVVTVPSSSLGTQ |
| | | | | TYICNVNHKPSNTKVDKKVEPKSCDKTHTCPPC |
| | | | | PAPEAAGGSSVFLFPPKPKDTLMISRTPEVTCV |
| | | | | VVDVSQEDPEVQFNWYVDGVEVHNAKTKPREEQ |
| | | | | YNSTYRVVSVLTVLHQDWLNGKEYKCKVSNKAL |
| | | | | PAPIEKTISKAKGQPREPQVYTLPPSRDELTKN |
| | | | | QVSLTCLVKGFYPSDIAVEWESNGQPENNYKIT |
| | | | | PPVLDSDGSFFLYSKLTVDKSRWQQGNVFSCSV |
| | | | | MHEALHNHYTQKSLSLSPG |
| 2406 | HC | H1-14AAS- | 126 | EVQLVESGGGLIQPGGSLRLSCAASGIDLSSST |
| | | A118C | | MAWVRQAPGKGLEWIGIIASSGSAYYAGWAKGR |
| | | (-K447) | | FTISRDNSKNTLYLQMNSLRAEDTAVYYCVRQV |
| | | | | PGYNDNRYVWGQGTLVTVSSCSTKGPSVFPLAP |
| | | | | SSKSTSGGTAALGCLVKDYFPEPVTVSWNSGAL |
| | | | | TSGVHTFPAVLQSSGLYSLSSVVTVPSSSLGTQ |
| | | | | TYICNVNHKPSNTKVDKKVEPKSCDKTHTCPPC |
| | | | | PAPEAAGGSSVFLFPPKPKDTLMISRTPEVTCV |
| | | | | VVDVSQEDPEVQFNWYVDGVEVHNAKTKPREEQ |
| | | | | YNSTYRVVSVLTVLHQDWLNGKEYKCKVSNKAL |
| | | | | PAPIEKTISKAKGQPREPQVYTLPPSRDELTKN |
| | | | | QVSLTCLVKGFYPSDIAVEWESNGQPENNYKTT |
| | | | | PPVLDSDGSFFLYSKLTVDKSRWQQGNVFSCSV |
| | | | | MHEALHNHYTQKSLSLSPG |
| 2406 | LC | L1 | 83 | AIVMTQSPSSLSASVGDRVTITCQASESVYNAN |
| | | | | SCSWYQQKPGKAPKLLIYDASDLASGVPSRFSG |
| | | | | SGSGTDFTLTISSLQPEDFATYYCAGYKGNGDA |
| | | | | AFGQGTKVEIKRTVAAPSVFIFPPSDEQLKSGT |
| | | | | ASVVCLLNNFYPREAKVQWKVDNALQSGNSQES |
| | | | | VTEQDSKDSTYSLSSTLTLSKADYEKHKVYACE |
| | | | | VTHQGLSSPVTKSENRGEC |
| 2406 | ILC | L1-C80 | 84 | AIVMTQSPSSLSASVGDRVTITCQASESVYNAN |
| | | | | SCSWYQQKPGKAPKLLIYDASDLASGVPSRFSG |
| | | | | SGSGTDFTLTISSLQCEDVATYYCAGYKGNGDA |
| | | | | AFGQGTKVEIKRTVAAPSVFIFPPSDEQLKSGT |
| | | | | ASVVCLLNNFYPREAKVQWKVDNALQSGNSQES |
| | | | | VTEQDSKDSTYSLSSTLTLSKADYEKHKVYACE |
| | | | | VTHQGLSSPVTKSENRGEC |

TABLE 9
Nucleic acid sequences of full-length mAB Ig chains

| mAb | IgG chain | Variant | SEQ ID NO | Nucleic acid sequence |
|---|---|---|---|---|
| 190K12 | HC | H4 | 190 | CAAGTGCAGCTTGTGCAGTCCGGAGCTGAAGTCA |
| | | | | AGAAGCCTGGTGCCAGCGTGAAAGTGTCCTGCAA |
| | | | | GGCCTCCGGATACACCTTCACCACGTACAACATG |
| | | | | CATTGGGTCAGACAGGCACCGGGACAGGGTCTGG |
| | | | | AATGGATGGGGGCCATTTTTCCCGGAAACGGCCA |
| | | | | CATCTCGTACGCCCAAAAGTTCCAGGGCCGCGTG |
| | | | | ACCATGACTGTGGACACCTCCACCTCCACCGTGT |
| | | | | ACATGGAGCTCTCAAGCCTGAGGTCCGAGGATAC |
| | | | | TGCCGTGTACTATTGTGCGCGGGGGGATTACGAC |
| | | | | GAAAGCTACTTCGACTACTGGGGCCAGGGCACTC |
| | | | | TGGTCACCGTGTCGTCAGCATCCACCAAGGGCCC |
| | | | | ATCGGTCTTCCCCCTGGCACCCTCCTCCAAGAGC |
| | | | | ACCTCTGGGGGCACAGCGGCCCTGGGCTGCCTGG |
| | | | | TCAAGGACTACTTCCCCGAACCGGTGACGGTGTC |
| | | | | GTGGAACTCAGGCGCCCTGACCAGCGGCGTGCAC |
| | | | | ACCTTCCCGGCTGTCCTACAGTCCTCAGGACTCT |
| | | | | ACTCCCTCAGCAGCGTGGTGACCGTGCCCTCCAG |
| | | | | CAGCTTGGGCACCCAGACCTACATCTGCAACGTG |
| | | | | AATCACAAGCCCAGCAACACCAAGGTGGACAAGA |
| | | | | AAGTTGAGCCCAAATCTTGTGACAAAACTCACAC |
| | | | | ATGCCCACCGTGCCCAGCACCTGAACTCCTGGGG |
| | | | | GGACCGTCAGTCTTCCTCTTCCCCCCAAAACCCA |
| | | | | AGGACACCCTCATGATCTCCCGGACCCCTGAGGT |
| | | | | CACATGCGTGGTGGTGGACGTGAGCCACGAAGAC |
| | | | | CCTGAGGTCAAGTTCAACTGGTACGTGGACGGCG |
| | | | | TGGAGGTGCATAATGCCAAGACAAAGCCGCGGGA |
| | | | | GGAGCAGTACAACAGCACGTACCGTGTGGTCAGC |
| | | | | GTCCTCACCGTCCTGCACCAGGACTGGCTGAATG |
| | | | | GCAAGGAGTACAAGTGCAAGGTCTCCAACAAAGC |
| | | | | CCTCCCAGCCCCCATCGAGAAAACCATCTCCAAA |
| | | | | GCCAAAGGGCAGCCCCGAGAACCACAGGTGTACA |
| | | | | CCCTGCCCCCATCCCGGGATGAGCTGACCAAGAA |
| | | | | CCAGGTCAGCCTGACCTGCCTGGTCAAAGGCTTC |
| | | | | TATCCCAGCGACATCGCCGTGGAGTGGGAGAGCA |
| | | | | ATGGGCAGCCGGAGAACAACTACAAGACCACGCC |
| | | | | TCCCGTGCTGGACTCCGACGGCTCCTTCTTCTTA |
| | | | | TATTCAAAGCTCACCGTGGACAAGAGCAGGTGGC |
| | | | | AGCAGGGGAACGTCTTCTCATGCTCCGTGATGCA |
| | | | | TGAGGCTCTGCACAACCACTACACGCAGAAGAGC |
| | | | | CTCTCCCTGTCTCCCGGGAAA |
| 190K12 | HC | H4-A118C | 191 | CAGGCTTATCTTCAGCAGTCTGGGGCTGCGCTGG |
| | | | | TGAGGCCTGGGGCCTCAGTGAAGATGTCCTGCAA |
| | | | | GTCTTCTGGCTACACATTTACCACTTACAATATA |
| | | | | CACTGGGTAAAGCAGACACCTAGACAGGGCCTGG |
| | | | | AATGGATTGGAGCTATTTTTCCAGGAAATGGTCA |
| | | | | TATTTCCTACAATCAGAAGTTCAAGGGCAAGGCC |
| | | | | ACACTGACTGTAGACAAATCCTCCAACACAGCCT |
| | | | | ACATGCAGCTCAGCAGCCTGACATCTGAAGACTC |
| | | | | TGCGGTCTATTTCTGTGCAAGAGGGGATTACGAC |
| | | | | GAGTCTTACTTTGACTACTGGGGCCAAGGCACCA |
| | | | | CTCTCACAGTCTCCTCATGCTCCACCAAGGGCCC |
| | | | | ATCGGTCTTCCCCCTGGCACCCTCCTCCAAGAGC |
| | | | | ACCTCTGGGGGCACAGCGGCCCTGGGCTGCCTGG |
| | | | | TCAAGGACTACTTCCCCGAACCGGTGACGGTGTC |
| | | | | GTGGAACTCAGGCGCCCTGACCAGCGGCGTGCAC |
| | | | | ACCTTCCCGGCTGTCCTACAGTCCTCAGGACTCT |
| | | | | ACTCCCTCAGCAGCGTGGTGACCGTGCCCTCCAG |
| | | | | CAGCTTGGGCACCCAGACCTACATCTGCAACGTG |
| | | | | AATCACAAGCCCAGCAACACCAAGGTGGACAAGA |
| | | | | AAGTTGAGCCCAAATCTTGTGACAAAACTCACAC |
| | | | | ATGCCCACCGTGCCCAGCACCTGAACTCCTGGGG |
| | | | | GGACCGTCAGTCTTCCTCTTCCCCCCAAAACCCA |
| | | | | AGGACACCCTCATGATCTCCCGGACCCCTGAGGT |
| | | | | CACATGCGTGGTGGTGGACGTGAGCCACGAAGAC |
| | | | | CCTGAGGTCAAGTTCAACTGGTACGTGGACGGCG |
| | | | | TGGAGGTGCATAATGCCAAGACAAAGCCGCGGGA |
| | | | | GGAGCAGTACAACAGCACGTACCGTGTGGTCAGC |
| | | | | GTCCTCACCGTCCTGCACCAGGACTGGCTGAATG |
| | | | | GCAAGGAGTACAAGTGCAAGGTCTCCAACAAAGC |
| | | | | CCTCCCAGCCCCCATCGAGAAAACCATCTCCAAA |
| | | | | GCCAAAGGGCAGCCCCGAGAACCACAGGTGTACA |
| | | | | CCCTGCCCCCATCCCGGGATGAGCTGACCAAGAA |
| | | | | CCAGGTCAGCCTGACCTGCCTGGTCAAAGGCTTC |
| | | | | TATCCCAGCGACATCGCCGTGGAGTGGGAGAGCA |
| | | | | ATGGGCAGCCGGAGAACAACTACAAGACCACGCC |
| | | | | TCCCGTGCTGGACTCCGACGGCTCCTTCTTCTTA |
| | | | | TATTCAAAGCTCACCGTGGACAAGAGCAGGTGGC |
| | | | | AGCAGGGGAACGTCTTCTCATGCTCCGTGATGCA |
| | | | | TGAGGCTCTGCACAACCACTACACGCAGAAGAGC |
| | | | | CTCTCCCTGTCTCCCGGGAAA |
| 190K12 | HC | H4-14AAS | 192 | CAAGTGCAGCTTGTGCAGTCCGGAGCTGAAGTCA |
| | | | | AGAAGCCTGGTGCCAGCGTGAAAGTGTCCTGCAA |
| | | | | GGCCTCCGGATACACCTTCACCACGTACAACATG |
| | | | | CATTGGGTCAGACAGGCACCGGGACAGGGTCTGG |
| | | | | AATGGATGGGGGCCATTTTTCCCGGAAACGGCCA |
| | | | | CATCTCGTACGCCCAAAAGTTCCAGGGCCGCGTG |
| | | | | ACCATGACTGTGGACACCTCCACCTCCACCGTGT |
| | | | | ACATGGAGCTCTCAAGCCTGAGGTCCGAGGATAC |
| | | | | TGCCGTGTACTATTGTGCGCGGGGGGATTACGAC |
| | | | | GAAAGCTACTTCGACTACTGGGGCCAGGGCACTC |
| | | | | TGGTCACCGTGTCGTCAGCATCCACCAAGGGCCC |
| | | | | ATCGGTCTTCCCCCTGGCACCCTCCTCCAAGAGC |
| | | | | ACCTCTGGGGGCACAGCGGCCCTGGGCTGCCTGG |
| | | | | TCAAGGACTACTTCCCCGAACCGGTGACGGTGTC |
| | | | | GTGGAACTCAGGCGCCCTGACCAGCGGCGTGCAC |
| | | | | ACCTTCCCGGCTGTCCTACAGTCCTCAGGACTCT |
| | | | | ACTCCCTCAGCAGCGTGGTGACCGTGCCCTCCAG |
| | | | | CAGCTTGGGCACCCAGACCTACATCTGCAACGTG |
| | | | | AATCACAAGCCCAGCAACACCAAGGTGGACAAGA |
| | | | | AAGTTGAGCCCAAATCTTGTGACAAAACTCACAC |
| | | | | ATGCCCACCGTGCCCAGCACCTGAGGCCGCCGGG |
| | | | | GGATCCTCAGTCTTCCTGTTCCCCCCAAAACCCA |
| | | | | AGGACACTCTCATGATCTCCCGGACCCCTGAGGT |
| | | | | CACGTGCGTGGTGGTGGACGTGAGCCAGGAAGAC |
| | | | | CCCGAGGTCCAGTTCAACTGGTACGTGGATGGCG |
| | | | | TGGAGGTGCATAATGCCAAGACAAAGCCGCGGGA |
| | | | | GGAGCAGTACAACAGCACGTACCGTGTGGTCAGC |
| | | | | GTCCTCACCGTCCTGCACCAGGACTGGCTGAACG |
| | | | | GCAAGGAGTACAAGTGCAAGGTCTCCAACAAAGC |
| | | | | CCTCCCGGCCCCCATCGAGAAAACCATCTCCAAA |
| | | | | GCCAAAGGGCAGCCCCGAGAACCACAGGTGTACA |
| | | | | CCCTGCCCCCATCCCGGGATGAGCTGACCAAGAA |
| | | | | CCAGGTCAGCCTGACCTGCCTGGTCAAAGGCTTC |
| | | | | TATCCCAGCGACATCGCCGTGGAGTGGGAGAGCA |
| | | | | ATGGGCAGCCGGAGAACAACTACAAGACCACGCC |
| | | | | TCCCGTGCTGGACTCCGACGGCTCCTTCTTCTTA |
| | | | | TATTCAAAGCTCACCGTGGACAAGAGCAGGTGGC |
| | | | | AGCAGGGGAACGTCTTCTCATGCTCCGTGATGCA |
| | | | | TGAGGCTCTGCACAACCACTACACGCAGAAGAGC |
| | | | | CTCTCCCTGTCTCCCGGGAAA |
| 190K12 | HC | H4-14AAS- | 193 | CAAGTGCAGCTTGTGCAGTCCGGAGCTGAAGTCA |
| | | A118C | | AGAAGCCTGGTGCCAGCGTGAAAGTGTCCTGCAA |
| | | | | GGCCTCCGGATACACCTTCACCACGTACAACATG |
| | | | | CATTGGGTCAGACAGGCACCGGGACAGGGTCTGG |
| | | | | AATGGATGGGGGCCATTTTTCCCGGAAACGGCCA |
| | | | | CATCTCGTACGCCCAAAAGTTCCAGGGCCGCGTG |
| | | | | ACCATGACTGTGGACACCTCCACCTCCACCGTGT |
| | | | | ACATGGAGCTCTCAAGCCTGAGGTCCGAGGATAC |
| | | | | TGCCGTGTACTATTGTGCGCGGGGGGATTACGAC |
| | | | | GAAAGCTACTTCGACTACTGGGGCCAGGGCACTC |
| | | | | TGGTCACCGTGTCGTCATGCTCCACCAAGGGCCC |
| | | | | ATCGGTCTTCCCCCTGGCACCCTCCTCCAAGAGC |
| | | | | ACCTCTGGGGGCACAGCGGCCCTGGGCTGCCTGG |
| | | | | TCAAGGACTACTTCCCCGAACCGGTGACGGTGTC |
| | | | | GTGGAACTCAGGCGCCCTGACCAGCGGCGTGCAC |
| | | | | ACCTTCCCGGCTGTCCTACAGTCCTCAGGACTCT |
| | | | | ACTCCCTCAGCAGCGTGGTGACCGTGCCCTCCAG |
| | | | | CAGCTTGGGCACCCAGACCTACATCTGCAACGTG |
| | | | | AATCACAAGCCCAGCAACACCAAGGTGGACAAGA |
| | | | | AAGTTGAGCCCAAATCTTGTGACAAAACTCACAC |
| | | | | ATGCCCACCGTGCCCAGCACCTGAGGCCGCCGGG |
| | | | | GGATCCTCAGTCTTCCTGTTCCCCCCAAAACCCA |
| | | | | AGGACACTCTCATGATCTCCCGGACCCCTGAGGT |
| | | | | CACGTGCGTGGTGGTGGACGTGAGCCAGGAAGAC |
| | | | | CCCGAGGTCCAGTTCAACTGGTACGTGGATGGCG |
| | | | | TGGAGGTGCATAATGCCAAGACAAAGCCGCGGGA |
| | | | | GGAGCAGTACAACAGCACGTACCGTGTGGTCAGC |
| | | | | GTCCTCACCGTCCTGCACCAGGACTGGCTGAACG |
| | | | | GCAAGGAGTACAAGTGCAAGGTCTCCAACAAAGC |
| | | | | CCTCCCGGCCCCCATCGAGAAAACCATCTCCAAA |
| | | | | GCCAAAGGGCAGCCCCGAGAACCACAGGTGTACA |
| | | | | CCCTGCCCCCATCCCGGGATGAGCTGACCAAGAA |
| | | | | CCAGGTCAGCCTGACCTGCCTGGTCAAAGGCTTC |
| | | | | TATCCCAGCGACATCGCCGTGGAGTGGGAGAGCA |
| | | | | ATGGGCAGCCGGAGAACAACTACAAGACCACGCC |
| | | | | TCCCGTGCTGGACTCCGACGGCTCCTTCTTCTTA |
| | | | | TATTCAAAGCTCACCGTGGACAAGAGCAGGTGGC |
| | | | | AGCAGGGGAACGTCTTCTCATGCTCCGTGATGCA |
| | | | | TGAGGCTCTGCACAACCACTACACGCAGAAGAGC |
| | | | | CTCTCCCTGTCTCCCGGGAAA |
| 190K12 | LC | L1 | 194 | GACATTCAGATGACCCAATCCCCCTCATCACTGT |
| | | | | CGGCTTCCGTGGGCGACCGCGTGACCATCACCTG |
| | | | | TAAAGCCTCCGAGGGGATCTACAACAGACTCGCC |
| | | | | TGGTATCAGCAGAAGCCAGGAAAGGCCCCTAAGC |
| | | | | TCCTTCTGTACGGAGTGATCTCCCTGGAAACTGG |
| | | | | AGTGCCGAGCCGGTTTAGCGGCAGCGGTTCCGGA |
| | | | | ACTGACTACACGTTGACCATCTCCTCGCTGCAAC |
| | | | | CCGAAGATTTCGCGACCTACTACTGCCAGCAGTA |
| | | | | CTGGTCGATTCCGTGGACTTTCGGTGGCGGGACC |
| | | | | AAGGTCGAGATCAAAAGAACAGTGGCCGCTCCGA |
| | | | | GCGTGTTCATCTTTCCACCAAGCGACGAGCAGCT |
| | | | | GAAAAGCGGCACAGCCTCTGTCGTGTGCCTGCTG |
| | | | | AACAACTTCTACCCCAGAGAAGCCAAGGTGCAGT |
| | | | | GGAAGGTGGACAATGCCCTGCAGAGCGGCAATAG |
| | | | | CCAAGAGAGCGTGACCGAGCAGGACAGCAAGGAT |
| | | | | AGCACCTACAGCCTGTCCAGCACACTGACCCTGA |
| | | | | GCAAGGCCGACTACGAGAAGCACAAAGTGTACGC |
| | | | | CTGCGAAGTGACACACCAGGGCCTGTCTAGCCCT |
| | | | | GTGACCAAGAGCTTCAACCGGGGCGAGTGT |
| 190K12 | LC | L1-C80 | 195 | GATATTCAGATGACACAGAGCCCTAGCAGCCTGA |
| | | | | GCGCCTCTGTGGGCGATAGAGTGACAATCACATG |
| | | | | CAAGGCCAGCGAGGGCATCTACAACAGACTGGCC |
| | | | | TGGTATCAGCAGAAGCCCGGAAAGGCTCCTAAGC |
| | | | | TGCTGCTGTACGGCGTGATCTCTCTGGAAACAGG |
| | | | | CGTGCCAAGCAGATTTTCTGGCAGCGGCTCTGGC |
| | | | | ACCGACTACACCCTGACAATTTCTAGCCTGCAGT |
| | | | | GCGAGGACGTGGCCACCTACTACTGCCAGCAGTA |
| | | | | CTGGTCTATCCCCTGGACCTTTGGCGGAGGCACC |
| | | | | AAGGTGGAAATCAAGAGAACAGTGGCCGCTCCGA |
| | | | | GCGTGTTCATCTTTCCACCAAGCGACGAGCAGCT |
| | | | | GAAAAGCGGCACAGCCTCTGTCGTGTGCCTGCTG |
| | | | | AACAACTTCTACCCCAGAGAAGCCAAGGTGCAGT |
| | | | | GGAAGGTGGACAATGCCCTGCAGAGCGGCAATAG |
| | | | | CCAAGAGAGCGTGACCGAGCAGGACAGCAAGGAT |
| | | | | AGCACCTACAGCCTGTCCAGCACACTGACCCTGA |
| | | | | GCAAGGCCGACTACGAGAAGCACAAAGTGTACGC |
| | | | | CTGCGAAGTGACACACCAGGGCCTGTCTAGCCCT |
| | | | | GTGACCAAGAGCTTCAACCGGGGCGAGTGT |
| 366F5 | HC | H2 | 196 | CAAGTGCAGCTGGTGCAGTCTGGTGCCGAGGTCA |
| | | | | AGAAGCCAGGCTCCAGCGTGAAAGTGTCCTGCAA |
| | | | | GGCCTCGGGATACGCATTCCGGTCGTACTGGATC |
| | | | | TCCTGGGTCCGCCAGGCCCCCGGACAAGGCCTTG |
| | | | | AATGGATGGGGCAGATCTTTCCTGGGGACGGCGA |
| | | | | CATTAACTACGCGCAGAAGTTCCAGGGCAGAGTG |
| | | | | ACCATCACTGCCGATAAGAGCACCTCCACTGCGT |
| | | | | ACATGGAACTGAGCTCCCTGAGGTCCGAGGATAC |
| | | | | CGCCGTGTACTATTGCGCTCGGATGGACTTCTAC |
| | | | | TCCTGGTTCCCGTACTGGGGACAGGGTACCCTCG |
| | | | | TCACCGTGTCATCAGCATCCACCAAGGGCCCATC |
| | | | | GGTCTTCCCCCTGGCACCCTCCTCCAAGAGCACC |
| | | | | TCTGGGGGCACAGCGGCCCTGGGCTGCCTGGTCA |
| | | | | AGGACTACTTCCCCGAACCGGTGACGGTGTCGTG |
| | | | | GAACTCAGGCGCCCTGACCAGCGGCGTGCACACC |
| | | | | TTCCCGGCTGTCCTACAGTCCTCAGGACTCTACT |
| | | | | CCCTCAGCAGCGTGGTGACCGTGCCCTCCAGCAG |
| | | | | CTTGGGCACCCAGACCTACATCTGCAACGTGAAT |
| | | | | CACAAGCCCAGCAACACCAAGGTGGACAAGAAAG |
| | | | | TTGAGCCCAAATCTTGTGACAAAACTCACACATG |
| | | | | CCCACCGTGCCCAGCACCTGAACTCCTGGGGGGA |
| | | | | CCGTCAGTCTTCCTCTTCCCCCCAAAACCCAAGG |
| | | | | ACACCCTCATGATCTCCCGGACCCCTGAGGTCAC |
| | | | | ATGCGTGGTGGTGGACGTGAGCCACGAAGACCCT |
| | | | | GAGGTCAAGTTCAACTGGTACGTGGACGGCGTGG |
| | | | | AGGTGCATAATGCCAAGACAAAGCCGCGGGAGGA |
| | | | | GCAGTACAACAGCACGTACCGTGTGGTCAGCGTC |
| | | | | CTCACCGTCCTGCACCAGGACTGGCTGAATGGCA |
| | | | | AGGAGTACAAGTGCAAGGTCTCCAACAAAGCCCT |
| | | | | CCCAGCCCCCATCGAGAAAACCATCTCCAAAGCC |
| | | | | AAAGGGCAGCCCCGAGAACCACAGGTGTACACCC |
| | | | | TGCCCCCATCCCGGGATGAGCTGACCAAGAACCA |
| | | | | GGTCAGCCTGACCTGCCTGGTCAAAGGCTTCTAT |
| | | | | CCCAGCGACATCGCCGTGGAGTGGGAGAGCAATG |
| | | | | GGCAGCCGGAGAACAACTACAAGACCACGCCTCC |
| | | | | CGTGCTGGACTCCGACGGCTCCTTCTTCTTATAT |
| | | | | TCAAAGCTCACCGTGGACAAGAGCAGGTGGCAGC |
| | | | | AGGGGAACGTCTTCTCATGCTCCGTGATGCATGA |
| | | | | GGCTCTGCACAACCACTACACGCAGAAGAGCCTC |
| | | | | TCCCTGTCTCCCGGGAAA |
| 366F5 | HC | H2-A118C | 197 | CAAGTGCAGCTGGTGCAGTCTGGTGCCGAGGTCA |
| | | | | AGAAGCCAGGCTCCAGCGTGAAAGTGTCCTGCAA |
| | | | | GGCCTCGGGATACGCATTCCGGTCGTACTGGATC |
| | | | | TCCTGGGTCCGCCAGGCCCCCGGACAAGGCCTTG |
| | | | | AATGGATGGGGCAGATCTTTCCTGGGGACGGCGA |
| | | | | CATTAACTACGCGCAGAAGTTCCAGGGCAGAGTG |
| | | | | ACCATCACTGCCGATAAGAGCACCTCCACTGCGT |
| | | | | ACATGGAACTGAGCTCCCTGAGGTCCGAGGATAC |
| | | | | CGCCGTGTACTATTGCGCTCGGATGGACTTCTAC |
| | | | | TCCTGGTTCCCGTACTGGGGACAGGGTACCCTCG |
| | | | | TCACCGTGTCATCATGCTCCACCAAGGGCCCATC |
| | | | | GGTCTTCCCCCTGGCACCCTCCTCCAAGAGCACC |
| | | | | TCTGGGGGCACAGCGGCCCTGGGCTGCCTGGTCA |
| | | | | AGGACTACTTCCCCGAACCGGTGACGGTGTCGTG |
| | | | | GAACTCAGGCGCCCTGACCAGCGGCGTGCACACC |
| | | | | TTCCCGGCTGTCCTACAGTCCTCAGGACTCTACT |
| | | | | CCCTCAGCAGCGTGGTGACCGTGCCCTCCAGCAG |
| | | | | CTTGGGCACCCAGACCTACATCTGCAACGTGAAT |
| | | | | CACAAGCCCAGCAACACCAAGGTGGACAAGAAAG |
| | | | | TTGAGCCCAAATCTTGTGACAAAACTCACACATG |
| | | | | CCCACCGTGCCCAGCACCTGAACTCCTGGGGGGA |
| | | | | CCGTCAGTCTTCCTCTTCCCCCCAAAACCCAAGG |
| | | | | ACACCCTCATGATCTCCCGGACCCCTGAGGTCAC |
| | | | | ATGCGTGGTGGTGGACGTGAGCCACGAAGACCCT |
| | | | | GAGGTCAAGTTCAACTGGTACGTGGACGGCGTGG |
| | | | | AGGTGCATAATGCCAAGACAAAGCCGCGGGAGGA |
| | | | | GCAGTACAACAGCACGTACCGTGTGGTCAGCGTC |
| | | | | CTCACCGTCCTGCACCAGGACTGGCTGAATGGCA |
| | | | | AGGAGTACAAGTGCAAGGTCTCCAACAAAGCCCT |
| | | | | CCCAGCCCCCATCGAGAAAACCATCTCCAAAGCC |
| | | | | AAAGGGCAGCCCCGAGAACCACAGGTGTACACCC |
| | | | | TGCCCCCATCCCGGGATGAGCTGACCAAGAACCA |
| | | | | GGTCAGCCTGACCTGCCTGGTCAAAGGCTTCTAT |
| | | | | CCCAGCGACATCGCCGTGGAGTGGGAGAGCAATG |
| | | | | GGCAGCCGGAGAACAACTACAAGACCACGCCTCC |
| | | | | CGTGCTGGACTCCGACGGCTCCTTCTTCTTATAT |
| | | | | TCAAAGCTCACCGTGGACAAGAGCAGGTGGCAGC |
| | | | | AGGGGAACGTCTTCTCATGCTCCGTGATGCATGA |
| | | | | GGCTCTGCACAACCACTACACGCAGAAGAGCCTC |
| | | | | TCCCTGTCTCCCGGGAAA |
| 366F5 | HC | H2-14AAS | 198 | CAAGTGCAGCTGGTGCAGTCTGGTGCCGAGGTCA |
| | | | | AGAAGCCAGGCTCCAGCGTGAAAGTGTCCTGCAA |
| | | | | GGCCTCGGGATACGCATTCCGGTCGTACTGGATC |
| | | | | TCCTGGGTCCGCCAGGCCCCCGGACAAGGCCTTG |
| | | | | AATGGATGGGGCAGATCTTTCCTGGGGACGGCGA |
| | | | | CATTAACTACGCGCAGAAGTTCCAGGGCAGAGTG |
| | | | | ACCATCACTGCCGATAAGAGCACCTCCACTGCGT |
| | | | | ACATGGAACTGAGCTCCCTGAGGTCCGAGGATAC |
| | | | | CGCCGTGTACTATTGCGCTCGGATGGACTTCTAC |
| | | | | TCCTGGTTCCCGTACTGGGGACAGGGTACCCTCG |
| | | | | TCACCGTGTCATCAGCATCCACCAAGGGCCCATC |
| | | | | GGTCTTCCCCCTGGCACCCTCCTCCAAGAGCACC |
| | | | | TCTGGGGGCACAGCGGCCCTGGGCTGCCTGGTCA |
| | | | | AGGACTACTTCCCCGAACCGGTGACGGTGTCGTG |
| | | | | GAACTCAGGCGCCCTGACCAGCGGCGTGCACACC |
| | | | | TTCCCGGCTGTCCTACAGTCCTCAGGACTCTACT |
| | | | | CCCTCAGCAGCGTGGTGACCGTGCCCTCCAGCAG |
| | | | | CTTGGGCACCCAGACCTACATCTGCAACGTGAAT |
| | | | | CACAAGCCCAGCAACACCAAGGIGGACAAGAAAG |
| | | | | TTGAGCCCAAATCTTGTGACAAAACTCACACATG |
| | | | | CCCACCGTGCCCAGCACCTGAGGCCGCCGGGGGA |
| | | | | TCCTCAGTCTTCCTGTTCCCCCCAAAACCCAAGG |
| | | | | ACACTCTCATGATCTCCCGGACCCCTGAGGTCAC |
| | | | | GTGCGTGGTGGTGGACGTGAGCCAGGAAGACCCC |
| | | | | GAGGTCCAGTTCAACTGGTACGTGGATGGCGTGG |
| | | | | AGGTGCATAATGCCAAGACAAAGCCGCGGGAGGA |
| | | | | GCAGTACAACAGCACGTACCGTGTGGTCAGCGTC |
| | | | | CTCACCGTCCTGCACCAGGACTGGCTGAACGGCA |
| | | | | AGGAGTACAAGTGCAAGGTCTCCAACAAAGCCCT |
| | | | | CCCGGCCCCCATCGAGAAAACCATCTCCAAAGCC |
| | | | | AAAGGGCAGCCCCGAGAACCACAGGTGTACACCC |
| | | | | TGCCCCCATCCCGGGATGAGCTGACCAAGAACCA |
| | | | | GGTCAGCCTGACCTGCCTGGTCAAAGGCTTCTAT |
| | | | | CCCAGCGACATCGCCGTGGAGTGGGAGAGCAATG |
| | | | | GGCAGCCGGAGAACAACTACAAGACCACGCCTCC |
| | | | | CGTGCTGGACTCCGACGGCTCCTTCTTCTTATAT |
| | | | | TCAAAGCTCACCGTGGACAAGAGCAGGTGGCAGC |
| | | | | AGGGGAACGTCTTCTCATGCTCCGTGATGCATGA |
| | | | | GGCTCTGCACAACCACTACACGCAGAAGAGCCTC |
| | | | | TCCCTGTCTCCCGGGAAA |
| 366F5 | HC | H2-14AAS- | 199 | CAAGTGCAGCTGGTGCAGTCTGGTGCCGAGGTCA |
| | | A118C | | AGAAGCCAGGCTCCAGCGTGAAAGTGTCCTGCAA |
| | | | | GGCCTCGGGATACGCATTCCGGTCGTACTGGATC |
| | | | | TCCTGGGTCCGCCAGGCCCCCGGACAAGGCCTTG |
| | | | | AATGGATGGGGCAGATCTTTCCTGGGGACGGCGA |
| | | | | CATTAACTACGCGCAGAAGTTCCAGGGCAGAGTG |
| | | | | ACCATCACTGCCGATAAGAGCACCTCCACTGCGT |
| | | | | ACATGGAACTGAGCTCCCTGAGGTCCGAGGATAC |
| | | | | CGCCGTGTACTATTGCGCTCGGATGGACTTCTAC |
| | | | | TCCTGGTTCCCGTACTGGGGACAGGGTACCCTCG |
| | | | | TCACCGTGTCATCATGCTCCACCAAGGGCCCATC |
| | | | | GGTCTTCCCCCTGGCACCCTCCTCCAAGAGCACC |
| | | | | TCTGGGGGCACAGCGGCCCTGGGCTGCCTGGTCA |
| | | | | AGGACTACTTCCCCGAACCGGTGACGGTGTCGTG |
| | | | | GAACTCAGGCGCCCTGACCAGCGGCGTGCACACC |
| | | | | TTCCCGGCTGTCCTACAGTCCTCAGGACTCTACT |
| | | | | CCCTCAGCAGCGTGGTGACCGTGCCCTCCAGCAG |
| | | | | CTTGGGCACCCAGACCTACATCTGCAACGTGAAT |
| | | | | CACAAGCCCAGCAACACCAAGGTGGACAAGAAAG |
| | | | | TTGAGCCCAAATCTTGTGACAAAACTCACACATG |
| | | | | CCCACCGTGCCCAGCACCTGAGGCCGCCGGGGGA |
| | | | | TCCTCAGTCTTCCTGTTCCCCCCAAAACCCAAGG |
| | | | | ACACTCTCATGATCTCCCGGACCCCTGAGGTCAC |
| | | | | GTGCGTGGTGGTGGACGTGAGCCAGGAAGACCCC |
| | | | | GAGGTCCAGTTCAACTGGTACGTGGATGGCGTGG |
| | | | | AGGTGCATAATGCCAAGACAAAGCCGCGGGAGGA |
| | | | | GCAGTACAACAGCACGTACCGTGTGGTCAGCGTC |
| | | | | CTCACCGTCCTGCACCAGGACTGGCTGAACGGCA |
| | | | | AGGAGTACAAGTGCAAGGTCTCCAACAAAGCCCT |
| | | | | CCCGGCCCCCATCGAGAAAACCATCTCCAAAGCC |
| | | | | AAAGGGCAGCCCCGAGAACCACAGGTGTACACCC |
| | | | | TGCCCCCATCCCGGGATGAGCTGACCAAGAACCA |
| | | | | GGTCAGCCTGACCTGCCTGGTCAAAGGCTTCTAT |
| | | | | CCCAGCGACATCGCCGTGGAGTGGGAGAGCAATG |
| | | | | GGCAGCCGGAGAACAACTACAAGACCACGCCTCC |
| | | | | CGTGCTGGACTCCGACGGCTCCTTCTTCTTATAT |
| | | | | TCAAAGCTCACCGTGGACAAGAGCAGGTGGCAGC |
| | | | | AGGGGAACGTCTTCTCATGCTCCGTGATGCATGA |
| | | | | GGCTCTGCACAACCACTACACGCAGAAGAGCCTC |
| | | | | TCCCTGTCTCCCGGGAAA |
| 366F5 | LC | L1 | 200 | GACATTGTGATGACCCAGTCCCCCGATTCCCTTG |
| | | | | CCGTGTCCCTCGGTGAACGGGCCACTATCAACTG |
| | | | | CAAATCCAGCCAGTCGCTGCTGAACTCGGGGAAC |
| | | | | CAGAAGAATGATCTGGCCTGGTACCAGCAGAAGC |
| | | | | CTGGCCAGCCCCCAAAGCTGCTCATCTACGGAGC |
| | | | | ATCAACTCGCGAATCCGGAGTGCCGGACAGATTT |
| | | | | TCCGGGAGCGGATCAGGCACCGACTTCACCTTGA |
| | | | | CCATTTCGAGCCTGCAAGCGGAGGACGTCGCTGT |
| | | | | GTACTACTGTCAGAACGACCACTCCTATCCGCTG |
| | | | | ACTTTCGGACAAGGCACCAAGCTGGAGATCAAAA |
| | | | | GAACAGTGGCCGCTCCGAGCGTGTTCATCTTTCC |
| | | | | ACCAAGCGACGAGCAGCTGAAAAGCGGCACAGCC |
| | | | | TCTGTCGTGTGCCTGCTGAACAACTTCTACCCCA |
| | | | | GAGAAGCCAAGGTGCAGTGGAAGGTGGACAATGC |
| | | | | CCTGCAGAGCGGCAATAGCCAAGAGAGCGTGACC |
| | | | | GAGCAGGACAGCAAGGATAGCACCTACAGCCTGT |
| | | | | CCAGCACACTGACCCTGAGCAAGGCCGACTACGA |
| | | | | GAAGCACAAAGTGTACGCCTGCGAAGTGACACAC |
| | | | | CAGGGCCTGTCTAGCCCTGTGACCAAGAGCTTCA |
| | | | | ACCGGGGCGAGTGT |
| 366F5 | LC | L1-C80 | 201 | GACATTGTGATGACCCAGTCCCCCGATTCCCTTG |
| | | | | CCGTGTCCCTCGGTGAACGGGCCACTATCAACTG |
| | | | | CAAATCCAGCCAGTCGCTGCTGAACTCGGGGAAC |
| | | | | CAGAAGAATGATCTGGCCTGGTACCAGCAGAAGC |
| | | | | CTGGCCAGCCCCCAAAGCTGCTCATCTACGGAGC |
| | | | | ATCAACTCGCGAATCCGGAGTGCCGGACAGATTT |
| | | | | TCCGGGAGCGGATCAGGCACCGACTTCACCTTGA |
| | | | | CCATTTCGAGCCTGCAATGCGAGGACGTCGCTGT |
| | | | | GTACTACTGTCAGAACGACCACTCCTATCCGCTG |
| | | | | ACTTTCGGACAAGGCACCAAGCTGGAGATCAAAA |
| | | | | GAACAGTGGCCGCTCCGAGCGTGTTCATCTTTCC |
| | | | | ACCAAGCGACGAGCAGCTGAAAAGCGGCACAGCC |
| | | | | TCTGTCGTGTGCCTGCTGAACAACTTCTACCCCA |
| | | | | GAGAAGCCAAGGTGCAGTGGAAGGTGGACAATGC |
| | | | | CCTGCAGAGCGGCAATAGCCAAGAGAGCGTGACC |
| | | | | GAGCAGGACAGCAAGGATAGCACCTACAGCCTGT |
| | | | | CCAGCACACTGACCCTGAGCAAGGCCGACTACGA |
| | | | | GAAGCACAAAGTGTACGCCTGCGAAGTGACACAC |
| | | | | CAGGGCCTGTCTAGCCCTGTGACCAAGAGCTTCA |
| | | | | ACCGGGGCGAGTGT |
| 23P11 | HC | H1 | 202 | GAAGTGCAGCTGGTTGAATCTGGCGGAGGACTGA |
| | | | | TTCAGCCTGGCGGCTCTCTGAGACTGTCTTGTGC |
| | | | | CGCCTCTGGCTTCAGCCTGAGCAACTACTACATC |
| | | | | AACTGGGTCCGACAGGCCCCTGGCAAAGGACTGG |
| | | | | AATGGATCGGCTTCATCCCTATGTACGGCACCAC |
| | | | | ACACTACGCCAACTGGGCCAAGGGCAGATTCACC |
| | | | | ATCAGCCGGGACAACAGCAAGAACACCCTGTACC |
| | | | | TGCAGATGAACAGCCTGAGAGCCGAGGACACCGC |
| | | | | CGTGTACTATTGTGCCAGAGGAATCGCCAGCATG |
| | | | | TTCTACCCTTCTATCTGGGGCCAGGGCACCCTGG |
| | | | | TCACAGTTAGTTCTGCATCCACCAAGGGCCCATC |
| | | | | GGTCTTCCCCCTGGCACCCTCCTCCAAGAGCACC |
| | | | | TCTGGGGGCACAGCGGCCCTGGGCTGCCTGGTCA |
| | | | | AGGACTACTTCCCCGAACCGGTGACGGTGTCGTG |
| | | | | GAACTCAGGCGCCCTGACCAGCGGCGTGCACACC |
| | | | | TTCCCGGCTGTCCTACAGTCCTCAGGACTCTACT |
| | | | | CCCTCAGCAGCGTGGTGACCGTGCCCTCCAGCAG |
| | | | | CTTGGGCACCCAGACCTACATCTGCAACGTGAAT |
| | | | | CACAAGCCCAGCAACACCAAGGTGGACAAGAAAG |
| | | | | TTGAGCCCAAATCTTGTGACAAAACTCACACATG |
| | | | | CCCACCGTGCCCAGCACCTGAACTCCTGGGGGGA |
| | | | | CCGTCAGTCTTCCTCTTCCCCCCAAAACCCAAGG |
| | | | | ACACCCTCATGATCTCCCGGACCCCTGAGGTCAC |
| | | | | ATGCGTGGTGGTGGACGTGAGCCACGAAGACCCT |
| | | | | GAGGTCAAGTTCAACTGGTACGTGGACGGCGTGG |
| | | | | AGGTGCATAATGCCAAGACAAAGCCGCGGGAGGA |
| | | | | GCAGTACAACAGCACGTACCGTGTGGTCAGCGTC |
| | | | | CTCACCGTCCTGCACCAGGACTGGCTGAATGGCA |
| | | | | AGGAGTACAAGTGCAAGGTCTCCAACAAAGCCCT |
| | | | | CCCAGCCCCCATCGAGAAAACCATCTCCAAAGCC |
| | | | | AAAGGGCAGCCCCGAGAACCACAGGTGTACACCC |
| | | | | TGCCCCCATCCCGGGATGAGCTGACCAAGAACCA |
| | | | | GGTCAGCCTGACCTGCCTGGTCAAAGGCTTCTAT |
| | | | | CCCAGCGACATCGCCGTGGAGTGGGAGAGCAATG |
| | | | | GGCAGCCGGAGAACAACTACAAGACCACGCCTCC |
| | | | | CGTGCTGGACTCCGACGGCTCCTTCTTCTTATAT |
| | | | | TCAAAGCTCACCGTGGACAAGAGCAGGTGGCAGC |
| | | | | AGGGGAACGTCTTCTCATGCTCCGTGATGCATGA |
| | | | | GGCTCTGCACAACCACTACACGCAGAAGAGCCTC |
| | | | | TCCCTGTCTCCCGGGAAA |
| 23P11 | HC | H1-A118C | 203 | GAAGTGCAGCTGGTTGAATCTGGCGGAGGACTGA |
| | | | | TTCAGCCTGGCGGCTCTCTGAGACTGTCTTGTGC |
| | | | | CGCCTCTGGCTTCAGCCTGAGCAACTACTACATC |
| | | | | AACTGGGTCCGACAGGCCCCTGGCAAAGGACTGG |
| | | | | AATGGATCGGCTTCATCCCTATGTACGGCACCAC |
| | | | | ACACTACGCCAACTGGGCCAAGGGCAGATTCACC |
| | | | | ATCAGCCGGGACAACAGCAAGAACACCCTGTACC |
| | | | | TGCAGATGAACAGCCTGAGAGCCGAGGACACCGC |
| | | | | CGTGTACTATTGTGCCAGAGGAATCGCCAGCATG |
| | | | | TTCTACCCTTCTATCTGGGGCCAGGGCACCCTGG |
| | | | | TCACAGTTAGTTCTTGCTCCACCAAGGGCCCATC |
| | | | | GGTCTTCCCCCTGGCACCCTCCTCCAAGAGCACC |
| | | | | TCTGGGGGCACAGCGGCCCTGGGCTGCCTGGTCA |
| | | | | AGGACTACTTCCCCGAACCGGTGACGGTGTCGTG |
| | | | | GAACTCAGGCGCCCTGACCAGCGGCGTGCACACC |
| | | | | TTCCCGGCTGTCCTACAGTCCTCAGGACTCTACT |
| | | | | CCCTCAGCAGCGTGGTGACCGTGCCCTCCAGCAG |
| | | | | CTTGGGCACCCAGACCTACATCTGCAACGTGAAT |
| | | | | CACAAGCCCAGCAACACCAAGGTGGACAAGAAAG |
| | | | | TTGAGCCCAAATCTTGTGACAAAACTCACACATG |
| | | | | CCCACCGTGCCCAGCACCTGAACTCCTGGGGGGA |
| | | | | CCGTCAGTCTTCCTCTTCCCCCCAAAACCCAAGG |
| | | | | ACACCCTCATGATCTCCCGGACCCCTGAGGTCAC |
| | | | | ATGCGTGGTGGTGGACGTGAGCCACGAAGACCCT |
| | | | | GAGGTCAAGTTCAACTGGTACGTGGACGGCGTGG |
| | | | | AGGTGCATAATGCCAAGACAAAGCCGCGGGAGGA |
| | | | | GCAGTACAACAGCACGTACCGTGTGGTCAGCGTC |
| | | | | CTCACCGTCCTGCACCAGGACTGGCTGAATGGCA |
| | | | | AGGAGTACAAGTGCAAGGTCTCCAACAAAGCCCT |
| | | | | CCCAGCCCCCATCGAGAAAACCATCTCCAAAGCC |
| | | | | AAAGGGCAGCCCCGAGAACCACAGGTGTACACCC |
| | | | | TGCCCCCATCCCGGGATGAGCTGACCAAGAACCA |
| | | | | GGTCAGCCTGACCTGCCTGGTCAAAGGCTTCTAT |
| | | | | CCCAGCGACATCGCCGTGGAGTGGGAGAGCAATG |
| | | | | GGCAGCCGGAGAACAACTACAAGACCACGCCTCC |
| | | | | CGTGCTGGACTCCGACGGCTCCTTCTTCTTATAT |
| | | | | TCAAAGCTCACCGTGGACAAGAGCAGGTGGCAGC |
| | | | | AGGGGAACGTCTTCTCATGCTCCGTGATGCATGA |
| | | | | GGCTCTGCACAACCACTACACGCAGAAGAGCCTC |
| | | | | TCCCTGTCTCCCGGGAAA |
| 23P11 | HC | H1-14AAS | 204 | GAAGTGCAGCTGGTTGAATCTGGCGGAGGACTGA |
| | | | | TTCAGCCTGGCGGCTCTCTGAGACTGTCTTGTGC |
| | | | | CGCCTCTGGCTTCAGCCTGAGCAACTACTACATC |
| | | | | AACTGGGTCCGACAGGCCCCTGGCAAAGGACTGG |
| | | | | AATGGATCGGCTTCATCCCTATGTACGGCACCAC |
| | | | | ACACTACGCCAACTGGGCCAAGGGCAGATTCACC |
| | | | | ATCAGCCGGGACAACAGCAAGAACACCCTGTACC |
| | | | | TGCAGATGAACAGCCTGAGAGCCGAGGACACCGC |
| | | | | CGTGTACTATTGTGCCAGAGGAATCGCCAGCATG |
| | | | | TTCTACCCTTCTATCTGGGGCCAGGGCACCCTGG |
| | | | | TCACAGTTAGITCTGCATCCACCAAGGGCCCATC |
| | | | | GGTCTTCCCCCTGGCACCCTCCTCCAAGAGCACC |
| | | | | TCTGGGGGCACAGCGGCCCTGGGCTGCCTGGTCA |
| | | | | AGGACTACTTCCCCGAACCGGTGACGGTGTCGTG |
| | | | | GAACTCAGGCGCCCTGACCAGCGGCGTGCACACC |
| | | | | TTCCCGGCTGTCCTACAGTCCTCAGGACTCTACT |
| | | | | CCCTCAGCAGCGTGGTGACCGTGCCCTCCAGCAG |
| | | | | CTTGGGCACCCAGACCTACATCTGCAACGTGAAT |
| | | | | CACAAGCCCAGCAACACCAAGGTGGACAAGAAAG |
| | | | | TTGAGCCCAAATCTTGTGACAAAACTCACACATG |
| | | | | CCCACCGTGCCCAGCACCTGAGGCCGCCGGGGGA |
| | | | | TCCTCAGTCTTCCTGTTCCCCCCAAAACCCAAGG |
| | | | | ACACTCTCATGATCTCCCGGACCCCTGAGGTCAC |
| | | | | GTGCGTGGTGGTGGACGTGAGCCAGGAAGACCCC |
| | | | | GAGGTCCAGTTCAACTGGTACGTGGATGGCGTGG |
| | | | | AGGTGCATAATGCCAAGACAAAGCCGCGGGAGGA |
| | | | | GCAGTACAACAGCACGTACCGTGTGGTCAGCGTC |
| | | | | CTCACCGTCCTGCACCAGGACTGGCTGAACGGCA |
| | | | | AGGAGTACAAGTGCAAGGTCTCCAACAAAGCCCT |
| | | | | CCCGGCCCCCATCGAGAAAACCATCTCCAAAGCC |
| | | | | AAAGGGCAGCCCCGAGAACCACAGGTGTACACCC |
| | | | | TGCCCCCATCCCGGGATGAGCTGACCAAGAACCA |
| | | | | GGTCAGCCTGACCTGCCTGGTCAAAGGCTTCTAT |
| | | | | CCCAGCGACATCGCCGTGGAGTGGGAGAGCAATG |
| | | | | GGCAGCCGGAGAACAACTACAAGACCACGCCTCC |
| | | | | CGTGCTGGACTCCGACGGCTCCTTCTTCTTATAT |
| | | | | TCAAAGCTCACCGTGGACAAGAGCAGGTGGCAGC |
| | | | | AGGGGAACGTCTTCTCATGCTCCGTGATGCATGA |
| | | | | GGCTCTGCACAACCACTACACGCAGAAGAGCCTC |
| | | | | TCCCTGTCTCCCGGGAAA |
| 23P11 | HC | H1-14AAS- | 205 | GAAGTGCAGCTGGTTGAATCTGGCGGAGGACTGA |
| | | A118C | | TTCAGCCTGGCGGCTCTCTGAGACTGTCTTGTGC |
| | | | | CGCCTCTGGCTTCAGCCTGAGCAACTACTACATC |
| | | | | AACTGGGTCCGACAGGCCCCTGGCAAAGGACTGG |
| | | | | AATGGATCGGCTTCATCCCTATGTACGGCACCAC |
| | | | | ACACTACGCCAACTGGGCCAAGGGCAGATTCACC |
| | | | | ATCAGCCGGGACAACAGCAAGAACACCCTGTACC |
| | | | | TGCAGATGAACAGCCTGAGAGCCGAGGACACCGC |
| | | | | CGTGTACTATTGTGCCAGAGGAATCGCCAGCATG |
| | | | | TTCTACCCTTCTATCTGGGGCCAGGGCACCCTGG |
| | | | | TCACAGTTAGTTCTTGCTCCACCAAGGGCCCATC |
| | | | | GGTCTTCCCCCTGGCACCCTCCTCCAAGAGCACC |
| | | | | TCTGGGGGCACAGCGGCCCTGGGCTGCCTGGTCA |
| | | | | AGGACTACTTCCCCGAACCGGTGACGGTGTCGTG |
| | | | | GAACTCAGGCGCCCTGACCAGCGGCGTGCACACC |
| | | | | TTCCCGGCTGTCCTACAGTCCTCAGGACTCTACT |
| | | | | CCCTCAGCAGCGTGGTGACCGTGCCCTCCAGCAG |
| | | | | CTTGGGCACCCAGACCTACATCTGCAACGTGAAT |
| | | | | CACAAGCCCAGCAACACCAAGGTGGACAAGAAAG |
| | | | | TTGAGCCCAAATCTTGTGACAAAACTCACACATG |
| | | | | CCCACCGTGCCCAGCACCTGAGGCCGCCGGGGGA |
| | | | | TCCTCAGTCTTCCTGTTCCCCCCAAAACCCAAGG |
| | | | | ACACTCTCATGATCTCCCGGACCCCTGAGGTCAC |
| | | | | GTGCGTGGTGGTGGACGTGAGCCAGGAAGACCCC |
| | | | | GAGGTCCAGTTCAACTGGTACGTGGATGGCGTGG |
| | | | | AGGTGCATAATGCCAAGACAAAGCCGCGGGAGGA |
| | | | | GCAGTACAACAGCACGTACCGTGTGGTCAGCGTC |
| | | | | CTCACCGTCCTGCACCAGGACTGGCTGAACGGCA |
| | | | | AGGAGTACAAGTGCAAGGTCTCCAACAAAGCCCT |
| | | | | CCCGGCCCCCATCGAGAAAACCATCTCCAAAGCC |
| | | | | AAAGGGCAGCCCCGAGAACCACAGGTGTACACCC |
| | | | | TGCCCCCATCCCGGGATGAGCTGACCAAGAACCA |
| | | | | GGTCAGCCTGACCTGCCTGGTCAAAGGCTTCTAT |
| | | | | CCCAGCGACATCGCCGTGGAGTGGGAGAGCAATG |
| | | | | GGCAGCCGGAGAACAACTACAAGACCACGCCTCC |
| | | | | CGTGCTGGACTCCGACGGCTCCTTCTTCTTATAT |
| | | | | TCAAAGCTCACCGTGGACAAGAGCAGGTGGCAGC |
| | | | | AGGGGAACGTCTTCTCATGCTCCGTGATGCATGA |
| | | | | GGCTCTGCACAACCACTACACGCAGAAGAGCCTC |
| | | | | TCCCTGTCTCCCGGGAAA |
| 23P11 | LC | L1 | 206 | GACGTTGTGATGACACAGAGCCCTAGCAGCCTGA |
| | | | | GCGCCTCTGTGGGCGATAGAGTGACAATCACCTG |
| | | | | TCAGGCCAGCGAGGACATCCACGGCTATCTGGCC |
| | | | | TGGTATCAGCAGAAGCCTGGAAAGGCCCCTAAGC |
| | | | | TGCTGATCTACTACGCCTCTACACTGGCCAGCGG |
| | | | | CGTGCCAAGCAGATTTTCTGGCTCTGGCAGCGGC |
| | | | | ACCGACTTCACCCTGACAATTTCTAGCCTCCAGC |
| | | | | CTGAGGACTTCGCCACCTACTATTGTCTCGGAGA |
| | | | | TGGCAGCACCAGCGGCATCACATTTGGCCAGGGC |
| | | | | ACCAAGGTGGAAATCAAAAGAACAGTGGCCGCTC |
| | | | | CGAGCGTGTTCATCTTTCCACCAAGCGACGAGCA |
| | | | | GCTGAAAAGCGGCACAGCCTCTGTCGTGTGCCTG |
| | | | | CTGAACAACTTCTACCCCAGAGAAGCCAAGGTGC |
| | | | | AGTGGAAGGTGGACAATGCCCTGCAGAGCGGCAA |
| | | | | TAGCCAAGAGAGCGTGACCGAGCAGGACAGCAAG |
| | | | | GATAGCACCTACAGCCTGTCCAGCACACTGACCC |
| | | | | TGAGCAAGGCCGACTACGAGAAGCACAAAGTGTA |
| | | | | CGCCTGCGAAGTGACACACCAGGGCCTGTCTAGC |
| | | | | CCTGTGACCAAGAGCTTCAACCGGGGCGAGTGT |
| 23P11 | LC | L1-C80 | 207 | GACGTTGTGATGACACAGAGCCCTAGCAGCCTGA |
| | | | | GCGCCTCTGTGGGCGATAGAGTGACAATCACCTG |
| | | | | TCAGGCCAGCGAGGACATCCACGGCTATCTGGCC |
| | | | | TGGTATCAGCAGAAGCCTGGAAAGGCCCCTAAGC |
| | | | | TGCTGATCTACTACGCCTCTACACTGGCCAGCGG |
| | | | | CGTGCCAAGCAGATTTTCTGGCTCTGGCAGCGGC |
| | | | | ACCGACTTCACCCTGACAATTTCTAGCCTCCAGT |
| | | | | GCGAGGACGTGGCCACCTACTATTGTCTCGGAGA |
| | | | | TGGCAGCACCAGCGGCATCACATTTGGCCAGGGC |
| | | | | ACCAAGGTGGAAATCAAAAGAACAGTGGCCGCTC |
| | | | | CGAGCGTGTTCATCTTTCCACCAAGCGACGAGCA |
| | | | | GCTGAAAAGCGGCACAGCCTCTGTCGTGTGCCTG |
| | | | | CTGAACAACTTCTACCCCAGAGAAGCCAAGGTGC |
| | | | | AGTGGAAGGTGGACAATGCCCTGCAGAGCGGCAA |
| | | | | TAGCCAAGAGAGCGTGACCGAGCAGGACAGCAAG |
| | | | | GATAGCACCTACAGCCTGTCCAGCACACTGACCC |
| | | | | TGAGCAAGGCCGACTACGAGAAGCACAAAGTGTA |
| | | | | CGCCTGCGAAGTGACACACCAGGGCCTGTCTAGC |
| | | | | CCTGTGACCAAGAGCTTCAACCGGGGCGAGTGT |
| 2406 | HC | H1 | 208 | GAAGTGCAGCTGGTTGAATCTGGCGGAGGACTGA |
| | | | | TTCAGCCTGGCGGCTCTCTGAGACTGTCTTGTGC |
| | | | | CGCCTCTGGCATCGATCTGAGCAGCTCTACAATG |
| | | | | GCCTGGGTTCGACAGGCCCCTGGCAAAGGACTGG |
| | | | | AATGGATCGGCATCATTGCCAGCAGCGGCTCTGC |
| | | | | CTATTATGCCGGATGGGCCAAGGGCAGATTCACC |
| | | | | ATCAGCCGGGACAACAGCAAGAACACCCTGTACC |
| | | | | TGCAGATGAACAGCCTGAGAGCCGAGGACACCGC |
| | | | | CGTGTACTATTGTGTTAGACAGGTGCCCGGCTAC |
| | | | | AACGACAACAGATATGTGTGGGGCCAGGGCACCC |
| | | | | TGGTCACAGTTAGTTCTGCATCCACCAAGGGCCC |
| | | | | ATCGGTCTTCCCCCTGGCACCCTCCTCCAAGAGC |
| | | | | ACCTCTGGGGGCACAGCGGCCCTGGGCTGCCTGG |
| | | | | TCAAGGACTACTTCCCCGAACCGGTGACGGTGTC |
| | | | | GTGGAACTCAGGCGCCCTGACCAGCGGCGTGCAC |
| | | | | ACCTTCCCGGCTGTCCTACAGTCCTCAGGACTCT |
| | | | | ACTCCCTCAGCAGCGTGGTGACCGTGCCCTCCAG |
| | | | | CAGCTTGGGCACCCAGACCTACATCTGCAACGTG |
| | | | | AATCACAAGCCCAGCAACACCAAGGTGGACAAGA |
| | | | | AAGTTGAGCCCAAATCTTGTGACAAAACTCACAC |
| | | | | ATGCCCACCGTGCCCAGCACCTGAACTCCTGGGG |
| | | | | GGACCGTCAGTCTTCCTCTTCCCCCCAAAACCCA |
| | | | | AGGACACCCTCATGATCTCCCGGACCCCTGAGGT |
| | | | | CACATGCGTGGTGGTGGACGTGAGCCACGAAGAC |
| | | | | CCTGAGGTCAAGTTCAACTGGTACGTGGACGGCG |
| | | | | TGGAGGTGCATAATGCCAAGACAAAGCCGCGGGA |
| | | | | GGAGCAGTACAACAGCACGTACCGTGTGGTCAGC |
| | | | | GTCCTCACCGTCCTGCACCAGGACTGGCTGAATG |
| | | | | GCAAGGAGTACAAGTGCAAGGTCTCCAACAAAGC |
| | | | | CCTCCCAGCCCCCATCGAGAAAACCATCTCCAAA |
| | | | | GCCAAAGGGCAGCCCCGAGAACCACAGGTGTACA |
| | | | | CCCTGCCCCCATCCCGGGATGAGCTGACCAAGAA |
| | | | | CCAGGTCAGCCTGACCTGCCTGGTCAAAGGCTTC |
| | | | | TATCCCAGCGACATCGCCGTGGAGTGGGAGAGCA |
| | | | | ATGGGCAGCCGGAGAACAACTACAAGACCACGCC |
| | | | | TCCCGTGCTGGACTCCGACGGCTCCTTCTTCTTA |
| | | | | TATTCAAAGCTCACCGTGGACAAGAGCAGGTGGC |
| | | | | AGCAGGGGAACGTCTTCTCATGCTCCGTGATGCA |
| | | | | TGAGGCTCTGCACAACCACTACACGCAGAAGAGC |
| | | | | CTCTCCCTGTCTCCCGGGAAA |
| 2406 | HC | H1-A118C | 209 | GAAGTGCAGCTGGTTGAATCTGGCGGAGGACTGA |
| | | | | TTCAGCCTGGCGGCTCTCTGAGACTGTCTTGTGC |
| | | | | CGCCTCTGGCATCGATCTGAGCAGCTCTACAATG |
| | | | | GCCTGGGTTCGACAGGCCCCTGGCAAAGGACTGG |
| | | | | AATGGATCGGCATCATTGCCAGCAGCGGCTCTGC |
| | | | | CTATTATGCCGGATGGGCCAAGGGCAGATTCACC |
| | | | | ATCAGCCGGGACAACAGCAAGAACACCCTGTACC |
| | | | | TGCAGATGAACAGCCTGAGAGCCGAGGACACCGC |
| | | | | CGTGTACTATTGTGTTAGACAGGTGCCCGGCTAC |
| | | | | AACGACAACAGATATGTGTGGGGCCAGGGCACCC |
| | | | | TGGTCACAGTTAGTTCTTGCTCCACCAAGGGCCC |
| | | | | ATCGGTCTTCCCCCTGGCACCCTCCTCCAAGAGC |
| | | | | ACCTCTGGGGGCACAGCGGCCCTGGGCTGCCTGG |
| | | | | TCAAGGACTACTTCCCCGAACCGGTGACGGTGTC |
| | | | | GTGGAACTCAGGCGCCCTGACCAGCGGCGTGCAC |
| | | | | ACCTTCCCGGCTGTCCTACAGTCCTCAGGACTCT |
| | | | | ACTCCCTCAGCAGCGTGGTGACCGTGCCCTCCAG |
| | | | | CAGCTTGGGCACCCAGACCTACATCTGCAACGTG |
| | | | | AATCACAAGCCCAGCAACACCAAGGTGGACAAGA |
| | | | | AAGTTGAGCCCAAATCTTGTGACAAAACTCACAC |
| | | | | ATGCCCACCGTGCCCAGCACCTGAACTCCTGGGG |
| | | | | GGACCGTCAGTCTTCCTCTTCCCCCCAAAACCCA |
| | | | | AGGACACCCTCATGATCTCCCGGACCCCTGAGGT |
| | | | | CACATGCGTGGTGGTGGACGTGAGCCACGAAGAC |
| | | | | CCTGAGGTCAAGTTCAACTGGTACGTGGACGGCG |
| | | | | TGGAGGTGCATAATGCCAAGACAAAGCCGCGGGA |
| | | | | GGAGCAGTACAACAGCACGTACCGTGTGGTCAGC |
| | | | | GTCCTCACCGTCCTGCACCAGGACTGGCTGAATG |
| | | | | GCAAGGAGTACAAGTGCAAGGTCTCCAACAAAGC |
| | | | | CCTCCCAGCCCCCATCGAGAAAACCATCTCCAAA |
| | | | | GCCAAAGGGCAGCCCCGAGAACCACAGGTGTACA |
| | | | | CCCTGCCCCCATCCCGGGATGAGCTGACCAAGAA |
| | | | | CCAGGTCAGCCTGACCTGCCTGGTCAAAGGCTTC |
| | | | | TATCCCAGCGACATCGCCGTGGAGTGGGAGAGCA |
| | | | | ATGGGCAGCCGGAGAACAACTACAAGACCACGCC |
| | | | | TCCCGTGCTGGACTCCGACGGCTCCTTCTTCTTA |
| | | | | TATTCAAAGCTCACCGTGGACAAGAGCAGGTGGC |
| | | | | AGCAGGGGAACGTCTTCTCATGCTCCGTGATGCA |
| | | | | TGAGGCTCTGCACAACCACTACACGCAGAAGAGC |
| | | | | CTCTCCCTGTCTCCCGGGAAA |
| 2406 | HC | H1-14AAS | 210 | GAAGTGCAGCTGGTTGAATCTGGCGGAGGACTGA |
| | | | | TTCAGCCTGGCGGCTCTCTGAGACTGTCTTGTGC |
| | | | | CGCCTCTGGCATCGATCTGAGCAGCTCTACAATG |
| | | | | GCCTGGGTTCGACAGGCCCCTGGCAAAGGACTGG |
| | | | | AATGGATCGGCATCATTGCCAGCAGCGGCTCTGC |
| | | | | CTATTATGCCGGATGGGCCAAGGGCAGATTCACC |
| | | | | ATCAGCCGGGACAACAGCAAGAACACCCTGTACC |
| | | | | TGCAGATGAACAGCCTGAGAGCCGAGGACACCGC |
| | | | | CGTGTACTATTGTGTTAGACAGGTGCCCGGCTAC |
| | | | | AACGACAACAGATATGTGTGGGGCCAGGGCACCC |
| | | | | TGGTCACAGTTAGTTCTGCATCCACCAAGGGCCC |
| | | | | ATCGGTCTTCCCCCTGGCACCCTCCTCCAAGAGC |
| | | | | ACCTCTGGGGGCACAGCGGCCCTGGGCTGCCTGG |
| | | | | TCAAGGACTACTTCCCCGAACCGGTGACGGTGTC |
| | | | | GTGGAACTCAGGCGCCCTGACCAGCGGCGTGCAC |
| | | | | ACCTTCCCGGCTGTCCTACAGTCCTCAGGACTCT |
| | | | | ACTCCCTCAGCAGCGTGGTGACCGTGCCCTCCAG |
| | | | | CAGCTTGGGCACCCAGACCTACATCTGCAACGTG |
| | | | | AATCACAAGCCCAGCAACACCAAGGTGGACAAGA |
| | | | | AAGTTGAGCCCAAATCTTGTGACAAAACTCACAC |
| | | | | ATGCCCACCGTGCCCAGCACCTGAGGCCGCCGGG |
| | | | | GGATCCTCAGTCTTCCTGTTCCCCCCAAAACCCA |
| | | | | AGGACACTCTCATGATCTCCCGGACCCCTGAGGT |
| | | | | CACGTGCGTGGTGGTGGACGTGAGCCAGGAAGAC |
| | | | | CCCGAGGTCCAGTTCAACTGGTACGTGGATGGCG |
| | | | | TGGAGGTGCATAATGCCAAGACAAAGCCGCGGGA |
| | | | | GGAGCAGTACAACAGCACGTACCGTGTGGTCAGC |
| | | | | GTCCTCACCGTCCTGCACCAGGACTGGCTGAACG |
| | | | | GCAAGGAGTACAAGTGCAAGGTCTCCAACAAAGC |
| | | | | CCTCCCGGCCCCCATCGAGAAAACCATCTCCAAA |
| | | | | GCCAAAGGGCAGCCCCGAGAACCACAGGTGTACA |
| | | | | CCCTGCCCCCATCCCGGGATGAGCTGACCAAGAA |
| | | | | CCAGGTCAGCCTGACCTGCCTGGTCAAAGGCTTC |
| | | | | TATCCCAGCGACATCGCCGTGGAGTGGGAGAGCA |
| | | | | ATGGGCAGCCGGAGAACAACTACAAGACCACGCC |
| | | | | TCCCGTGCTGGACTCCGACGGCTCCTTCTTCTTA |
| | | | | TATTCAAAGCTCACCGTGGACAAGAGCAGGTGGC |
| | | | | AGCAGGGGAACGTCTTCTCATGCTCCGTGATGCA |
| | | | | TGAGGCTCTGCACAACCACTACACGCAGAAGAGC |
| | | | | CTCTCCCTGTCTCCCGGGAAA |
| 2406 | HC | H1-14AAS- | 211 | GAAGTGCAGCTGGTTGAATCTGGCGGAGGACTGA |
| | | A118C | | TTCAGCCTGGCGGCTCTCTGAGACTGTCTTGTGC |
| | | | | CGCCTCTGGCATCGATCTGAGCAGCTCTACAATG |
| | | | | GCCTGGGTTCGACAGGCCCCTGGCAAAGGACTGG |
| | | | | AATGGATCGGCATCATTGCCAGCAGCGGCTCTGC |
| | | | | CTATTATGCCGGATGGGCCAAGGGCAGATTCACC |
| | | | | ATCAGCCGGGACAACAGCAAGAACACCCTGTACC |
| | | | | TGCAGATGAACAGCCTGAGAGCCGAGGACACCGC |
| | | | | CGTGTACTATTGTGTTAGACAGGTGCCCGGCTAC |
| | | | | AACGACAACAGATATGTGTGGGGCCAGGGCACCC |
| | | | | TGGTCACAGTTAGTTCTTGCTCCACCAAGGGCCC |
| | | | | ATCGGTCTTCCCCCTGGCACCCTCCTCCAAGAGC |
| | | | | ACCTCTGGGGGCACAGCGGCCCTGGGCTGCCTGG |
| | | | | TCAAGGACTACTTCCCCGAACCGGTGACGGTGTC |
| | | | | GTGGAACTCAGGCGCCCTGACCAGCGGCGTGCAC |
| | | | | ACCTTCCCGGCTGTCCTACAGTCCTCAGGACTCT |
| | | | | ACTCCCTCAGCAGCGTGGTGACCGTGCCCTCCAG |
| | | | | CAGCTTGGGCACCCAGACCTACATCTGCAACGTG |
| | | | | AATCACAAGCCCAGCAACACCAAGGTGGACAAGA |
| | | | | AAGTTGAGCCCAAATCTTGTGACAAAACTCACAC |
| | | | | ATGCCCACCGTGCCCAGCACCTGAGGCCGCCGGG |
| | | | | GGATCCTCAGTCTTCCTGTTCCCCCCAAAACCCA |
| | | | | AGGACACTCTCATGATCTCCCGGACCCCTGAGGT |
| | | | | CACGTGCGTGGTGGTGGACGTGAGCCAGGAAGAC |
| | | | | CCCGAGGTCCAGTTCAACTGGTACGTGGATGGCG |
| | | | | TGGAGGTGCATAATGCCAAGACAAAGCCGCGGGA |
| | | | | GGAGCAGTACAACAGCACGTACCGTGTGGTCAGC |
| | | | | GTCCTCACCGTCCTGCACCAGGACTGGCTGAACG |
| | | | | GCAAGGAGTACAAGTGCAAGGTCTCCAACAAAGC |
| | | | | CCTCCCGGCCCCCATCGAGAAAACCATCTCCAAA |
| | | | | GCCAAAGGGCAGCCCCGAGAACCACAGGTGTACA |
| | | | | CCCTGCCCCCATCCCGGGATGAGCTGACCAAGAA |
| | | | | CCAGGTCAGCCTGACCTGCCTGGTCAAAGGCTTC |
| | | | | TATCCCAGCGACATCGCCGTGGAGTGGGAGAGCA |
| | | | | ATGGGCAGCCGGAGAACAACTACAAGACCACGCC |
| | | | | TCCCGTGCTGGACTCCGACGGCTCCTTCTTCTTA |
| | | | | TATTCAAAGCTCACCGTGGACAAGAGCAGGTGGC |
| | | | | AGCAGGGGAACGTCTTCTCATGCTCCGTGATGCA |
| | | | | TGAGGCTCTGCACAACCACTACACGCAGAAGAGC |
| | | | | CTCTCCCTGTCTCCCGGGAAA |
| 2406 | LC | L1 | 212 | GCTATTGTGATGACACAGAGCCCTAGCAGCCTGA |
| | | | | GCGCCTCTGTGGGCGATAGAGTGACAATCACCTG |
| | | | | TCAGGCCAGCGAGAGCGTGTACAACGCCAACAGC |
| | | | | TGTAGCTGGTATCAGCAGAAGCCTGGCAAGGCCC |
| | | | | CTAAGCTGCTGATCTACGATGCCTCTGATCTGGC |
| | | | | CAGCGGCGTGCCAAGCAGATTTTCTGGCTCTGGC |
| | | | | AGCGGCACCGACTTCACCCTGACAATTTCTAGCC |
| | | | | TCCAGCCTGAGGACTTCGCCACCTACTATTGTGC |
| | | | | CGGCTACAAAGGCAATGGCGACGCCGCTTTTGGC |
| | | | | CAGGGCACAAAGGTGGAAATCAAAAGAACAGTGG |
| | | | | CCGCTCCGAGCGTGTTCATCTTTCCACCAAGCGA |
| | | | | CGAGCAGCTGAAAAGCGGCACAGCCTCTGTCGTG |
| | | | | TGCCTGCTGAACAACTTCTACCCCAGAGAAGCCA |
| | | | | AGGTGCAGTGGAAGGTGGACAATGCCCTGCAGAG |
| | | | | CGGCAATAGCCAAGAGAGCGTGACCGAGCAGGAC |
| | | | | AGCAAGGATAGCACCTACAGCCTGTCCAGCACAC |
| | | | | TGACCCTGAGCAAGGCCGACTACGAGAAGCACAA |
| | | | | AGTGTACGCCTGCGAAGTGACACACCAGGGCCTG |
| | | | | TCTAGCCCTGTGACCAAGAGCTTCAACCGGGGCG |
| | | | | AGTGT |
| 2406 | LC | L1-C80 | 213 | GCTATTGTGATGACACAGAGCCCTAGCAGCCTGA |
| | | | | GCGCCTCTGTGGGCGATAGAGTGACAATCACCTG |
| | | | | TCAGGCCAGCGAGAGCGTGTACAACGCCAACAGC |
| | | | | TGTAGCTGGTATCAGCAGAAGCCTGGCAAGGCCC |
| | | | | CTAAGCTGCTGATCTACGATGCCTCTGATCTGGC |
| | | | | CAGCGGCGTGCCAAGCAGATTTTCTGGCTCTGGC |
| | | | | AGCGGCACCGACTTCACCCTGACAATTTCTAGCC |
| | | | | TCCAGTGCGAGGACGTGGCCACCTACTATTGTGC |
| | | | | CGGCTACAAAGGCAATGGCGACGCCGCTTTTGGC |
| | | | | CAGGGCACAAAGGTGGAAATCAAAAGAACAGTGG |
| | | | | CCGCTCCGAGCGTGTTCATCTTTCCACCAAGCGA |
| | | | | CGAGCAGCTGAAAAGCGGCACAGCCTCTGTCGTG |
| | | | | TGCCTGCTGAACAACTTCTACCCCAGAGAAGCCA |
| | | | | AGGTGCAGTGGAAGGTGGACAATGCCCTGCAGAG |
| | | | | CGGCAATAGCCAAGAGAGCGTGACCGAGCAGGAC |
| | | | | AGCAAGGATAGCACCTACAGCCTGTCCAGCACAC |
| | | | | TGACCCTGAGCAAGGCCGACTACGAGAAGCACAA |
| | | | | AGTGTACGCCTGCGAAGTGACACACCAGGGCCTG |
| | | | | TCTAGCCCTGTGACCAAGAGCTTCAACCGGGGCG |
| | | | | AGTGT |

In some embodiments, the sequences of the heavy chain variable domains, light chain variable domains, full-length heavy chains, and full-length light chains may be “mixed and matched” to create variants of the anti-CD73 antibodies, e.g., heavy and light chains from any of the 190K12, 366F5, 23P11, or 24O6 variants. In some embodiments, only mixing and matching from among variants of a particular starting clone are used (e.g., a heavy chain variant of 190K12 may be paired with a light chain variant of 190K12). Such “mixed and matched” anti-CD73 antibodies can be tested using binding assays known in the art (e.g., FACS titration and other assays described in the Examples). For example, an amino acid sequence corresponding to a light chain variable domain from a particular set of light chain variable domains listed above in Table 6 for a specific antibody may be replaced with another amino acid sequence corresponding to another light chain variable domain option from the table. For example, the amino acid sequence of SEQ ID NO: 50 may be replaced with the amino acid sequence of SEQ ID NO: 51 to create a variant of 190K12 that comprises engineered cysteines at position 80 in the Vx region (C80) to generate site-specific DAR 2 ADCs. The same applies to the mixing and matching of full-length heavy chains and full-length light chains.

In various embodiments, the antibodies disclosed herein may comprise any set of heavy and light chain variable domains listed in the tables above (e.g., 190K12 heavy and light chain variable domains, 366F5 heavy and light chain variable domains, 23P11 heavy and light chain variable domains, or 24O6 heavy and light chain variable domains), or the set of six CDR sequences from the heavy and light chain set (e.g., three 190K12 heavy chain CDRs and three 190K12 light chain CDRs, three 366F5 heavy chain CDRs and three 366F5 light chain CDRs, three 23P11 heavy chain CDRs and three 23P11 light chain CDRs, or three 24O6 heavy chain CDRs and three 24O6 light chain CDRs). In some embodiments, the antibodies further comprise human heavy and light chain constant domains or fragments thereof. In various embodiments, the antibodies may comprise any set of full-length heavy chain and full-length light chain sequences listed in the tables above (e.g., 190K12 full-length heavy and light chains, 366F5 full-length heavy and light chains, 23P11 full-length heavy and light chains, or 24O6 full-length heavy and light chains). In some embodiments, the antibodies may comprise a human IgG heavy chain constant domain and a human kappa light chain constant domain. In some embodiments, the antibodies may comprise a human IgG1, IgG2, IgG3, or IgG4 heavy chain constant domain. In various embodiments, an antibody of the present invention comprises a human immunoglobulin G subtype 1 (IgG1) heavy chain constant domain with a human Ig kappa light chain constant domain. In some embodiments, the constant domain is a modified version of a human constant domain, e.g., comprising one or more of L234A, L235A, P238S, H268Q, and/or K274Q modifications of a human IgG1 heavy chain constant domain.

In some embodiments, an anti-CD73 antibody or antigen-binding fragment thereof provided herein comprises three heavy chain CDRs and three light chain CDRs as follows: heavy chain CDR1 (HCDR1) comprising SEQ ID NO: 1, heavy chain CDR2 (HCDR2) comprising SEQ ID NO: 2, heavy chain CDR3 (HCDR3) comprising SEQ ID NO: 3; light chain CDR1 (LCDR1) comprising SEQ ID NO: 4, light chain CDR2 (LCDR2) comprising SEQ ID NO: 5, and light chain CDR3 (LCDR3) comprising SEQ ID NO: 6, as defined by the Kabat numbering system

In some embodiments, an anti-CD73 antibody or antigen-binding fragment thereof provided herein comprises three heavy chain CDRs and three light chain CDRs as follows: HCDR1 comprising SEQ ID NO: 7, HCDR2 comprising SEQ ID NO: 8, HCDR3 comprising SEQ ID NO: 9; LCDR1 comprising SEQ ID NO: 10, LCDR2 comprising SEQ ID NO: 11, and LCDR3 comprising SEQ ID NO: 12, as defined by the IMGT numbering system.

In some embodiments, an anti-CD73 antibody or antigen-binding fragment thereof provided herein comprises three heavy chain CDRs and three light chain CDRs as follows: HCDR1 comprising SEQ ID NO: 13, HCDR2 comprising SEQ ID NO: 14, HCDR3 comprising SEQ ID NO: 15; LCDR1 comprising SEQ ID NO: 16, LCDR2 comprising SEQ ID NO: 17, and LCDR3 comprising SEQ ID NO: 18, as defined by the Kabat numbering system.

In some embodiments, an anti-CD73 antibody or antigen-binding fragment thereof provided herein comprises three heavy chain CDRs and three light chain CDRs as follows: HCDR1 comprising SEQ ID NO: 19, HCDR2 comprising SEQ ID NO: 20, HCDR3 comprising SEQ ID NO: 21; LCDR1 comprising SEQ ID NO: 22, LCDR2 comprising SEQ ID NO: 23, and LCDR3 comprising SEQ ID NO: 24, as defined by the IMGT numbering system.

In some embodiments, an anti-CD73 antibody or antigen-binding fragment thereof provided herein comprises three heavy chain CDRs and three light chain CDRs as follows: HCDR1 comprising SEQ ID NO: 25, HCDR2 comprising SEQ ID NO: 26, HCDR3 comprising SEQ ID NO: 27; LCDR1 comprising SEQ ID NO: 28, LCDR2 comprising SEQ ID NO: 29, and LCDR3 comprising SEQ ID NO: 30, as defined by the Kabat numbering system.

In some embodiments, an anti-CD73 antibody or antigen-binding fragment thereof provided herein comprises three heavy chain CDRs and three light chain CDRs as follows: HCDR1 comprising SEQ ID NO: 31, HCDR2 comprising SEQ ID NO: 32, HCDR3 comprising SEQ ID NO: 33; LCDR1 comprising SEQ ID NO: 34, LCDR2 comprising SEQ ID NO: 35, and LCDR3 comprising SEQ ID NO: 36, as defined by the IMGT numbering system.

In some embodiments, an anti-CD73 antibody or antigen-binding fragment thereof provided herein comprises three heavy chain CDRs and three light chain CDRs as follows: HCDR1 comprising SEQ ID NO: 37, HCDR2 comprising SEQ ID NO: 38, HCDR3 comprising SEQ ID NO: 39; LCDR1 comprising SEQ ID NO: 40, LCDR2 comprising SEQ ID NO: 41, and LCDR3 comprising SEQ ID NO: 42, as defined by the Kabat numbering system.

In some embodiments, an anti-CD73 antibody or antigen-binding fragment thereof provided herein comprises three heavy chain CDRs and three light chain CDRs as follows: HCDR1 comprising SEQ ID NO: 43, HCDR2 comprising SEQ ID NO: 44, HCDR3 comprising SEQ ID NO: 45; LCDR1 comprising SEQ ID NO: 46, LCDR2 comprising SEQ ID NO: 47, and LCDR3 comprising SEQ ID NO: 48, as defined by the IMGT numbering system.

In some embodiments, an anti-CD73 antibody provided herein comprises a heavy chain variable region comprising an amino acid sequence of SEQ ID NO: 49, and a light chain variable region comprising an amino acid sequence of SEQ ID NO: 50 or 51. In some embodiments, an anti-CD73 antibody provided herein comprises a heavy chain variable region comprising an amino acid sequence of SEQ ID NO: 52, and a light chain variable region comprising an amino acid sequence of SEQ ID NO: 53 or 54. In some embodiments, an anti-CD73 antibody provided herein comprises a heavy chain variable region comprising an amino acid sequence of SEQ ID NO: 55, and a light chain variable region comprising an amino acid sequence of SEQ ID NO: 56 or 57. In some embodiments, an anti-CD73 antibody provided herein comprises a heavy chain variable region comprising an amino acid sequence of SEQ ID NO: 58, and a light chain variable region comprising an amino acid sequence of SEQ ID NO: 59 or 60.

In some embodiments, an anti-CD73 antibody or antigen-binding fragment thereof provided herein comprises a heavy chain variable region comprising the amino acid sequence of SEQ ID NO: 49, and a light chain variable region comprising the amino acid sequence of SEQ ID NO: 50. In some embodiments, an anti-CD73 antibody or antigen-binding fragment thereof provided herein comprises a heavy chain variable region comprising the amino acid sequence of SEQ ID NO: 49, and a light chain variable region comprising an amino acid sequence of SEQ ID NO: 51.

In some embodiments, an anti-CD73 antibody or antigen-binding fragment thereof provided herein comprises a heavy chain variable region amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 49. In some embodiments, an anti-CD73 antibody or antigen-binding fragment thereof provided herein comprises a light chain variable region amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 50. In some embodiments, an anti-CD73 antibody or antigen-binding fragment thereof provided herein comprises a light chain variable region amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 51.

In some embodiments, an anti-CD73 antibody or antigen-binding fragment thereof provided herein comprises a heavy chain variable region comprising the amino acid sequence of SEQ ID NO: 52, and a light chain variable region comprising the amino acid sequence of SEQ ID NO: 53. In some embodiments, an anti-CD73 antibody or antigen-binding fragment thereof provided herein comprises a heavy chain variable region comprising the amino acid sequence of SEQ ID NO: 52, and a light chain variable region comprising an amino acid sequence of SEQ ID NO: 54.

In some embodiments, an anti-CD73 antibody or antigen-binding fragment thereof provided herein comprises a heavy chain variable region amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 52. In some embodiments, an anti-CD73 antibody or antigen-binding fragment thereof provided herein comprises a light chain variable region amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 53. In some embodiments, an anti-CD73 antibody or antigen-binding fragment thereof provided herein comprises a light chain variable region amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 54.

In some embodiments, an anti-CD73 antibody or antigen-binding fragment thereof provided herein comprises a heavy chain variable region comprising the amino acid sequence of SEQ ID NO: 55, and a light chain variable region comprising the amino acid sequence of SEQ ID NO: 56. In some embodiments, an anti-CD73 antibody or antigen-binding fragment thereof provided herein comprises a heavy chain variable region comprising the amino acid sequence of SEQ ID NO: 55, and a light chain variable region comprising an amino acid sequence of SEQ ID NO: 57.

In some embodiments, an anti-CD73 antibody or antigen-binding fragment thereof provided herein comprises a heavy chain variable region amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 55. In some embodiments, an anti-CD73 antibody or antigen-binding fragment thereof provided herein comprises a light chain variable region amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 56. In some embodiments, an anti-CD73 antibody or antigen-binding fragment thereof provided herein comprises a light chain variable region amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 57.

In some embodiments, an anti-CD73 antibody or antigen-binding fragment thereof provided herein comprises a heavy chain variable region comprising the amino acid sequence of SEQ ID NO: 58, and a light chain variable region comprising the amino acid sequence of SEQ ID NO: 59. In some embodiments, an anti-CD73 antibody or antigen-binding fragment thereof provided herein comprises a heavy chain variable region comprising the amino acid sequence of SEQ ID NO: 58, and a light chain variable region comprising an amino acid sequence of SEQ ID NO: 60.

In some embodiments, an anti-CD73 antibody or antigen-binding fragment thereof provided herein comprises a heavy chain variable region amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 58. In some embodiments, an anti-CD73 antibody or antigen-binding fragment thereof provided herein comprises a light chain variable region amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 59. In some embodiments, an anti-CD73 antibody or antigen-binding fragment thereof provided herein comprises a light chain variable region amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 60.

In various embodiments, any of the anti-CD73 antibodies disclosed herein may comprise a human IgG1 Fc domain. In some embodiments, an anti-CD73 antibody comprises a human IgG1 Fc domain that is modified to reduce binding to an FcγR as compared to an IgG1 Fc-containing antibody with a wild type IgG1 Fc domain. In some embodiments, the anti-CD73 antibodies comprise a mutated human IgG1 Fc domain that comprises one or more (e.g., all of) L234A, L235A, P238S, H268Q, and K274Q modifications to a human IgG1 heavy chain constant domain.

In various embodiments, the anti-CD73 antibodies comprise a human Ig kappa light chain constant region. In various embodiments, the anti-CD73 antibodies comprise a human Ig lambda light chain constant region.

In some embodiments, an anti-CD73 antibody provided herein comprises a heavy chain comprising an amino acid sequence of SEQ ID NO: 61, 62, 63, 64, 111, 112, 113, or 114 and a light chain comprising an amino acid sequence of SEQ ID NO: 65 or 66. In some embodiments, an anti-CD73 antibody provided herein comprises a heavy chain comprising an amino acid sequence of SEQ ID NO: 67, 68, 69, 70, 115, 116, 117, or 118 and a light chain comprising an amino acid sequence of SEQ ID NO: 71 or 72. In some embodiments, an anti-CD73 antibody provided herein comprises a heavy chain comprising an amino acid sequence of SEQ ID NO: 73, 74, 75, 76, 119, 120, 121, or 122 and a light chain comprising an amino acid sequence of SEQ ID NO: 77 or 78. In some embodiments, an anti-CD73 antibody provided herein comprises a heavy chain comprising an amino acid sequence of SEQ ID NO: 79, 80, 81, 82, 123, 124, 125, or 126 and a light chain comprising an amino acid sequence of SEQ ID NO: 83 or 84.

In some embodiments, an anti-CD73 antibody or antigen-binding fragment thereof provided herein comprises a heavy chain comprising an amino acid sequence of SEQ ID NO: 61 and a light chain amino acid sequence comprising an amino acid sequence of SEQ ID NO: 65. In some embodiments, an anti-CD73 antibody or antigen-binding fragment thereof provided herein comprises a heavy chain comprising an amino acid sequence of SEQ ID NO: 61 and a light chain comprising an amino acid sequence of SEQ ID NO: 66. In some embodiments, an anti-CD73 antibody or antigen-binding fragment thereof provided herein comprises a heavy chain comprising an amino acid sequence of SEQ ID NO: 62 and a light chain comprising an amino acid sequence of SEQ ID NO: 65. In some embodiments, an anti-CD73 antibody or antigen-binding fragment thereof provided herein comprises a heavy chain comprising an amino acid sequence of SEQ ID NO: 62 and a light chain comprising an amino acid sequence of SEQ ID NO: 66. In some embodiments, an anti-CD73 antibody or antigen-binding fragment thereof provided herein comprises a heavy chain comprising an amino acid sequence of SEQ ID NO: 63 and a light chain comprising an amino acid sequence of SEQ ID NO: 65. In some embodiments, an anti-CD73 antibody or antigen-binding fragment thereof provided herein comprises a heavy chain comprising an amino acid sequence of SEQ ID NO: 63 and a light chain comprising an amino acid sequence of SEQ ID NO: 66. In some embodiments, an anti-CD73 antibody or antigen-binding fragment thereof provided herein comprises a heavy chain comprising an amino acid sequence of SEQ ID NO: 64 and a light chain comprising an amino acid sequence of SEQ ID NO: 65. In some embodiments, an anti-CD73 antibody or antigen-binding fragment thereof provided herein comprises a heavy chain comprising an amino acid sequence of SEQ ID NO: 64 and a light chain comprising an amino acid sequence of SEQ ID NO: 66.

In some embodiments, an anti-CD73 antibody or antigen-binding fragment thereof provided herein comprises a heavy chain comprising an amino acid sequence of SEQ ID NO: 111 and a light chain amino acid sequence comprising an amino acid sequence of SEQ ID NO: 65. In some embodiments, an anti-CD73 antibody or antigen-binding fragment thereof provided herein comprises a heavy chain comprising an amino acid sequence of SEQ ID NO: 111 and a light chain comprising an amino acid sequence of SEQ ID NO: 66. In some embodiments, an anti-CD73 antibody or antigen-binding fragment thereof provided herein comprises a heavy chain comprising an amino acid sequence of SEQ ID NO: 112 and a light chain comprising an amino acid sequence of SEQ ID NO: 65. In some embodiments, an anti-CD73 antibody or antigen-binding fragment thereof provided herein comprises a heavy chain comprising an amino acid sequence of SEQ ID NO: 112 and a light chain comprising an amino acid sequence of SEQ ID NO: 66. In some embodiments, an anti-CD73 antibody or antigen-binding fragment thereof provided herein comprises a heavy chain comprising an amino acid sequence of SEQ ID NO: 113 and a light chain comprising an amino acid sequence of SEQ ID NO: 65. In some embodiments, an anti-CD73 antibody or antigen-binding fragment thereof provided herein comprises a heavy chain comprising an amino acid sequence of SEQ ID NO: 113 and a light chain comprising an amino acid sequence of SEQ ID NO: 66. In some embodiments, an anti-CD73 antibody or antigen-binding fragment thereof provided herein comprises a heavy chain comprising an amino acid sequence of SEQ ID NO: 114 and a light chain comprising an amino acid sequence of SEQ ID NO: 65. In some embodiments, an anti-CD73 antibody or antigen-binding fragment thereof provided herein comprises a heavy chain comprising an amino acid sequence of SEQ ID NO: 114 and a light chain comprising an amino acid sequence of SEQ ID NO: 66.

In some embodiments, an anti-CD73 antibody or antigen-binding fragment thereof provided herein comprises a heavy chain amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 61. In some embodiments, an anti-CD73 antibody or antigen-binding fragment thereof provided herein comprises a heavy chain amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 62. In some embodiments, an anti-CD73 antibody or antigen-binding fragment thereof provided herein comprises a heavy chain amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 63. In some embodiments, an anti-CD73 antibody or antigen-binding fragment thereof provided herein comprises a heavy chain amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 64. In some embodiments, an anti-CD73 antibody or antigen-binding fragment thereof provided herein comprises a light chain amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 65. In some embodiments, an anti-CD73 antibody or antigen-binding fragment thereof provided herein comprises a light chain amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 66.

In some embodiments, an anti-CD73 antibody or antigen-binding fragment thereof provided herein comprises a heavy chain amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 111. In some embodiments, an anti-CD73 antibody or antigen-binding fragment thereof provided herein comprises a heavy chain amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 112. In some embodiments, an anti-CD73 antibody or antigen-binding fragment thereof provided herein comprises a heavy chain amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 113. In some embodiments, an anti-CD73 antibody or antigen-binding fragment thereof provided herein comprises a heavy chain amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 114.

In some embodiments, the anti-CD73 antibody comprises a heavy chain comprising an amino acid sequence of SEQ ID NO: 63 and a light chain comprising an amino acid sequence of SEQ ID NO: 65. In some embodiments, the anti-CD73 antibody comprises a heavy chain comprising an amino acid sequence of SEQ ID NO: 63 and a light chain comprising an amino acid sequence of SEQ ID NO: 66. In some embodiments, the anti-CD73 antibody comprises a heavy chain comprising an amino acid sequence of SEQ ID NO: 64 and a light chain comprising an amino acid sequence of SEQ ID NO: 65. In some embodiments, the anti-CD73 antibody comprises a heavy chain comprising an amino acid sequence of SEQ ID NO: 64 and a light chain comprising an amino acid sequence of SEQ ID NO: 66.

In some embodiments, an anti-CD73 antibody or antigen-binding fragment thereof provided herein comprises a heavy chain comprising an amino acid sequence of SEQ ID NO: 67 and a light chain comprising an amino acid sequence of SEQ ID NO: 71. In some embodiments, an anti-CD73 antibody or antigen-binding fragment thereof provided herein comprises a heavy chain comprising an amino acid sequence of SEQ ID NO: 67 and a light chain comprising an amino acid sequence of SEQ ID NO: 72. In some embodiments, an anti-CD73 antibody or antigen-binding fragment thereof provided herein comprises a heavy chain comprising an amino acid sequence of SEQ ID NO: 68 and a light chain comprising an amino acid sequence of SEQ ID NO: 71. In some embodiments, an anti-CD73 antibody or antigen-binding fragment thereof provided herein comprises a heavy chain comprising an amino acid sequence of SEQ ID NO: 68 and a light chain comprising an amino acid sequence of SEQ ID NO: 72. In some embodiments, an anti-CD73 antibody or antigen-binding fragment thereof provided herein comprises a heavy chain comprising an amino acid sequence of SEQ ID NO: 69 and a light chain comprising an amino acid sequence of SEQ ID NO: 71. In some embodiments, an anti-CD73 antibody or antigen-binding fragment thereof provided herein comprises a heavy chain comprising an amino acid sequence of SEQ ID NO: 69 and a light chain comprising an amino acid sequence of SEQ ID NO: 72. In some embodiments, an anti-CD73 antibody or antigen-binding fragment thereof provided herein comprises a heavy chain comprising an amino acid sequence of SEQ ID NO: 70 and a light chain comprising an amino acid sequence of SEQ ID NO: 71. In some embodiments, an anti-CD73 antibody or antigen-binding fragment thereof provided herein comprises a heavy chain comprising an amino acid sequence of SEQ ID NO: 70 and a light chain comprising an amino acid sequence of SEQ ID NO: 72.

In some embodiments, an anti-CD73 antibody or antigen-binding fragment thereof provided herein comprises a heavy chain comprising an amino acid sequence of SEQ ID NO: 115 and a light chain comprising an amino acid sequence of SEQ ID NO: 71. In some embodiments, an anti-CD73 antibody or antigen-binding fragment thereof provided herein comprises a heavy chain comprising an amino acid sequence of SEQ ID NO: 115 and a light chain comprising an amino acid sequence of SEQ ID NO: 72. In some embodiments, an anti-CD73 antibody or antigen-binding fragment thereof provided herein comprises a heavy chain comprising an amino acid sequence of SEQ ID NO: 116 and a light chain comprising an amino acid sequence of SEQ ID NO: 71. In some embodiments, an anti-CD73 antibody or antigen-binding fragment thereof provided herein comprises a heavy chain comprising an amino acid sequence of SEQ ID NO: 116 and a light chain comprising an amino acid sequence of SEQ ID NO: 72. In some embodiments, an anti-CD73 antibody or antigen-binding fragment thereof provided herein comprises a heavy chain comprising an amino acid sequence of SEQ ID NO: 117 and a light chain comprising an amino acid sequence of SEQ ID NO: 71. In some embodiments, an anti-CD73 antibody or antigen-binding fragment thereof provided herein comprises a heavy chain comprising an amino acid sequence of SEQ ID NO: 117 and a light chain comprising an amino acid sequence of SEQ ID NO: 72. In some embodiments, an anti-CD73 antibody or antigen-binding fragment thereof provided herein comprises a heavy chain comprising an amino acid sequence of SEQ ID NO: 118 and a light chain comprising an amino acid sequence of SEQ ID NO: 71. In some embodiments, an anti-CD73 antibody or antigen-binding fragment thereof provided herein comprises a heavy chain comprising an amino acid sequence of SEQ ID NO: 118 and a light chain comprising an amino acid sequence of SEQ ID NO: 72.

In some embodiments, an anti-CD73 antibody or antigen-binding fragment thereof provided herein comprises a heavy chain amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 67. In some embodiments, an anti-CD73 antibody or antigen-binding fragment thereof provided herein comprises a heavy chain amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 68. In some embodiments, an anti-CD73 antibody or antigen-binding fragment thereof provided herein comprises a heavy chain amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 69. In some embodiments, an anti-CD73 antibody or antigen-binding fragment thereof provided herein comprises a heavy chain amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 70. In some embodiments, an anti-CD73 antibody or antigen-binding fragment thereof provided herein comprises a light chain amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 71. In some embodiments, an anti-CD73 antibody or antigen-binding fragment thereof provided herein comprises a light chain amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 72.

In some embodiments, an anti-CD73 antibody or antigen-binding fragment thereof provided herein comprises a heavy chain amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 115. In some embodiments, an anti-CD73 antibody or antigen-binding fragment thereof provided herein comprises a heavy chain amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 116. In some embodiments, an anti-CD73 antibody or antigen-binding fragment thereof provided herein comprises a heavy chain amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 117. In some embodiments, an anti-CD73 antibody or antigen-binding fragment thereof provided herein comprises a heavy chain amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 118.

In some embodiments, the anti-CD73 antibody comprises a heavy chain comprising an amino acid sequence of SEQ ID NO: 69 and a light chain comprising an amino acid sequence of SEQ ID NO: 71. In some embodiments, the anti-CD73 antibody comprises a heavy chain comprising an amino acid sequence of SEQ ID NO: 69 and a light chain comprising an amino acid sequence of SEQ ID NO: 72. In some embodiments, the anti-CD73 antibody comprises a heavy chain comprising an amino acid sequence of SEQ ID NO: 70 and a light chain comprising an amino acid sequence of SEQ ID NO: 71. In some embodiments, the anti-CD73 antibody comprises a heavy chain comprising an amino acid sequence of SEQ ID NO: 70 and a light chain comprising an amino acid sequence of SEQ ID NO: 72.

In some embodiments, an anti-CD73 antibody or antigen-binding fragment thereof provided herein comprises a heavy chain comprising an amino acid sequence of SEQ ID NO: 73 and a light chain comprising an amino acid sequence of SEQ ID NO: 77. In some embodiments, an anti-CD73 antibody or antigen-binding fragment thereof provided herein comprises a heavy chain comprising an amino acid sequence of SEQ ID NO: 73 and a light chain comprising an amino acid sequence of SEQ ID NO: 78. In some embodiments, an anti-CD73 antibody or antigen-binding fragment thereof provided herein comprises a heavy chain comprising an amino acid sequence of SEQ ID NO: 74 and a light chain comprising an amino acid sequence of SEQ ID NO: 77. In some embodiments, an anti-CD73 antibody or antigen-binding fragment thereof provided herein comprises a heavy chain comprising an amino acid sequence of SEQ ID NO: 74 and a light chain comprising an amino acid sequence of SEQ ID NO: 78. In some embodiments, an anti-CD73 antibody or antigen-binding fragment thereof provided herein comprises a heavy chain comprising an amino acid sequence of SEQ ID NO: 75 and a light chain comprising an amino acid sequence of SEQ ID NO: 77. In some embodiments, an anti-CD73 antibody or antigen-binding fragment thereof provided herein comprises a heavy chain comprising an amino acid sequence of SEQ ID NO: 75 and a light chain comprising an amino acid sequence of SEQ ID NO: 78. In some embodiments, an anti-CD73 antibody or antigen-binding fragment thereof provided herein comprises a heavy chain comprising an amino acid sequence of SEQ ID NO: 76 and a light chain comprising an amino acid sequence of SEQ ID NO: 77. In some embodiments, an anti-CD73 antibody or antigen-binding fragment thereof provided herein comprises a heavy chain comprising an amino acid sequence of SEQ ID NO: 76 and a light chain comprising an amino acid sequence of SEQ ID NO: 78.

In some embodiments, an anti-CD73 antibody or antigen-binding fragment thereof provided herein comprises a heavy chain comprising an amino acid sequence of SEQ ID NO: 119 and a light chain comprising an amino acid sequence of SEQ ID NO: 77. In some embodiments, an anti-CD73 antibody or antigen-binding fragment thereof provided herein comprises a heavy chain comprising an amino acid sequence of SEQ ID NO: 119 and a light chain comprising an amino acid sequence of SEQ ID NO: 78. In some embodiments, an anti-CD73 antibody or antigen-binding fragment thereof provided herein comprises a heavy chain comprising an amino acid sequence of SEQ ID NO: 120 and a light chain comprising an amino acid sequence of SEQ ID NO: 77. In some embodiments, an anti-CD73 antibody or antigen-binding fragment thereof provided herein comprises a heavy chain comprising an amino acid sequence of SEQ ID NO: 120 and a light chain comprising an amino acid sequence of SEQ ID NO: 78. In some embodiments, an anti-CD73 antibody or antigen-binding fragment thereof provided herein comprises a heavy chain comprising an amino acid sequence of SEQ ID NO: 121 and a light chain comprising an amino acid sequence of SEQ ID NO: 77. In some embodiments, an anti-CD73 antibody or antigen-binding fragment thereof provided herein comprises a heavy chain comprising an amino acid sequence of SEQ ID NO: 121 and a light chain comprising an amino acid sequence of SEQ ID NO: 78. In some embodiments, an anti-CD73 antibody or antigen-binding fragment thereof provided herein comprises a heavy chain comprising an amino acid sequence of SEQ ID NO: 122 and a light chain comprising an amino acid sequence of SEQ ID NO: 77. In some embodiments, an anti-CD73 antibody or antigen-binding fragment thereof provided herein comprises a heavy chain comprising an amino acid sequence of SEQ ID NO: 122 and a light chain comprising an amino acid sequence of SEQ ID NO: 78.

In some embodiments, the anti-CD73 antibody comprises a heavy chain comprising an amino acid sequence of SEQ ID NO: 75 and a light chain comprising an amino acid sequence of SEQ ID NO: 77. In some embodiments, the anti-CD73 antibody comprises a heavy chain comprising an amino acid sequence of SEQ ID NO: 75 and a light chain comprising an amino acid sequence of SEQ ID NO: 78. In some embodiments, the anti-CD73 antibody comprises a heavy chain comprising an amino acid sequence of SEQ ID NO: 76 and a light chain comprising an amino acid sequence of SEQ ID NO: 77. In some embodiments, the anti-CD73 antibody comprises a heavy chain comprising an amino acid sequence of SEQ ID NO: 76 and a light chain comprising an amino acid sequence of SEQ ID NO: 78.

In some embodiments, an anti-CD73 antibody or antigen-binding fragment thereof provided herein comprises a heavy chain amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 73. In some embodiments, an anti-CD73 antibody or antigen-binding fragment thereof provided herein comprises a heavy chain amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 74. In some embodiments, an anti-CD73 antibody or antigen-binding fragment thereof provided herein comprises a heavy chain amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 75. In some embodiments, an anti-CD73 antibody or antigen-binding fragment thereof provided herein comprises a heavy chain amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 76. In some embodiments, an anti-CD73 antibody or antigen-binding fragment thereof provided herein comprises a light chain amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 77. In some embodiments, an anti-CD73 antibody or antigen-binding fragment thereof provided herein comprises a light chain amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 78.

In some embodiments, an anti-CD73 antibody or antigen-binding fragment thereof provided herein comprises a heavy chain amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 119. In some embodiments, an anti-CD73 antibody or antigen-binding fragment thereof provided herein comprises a heavy chain amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 120. In some embodiments, an anti-CD73 antibody or antigen-binding fragment thereof provided herein comprises a heavy chain amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 121. In some embodiments, an anti-CD73 antibody or antigen-binding fragment thereof provided herein comprises a heavy chain amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 122.

In some embodiments, an anti-CD73 antibody or antigen-binding fragment thereof provided herein comprises a heavy chain comprising an amino acid sequence of SEQ ID NO: 79 and a light chain comprising an amino acid sequence of SEQ ID NO: 83. In some embodiments, an anti-CD73 antibody or antigen-binding fragment thereof provided herein comprises a heavy chain comprising an amino acid sequence of SEQ ID NO: 79 and a light chain comprising an amino acid sequence of SEQ ID NO: 84. In some embodiments, an anti-CD73 antibody or antigen-binding fragment thereof provided herein comprises a heavy chain comprising an amino acid sequence of SEQ ID NO: 80 and a light chain comprising an amino acid sequence of SEQ ID NO: 83. In some embodiments, an anti-CD73 antibody or antigen-binding fragment thereof provided herein comprises a heavy chain comprising an amino acid sequence of SEQ ID NO: 80 and a light chain comprising an amino acid sequence of SEQ ID NO: 84. In some embodiments, an anti-CD73 antibody or antigen-binding fragment thereof provided herein comprises a heavy chain comprising an amino acid sequence of SEQ ID NO: 81 and a light chain comprising an amino acid sequence of SEQ ID NO: 83. In some embodiments, an anti-CD73 antibody or antigen-binding fragment thereof provided herein comprises a heavy chain comprising an amino acid sequence of SEQ ID NO: 81 and a light chain comprising an amino acid sequence of SEQ ID NO: 84. In some embodiments, an anti-CD73 antibody or antigen-binding fragment thereof provided herein comprises a heavy chain comprising an amino acid sequence of SEQ ID NO: 82 and a light chain comprising an amino acid sequence of SEQ ID NO: 83. In some embodiments, an anti-CD73 antibody or antigen-binding fragment thereof provided herein comprises a heavy chain comprising an amino acid sequence of SEQ ID NO: 82 and a light chain comprising an amino acid sequence of SEQ ID NO: 84.

In some embodiments, an anti-CD73 antibody or antigen-binding fragment thereof provided herein comprises a heavy chain comprising an amino acid sequence of SEQ ID NO: 123 and a light chain comprising an amino acid sequence of SEQ ID NO: 83. In some embodiments, an anti-CD73 antibody or antigen-binding fragment thereof provided herein comprises a heavy chain comprising an amino acid sequence of SEQ ID NO: 123 and a light chain comprising an amino acid sequence of SEQ ID NO: 84. In some embodiments, an anti-CD73 antibody or antigen-binding fragment thereof provided herein comprises a heavy chain comprising an amino acid sequence of SEQ ID NO: 124 and a light chain comprising an amino acid sequence of SEQ ID NO: 83. In some embodiments, an anti-CD73 antibody or antigen-binding fragment thereof provided herein comprises a heavy chain comprising an amino acid sequence of SEQ ID NO: 124 and a light chain comprising an amino acid sequence of SEQ ID NO: 84. In some embodiments, an anti-CD73 antibody or antigen-binding fragment thereof provided herein comprises a heavy chain comprising an amino acid sequence of SEQ ID NO: 125 and a light chain comprising an amino acid sequence of SEQ ID NO: 83. In some embodiments, an anti-CD73 antibody or antigen-binding fragment thereof provided herein comprises a heavy chain comprising an amino acid sequence of SEQ ID NO: 125 and a light chain comprising an amino acid sequence of SEQ ID NO: 84. In some embodiments, an anti-CD73 antibody or antigen-binding fragment thereof provided herein comprises a heavy chain comprising an amino acid sequence of SEQ ID NO: 126 and a light chain comprising an amino acid sequence of SEQ ID NO: 83. In some embodiments, an anti-CD73 antibody or antigen-binding fragment thereof provided herein comprises a heavy chain comprising an amino acid sequence of SEQ ID NO: 126 and a light chain comprising an amino acid sequence of SEQ ID NO: 84.

In some embodiments, an anti-CD73 antibody or antigen-binding fragment thereof provided herein comprises a heavy chain amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 79. In some embodiments, an anti-CD73 antibody or antigen-binding fragment thereof provided herein comprises a heavy chain amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 80. In some embodiments, an anti-CD73 antibody or antigen-binding fragment thereof provided herein comprises a heavy chain amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 81. In some embodiments, an anti-CD73 antibody or antigen-binding fragment thereof provided herein comprises a heavy chain amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 82. In some embodiments, an anti-CD73 antibody or antigen-binding fragment thereof provided herein comprises a light chain amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 83. In some embodiments, an anti-CD73 antibody or antigen-binding fragment thereof provided herein comprises a light chain amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 84.

In some embodiments, an anti-CD73 antibody or antigen-binding fragment thereof provided herein comprises a heavy chain amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 123. In some embodiments, an anti-CD73 antibody or antigen-binding fragment thereof provided herein comprises a heavy chain amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 124. In some embodiments, an anti-CD73 antibody or antigen-binding fragment thereof provided herein comprises a heavy chain amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 125. In some embodiments, an anti-CD73 antibody or antigen-binding fragment thereof provided herein comprises a heavy chain amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 126.

In some embodiments, an anti-CD73 antibody or antigen-binding fragment thereof provided herein comprises three heavy chain CDRs and three light chain CDRs as follows: HCDR1 comprising SEQ ID NO: 1, HCDR2 comprising SEQ ID NO: 2, HCDR3 comprising SEQ ID NO: 3; LCDR1 comprising SEQ ID NO: 4, LCDR2 comprising SEQ ID NO: 5, and LCDR3 comprising SEQ ID NO: 6, as defined by the Kabat numbering system. In some embodiments, an anti-CD73 antibody or antigen-binding fragment thereof provided herein comprises three heavy chain CDRs and three light chain CDRs as follows: HCDR1 comprising SEQ ID NO: 7, HCDR2 comprising SEQ ID NO: 8, HCDR3 comprising SEQ ID NO: 9; LCDR1 comprising SEQ ID NO: 11, LCDR2 comprising SEQ ID NO: 12, and LCDR3 comprising SEQ ID NO: 13, as defined by the IMGT numbering system. In some embodiments, the anti-CD73 antibody or antigen-binding fragment comprises a heavy chain variable region comprising an amino acid sequence of SEQ ID NO: 49, and a light chain variable region comprising an amino acid sequence of SEQ ID NO: 50. In some embodiments, the anti-CD73 antibody or antigen-binding fragment comprises a heavy chain variable region comprising an amino acid sequence of SEQ ID NO: 49, and a light chain variable region comprising an amino acid sequence of SEQ ID NO: 51. In some embodiments, the anti-CD73 antibody comprises a heavy chain amino acid sequence of SEQ ID NO: 61 and a light chain amino acid sequence of SEQ ID NO: 65; or a heavy chain amino acid sequence of SEQ ID NO: 61 and a light chain amino acid sequence of SEQ ID NO: 66; or a heavy chain amino acid sequence of SEQ ID NO: 62 and a light chain amino acid sequence of SEQ ID NO: 65; or a heavy chain amino acid sequence of SEQ ID NO: 62 and a light chain amino acid sequence of SEQ ID NO: 66; or a heavy chain amino acid sequence of SEQ ID NO: 63 and a light chain amino acid sequence of SEQ ID NO: 65; or a heavy chain amino acid sequence of SEQ ID NO: 63 and a light chain amino acid sequence of SEQ ID NO: 66; or a heavy chain amino acid sequence of SEQ ID NO: 64 and a light chain amino acid sequence of SEQ ID NO: 65; or a heavy chain amino acid sequence of SEQ ID NO: 64 and a light chain amino acid sequence of SEQ ID NO: 66; or a heavy chain amino acid sequence of SEQ ID NO: 111 and a light chain amino acid sequence of SEQ ID NO: 65; or a heavy chain amino acid sequence of SEQ ID NO: 111 and a light chain amino acid sequence of SEQ ID NO: 66; or a heavy chain amino acid sequence of SEQ ID NO: 112 and a light chain amino acid sequence of SEQ ID NO: 65; or a heavy chain amino acid sequence of SEQ ID NO: 112 and a light chain amino acid sequence of SEQ ID NO: 66; or a heavy chain amino acid sequence of SEQ ID NO: 113 and a light chain amino acid sequence of SEQ ID NO: 65; or a heavy chain amino acid sequence of SEQ ID NO: 113 and a light chain amino acid sequence of SEQ ID NO: 66; or a heavy chain amino acid sequence of SEQ ID NO: 114 and a light chain amino acid sequence of SEQ ID NO: 65; or a heavy chain amino acid sequence of SEQ ID NO: 114 and a light chain amino acid sequence of SEQ ID NO: 66. In some embodiments, the anti-CD73 antibody comprises a heavy chain amino acid sequence of SEQ ID NO: 63 and a light chain amino acid sequence of SEQ ID NO: 65. In some embodiments, the anti-CD73 antibody comprises a heavy chain amino acid sequence of SEQ ID NO: 63 and a light chain amino acid sequence of SEQ ID NO: 66. In some embodiments, the anti-CD73 antibody comprises a heavy chain amino acid sequence of SEQ ID NO: 64 and a light chain amino acid sequence of SEQ ID NO: 65. In some embodiments, the anti-CD73 antibody comprises a heavy chain amino acid sequence of SEQ ID NO: 64 and a light chain amino acid sequence of SEQ ID NO: 66. In some embodiments, the anti-CD73 antibody comprises a heavy chain amino acid sequence of SEQ ID NO: 113 and a light chain amino acid sequence of SEQ ID NO: 65. In some embodiments, the anti-CD73 antibody comprises a heavy chain amino acid sequence of SEQ ID NO: 113 and a light chain amino acid sequence of SEQ ID NO: 66. In some embodiments, the anti-CD73 antibody comprises a heavy chain amino acid sequence of SEQ ID NO: 114 and a light chain amino acid sequence of SEQ ID NO: 65. In some embodiments, the anti-CD73 antibody comprises a heavy chain amino acid sequence of SEQ ID NO: 114 and a light chain amino acid sequence of SEQ ID NO: 66.

In some embodiments, an anti-CD73 antibody or antigen-binding fragment thereof provided herein comprises three heavy chain CDRs and three light chain CDRs as follows: HCDR1 comprising SEQ ID NO: 14, HCDR2 comprising SEQ ID NO: 15, HCDR3 comprising SEQ ID NO: 16; LCDR1 comprising SEQ ID NO: 17, LCDR2 comprising SEQ ID NO: 18, and LCDR3 comprising SEQ ID NO: 19, as defined by the Kabat numbering system. In some embodiments, an anti-CD73 antibody or antigen-binding fragment thereof provided herein comprises three heavy chain CDRs and three light chain CDRs as follows: HCDR1 comprising SEQ ID NO: 20, HCDR2 comprising SEQ ID NO: 21, HCDR3 comprising SEQ ID NO: 22; LCDR1 comprising SEQ ID NO: 23, LCDR2 comprising SEQ ID NO: 24, and LCDR3 comprising SEQ ID NO: 25, as defined by the IMGT numbering system. In some embodiments, the anti-CD73 antibody or antigen-binding fragment comprises a heavy chain variable region comprising an amino acid sequence of SEQ ID NO: 52, and a light chain variable region comprising an amino acid sequence of SEQ ID NO: 53. In some embodiments, the anti-CD73 antibody or antigen-binding fragment comprises a heavy chain variable region comprising an amino acid sequence of SEQ ID NO: 52, and a light chain variable region comprising an amino acid sequence of SEQ ID NO: 54. In some embodiments, the anti-CD73 antibody comprises a heavy chain amino acid sequence of SEQ ID NO: 67 and a light chain amino acid sequence of SEQ ID NO: 71; or a heavy chain amino acid sequence of SEQ ID NO: 67 and a light chain amino acid sequence of SEQ ID NO: 72; or a heavy chain amino acid sequence of SEQ ID NO: 68 and a light chain amino acid sequence of SEQ ID NO: 71; or a heavy chain amino acid sequence of SEQ ID NO: 68 and a light chain amino acid sequence of SEQ ID NO: 72; or a heavy chain amino acid sequence of SEQ ID NO: 69 and a light chain amino acid sequence of SEQ ID NO: 71; or a heavy chain amino acid sequence of SEQ ID NO: 69 and a light chain amino acid sequence of SEQ ID NO: 72; or a heavy chain amino acid sequence of SEQ ID NO: 70 and a light chain amino acid sequence of SEQ ID NO: 71; or a heavy chain amino acid sequence of SEQ ID NO: 70 and a light chain amino acid sequence of SEQ ID NO: 72; or a heavy chain amino acid sequence of SEQ ID NO: 115 and a light chain amino acid sequence of SEQ ID NO: 71; or a heavy chain amino acid sequence of SEQ ID NO: 115 and a light chain amino acid sequence of SEQ ID NO: 72; or a heavy chain amino acid sequence of SEQ ID NO: 116 and a light chain amino acid sequence of SEQ ID NO: 71; or a heavy chain amino acid sequence of SEQ ID NO: 116 and a light chain amino acid sequence of SEQ ID NO: 72; or a heavy chain amino acid sequence of SEQ ID NO: 117 and a light chain amino acid sequence of SEQ ID NO: 71; or a heavy chain amino acid sequence of SEQ ID NO: 117 and a light chain amino acid sequence of SEQ ID NO: 72; or a heavy chain amino acid sequence of SEQ ID NO: 118 and a light chain amino acid sequence of SEQ ID NO: 71; or a heavy chain amino acid sequence of SEQ ID NO: 118 and a light chain amino acid sequence of SEQ ID NO: 72. In some embodiments, the anti-CD73 antibody comprises a heavy chain amino acid sequence of SEQ ID NO: 69 and a light chain amino acid sequence of SEQ ID NO: 71. In some embodiments, the anti-CD73 antibody comprises a heavy chain amino acid sequence of SEQ ID NO: 69 and a light chain amino acid sequence of SEQ ID NO: 72. In some embodiments, the anti-CD73 antibody comprises a heavy chain amino acid sequence of SEQ ID NO: 70 and a light chain amino acid sequence of SEQ ID NO: 71. In some embodiments, the anti-CD73 antibody comprises a heavy chain amino acid sequence of SEQ ID NO: 70 and a light chain amino acid sequence of SEQ ID NO: 72. In some embodiments, the anti-CD73 antibody comprises a heavy chain amino acid sequence of SEQ ID NO: 117 and a light chain amino acid sequence of SEQ ID NO: 71. In some embodiments, the anti-CD73 antibody comprises a heavy chain amino acid sequence of SEQ ID NO: 117 and a light chain amino acid sequence of SEQ ID NO: 72. In some embodiments, the anti-CD73 antibody comprises a heavy chain amino acid sequence of SEQ ID NO: 118 and a light chain amino acid sequence of SEQ ID NO: 71. In some embodiments, the anti-CD73 antibody comprises a heavy chain amino acid sequence of SEQ ID NO: 118 and a light chain amino acid sequence of SEQ ID NO: 72.

In some embodiments, an anti-CD73 antibody or antigen-binding fragment thereof provided herein comprises three heavy chain CDRs and three light chain CDRs as follows: HCDR1 comprising SEQ ID NO: 26, HCDR2 comprising SEQ ID NO: 27, HCDR3 comprising SEQ ID NO: 28; LCDR1 comprising SEQ ID NO: 29, LCDR2 comprising SEQ ID NO: 30, and LCDR3 comprising SEQ ID NO: 31, as defined by the Kabat numbering system. In some embodiments, an anti-CD73 antibody or antigen-binding fragment thereof provided herein comprises three heavy chain CDRs and three light chain CDRs as follows: HCDR1 comprising SEQ ID NO: 32, HCDR2 comprising SEQ ID NO: 33, HCDR3 comprising SEQ ID NO: 34; LCDR1 comprising SEQ ID NO: 35, LCDR2 comprising SEQ ID NO: 36, and LCDR3 comprising SEQ ID NO: 37, as defined by the IMGT numbering system. In some embodiments, the anti-CD73 antibody or antigen-binding fragment comprises a heavy chain variable region comprising an amino acid sequence of SEQ ID NO: 55, and a light chain variable region comprising an amino acid sequence of SEQ ID NO: 56. In some embodiments, the anti-CD73 antibody or antigen-binding fragment comprises a heavy chain variable region comprising an amino acid sequence of SEQ ID NO: 55, and a light chain variable region comprising an amino acid sequence of SEQ ID NO: 57. In some embodiments, the anti-CD73 antibody comprises a heavy chain amino acid sequence of SEQ ID NO: 73 and a light chain amino acid sequence of SEQ ID NO: 77; or a heavy chain amino acid sequence of SEQ ID NO: 73 and a light chain amino acid sequence of SEQ ID NO: 78; or a heavy chain amino acid sequence of SEQ ID NO: 74 and a light chain amino acid sequence of SEQ ID NO: 77; or a heavy chain amino acid sequence of SEQ ID NO: 74 and a light chain amino acid sequence of SEQ ID NO: 78; or a heavy chain amino acid sequence of SEQ ID NO: 75 and a light chain amino acid sequence of SEQ ID NO: 77; or a heavy chain amino acid sequence of SEQ ID NO: 75 and a light chain amino acid sequence of SEQ ID NO: 78; or a heavy chain amino acid sequence of SEQ ID NO: 76 and a light chain amino acid sequence of SEQ ID NO: 77; or a heavy chain amino acid sequence of SEQ ID NO: 76 and a light chain amino acid sequence of SEQ ID NO: 78; or a heavy chain amino acid sequence of SEQ ID NO: 119 and a light chain amino acid sequence of SEQ ID NO: 77; or a heavy chain amino acid sequence of SEQ ID NO: 119 and a light chain amino acid sequence of SEQ ID NO: 78; or a heavy chain amino acid sequence of SEQ ID NO: 120 and a light chain amino acid sequence of SEQ ID NO: 77; or a heavy chain amino acid sequence of SEQ ID NO: 120 and a light chain amino acid sequence of SEQ ID NO: 78; or a heavy chain amino acid sequence of SEQ ID NO: 121 and a light chain amino acid sequence of SEQ ID NO: 77; or a heavy chain amino acid sequence of SEQ ID NO: 121 and a light chain amino acid sequence of SEQ ID NO: 78; or a heavy chain amino acid sequence of SEQ ID NO: 122 and a light chain amino acid sequence of SEQ ID NO: 77; or a heavy chain amino acid sequence of SEQ ID NO: 122 and a light chain amino acid sequence of SEQ ID NO: 78. In some embodiments, the anti-CD73 antibody comprises a heavy chain amino acid sequence of SEQ ID NO: 75 and a light chain amino acid sequence of SEQ ID NO: 77. In some embodiments, the anti-CD73 antibody comprises a heavy chain amino acid sequence of SEQ ID NO: 75 and a light chain amino acid sequence of SEQ ID NO: 78. In some embodiments, the anti-CD73 antibody comprises a heavy chain amino acid sequence of SEQ ID NO: 76 and a light chain amino acid sequence of SEQ ID NO: 77. In some embodiments, the anti-CD73 antibody comprises a heavy chain amino acid sequence of SEQ ID NO: 76 and a light chain amino acid sequence of SEQ ID NO: 78. In some embodiments, the anti-CD73 antibody comprises a heavy chain amino acid sequence of SEQ ID NO: 121 and a light chain amino acid sequence of SEQ ID NO: 77. In some embodiments, the anti-CD73 antibody comprises a heavy chain amino acid sequence of SEQ ID NO: 121 and a light chain amino acid sequence of SEQ ID NO: 78. In some embodiments, the anti-CD73 antibody comprises a heavy chain amino acid sequence of SEQ ID NO: 122 and a light chain amino acid sequence of SEQ ID NO: 77. In some embodiments, the anti-CD73 antibody comprises a heavy chain amino acid sequence of SEQ ID NO: 122 and a light chain amino acid sequence of SEQ ID NO: 78.

In some embodiments, an anti-CD73 antibody or antigen-binding fragment thereof provided herein comprises three heavy chain CDRs and three light chain CDRs as follows: HCDR1 comprising SEQ ID NO: 38, HCDR2 comprising SEQ ID NO: 39, HCDR3 comprising SEQ ID NO: 40; LCDR1 comprising SEQ ID NO: 41, LCDR2 comprising SEQ ID NO: 42, and LCDR3 comprising SEQ ID NO: 43, as defined by the Kabat numbering system. In some embodiments, an anti-CD73 antibody or antigen-binding fragment thereof provided herein comprises three heavy chain CDRs and three light chain CDRs as follows: HCDR1 comprising SEQ ID NO: 44, HCDR2 comprising SEQ ID NO: 45, HCDR3 comprising SEQ ID NO: 46; LCDR1 comprising SEQ ID NO: 47, LCDR2 comprising SEQ ID NO: 48, and LCDR3 comprising SEQ ID NO: 49, as defined by the IMGT numbering system. In some embodiments, the anti-CD73 antibody or antigen-binding fragment comprises a heavy chain variable region comprising an amino acid sequence of SEQ ID NO: 58, and a light chain variable region comprising an amino acid sequence of SEQ ID NO: 59. In some embodiments, the anti-CD73 antibody or antigen-binding fragment comprises a heavy chain variable region comprising an amino acid sequence of SEQ ID NO: 58, and a light chain variable region comprising an amino acid sequence of SEQ ID NO: 60. In some embodiments, the anti-CD73 antibody comprises a heavy chain amino acid sequence of SEQ ID NO: 79 and a light chain amino acid sequence of SEQ ID NO: 83; or a heavy chain amino acid sequence of SEQ ID NO: 79 and a light chain amino acid sequence of SEQ ID NO: 84; or a heavy chain amino acid sequence of SEQ ID NO: 80 and a light chain amino acid sequence of SEQ ID NO: 83; or a heavy chain amino acid sequence of SEQ ID NO: 80 and a light chain amino acid sequence of SEQ ID NO: 84; or a heavy chain amino acid sequence of SEQ ID NO: 81 and a light chain amino acid sequence of SEQ ID NO: 83; or a heavy chain amino acid sequence of SEQ ID NO: 81 and a light chain amino acid sequence of SEQ ID NO: 84; or a heavy chain amino acid sequence of SEQ ID NO: 82 and a light chain amino acid sequence of SEQ ID NO: 83; or a heavy chain amino acid sequence of SEQ ID NO: 82 and a light chain amino acid sequence of SEQ ID NO: 84; or a heavy chain amino acid sequence of SEQ ID NO: 123 and a light chain amino acid sequence of SEQ ID NO: 83; or a heavy chain amino acid sequence of SEQ ID NO: 123 and a light chain amino acid sequence of SEQ ID NO: 84; or a heavy chain amino acid sequence of SEQ ID NO: 124 and a light chain amino acid sequence of SEQ ID NO: 83; or a heavy chain amino acid sequence of SEQ ID NO: 124 and a light chain amino acid sequence of SEQ ID NO: 84; or a heavy chain amino acid sequence of SEQ ID NO: 125 and a light chain amino acid sequence of SEQ ID NO: 83; or a heavy chain amino acid sequence of SEQ ID NO: 125 and a light chain amino acid sequence of SEQ ID NO: 84; or a heavy chain amino acid sequence of SEQ ID NO: 126 and a light chain amino acid sequence of SEQ ID NO: 83; or a heavy chain amino acid sequence of SEQ ID NO: 126 and a light chain amino acid sequence of SEQ ID NO: 84. In some embodiments, the anti-CD73 antibody comprises a heavy chain amino acid sequence of SEQ ID NO: 81 and a light chain amino acid sequence of SEQ ID NO: 83. In some embodiments, the anti-CD73 antibody comprises a heavy chain amino acid sequence of SEQ ID NO: 81 and a light chain amino acid sequence of SEQ ID NO: 84. In some embodiments, the anti-CD73 antibody comprises a heavy chain amino acid sequence of SEQ ID NO: 82 and a light chain amino acid sequence of SEQ ID NO: 83. In some embodiments, the anti-CD73 antibody comprises a heavy chain amino acid sequence of SEQ ID NO: 82 and a light chain amino acid sequence of SEQ ID NO: 84. In some embodiments, the anti-CD73 antibody comprises a heavy chain amino acid sequence of SEQ ID NO: 125 and a light chain amino acid sequence of SEQ ID NO: 83. In some embodiments, the anti-CD73 antibody comprises a heavy chain amino acid sequence of SEQ ID NO: 125 and a light chain amino acid sequence of SEQ ID NO: 84. In some embodiments, the anti-CD73 antibody comprises a heavy chain amino acid sequence of SEQ ID NO: 126 and a light chain amino acid sequence of SEQ ID NO: 83. In some embodiments, the anti-CD73 antibody comprises a heavy chain amino acid sequence of SEQ ID NO: 126 and a light chain amino acid sequence of SEQ ID NO: 84.

In some embodiments, the disclosed anti-CD73 antibodies demonstrate an increased safety profile due to substitution of residues in the Fc domain. In some embodiments, the disclosed anti-CD73 antibodies comprise an IgG1 Fc domain that has been mutated to reduce binding to a Fcγ receptor (FcγR) as compared to an IgG1 Fc-containing antibody with a wild type IgG1 Fc domain. These substitutions reduce the ability of the antibody to bind to various cell receptors, such as a Fcγ receptor (FcγR), and other immune molecules, as compared to an anti-CD73 antibody without these substitutions. Without being bound by theory, the reduced binding of an antibody to a FcγR may reduce non-antigen mediated uptake by neutrophils, thereby reducing neutropenia in a treated subject.

In some embodiments, the disclosed anti-CD73 antibodies demonstrate superior properties (e.g., improved stability (e.g., shelf and/or serum stability), antigen-binding specificity (e.g., epitope and/or affinity), safety profile, in vivo anti-tumor activity) compared to other CD73 antibodies. In some embodiments, the disclosed anti-CD73 antibodies demonstrate superior antigen-binding specificity compared to other CD73 antibodies. In some embodiments, the disclosed anti-CD73 antibodies demonstrate superior site-specific linker-payload conjugation compared to other CD73 antibodies. In some embodiments, the disclosed anti-CD73 antibodies demonstrate a superior safety profile in vivo compared to other CD73 antibodies.

In any of the antibodies or antigen-binding fragments disclosed herein, the heavy chain amino acid sequence may lack the C-terminal lysine. In some embodiments, the anti-CD73 antibody or antigen-binding fragment comprises a heavy chain sequence selected from SEQ ID NOs: 111-126.

In various embodiments, amino acid substitutions may be made while retaining the binding affinity and/or specificity of an antibody disclosed herein and/or to provide one or more additional beneficial property, e.g., by making one or more changes in framework, constant domain, and/or CDR sequences. In some embodiments, the substitutions are of single residues. For instance, in some embodiments, the anti-CD73 antibodies comprise a human IgG1 Fc domain that comprises amino acid substitutions to reduce binding to an FcγR as compared to an IgG1 Fc-containing antibody with a wild type IgG1 Fc domain. In some embodiments, the anti-CD73 antibodies comprise a mutated human IgG1 Fc domain that comprises one or more substitution selected from N297Q, N297A, L234G/L235G, L234A/L235A, L234A/L235A/D265S, L234A/L235A/P329G, L234A/L235A/P238S/H268Q/K274Q, L235G/G236R, G236R/L328R, and L234S/L235T/G236R. In some embodiments, the anti-CD73 antibodies comprise a mutated human IgG1 Fc domain that comprises the substitutions L234A, L235A, P238S, H268Q, and K274Q. Insertions usually will be on the order of from about 1 to about 20 amino acid residues, although considerably larger insertions may be tolerated as long as biological function is retained (e.g., binding to CD73). Deletions usually range from about 1 to about 20 amino acid residues, although in some cases deletions may be much larger. Substitutions, deletions, insertions, or any combination thereof may be used to arrive at a final derivative or variant. Generally, these changes are done on a few amino acids to minimize the alteration of the molecule, particularly the immunogenicity and specificity of the antigen binding protein. However, larger changes may be tolerated in certain circumstances. Conservative substitutions are generally made in accordance with the following chart depicted in Table 10.

TABLE 10
Conservative amino acid substitutions

| Original Residue | Exemplary Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Substantial changes in function or immunological identity are made by selecting substitutions that are less conservative than those shown in Table 10. For example, substitutions may be made which more significantly affect: the structure of the polypeptide backbone in the area of the alteration, for example the alpha-helical or beta-sheet structure; the charge or hydrophobicity of the molecule at the target site; or the bulk of the side chain. The substitutions which in general are expected to produce the greatest changes in the polypeptide's properties are those in which (a) a hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine.

In various embodiments where variant antibody sequences are used in an ADC, the variants typically exhibit the same qualitative biological activity and will elicit the same immune response, although variants may also be selected to modify the characteristics of the antigen-binding proteins as needed. For example, the anti-CD73 antibodies provided herein may comprise a human IgG1 Fc domain that is mutated to reduce binding to an FcγR as compared to an IgG1 Fc-containing antibody with a wild type IgG1 Fc domain. Alternatively, the variant may be designed such that the biological activity of the antigen binding protein is altered. For example, glycosylation sites may be altered or removed.

Any of the anti-CD73 antibodies and antigen binding fragments disclosed herein may be used as a conjugate, e.g., with a detectable agent and/or another therapeutic agent. In some embodiments the anti-CD73 antibody or antigen-binding fragment may be used in an antibody-drug conjugate (ADC), e.g., any of the ADCs disclosed herein, preferably to target the drug in the ADC to a cancer cell. As shown below, the linker-toxins in the ADCs disclosed herein are surprisingly effective with the anti-CD73 antibodies also disclosed herein. These antibodies may be used with the linkers and toxin (e.g., Compound 1) disclosed herein.

### Linkers

In various embodiments, the anti-CD73 antibodies and antigen-binding fragments disclosed herein may be joined to a drug moiety (e.g., a cytotoxic payload, e.g., Compound 1) by a linker to create an antibody-drug conjugate (ADC).

In some embodiments, the linker in an ADC is stable extracellularly in a sufficient manner to be therapeutically effective. In some embodiments, the linker is stable outside a cell, such that the ADC remains intact when present in extracellular conditions (e.g., prior to transport or delivery into a cell). The term “intact,” used in the context of an ADC, means that the antibody moiety remains attached to the drug moiety (e.g., Compound 1). As used herein, “stable,” in the context of a linker or ADC comprising a linker, means that no more than about 20%, no more than about 15%, no more than about 10%, no more than about 5%, no more than about 3%, or no more than about 1% of the linkers (or any percentage in between) in a sample of ADC are cleaved (or in the case of an overall ADC are otherwise not intact) when the ADC is present in extracellular conditions when evaluated over a set period of time. In some embodiments, the linkers in ADCs disclosed herein are chosen to remain stable for more than about 48 hours, more than about 60 hours, more than about 72 hours, more than about 84 hours, or more than about 96 hours.

Whether a linker is stable extracellularly can be determined, for example, by including an ADC in plasma for a predetermined time period (e.g., 2, 4, 6, 8, 16, or 24 hours) and then quantifying the amount of free drug moiety present in the plasma. Stability may allow the ADC time to localize to target tumor cells and prevent the premature release of the drug, which could lower the therapeutic index of the ADC by indiscriminately damaging both normal and tumor tissues. In some embodiments, the linker is stable outside of a target cell and releases the drug moiety from the ADC once inside of the cell, such that the drug moiety can bind to its target (e.g., to STING). Thus, an effective linker will: (i) maintain the specific binding properties of the antibody moiety; (ii) allow delivery, e.g., intracellular delivery, of the drug moiety via stable attachment to the antibody moiety; (iii) remain stable and intact until the ADC has been transported or delivered to its target site; and (iv) allow for the therapeutic effect, e.g., cytotoxic effect, of the drug moiety after cleavage.

Linkers may impact the physico-chemical properties of an ADC. As many cytotoxic agents are hydrophobic in nature, linking them to the antibody with an additional hydrophobic moiety may lead to aggregation. ADC aggregates are insoluble and often limit achievable drug loading onto the antibody, which can negatively affect the potency of the ADC. Protein aggregates of biologics, in general, have also been linked to increased immunogenicity. As shown below, linkers disclosed herein result in ADCs with low aggregation levels and desirable levels of drug loading. In various embodiments, a linker is conjugated to the antibody or antigen-binding fragment through a cysteine. In various embodiments, a linker is conjugated to the antibody or antigen-binding fragment through a lysine. Suitable methods for conjugating linkers of the present disclosure to an antibody include the technologies for directed attachment to a lysine on a heavy chain of an antibody, to a cysteine on the heavy chain of an antibody, and to a cysteine on the light chain of an antibody, e.g., as disclosed in PCT applications WO 2017/213267, WO 2017/106643, and WO 2016/205618, and in Junutula et al. (2008) Journal of Immunological Methods 332:41-52, all of which are herein incorporated by reference in their entireties. In some embodiments, a linker is conjugated to the antibody or antigen-binding fragment on the light chain, e.g., at a cysteine on the light chain, e.g., at cysteine-80 on the light chain. In some embodiments, a linker is conjugated to the antibody or antigen-binding fragment on the heavy chain, e.g., at a cysteine on the heavy chain, e.g., at cysteine-118 on the heavy chain.

A linker used herein may be “cleavable” or “non-cleavable.” See, e.g., Ducry and Stump, Bioconjugate Chem. (2010) 21:5-13. Cleavable linkers are designed to release the drug when subjected to certain environment factors, e.g., when internalized into the target cell, whereas non-cleavable linkers generally rely on the degradation of the antibody moiety itself.

In some embodiments, the linker is a non-cleavable linker. In some embodiments, the drug moiety of the ADC is released by degradation of the antibody moiety.

In some embodiments, the linker is cleavable. Cleavable linkers are designed to release the drug when subjected to certain environmental factors, e.g., when internalized into the target cell. A cleavable linker refers to any linker that comprises a cleavable moiety. As used herein, the term “cleavable moiety” refers to any chemical bond that can be cleaved. Suitable cleavable chemical bonds are known in the art and include, but are not limited to, acid labile bonds, protease/peptidase labile bonds, photolabile bonds, disulfide bonds, and esterase labile bonds. Linkers comprising a cleavable moiety can allow for the release of the drug moiety from the ADC via cleavage at a particular site in the linker.

In some embodiments, the linker is cleavable under intracellular conditions, such that cleavage of the linker sufficiently releases the drug moiety from the antibody moiety in the intracellular environment to activate the drug and/or render the drug therapeutically effective. In some embodiments, the drug moiety is not cleaved from the antibody moiety until the ADC enters a cell that expresses an antigen specific for the antibody moiety of the ADC, and the drug moiety is cleaved from the antibody moiety upon entering the cell. In some embodiments, the linker comprises a cleavable moiety that is positioned such that no part of the linker or the antibody moiety remains bound to the drug moiety upon cleavage. Exemplary cleavable linkers include acid labile linkers, protease/peptidase-sensitive linkers, photolabile linkers, dimethyl-, disulfide-, or sulfonamide-containing linkers.

In some embodiments, the linker is cleavable by a cleaving agent, e.g., an enzyme, that is present in the intracellular environment (e.g., within a lysosome, endosome, or caveolea). The linker can be, e.g., a peptide linker that is cleaved by an intracellular peptidase or protease enzyme, including, but not limited to, a lysosomal or endosomal protease. In some embodiments, the linker is a cleavable peptide linker. As used herein, a cleavable peptide linker refers to any linker that comprises a cleavable peptide moiety. The term “cleavable peptide moiety” refers to any chemical bond linking amino acids (natural or synthetic amino acid derivatives) that can be cleaved by an agent that is present in the intracellular environment. In some embodiments, a cleavable peptide linker is more stably conjugated to an antibody disclosed herein compared to an acid labile linker.

In some embodiments, the linker is an enzyme-cleavable linker and a cleavable peptide moiety in the linker is cleavable by the enzyme. In some embodiments, the cleavable peptide moiety is cleavable by a lysosomal enzyme, e.g., cathepsin or legumain (also known as asparaginyl endopeptidase or vacuolar processing enzyme). In some embodiments, the linker is a cathepsin-cleavable linker. In some embodiments, the linker is a legumain-cleavable linker. In some embodiments, the cleavable peptide moiety in the linker is cleavable by a lysosomal cysteine cathepsin, such as cathepsin B, C, F, H, K, L, O, S, V, X, or W. In some embodiments, the cleavable peptide moiety is cleavable by cathepsin B. An exemplary dipeptide that may be cleaved by cathepsin B is valine-citrulline (Val-Cit). See, e.g., Dubowchik et al. (2002) Bioconjugate Chem. 13:855-69. Another exemplary dipeptide that may be cleaved by cathepsin B is valine-alanine (Val-Ala). See, e.g., Fu and Ho (2002) Antib. Ther. 1(2):33-43.

In some embodiments, the cleavable peptide moiety in the linker is cleavable by a lysosomal cysteine endopeptidase, such as legumain. An exemplary monopeptide that may be cleaved by legumain is asparagine (Asn). Another exemplary monopeptide that may be cleaved by legumain is aspartic acid (Asp).

In some embodiments, the linker or the cleavable peptide moiety in the linker comprises an amino acid unit. In some embodiments, the amino acid unit allows for cleavage of the linker by a protease, thereby facilitating release of the drug moiety from the ADC upon exposure to one or more intracellular proteases, such as one or more lysosomal enzymes. See, e.g., Doronina et al. (2003) Nat. Biotechnol. 21:778-84; and Dubowchik and Walker (1999) Pharm. Therapeutics 83:67-123. Exemplary amino acid units include, but are not limited to, monopeptides, dipeptides, tripeptides, tetrapeptides, and pentapeptides. Exemplary monopeptides include, but are not limited to, asparagine (Asn) and aspartic acid (Asp). Exemplary dipeptides include, but are not limited to, valine-citrulline (Val-Cit), alanine-asparagine (Ala-Asn), alanine-phenylalanine (Ala-Phe), phenylalanine-lysine (Phe-Lys), alanine-lysine (Ala-Lys), alanine-valine (Ala-Val), valine-alanine (Val-Ala), valine-lysine (Val-Lys), lysine-lysine (Lys-Lys), phenylalanine-citrulline (Phe-Cit), leucine-citrulline (Leu-Cit), isoleucine-citrulline (Ile-Cit), tryptophan-citrulline (Trp-Cit), and phenylalanine-alanine (Phe-Ala). Exemplary tripeptides include, but are not limited to, alanine-alanine-asparagine (Ala-Ala-Asn), glycine-valine-citrulline (Gly-Val-Cit), glutamic acid-valine-citrulline, glycine-glycine-glycine (Gly-Gly-Gly), phenylalanine-phenylalanine-lysine (Phe-Phe-Lys), alanine-phenylalanine-lysine (Ala-Phe-Lys), glycine-valine-alanine (Gly-Val-Ala), and glycine-phenylalanine-lysine (Gly-Phe-Lys). Exemplary tetrapeptides include, but are not limited to, Gly-Gly-Phe-Gly (SEQ ID NO: 244). Other exemplary amino acid units include, but are not limited to, Gly-Phe-Leu-Gly (SEQ ID NO: 245), Ala-Leu-Ala-Leu (SEQ ID NO: 246), Phe-N9-tosyl-Arg, and Phe-N9-Nitro-Arg, as described in, e.g., U.S. Pat. No. 6,214,345. In some embodiments, an amino acid unit may comprise amino acid residues comprising at least one methyl group, e.g., a monomethyl or dimethyl group. Exemplary amino acid units that comprise amino acid residues comprising at least one methyl group include, but are not limited to, N-methylated alanine ((NMe)Ala), methylated aspartic acid (Asp(OMe)), valine dimethylated lysine (Val-Lys(Me)₂), and alanine dimethylated lysine (Ala-Lys(Me)₂). In some embodiments, the amino acid unit in the linker comprises Val-Ala. In some embodiments, the amino acid unit in the linker comprises Val-Cit. An amino acid unit may comprise amino acid residues that occur naturally and/or minor amino acids and/or non-naturally occurring amino acid analogs, such as citrulline. Amino acid units can be designed and optimized for enzymatic cleavage by a particular enzyme, for example, a tumor-associated protease, a lysosomal protease such as legumain or cathepsin B, C, D, or S.

In some embodiments, the linker in an ADC disclosed herein may comprise an antibody attachment moiety. An antibody attachment moiety may be used, for example, to link the antibody moiety to the linker, which in turn may link to the drug moiety, e.g., indirectly through a cleavable moiety (e.g., a cleavable peptide).

In some embodiments, the linker comprises an antibody attachment moiety comprising a maleimide moiety (Mal). The term “maleimide moiety,” as used herein, means a compound that contains a maleimide group and that is reactive with a sulfhydryl group, e.g., a sulfhydryl group of a cysteine residue on the antibody moiety. Other functional groups that are reactive with sulfhydryl groups (thiols) and may therefore be used in place of a Mal include, but are not limited to, iodoacetamide, bromoacetamide, vinyl pyridine, disulfide, pyridyl disulfide, isocyanate, and isothiocyanate.

In some embodiments, the linker attaches to the antibody or antigen-binding fragment via a Mal moiety. In some embodiments, the Mal moiety is reactive with a cysteine residue on the antibody or antigen-binding fragment. In some embodiments, the Mal moiety is joined to the antibody or antigen-binding fragment via the cysteine residue.

In some embodiments, the Mal moiety is a MC moiety. In some embodiments, the linker attaches to the antibody or antigen-binding fragment via an MC moiety. In some embodiments, the MC moiety is reactive with a cysteine residue on the antibody or antigen-binding fragment. In some embodiments, the MC moiety is joined to the antibody or antigen-binding fragment via the cysteine residue.

In some embodiments, the linker comprises a Mal moiety and a cleavable peptide moiety. In some embodiments, the cleavable peptide moiety comprises an amino acid unit. In some embodiments, the amino acid unit comprises Val-Cit. In some embodiments, the amino acid unit comprises Val-Ala. In some embodiments, the amino acid unit comprises Gly-Gly-Phe-Gly (SEQ ID NO: 244). In some embodiments, the Mal moiety attaches the antibody moiety to the cleavable peptide moiety in the linker. In some embodiments, the cleavable peptide moiety comprises an amino acid unit. In some embodiments, the amino acid unit comprises Val-Cit. In some embodiments, the amino acid unit comprises Val-Ala. In some embodiments, the amino acid unit comprises Gly-Gly-Phe-Gly (SEQ ID NO: 244). In some embodiments, the linker comprises Mal-Val-Cit. In some embodiments, the linker comprises Mal-Val-Ala. In some embodiments, the linker comprises Mal-Gly-Gly-Phe-Gly (SEQ ID NO: 252).

In some embodiments, the linker comprises an MC moiety and a cleavable peptide moiety. In some embodiments, the cleavable peptide moiety comprises an amino acid unit. In some embodiments, the amino acid unit comprises Val-Cit. In some embodiments, the amino acid unit comprises Val-Ala. In some embodiments, the amino acid unit comprises Gly-Gly-Phe-Gly (SEQ ID NO: 244). In some embodiments, the MC moiety attaches the antibody moiety to the cleavable peptide moiety in the linker. In some embodiments, the cleavable peptide moiety comprises an amino acid unit. In some embodiments, the amino acid unit comprises Val-Cit. In some embodiments, the amino acid unit comprises Val-Ala. In some embodiments, the amino acid unit comprises Gly-Gly-Phe-Gly (SEQ ID NO: 244). In some embodiments, the linker comprises MC-Val-Cit. In some embodiments, the linker comprises MC-Val-Ala. In some embodiments, the linker comprises MC-Gly-Gly-Phe-Gly (SEQ ID NO: 249).

In some embodiments, any of the linkers in ADCs disclosed herein may comprise at least one spacer unit joining the antibody moiety to the drug moiety. In some embodiments, the spacer unit joins a cleavage site (e.g., a cleavable peptide moiety) in the linker to the antibody moiety. In some embodiments, the spacer unit joins a cleavage site (e.g., a cleavable peptide moiety) in the linker to the drug moiety. In some embodiments, the linker, and/or spacer unit in the linker, is substantially hydrophilic. In some embodiments, the linker includes one or more polyethylene glycol (PEG) moieties, e.g., 1, 2, 3, or 4 PEG moieties. In some embodiments, the linker includes one or more alkyl moieties, e.g., 1, 2, 3, 4, or 5 alkyl moieties.

In some embodiments, the spacer unit in the linker comprises one or more PEG moieties. In some embodiments, the spacer unit comprises -(PEG)ₘ-, and m is an integer from 1 to 10. In some embodiments, m ranges from 1 to 4; or from 2 to 4. In some embodiments, m is 2. In some embodiments, m is 3. In some embodiments, m is 4. In some embodiments, the spacer unit comprises (PEG)₂, (PEG)₃, or (PEG)₄. In some embodiments, the spacer unit comprises PEG₂-Lys(ε-PEG₈-OMe)-PEG₂.

In some embodiments, the spacer unit in the linker comprises an alkyl moiety. In some embodiments, the spacer unit comprises —(CH₂)ₙ—, and n is an integer from 1 to 10 (i.e., n may be 1, 2, 3, 4, 5, 6, 7, 8, 9, or 10). In some embodiments, n is 3, 4, or 5. In some embodiments, the spacer unit comprises (CH₂)₃, or (CH₂)₄, or (CH₂)₅. In some embodiments, the spacer unit comprises CH₂—CH₂.

In some embodiments, the spacer unit comprises

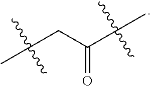 [SMILES: *CC(*)=O]

In some embodiments, the spacer unit comprises

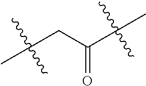 [SMILES: *CC(*)=O]

and (PEG)₂. In some embodiments, the spacer unit comprises

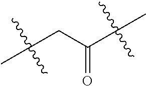 [SMILES: *CC(*)=O]

conjugated to (PEG)₂. In some embodiments, the spacer unit comprises

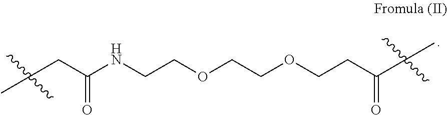 [SMILES: *CC(=O)NCCOCCOCCC(*)=O]

In some embodiments, linkers disclosed herein may be used in L-D constructs with other D moieties. In some embodiments, using a linker comprising a spacer unit comprising Formula (II) may provide benefits for various D moieties, including, e.g., improved conjugation stability, improved plasma stability, and/or in vivo anti-tumor activity compared to other linkers comprising alternative spacer units. In some embodiments, without being bound by theory, benefits of using a linker comprising Formula (II) with a STING agonist disclosed herein, e.g., a compound of Table 13, e.g., Compound 1, may include improved conjugation stability, improved plasma stability, and/or in vivo anti-tumor activity. In some embodiments, a linker comprising Formula (II) and a payload comprising a STING agonist disclosed herein, e.g., a compound of Table 13 demonstrates superior properties when conjugated to an anti-CD73 antibody disclosed herein. Exemplary evidence of the superior benefits of such L-D and antibody-drug conjugates are shown in Examples 4, 5, and 7.

A spacer unit may be used, for example, to link the antibody moiety to the drug moiety, either directly or indirectly. In some embodiments, the spacer unit links the antibody moiety to the drug moiety directly. In some embodiments, the antibody moiety and the drug moiety are attached via a spacer unit comprising one or more alkyl moieties (e.g., (CH₂)₃, or (CH₂)₄, or (CH₂)₅). In some embodiments, the antibody moiety and the drug moiety are attached via a spacer unit comprising one or more PEG moieties (e.g., (PEG)₂ or (PEG)₃ or (PEG)₄). In some embodiments, the antibody moiety and the drug moiety are attached via a spacer unit comprising Formula (II). In some embodiments, the spacer unit links the antibody moiety to the drug moiety indirectly. In some embodiments, the spacer unit links the antibody moiety to the drug moiety indirectly through a cleavable moiety (e.g., a cleavable peptide) and/or an antibody attachment moiety to join the spacer unit to the antibody moiety, e.g., a maleimide moiety or a carbobenzoxy-L-glutaminyl-glycine moiety.

In some embodiments, the spacer unit attaches to the antibody moiety (i.e., the antibody or antigen-binding fragment) via a maleimide moiety (Mal). A spacer unit that attaches to the antibody or antigen-binding fragment via a Mal is referred to herein as a “Mal-spacer unit.” In some embodiments, the Mal-spacer unit is reactive with a cysteine residue on the antibody or antigen-binding fragment. In some embodiments, the Mal-spacer unit is joined to the antibody or antigen-binding fragment via the cysteine residue. In some embodiments, the Mal-spacer unit comprises a PEG moiety. In some embodiments, the Mal-spacer unit comprises an alkyl moiety. In some embodiments, the Mal-spacer unit comprises Formula (II). In some embodiments, the spacer unit attaches to the antibody moiety (i.e., the antibody or antigen-binding fragment) via a MC moiety. A spacer unit that attaches to the antibody or antigen-binding fragment via an MC is referred to herein as an “MC-spacer unit.” In some embodiments, the MC-spacer unit is reactive with a cysteine residue on the antibody or antigen-binding fragment. In some embodiments, the MC-spacer unit is joined to the antibody or antigen-binding fragment via the cysteine residue. In some embodiments, the MC-spacer unit comprises a PEG moiety. In some embodiments, the MC-spacer unit comprises an alkyl moiety. In some embodiments, the MC-spacer unit comprises Formula (II).

In some embodiments, the linker comprises the Mal-spacer unit or MC-spacer unit and a cleavable peptide moiety. In some embodiments, the cleavable peptide moiety comprises an amino acid unit. In some embodiments, the amino acid unit comprises Val-Cit. In some embodiments, the amino acid unit comprises Val-Ala. In some embodiments, the linker comprises the Mal-spacer unit or MC-spacer unit and an amino acid unit. In some embodiments, the linker comprises Mal-(CH₂)n and an amino acid unit, where n is 3 to 5, or 3, 4, or 5. In some embodiments, the linker comprises MC—(CH₂)n and an amino acid unit, where n is 3 to 5, or 3, 4, or 5.

In some embodiments, the linker comprises Mal-(PEG)m and an amino acid unit, where m is 2 to 4, or 2, 3, or 4. In some embodiments, the linker comprises MC-(PEG)m and an amino acid unit, where m is 2 to 4, or 2, 3, or 4. In some embodiments, the amino acid unit comprises a cleavable dipeptide, e.g., Val-Cit or Val-Ala. In some embodiments, the linker comprises Mal-(PEG)ₙ-Val-Cit, where n is any integer between 1 and 10. In some embodiments, the linker comprises Mal-(PEG)ₙ-Val-Ala, where n is any integer between 1 and 10. In some embodiments, the linker comprises MC-(PEG)ₙ-Val-Cit, where n is any integer between 1 and 10. In some embodiments, the linker comprises MC-(PEG)ₙ-Val-Ala, where n is any integer between 1 and 10.

In some embodiments, the linker comprises Mal-Formula (II) and an amino acid unit. In some embodiments, the amino acid unit comprises a cleavable dipeptide, e.g., Val-Cit or Val-Ala. In some embodiments, the linker comprises Mal-Formula (II)-Val-Cit. In some embodiments, the linker comprises Mal-Formula (II)-Val-Ala.

In some embodiments, the Mal-spacer unit or MC-spacer unit attaches the antibody moiety (i.e., the antibody or antigen-binding fragment) to the cleavable moiety in the linker. In some embodiments, the Mal-spacer unit or MC-spacer unit attaches the antibody or antigen-binding fragment to a cleavable peptide moiety. In some embodiments, the cleavable peptide moiety comprises an amino acid unit. In some embodiments, the linker comprises Mal-spacer unit-amino acid unit. In some embodiments, the Mal-spacer unit comprises a PEG moiety. In some embodiments, the Mal-spacer-unit comprises an alkyl moiety. In some embodiments, the Mal-spacer unit comprises Formula (II). In some embodiments, the linker comprises MC-spacer unit-amino acid unit. In some embodiments, the MC-spacer unit comprises a PEG moiety. In some embodiments, the MC-spacer unit comprises an alkyl moiety.

In various embodiments, the cleavable moiety in the linker is joined directly to the drug moiety and/or to the antibody moiety. In other embodiments, a spacer unit is used to attach the cleavable moiety in the linker to the drug moiety and/or to the antibody moiety. In various embodiments, the drug moiety may be any STING agonist drug moiety disclosed herein, e.g., a compound disclosed in Table 13, infra. In various embodiments, the drug moiety is attached to the cleavable moiety in the linker by a spacer unit. In various embodiments, the drug moiety is Compound 1. In various embodiments, the Compound 1 moiety is attached to the cleavable moiety in the linker by a spacer unit. In some embodiments, the drug moiety, e.g., Compound 1, is attached to the cleavable moiety in the linker by a self-immolative unit. In some embodiments, the drug moiety, e.g., Compound 1, is attached to the cleavable moiety in the linker by a self-immolative unit, the cleavable moiety comprises an amino acid unit, and a further spacer unit, e.g., comprising one or more alkyl or PEG moieties or Formula (II), joins the cleavable moiety to the antibody moiety. In some embodiments, the drug moiety, e.g., Compound 1, is joined to an anti-CD73 antibody via a Mal-spacer unit in the linker joined to a cleavable peptide moiety and a pAB or pABC self-immolative unit. In some embodiments, the drug moiety, e.g., Compound 1, is joined to an anti-CD73 antibody via an MC-spacer unit in the linker joined to a cleavable peptide moiety and a pAB or pABC self-immolative unit.

A spacer unit may be “self-immolative” or “non-self-immolative.” A “non-self-immolative” spacer unit is one in which part or all of the spacer unit remains bound to the drug moiety upon cleavage of the linker. Examples of non-self-immolative units include, but are not limited to, a glycine spacer unit and a glycine-glycine spacer unit. Non-self-immolative units may eventually degrade over time but do not readily release a linked native drug entirely under cellular conditions. A “self-immolative” unit comprises any structure that allows for release of the native drug moiety after administration to a subject, e.g., under intracellular conditions. A “native drug” is one where no part of the spacer unit or other chemical modification remains after cleavage/degradation of the spacer unit.

Self-immolation chemistry is known in the art and may be readily selected for the disclosed ADCs. In various embodiments, the spacer unit attaching the cleavable moiety in the linker to the drug moiety (e.g., Compound 1) is self-immolative, and undergoes self-immolation concurrently with or shortly before/after cleavage of the cleavable moiety under intracellular conditions.

In various embodiments, a linker disclosed herein may comprise at least one self-immolative unit. Any of the linkers disclosed herein may comprise a first self-immolative unit. The phrase “first self-immolative unit” may indicate a linker comprising one self-immolative unit or a linker comprising one or more self-immolative units. In some embodiments, a linker disclosed herein comprises a first self-immolative unit and a second self-immolative unit.

In certain embodiments, the at least one self-immolative unit in the linker comprises a p-aminobenzyl unit. In some embodiments, a p-aminobenzyl alcohol (pABOH) is attached to an amino acid unit or other cleavable moiety in the linker via an amide bond, and a carbamate, methylcarbamate, or carbonate is made between the pABOH and the drug moiety. See, e.g., Hamann et al. (2005) Expert Opin. Ther. Patents 15:1087-103. In some embodiments, the at least one self-immolative unit is or comprises p-aminobenzyl (pAB). In some embodiments, the at least one self-immolative unit is or comprises p-aminobenzyloxycarbonyl (pABC). Without being bound by theory, it is thought that the self-immolation of pAB or pABC involves a spontaneous 1,6-elimination reaction. See, e.g., Jain et al. (2015) Pharm Res 32:3526-40.

In various embodiments, the structure of the p-aminobenzyl (pAB) used in the disclosed ADCs is shown below:

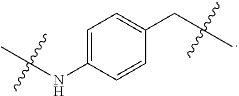 [SMILES: *Cc1ccc(N*)cc1]

In various embodiments, the structure of the p-aminobenzyloxycarbonyl (pABC) used in the disclosed ADCs is shown below:

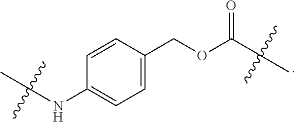 [SMILES: *Nc1ccc(COC(*)=O)cc1]

The structure of the pAB or pABC in a self-immolative unit may be substituted.

In some embodiments, the pAB is substituted with 1-3 substituents chosen from methyl, fluoro, chloro, trifluoromethyl, C₆-C₁₀ aryl, and C₅-C₁₂ heteroaryl. Exemplary substituted pAB units are disclosed in Table 11. In some embodiments, a linker disclosed herein may comprise a self-immolative unit selected from the self-immolative units disclosed in Table 11, infra.

TABLE 11
Exemplary Substituted pAB Moieties

| Substituted | | |
|---|---|---|
| pAB | Substituted | |
| Moiety | pAB Moiety | Structure |
| (N-Me)pAB | N-methyl-p- aminobenzyl | 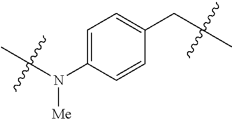 [SMILES: *Cc1ccc(N(*)C)cc1] |
| MAB | m-aminobenzyl | 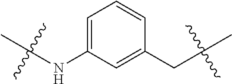 [SMILES: *Cc1cccc(N*)c1] |
| OAB | o-aminobenzyl | 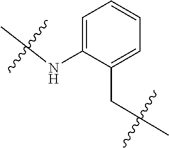 [SMILES: *Cc1ccccc1N*] |
| (2-Cl)pAB | 2-chloro-4- aminobenzyl | 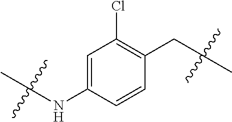 [SMILES: *Cc1ccc(N*)cc1Cl] |
| (3-Me)pAB | 3-methyl-4- aminobenzyl | 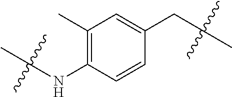 [SMILES: *Cc1ccc(N*)c(C)c1] |
| (3-Cl)pAB | 3-chloro-4- aminobenzyl | 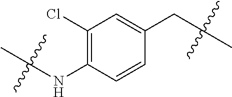 [SMILES: *Cc1ccc(N*)c(Cl)c1] |
| (3-F)pAB | 3-fluoro-4- aminobenzyl | 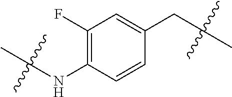 [SMILES: *Cc1ccc(N*)c(F)c1] |
| (2,5-Cl2)pAB | 2,5-dichloro- 4-aminobenzyl | 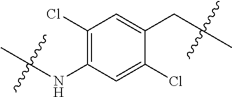 [SMILES: *Cc1cc(Cl)c(N*)cc1Cl] |
| (3-CF₃)pAB | 3-trifluoromethyl- 4-aminobenzyl | 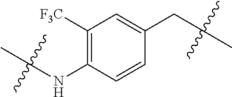 [SMILES: *Cc1ccc(N*)c(C(F)(F)F)c1] |
| (2-F)pAB | 2-fluoro-4- aminobenzyl | 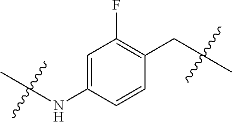 [SMILES: *Cc1ccc(N*)cc1F] |
| (3-MeO)pAB | 3-methoxy- 4-aminobenzyl | 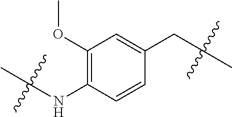 [SMILES: *Cc1ccc(N*)c(OC)c1] |
| (2,5-Me2)pAB | 2,5-dimethyl- 4-aminobenzyl | 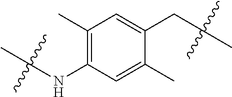 [SMILES: *Cc1cc(C)c(N*)cc1C] |
| (2,6-Cl2)pAB | 2,6-dichloro- 4-aminobenzyl | 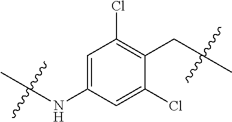 [SMILES: *Cc1c(Cl)cc(N*)cc1Cl] |
| (2-Me)pAB | 2-methyl- 4-aminobenzyl | 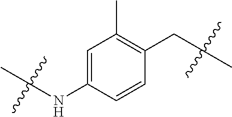 [SMILES: *Cc1ccc(N*)cc1C] |
| (2Py)pAB | (5-aminopyridin- 2-yl)methyl | 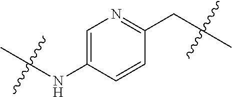 [SMILES: *Cc1ccc(N*)cn1] |

Linker moieties may be modified to achieve desirable properties of an ADC, e.g., stability, tolerability, and/or efficacy. For example, a linker comprising a modified pAB or pABC moiety may increase ADC stability and/or in vivo ADC tolerability (as determined by, for example, percent body weight loss) while minimizing reduced ADC efficacy when compared to a linker comprising pAB or pABC. Certain additional modifications to the linker-drug structure, e.g., spacer units or modified drug moiety attachment points, may be required to obtain one or more (e.g., all of these) properties. For instance, certain modifications or combinations of modifications may need to be made to enhance ADC stability while avoiding loss of efficacy. For example, in some embodiments, an ADC comprising LP2, LP16, LP20, LP54, or LP55 may achieve desirable properties of an ADC, e.g., stability, tolerability, and/or efficacy when compared to other anti-CD73 ADCs.

In some embodiments, any of the linkers disclosed herein may comprise a further self-immolative unit. In some embodiments, the further self-immolative unit attaches the first self-immolative unit to the drug moiety (e.g., Compound 1). The addition of one or more further self-immolative unit(s) to a linker-payload conjugate as disclosed herein may provide superior technical benefits, e.g., superior stability and/or improved activity, compared to other linker-payload conjugates comprising any of the payload compounds disclosed herein. Any of the linkers disclosed herein may comprise a second self-immolative unit.

Exemplary additional self-immolative units are disclosed in Table 12. In some embodiments, a linker-payload conjugate comprises a second self-immolative unit listed in Table 12, infra. In some embodiments, a linker-payload conjugate comprises Val-Ala-pAB and a second self-immolative unit selected from Table 12. In some embodiments, a linker-payload conjugate comprises Val-Ala-pABC and a second self-immolative unit selected from Table 12. In some embodiments, a linker-payload conjugate comprises Val-Cit-pAB and a second self-immolative unit selected from Table 12. In some embodiments, a linker-payload conjugate comprises Val-Cit-pABC and a second self-immolative unit selected from Table 12.

TABLE 12
Exemplary Self-Immolative Units

| Second Self- | | |
|---|---|---|
| immolative Unit | Chemical Name | Chemical Structure |
| Unit 1 (MEC) | (N-methylamino)ethoxycarbonyl | 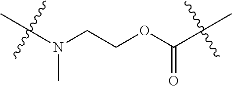 [SMILES: *C(=O)OCCN(*)C] |
| Unit 2 | 2-(N-methyl)aminomethylbenzoate | 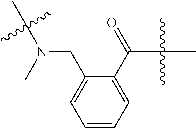 [SMILES: *C(=O)c1ccccc1CN(*)C] |
| Unit 3 | prolinolcarbonyl | 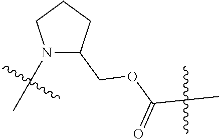 [SMILES: *C(=O)OCC1CCCN1*] |
| Unit 4 | N-methylamino-(cyclopropyl)-1- methoxycarbonyl | 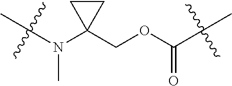 [SMILES: *C(=O)OCC1(N(*)C)CC1] |
| Unit 5 | (N-methylamino)-1,1-dimethyl-ethoxycarbonyl | 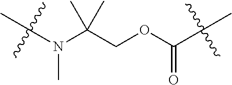 [SMILES: *C(=O)OCC(C)(C)N(*)C] |
| Unit 6 | N-methylamino-(cyclopropyl)-2- methoxycarbonyl | 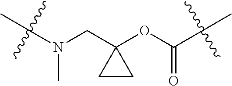 [SMILES: *C(=O)OC1(CN(*)C)CC1] |
| Unit 7 | (N-H)ethoxycarbonyl | 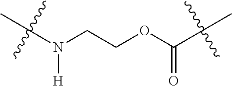 [SMILES: *NCCOC(*)=O] |
| Unit 8 | (N-methylamino)-2- cyclopropyl]methoxycarbonyl | 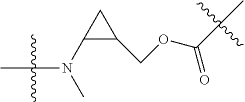 [SMILES: *C(=O)OCC1CC1N(*)C] |
| Unit 9 | 3-(N-methylamino)butanoyl | 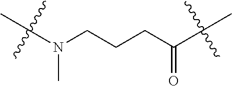 [SMILES: *C(=O)CCCN(*)C] |
| Unit 10 | 4-monofluoro-prolinolcarbonyl | 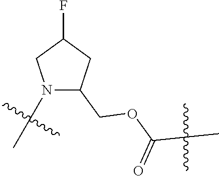 [SMILES: *C(=O)OCC1CC(F)CN1*] |
| Unit 11 | 4,4-difluoroprolinolcarbonyl | 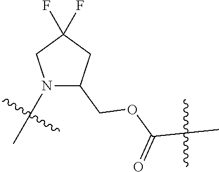 [SMILES: *C(=O)OCC1CC(F)(F)CN1*] |
| Unit 12 | 3-monofluoro-prolinolcarbonyl | 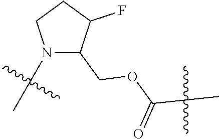 [SMILES: *C(=O)OCC1C(F)CCN1*] |
| Unit 13 | 3,3-difluoroprolinolcarbonyl | 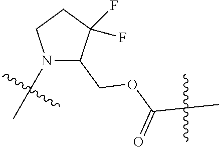 [SMILES: *C(=O)OCC1N(*)CCC1(F)F] |

Units 3, 8, and 10-13 include all stereoisomers.

In some embodiments, the further self-immolative unit comprises a Unit 1 moiety. In some embodiments, a Unit 1 moiety attaches the first self-immolative unit to the drug moiety (e.g., Compound 1) (“self-immolative unit-Unit 1 moiety”). In some embodiments, the further self-immolative unit comprises a Unit 2 moiety. In some embodiments, a Unit 2 moiety attaches the first self-immolative unit to the drug moiety (e.g., Compound 1) (“self-immolative unit-Unit 2 moiety”). In some embodiments, the further self-immolative unit comprises a Unit 3 moiety. In some embodiments, a Unit 3 moiety attaches the first self-immolative unit to the drug moiety (e.g., Compound 1) (“self-immolative unit-Unit 3 moiety”). In some embodiments, the further self-immolative unit comprises a Unit 4 moiety. In some embodiments, a Unit 4 moiety attaches the first self-immolative unit to the drug moiety (e.g., Compound 1) (“self-immolative unit-Unit 4 moiety”). In some embodiments, the further self-immolative unit comprises a Unit 5 moiety. In some embodiments, a Unit 5 moiety attaches the first self-immolative unit to the drug moiety (e.g., Compound 1) (“self-immolative unit-Unit 5 moiety”). In some embodiments, the further self-immolative unit comprises a Unit 6 moiety. In some embodiments, a Unit 6 moiety attaches the first self-immolative unit to the drug moiety (e.g., Compound 1) (“self-immolative unit-Unit 6 moiety”). In some embodiments, the further self-immolative unit comprises a Unit 7 moiety. In some embodiments, a Unit 7 moiety attaches the first self-immolative unit to the drug moiety (e.g., Compound 1) (“self-immolative unit-Unit 7 moiety”). In some embodiments, the further self-immolative unit comprises a Unit 8 moiety. In some embodiments, a Unit 8 moiety attaches the first self-immolative unit to the drug moiety (e.g., Compound 1) (“self-immolative unit-Unit 8 moiety”). In some embodiments, the further self-immolative unit comprises a Unit 9 moiety. In some embodiments, a Unit 9 moiety attaches the first self-immolative unit to the drug moiety (e.g., Compound 1) (“self-immolative unit-Unit 9 moiety”). In some embodiments, the further self-immolative unit comprises a Unit 10 moiety. In some embodiments, a Unit 10 moiety attaches the first self-immolative unit to the drug moiety (e.g., Compound 1) (“self-immolative unit-Unit 10 moiety”). In some embodiments, the further self-immolative unit comprises a Unit 11 moiety. In some embodiments, a Unit 11 moiety attaches the first self-immolative unit to the drug moiety (e.g., Compound 1) (“self-immolative unit-Unit 11 moiety”). In some embodiments, the further self-immolative unit comprises a Unit 12 moiety. In some embodiments, a Unit 12 moiety attaches the first self-immolative unit to the drug moiety (e.g., Compound 1) (“self-immolative unit-Unit 12 moiety”). In some embodiments, the further self-immolative unit comprises a Unit 13 moiety. In some embodiments, a Unit 13 moiety attaches the first self-immolative unit to the drug moiety (e.g., Compound 1) (“self-immolative unit-Unit 13 moiety”).

In various embodiments, a cleavable moiety in a linker attaches directly or indirectly to a sulfur in the drug moiety. The drug moiety may be any suitable drug moiety disclosed herein, e.g., a compound disclosed in Table 13, infra. In some embodiments, the drug moiety is or comprises Compound 1. In some embodiments, the cleavable moiety in the linker attaches directly or indirectly to the S-14 sulfur in a STING agonist drug moiety disclosed herein (e.g., Compound 1). In some embodiments, the one or more self-immolative unit(s) comprises pAB. In some embodiments, the pAB attaches the cleavable moiety in the linker to the S-14 sulfur in a STING agonist drug moiety disclosed herein (e.g., Compound 1). In some embodiments, the pAB undergoes self-immolation upon cleavage of the cleavable moiety, and the STING agonist drug moiety (e.g., Compound 1) is released from the ADC in its native, active form. In some embodiments, the cleavable moiety comprises an amino acid unit. In some embodiments, the linker comprises amino acid unit-pAB. In some embodiments, the amino acid unit is Val-Cit. In some embodiments, the linker comprises Val-Cit-pAB. In some embodiments, the amino acid unit is Val-Ala. In some embodiments, the linker comprises Val-Ala-pAB.

In various embodiments, a cleavable moiety in the linker attaches directly or indirectly to a nitrogen in the drug moiety. The drug moiety may be a STING agonist drug moiety disclosed herein, e.g., a compound disclosed in Table 13, infra. In some embodiments, the drug moiety is or comprises Compound 1. In some embodiments, the nitrogen in the STING agonist drug moiety (e.g., Compound 1) is the N-34 nitrogen. In some embodiments, the nitrogen in the STING agonist drug moiety (e.g., Compound 1) is the N-39 nitrogen. In some embodiments, the one or more self-immolative unit(s) comprises pAB. In some embodiments, the one or more self-immolative unit(s) comprises pABC. In some embodiments, the one or more self-immolative unit(s) comprises a Unit 1 moiety. In some embodiments, the one or more self-immolative unit(s) comprises pABC-Unit 1 moiety. In some embodiments, the carboxylate moiety of the pABC is bound to the n-methyl moiety of the Unit 1 to form an N-methylcarbamate moiety. In some embodiments, the one or more self-immolative unit(s) comprises a Unit 2 moiety. In some embodiments, the one or more self-immolative unit(s) comprises pABC-Unit 2 moiety. In some embodiments, the one or more self-immolative unit(s) comprises a Unit 3 moiety. In some embodiments, the one or more self-immolative unit(s) comprises pABC-Unit 3 moiety.

In some embodiments, the linker comprises a third spacer unit between the first spacer unit and the second spacer unit. In some embodiments, the second and/or third spacer unit is selected from a moiety of Table 12, supra. In some embodiments, the linker comprises a third spacer unit between the pABC spacer unit and the Unit 1 spacer unit. In some embodiments, the linker comprises a third spacer unit between the pABC spacer unit and the Unit 2 spacer unit. In some embodiments, the linker comprises a third spacer unit between the pABC spacer unit and the Unit 3 spacer unit. In some embodiments, the pABC attaches the cleavable moiety in the linker to the N-34 nitrogen in Compound 1. In some embodiments, the pABC attaches the cleavable moiety in the linker to the N-39 nitrogen in Compound 1.

In some embodiments, the pABC-Unit 1 moiety attaches the cleavable moiety in the linker to the N-34 nitrogen in Compound 1. In some embodiments, the pABC-Unit 1 moiety attaches the cleavable moiety in the linker to the N-39 nitrogen in Compound 1. In some embodiments, the pABC or pABC-Unit 1 moiety undergoes self-immolation upon cleavage of the cleavable moiety, and Compound 1 is released from the ADC in its native, active form. In some embodiments, the release of Compound 1 from the antibody and linker occurs in a stepwise fashion, wherein first the cleavable moiety in the linker is cleaved, then the pABC moiety undergoes self-immolation, and then the Unit 1 moiety undergoes self-immolation. In some embodiments, the cleavable moiety comprises an amino acid unit. In some embodiments, the linker comprises amino acid unit-pABC. In some embodiments, the linker comprises amino acid unit-pABC-Unit 1 moiety. In some embodiments, the amino acid unit is Val-Cit. In some embodiments, the linker comprises Val-Cit-pABC. In some embodiments, the linker comprises Val-Cit-pABC-Unit 1 moiety. In some embodiments, the amino acid unit is Val-Ala. In some embodiments, the linker comprises Val-Ala-pABC. In some embodiments, the linker comprises Val-Ala-pABC-Unit 1 moiety.

In some embodiments, the pABC-Unit 2 moiety attaches the cleavable moiety in the linker to the N-34 nitrogen in Compound 1. In some embodiments, the pABC-Unit 2 moiety attaches the cleavable moiety in the linker to the N-39 nitrogen in Compound 1. In some embodiments, the pABC or pABC-Unit 8 moiety undergoes self-immolation upon cleavage of the cleavable moiety, and Compound 1 is released from the ADC in its native, active form. In some embodiments, the release of Compound 1 from the antibody and linker occurs in a stepwise fashion, wherein first the cleavable moiety in the linker is cleaved, then the pABC moiety undergoes self-immolation, and then the Unit 2 moiety undergoes self-immolation. In some embodiments, the cleavable moiety comprises an amino acid unit. In some embodiments, the linker comprises amino acid unit-pABC. In some embodiments, the linker comprises amino acid unit-pABC-Unit 2 moiety. In some embodiments, the amino acid unit is Val-Cit. In some embodiments, the linker comprises Val-Cit-pABC. In some embodiments, the linker comprises Val-Cit-pABC-Unit 2 moiety. In some embodiments, the amino acid unit is Val-Ala. In some embodiments, the linker comprises Val-Ala-pABC. In some embodiments, the linker comprises Val-Ala-pABC-Unit 2 moiety.

In some embodiments, the pABC-Unit 3 moiety attaches the cleavable moiety in the linker to the N-34 nitrogen in Compound 1. In some embodiments, the pABC-Unit 3 moiety attaches the cleavable moiety in the linker to the N-39 nitrogen in Compound 1. In some embodiments, the pABC or pABC-Unit 3 moiety undergoes self-immolation upon cleavage of the cleavable moiety, and Compound 1 is released from the ADC in its native, active form. In some embodiments, the release of Compound 1 from the antibody and linker occurs in a stepwise fashion, wherein first the cleavable moiety in the linker is cleaved, then the pABC moiety undergoes self-immolation, and then the Unit 3 moiety undergoes self-immolation. In some embodiments, the cleavable moiety comprises an amino acid unit. In some embodiments, the linker comprises amino acid unit-pABC. In some embodiments, the linker comprises amino acid unit-pABC-Unit 3 moiety. In some embodiments, the amino acid unit is Val-Cit. In some embodiments, the linker comprises Val-Cit-pABC. In some embodiments, the linker comprises Val-Cit-pABC-Unit 3 moiety. In some embodiments, the amino acid unit is Val-Ala. In some embodiments, the linker comprises Val-Ala-pABC. In some embodiments, the linker comprises Val-Ala-pABC-Unit 3 moiety.

In some embodiments, the at least one self-immolative unit (e.g., pAB, pABC, pABC-Unit 1 moiety, pABC-Unit 2 moiety, or pABC-Unit 3 moiety) undergoes self-immolation upon cleavage of a cleavable peptide moiety in the linker. In some embodiments, the self-immolation of the at least one self-immolative unit (e.g., pAB, pABC, pABC-Unit 1 moiety, pABC-Unit 2 moiety, or pABC-Unit 3 moiety) occurs in a stepwise manner after cleavage of a cleavable peptide moiety in the linker, starting from the self-immolative moiety closest to the cleavable peptide moiety. In some embodiments, the at least one self-immolative unit (e.g., pAB, pABC, pABC-Unit 1 moiety, pABC-Unit 2 moiety, or pABC-Unit 3 moiety) undergoes self-immolation in a stepwise manner after cleavage of a cleavable peptide moiety in the linker, wherein the first self-immolative unit (e.g., pABC or pAB) undergoes self-immolation prior to self-immolation of the second self-immolative unit (e.g., Unit 1 moiety, Unit 2 moiety, or Unit 3 moiety). In some embodiments, the cleavable peptide moiety comprises an amino acid unit. In some embodiments, the linker comprises amino acid unit-pAB. In some embodiments, the linker comprises amino acid unit-pABC. In some embodiments, the linker comprises amino acid unit-pABC-Unit 1 moiety. In some embodiments, the linker comprises amino acid unit-pABC-Unit 2 moiety. In some embodiments, the linker comprises amino acid unit-pABC-Unit 3 moiety. In some embodiments, the amino acid unit is Val-Cit. In some embodiments, the linker comprises Val-Cit-pAB. In some embodiments, the linker comprises Val-Cit-pABC. In some embodiments, the linker comprises Val-Cit-pABC-Unit 1 moiety. In some embodiments, the linker comprises Val-Cit-pABC-Unit 2 moiety. In some embodiments, the linker comprises Val-Cit-pABC-Unit 3 moiety. In some embodiments, the amino acid unit is Val-Ala. In some embodiments, the linker comprises Val-Ala-pAB. In some embodiments, the linker comprises Val-Ala-pABC. In some embodiments, the linker comprises Val-Ala-pABC-Unit 1 moiety. In some embodiments, the linker comprises Val-Ala-pABC-Unit 2 moiety. In some embodiments, the linker comprises Val-Ala-pABC-Unit 3 moiety.

In various aspects, the antibody moiety of the ADC is conjugated to the drug moiety via a linker, wherein the linker comprises an MC-spacer unit, a cleavable amino acid unit, and a pAB. In some embodiments, the linker comprises MC-Val-Cit-pAB. In some embodiments, the linker comprises MC-Val-Ala-pAB.

In various aspects, the antibody moiety of the ADC is conjugated to the drug moiety via a linker, wherein the linker comprises an MC-spacer unit, a cleavable amino acid unit, and a pABC. In some embodiments, the linker comprises MC-Val-Cit-pABC. In some embodiments, the linker comprises MC-Val-Ala-pABC.

In various aspects, the antibody moiety of the ADC is conjugated to the drug moiety via a linker, wherein the linker comprises an MC unit, a cleavable amino acid unit, a pABC, and a Unit 1 moiety. In some embodiments, the linker comprises MC-Val-Cit-pABC-Unit 1 moiety. In some embodiments, the linker comprises MC-Val-Ala-pABC-Unit 1 moiety.

In various aspects, the antibody moiety of the ADC is conjugated to the drug moiety via a linker, wherein the linker comprises an MC unit, a cleavable amino acid unit, a pABC, and a Unit 2 moiety. In some embodiments, the linker comprises MC-Val-Cit-pABC-Unit 2 moiety. In some embodiments, the linker comprises MC-Val-Ala-pABC-Unit 2 moiety.

In various aspects, the antibody moiety of the ADC is conjugated to the drug moiety via a linker, wherein the linker comprises an MC unit, a cleavable amino acid unit, a pABC, and a Unit 3 moiety. In some embodiments, the linker comprises MC-Val-Cit-pABC-Unit 3 moiety. In some embodiments, the linker comprises MC-Val-Ala-pABC-Unit 3 moiety.

In some embodiments, the antibody moiety is conjugated to the drug moiety via a linker comprising an MC moiety and an amino acid. In some embodiments, the antibody moiety is conjugated to the drug moiety via a linker comprising a MC moiety, an amino acid, and a pAB. In some embodiments, the antibody moiety is conjugated to the drug moiety via a linker comprising a MC moiety, an amino acid, and a pABC. In some embodiments, the antibody moiety is conjugated to the drug moiety via a linker comprising a MC moiety, an amino acid, a pABC, and a Unit 1 moiety. In some embodiments, the antibody moiety is conjugated to the drug moiety via a linker comprising a MC moiety, an amino acid, a pABC, and a Unit 2 moiety. In some embodiments, the antibody moiety is conjugated to the drug moiety via a linker comprising a MC moiety, an amino acid, a pABC, and a Unit 3 moiety.

In various aspects, the antibody moiety of the ADC is conjugated to the drug moiety via a linker, wherein the linker comprises a Mal-spacer unit, a cleavable amino acid unit, and a pAB. In some embodiments, the linker comprises Mal-Formula (II)-Val-Cit-pAB. In some embodiments, the linker comprises Mal-Formula (II)-Val-Ala-pAB. In various aspects, the antibody moiety of the ADC is conjugated to the drug moiety via a linker, wherein the linker comprises a Mal-spacer unit, a cleavable amino acid unit, and a pABC. In some embodiments, the linker comprises Mal-Formula (II)-Val-Cit-pABC. In some embodiments, the linker comprises Mal-Formula (II)-Val-Ala-pABC.

In various aspects, the antibody moiety of the ADC is conjugated to the drug moiety via a linker, wherein the linker comprises a Mal unit, a cleavable amino acid unit, a pABC, and a Unit 1 moiety. In some embodiments, the linker comprises Mal-Formula (II)-Val-Cit-pABC-Unit 1 moiety. In some embodiments, the linker comprises Mal-Formula (II)-Val-Ala-pABC-Unit 1 moiety.

In various aspects, the antibody moiety of the ADC is conjugated to the drug moiety via a linker, wherein the linker comprises a Mal unit, a cleavable amino acid unit, a pABC, and a Unit 2 moiety. In some embodiments, the linker comprises Mal-Formula (II)-Val-Cit-pABC-Unit 2 moiety. In some embodiments, the linker comprises Mal-Formula (II)-Val-Ala-pABC-Unit 2 moiety.

In various aspects, the antibody moiety of the ADC is conjugated to the drug moiety via a linker, wherein the linker comprises a Mal unit, a cleavable amino acid unit, a pABC, and a Unit 3 moiety. In some embodiments, the linker comprises Mal-Formula (II)-Val-Cit-pABC-Unit 3 moiety. In some embodiments, the linker comprises Mal-Formula (II)-Val-Ala-pABC-Unit 3 moiety.

In some embodiments, the drug moiety is Compound 1.

In some embodiments, the drug moiety is Compound 2.

### Drug Moieties

The drug moiety (D) of the linker-drug conjugates and ADCs disclosed herein can be any chemotherapeutic agent. In some embodiments, the drug moiety is an immunostimulant. In some embodiments, the drug moiety is a Toll-like receptor (TLR) agonist. In some embodiments, the drug moiety is a STING agonist. Exemplary STING agonists are known in the art and include cyclic dinucleotides, e.g., macrocycle-bridged STING agonists, non-cyclic dinucleotides. In some embodiments, the drug moiety is a non-cyclic dinucleotide. In some embodiments, the drug moiety is a macrocycle-bridged STING agonist.

In some embodiments, the drug moiety of a linker-drug conjugate or ADC disclosed herein comprises a compound selected from:

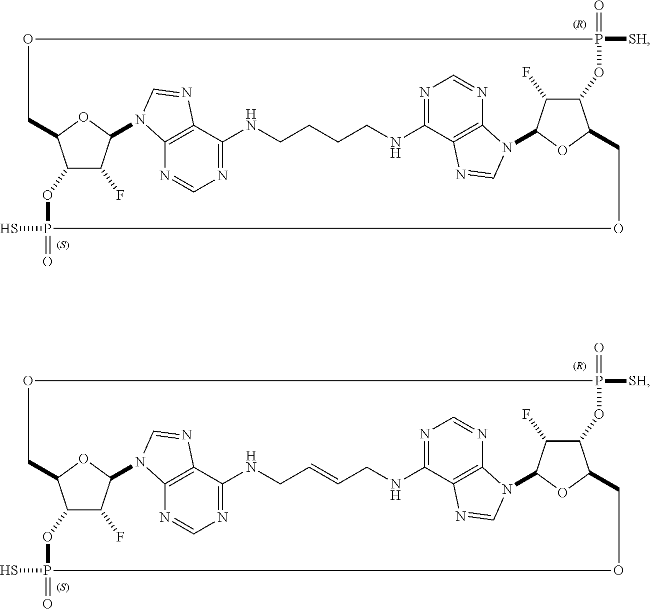

[symbol]

, and
and salts thereof.

In some embodiments, D comprises Compound 1. In some embodiments, D comprises Compound 2.

In some embodiments, a STING agonist in a linker-drug conjugate or ADC disclosed herein comprises Compound 1. The structure of Compound 1 is shown below:

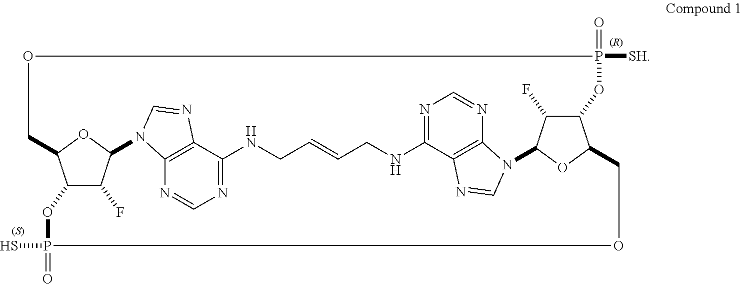

As noted above, the term Compound 1 as used herein also encompasses salts of the structure shown above unless context indicates otherwise. In some embodiments, the drug moiety is Compound 1. In some embodiments, a linker, e.g., the linker of an ADC, is attached to Compound 1 via the N-34 nitrogen on Compound 1. In some embodiments, a linker, e.g., the linker of an ADC, is attached to Compound 1 via the N-39 nitrogen on Compound 1. In some embodiments, the pAB is an analog of pAB as disclosed above. In some embodiments, the linker of the ADC covalently attaches to the N-34 nitrogen on Compound 1 via pABC. In some embodiments, the linker of the ADC covalently attaches to the N-39 nitrogen on Compound 1 via pABC. In some embodiments, the linker of the ADC covalently attaches to the N-34 nitrogen on Compound 1 via a second self immolative unit as disclosed below. In some embodiments, the linker of the ADC covalently attaches to the N-39 nitrogen on Compound 1 via a second self immolative unit as disclosed below.

In some embodiments, the STING agonist in a linker-drug conjugate or ADC disclosed herein comprises Compound 2. The structure of Compound 2 is shown below:

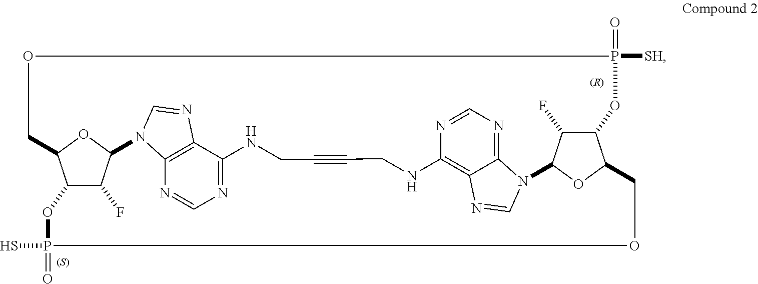

The term Compound as used herein also encompasses salts of the structure shown above unless context indicates otherwise. In some embodiments, the drug moiety is Compound 2. In some embodiments, a linker, e.g., the linker of an ADC, is attached to Compound 2 via the N-34 nitrogen on Compound 2. In some embodiments, a linker, e.g., the linker of an ADC, is attached to Compound 2 via the N-39 nitrogen on Compound 2. In some embodiments, the pAB is an analog of pAB as disclosed above. In some embodiments, the linker of the ADC covalently attaches to the N-34 nitrogen on Compound 2 via pABC. In some embodiments, the linker of the ADC covalently attaches to the N-39 nitrogen on Compound 2 via pABC. In some embodiments, the linker of the ADC covalently attaches to the N-34 nitrogen on Compound 2 via a second self immolative unit as disclosed below. In some embodiments, the linker of the ADC covalently attaches to the N-39 nitrogen on Compound 2 via a second self immolative unit as disclosed below.

In some embodiments, the STING agonist in a linker-drug conjugate or ADC disclosed herein comprises a compound selected from Table 13, infra, or is a salt thereof (unless context indicates otherwise).

TABLE 13
Exemplary STING agonist compounds

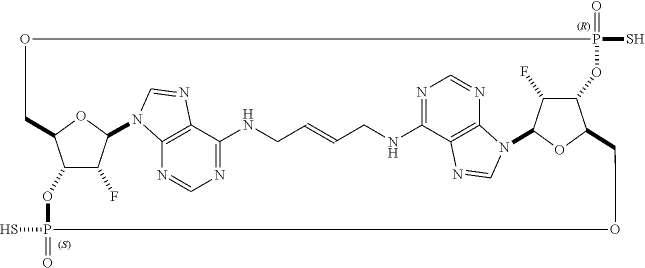

Compound 1

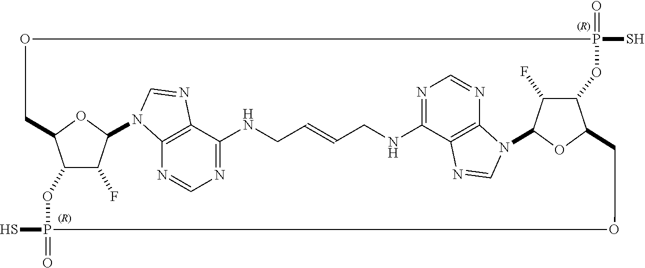

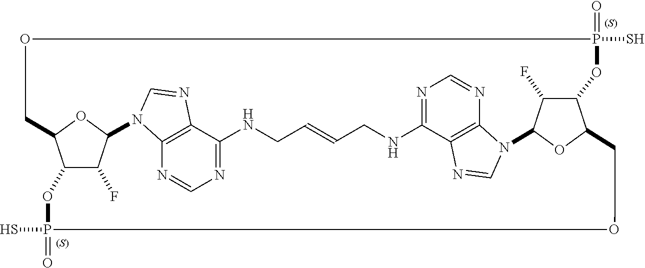

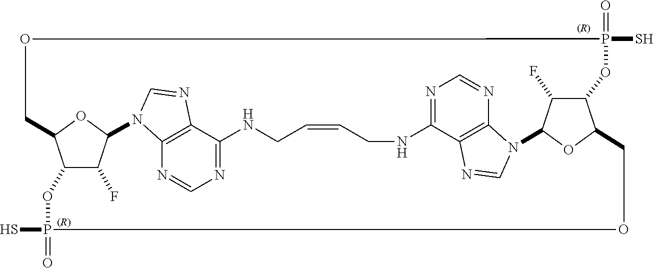

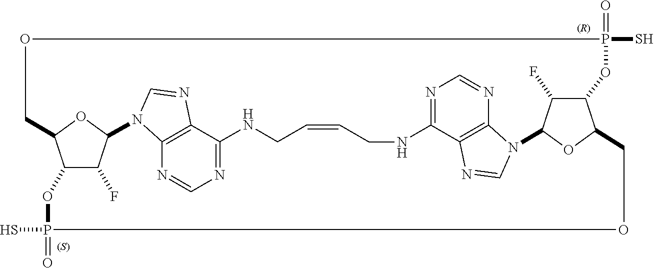

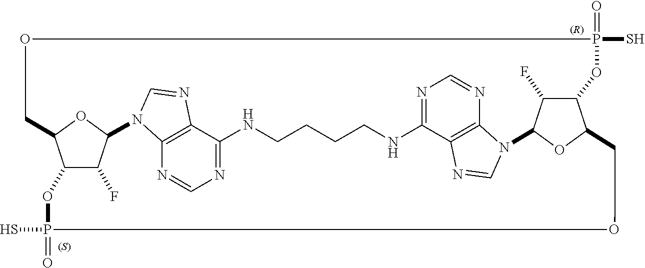

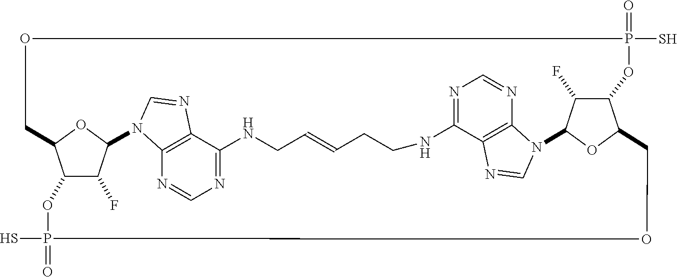 [SMILES: O=P1(S)OC[C@H]2O[C@@H]3[C@H](F)[C@@H]2OP(=O)(S)OC[C@H]2O[C@H]([C@H](F)[C@@H]2O1)n1cnc2c(ncnc21)NCC/C=C/CNc1ncnc2c1ncn23]

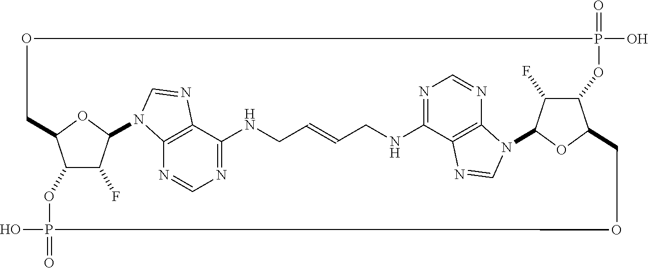 [SMILES: O=P1(O)OC[C@H]2O[C@@H]3[C@H](F)[C@@H]2OP(=O)(O)OC[C@H]2O[C@H]([C@H](F)[C@@H]2O1)n1cnc2c(ncnc21)NC/C=C/CNc1ncnc2c1ncn23]

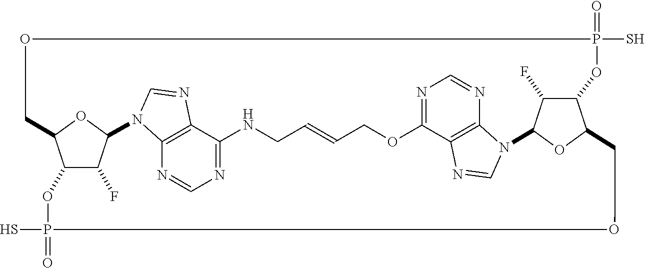

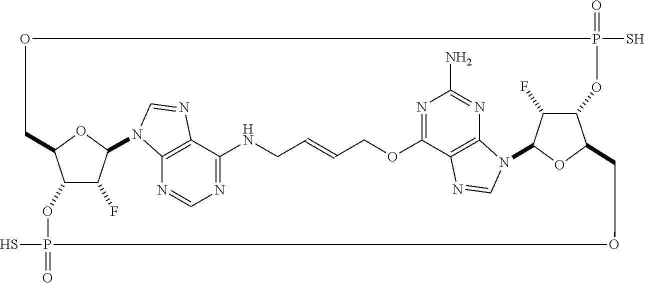

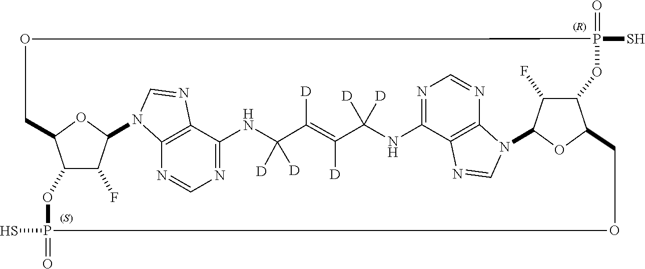

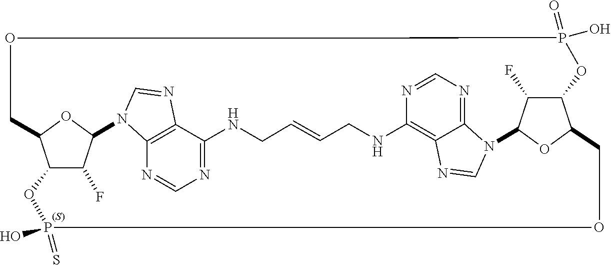

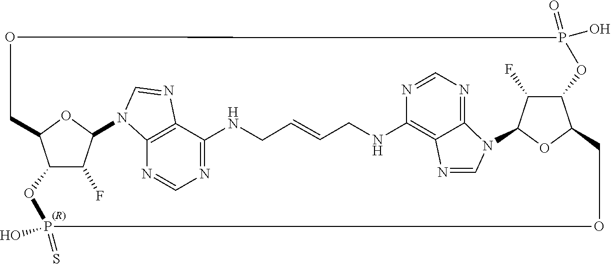

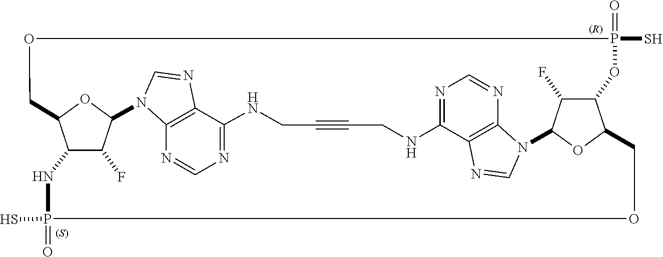

Compound 2

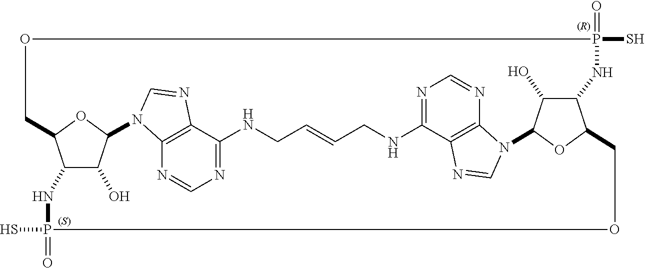

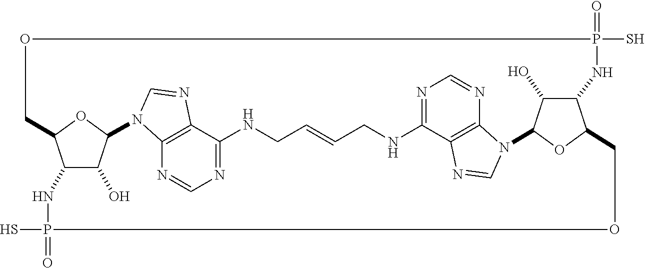

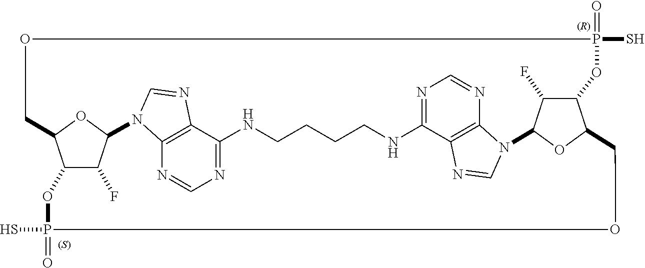

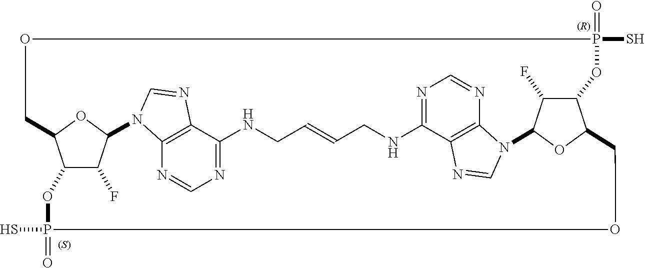

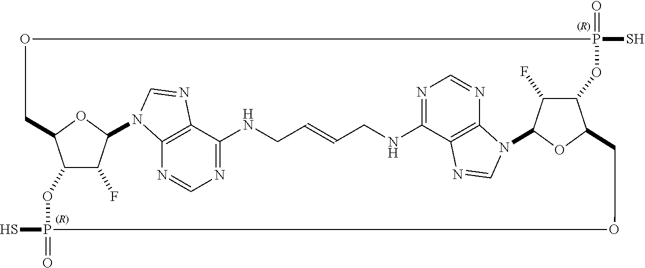

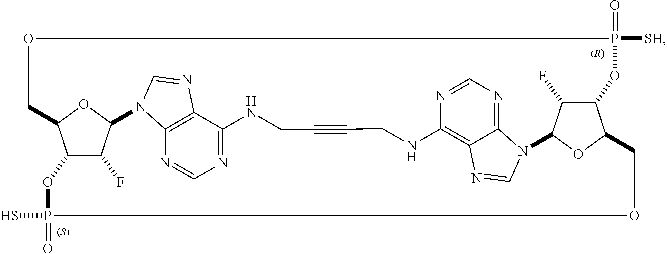

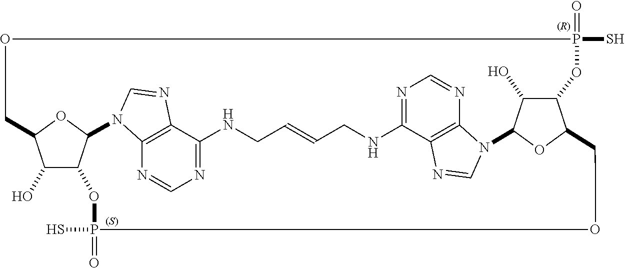

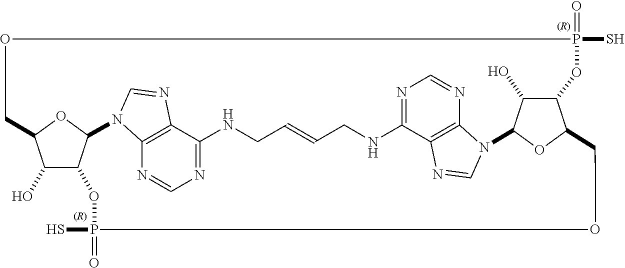

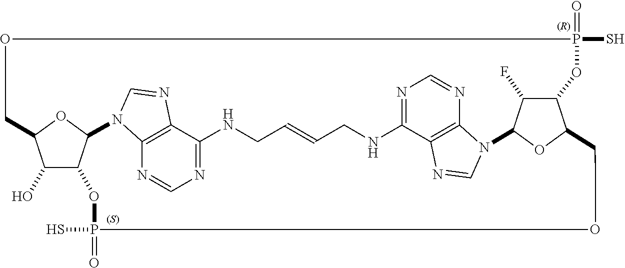

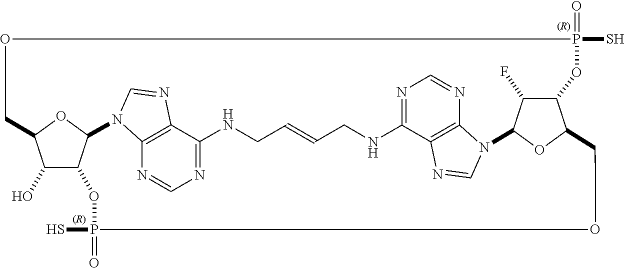

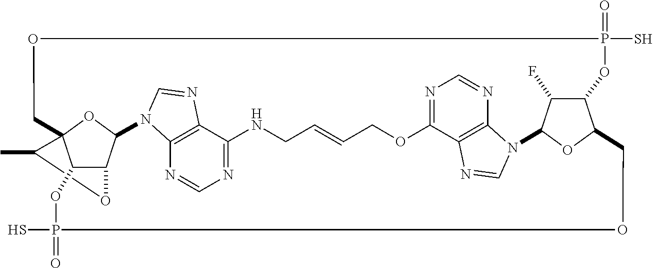

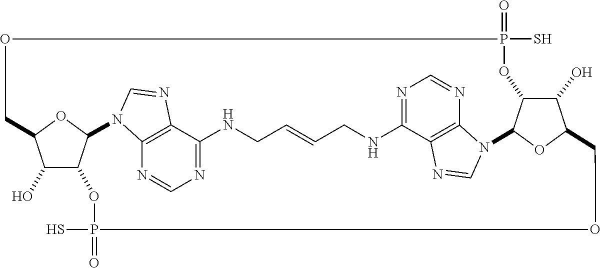

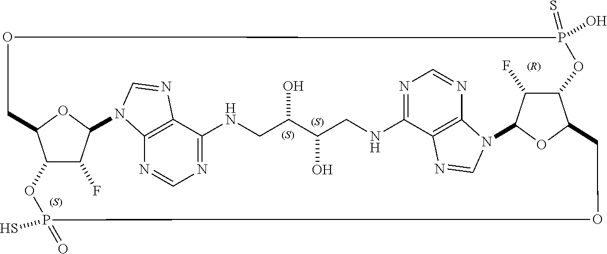

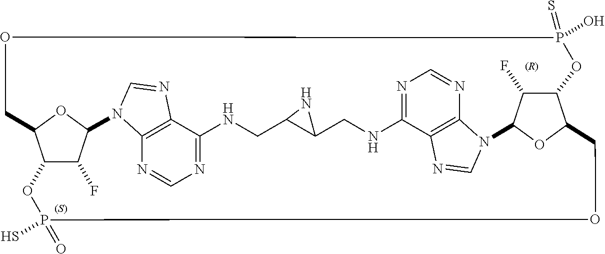

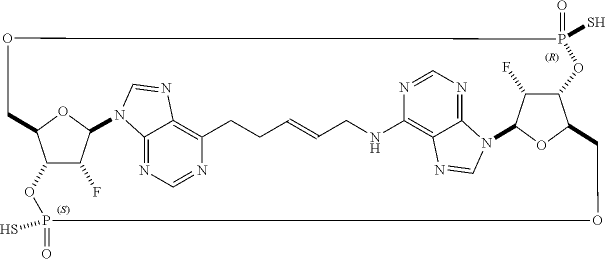

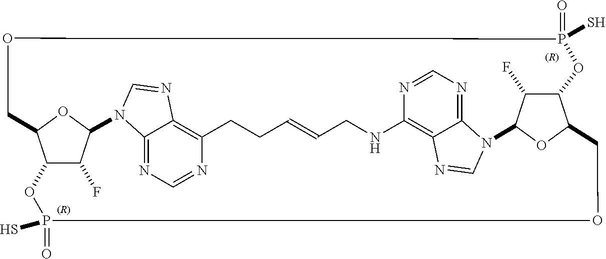

Isomers of compounds of Table 13, deuterated derivatives of the compounds and isomers; and salts of the compounds, isomers, and deuterated derivatives may also be used in a linker-drug conjugate or ADC disclosed herein.

In certain embodiments, an intermediate, such as a precursor of a linker disclosed above, is reacted with the drug moiety under appropriate conditions. In certain embodiments, reactive groups are used on the drug and/or the intermediate or linker. The product of the reaction between the drug and the intermediate, or the derivatized drug, is subsequently reacted with the antibody or antigen-binding fragment under appropriate conditions, e.g., according to the methods discussed below. Alternatively, the linker or intermediate may first be reacted with the antibody or a derivatized antibody, and then reacted with the drug or derivatized drug.

A number of different reactions are available for covalent attachment of drugs and/or linkers to the antibody moiety. This is often accomplished by reaction of one or more amino acid residues of the antibody molecule, including the amine groups of lysine, the free carboxylic acid groups of glutamic acid and aspartic acid, the sulfhydryl groups of cysteine, and the various moieties of the aromatic amino acids. For instance, non-specific covalent attachment may be undertaken using a carbodiimide reaction to link a carboxy (or amino) group on a compound to an amino (or carboxy) group on an antibody moiety. Additionally, bifunctional agents such as dialdehydes or imidoesters may also be used to link the amino group on a compound to an amino group on an antibody moiety. Also available for attachment of drugs to binding agents is the Schiff base reaction. This method involves the periodate oxidation of a drug that contains glycol or hydroxy groups, thus forming an aldehyde which is then reacted with the binding agent. Attachment occurs via formation of a Schiff base with amino groups of the binding agent. Isothiocyanates may also be used as coupling agents for covalently attaching drugs to binding agents. Other techniques are known to the skilled artisan and within the scope of the present disclosure.

### Linker-Drug Conjugates

The present disclosure provides linker-drug conjugates comprising L-D, wherein L is a cleavable linker that covalently attaches to D. The terms “linker-drug conjugate” and “linker-payload conjugate” are used interchangeably herein. The linker-drug conjugates disclosed herein are suitable for conjugation to a variety of antibodies, including anti-CD73 antibodies disclosed herein. In the L-D context, D is a compound that forms a covalent bond with L, which results in the loss of at least one hydrogen radical. In the L-D context, D may be any suitable compound that would benefit from a disclosed linker. In some embodiments, D is selected from any of the compounds disclosed herein. In the L-D context, L may be selected from any linker disclosed herein. In some embodiments, D is selected from a compound of Table 13 or a salt thereof.

In some embodiments, L is attached to D via a bridge nitrogen atom. In some embodiments, L is attached to D at the N-34 nitrogen or the N-39 nitrogen. In some embodiments, L is attached to D at the N-34 nitrogen. In some embodiments, L is attached to D at the N-39 nitrogen.

In some embodiments, D comprises a compound of Table 13 or a salt thereof. Exemplary compounds are shown below. In some embodiments, D comprises a compound of Formula (III) selected from:

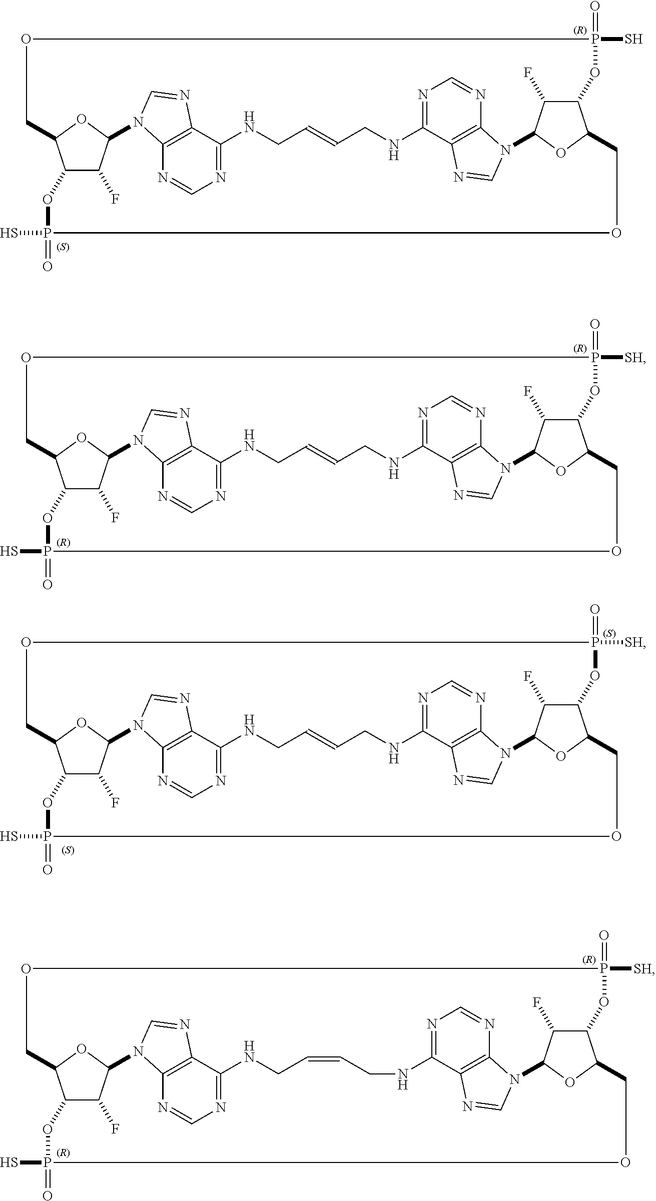

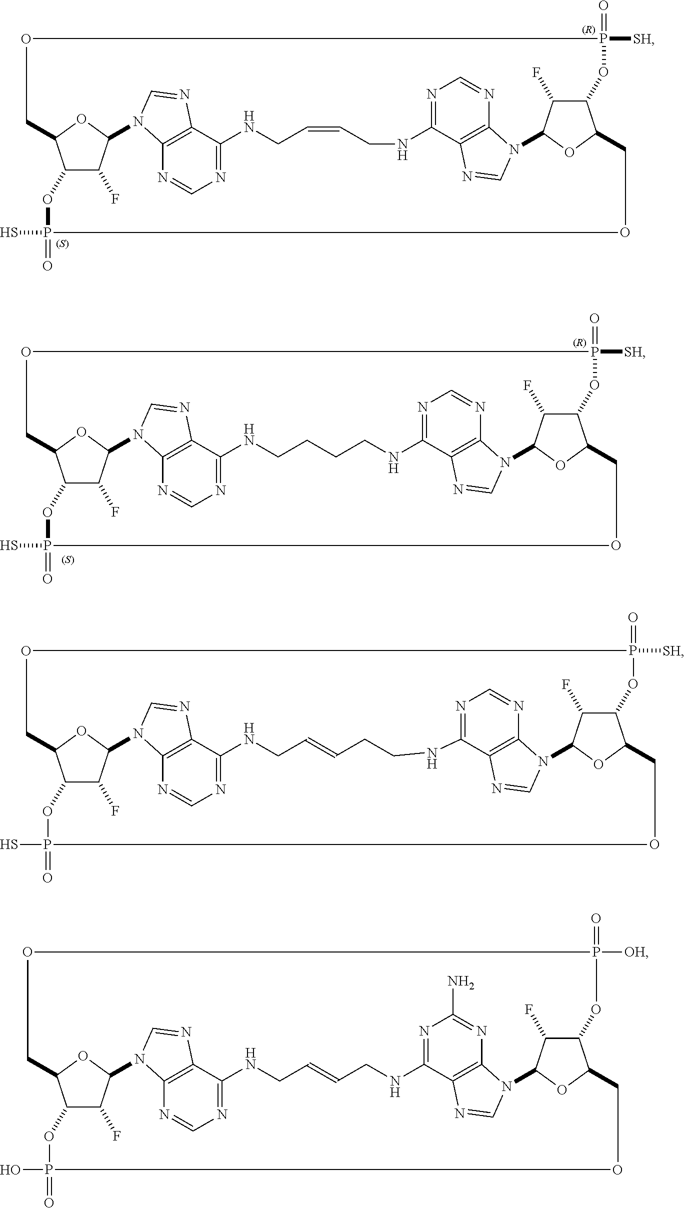

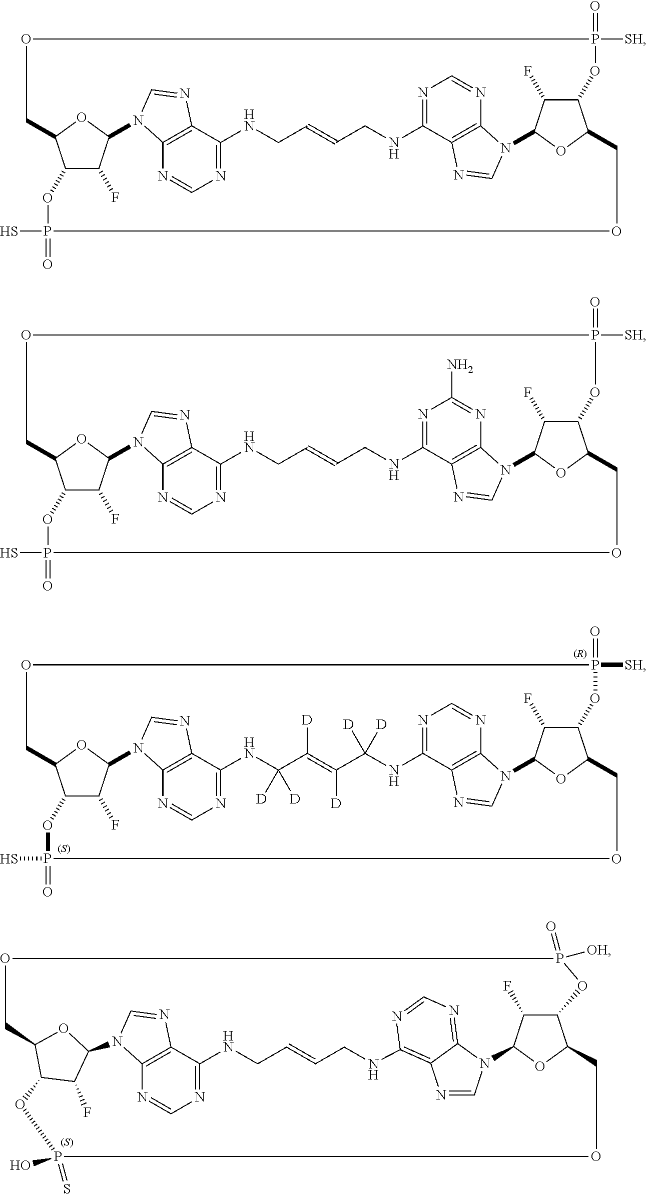

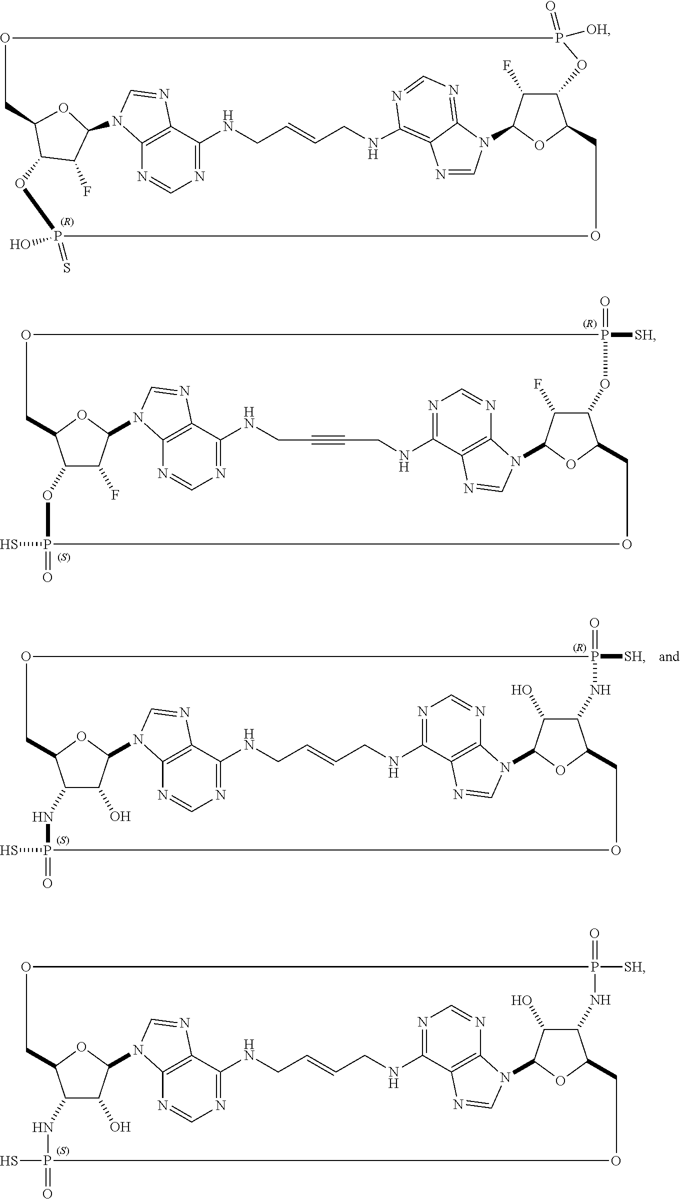

and salts thereof.

In some embodiments, the compound is selected from:

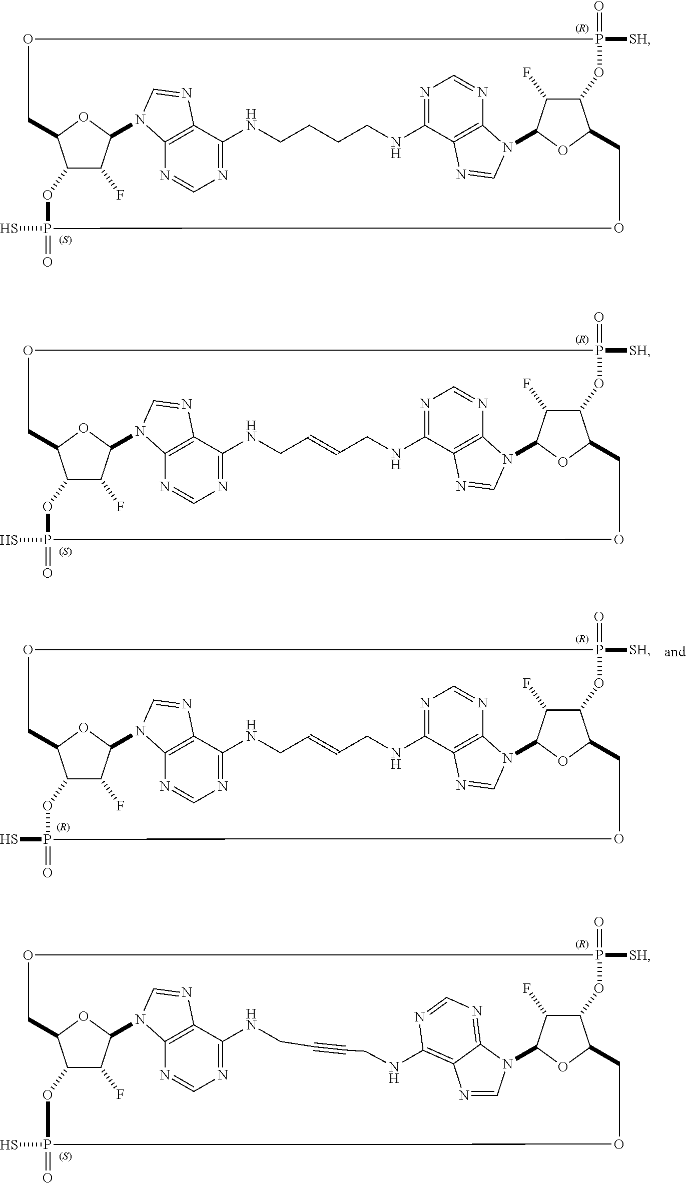

and salts thereof.

In some embodiments, D comprises Compound 1. In some embodiments, D comprises Compound 2.

In some embodiments, L is attached to D via a nitrogen atom at the N-34 nitrogen or the N-39 nitrogen. In some embodiments, L is attached to D at the N-34 nitrogen. In some embodiments, L is attached to D at the N-39 nitrogen.

In some embodiments of linker-payload conjugates comprising L-D, L is any linker disclosed herein. In some embodiments of linker-payload conjugates comprising L-D, D is any drug moiety disclosed herein.

In some embodiments of linker-payload conjugates comprising L-D, wherein L is a cleavable linker that covalently attaches to D, the cleavable linker comprises a cleavable peptide moiety. In some embodiments, the cleavable peptide moiety is cleavable by a protease. In some embodiments, the protease is legumain or cathepsin. In some embodiments, the cleavable peptide moiety comprises an amino acid unit. In some embodiments, the amino acid unit comprises Val-Ala, Val-Cit, Val-Lys, Ala-Ala-Asn, Ala-(NMe)Ala-Asn, Asn, Gly-Gly-Phe-Gly (SEQ ID NO: 244) or Gly-Val-Ala. In some embodiments, the amino acid unit comprises Val-Ala. In some embodiments, the amino acid unit comprises Val-Cit.

In some embodiments, the linker-payload conjugate comprises Val-Ala, and D is selected from a compound disclosed herein. In some embodiments, the linker-payload conjugate comprises Val-Cit, and D is selected from a compound of Table 13.

In some embodiments, the linker-payload conjugate comprises Formula (II), and D is selected from a compound of Table 13.

In some embodiments, the linker-payload conjugate comprises Formula (II)-Val-Ala, and D is selected from a compound of Table 13. In some embodiments, the linker-payload conjugate comprises Formula (II)-Val-Cit, and D is selected from a compound of Table 13.

In some embodiments, the linker-drug conjugate comprises MC-Val-Cit-pABC-Unit 1-Compound 1. In some embodiments, the linker-drug conjugate comprises MC-Val-Ala-pABC-Unit 1-Compound 1 (e.g., LP2). In some embodiments, the linker-drug conjugate comprises MC-Val-Cit-pABC-Unit 2-Compound 1. In some embodiments, the linker-drug conjugate comprises MC-Val-Ala-pABC-Unit 2-Compound 1 (e.g., LP16). In some embodiments, the linker-drug conjugate comprises MC-Val-Cit-pABC-Unit 3-Compound 1. In some embodiments, the linker-drug conjugate comprises MC-Val-Ala-pABC-Unit 3-Compound 1 (e.g., LP20). In some embodiments, the linker-drug conjugate comprises Mal-Formula (II)-Val-Cit-pABC-Unit 2-Compound 1. In some embodiments, the linker-drug conjugate comprises Mal-Formula (II)-Val-Ala-pABC-Unit 2-Compound 1 (e.g., LP54). In some embodiments, the linker-drug conjugate comprises Mal-(PEG)₂-Val-Cit-pABC-Unit 2-Compound 1. In some embodiments, the linker-drug conjugate comprises Mal-(PEG)₂-Val-Ala-pABC-Unit 2-Compound 1 (e.g., LP55).

Exemplary linker-drug conjugates of the invention are disclosed in Table 14, infra. In various embodiments, the linker-drug conjugate is selected from the linker-drug conjugates shown in Table 14.

TABLE 14
Exemplary Linker-Drug Conjugates

| | |
|---|---|
| LP1 | 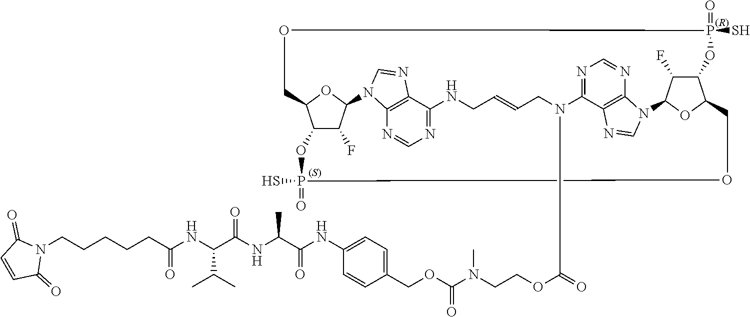 |
| LP2 | 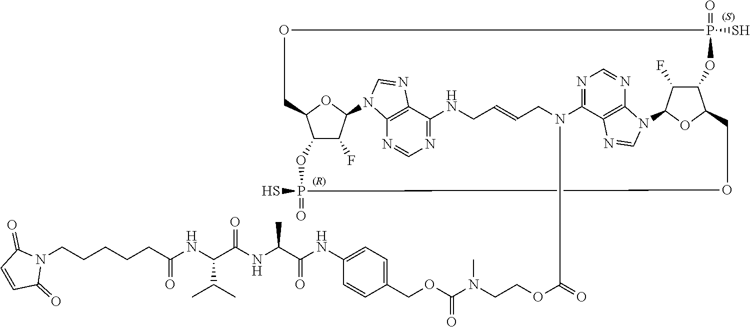 |
| LP3 | 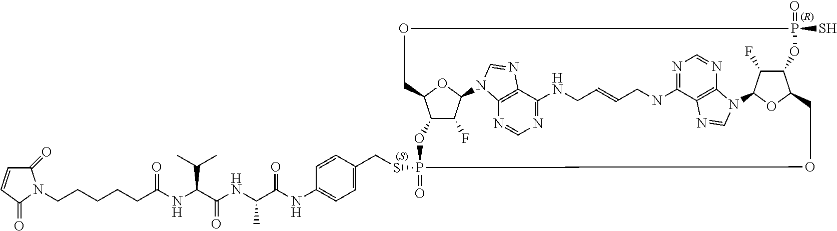 |
| LP4 | 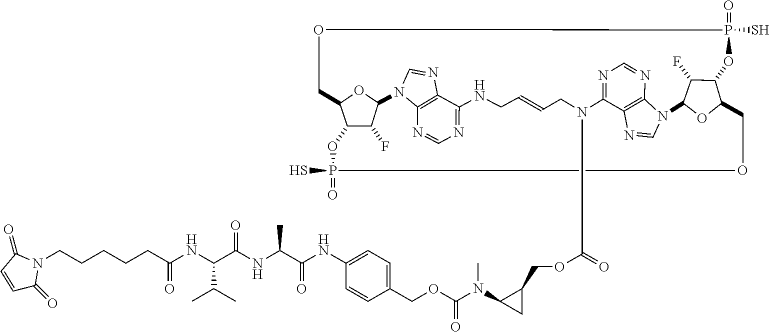 [SMILES: CC(C)[C@H](NC(=O)CCCCCN1C(=O)C=CC1=O)C(=O)N[C@@H](C)C(=O)Nc1ccc(COC(=O)N(C)[C@@H]2C[C@@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1] |
| LP5 | 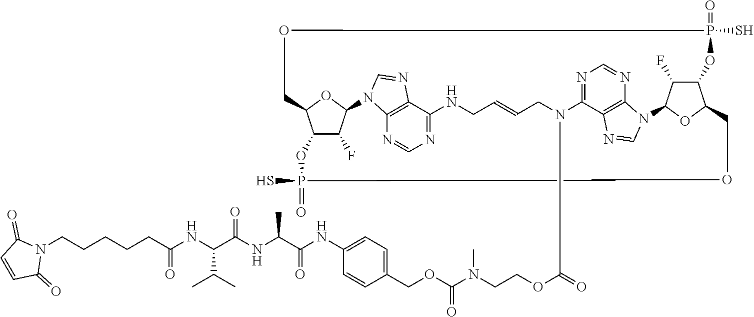 [SMILES: CC(C)[C@H](NC(=O)CCCCCN1C(=O)C=CC1=O)C(=O)N[C@@H](C)C(=O)Nc1ccc(COC(=O)N(C)CCOC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1] |
| LP6 | 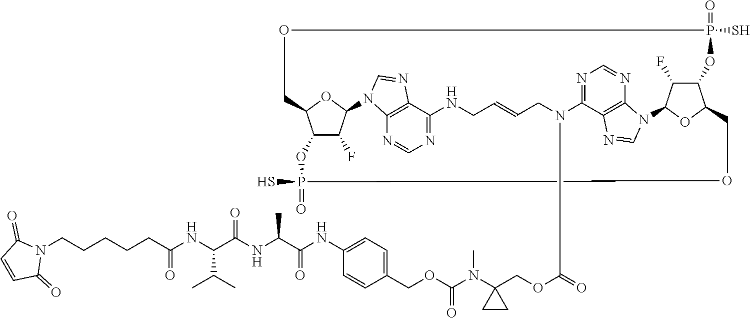 [SMILES: CC(C)[C@H](NC(=O)CCCCCN1C(=O)C=CC1=O)C(=O)N[C@@H](C)C(=O)Nc1ccc(COC(=O)N(C)C2(COC(=O)N3C/C=C/CNc4ncnc5c4ncn5[C@@H]4O[C@@H]5CO[P@](=O)(S)O[C@H]6[C@@H](F)[C@@H](O[C@@H]6CO[P@@](=O)(S)O[C@H]5[C@H]4F)n4cnc5c3ncnc54)CC2)cc1] |
| LP7 | 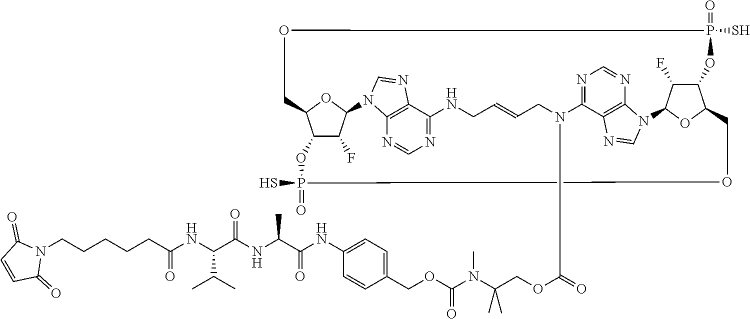 [SMILES: CC(C)[C@H](NC(=O)CCCCCN1C(=O)C=CC1=O)C(=O)N[C@@H](C)C(=O)Nc1ccc(COC(=O)N(C)C(C)(C)COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1] |
| LP8 | 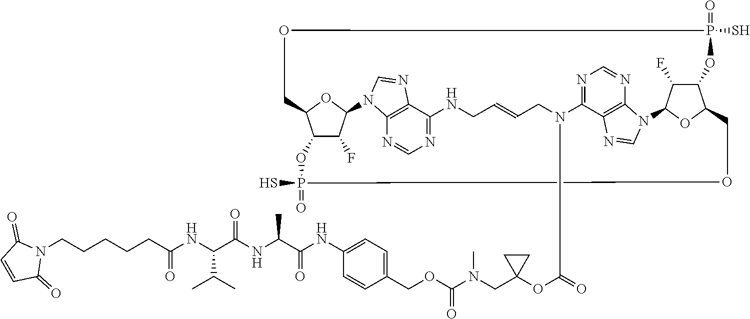 [SMILES: CC(C)[C@H](NC(=O)CCCCCN1C(=O)C=CC1=O)C(=O)N[C@@H](C)C(=O)Nc1ccc(COC(=O)N(C)CC2(OC(=O)N3C/C=C/CNc4ncnc5c4ncn5[C@@H]4O[C@@H]5CO[P@](=O)(S)O[C@H]6[C@@H](F)[C@@H](O[C@@H]6CO[P@@](=O)(S)O[C@H]5[C@H]4F)n4cnc5c3ncnc54)CC2)cc1] |
| LP9 | 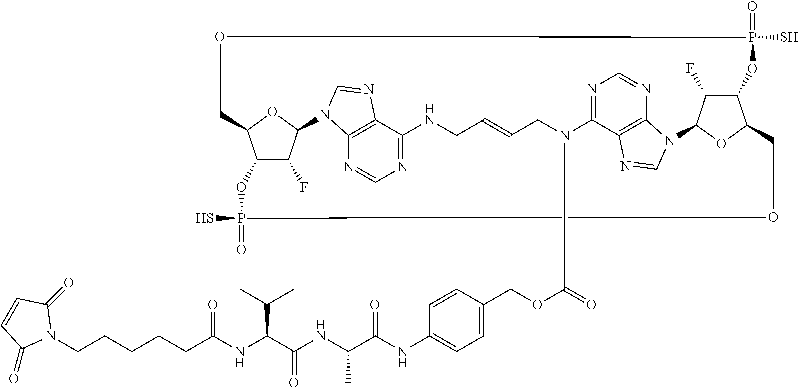 [SMILES: CC(C)[C@H](NC(=O)CCCCCN1C(=O)C=CC1=O)C(=O)N[C@@H](C)C(=O)Nc1ccc(COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1] |
| LP10 | 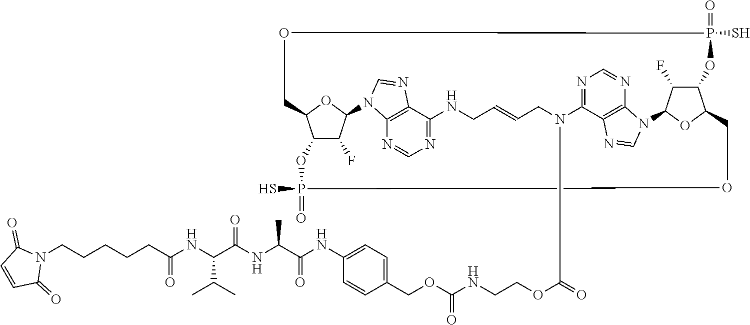 [SMILES: CC(C)[C@H](NC(=O)CCCCCN1C(=O)C=CC1=O)C(=O)N[C@@H](C)C(=O)Nc1ccc(COC(=O)NCCOC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1] |
| LP11 | 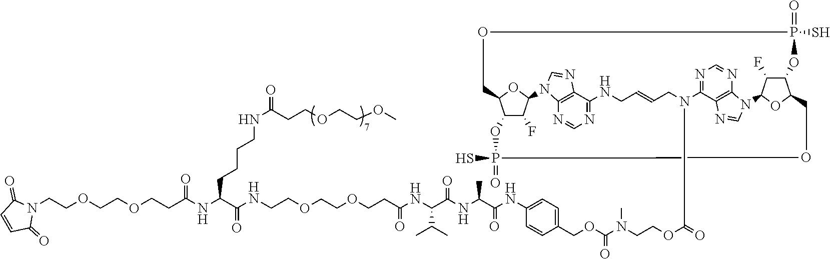 |
| LP12 | 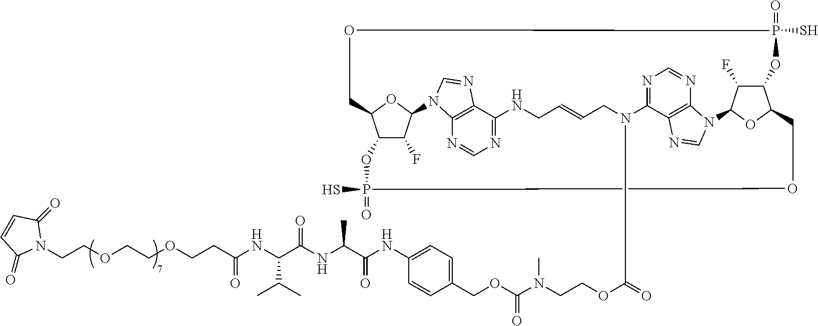 |
| LP13 | 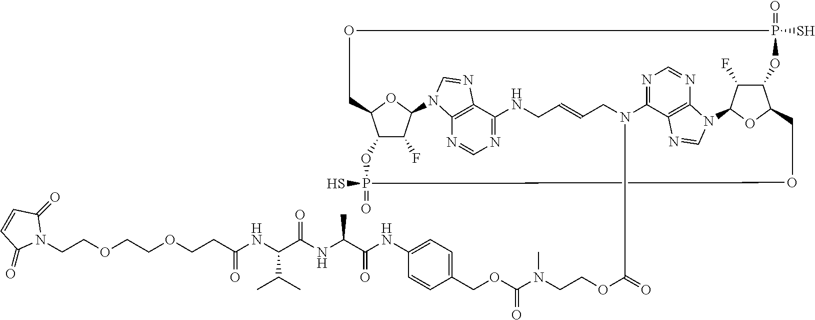 [SMILES: CC(C)[C@H](NC(=O)CCOCCOCCN1C(=O)C=CC1=O)C(=O)N[C@@H](C)C(=O)Nc1ccc(COC(=O)N(C)CCOC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1] |
| LP14 | 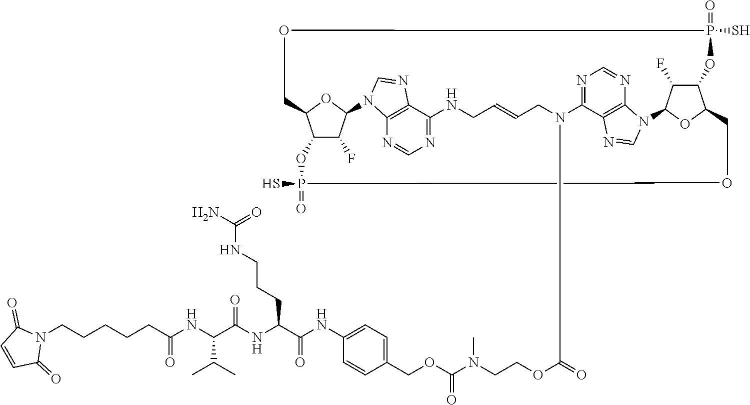 [SMILES: CC(C)[C@H](NC(=O)CCCCCN1C(=O)C=CC1=O)C(=O)N[C@@H](CCCNC(N)=O)C(=O)Nc1ccc(COC(=O)N(C)CCOC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1] |
| LP15 | 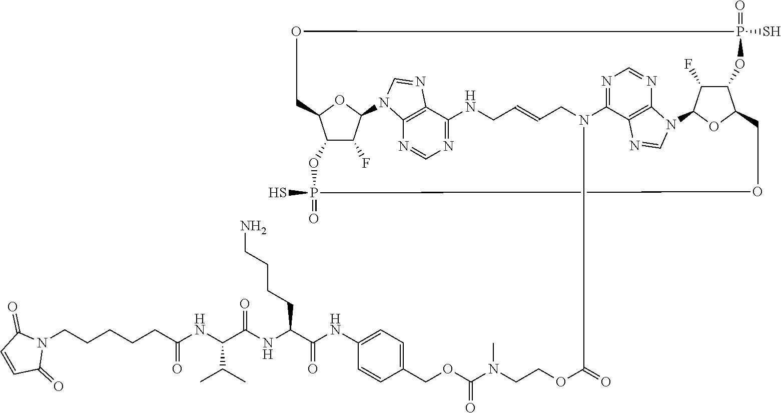 [SMILES: CC(C)[C@H](NC(=O)CCCCCN1C(=O)C=CC1=O)C(=O)N[C@@H](CCCCN)C(=O)Nc1ccc(COC(=O)N(C)CCOC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1] |
| LP16 | 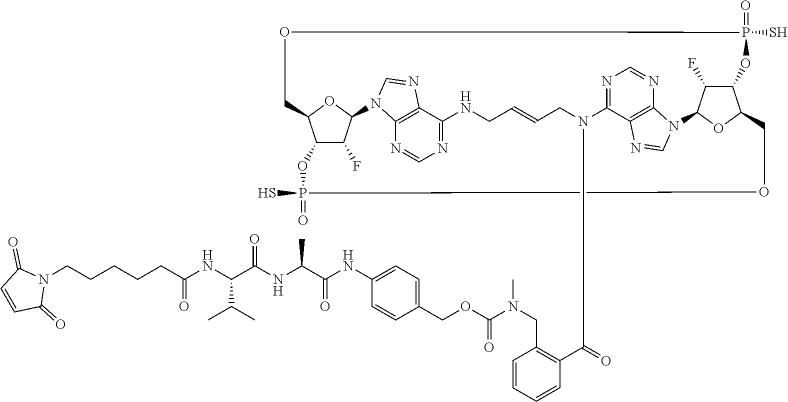 [SMILES: CC(C)[C@H](NC(=O)CCCCCN1C(=O)C=CC1=O)C(=O)N[C@@H](C)C(=O)Nc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1] |
| LP17 | 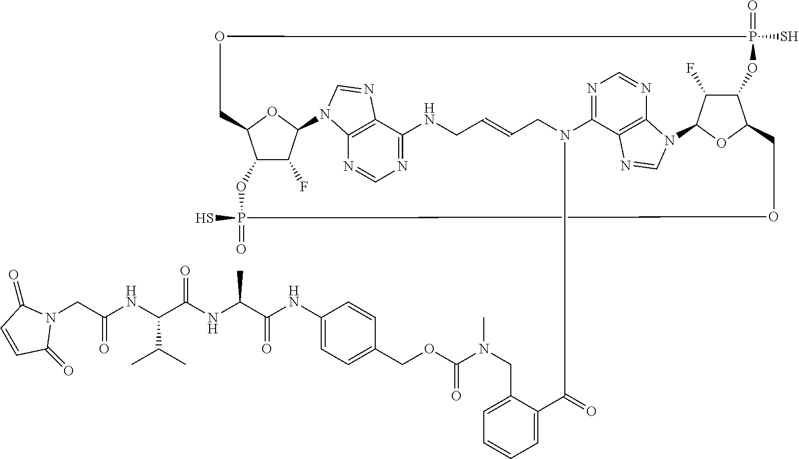 [SMILES: CC(C)[C@H](NC(=O)CN1C(=O)C=CC1=O)C(=O)N[C@@H](C)C(=O)Nc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1] |
| LP18 | 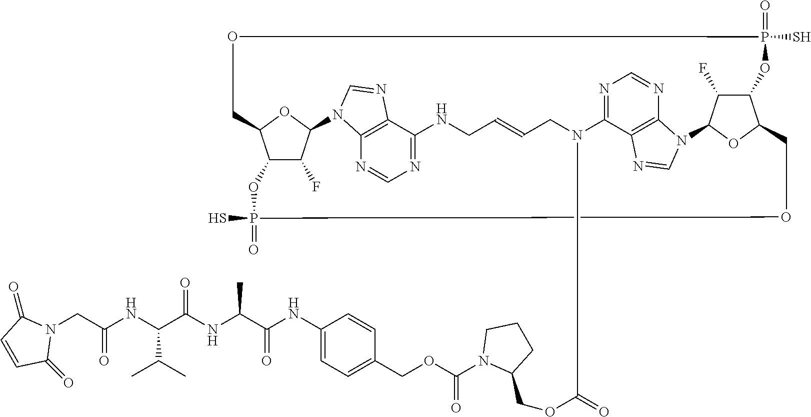 [SMILES: CC(C)[C@H](NC(=O)CN1C(=O)C=CC1=O)C(=O)N[C@@H](C)C(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1] |
| LP19 | 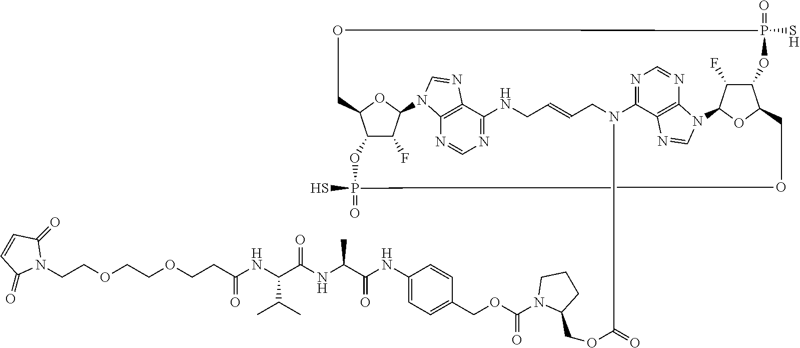 [SMILES: CC(C)[C@H](NC(=O)CCOCCOCCN1C(=O)C=CC1=O)C(=O)N[C@@H](C)C(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1] |
| LP20 | 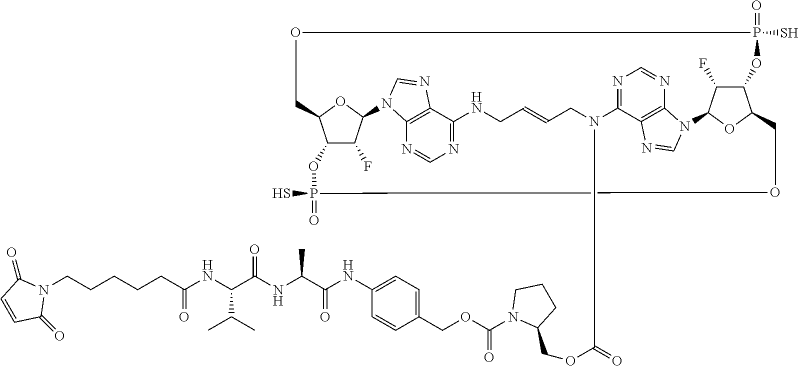 |
| LP21 | 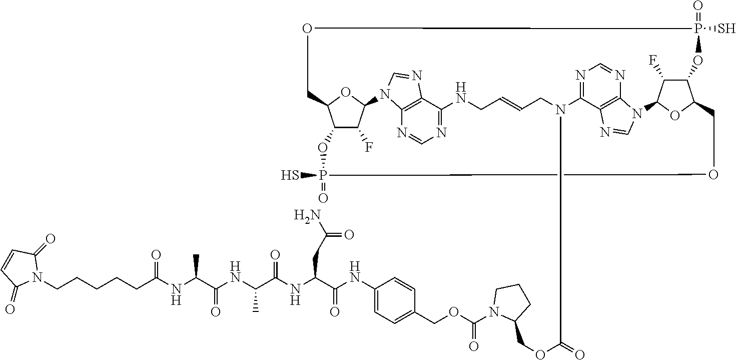 [SMILES: C[C@H](NC(=O)CCCCCN1C(=O)C=CC1=O)C(=O)N[C@@H](C)C(=O)N[C@@H](CC(N)=O)C(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1] |
| LP22 | 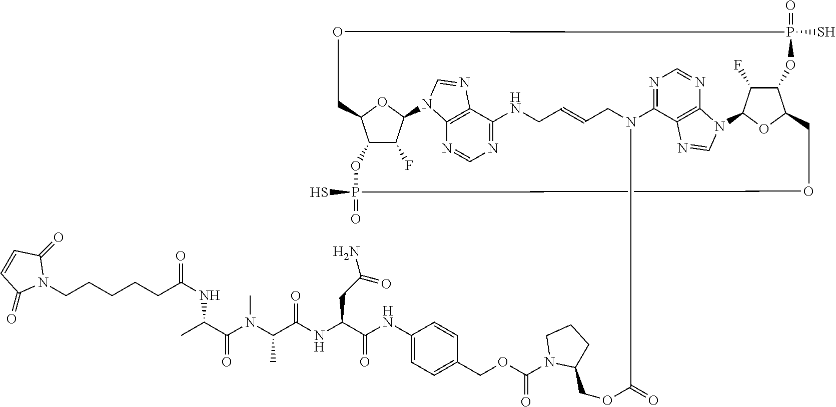 |
| LP23 | 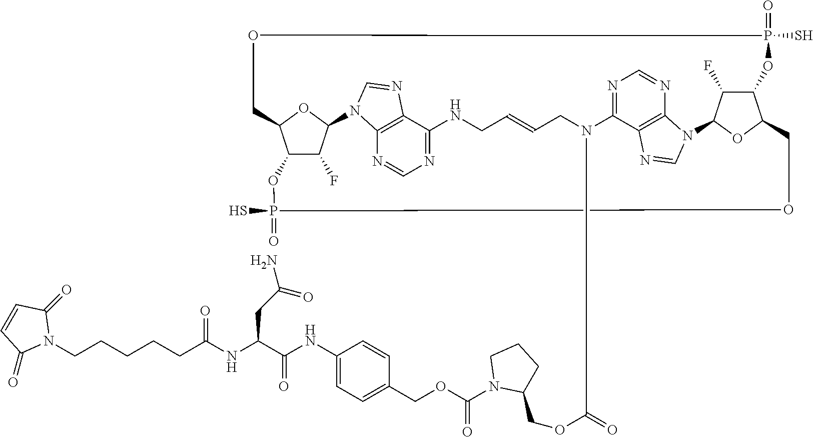 [SMILES: NC(=O)C[C@H](NC(=O)CCCCCN1C(=O)C=CC1=O)C(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1] |
| LP24 (SEQ ID NO: 247) | 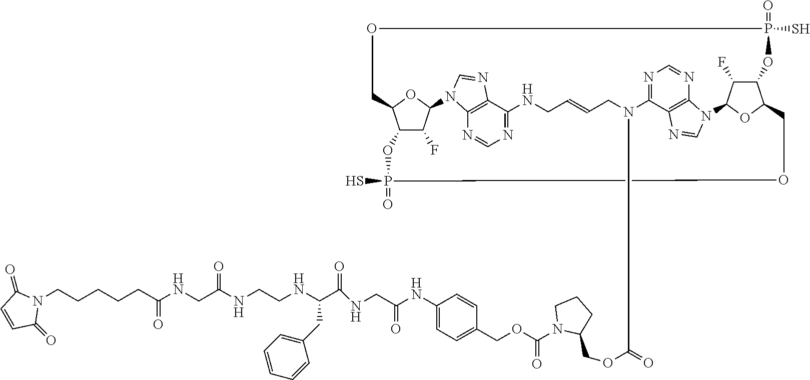 [SMILES: O=C(CCCCCN1C(=O)C=CC1=O)NCC(=O)NCCN[C@@H](Cc1ccccc1)C(=O)NCC(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1] |
| LP25 | 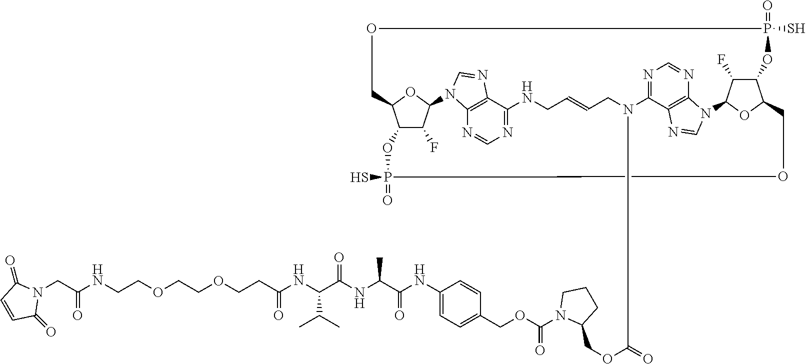 [SMILES: CC(C)[C@H](NC(=O)CCOCCOCCNC(=O)CN1C(=O)C=CC1=O)C(=O)N[C@@H](C)C(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1] |
| LP26 | 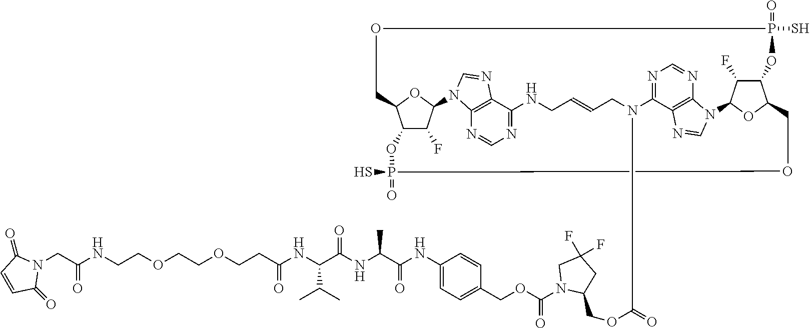 |
| LP27 | 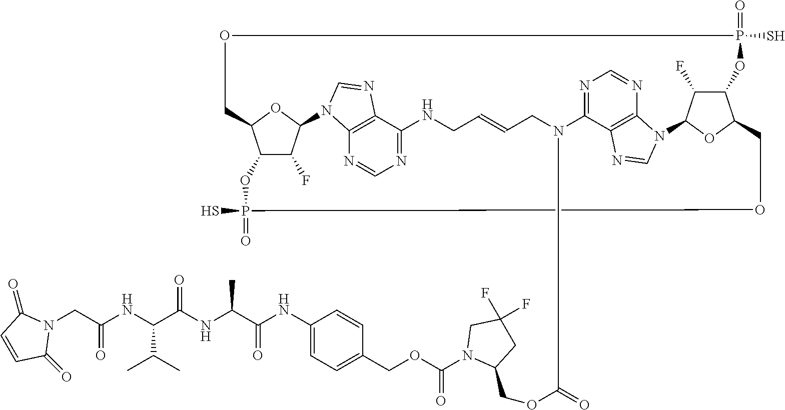 [SMILES: CC(C)[C@H](NC(=O)CN1C(=O)C=CC1=O)C(=O)N[C@@H](C)C(=O)Nc1ccc(COC(=O)N2CC(F)(F)C[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1] |
| LP28 | 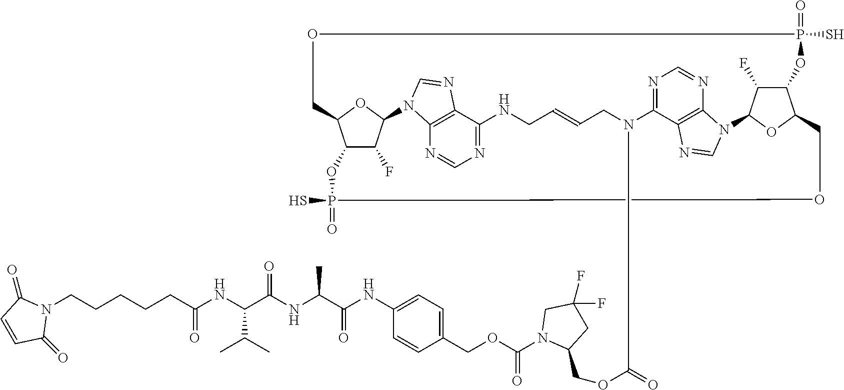 [SMILES: CC(C)[C@H](NC(=O)CCCCCN1C(=O)C=CC1=O)C(=O)N[C@@H](C)C(=O)Nc1ccc(COC(=O)N2CC(F)(F)C[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1] |
| LP29 | 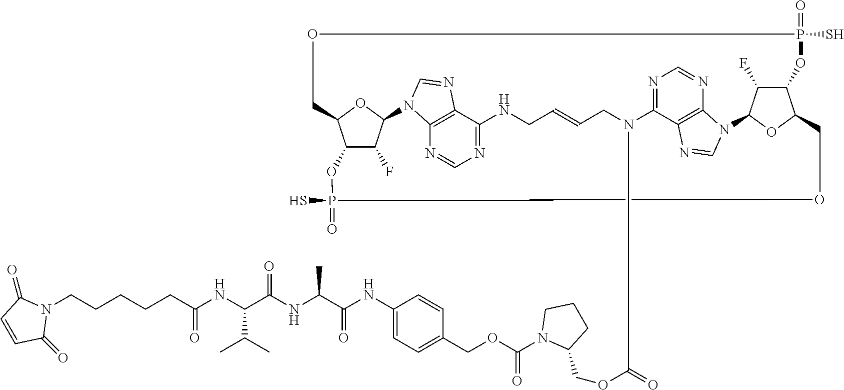 |
| LP30 | [symbol] |
| LP31 | [symbol] |
| LP32 | 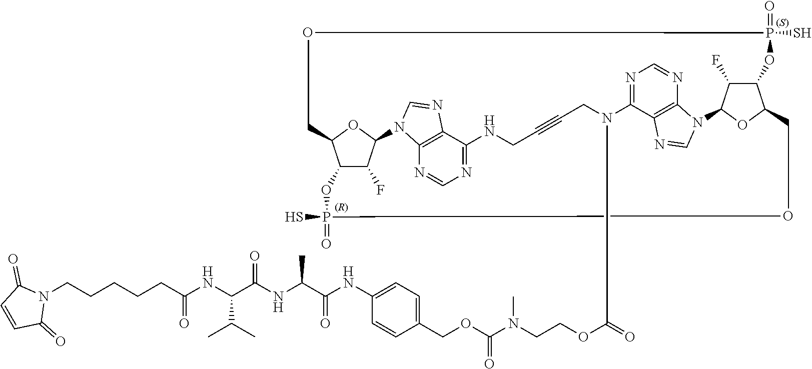 |
| LP54 | [symbol] |
| LP55 | 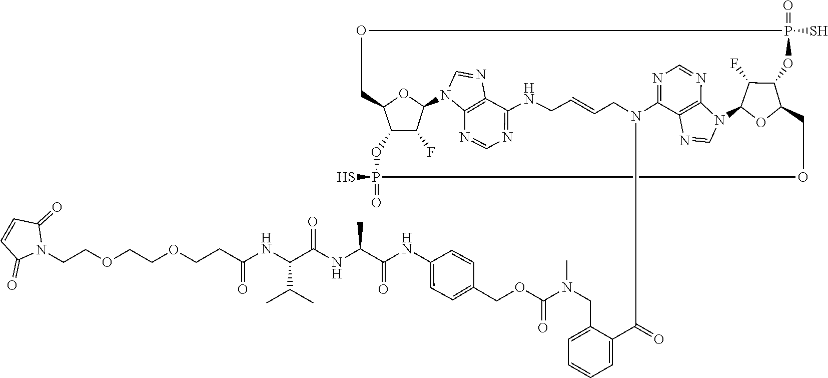 [SMILES: CC(C)[C@H](NC(=O)CCOCCOCCN1C(=O)C=CC1=O)C(=O)N[C@@H](C)C(=O)Nc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1] |

[symbol]

indicates data missing or illegible when filed

In some embodiments, an exemplary linker-drug conjugate or a salt thereof may be referred to as “LP2” and has the structure of LP2 shown below:

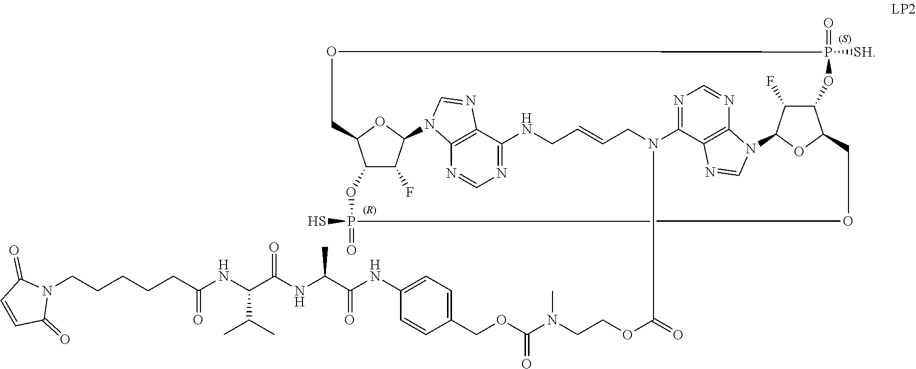

In some embodiments, an exemplary linker-drug conjugate or a salt thereof may be referred to as “LP16” and has the structure of LP16 shown below:

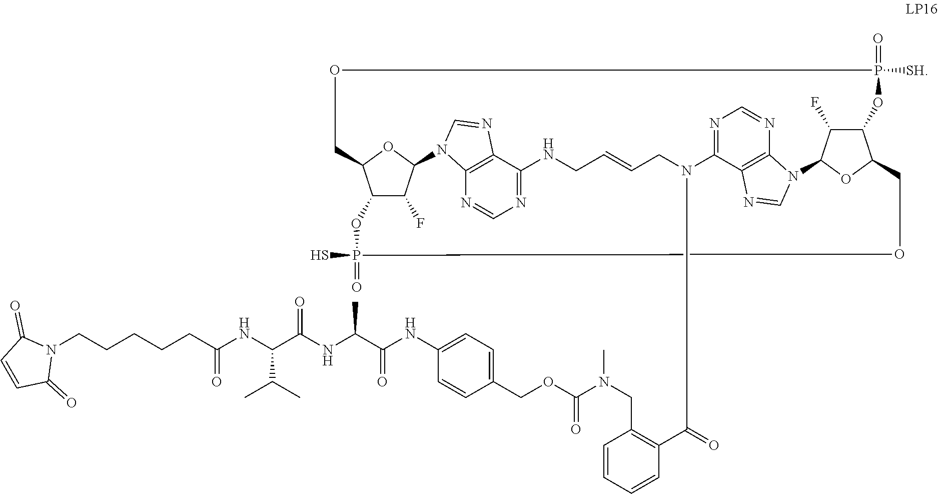 [SMILES: CC(C)[C@H](NC(=O)CCCCCN1C(=O)C=CC1=O)C(=O)N[C@@H](C)C(=O)Nc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

In some embodiments, an exemplary linker-drug conjugate or a salt thereof may be referred to as “LP20” and has the structure of LP20 shown below:

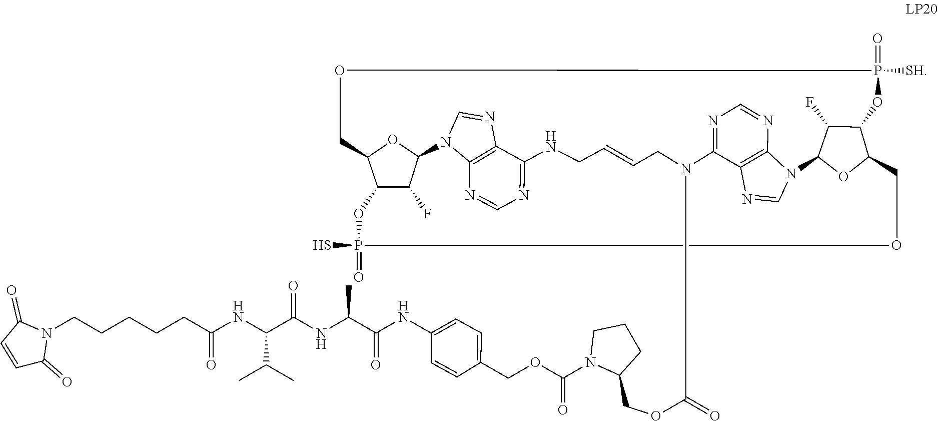 [SMILES: CC(C)[C@H](NC(=O)CCCCCN1C(=O)C=CC1=O)C(=O)N[C@@H](C)C(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

In some embodiments, an exemplary linker-drug conjugate or a salt thereof may be referred to as “LP54” and has the structure of LP54 shown below:

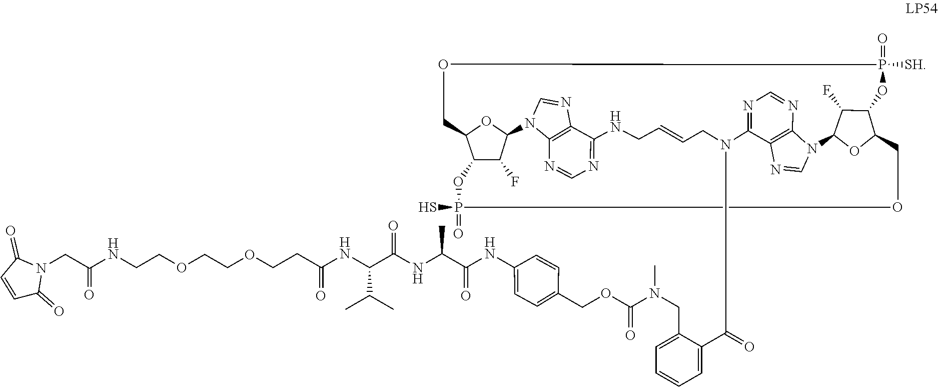 [SMILES: CC(C)[C@H](NC(=O)CCOCCOCCNC(=O)CN1C(=O)C=CC1=O)C(=O)N[C@@H](C)C(=O)Nc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

In some embodiments, an exemplary linker-drug conjugate or a salt thereof may be referred to as “LP55” and has the structure of LP55 shown below:

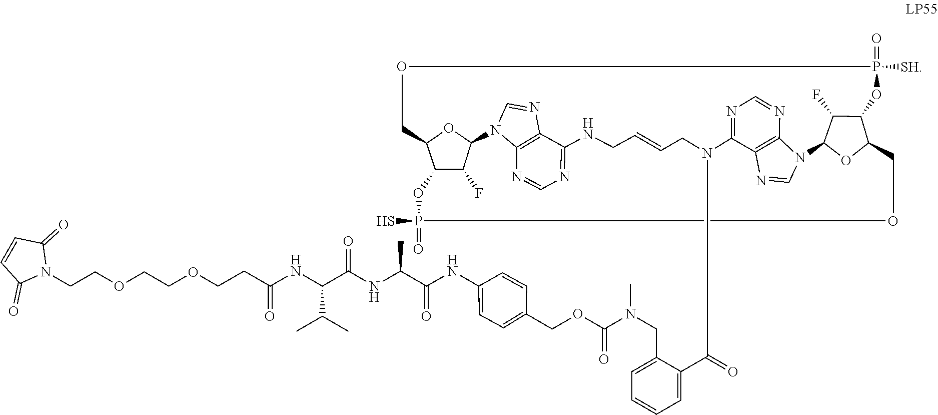 [SMILES: CC(C)[C@H](NC(=O)CCOCCOCCN1C(=O)C=CC1=O)C(=O)N[C@@H](C)C(=O)Nc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

In some embodiments, a linker-payload disclosed herein, e.g., LP2, LP16, LP20, LP54, or LP55, has improved properties over prior linker-STING agonist conjugates. In some embodiments, a linker-payload disclosed herein, e.g., LP2, LP16, LP20, LP54, or LP55, has superior plasma stability over prior art linker-STING agonist conjugates. In some embodiments, a linker-payload disclosed herein, e.g., LP2, LP16, LP20, LP54, or LP55, has superior in vivo anti-tumor activity over prior art linker-STING agonist conjugates. In some embodiments, a linker-payload disclosed herein, e.g., LP2, LP16, LP20, LP54, or LP55, has superior tolerability in vivo over prior art linker-STING agonist conjugates.

In some embodiments of linker-payload conjugates disclosed herein, wherein D is a STING agonist, e.g., a compound of Table 13, e.g., Compound 1, and L is conjugated to D at the N-34 nitrogen or the N-39 nitrogen (e.g., LP2, LP16, LP20, LP54, or LP55), the linker-payload conjugate demonstrates superior properties (e.g., plasma stability, in vitro immune responses, in vivo anti-tumor activity, tolerability, stimulation of an anti-tumor immune response in the tumor microenvironment) compared to other linker-payload conjugates comprising a compound of Table 13 that are conjugated to D at alternative attachment points, e.g., at a sulfur, e.g., S-2 or S-14. Exemplary evidence of the superior benefits of such linker-payload conjugates are shown in Examples 4, 5, and 7.

In some embodiments, an ADC disclosed herein comprises a cleavable linker and an internalizing anti-CD73 antibody or antigen-binding fragment thereof as described herein. In some embodiments, the anti-CD73 antibody or antigen-binding fragment thereof comprises three HCDRs comprising amino acid sequences of SEQ ID NO: 1 (HCDR1), SEQ ID NO: 2 (HCDR2), and SEQ ID NO: 3 (HCDR3); and three LCDRs comprising SEQ ID NO: 4 (LCDR1), SEQ ID NO: 5 (LCDR2), and SEQ ID NO: 6 (LCDR3), as defined by the Kabat numbering system. In some embodiments, the anti-CD73 antibody or antigen-binding fragment thereof comprises three HCDRs comprising amino acid sequences of SEQ ID NO: 7 (HCDR1), SEQ ID NO: 8 (HCDR2), and SEQ ID NO: 9 (HCDR3); and three LCDRs comprising SEQ ID NO: 10 (LCDR1), SEQ ID NO: 11 (LCDR2), and SEQ ID NO: 12 (LCDR3), as defined by the IMGT numbering system.

In some embodiments, the anti-CD73 antibody or antigen-binding fragment thereof comprises a heavy chain variable region comprising an amino acid sequence of SEQ ID NO: 49, and a light chain variable region comprising an amino acid sequence of SEQ ID NO: 50. In some embodiments, the anti-CD73 antibody or antigen-binding fragment thereof comprises a heavy chain variable region comprising an amino acid sequence of SEQ ID NO: 49, and a light chain variable region comprising an amino acid sequence of SEQ ID NO: 51.

In some embodiments, an ADC disclosed herein comprises a cleavable linker and an internalizing anti-CD73 antibody or antigen-binding fragment thereof as described herein. In some embodiments, the anti-CD73 antibody or antigen-binding fragment thereof comprises three HCDRs comprising amino acid sequences of SEQ ID NO: 13 (HCDR1), SEQ ID NO: 14 (HCDR2), and SEQ ID NO: 15 (HCDR3); and three LCDRs comprising SEQ ID NO: 16 (LCDR1), SEQ ID NO: 17 (LCDR2), and SEQ ID NO: 18 (LCDR3), as defined by the Kabat numbering system. In some embodiments, the anti-CD73 antibody or antigen-binding fragment thereof comprises three HCDRs comprising amino acid sequences of SEQ ID NO: 19 (HCDR1), SEQ ID NO: 20 (HCDR2), and SEQ ID NO: 21 (HCDR3); and three LCDRs comprising SEQ ID NO: 22 (LCDR1), SEQ ID NO: 23 (LCDR2), and SEQ ID NO: 24 (LCDR3), as defined by the IMGT numbering system.

In some embodiments, the anti-CD73 antibody or antigen-binding fragment thereof comprises a heavy chain variable region comprising an amino acid sequence of SEQ ID NO: 52, and a light chain variable region comprising an amino acid sequence of SEQ ID NO: 53. In some embodiments, the anti-CD73 antibody or antigen-binding fragment thereof comprises a heavy chain variable region comprising an amino acid sequence of SEQ ID NO: 52, and a light chain variable region comprising an amino acid sequence of SEQ ID NO: 54.

In some embodiments, p is from 1 to 12, or 2 to 11. In some embodiments, p is from 1 to 8. In some embodiments, p is from 4 to 11. In some embodiments, p is from 4 to 8. In some embodiments, p is 2. In some embodiments, p is 4. In some embodiments, p is 7. In some embodiments, p is 11.

### Antibody-Drug Conjugates

In various embodiments, an anti-CD73 antibody moiety or an antigen-binding fragment thereof as disclosed herein may be conjugated (i.e., covalently attached, e.g., by a linker) to one or more drug moieties, wherein the one or more drug moieties when not conjugated to an antibody moiety have a cytotoxic or cytostatic effect. In some embodiments, the one or more drug moieties exhibit reduced or no cytotoxicity when bound in a conjugate but resume cytotoxicity after cleavage from the linker and antibody moiety.

The development and production of an ADC for use as a human therapeutic agent, e.g., as an oncologic agent, may require more than the identification of an antibody capable of binding to a desired target or targets and attaching to a drug used on its own to treat cancer. Linking the antibody to the drug may have significant and unpredictable effects on the activity of one or both of the antibody and the drug, effects which will vary depending on the type of linker and/or drug chosen. In some embodiments, therefore, the components of the ADC are selected to (i) retain one or more therapeutic properties exhibited by the antibody and drug moieties in isolation, (ii) maintain the specific binding properties of the antibody moiety; (iii) optimize drug loading and drug-to-antibody ratios; (iv) allow targeted tumor cell delivery, e.g., intracellular delivery, of the drug moiety via stable attachment to the antibody moiety; (v) reduce toxicity compared to non-targeted and/or systemic delivery of the drug moiety; (vi) retain ADC stability as an intact conjugate until transport or delivery to a target site; (vii) minimize aggregation of the ADC prior to or after administration; (viii) exhibit in vivo anti-cancer treatment efficacy comparable to or superior to that of the antibody and drug moieties in isolation; (ix) minimize off-target killing by the drug moiety; (x) exhibit desirable pharmacokinetic and pharmacodynamics properties, formulatability, and toxicologic/immunologic profiles; (xi) maintain stimulation of an anti-tumor immune response in the tumor microenvironment; and/or (xii) increase phagocytosis of CD73-expressing cells by myeloid cells (e.g., macrophages or dendritic cells). Screening each of these properties may be needed to identify an improved ADC for therapeutic use. See, e.g., Ab et al. (2015) Mol. Cancer Ther. 14:1605-13.

The ADC compounds of the present disclosure may selectively deliver an effective dose of a cytotoxic or cytostatic agent to cancer cells or to tumor tissue. It has been discovered that the disclosed ADCs have potent cytotoxic and/or cytostatic activity against cells expressing CD73. In some embodiments, the cytotoxic and/or cytostatic activity of the ADC is dependent on CD73 expression level in a cell and/or on a cell surface. In some embodiments, the disclosed ADCs are particularly effective at killing cancer cells expressing a high level of CD73, as compared to cancer cells expressing the same antigen at a low level. Exemplary high CD73-expressing cancers include but are not limited to acute myeloid leukemia, breast cancer, cervical cancer, colorectal cancer, diffuse large B-cell lymphoma, esophageal cancer, gallbladder cancer, gastric cancer, glioblastoma, head and neck cancer, head and neck squamous carcinoma, melanoma, non-small-cell lung cancer (NSCLC), ovarian cancer, pancreatic cancer, pancreatic ductal adenocarcinoma (PDAC), rectum adenocarcinoma, renal cell carcinoma, sarcoma, stomach adenocarcinoma, T-cell lymphoma, or thyroid carcinoma. In some embodiments, the disclosed ADCs are particularly effective at killing high CD73-expressing cancers such as breast cancer, ovarian cancer, PDAC, or NSCLC. In some embodiments, targeted killing of CD73-expressing cancer cells is improved by the presence or recruitment of myeloid cells (e.g., macrophages and/or dendritic cells).

In some embodiments, the disclosed ADCs have substitutions in residues in the Fc domain of the anti-CD73 antibody relative to a wild-type Fc domain. In some embodiments, the disclosed ADCs comprise an anti-CD73 antibody that comprises an IgG1 Fc domain that has been mutated to reduce binding to a Fcγ receptor (FcγR) as compared to an IgG1 Fc-containing antibody with a wild type IgG1 Fc domain. These substitutions may reduce the ability of the ADC to bind to various cell receptors, such as a Fcγ receptor (FcγR), and other immune molecules, as compared to an ADC comprising an anti-CD73 antibody without these substitutions.

In some embodiments, the disclosed ADCs demonstrate superior properties (e.g., antigen-binding specificity (e.g., epitope and/or affinity), site-specific linker-payload conjugation, tolerability, in vitro cytotoxicity, safety profile) compared to other CD73 ADCs. In some embodiments, the disclosed ADCs have superior antigen-binding specificity compared to other CD73 ADCs. In some embodiments, the disclosed ADCs have superior site-specific linker-payload conjugation compared to other CD73 ADCs. In some embodiments, the disclosed ADCs have superior tolerability compared to other CD73 ADCs (e.g., 79G9). In some embodiments, the disclosed ADCs have superior in vitro cytotoxicity compared to other CD73 ADCs. In some embodiments, the disclosed ADCs have a superior safety profile (e.g., reduced Fey receptor interactions) compared to other CD73 ADCs. In some embodiments, the disclosed ADCs have a superior property in more than one of the listed categories (e.g., all of them).

In some embodiments, ADCs disclosed herein demonstrate CD73-specific binding on CD73-expressing cells, e.g., in CD73-expressing cancers. In some embodiments, upon binding CD73, the disclosed ADCs are internalized. In some embodiments, release of the drug moiety, e.g., Compound 1, results in STING pathway activation and release of proinflammatory cytokines (e.g., IFNβ). In some embodiments, release of proinflammatory cytokines promotes myeloid cell activation. In some embodiments, release of proinflammatory cytokines stimulates Type I IFN-dependent anti-tumor activity.

In some embodiments, the disclosed ADCs activate myeloid cells, e.g., macrophages or dendritic cells. Without being bound by theory, myeloid cell activation may be a result of phagocytosis of CD73-expressing cancer cells bound by the disclosed ADCs. In some embodiments, the disclosed ADCs activate macrophages. in some embodiments, the activated macrophages are proinflammatory (M1) macrophages. In some embodiments, tumor-associated macrophages or M2 macrophages undergo proinflammatory activation upon administration of the disclosed ADCs. In some embodiments, the activated macrophages release pro-inflammatory cytokines and chemokines (e.g., TNFα, CXCL10, IL-6, IFNβ, and/or IL-1β). In some embodiments, the activated macrophages promote further myeloid cell activation. In some embodiments, the activated macrophages promote the generation of cytotoxic T cells. In some embodiments, the activated macrophages demonstrate increased phagocytosis of cancer cells. In some embodiments, administration of the disclosed ADCs stimulates IFN-dependent anti-tumor activity. As used herein, an “activated macrophage” is synonymous with a “polarized macrophage.”

Provided herein are ADC compounds comprising an antibody or antigen-binding fragment thereof (Ab) which targets a tumor cell, a drug moiety (D), and a linker moiety (L) that covalently attaches Ab to D. In certain aspects, the antibody or antigen-binding fragment is able to bind to a tumor-associated antigen (e.g., CD73) with high specificity and high affinity. In certain embodiments, the antibody or antigen-binding fragment is internalized into a target cell upon binding, e.g., into a degradative compartment in the cell. In some embodiments, ADCs internalize upon binding to a target cell, undergo degradation, and release the drug moiety. The drug moiety may be released from the antibody and/or the linker moiety of the ADC by enzymatic action, hydrolysis, oxidation, or any other mechanism.

In some embodiments, target cells bound by the ADC are phagocytosed by a myeloid cell, e.g., a macrophage or dendritic cell. In some embodiments, upon phagocytosis the ADCs undergo degradation and release the drug moiety. In some embodiments, the drug moiety is released in the phagolysosome of the myeloid cell (e.g., a macrophage or dendritic cell). The drug moiety may be released from the antibody and/or the linker moiety of the ADC by enzymatic action, hydrolysis, oxidation, or any other mechanism.

An exemplary ADC has Formula I:

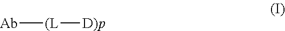

wherein Ab=antibody moiety (i.e., antibody or antigen-binding fragment), L=linker moiety, D=drug moiety, and p=the number of drug moieties per antibody moiety.

In some embodiments, an antibody-drug conjugate disclosed herein comprises an anti-CD73 antibody or antigen-binding fragment. In some embodiments, the anti-CD73 antibody or antigen-binding fragment comprises a heavy chain having an amino acid sequence of SEQ ID NOs: 75-78, 81-84, 87-90, and 93-96 listed in Table 8, supra and/or comprising a set of CDRs and/or a variable domain from the amino acid sequences in Table 8. In some embodiments, the anti-CD73 antibody or antigen-binding fragment comprises a light chain having an amino acid sequence of SEQ ID NOs: 79-80, 85-86, 91-92, and 97-98 listed in Table 8, supra and/or comprising a set of CDRs and/or a variable domain from the amino acid sequences in Table 8.

In some embodiments, the anti-CD73 antibody or antigen-binding fragment comprises three heavy chain CDRs and three light chain CDRs as follows: HCDR1 comprising SEQ ID NO: 1, HCDR2 comprising SEQ ID NO: 2, HCDR3 comprising SEQ ID NO: 3; LCDR1 comprising SEQ ID NO: 4, LCDR2 comprising SEQ ID NO: 5, and LCDR3 comprising SEQ ID NO: 6, as defined by the Kabat numbering system. In some embodiments, an anti-CD73 antibody or antigen-binding fragment thereof provided herein comprises three heavy chain CDRs and three light chain CDRs as follows: HCDR1 comprising SEQ ID NO: 7, HCDR2 comprising SEQ ID NO: 8, HCDR3 comprising SEQ ID NO: 9; LCDR1 comprising SEQ ID NO: 10, LCDR2 comprising SEQ ID NO: 11, and LCDR3 comprising SEQ ID NO: 12, as defined by the IMGT numbering system.

In some embodiments, the anti-CD73 antibody or antigen-binding fragment comprises a heavy chain variable region comprising an amino acid sequence of SEQ ID NO: 49, and a light chain variable region comprising an amino acid sequence of SEQ ID NO: 50. In some embodiments, the anti-CD73 antibody or antigen-binding fragment comprises a heavy chain variable region comprising an amino acid sequence of SEQ ID NO: 49, and a light chain variable region comprising an amino acid sequence of SEQ ID NO: 51.

In some embodiments, an anti-CD73 antibody or antigen-binding fragment comprises three heavy chain CDRs and three light chain CDRs as follows: HCDR1 comprising SEQ ID NO: 13, HCDR2 comprising SEQ ID NO: 14, HCDR3 comprising SEQ ID NO: 15; LCDR1 comprising SEQ ID NO: 16, LCDR2 comprising SEQ ID NO: 17, and LCDR3 comprising SEQ ID NO: 18, as defined by the Kabat numbering system. In some embodiments, an anti-CD73 antibody or antigen-binding fragment comprises three heavy chain CDRs and three light chain CDRs as follows: HCDR1 comprising SEQ ID NO: 19, HCDR2 comprising SEQ ID NO: 20, HCDR3 comprising SEQ ID NO: 21; LCDR1 comprising SEQ ID NO: 22, LCDR2 comprising SEQ ID NO: 23, and LCDR3 comprising SEQ ID NO: 24, as defined by the IMGT numbering system.

In some embodiments, the anti-CD73 antibody or antigen-binding fragment comprises a heavy chain variable region comprising an amino acid sequence of SEQ ID NO: 52, and a light chain variable region comprising an amino acid sequence of SEQ ID NO: 53. In some embodiments, the anti-CD73 antibody or antigen-binding fragment comprises a heavy chain variable region comprising an amino acid sequence of SEQ ID NO: 52, and a light chain variable region comprising an amino acid sequence of SEQ ID NO: 54.

To accomplish site-specific conjugation of linkers and/or drug moieties to an antibody or antigen-binding fragment thereof, in some embodiments, a linker comprising a thiol-reactive group is used to generate a conjugated antibody or antigen-binding fragment, e.g., by reacting with the antibody or antigen-binding fragment at a cysteine residue. In some embodiments, the cysteine residue is at amino acid position 80 on the light chain. In some embodiments, the cysteine residue is at amino acid position 118 on the heavy chain. Methods to accomplish site-specific conjugation of linkers and/or drug moieties to an antibody or antigen-binding fragment for the production of ADCs are known in the art and disclosed in PCT application WO 2016/205618, herein incorporated by reference in its entirety.

### Drug Loading

Drug loading is represented by p and is also referred to herein as the drug-to-antibody ratio (DAR). In some embodiments, drug loading may range from 1 to 20 (i.e., 1 to 20 copies of the linker-drug attached to each antibody moiety), e.g., 1 to 12 drug moieties per antibody moiety. In some embodiments, p is an integer from 1 to 12. In some embodiments, p is an integer from 1 to 8. In some embodiments, p is an integer from 1 to 12, 1 to 11, 1 to 10, 1 to 9, 1 to 8, 1 to 7, 1 to 6, 1 to 5, 1 to 4, 1 to 3, or 1 to 2. In some embodiments, p is an integer from 2 to 12, 2 to 11, 2 to 10, 2 to 9, 2 to 8, 2 to 7, 2 to 6, 2 to 5, 2 to 4, or 2 to 3. In some embodiments, p is an integer from 2 to 11. In some embodiments, p is 1, 2, 3, 4, 5, 6, 7, 8, 9, 10, 11, or 12. In some embodiments, p is 2. In some embodiments, p is 4. In some embodiments, p is 7. In some embodiments, p is 8. In some embodiments, p is 11. In some embodiments, drug loading may be expressed as an average loading in a population of antibodies, e.g., an average loading of about 1-12, e.g., about 2-11. In some embodiments, the average drug loading in a population of antibodies is about 2 to about 8. In some embodiments, the average drug loading in a population of antibodies is about 2, about 4, or about 8.

Drug loading may be limited by the number of attachment sites on the antibody moiety. In some embodiments, the linker moiety (L) of the ADC attaches to the antibody moiety through a chemically active group on one or more amino acid residues on the antibody moiety. For example, the linker may be attached to the antibody moiety via a free amino, imino, hydroxyl, thiol, or carboxyl group (e.g., to the N- or C-terminus, to the epsilon amino group of one or more lysine residues, to the free carboxylic acid group of one or more glutamic acid or aspartic acid residues, or to the sulfhydryl group of one or more cysteine residues). The site to which the linker is attached can be a natural residue in the amino acid sequence of the antibody moiety, or it can be introduced into the antibody moiety, e.g., by DNA recombinant technology (e.g., by introducing a cysteine or lysine residue into the amino acid sequence) or by protein biochemistry (e.g., by reduction, pH adjustment, or hydrolysis). In some embodiments, the linker is attached to the antibody moiety via a cysteine residue. In some embodiments, the linker is attached to the antibody moiety via a lysine residue.

In some embodiments, the number of drug moieties that can be conjugated to an antibody moiety is limited by the number of free cysteine residues. For example, where the attachment is a cysteine thiol group, an antibody may have only one or a few cysteine thiol groups, or may have only one or a few sufficiently reactive thiol groups through which a linker may be attached. Generally, antibodies do not contain many free and reactive cysteine thiol groups that may be linked to a drug moiety. Indeed, most cysteine thiol residues in antibodies exist as disulfide bridges. Over-attachment of linker-toxin to an antibody may destabilize the antibody by reducing the cysteine residues available to form disulfide bridges. Therefore, an optimal drug:antibody ratio should increase potency of the ADC (by increasing the number of attached drug moieties per antibody) without destabilizing the antibody moiety. In some embodiments, an optimal ratio may be about 1, 2, 4, 7, or 11.

In some embodiments, one or more site-specific conjugation technologies are used to attach an ADC, e.g., to produce a homogeneous ADC product with a defined drug loading, i.e., a defined drug-to-antibody ratio (DAR). In some embodiments, free cysteine residues can be generated in the light chain or heavy chain of antibodies for site-specific conjugation via Residue-SPEcific Conjugation Technology (RESPECT). Exemplary protocols for the generation of RESPECT-formatted antibodies are described in Albone et al. (2017) Cancer Biol. Ther. 18(5):347-57, and in Intl. Pub. Nos. WO/2016205618 and WO/2017106643, each of which is incorporated herein by reference for methods of performing site-specific conjugation. In some embodiments, an ADC is produced using site-specific conjugation to covalently attach an antibody moiety to a drug moiety via a linker (e.g., a linker-payload conjugate disclosed herein). In some embodiments, site-specific conjugation is used to target a DAR of about 2 for ADCs or compositions comprising a compound disclosed herein, e.g., a compound of Table 13, e.g., Compound 1.

In some embodiments, a linker attached to an antibody moiety through a Mal or MC moiety may provide a ratio of about 2, 4, 7, or 11. In some embodiments, an ADC comprising MC-Val-Ala-pABC-Unit 1-Compound 1 joined to an anti-CD73 antibody as disclosed herein has a ratio of about 2, 4, 7, or 11. In some embodiments, an ADC comprising MC-Val-Ala-pABC-Unit 2-Compound 1 joined to an anti-CD73 antibody as disclosed herein has a ratio of about 2, 4, 7, or 11. In some embodiments, an ADC comprising MC-Val-Ala-pABC-Unit 3-Compound 1 joined to an anti-CD73 antibody as disclosed herein has a ratio of about 2, 4, 7, or 11.

In some embodiments, an antibody moiety is exposed to reducing conditions prior to conjugation in order to generate one or more free cysteine residues. An antibody, in some embodiments, may be reduced with a reducing agent such as dithiothreitol (DTT) or tris(2-carboxyethyl)phosphine (TCEP), under partial or total reducing conditions, to generate reactive cysteine thiol groups. Unpaired cysteines may be generated through partial reduction with limited molar equivalents of TCEP, which preferentially reduces the interchain disulfide bonds which link the light chain and heavy chain (one pair per H-L pairing) and the two heavy chains in the hinge region (two pairs per H—H pairing in the case of human IgG1) while leaving the intrachain disulfide bonds intact. See, e.g., Stefano et al. (2013) Methods Mol. Biol. 1045:145-71. In some embodiments, disulfide bonds within the antibodies are reduced electrochemically, e.g., by employing a working electrode that applies an alternating reducing and oxidizing voltage. This approach can allow for on-line coupling of disulfide bond reduction to an analytical device (e.g., an electrochemical detection device, an NMR spectrometer, or a mass spectrometer) or a chemical separation device (e.g., a liquid chromatograph (e.g., an HPLC) or an electrophoresis device). See, e.g., U.S. Publ. No. 20140069822. In certain embodiments, an antibody is subjected to denaturing conditions to reveal reactive nucleophilic groups on amino acid residues, such as lysine or cysteine.

The drug loading of an ADC may be controlled in different ways, e.g., by: (i) limiting the molar excess of drug-linker intermediate or linker reagent relative to antibody; (ii) limiting the conjugation reaction time or temperature; (iii) partial or limiting reductive conditions for cysteine thiol modification; and/or (iv) engineering by recombinant techniques the amino acid sequence of the antibody such that the number and position of cysteine or lysine residues is modified for control of the number and/or position of linker-drug attachments.

In some embodiments, free cysteine residues are introduced into the amino acid sequence of the antibody moiety. For example, cysteine engineered antibodies can be prepared wherein one or more amino acids of a parent antibody are replaced with a cysteine amino acid. Any form of antibody may be so engineered, i.e., mutated. For example, a parent Fab antibody fragment may be engineered to form a cysteine engineered Fab referred to as a “ThioFab.” Similarly, a parent monoclonal antibody may be engineered to form a “ThioMab.” A single site mutation yields a single engineered cysteine residue in a ThioFab, whereas a single site mutation yields two engineered cysteine residues in a ThioMab, due to the dimeric nature of the IgG antibody. DNA encoding an amino acid sequence variant of the parent polypeptide can be prepared by a variety of methods known in the art. See, e.g., the methods described in WO2006/034488. These methods include, but are not limited to, preparation by site-directed (or oligonucleotide-mediated) mutagenesis, PCR mutagenesis, and cassette mutagenesis of an earlier prepared DNA encoding the polypeptide. Variants of recombinant antibodies may also be constructed by restriction fragment manipulation or by overlap extension PCR with synthetic oligonucleotides. ADCs of Formula I include, but are not limited to, antibodies that have 1, 2, 3, or 4 engineered cysteine amino acids. See Lyon et al. (2012) Methods Enzymol. 502:123-38. In some embodiments, one or more free cysteine residues are already present in an antibody moiety, without the use of engineering, in which case the existing free cysteine residues may be used to conjugate the antibody moiety to a drug moiety.

Where more than one nucleophilic group reacts with a drug-linker intermediate or a linker moiety reagent followed by a drug moiety reagent, in a reaction mixture comprising multiple copies of the antibody moiety and linker moiety, then the resulting product can be a mixture of ADC compounds with a distribution of one or more drug moieties attached to each copy of the antibody moiety in the mixture. In some embodiments, the drug loading in a mixture of ADCs resulting from a conjugation reaction ranges from 1 to 12 drug moieties attached per antibody moiety. The average number of drug moieties per antibody moiety (i.e., the average drug loading, or average p) may be calculated by any conventional method known in the art, e.g., by mass spectrometry (e.g., reverse-phase LC-MS), and/or high-performance liquid chromatography (e.g., HPLC). In some embodiments, the average number of drug moieties per antibody moiety is determined by hydrophobic interaction chromatography-high performance liquid chromatography (HIC-HPLC). In some embodiments, the average number of drug moieties per antibody moiety is determined by reverse-phase liquid chromatography-mass spectrometry (LC-MS). In some embodiments, the average number of drug moieties per antibody moiety is from about 1 to about 11; from about 1 to about 8; from about 1 to about 7; from about 1 to about 4; or from about 1 to about 2. In some embodiments, the average number of drug moieties per antibody moiety is about 1. In some embodiments, the average number of drug moieties per antibody moiety is about 2. In some embodiments, the average number of drug moieties per antibody moiety is about 4. In some embodiments, the average number of drug moieties per antibody moiety is about 7. In some embodiments, the average number of drug moieties per antibody moiety is about 11.

Individual ADC compounds having particular DAR ratios, or “species,” may be identified in the mixture, e.g., by mass spectroscopy and separated, e.g., by ultra-performance liquid chromatography (UPLC) or HPLC, e.g., HIC-HPLC. In certain embodiments, a homogeneous or nearly homogenous ADC with a single loading value may be isolated from the conjugation mixture, e.g., by electrophoresis or chromatography.

The present disclosure includes methods of producing the described ADCs. Briefly, the ADCs comprise an antibody or antigen-binding fragment as the antibody moiety, a drug moiety, and a linker that joins the drug moiety and the antibody moiety. In some embodiments, the ADCs can be prepared using a linker having reactive functionalities for covalently attaching to the drug moiety and to the antibody moiety. For example, in some embodiments, a cysteine thiol of an antibody moiety can form a bond with a reactive functional group of a linker or a drug-linker intermediate (e.g., a Mal or MC moiety), the linker or linker intermediate comprising a reactive group that can be conjugated to a functional agent (e.g., a cleavable peptide comprising Compound 1).

In some embodiments, an ADC is produced by contacting an antibody or antigen-binding fragment with a linker and a drug moiety in a sequential manner, such that the antibody moiety is covalently linked to the linker first, and then the pre-formed antibody-linker intermediate reacts with the drug moiety. The antibody-linker intermediate may or may not be subjected to a purification step prior to contacting the drug moiety. In other embodiments, an ADC is produced by contacting an antibody moiety with a linker-drug conjugate, or a salt thereof, pre-formed by reacting a linker with a drug moiety. The pre-formed linker-drug conjugate may or may not be subjected to a purification step prior to contacting the antibody moiety. In other embodiments, the antibody moiety contacts the linker and the drug moiety in one reaction mixture, allowing simultaneous formation of the covalent bonds between the antibody moiety and the linker, and between the linker and the drug moiety. In some embodiments, an ADC is produced by reacting an antibody moiety with a linker joined to a drug moiety, such as LP2, or a salt thereof, under conditions that allow conjugation. In some embodiments, an ADC is produced by reacting an antibody moiety with a linker joined to a drug moiety, such as LP16, or a salt thereof, under conditions that allow conjugation. In some embodiments, an ADC is produced by reacting an antibody moiety with a linker joined to a drug moiety, such as LP20, or a salt thereof, under conditions that allow conjugation. In some embodiments, an ADC is produced by reacting an antibody moiety with a linker joined to a drug moiety, such as LP54, or a salt thereof, under conditions that allow conjugation. In some embodiments, an ADC is produced by reacting an antibody moiety with a linker joined to a drug moiety, such as LP55, or a salt thereof, under conditions that allow conjugation. The conditions that allow conjugation may involve any biochemical methods known in the art for conjugating an ADC. These conditions include, but are not limited to, incubation at room temperature in a suitable buffer (e.g., 1×DBPS, 0.1 M Tris-Glycine at pH 7.4, 10% propylene glycol:90% 1×DPBS, or 1×DPBS, 2 mM EDTA). The conjugation conditions may or may not include the presence of an enzyme (e.g., transglutaminase).

The ADCs prepared according to the methods described above may be subjected to one or more purification steps. The purification step may involve any biochemical methods known in the art for purifying proteins, or any combination of methods thereof. These include, but are not limited to, tangential flow filtration (TFF), affinity chromatography, ion exchange chromatography, any charge or isoelectric point-based chromatography, mixed mode chromatography, e.g., CHT (ceramic hydroxyapatite), hydrophobic interaction chromatography, size exclusion chromatography, dialysis, filtration, selective precipitation, desalting chromatography, or any combination thereof.

### Therapeutic Uses

Disclosed herein are methods of using the disclosed antibodies and/or ADCs in treating a subject for a disorder, e.g., an oncologic disorder. Antibodies and/or ADCs may be administered alone or in combination with one or more additional therapeutic agent(s), and may be administered in any pharmaceutically acceptable formulation, dosage, and/or dosing regimen. Treatment efficacy may be evaluated for toxicity as well as indicators of efficacy and adjusted accordingly. Efficacy measures include, but are not limited to, a cytostatic and/or cytotoxic effect observed in vitro or in vivo, reduced tumor volume, tumor growth inhibition, and/or prolonged survival.

Methods of determining whether an antibody or ADC exerts a cytostatic and/or cytotoxic effect on a cell are known. For example, the cytotoxic activity of an antibody or ADC can be measured by: exposing mammalian cells expressing a target protein of the antibody or ADC (e.g., CD73) in a cell culture medium; co-culturing the cells with immune cells (e.g., myeloid cells such as macrophages) for a period of time, e.g., from about 6 hours to about 5 days; and measuring cell viability. Cell-based in vitro assays may also be used to measure viability (proliferation), cytotoxicity, growth inhibition, and induction of apoptosis (caspase activation) of the antibody or ADC.

In some embodiments, to determine cytotoxicity, phagocytosis, necrosis, or apoptosis (programmed cell death) may be measured. Phagocytosis may be observed using flow cytometry or microscopy. Necrosis is typically accompanied by increased permeability of the plasma membrane, swelling of the cell, and rupture of the plasma membrane. Apoptosis is typically characterized by membrane blebbing, condensation of cytoplasm, and the activation of endogenous endonucleases. Determination of any of these effects on cancer cells indicates that an antibody or ADC is useful in the treatment of cancers.

Cell viability may be measured, e.g., by determining in a cell the uptake of a dye such as neutral red, trypan blue, Crystal Violet, or ALAMAR™ blue. See, e.g., Page et al. (1993) Intl. J. Oncology 3:473-6. In such an assay, the cells are incubated in media containing the dye, the cells are washed, and the remaining dye, reflecting cellular uptake of the dye, is measured spectrophotometrically. In certain embodiments, in vitro potency of prepared antibodies or ADCs is assessed using a Crystal Violet assay. Crystal Violet is a triarylmethane dye that accumulates in the nucleus of viable cells. In this assay, cells are exposed to the antibodies or ADCs or control agents for a defined period of time, after which cells are stained with Crystal Violet, washed copiously with water, then solubilized with 1% SDS and read spectrophotometrically. The protein-binding dye sulforhodamine B (SRB) can also be used to measure cytoxicity. See, e.g., Skehan et al. (1990) J. Natl. Cancer Inst. 82:1107-12.

Apoptosis can be quantified, for example, by measuring DNA fragmentation. Commercial photometric methods for the quantitative in vitro determination of DNA fragmentation are available. Examples of such assays, including TUNEL (which detects incorporation of labeled nucleotides in fragmented DNA) and ELISA-based assays, are described in Biochemica (1999) No. 2, pp. 34-37 (Roche Molecular Biochemicals).

Apoptosis may also be determined by measuring morphological changes in a cell. For example, as with necrosis, loss of plasma membrane integrity can be determined by measuring uptake of certain dyes (e.g., a fluorescent dye such as acridine orange or ethidium bromide). A method for measuring apoptotic cell number has been described by Duke and Cohen, Current Protocols in Immunology. See Coligan et al., eds. (1992) pp. 3.17.1-3.17.16. Cells also can be labeled with a DNA dye (e.g., acridine orange, ethidium bromide, or propidium iodide) and the cells observed for chromatin condensation and margination along the inner nuclear membrane. Other morphological changes that can be measured to determine apoptosis include, e.g., cytoplasmic condensation, increased membrane blebbing, and cellular shrinkage.

In some embodiments, the present disclosure provides a method of killing, or inhibiting or modulating the growth of, a cancer cell or tissue by agonizing the STING pathway and targeting that agonism to particular cells, e.g., cancer cells expressing CD73 (e.g., increased expression level of CD73 relative to non-cancer cells). In some embodiments, agonizing the STING pathway boosts antitumor immunity, e.g., by activating myeloid cells (e.g., macrophages or dendritic cells), cytotoxic T cells, and/or type 1 T helper cell (Th1)-biased responses. The method may be used with any subject where boosting antitumor immunity provides a therapeutic benefit. The antitumor immune response may target a cancer cell regardless of CD73 expression levels.

In various embodiments, the disclosed antibodies and/or ADCs may be administered to affect any cell or tissue that expresses CD73, such as a CD73-expressing cancer cell or tissue. An exemplary embodiment comprises a method of killing a cell via systemic delivery of a STING agonist, e.g., Compound 1, in an anti-CD73 ADC. The method may be used with any cell or tissue that expresses CD73, such as a cancerous cell or metastatic lesion. Non-limiting examples of CD73-expressing cancers include melanoma, diffuse large B-cell lymphoma, T-cell lymphoma, breast cancer, ovarian cancer, head and neck cancer, head and neck squamous carcinoma, non-small-cell lung cancer (NSCLC), glioblastoma, thyroid carcinoma, pancreatic cancer, pancreatic ductal adenocarcinoma (PDAC), esophageal cancer, cervical cancer, gastric cancer, gallbladder cancer, or colorectal cancer. Non-limiting examples of CD73-expressing cells include NCI-H2110 NSCLC cells, MDA-MB-231 breast cancer cells, Ca-OV-3-T2 ovarian cancer cells, HCC1954 breast cancer cells, and cells comprising a recombinant nucleic acid encoding CD73 or a portion thereof. Without being bound by theory, the anti-CD73 antibodies and ADCs disclosed herein may be particularly effective at treating CD73-expressing cancers by targeting CD73-expressing cells for immune clearance, by activating type I or type II interferons and other inflammatory cytokines (e.g., IFN-α, IFN-β, IFN-δ, IFN-ε, IFN-κ, IFN-ω, TNFα, CXCL10, and/or IL-6), and/or by delivering a drug payload (e.g., Compound 1) to cells.

In some embodiments, an ADC may be used to deliver a drug payload (e.g., Compound 1) to cells, wherein the drug payload activates the STING pathway. Without being bound by theory, STING pathway activation may result in the activation of interferons (e.g., type I, type II, type III) and other inflammatory cytokines (e.g., IFN-α, IFN-β, IFN-δ, IFN-ε, IFN-κ, IFN-ω, TNFα, CXCL10, and/or IL-6). In some embodiments, administration of the anti-CD73 ADCs disclosed herein increases expression and/or secretion of IFN-β. In some embodiments, administration of the anti-CD73 ADCs disclosed herein increases expression and/or secretion of TNFα. In some embodiments, administration of the anti-CD73 ADCs disclosed herein increases expression and/or secretion of CXCL10. In some embodiments, administration of the anti-CD73 ADCs disclosed herein increases expression and/or secretion of IL-6. The activation of type I interferons and other inflammatory cytokines may stimulate antitumor immune response by activating dendritic cells and proinflammatory (M1) macrophages, by promoting the generation of cytotoxic T cell responses, and/or by promoting the generation of type 1 T helper cell (Th1)-biased responses. The cancer cell or tumor may then be targeted for killing by these activated immune cells. Activated dendritic cells and proinflammatory (M1) macrophages may also produce type I interferons and other inflammatory cytokines or chemokines, thus enhancing the antitumor inflammatory response. Non-limiting examples of macrophage cells include J774A.1, THP-1, bone marrow-derived macrophages (BMDM), human monocyte derived macrophages (HMDM), and peripheral blood mononuclear cells (PBMC).

In some embodiments, the anti-CD73 antibodies and antigen-binding fragments disclosed herein provide for stable systemic delivery of a STING agonist to a cancer cell or tissue. In some embodiments, the STING agonist is Compound 1. In some embodiments, the cancer cell or tissue expresses CD73. In some embodiments, the cancer cell or tissue is a breast cancer. In some embodiments, the cancer cell or tissue is an ovarian cancer. In some embodiments, the cancer cell or tissue is a PDAC. In some embodiments, the cancer cell or tissue is an NSCLC.

Exemplary methods disclosed herein include the steps of contacting a cell with an antibody and/or ADC as described herein (e.g., by administering the antibody and/or ADC to a subject by a suitable route of administration), in an effective amount, e.g., an amount sufficient to stimulate STING activity. The method can be used on cells in culture, e.g., in vitro, ex vivo, or in situ. For example, cells that express CD73 (e.g., cells collected by biopsy of a tumor or metastatic lesion; cells from an established cancer cell line; or recombinant cells), can be cultured in vitro in culture medium and the contacting step can be affected by adding the antibody and/or ADC to the culture medium. In cells co-cultured with immune cells (e.g., macrophages or dendritic cells), the method will result in killing of cells expressing CD73, including in particular tumor cells expressing CD73. Alternatively, the antibody and/or ADC can be administered to a subject by any suitable administration route (e.g., intravenous, subcutaneous, or direct contact with a tumor tissue) to have an effect in vivo.

The in vivo effect of a disclosed antibody and/or ADC can be evaluated in a suitable animal model. For example, xenogenic cancer models can be used, wherein cancer explants or passaged xenograft cells or tissues are introduced into immune compromised animals, such as nude or SCID mice. See, e.g., Klein et al. (1997) Nature Med. 3:402-8. Efficacy may be predicted using assays that measure inhibition of tumor formation, tumor regression or metastasis, and the like. In some embodiments, the anti-CD73 antibodies and ADCs disclosed herein are more efficacious at inhibiting tumor growth compared to xenograft-bearing mice treated with alternate treatments.

Assays that measure the expression of CD73 and/or cytokines may also be used. Any method for measuring CD73 and/or cytokine expression known in the art may be used, including ELISA (enzyme-linked immunosorbent assay), q-PCR (quantitative polymerase chain reaction), Meso Scale Discovery V-PLEX Cytokine Panel, immunohistochemistry, RNA-seq (RNA-sequencing), Western blot, and flow cytometry. The expression of CD73 may be determined in cancer cells isolated from a subject. In some embodiments, the expression of CD73 is determined prior to administration of an antibody and/or ADC as disclosed herein. In some embodiments, the expression of CD73 is elevated relative to the expression of CD73 in non-cancerous and/or wild-type tissue or cells. In some embodiments, expression of interferons (e.g., type I, type II, type III) and other inflammatory cytokines (e.g., IFN-α, IFN-β, IFN-δ, IFN-ε, IFN-κ, IFN-ω, TNFα, CXCL10, IL-6) is measured. The expression of these cytokines in the tumors of treated subjects may provide an indication of the stimulation of antitumor immune response in response to a treatment.

In various embodiments provided herein are methods of treating CD73-expressing cancer. The antibodies and ADCs disclosed herein can be administered to a non-human mammal or human subject for any therapeutic purposes and via any suitable administration route. The therapeutic methods may entail administering to a mammal having a tumor, e.g., a tumor expressing CD73, a biologically effective amount of an antibody disclosed herein or an ADC comprising a selected chemotherapeutic agent (e.g., Compound 1) linked to that antibody.

In some embodiments, a method of treating a patient having or at risk of having a cancer that expresses CD73 is provided, comprising administering to the patient a therapeutically effective amount of an antibody and/or ADC of the present disclosure. In some embodiments, the patient is non-responsive or poorly responsive to treatment with a drug moiety (e.g., Compound 1) when administered alone, and the patient is administered an antibody or ADC disclosed herein. In other embodiments, the patient is intolerant to treatment with a drug moiety (e.g., Compound 1) when administered alone. For instance, to treat a cancer, a patient may require doses of Compound 1 that lead to systemic toxicity, which are overcome by targeted delivery of the antibodies and/or ADCs disclosed herein to a CD73-expressing cancer, thereby reducing off-target killing. In some embodiments, the patient has a cancer that is inaccessible to local injection of a drug moiety (e.g., Compound 1).

In various embodiments, the methods disclosed herein treat melanoma, diffuse large B-cell lymphoma, breast cancer, ovarian cancer, head and neck cancer, head and neck squamous carcinoma, NSCLC, glioblastoma, thyroid carcinoma, pancreatic cancer, gastric cancer, gallbladder cancer, or colorectal cancer. In some embodiments, the methods disclosed herein treat breast cancer, ovarian cancer, or NSCLC.

The antibodies and/or ADCs of the present disclosure may be administered to a non-human mammal expressing CD73 for veterinary purposes or as an animal model of human disease. Regarding the latter, such animal models may be useful for evaluating the therapeutic efficacy of the disclosed antibodies and ADCs (e.g., testing of dosages and time courses of administration).

In some embodiments, the efficacy of an antibody or ADC may be evaluated by contacting a tumor sample from a subject with the antibody or ADC and evaluating tumor growth rate or volume. In some embodiments, when an antibody or ADC has been determined to be effective, it may be administered to the subject. In some embodiments, the efficacy of an antibody or ADC may be evaluated by contacting a subject with the antibody or ADC and monitoring tumor growth rate or volume. In some embodiments, the efficacy of an antibody or ADC may be evaluated by contacting a subject with the antibody or ADC and monitoring expression of interferons (e.g., type I, type II, type III) and other inflammatory cytokines (e.g., IFN-α, IFN-β, IFN-δ, IFN-ε, IFN-κ, IFN-ω, TNFα, CXCL10, IL-6).

The antibodies and ADCs disclosed herein may be administered at a suitable dosage to a patient in need thereof. Dosages and administration protocols for the treatment of cancers using the foregoing methods will vary with the method and the target cancer, and will generally depend on a number of other factors appreciated in the art.

In various embodiments, treatment involves single bolus or repeated administration of an antibody or ADC preparation via an acceptable route of administration.

The above therapeutic approaches can also be combined with any one of a wide variety of additional surgical, chemotherapy, or radiation therapy regimens. In some embodiments, the above therapeutic approaches are combined with a cancer immunotherapy, e.g., immune checkpoint therapy (e.g., PD-1/PD-L1 inhibitors and CTLA4 inhibitors) or adoptive T cell (ATC) therapy (e.g., chimeric antigen receptor (CAR) T cells).

Further provided herein are therapeutic uses of the disclosed antibodies and/or ADCs. An exemplary embodiment is the use of an antibody and/or ADC in the treatment of a CD73-expressing cancer, such as melanoma, diffuse large B-cell lymphoma, breast cancer, ovarian cancer, head and neck cancer, head and neck squamous carcinoma, NSCLC, glioblastoma, thyroid carcinoma, pancreatic cancer, gastric cancer, gallbladder cancer, or colorectal cancer. Methods for identifying subjects having cancers that express CD73 are known in the art and may be used to identify suitable patients for treatment with a disclosed antibody or ADC.

Another exemplary embodiment is the use of an ADC or an antibody or antigen-binding fragment as disclosed herein in the manufacture of a medicament for the treatment of a CD73-expressing cancer, such as melanoma, diffuse large B-cell lymphoma, T-cell lymphoma, breast cancer, ovarian cancer, head and neck cancer, head and neck squamous carcinoma, non-small-cell lung cancer (NSCLC), glioblastoma, thyroid carcinoma, pancreatic cancer, pancreatic ductal adenocarcinoma (PDAC), esophageal cancer, cervical cancer, gastric cancer, gallbladder cancer, or colorectal cancer.

### Pharmaceutical Compositions and Formulations

An antibody or ADC used in the practice of the foregoing methods may be formulated into a pharmaceutical composition suitable for administration to a subject, e.g., a human subject. In some embodiments, the pharmaceutical composition comprises the antibody and/or ADC and a pharmaceutically acceptable carrier suitable for the desired delivery method. Suitable carriers include any material that, when combined with an antibody or ADC disclosed herein, allows that antibody or ADC to retain its antitumor function and is generally non-reactive with the patient's immune system. Pharmaceutically acceptable carriers may include any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol, mesylate salt, and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives, or buffers, which enhance the shelf life or effectiveness of the ADC.

The pharmaceutical compositions described herein may be in a variety of forms. These include, for example, liquid, semi-solid, and solid dosage forms, such as liquid solutions (e.g., injectable and infusible solutions), dispersions or suspensions, tablets, pills, powders, liposomes, and suppositories. The preferred form depends on the intended mode of administration and therapeutic application.

Pharmaceutical compositions may be solubilized and administered via any route capable of delivering the composition to the tumor site. Potentially effective routes of administration include, but are not limited to, intravenous, parenteral, intraperitoneal, intramuscular, intratumor, intradermal, intraorgan, orthotopic, and the like. Pharmaceutical compositions can be lyophilized and stored as sterile powders, preferably under vacuum, and then reconstituted in bacteriostatic water (containing, for example, benzyl alcohol preservative) or in sterile water prior to injection. Administration can be either systemic or local. Pharmaceutical compositions may comprise an antibody and/or ADC or a pharmaceutically acceptable salt thereof, e.g., a mesylate salt.

In various embodiments, kits for use in the laboratory and the therapeutic applications described herein are within the scope of the present disclosure. Such kits may comprise an antibody or ADC disclosed herein and a carrier, package, or container. The carrier, package, or container may be compartmentalized to receive one or more containers such as vials, tubes, and the like, each of the container(s) comprising one of the separate elements to be used in a method disclosed herein, and/or a label or insert comprising instructions for use, such as a use described herein. Kits may further comprise one or more other containers associated therewith that comprise materials desirable from a commercial and user standpoint, including buffers, diluents, filters, needles, syringes, carrier, package, container, vial and/or tube labels listing contents and/or instructions for use, and package inserts with instructions for use.

A label may be present on or with the container to indicate that the composition is used for a specific therapy or non-therapeutic application, such as a prognostic, prophylactic, diagnostic, or laboratory application. A label may also indicate directions for either in vivo or in vitro use, such as those described herein. Directions and/or other information may also be included on an insert(s) or label(s) which is included with or on the kit. The label may be on or associated with the container. A label may be on a container when letters, numbers, or other characters forming the label are molded or etched into the container itself. A label may be associated with a container when it is present within a receptacle or carrier that also holds the container, e.g., as a package insert. The label may indicate that the composition is used for diagnosing or treating a condition, such as a cancer as described herein.

### EXEMPLARY EMBODIMENTS OF THE DISCLOSURE

In various embodiments, the present disclosure provides novel antibody-drug conjugates comprising an antibody or antigen-binding fragment disclosed herein that is capable of specifically binding CD73.

In some embodiments, the antibody-drug conjugate comprises any anti-CD73 antibody or antigen-binding fragment disclosed herein, a linker comprising MC-Val-Ala-pABC-Unit 1 moiety, and a drug moiety comprising a compound selected from a compound of Table 13.

In some embodiments, the antibody-drug conjugate comprises any anti-CD73 antibody or antigen-binding fragment disclosed herein and a linker-drug conjugate comprising MC-Val-Ala-pABC-Unit 1-Compound 1. In some embodiments, the antibody-drug conjugate comprises any anti-CD73 antibody or antigen-binding fragment disclosed herein conjugated to LP2.

In some embodiments, the antibody-drug conjugate comprises any anti-CD73 antibody or antigen-binding fragment disclosed herein, a linker comprising MC-Val-Ala-pABC-Unit 2 moiety, and a drug moiety comprising a compound selected from a compound of Table 13.

In some embodiments, the antibody-drug conjugate comprises any anti-CD73 antibody or antigen-binding fragment disclosed herein and a linker-drug conjugate comprising MC-Val-Ala-pABC-Unit 2-Compound 1. In some embodiments, the antibody-drug conjugate comprises any anti-CD73 antibody or antigen-binding fragment disclosed herein conjugated to LP16.

In some embodiments, the antibody-drug conjugate comprises any anti-CD73 antibody or antigen-binding fragment disclosed herein, a linker comprising MC-Val-Ala-pABC-Unit 3 moiety, and a drug moiety comprising a compound selected from a compound of Table 13.

In some embodiments, the antibody-drug conjugate comprises any anti-CD73 antibody or antigen-binding fragment disclosed herein and a linker-drug conjugate comprising MC-Val-Ala-pABC-Unit 3-Compound 1. In some embodiments, the antibody-drug conjugate comprises any anti-CD73 antibody or antigen-binding fragment disclosed herein conjugated to LP20.

In some embodiments, the antibody-drug conjugate comprises any anti-CD73 antibody or antigen-binding fragment disclosed herein, a linker comprising Mal-Formula (II)-Val-Ala-pABC-Unit 2 moiety, and a drug moiety comprising a compound selected from a compound of Table 13.

In some embodiments, the antibody-drug conjugate comprises any anti-CD73 antibody or antigen-binding fragment disclosed herein and a linker-drug conjugate comprising Mal-Formula (II)-Val-Ala-pABC-Unit 2-Compound 1. In some embodiments, the antibody-drug conjugate comprises any anti-CD73 antibody or antigen-binding fragment disclosed herein conjugated to LP54.

In some embodiments, the antibody-drug conjugate comprises any anti-CD73 antibody or antigen-binding fragment disclosed herein, a linker comprising Mal-(PEG)₂-Val-Ala-pABC-Unit 2 moiety, and a drug moiety comprising a compound selected from a compound of Table 13.

In some embodiments, the antibody-drug conjugate comprises any anti-CD73 antibody or antigen-binding fragment disclosed herein and a linker-drug conjugate comprising Mal-(PEG)₂-Val-Ala-pABC-Unit 2-Compound 1. In some embodiments, the antibody-drug conjugate comprises any anti-CD73 antibody or antigen-binding fragment disclosed herein conjugated to LP55.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 1, HCDR2 comprising SEQ ID NO: 2, HCDR3 comprising SEQ ID NO: 3; LCDR1 comprising SEQ ID NO: 4, LCDR2 comprising SEQ ID NO: 5, and LCDR3 comprising SEQ ID NO: 6, as defined by the Kabat numbering system; any linker disclosed herein; and a drug moiety comprising a compound selected from a compound of Table 13. In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 7, HCDR2 comprising SEQ ID NO: 8, HCDR3 comprising SEQ ID NO: 9; LCDR1 comprising SEQ ID NO: 10, LCDR2 comprising SEQ ID NO: 11, and LCDR3 comprising SEQ ID NO: 12, as defined by the IGMT numbering system; any linker disclosed herein; and a drug moiety comprising a compound selected from a compound of Table 13.

In some embodiments, the antibody-drug conjugate comprises an anti-CD73 antibody or antigen-binding fragment comprising a heavy chain variable region comprising an amino acid sequence of SEQ ID NO: 49, and a light chain variable region comprising an amino acid sequence of SEQ ID NO: 50; any linker disclosed herein; and a drug moiety comprising a compound selected from a compound of Table 13.

In some embodiments, the antibody-drug conjugate comprises an anti-CD73 antibody or antigen-binding fragment comprising a heavy chain variable region comprising an amino acid sequence of SEQ ID NO: 49, and a light chain variable region comprising an amino acid sequence of SEQ ID NO: 51; any linker disclosed herein; and a drug moiety comprising a compound selected from a compound of Table 13.

In some embodiments, the antibody-drug conjugate comprises an anti-CD73 antibody or antigen-binding fragment comprising a heavy chain amino acid sequence of SEQ ID NO: 63 and a light chain amino acid sequence of SEQ ID NO: 65; any linker disclosed herein; and a drug moiety comprising a compound selected from a compound of Table 13.

In some embodiments, the antibody-drug conjugate comprises an anti-CD73 antibody or antigen-binding fragment comprising a heavy chain amino acid sequence of SEQ ID NO: 64 and a light chain amino acid sequence of SEQ ID NO: 65; any linker disclosed herein; and a drug moiety comprising a compound selected from a compound of Table 13.

In some embodiments, the antibody-drug conjugate comprises an anti-CD73 antibody or antigen-binding fragment comprising a heavy chain amino acid sequence of SEQ ID NO: 63 and a light chain amino acid sequence of SEQ ID NO: 66; any linker disclosed herein; and a drug moiety comprising a compound selected from a compound of Table 13.

In some embodiments, the antibody-drug conjugate comprises an anti-CD73 antibody or antigen-binding fragment comprising a heavy chain amino acid sequence of SEQ ID NO: 64 and a light chain amino acid sequence of SEQ ID NO: 66; any linker disclosed herein; and a drug moiety comprising a compound selected from a compound of Table 13.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 1, HCDR2 comprising SEQ ID NO: 2, HCDR3 comprising SEQ ID NO: 3; LCDR1 comprising SEQ ID NO: 4, LCDR2 comprising SEQ ID NO: 5, and LCDR3 comprising SEQ ID NO: 6, as defined by the Kabat numbering system; any linker disclosed herein; and a drug moiety comprising Compound 1. In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 7, HCDR2 comprising SEQ ID NO: 8, HCDR3 comprising SEQ ID NO: 9; LCDR1 comprising SEQ ID NO: 10, LCDR2 comprising SEQ ID NO: 11, and LCDR3 comprising SEQ ID NO: 12, as defined by the IGMT numbering system; any linker disclosed herein; and a drug moiety comprising Compound 1.

In some embodiments, the antibody-drug conjugate comprises an anti-CD73 antibody or antigen-binding fragment comprising a heavy chain variable region comprising an amino acid sequence of SEQ ID NO: 49, and a light chain variable region comprising an amino acid sequence of SEQ ID NO: 50; any linker disclosed herein; and a drug moiety comprising Compound 1.

In some embodiments, the antibody-drug conjugate comprises an anti-CD73 antibody or antigen-binding fragment comprising a heavy chain variable region comprising an amino acid sequence of SEQ ID NO: 49, and a light chain variable region comprising an amino acid sequence of SEQ ID NO: 51; any linker disclosed herein; and a drug moiety comprising Compound 1.

In some embodiments, the antibody-drug conjugate comprises an anti-CD73 antibody or antigen-binding fragment comprising a heavy chain amino acid sequence of SEQ ID NO: 63 and a light chain amino acid sequence of SEQ ID NO: 65; any linker disclosed herein; and a drug moiety comprising Compound 1.

In some embodiments, the antibody-drug conjugate comprises an anti-CD73 antibody or antigen-binding fragment comprising a heavy chain amino acid sequence of SEQ ID NO: 64 and a light chain amino acid sequence of SEQ ID NO: 65; any linker disclosed herein; and a drug moiety comprising Compound 1.

In some embodiments, the antibody-drug conjugate comprises an anti-CD73 antibody or antigen-binding fragment comprising a heavy chain amino acid sequence of SEQ ID NO: 63 and a light chain amino acid sequence of SEQ ID NO: 66; any linker disclosed herein; and a drug moiety comprising Compound 1.

In some embodiments, the antibody-drug conjugate comprises an anti-CD73 antibody or antigen-binding fragment comprising a heavy chain amino acid sequence of SEQ ID NO: 64 and a light chain amino acid sequence of SEQ ID NO: 66; any linker disclosed herein; and a drug moiety comprising Compound 1.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 1, HCDR2 comprising SEQ ID NO: 2, HCDR3 comprising SEQ ID NO: 3; LCDR1 comprising SEQ ID NO: 4, LCDR2 comprising SEQ ID NO: 5, and LCDR3 comprising SEQ ID NO: 6, as defined by the Kabat numbering system; a linker comprising MC-Val-Ala-pABC-Unit 1 moiety; and a drug moiety comprising Compound 1.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 1, HCDR2 comprising SEQ ID NO: 2, HCDR3 comprising SEQ ID NO: 3; LCDR1 comprising SEQ ID NO: 4, LCDR2 comprising SEQ ID NO: 5, and LCDR3 comprising SEQ ID NO: 6, as defined by the Kabat numbering system; and a linker-drug conjugate comprising LP2.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 7, HCDR2 comprising SEQ ID NO: 8, HCDR3 comprising SEQ ID NO: 9; LCDR1 comprising SEQ ID NO: 10, LCDR2 comprising SEQ ID NO: 11, and LCDR3 comprising SEQ ID NO: 12, as defined by the IGMT numbering system; a linker comprising MC-Val-Ala-pABC-Unit 1 moiety; and a drug moiety comprising Compound 1.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 7, HCDR2 comprising SEQ ID NO: 8, HCDR3 comprising SEQ ID NO: 9; LCDR1 comprising SEQ ID NO: 10, LCDR2 comprising SEQ ID NO: 11, and LCDR3 comprising SEQ ID NO: 12, as defined by the IGMT numbering system; and a linker-drug conjugate comprising LP2.

In some embodiments, the antibody-drug conjugate comprises an anti-CD73 antibody or antigen-binding fragment comprising a heavy chain variable region comprising an amino acid sequence of SEQ ID NO: 49, and a light chain variable region comprising an amino acid sequence of SEQ ID NO: 50; a linker comprising MC-Val-Ala-pABC-Unit 1 moiety; and a drug moiety comprising Compound 1.

In some embodiments, the antibody-drug conjugate comprises an anti-CD73 antibody or antigen-binding fragment comprising a heavy chain variable region comprising an amino acid sequence of SEQ ID NO: 49, and a light chain variable region comprising an amino acid sequence of SEQ ID NO: 50; and a linker-drug conjugate comprising LP2.

In some embodiments, the antibody-drug conjugate comprises an anti-CD73 antibody or antigen-binding fragment comprising a heavy chain variable region comprising an amino acid sequence of SEQ ID NO: 49, and a light chain variable region comprising an amino acid sequence of SEQ ID NO: 51; a linker comprising MC-Val-Ala-pABC-Unit 1 moiety; and a drug moiety comprising Compound 1.

In some embodiments, the antibody-drug conjugate comprises an anti-CD73 antibody or antigen-binding fragment comprising a heavy chain variable region comprising an amino acid sequence of SEQ ID NO: 49, and a light chain variable region comprising an amino acid sequence of SEQ ID NO: 51; and a linker-drug conjugate comprising LP2.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 1, HCDR2 comprising SEQ ID NO: 2, HCDR3 comprising SEQ ID NO: 3; LCDR1 comprising SEQ ID NO: 4, LCDR2 comprising SEQ ID NO: 5, and LCDR3 comprising SEQ ID NO: 6, as defined by the Kabat numbering system; a linker comprising MC-Val-Ala-pABC-Unit 2 moiety; and a drug moiety comprising Compound 1.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 1, HCDR2 comprising SEQ ID NO: 2, HCDR3 comprising SEQ ID NO: 3; LCDR1 comprising SEQ ID NO: 4, LCDR2 comprising SEQ ID NO: 5, and LCDR3 comprising SEQ ID NO: 6, as defined by the Kabat numbering system; and a linker-drug conjugate comprising LP16.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 7, HCDR2 comprising SEQ ID NO: 8, HCDR3 comprising SEQ ID NO: 9; LCDR1 comprising SEQ ID NO: 10, LCDR2 comprising SEQ ID NO: 11, and LCDR3 comprising SEQ ID NO: 12, as defined by the IGMT numbering system; a linker comprising MC-Val-Ala-pABC-Unit 2 moiety; and a drug moiety comprising Compound 1.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 7, HCDR2 comprising SEQ ID NO: 8, HCDR3 comprising SEQ ID NO: 9; LCDR1 comprising SEQ ID NO: 10, LCDR2 comprising SEQ ID NO: 11, and LCDR3 comprising SEQ ID NO: 12, as defined by the IGMT numbering system; and a linker-drug conjugate comprising LP16.

In some embodiments, the antibody-drug conjugate comprises an anti-CD73 antibody or antigen-binding fragment comprising a heavy chain variable region comprising an amino acid sequence of SEQ ID NO: 49, and a light chain variable region comprising an amino acid sequence of SEQ ID NO: 50; a linker comprising MC-Val-Ala-pABC-Unit 2 moiety; and a drug moiety comprising Compound 1.

In some embodiments, the antibody-drug conjugate comprises an anti-CD73 antibody or antigen-binding fragment comprising a heavy chain variable region comprising an amino acid sequence of SEQ ID NO: 49, and a light chain variable region comprising an amino acid sequence of SEQ ID NO: 50; and a linker-drug conjugate comprising LP16.

In some embodiments, the antibody-drug conjugate comprises an anti-CD73 antibody or antigen-binding fragment comprising a heavy chain variable region comprising an amino acid sequence of SEQ ID NO: 49, and a light chain variable region comprising an amino acid sequence of SEQ ID NO: 51; a linker comprising MC-Val-Ala-pABC-Unit 2 moiety; and a drug moiety comprising Compound 1.

In some embodiments, the antibody-drug conjugate comprises an anti-CD73 antibody or antigen-binding fragment comprising a heavy chain variable region comprising an amino acid sequence of SEQ ID NO: 49, and a light chain variable region comprising an amino acid sequence of SEQ ID NO: 51; and a linker-drug conjugate comprising LP16.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 1, HCDR2 comprising SEQ ID NO: 2, HCDR3 comprising SEQ ID NO: 3; LCDR1 comprising SEQ ID NO: 4, LCDR2 comprising SEQ ID NO: 5, and LCDR3 comprising SEQ ID NO: 6, as defined by the Kabat numbering system; a linker comprising MC-Val-Ala-pABC-Unit 3 moiety; and a drug moiety comprising Compound 1.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 1, HCDR2 comprising SEQ ID NO: 2, HCDR3 comprising SEQ ID NO: 3; LCDR1 comprising SEQ ID NO: 4, LCDR2 comprising SEQ ID NO: 5, and LCDR3 comprising SEQ ID NO: 6, as defined by the Kabat numbering system; and a linker-drug conjugate comprising LP20.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 7, HCDR2 comprising SEQ ID NO: 8, HCDR3 comprising SEQ ID NO: 9; LCDR1 comprising SEQ ID NO: 10, LCDR2 comprising SEQ ID NO: 11, and LCDR3 comprising SEQ ID NO: 12, as defined by the IGMT numbering system; a linker comprising MC-Val-Ala-pABC-Unit 3 moiety; and a drug moiety comprising Compound 1.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 7, HCDR2 comprising SEQ ID NO: 8, HCDR3 comprising SEQ ID NO: 9; LCDR1 comprising SEQ ID NO: 10, LCDR2 comprising SEQ ID NO: 11, and LCDR3 comprising SEQ ID NO: 12, as defined by the IGMT numbering system; and a linker-drug conjugate comprising LP20.

In some embodiments, the antibody-drug conjugate comprises an anti-CD73 antibody or antigen-binding fragment comprising a heavy chain variable region comprising an amino acid sequence of SEQ ID NO: 49, and a light chain variable region comprising an amino acid sequence of SEQ ID NO: 50; a linker comprising MC-Val-Ala-pABC-Unit 3 moiety; and a drug moiety comprising Compound 1.

In some embodiments, the antibody-drug conjugate comprises an anti-CD73 antibody or antigen-binding fragment comprising a heavy chain variable region comprising an amino acid sequence of SEQ ID NO: 49, and a light chain variable region comprising an amino acid sequence of SEQ ID NO: 50; and a linker-drug conjugate comprising LP20.

In some embodiments, the antibody-drug conjugate comprises an anti-CD73 antibody or antigen-binding fragment comprising a heavy chain variable region comprising an amino acid sequence of SEQ ID NO: 49, and a light chain variable region comprising an amino acid sequence of SEQ ID NO: 51; a linker comprising MC-Val-Ala-pABC-Unit 3 moiety; and a drug moiety comprising Compound 1.

In some embodiments, the antibody-drug conjugate comprises an anti-CD73 antibody or antigen-binding fragment comprising a heavy chain variable region comprising an amino acid sequence of SEQ ID NO: 49, and a light chain variable region comprising an amino acid sequence of SEQ ID NO: 51; and a linker-drug conjugate comprising LP20.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 1, HCDR2 comprising SEQ ID NO: 2, HCDR3 comprising SEQ ID NO: 3; LCDR1 comprising SEQ ID NO: 4, LCDR2 comprising SEQ ID NO: 5, and LCDR3 comprising SEQ ID NO: 6, as defined by the Kabat numbering system; a linker comprising Mal-Formula (II)-Val-Ala-pABC-Unit 2 moiety; and a drug moiety comprising Compound 1.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 1, HCDR2 comprising SEQ ID NO: 2, HCDR3 comprising SEQ ID NO: 3; LCDR1 comprising SEQ ID NO: 4, LCDR2 comprising SEQ ID NO: 5, and LCDR3 comprising SEQ ID NO: 6, as defined by the Kabat numbering system; and a linker-drug conjugate comprising LP54.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 7, HCDR2 comprising SEQ ID NO: 8, HCDR3 comprising SEQ ID NO: 9; LCDR1 comprising SEQ ID NO: 10, LCDR2 comprising SEQ ID NO: 11, and LCDR3 comprising SEQ ID NO: 12, as defined by the IGMT numbering system; a linker comprising Mal-Formula (II)-Val-Ala-pABC-Unit 2 moiety; and a drug moiety comprising Compound 1.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 7, HCDR2 comprising SEQ ID NO: 8, HCDR3 comprising SEQ ID NO: 9; LCDR1 comprising SEQ ID NO: 10, LCDR2 comprising SEQ ID NO: 11, and LCDR3 comprising SEQ ID NO: 12, as defined by the IGMT numbering system; and a linker-drug conjugate comprising LP54.

In some embodiments, the antibody-drug conjugate comprises an anti-CD73 antibody or antigen-binding fragment comprising a heavy chain variable region comprising an amino acid sequence of SEQ ID NO: 49, and a light chain variable region comprising an amino acid sequence of SEQ ID NO: 50; a linker comprising Mal-Formula (II)-Val-Ala-pABC-Unit 2 moiety; and a drug moiety comprising Compound 1.

In some embodiments, the antibody-drug conjugate comprises an anti-CD73 antibody or antigen-binding fragment comprising a heavy chain variable region comprising an amino acid sequence of SEQ ID NO: 49, and a light chain variable region comprising an amino acid sequence of SEQ ID NO: 50; and a linker-drug conjugate comprising LP54.

In some embodiments, the antibody-drug conjugate comprises an anti-CD73 antibody or antigen-binding fragment comprising a heavy chain variable region comprising an amino acid sequence of SEQ ID NO: 49, and a light chain variable region comprising an amino acid sequence of SEQ ID NO: 51; a linker comprising Mal-Formula (II)-Val-Ala-pABC-Unit 2 moiety; and a drug moiety comprising Compound 1.

In some embodiments, the antibody-drug conjugate comprises an anti-CD73 antibody or antigen-binding fragment comprising a heavy chain variable region comprising an amino acid sequence of SEQ ID NO: 49, and a light chain variable region comprising an amino acid sequence of SEQ ID NO: 51; and a linker-drug conjugate comprising LP54.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 1, HCDR2 comprising SEQ ID NO: 2, HCDR3 comprising SEQ ID NO: 3; LCDR1 comprising SEQ ID NO: 4, LCDR2 comprising SEQ ID NO: 5, and LCDR3 comprising SEQ ID NO: 6, as defined by the Kabat numbering system; a linker comprising Mal-(PEG)₂-Val-Ala-pABC-Unit 2 moiety; and a drug moiety comprising Compound 1.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 1, HCDR2 comprising SEQ ID NO: 2, HCDR3 comprising SEQ ID NO: 3; LCDR1 comprising SEQ ID NO: 4, LCDR2 comprising SEQ ID NO: 5, and LCDR3 comprising SEQ ID NO: 6, as defined by the Kabat numbering system; and a linker-drug conjugate comprising LP55.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 7, HCDR2 comprising SEQ ID NO: 8, HCDR3 comprising SEQ ID NO: 9; LCDR1 comprising SEQ ID NO: 10, LCDR2 comprising SEQ ID NO: 11, and LCDR3 comprising SEQ ID NO: 12, as defined by the IGMT numbering system; a linker comprising (PEG)₂-Val-Ala-pABC-Unit 2 moiety; and a drug moiety comprising Compound 1.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 7, HCDR2 comprising SEQ ID NO: 8, HCDR3 comprising SEQ ID NO: 9; LCDR1 comprising SEQ ID NO: 10, LCDR2 comprising SEQ ID NO: 11, and LCDR3 comprising SEQ ID NO: 12, as defined by the IGMT numbering system; and a linker-drug conjugate comprising LP55.

In some embodiments, the antibody-drug conjugate comprises an anti-CD73 antibody or antigen-binding fragment comprising a heavy chain variable region comprising an amino acid sequence of SEQ ID NO: 49, and a light chain variable region comprising an amino acid sequence of SEQ ID NO: 50; a linker comprising Mal-(PEG)₂-Val-Ala-pABC-Unit 2 moiety; and a drug moiety comprising Compound 1.

In some embodiments, the antibody-drug conjugate comprises an anti-CD73 antibody or antigen-binding fragment comprising a heavy chain variable region comprising an amino acid sequence of SEQ ID NO: 49, and a light chain variable region comprising an amino acid sequence of SEQ ID NO: 50; and a linker-drug conjugate comprising LP55.

In some embodiments, the antibody-drug conjugate comprises an anti-CD73 antibody or antigen-binding fragment comprising a heavy chain variable region comprising an amino acid sequence of SEQ ID NO: 49, and a light chain variable region comprising an amino acid sequence of SEQ ID NO: 51; a linker comprising Mal-(PEG)₂-Val-Ala-pABC-Unit 2 moiety; and a drug moiety comprising Compound 1.

In some embodiments, the antibody-drug conjugate comprises an anti-CD73 antibody or antigen-binding fragment comprising a heavy chain variable region comprising an amino acid sequence of SEQ ID NO: 49, and a light chain variable region comprising an amino acid sequence of SEQ ID NO: 51; and a linker-drug conjugate comprising LP55.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 13, HCDR2 comprising SEQ ID NO: 14, HCDR3 comprising SEQ ID NO: 15; LCDR1 comprising SEQ ID NO: 16, LCDR2 comprising SEQ ID NO: 17, and LCDR3 comprising SEQ ID NO: 18, as defined by the Kabat numbering system; any linker disclosed herein; and a drug moiety comprising a compound selected from a compound of Table 13. In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 19, HCDR2 comprising SEQ ID NO: 20, HCDR3 comprising SEQ ID NO: 21; LCDR1 comprising SEQ ID NO: 22, LCDR2 comprising SEQ ID NO: 23, and LCDR3 comprising SEQ ID NO: 24, as defined by the IMGT numbering system; any linker disclosed herein; and a drug moiety comprising a compound selected from a compound of Table 13.

In some embodiments, the antibody-drug conjugate comprises an anti-CD73 antibody or antigen-binding fragment comprising a heavy chain variable region comprising an amino acid sequence of SEQ ID NO: 52, and a light chain variable region comprising an amino acid sequence of SEQ ID NO: 53; any linker disclosed herein; and a drug moiety comprising a compound selected from a compound of Table 13.

In some embodiments, the antibody-drug conjugate comprises an anti-CD73 antibody or antigen-binding fragment comprising a heavy chain variable region comprising an amino acid sequence of SEQ ID NO: 52, and a light chain variable region comprising an amino acid sequence of SEQ ID NO: 54; any linker disclosed herein; and a drug moiety comprising a compound selected from a compound of Table 13.

In some embodiments, the antibody-drug conjugate comprises an anti-CD73 antibody or antigen-binding fragment comprising a heavy chain amino acid sequence of SEQ ID NO: 69 and a light chain amino acid sequence of SEQ ID NO: 71; any linker disclosed herein; and a drug moiety comprising a compound selected from a compound of Table 13.

In some embodiments, the antibody-drug conjugate comprises an anti-CD73 antibody or antigen-binding fragment comprising a heavy chain amino acid sequence of SEQ ID NO: 70 and a light chain amino acid sequence of SEQ ID NO: 71; any linker disclosed herein; and a drug moiety comprising a compound selected from a compound of Table 13. In some embodiments, the antibody-drug conjugate comprises an anti-CD73 antibody or antigen-binding fragment comprising a heavy chain amino acid sequence of SEQ ID NO: 69 and a light chain amino acid sequence of SEQ ID NO: 72; any linker disclosed herein; and a drug moiety comprising a compound selected from a compound of Table 13.

In some embodiments, the antibody-drug conjugate comprises an anti-CD73 antibody or antigen-binding fragment comprising a heavy chain amino acid sequence of SEQ ID NO: 70 and a light chain amino acid sequence of SEQ ID NO: 72; any linker disclosed herein; and a drug moiety comprising a compound selected from a compound of Table 13. In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 13, HCDR2 comprising SEQ ID NO: 14, HCDR3 comprising SEQ ID NO: 15; LCDR1 comprising SEQ ID NO: 16, LCDR2 comprising SEQ ID NO: 17, and LCDR3 comprising SEQ ID NO: 18, as defined by the Kabat numbering system; any linker disclosed herein; and a drug moiety comprising Compound 1. In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 19, HCDR2 comprising SEQ ID NO: 20, HCDR3 comprising SEQ ID NO: 21; LCDR1 comprising SEQ ID NO: 22, LCDR2 comprising SEQ ID NO: 23, and LCDR3 comprising SEQ ID NO: 24, as defined by the IMGT numbering system; any linker disclosed herein; and a drug moiety comprising Compound 1.

In some embodiments, the antibody-drug conjugate comprises an anti-CD73 antibody or antigen-binding fragment comprising a heavy chain variable region comprising an amino acid sequence of SEQ ID NO: 52, and a light chain variable region comprising an amino acid sequence of SEQ ID NO: 53; any linker disclosed herein; and a drug moiety comprising Compound 1.

In some embodiments, the antibody-drug conjugate comprises an anti-CD73 antibody or antigen-binding fragment comprising a heavy chain variable region comprising an amino acid sequence of SEQ ID NO: 52, and a light chain variable region comprising an amino acid sequence of SEQ ID NO: 54; any linker disclosed herein; and a drug moiety comprising Compound 1.

In some embodiments, the antibody-drug conjugate comprises an anti-CD73 antibody or antigen-binding fragment comprising a heavy chain amino acid sequence of SEQ ID NO: 69 and a light chain amino acid sequence of SEQ ID NO: 71; any linker disclosed herein; and a drug moiety comprising Compound 1.

In some embodiments, the antibody-drug conjugate comprises an anti-CD73 antibody or antigen-binding fragment comprising a heavy chain amino acid sequence of SEQ ID NO: 70 and a light chain amino acid sequence of SEQ ID NO: 71; any linker disclosed herein; and a drug moiety comprising Compound 1.

In some embodiments, the antibody-drug conjugate comprises an anti-CD73 antibody or antigen-binding fragment comprising a heavy chain amino acid sequence of SEQ ID NO: 69 and a light chain amino acid sequence of SEQ ID NO: 72; any linker disclosed herein; and a drug moiety comprising Compound 1.

In some embodiments, the antibody-drug conjugate comprises an anti-CD73 antibody or antigen-binding fragment comprising a heavy chain amino acid sequence of SEQ ID NO: 70 and a light chain amino acid sequence of SEQ ID NO: 72; any linker disclosed herein; and a drug moiety comprising Compound 1.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 13, HCDR2 comprising SEQ ID NO: 14, HCDR3 comprising SEQ ID NO: 15; LCDR1 comprising SEQ ID NO: 16, LCDR2 comprising SEQ ID NO: 17, and LCDR3 comprising SEQ ID NO: 18, as defined by the Kabat numbering system; a linker comprising MC-Val-Ala-pABC-Unit 1 moiety; and a drug moiety comprising Compound 1.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 13, HCDR2 comprising SEQ ID NO: 14, HCDR3 comprising SEQ ID NO: 15; LCDR1 comprising SEQ ID NO: 16, LCDR2 comprising SEQ ID NO: 17, and LCDR3 comprising SEQ ID NO: 18, as defined by the Kabat numbering system; and a linker-drug conjugate comprising LP2.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 19, HCDR2 comprising SEQ ID NO: 20, HCDR3 comprising SEQ ID NO: 21; LCDR1 comprising SEQ ID NO: 22, LCDR2 comprising SEQ ID NO: 23, and LCDR3 comprising SEQ ID NO: 24, as defined by the IMGT numbering system; a linker comprising MC-Val-Ala-pABC-Unit 1 moiety; and a drug moiety comprising Compound 1.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 19, HCDR2 comprising SEQ ID NO: 20, HCDR3 comprising SEQ ID NO: 21; LCDR1 comprising SEQ ID NO: 22, LCDR2 comprising SEQ ID NO: 23, and LCDR3 comprising SEQ ID NO: 24, as defined by the IMGT numbering system; and a linker-drug conjugate comprising LP2.

In some embodiments, the antibody-drug conjugate comprises an anti-CD73 antibody or antigen-binding fragment comprising a heavy chain variable region comprising an amino acid sequence of SEQ ID NO: 52, and a light chain variable region comprising an amino acid sequence of SEQ ID NO: 53; a linker comprising MC-Val-Ala-pABC-Unit 1 moiety; and a drug moiety comprising Compound 1.

In some embodiments, the antibody-drug conjugate comprises an anti-CD73 antibody or antigen-binding fragment comprising a heavy chain variable region comprising an amino acid sequence of SEQ ID NO: 52, and a light chain variable region comprising an amino acid sequence of SEQ ID NO: 53; and a linker-drug conjugate comprising LP2.

In some embodiments, the antibody-drug conjugate comprises an anti-CD73 antibody or antigen-binding fragment comprising a heavy chain variable region comprising an amino acid sequence of SEQ ID NO: 52, and a light chain variable region comprising an amino acid sequence of SEQ ID NO: 54; a linker comprising MC-Val-Ala-pABC-Unit 1 moiety; and a drug moiety comprising Compound 1.

In some embodiments, the antibody-drug conjugate comprises an anti-CD73 antibody or antigen-binding fragment comprising a heavy chain variable region comprising an amino acid sequence of SEQ ID NO: 52, and a light chain variable region comprising an amino acid sequence of SEQ ID NO: 54; and a linker-drug conjugate comprising LP2.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 13, HCDR2 comprising SEQ ID NO: 14, HCDR3 comprising SEQ ID NO: 15; LCDR1 comprising SEQ ID NO: 16, LCDR2 comprising SEQ ID NO: 17, and LCDR3 comprising SEQ ID NO: 18, as defined by the Kabat numbering system; a linker comprising MC-Val-Ala-pABC-Unit 2 moiety; and a drug moiety comprising Compound 1.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 13, HCDR2 comprising SEQ ID NO: 14, HCDR3 comprising SEQ ID NO: 15; LCDR1 comprising SEQ ID NO: 16, LCDR2 comprising SEQ ID NO: 17, and LCDR3 comprising SEQ ID NO: 18, as defined by the Kabat numbering system; and a linker-drug conjugate comprising LP16.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 19, HCDR2 comprising SEQ ID NO: 20, HCDR3 comprising SEQ ID NO: 21; LCDR1 comprising SEQ ID NO: 22, LCDR2 comprising SEQ ID NO: 23, and LCDR3 comprising SEQ ID NO: 24, as defined by the IMGT numbering system; a linker comprising MC-Val-Ala-pABC-Unit 2 moiety; and a drug moiety comprising Compound 1.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 19, HCDR2 comprising SEQ ID NO: 20, HCDR3 comprising SEQ ID NO: 21; LCDR1 comprising SEQ ID NO: 22, LCDR2 comprising SEQ ID NO: 23, and LCDR3 comprising SEQ ID NO: 24, as defined by the IMGT numbering system; and a linker-drug conjugate comprising LP16.

In some embodiments, the antibody-drug conjugate comprises an anti-CD73 antibody or antigen-binding fragment comprising a heavy chain variable region comprising an amino acid sequence of SEQ ID NO: 52, and a light chain variable region comprising an amino acid sequence of SEQ ID NO: 53; a linker comprising MC-Val-Ala-pABC-Unit 2 moiety; and a drug moiety comprising Compound 1.

In some embodiments, the antibody-drug conjugate comprises an anti-CD73 antibody or antigen-binding fragment comprising a heavy chain variable region comprising an amino acid sequence of SEQ ID NO: 52, and a light chain variable region comprising an amino acid sequence of SEQ ID NO: 53; and a linker-drug conjugate comprising LP16.

In some embodiments, the antibody-drug conjugate comprises an anti-CD73 antibody or antigen-binding fragment comprising a heavy chain variable region comprising an amino acid sequence of SEQ ID NO: 52, and a light chain variable region comprising an amino acid sequence of SEQ ID NO: 54; a linker comprising MC-Val-Ala-pABC-Unit 2 moiety; and a drug moiety comprising Compound 1.

In some embodiments, the antibody-drug conjugate comprises an anti-CD73 antibody or antigen-binding fragment comprising a heavy chain variable region comprising an amino acid sequence of SEQ ID NO: 52, and a light chain variable region comprising an amino acid sequence of SEQ ID NO: 54; and a linker-drug conjugate comprising LP16.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 13, HCDR2 comprising SEQ ID NO: 14, HCDR3 comprising SEQ ID NO: 15; LCDR1 comprising SEQ ID NO: 16, LCDR2 comprising SEQ ID NO: 17, and LCDR3 comprising SEQ ID NO: 18, as defined by the Kabat numbering system; a linker comprising MC-Val-Ala-pABC-Unit 3 moiety; and a drug moiety comprising Compound 1.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 13, HCDR2 comprising SEQ ID NO: 14, HCDR3 comprising SEQ ID NO: 15; LCDR1 comprising SEQ ID NO: 16, LCDR2 comprising SEQ ID NO: 17, and LCDR3 comprising SEQ ID NO: 18, as defined by the Kabat numbering system; and a linker-drug conjugate comprising LP20.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 19, HCDR2 comprising SEQ ID NO: 20, HCDR3 comprising SEQ ID NO: 21; LCDR1 comprising SEQ ID NO: 22, LCDR2 comprising SEQ ID NO: 23, and LCDR3 comprising SEQ ID NO: 24, as defined by the IMGT numbering system; a linker comprising MC-Val-Ala-pABC-Unit 3 moiety; and a drug moiety comprising Compound 1.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 19, HCDR2 comprising SEQ ID NO: 20, HCDR3 comprising SEQ ID NO: 21; LCDR1 comprising SEQ ID NO: 22, LCDR2 comprising SEQ ID NO: 23, and LCDR3 comprising SEQ ID NO: 24, as defined by the IMGT numbering system; and a linker-drug conjugate comprising LP20.

In some embodiments, the antibody-drug conjugate comprises an anti-CD73 antibody or antigen-binding fragment comprising a heavy chain variable region comprising an amino acid sequence of SEQ ID NO: 52, and a light chain variable region comprising an amino acid sequence of SEQ ID NO: 53; a linker comprising MC-Val-Ala-pABC-Unit 3 moiety; and a drug moiety comprising Compound 1.

In some embodiments, the antibody-drug conjugate comprises an anti-CD73 antibody or antigen-binding fragment comprising a heavy chain variable region comprising an amino acid sequence of SEQ ID NO: 52, and a light chain variable region comprising an amino acid sequence of SEQ ID NO: 53; and a linker-drug conjugate comprising LP20.

In some embodiments, the antibody-drug conjugate comprises an anti-CD73 antibody or antigen-binding fragment comprising a heavy chain variable region comprising an amino acid sequence of SEQ ID NO: 52, and a light chain variable region comprising an amino acid sequence of SEQ ID NO: 54; a linker comprising MC-Val-Ala-pABC-Unit 3 moiety; and a drug moiety comprising Compound 1.

In some embodiments, the antibody-drug conjugate comprises an anti-CD73 antibody or antigen-binding fragment comprising a heavy chain variable region comprising an amino acid sequence of SEQ ID NO: 52, and a light chain variable region comprising an amino acid sequence of SEQ ID NO: 54; and a linker-drug conjugate comprising LP20.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 13, HCDR2 comprising SEQ ID NO: 14, HCDR3 comprising SEQ ID NO: 15; LCDR1 comprising SEQ ID NO: 16, LCDR2 comprising SEQ ID NO: 17, and LCDR3 comprising SEQ ID NO: 18, as defined by the Kabat numbering system; a linker comprising Mal-Formula (II)-pABC-Unit 2 moiety; and a drug moiety comprising Compound 1.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 13, HCDR2 comprising SEQ ID NO: 14, HCDR3 comprising SEQ ID NO: 15; LCDR1 comprising SEQ ID NO: 16, LCDR2 comprising SEQ ID NO: 17, and LCDR3 comprising SEQ ID NO: 18, as defined by the Kabat numbering system; and a linker-drug conjugate comprising LP54.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 19, HCDR2 comprising SEQ ID NO: 20, HCDR3 comprising SEQ ID NO: 21; LCDR1 comprising SEQ ID NO: 22, LCDR2 comprising SEQ ID NO: 23, and LCDR3 comprising SEQ ID NO: 24, as defined by the IMGT numbering system; a linker comprising Mal-Formula (II)-Val-Ala-pABC-Unit 2 moiety; and a drug moiety comprising Compound 1.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 19, HCDR2 comprising SEQ ID NO: 20, HCDR3 comprising SEQ ID NO: 21; LCDR1 comprising SEQ ID NO: 22, LCDR2 comprising SEQ ID NO: 23, and LCDR3 comprising SEQ ID NO: 24, as defined by the IMGT numbering system; and a linker-drug conjugate comprising LP54.

In some embodiments, the antibody-drug conjugate comprises an anti-CD73 antibody or antigen-binding fragment comprising a heavy chain variable region comprising an amino acid sequence of SEQ ID NO: 52, and a light chain variable region comprising an amino acid sequence of SEQ ID NO: 53; a linker comprising Mal-Formula (II)-Val-Ala-pABC-Unit 2 moiety; and a drug moiety comprising Compound 1.

In some embodiments, the antibody-drug conjugate comprises an anti-CD73 antibody or antigen-binding fragment comprising a heavy chain variable region comprising an amino acid sequence of SEQ ID NO: 52, and a light chain variable region comprising an amino acid sequence of SEQ ID NO: 53; and a linker-drug conjugate comprising LP54.

In some embodiments, the antibody-drug conjugate comprises an anti-CD73 antibody or antigen-binding fragment comprising a heavy chain variable region comprising an amino acid sequence of SEQ ID NO: 52, and a light chain variable region comprising an amino acid sequence of SEQ ID NO: 54; a linker comprising Mal-Formula (II)-Val-Ala-pABC-Unit 2 moiety; and a drug moiety comprising Compound 1.

In some embodiments, the antibody-drug conjugate comprises an anti-CD73 antibody or antigen-binding fragment comprising a heavy chain variable region comprising an amino acid sequence of SEQ ID NO: 52, and a light chain variable region comprising an amino acid sequence of SEQ ID NO: 54; and a linker-drug conjugate comprising LP54.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 13, HCDR2 comprising SEQ ID NO: 14, HCDR3 comprising SEQ ID NO: 15; LCDR1 comprising SEQ ID NO: 16, LCDR2 comprising SEQ ID NO: 17, and LCDR3 comprising SEQ ID NO: 18, as defined by the Kabat numbering system; a linker comprising Mal-(PEG)₂-pABC-Unit 2 moiety; and a drug moiety comprising Compound 1.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 13, HCDR2 comprising SEQ ID NO: 14, HCDR3 comprising SEQ ID NO: 15; LCDR1 comprising SEQ ID NO: 16, LCDR2 comprising SEQ ID NO: 17, and LCDR3 comprising SEQ ID NO: 18, as defined by the Kabat numbering system; and a linker-drug conjugate comprising LP55.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 19, HCDR2 comprising SEQ ID NO: 20, HCDR3 comprising SEQ ID NO: 21; LCDR1 comprising SEQ ID NO: 22, LCDR2 comprising SEQ ID NO: 23, and LCDR3 comprising SEQ ID NO: 24, as defined by the IMGT numbering system; a linker comprising Mal-(PEG)₂-Val-Ala-pABC-Unit 2 moiety; and a drug moiety comprising Compound 1.

In some embodiments, the antibody-drug conjugate comprises an anti-CD73 antibody or antigen-binding fragment thereof comprising three heavy chain CDRs and three light chain CDRs as follows: HCDR1 comprising SEQ ID NO: 19, HCDR2 comprising SEQ ID NO: 20, HCDR3 comprising SEQ ID NO: 21; LCDR1 comprising SEQ ID NO: 22, LCDR2 comprising SEQ ID NO: 23, and LCDR3 comprising SEQ ID NO: 24, as defined by the IMGT numbering system; and a linker-drug conjugate comprising LP55.

In some embodiments, the antibody-drug conjugate comprises an anti-CD73 antibody or antigen-binding fragment comprising a heavy chain variable region comprising an amino acid sequence of SEQ ID NO: 52, and a light chain variable region comprising an amino acid sequence of SEQ ID NO: 53; a linker comprising Mal-(PEG)₂-Val-Ala-pABC-Unit 2 moiety; and a drug moiety comprising Compound 1.

In some embodiments, the antibody-drug conjugate comprises an anti-CD73 antibody or antigen-binding fragment comprising a heavy chain variable region comprising an amino acid sequence of SEQ ID NO: 52, and a light chain variable region comprising an amino acid sequence of SEQ ID NO: 53; and a linker-drug conjugate comprising LP55.

In some embodiments, the antibody-drug conjugate comprises an anti-CD73 antibody or antigen-binding fragment comprising a heavy chain variable region comprising an amino acid sequence of SEQ ID NO: 52, and a light chain variable region comprising an amino acid sequence of SEQ ID NO: 54; a linker comprising Mal-(PEG)₂-Val-Ala-pABC-Unit 2 moiety; and a drug moiety comprising Compound 1.

In some embodiments, the antibody-drug conjugate comprises an anti-CD73 antibody or antigen-binding fragment comprising a heavy chain variable region comprising an amino acid sequence of SEQ ID NO: 52, and a light chain variable region comprising an amino acid sequence of SEQ ID NO: 54; and a linker-drug conjugate comprising LP55.

In some embodiments of antibody-drug conjugates disclosed herein, the antibody-drug conjugate comprises an anti-CD73 antibody or antigen-binding fragment thereof which comprises three HCDRs comprising amino acid sequences of SEQ ID NO: 1 (HCDR1), SEQ ID NO: 2 (HCDR2), and SEQ ID NO: 3 (HCDR3); and three LCDRs comprising SEQ ID NO: 4 (LCDR1), SEQ ID NO: 5 (LCDR2), and SEQ ID NO: 6 (LCDR3), as defined by the Kabat numbering system; and a linker-drug conjugate comprising LP2, LP16, LP20, LP54, or LP55. Without being bound by theory, one or more of these antibody-drug conjugates may demonstrate superior properties over other ADCs, e.g., those using other antibodies, linkers, and/or drugs, e.g., as compared to other ADCs disclosed herein (e.g., improved conjugation stability, improved plasma stability, low ADC aggregation, on-target cytotoxicity, low off-target toxicity, pharmacokinetic and pharmacodynamics properties, formulatability, and toxicologic/immunologic profiles, stimulation of an anti-tumor immune response in the tumor microenvironment, stimulation of increased phagocytosis of CD73-expressing cells by myeloid cells (e.g., macrophages and/or dendritic cells), and in vivo anti-tumor activity). In some embodiments, without being bound by theory, benefits of using an antibody-drug conjugate comprising an anti-CD73 antibody or antigen-binding fragment thereof comprises three HCDRs comprising amino acid sequences of SEQ ID NO: 1 (HCDR1), SEQ ID NO: 2 (HCDR2), and SEQ ID NO: 3 (HCDR3); and three LCDRs comprising SEQ ID NO: 4 (LCDR1), SEQ ID NO: 5 (LCDR2), and SEQ ID NO: 6 (LCDR3), as defined by the Kabat numbering system; and a linker-drug conjugate comprising LP2, LP16, LP20, LP54, or LP55, may include improved conjugation stability, improved plasma stability, low ADC aggregation, on-target cytotoxicity, low off-target toxicity, pharmacokinetic and pharmacodynamics properties, formulatability, and toxicologic/immunologic profiles, stimulation of an anti-tumor immune response in the tumor microenvironment, stimulation of increased phagocytosis of CD73-expressing cells by myeloid cells (e.g., macrophages and/or dendritic cells), and in vivo anti-tumor activity. Exemplary evidence of the superior benefits of such antibody-drug conjugates is shown in Examples 4, 5, and 7.

In some embodiments of antibody-drug conjugates disclosed herein, the antibody-drug conjugate comprises an anti-CD73 antibody or antigen-binding fragment thereof which comprises three HCDRs comprising amino acid sequences of SEQ ID NO: 7 (HCDR1), SEQ ID NO: 8 (HCDR2), and SEQ ID NO: 9 (HCDR3); and three LCDRs comprising SEQ ID NO: 10 (LCDR1), SEQ ID NO: 11 (LCDR2), and SEQ ID NO: 12 (LCDR3), as defined by the IGMT numbering system; and a linker-drug conjugate comprising LP2, LP16, LP20, LP54, or LP55. Without being bound by theory, one or more of these antibody-drug conjugates may demonstrate superior properties over other ADCs, e.g., those using other antibodies, linkers, and/or drugs, e.g., as compared to other ADCs disclosed herein (e.g., improved conjugation stability, improved plasma stability, low ADC aggregation, on-target cytotoxicity, low off-target toxicity, pharmacokinetic and pharmacodynamics properties, formulatability, and toxicologic/immunologic profiles, stimulation of an anti-tumor immune response in the tumor microenvironment, stimulation of increased phagocytosis of CD73-expressing cells by myeloid cells (e.g., macrophages and/or dendritic cells), and in vivo anti-tumor activity). In some embodiments, without being bound by theory, benefits of using an antibody-drug conjugate comprising an anti-CD73 antibody or antigen-binding fragment thereof comprises three HCDRs comprising amino acid sequences of SEQ ID NO: 7 (HCDR1), SEQ ID NO: 8 (HCDR2), and SEQ ID NO: 9 (HCDR3); and three LCDRs comprising SEQ ID NO: 10 (LCDR1), SEQ ID NO: 11 (LCDR2), and SEQ ID NO: 12 (LCDR3), as defined by the IGMT numbering system; and a linker-drug conjugate comprising LP2, LP16, LP20, LP54, or LP55, may include improved conjugation stability, improved plasma stability, low ADC aggregation, on-target cytotoxicity, low off-target toxicity, pharmacokinetic and pharmacodynamics properties, formulatability, and toxicologic/immunologic profiles, stimulation of an anti-tumor immune response in the tumor microenvironment, stimulation of increased phagocytosis of CD73-expressing cells by myeloid cells (e.g., macrophages and/or dendritic cells), and in vivo anti-tumor activity. Exemplary evidence of the superior benefits of such antibody-drug conjugates is shown in Examples 4, 5, and 7.

In some embodiments of antibody-drug conjugates disclosed herein, the antibody-drug conjugate comprises an anti-CD73 antibody or antigen-binding fragment thereof which comprises a heavy chain variable region comprising an amino acid sequence of SEQ ID NO: 49, and a light chain variable region comprising an amino acid sequence of SEQ ID NO: 50; and a linker-drug conjugate comprising LP2, LP16, LP20, LP54, or LP55. Without being bound by theory, one or more of these antibody-drug conjugates may demonstrate superior properties over other ADCs, e.g., those using other antibodies, linkers, and/or drugs, e.g., as compared to other ADCs disclosed herein (e.g., improved conjugation stability, improved plasma stability, low ADC aggregation, on-target cytotoxicity, low off-target toxicity, pharmacokinetic and pharmacodynamics properties, formulatability, and toxicologic/immunologic profiles, stimulation of an anti-tumor immune response in the tumor microenvironment, stimulation of increased phagocytosis of CD73-expressing cells by myeloid cells (e.g., macrophages and/or dendritic cells), and in vivo anti-tumor activity). In some embodiments, without being bound by theory, benefits of using an antibody-drug conjugate comprising an anti-CD73 antibody or antigen-binding fragment thereof comprises which comprises a heavy chain variable region comprising an amino acid sequence of SEQ ID NO: 49, and a light chain variable region comprising an amino acid sequence of SEQ ID NO: 50; and a linker-drug conjugate comprising LP2, LP16, LP20, LP54, or LP55, may include improved conjugation stability, improved plasma stability, low ADC aggregation, on-target cytotoxicity, low off-target toxicity, pharmacokinetic and pharmacodynamics properties, formulatability, and toxicologic/immunologic profiles, stimulation of an anti-tumor immune response in the tumor microenvironment, stimulation of increased phagocytosis of CD73-expressing cells by myeloid cells (e.g., macrophages and/or dendritic cells), and in vivo anti-tumor activity. Exemplary evidence of the superior benefits of such antibody-drug conjugates is shown in Examples 4, 5, and 7.

In some embodiments of antibody-drug conjugates disclosed herein, the antibody-drug conjugate comprises an anti-CD73 antibody or antigen-binding fragment thereof which comprises a heavy chain variable region comprising an amino acid sequence of SEQ ID NO: 49, and a light chain variable region comprising an amino acid sequence of SEQ ID NO: 51; and a linker-drug conjugate comprising LP2, LP16, LP20, LP54, or LP55. Without being bound by theory, one or more of these antibody-drug conjugates may demonstrate superior properties over other ADCs, e.g., those using other antibodies, linkers, and/or drugs, e.g., as compared to other ADCs disclosed herein (e.g., improved conjugation stability, improved plasma stability, low ADC aggregation, on-target cytotoxicity, low off-target toxicity, pharmacokinetic and pharmacodynamics properties, formulatability, and toxicologic/immunologic profiles, stimulation of an anti-tumor immune response in the tumor microenvironment, stimulation of increased phagocytosis of CD73-expressing cells by myeloid cells (e.g., macrophages and/or dendritic cells), and in vivo anti-tumor activity). In some embodiments, without being bound by theory, benefits of using an antibody-drug conjugate comprising an anti-CD73 antibody or antigen-binding fragment thereof comprises which comprises a heavy chain variable region comprising an amino acid sequence of SEQ ID NO: 49, and a light chain variable region comprising an amino acid sequence of SEQ ID NO: 51; and a linker-drug conjugate comprising LP2, LP16, LP20, LP54, or LP55, may include improved conjugation stability, improved plasma stability, low ADC aggregation, on-target cytotoxicity, low off-target toxicity, pharmacokinetic and pharmacodynamics properties, formulatability, and toxicologic/immunologic profiles, stimulation of an anti-tumor immune response in the tumor microenvironment, stimulation of increased phagocytosis of CD73-expressing cells by myeloid cells (e.g., macrophages and/or dendritic cells), and in vivo anti-tumor activity. Exemplary evidence of the superior benefits of such antibody-drug conjugates is shown in Examples 4, 5, and 7.

In some embodiments of antibody-drug conjugates disclosed herein, the antibody-drug conjugate comprises an anti-CD73 antibody or antigen-binding fragment thereof which comprises three HCDRs comprising amino acid sequences of SEQ ID NO: 13 (HCDR1), SEQ ID NO: 14 (HCDR2), and SEQ ID NO: 15 (HCDR3); and three LCDRs comprising SEQ ID NO: 16 (LCDR1), SEQ ID NO: 17 (LCDR2), and SEQ ID NO: 18 (LCDR3), as defined by the Kabat numbering system; and a linker-drug conjugate comprising LP2, LP16, LP20, LP54, or LP55. Without being bound by theory, one or more of these antibody-drug conjugates may demonstrate superior properties over other ADCs, e.g., those using other antibodies, linkers, and/or drugs, e.g., as compared to other ADCs disclosed herein (e.g., improved conjugation stability, improved plasma stability, low ADC aggregation, on-target cytotoxicity, low off-target toxicity, pharmacokinetic and pharmacodynamics properties, formulatability, and toxicologic/immunologic profiles, stimulation of an anti-tumor immune response in the tumor microenvironment, stimulation of increased phagocytosis of CD73-expressing cells by myeloid cells (e.g., macrophages and/or dendritic cells), and in vivo anti-tumor activity). In some embodiments, without being bound by theory, benefits of using an antibody-drug conjugate comprising an anti-CD73 antibody or antigen-binding fragment thereof comprises three HCDRs comprising amino acid sequences of SEQ ID NO: 13 (HCDR1), SEQ ID NO: 14 (HCDR2), and SEQ ID NO: 15 (HCDR3); and three LCDRs comprising SEQ ID NO: 16 (LCDR1), SEQ ID NO: 17 (LCDR2), and SEQ ID NO: 18 (LCDR3), as defined by the Kabat numbering system; and a linker-drug conjugate comprising LP2, LP16, LP20, LP54, or LP55, may include improved conjugation stability, improved plasma stability, low ADC aggregation, on-target cytotoxicity, low off-target toxicity, pharmacokinetic and pharmacodynamics properties, formulatability, and toxicologic/immunologic profiles, stimulation of an anti-tumor immune response in the tumor microenvironment, stimulation of increased phagocytosis of CD73-expressing cells by myeloid cells (e.g., macrophages and/or dendritic cells), and in vivo anti-tumor activity. Exemplary evidence of the superior benefits of such antibody-drug conjugates is shown in Examples 4, 5, and 7.

In some embodiments of antibody-drug conjugates disclosed herein, the antibody-drug conjugate comprises an anti-CD73 antibody or antigen-binding fragment thereof which comprises three HCDRs comprising amino acid sequences of SEQ ID NO: 19 (HCDR1), SEQ ID NO: 20 (HCDR2), and SEQ ID NO: 21 (HCDR3); and three LCDRs comprising SEQ ID NO: 22 (LCDR1), SEQ ID NO: 23 (LCDR2), and SEQ ID NO: 24 (LCDR3), as defined by the IGMT numbering system; and a linker-drug conjugate comprising LP2, LP16, LP20, LP54, or LP55. Without being bound by theory, one or more of these antibody-drug conjugates may demonstrate superior properties over other ADCs, e.g., those using other antibodies, linkers, and/or drugs, e.g., as compared to other ADCs disclosed herein (e.g., improved conjugation stability, improved plasma stability, low ADC aggregation, on-target cytotoxicity, low off-target toxicity, pharmacokinetic and pharmacodynamics properties, formulatability, and toxicologic/immunologic profiles, stimulation of an anti-tumor immune response in the tumor microenvironment, stimulation of increased phagocytosis of CD73-expressing cells by myeloid cells (e.g., macrophages and/or dendritic cells), and in vivo anti-tumor activity). In some embodiments, without being bound by theory, benefits of using an antibody-drug conjugate comprising an anti-CD73 antibody or antigen-binding fragment thereof comprises three HCDRs comprising amino acid sequences of SEQ ID NO: 19 (HCDR1), SEQ ID NO: 20 (HCDR2), and SEQ ID NO: 21 (HCDR3); and three LCDRs comprising SEQ ID NO: 22 (LCDR1), SEQ ID NO: 23 (LCDR2), and SEQ ID NO: 24 (LCDR3), as defined by the IGMT numbering system; and a linker-drug conjugate comprising LP2, LP16, LP20, LP54, or LP55, may include improved conjugation stability, improved plasma stability, low ADC aggregation, on-target cytotoxicity, low off-target toxicity, pharmacokinetic and pharmacodynamics properties, formulatability, and toxicologic/immunologic profiles, stimulation of an anti-tumor immune response in the tumor microenvironment, stimulation of increased phagocytosis of CD73-expressing cells by myeloid cells (e.g., macrophages and/or dendritic cells), and in vivo anti-tumor activity. Exemplary evidence of the superior benefits of such antibody-drug conjugates is shown in Examples 4, 5, and 7.

In some embodiments of antibody-drug conjugates disclosed herein, the antibody-drug conjugate comprises an anti-CD73 antibody or antigen-binding fragment thereof which comprises a heavy chain variable region comprising an amino acid sequence of SEQ ID NO: 52, and a light chain variable region comprising an amino acid sequence of SEQ ID NO: 53; and a linker-drug conjugate comprising LP2, LP16, LP20, LP54, or LP55. Without being bound by theory, one or more of these antibody-drug conjugates may demonstrate superior properties over other ADCs, e.g., those using other antibodies, linkers, and/or drugs, e.g., as compared to other ADCs disclosed herein (e.g., improved conjugation stability, improved plasma stability, low ADC aggregation, on-target cytotoxicity, low off-target toxicity, pharmacokinetic and pharmacodynamics properties, formulatability, and toxicologic/immunologic profiles, stimulation of an anti-tumor immune response in the tumor microenvironment, stimulation of increased phagocytosis of CD73-expressing cells by myeloid cells (e.g., macrophages and/or dendritic cells), and in vivo anti-tumor activity). In some embodiments, without being bound by theory, benefits of using an antibody-drug conjugate comprising an anti-CD73 antibody or antigen-binding fragment thereof comprises which comprises a heavy chain variable region comprising an amino acid sequence of SEQ ID NO: 52, and a light chain variable region comprising an amino acid sequence of SEQ ID NO: 53; and a linker-drug conjugate comprising LP2, LP16, LP20, LP54, or LP55, may include improved conjugation stability, improved plasma stability, low ADC aggregation, on-target cytotoxicity, low off-target toxicity, pharmacokinetic and pharmacodynamics properties, formulatability, and toxicologic/immunologic profiles, stimulation of an anti-tumor immune response in the tumor microenvironment, stimulation of increased phagocytosis of CD73-expressing cells by myeloid cells (e.g., macrophages and/or dendritic cells), and in vivo anti-tumor activity. Exemplary evidence of the superior benefits of such antibody-drug conjugates is shown in Examples 4, 5, and 7.

In some embodiments of antibody-drug conjugates disclosed herein, the antibody-drug conjugate comprises an anti-CD73 antibody or antigen-binding fragment thereof which comprises a heavy chain variable region comprising an amino acid sequence of SEQ ID NO: 52, and a light chain variable region comprising an amino acid sequence of SEQ ID NO: 54; and a linker-drug conjugate comprising LP2, LP16, LP20, LP54, or LP55. Without being bound by theory, one or more of these antibody-drug conjugates may demonstrate superior properties over other ADCs, e.g., those using other antibodies, linkers, and/or drugs, e.g., as compared to other ADCs disclosed herein (e.g., improved conjugation stability, improved plasma stability, low ADC aggregation, on-target cytotoxicity, low off-target toxicity, pharmacokinetic and pharmacodynamics properties, formulatability, and toxicologic/immunologic profiles, stimulation of an anti-tumor immune response in the tumor microenvironment, stimulation of increased phagocytosis of CD73-expressing cells by myeloid cells (e.g., macrophages and/or dendritic cells), and in vivo anti-tumor activity). In some embodiments, without being bound by theory, benefits of using an antibody-drug conjugate comprising an anti-CD73 antibody or antigen-binding fragment thereof comprises which comprises a heavy chain variable region comprising an amino acid sequence of SEQ ID NO: 52, and a light chain variable region comprising an amino acid sequence of SEQ ID NO: 54; and a linker-drug conjugate comprising LP2, LP16, LP20, LP54, or LP55 may include improved conjugation stability, improved plasma stability, low ADC aggregation, on-target cytotoxicity, low off-target toxicity, pharmacokinetic and pharmacodynamics properties, formulatability, and toxicologic/immunologic profiles, stimulation of an anti-tumor immune response in the tumor microenvironment, stimulation of increased phagocytosis of CD73-expressing cells by myeloid cells (e.g., macrophages and/or dendritic cells), and in vivo anti-tumor activity. Exemplary evidence of the superior benefits of such antibody-drug conjugates is shown in Examples 4, 5, and 7.
1. An internalizing anti-CD73 antibody or internalizing antigen-binding fragment thereof, wherein the antibody or antigen-binding fragment binds specifically to human CD73, and wherein the antibody or antigen-binding fragment comprises three heavy chain complementarity determining regions (HCDR1, HCDR2, and HCDR3) and three light chain complementarity determining regions (LCDR1, LCDR2, and LCDR3), wherein
(i) the HCDR1 comprises an amino acid sequence of SEQ ID NO: 1, the HCDR2 comprises an amino acid sequence of SEQ ID NO: 2, the HCDR3 comprises an amino acid sequence of SEQ ID NO: 3, the LCDR1 comprises an amino acid sequence of SEQ ID NO: 4, the LCDR2 comprises an amino acid sequence of SEQ ID NO: 5, and the LCDR3 comprises an amino acid sequence of SEQ ID NO: 6, as defined by the Kabat numbering system;
(ii) the HCDR1 comprises an amino acid sequence of SEQ ID NO: 7, the HCDR2 comprises an amino acid sequence of SEQ ID NO: 8, the HCDR3 comprises an amino acid sequence of SEQ ID NO: 9, the LCDR1 comprises an amino acid sequence of SEQ ID NO: 10, the LCDR2 comprises an amino acid sequence of SEQ ID NO: 11, and the LCDR3 comprises an amino acid sequence of SEQ ID NO: 12, as defined by the IMGT numbering system;
(iii) the HCDR1 comprises an amino acid sequence of SEQ ID NO: 13, the HCDR2 comprises an amino acid sequence of SEQ ID NO: 14, the HCDR3 comprises an amino acid sequence of SEQ ID NO: 15, the LCDR1 comprises an amino acid sequence of SEQ ID NO: 16, the LCDR2 comprises an amino acid sequence of SEQ ID NO: 17, and the LCDR3 comprises an amino acid sequence of SEQ ID NO: 18, as defined by the Kabat numbering system;
(iv) the HCDR1 comprises an amino acid sequence of SEQ ID NO: 19, the HCDR2 comprises an amino acid sequence of SEQ ID NO: 20, the HCDR3 comprises an amino acid sequence of SEQ ID NO: 21, the LCDR1 comprises an amino acid sequence of SEQ ID NO: 22, the LCDR2 comprises an amino acid sequence of SEQ ID NO: 23, and the LCDR3 comprises an amino acid sequence of SEQ ID NO: 24, as defined by the IMGT numbering system;
(v) the HCDR1 comprises an amino acid sequence of SEQ ID NO: 25, the HCDR2 comprises an amino acid sequence of SEQ ID NO: 26, the HCDR3 comprises an amino acid sequence of SEQ ID NO: 27, the LCDR1 comprises an amino acid sequence of SEQ ID NO: 28, the LCDR2 comprises an amino acid sequence of SEQ ID NO: 29, and the LCDR3 comprises an amino acid sequence of SEQ ID NO: 30, as defined by the Kabat numbering system;
(vi) the HCDR1 comprises an amino acid sequence of SEQ ID NO: 31, the HCDR2 comprises an amino acid sequence of SEQ ID NO: 32, the HCDR3 comprises an amino acid sequence of SEQ ID NO: 33, the LCDR1 comprises an amino acid sequence of SEQ ID NO: 34, the LCDR2 comprises an amino acid sequence of SEQ ID NO: 35, and the LCDR3 comprises an amino acid sequence of SEQ ID NO: 36, as defined by the IMGT numbering system;
(vii) the HCDR1 comprises an amino acid sequence of SEQ ID NO: 37, the HCDR2 comprises an amino acid sequence of SEQ ID NO: 38, the HCDR3 comprises an amino acid sequence of SEQ ID NO: 39, the LCDR1 comprises an amino acid sequence of SEQ ID NO: 40, the LCDR2 comprises an amino acid sequence of SEQ ID NO: 41, and the LCDR3 comprises an amino acid sequence of SEQ ID NO: 42, as defined by the Kabat numbering system; or
(viii) the HCDR1 comprises an amino acid sequence of SEQ ID NO: 43, the HCDR2 comprises an amino acid sequence of SEQ ID NO: 44, the HCDR3 comprises an amino acid sequence of SEQ ID NO: 45, the LCDR1 comprises an amino acid sequence of SEQ ID NO: 46, the LCDR2 comprises an amino acid sequence of SEQ ID NO: 47, and the LCDR3 comprises an amino acid sequence of SEQ ID NO: 48, as defined by the IMGT numbering system.
2. The anti-CD73 antibody or antigen-binding fragment of embodiment 1, wherein the antibody or antigen-binding fragment comprises
(i) three HCDRs comprising amino acid sequences of SEQ ID NO: 1 (HCDR1), SEQ ID NO: 2 (HCDR2), and SEQ ID NO: 3 (HCDR3); and three LCDRs comprising amino acid sequences of SEQ ID NO: 4 (LCDR1), SEQ ID NO: 5 (LCDR2), and SEQ ID NO: 6 (LCDR3), as defined by the Kabat numbering system; or
(ii) three HCDRs comprising amino acid sequences of SEQ ID NO: 7 (HCDR1), SEQ ID NO: 8 (HCDR2), and SEQ ID NO: 9 (HCDR3); and three LCDRs comprising amino acid sequences of SEQ ID NO: 10 (LCDR1), SEQ ID NO: 11 (LCDR2), and SEQ ID NO: 12 (LCDR3), as defined by the IMGT numbering system.
3. The anti-CD73 antibody or antigen-binding fragment of embodiment 1, wherein the antibody or antigen-binding fragment comprises
(i) three HCDRs comprising amino acid sequences of SEQ ID NO: 13 (HCDR1), SEQ ID NO: 14 (HCDR2), and SEQ ID NO: 15 (HCDR3); and three LCDRs comprising amino acid sequences of SEQ ID NO: 16 (LCDR1), SEQ ID NO: 17 (LCDR2), and SEQ ID NO: 18 (LCDR3), as defined by the Kabat numbering system; or
(ii) three HCDRs comprising amino acid sequences of SEQ ID NO: 19 (HCDR1), SEQ ID NO: 20 (HCDR2), and SEQ ID NO: 21 (HCDR3); and three LCDRs comprising amino acid sequences of SEQ ID NO: 22 (LCDR1), SEQ ID NO: 23 (LCDR2), and SEQ ID NO: 24 (LCDR3), as defined by the IMGT numbering system.
4. The anti-CD73 antibody or antigen-binding fragment of any one of embodiments 1 to 3, wherein the antibody or antigen-binding fragment comprises
(i) a heavy chain variable region comprising an amino acid sequence of SEQ ID NO: 49, and a light chain variable region comprising an amino acid sequence selected from SEQ ID NO: 50 or 51;
(ii) a heavy chain variable region comprising an amino acid sequence of SEQ ID NO: 52, and a light chain variable region comprising an amino acid sequence selected from SEQ ID NO: 53 or 54;
(iii) a heavy chain variable region comprising an amino acid sequence of SEQ ID NO: 55, and a light chain variable region comprising an amino acid sequence selected from SEQ ID NO: 56 or 57; or
(iv) a heavy chain variable region comprising an amino acid sequence of SEQ ID NO: 58, and a light chain variable region comprising an amino acid sequence selected from SEQ ID NO: 59 or 60.
5. The anti-CD73 antibody or antigen-binding fragment of embodiment 4, wherein the antibody or antigen-binding fragment comprises
(i) a heavy chain variable region comprising an amino acid sequence of SEQ ID NO: 49, and a light chain variable region comprising an amino acid sequence of SEQ ID NO: 50; or
(ii) a heavy chain variable region comprising an amino acid sequence of SEQ ID NO: 49, and a light chain variable region comprising an amino acid sequence of SEQ ID NO: 51.
6. The anti-CD73 antibody or antigen-binding fragment of embodiment 4, wherein the antibody or antigen-binding fragment comprises
(i) a heavy chain variable region comprising an amino acid sequence of SEQ ID NO: 52, and a light chain variable region comprising an amino acid sequence of SEQ ID NO: 53; or
(ii) a heavy chain variable region comprising an amino acid sequence of SEQ ID NO: 52, and a light chain variable region comprising an amino acid sequence of SEQ ID NO: 54.
7. The anti-CD73 antibody or antigen-binding fragment of any one of embodiments 1 to 6, wherein the antibody or antigen-binding fragment comprises a human IgG heavy chain constant region.
8. The anti-CD73 antibody or antigen-binding fragment of any one of embodiments 1 to 7, wherein the antibody or antigen-binding fragment comprises a human IgG1, IgG2, or IgG4 heavy chain constant region.
9. The anti-CD73 antibody or antigen-binding fragment of embodiment 8, wherein the antibody or antigen-binding fragment comprises an IgG1 heavy chain constant region comprising an Fc domain mutated to reduce binding to an Fcγ receptor (FcγR) as compared to an IgG1 heavy chain constant region comprising a wild type IgG1 Fc domain.
10. The anti-CD73 antibody or antigen-binding fragment of embodiment 9, wherein the mutated IgG1 Fc domain comprises the mutations L234A, L235A, P238S, H268Q, and K274Q.
11. The anti-CD73 antibody or antigen-binding fragment of any one of embodiments 1 to 10, wherein the antibody or antigen-binding fragment comprises a human Ig kappa light chain constant region.
12. The anti-CD73 antibody or antigen-binding fragment of any one of embodiments 1 to 11, wherein the antibody or antigen-binding fragment comprises:
(i) a heavy chain comprising an amino acid sequence selected from SEQ ID NO: 61, 62, 63, or 64 and a light chain comprising an amino acid sequence selected from SEQ ID NO: 65 or 66;
(ii) a heavy chain comprising an amino acid sequence selected from SEQ ID NO: 67, 68, 69, or 70 and a light chain comprising an amino acid sequence selected from SEQ ID NO: 71 or 72;
(iii) a heavy chain comprising an amino acid sequence selected from SEQ ID NO: 73, 74, 75, or 76 and a light chain comprising an amino acid sequence selected from SEQ ID NO: 77 or 78; or
(iv) a heavy chain comprising an amino acid sequence selected from SEQ ID NO: 79, 80, 81, or 82 and a light chain comprising an amino acid sequence selected from SEQ ID NO: 83 or 84.
13. The anti-CD73 antibody or antigen-binding fragment of embodiment 12, wherein the antibody or antigen-binding fragment comprises:
(i) a heavy chain comprising an amino acid sequence of SEQ ID NO: 63 and a light chain comprising an amino acid sequence of SEQ ID NO: 65;
(ii) a heavy chain comprising an amino acid sequence of SEQ ID NO: 63 and a light chain comprising an amino acid sequence of SEQ ID NO: 66;
(iii) a heavy chain comprising an amino acid sequence of SEQ ID NO: 64 and a light chain comprising an amino acid sequence of SEQ ID NO: 65;
(iv) a heavy chain comprising an amino acid sequence of SEQ ID NO: 64 and a light chain comprising an amino acid sequence of SEQ ID NO: 66;
(v) a heavy chain comprising an amino acid sequence of SEQ ID NO: 69 and a light chain comprising an amino acid sequence of SEQ ID NO: 71;
(vi) a heavy chain comprising an amino acid sequence of SEQ ID NO: 69 and a light chain comprising an amino acid sequence of SEQ ID NO: 72;
(vii) a heavy chain comprising an amino acid sequence of SEQ ID NO: 70 and a light chain comprising an amino acid sequence of SEQ ID NO: 71; or
(viii) a heavy chain comprising an amino acid sequence of SEQ ID NO: 70 and a light chain comprising an amino acid sequence of SEQ ID NO: 72.
14. The anti-CD73 antibody or antigen-binding fragment of any one of embodiments 1 to 11, wherein the antibody or antigen-binding fragment comprises:
(i) a heavy chain comprising an amino acid sequence selected from SEQ ID NO: 111, 112, 113, or 114 and a light chain comprising an amino acid sequence selected from SEQ ID NO: 65 or 66;
(ii) a heavy chain comprising an amino acid sequence selected from SEQ ID NO: 115, 116, 117, or 118 and a light chain comprising an amino acid sequence selected from SEQ ID NO: 71 or 72;
(iii) a heavy chain comprising an amino acid sequence selected from SEQ ID NO: 119, 120, 121, or 122 and a light chain comprising an amino acid sequence selected from SEQ ID NO: 77 or 78; or
(iv) a heavy chain comprising an amino acid sequence selected from SEQ ID NO: 123, 124, 125, or 126 and a light chain comprising an amino acid sequence selected from SEQ ID NO: 83 or 84.
15. The anti-CD73 antibody or antigen-binding fragment of any one of embodiments 1 to 14, wherein the antibody or antigen-binding fragment comprises part of a bispecific or multi-specific binding construct.
16. The anti-CD73 antibody or antigen-binding fragment of any one of embodiments 1 to 15, wherein the antibody or antigen-binding fragment is linked to a therapeutic agent or detectable agent.
17. An antibody-drug conjugate of Formula (I):

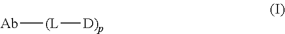

wherein Ab is an anti-CD73 antibody or antigen-binding fragment thereof of any one of embodiments 1 to 14;
D is a therapeutic agent;
L is a cleavable linker that covalently attaches Ab to D; and
p is an integer from 1 to 20.
18. The antibody-drug conjugate of embodiment 17, wherein the therapeutic agent comprises a STING agonist.
19. The antibody-drug conjugate of embodiment 18, wherein the therapeutic agent comprises a compound selected from:

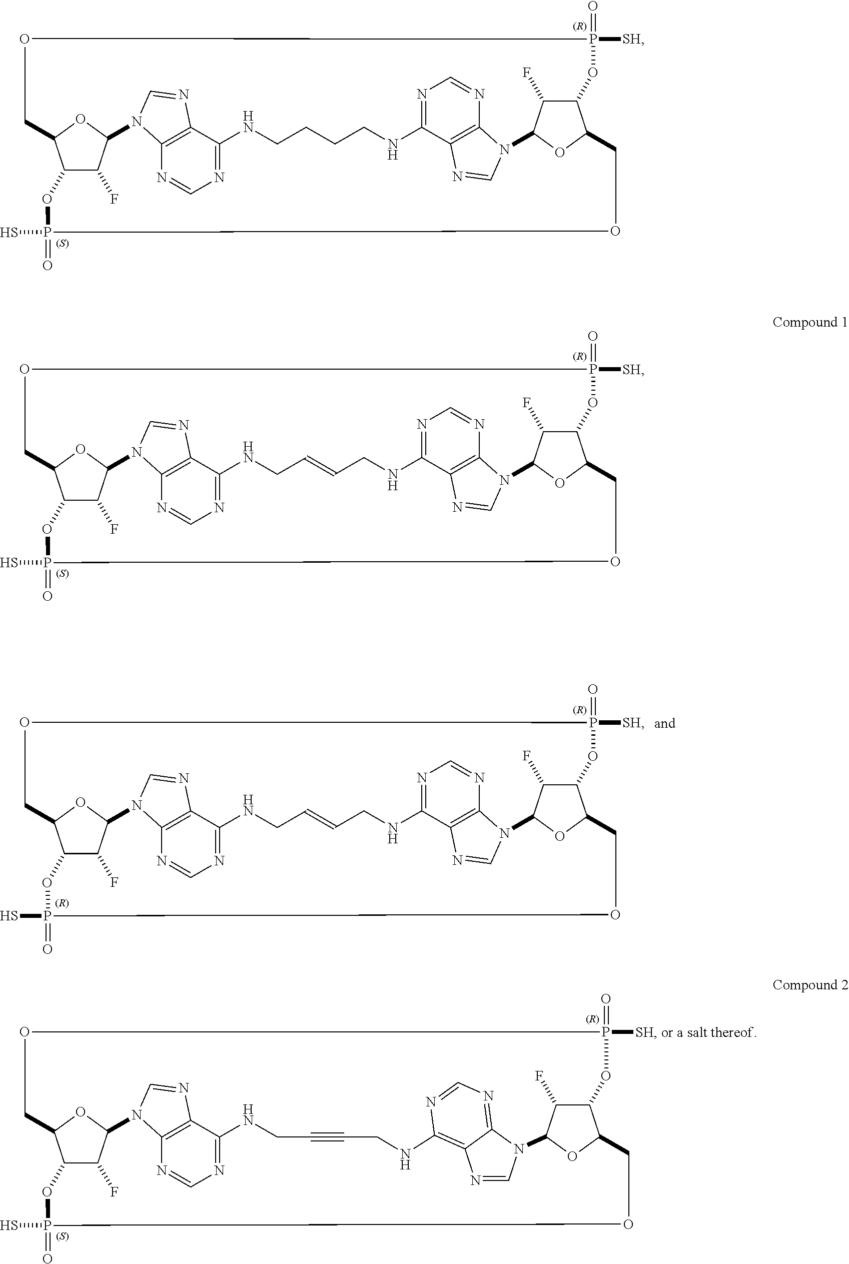

20. The antibody-drug conjugate of embodiment 19, wherein D comprises Compound 1.
21. The antibody-drug conjugate of embodiment 19, wherein D comprises Compound 2.
22. The antibody-drug conjugate of any one of embodiments 17 to 21, wherein p is from 2 to 8.
23. The antibody-drug conjugate of embodiment 22, wherein p is 2 or 4.
24. The antibody-drug conjugate of any one of embodiments 17 to 23, wherein the cleavable linker comprises a cleavable moiety that is positioned such that no part of the linker or the antibody or antigen-binding fragment remains bound to the cytotoxic agent upon cleavage.
25. The antibody-drug conjugate of any one of embodiments 17 to 24, wherein the cleavable linker comprises a cleavable peptide moiety.
26. The antibody-drug conjugate of embodiment 25, wherein the cleavable peptide moiety is cleavable by a protease, optionally wherein the protease is a cathepsin or a legumain.
27. The antibody-drug conjugate of embodiment 25 or embodiment 26, wherein the cleavable peptide moiety comprises an amino acid unit.
28. The antibody-drug conjugate of embodiment 27, wherein the amino acid unit comprises valine-alanine (Val-Ala), valine-citrulline (Val-Cit), valine-lysine (Val-Lys), alanine-alanine-asparagine (Ala-Ala-Asn), Ala-(NMe)Ala-Asn, glycine-glycine-phenylalanine-glycine (Gly-Gly-Phe-Gly) (SEQ ID NO: 244), valine-dimethylated lysine (Val-Lys(Me)₂), asparagine (Asn), aspartic acid (Asp), methylated aspartic acid (Asp(OMe)), glutamic acid-valine-alanine (Glu-Val-Ala), or glycine-valine-alanine (Gly-Val-Ala).
29. The antibody-drug conjugate of embodiment 28, wherein the amino acid unit comprises Val-Ala.
30. The antibody-drug conjugate of embodiment 28, wherein the amino acid unit comprises Val-Cit.
31. The antibody-drug conjugate of embodiment 28, wherein the amino acid unit comprises Gly-Gly-Phe-Gly (SEQ ID NO: 244).
32. The antibody-drug conjugate of any one of embodiments 17 to 31, wherein the cleavable linker attaches to the antibody or antigen-binding fragment via a maleimide (Mal) moiety.
33. The antibody-drug conjugate of embodiment 32, wherein the Mal moiety comprises a maleimidocaproyl (MC) moiety.
34. The antibody-drug conjugate of embodiment 32, wherein the Mal moiety comprises a dithiomaleimide (DTM) moiety.
35. The antibody-drug conjugate of any one of embodiments 32 to 34, wherein the Mal moiety is reactive with a cysteine residue on the antibody or antigen-binding fragment.
36. The antibody-drug conjugate of any one of embodiments 32 to 35, wherein the Mal moiety is joined to the antibody or antigen-binding fragment via a cysteine residue on the antibody or antigen-binding fragment.
37. The antibody-drug conjugate of any one of embodiments 17 to 36, wherein the cleavable linker comprises a Mal moiety and a cleavable peptide moiety.
38. The antibody-drug conjugate of embodiment 37, wherein the Mal moiety attaches the antibody or antigen-binding fragment to the cleavable peptide moiety in the cleavable linker.
39. The antibody drug conjugate of any one of embodiments 33 and 35 to 38, wherein the cleavable linker comprises MC-Val-Ala.
40. The antibody drug conjugate of any one of embodiments 33 and 35 to 38, wherein the cleavable linker comprises MC-Val-Cit.
41. The antibody drug conjugate of any one of embodiments 33 and 35 to 38, wherein the cleavable linker comprises MC-Gly-Gly-Phe-Gly (SEQ ID NO: 249).
42. The antibody-drug conjugate of any one of embodiments 17 to 41, wherein the cleavable linker comprises at least one spacer unit.
43. The antibody-drug conjugate of embodiment 42, wherein the spacer unit comprises a polyethylene glycol (PEG) moiety.
44. The antibody-drug conjugate of embodiment 43, wherein the PEG moiety comprises -(PEG)ₘ- and m is an integer from 1 to 10.
45. The antibody-drug conjugate of embodiment 44, wherein m is 2 to 8.
46. The antibody-drug conjugate of embodiment 44 or embodiment 45, wherein m is 2 to 5.
47. The antibody-drug conjugate of any one of embodiments 44 to 46, wherein m is 2.
48. The antibody-drug conjugate of any one of embodiments 17 to 47, wherein the cleavable linker comprises at least one self-immolative unit.
49. The antibody-drug conjugate of embodiment 48, wherein the cleavable linker comprises a first self-immolative unit.
50. The antibody-drug conjugate of embodiment 49, wherein the cleavable linker is capable of being removed from D after cleavage of the linker by self-immolation of the first self-immolative unit.
51. The antibody-drug conjugate of embodiment 49 or embodiment 50, wherein the first self-immolative unit comprises a p-aminobenzyl (pAB).
52. The antibody-drug conjugate of any one of embodiments 48 to 51, wherein the cleavable linker comprises MC-Val-Ala-pAB.
53. The antibody-drug conjugate of any one of embodiments 48 to 51, wherein the cleavable linker comprises MC-Val-Cit-pAB.
54. The antibody-drug conjugate of any one of embodiments 48 to 51, wherein the cleavable linker comprises MC-Gly-Gly-Phe-Gly-pAB (SEQ ID NO: 250).
55. The antibody-drug conjugate of embodiment 49 or embodiment 50, wherein the first self-immolative unit comprises a p-aminobenzyloxycarbonyl (pABC).
56. The antibody-drug conjugate of any one of embodiments 48 to 50 and 55, wherein the cleavable linker comprises MC-Val-Ala-pABC.
57. The antibody-drug conjugate of any one of embodiments 48 to 50 and 55, wherein the cleavable linker comprises MC-Val-Cit-pABC.
58. The antibody-drug conjugate of any one of embodiments 48 to 50 and 55, wherein the cleavable linker comprises MC-Gly-Gly-Phe-Gly-pABC (SEQ ID NO: 251).
59. The antibody-drug conjugate of any one of embodiments 48 to 58, wherein the cleavable linker further comprises a second self-immolative unit.
60. The antibody-drug conjugate of embodiment 59, wherein the cleavable linker is capable of being removed from D after cleavage of the linker by self-immolation of the first self-immolative unit and/or self-immolation of the second self-immolative unit.
61. The antibody-drug conjugate of embodiment 59 or embodiment 60, wherein the cleavable linker is removed from D after cleavage of the linker in a stepwise fashion by self-immolation of the first self-immolative unit and then self-immolation of the second self-immolative unit.
62. The antibody-drug conjugate of any one of embodiments 17 to 61, wherein the cleavable linker comprises a cleavable peptide moiety, a first self-immolative unit, and a second self-immolative unit.
63. The antibody-drug conjugate of embodiment 62, wherein the cleavable peptide moiety comprises Val-Ala.
64. The antibody-drug conjugate of embodiment 62, wherein the cleavable peptide moiety comprises Val-Cit.
65. The antibody-drug conjugate of embodiment 62, wherein the cleavable peptide moiety comprises Gly-Phe-Phe-Gly (SEQ ID NO: 253).
66. The antibody-drug conjugate of embodiment 62, wherein the second self-immolative unit comprises one of the following moieties:

| Second Self- | |
|---|---|
| immolative Unit | Chemical Structure |
| Unit 1 (MEC) | 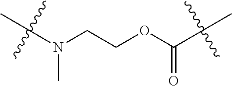 [SMILES: *C(=O)OCCN(*)C] |
| Unit 2 | 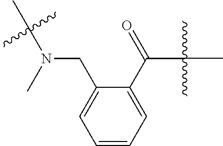 [SMILES: *C(=O)c1ccccc1CN(*)C] |
| Unit 3 | 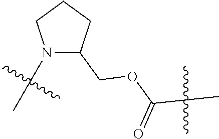 [SMILES: *C(=O)OCC1CCCN1*] |

or an isomer thereof.
67. The antibody-drug conjugate of embodiment 62, wherein the cleavable linker comprises Val-Ala and wherein the second self-immolative unit comprises one of the following moieties:

| Second Self- | |
|---|---|
| immolative Unit | Chemical Structure |
| Unit 1 (MEC) | 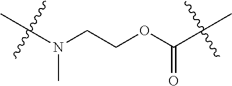 [SMILES: *C(=O)OCCN(*)C] |
| Unit 2 | 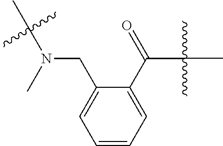 [SMILES: *C(=O)c1ccccc1CN(*)C] |
| Unit 3 | 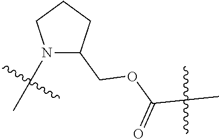 [SMILES: *C(=O)OCC1CCCN1*] |

or an isomer thereof.
68. The antibody-drug conjugate of any one of embodiments 62 to 67, wherein the second self-immolative unit comprises a Unit 1 (MEC) moiety.
69. The antibody-drug conjugate of any one of embodiments 62 to 67, wherein the second self-immolative unit comprises a Unit 2 moiety.
70. The antibody-drug conjugate of any one of embodiments 62 to 67, wherein the second self-immolative unit comprises a Unit 3 moiety.
71. The antibody-drug conjugate of embodiment 67, wherein the linker comprises Val-Ala-pABC-Unit 1 moiety.
72. The antibody-drug conjugate of embodiment 67, wherein the linker comprises MC-Val-Ala-pABC-Unit 1 moiety.
73. The antibody-drug conjugate of embodiment 17, wherein the L-D comprises LP2:

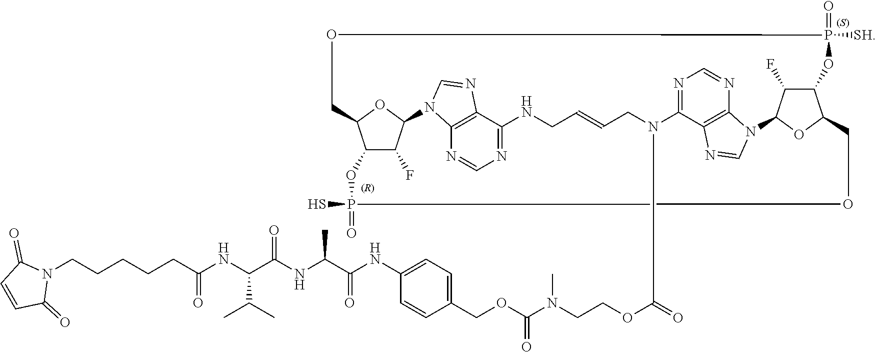

74. The antibody-drug conjugate of embodiment 66, wherein the linker comprises Val-Cit-pABC-Unit 1 moiety.
75. The antibody-drug conjugate of embodiment 66, wherein the linker comprises MC-Val-Cit-pABC-Unit 1 moiety.
76. The antibody-drug conjugate of embodiment 66, wherein the L-D comprises MC-Val-Cit-pABC-Unit 1-Compound 1.
77. The antibody-drug conjugate of embodiment 67, wherein the linker comprises Val-Ala-pABC-Unit 2 moiety.
78. The antibody-drug conjugate of embodiment 67, wherein the linker comprises MC-Val-Ala-pABC-Unit 2 moiety.
79. The antibody-drug conjugate of embodiment 17, wherein the L-D comprises LP16:

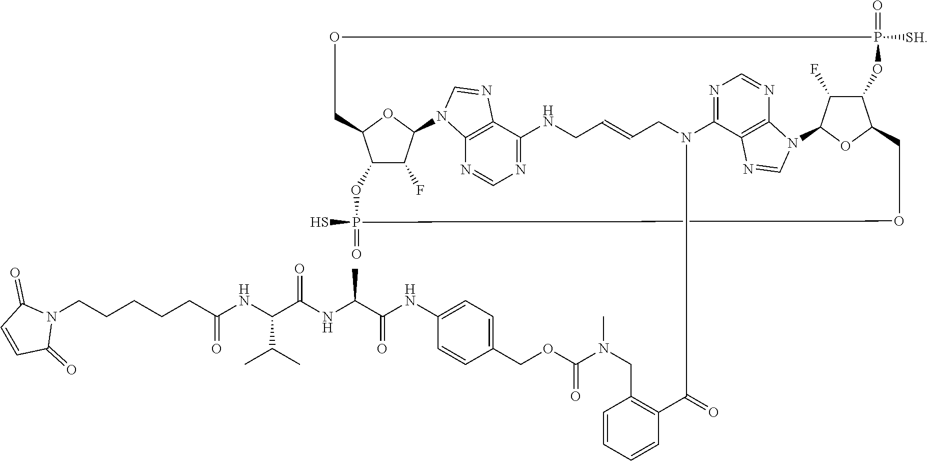 [SMILES: CC(C)[C@H](NC(=O)CCCCCN1C(=O)C=CC1=O)C(=O)N[C@@H](C)C(=O)Nc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

80. The antibody-drug conjugate of embodiment 66, wherein the linker comprises Val-Cit-pABC-Unit 2 moiety.
81. The antibody-drug conjugate of embodiment 66, wherein the linker comprises MC-Val-Cit-pABC-Unit 2 moiety.
82. The antibody-drug conjugate of embodiment 66, wherein the L-D comprises MC-Val-Cit-pABC-Unit 2-Compound 1.
83. The antibody-drug conjugate of embodiment 67, wherein the linker comprises Val-Ala-pABC-Unit 3 moiety.
84. The antibody-drug conjugate of embodiment 67, wherein the linker comprises MC-Val-Ala-pABC-Unit 3 moiety.
85. The antibody-drug conjugate of embodiment 17, wherein the L-D comprises LP20:

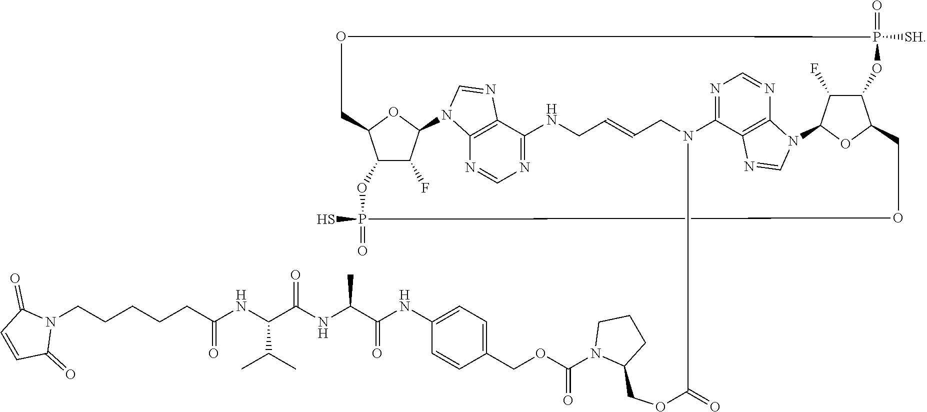 [SMILES: CC(C)[C@H](NC(=O)CCCCCN1C(=O)C=CC1=O)C(=O)N[C@@H](C)C(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

86. The antibody-drug conjugate of embodiment 66, wherein the linker comprises Val-Cit-pABC-Unit 3 moiety.
87. The antibody-drug conjugate of embodiment 66, wherein the linker comprises MC-Val-Cit-pABC-Unit 3 moiety.
88. The antibody-drug conjugate of embodiment 66, wherein the L-D comprises MC-Val-Cit-pABC-Unit 3-Compound 1.
89. The antibody-drug conjugate of embodiment 67, wherein the linker comprises Mal-Formula (II)-Val-Ala-pABC-Unit 2 moiety.
90. The antibody-drug conjugate of embodiment 17, wherein the L-D comprises LP54:

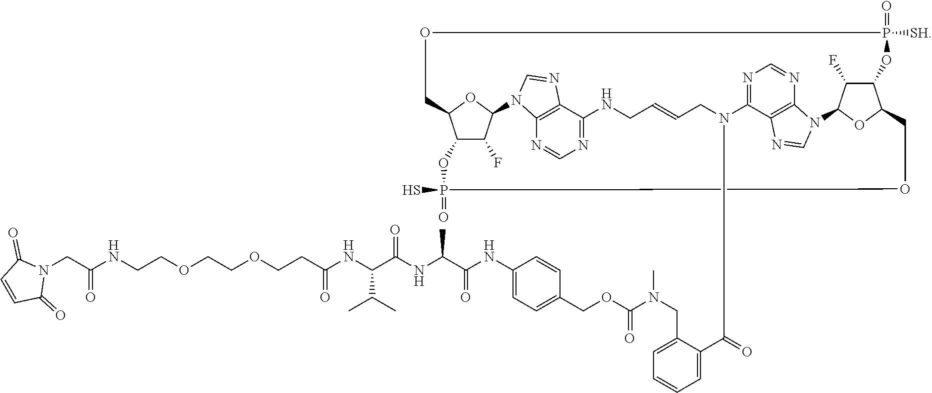 [SMILES: CC(C)[C@H](NC(=O)CCOCCOCCNC(=O)CN1C(=O)C=CC1=O)C(=O)N[C@@H](C)C(=O)Nc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

91. The antibody-drug conjugate of embodiment 66, wherein the linker comprises Mal-Formula (II)-Val-Cit-pABC-Unit 2 moiety.
92. The antibody-drug conjugate of embodiment 67, wherein the linker comprises Mal-(PEG)₂-Val-Ala-pABC-Unit 2 moiety.
93. The antibody-drug conjugate of embodiment 17, wherein the L-D comprises LP55:

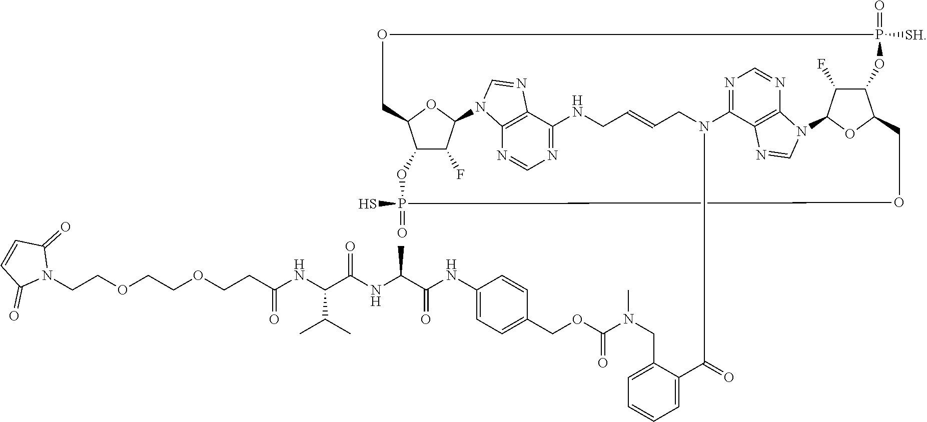 [SMILES: CC(C)[C@H](NC(=O)CCOCCOCCN1C(=O)C=CC1=O)C(=O)N[C@@H](C)C(=O)Nc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

94. The antibody-drug conjugate of embodiment 66, wherein the linker comprises Mal-(PEG)₂-Val-Cit-pABC-Unit 2 moiety.
95. The antibody-drug conjugate of any one of embodiments 17 to 94, wherein the antibody or antigen-binding fragment comprises three HCDRs comprising amino acid sequences of SEQ ID NO: 1 (HCDR1), SEQ ID NO: 2 (HCDR2), and SEQ ID NO: 3 (HCDR3); and three LCDRs comprising amino acid sequences of SEQ ID NO: 4 (LCDR1), SEQ ID NO: 5 (LCDR2), and SEQ ID NO: 6 (LCDR3), as defined by the Kabat numbering system.
96. The antibody-drug conjugate of any one of embodiments 17 to 94, wherein the antibody or antigen-binding fragment comprises three HCDRs comprising amino acid sequences of SEQ ID NO: 7 (HCDR1), SEQ ID NO: 8 (HCDR2), and SEQ ID NO: 9 (HCDR3); and three LCDRs comprising amino acid sequences of SEQ ID NO: 10 (LCDR1), SEQ ID NO: 11 (LCDR2), and SEQ ID NO: 12 (LCDR3), as defined by the IGMT numbering system.
97. The antibody-drug conjugate of embodiment 95 or embodiment 96, wherein the antibody or antigen-binding fragment comprises a heavy chain variable region comprising an amino acid sequence of SEQ ID NO: 49, and a light chain variable region comprising an amino acid sequence of SEQ ID NO: 50.
98. The antibody-drug conjugate of embodiment 95 or embodiment 96, wherein the antibody or antigen-binding fragment comprises a heavy chain variable region comprising an amino acid sequence of SEQ ID NO: 49, and a light chain variable region comprising an amino acid sequence of SEQ ID NO: 51.
99. The antibody-drug conjugate of embodiment 97, wherein the antibody or antigen-binding fragment comprises a heavy chain comprising an amino acid sequence of SEQ ID NO: 63 and a light chain comprising an amino acid sequence of SEQ ID NO: 65.
100. The antibody-drug conjugate of embodiment 97, wherein the antibody or antigen-binding fragment comprises a heavy chain comprising an amino acid sequence of SEQ ID NO: 64 and a light chain comprising an amino acid sequence of SEQ ID NO: 65.
101. The antibody-drug conjugate of embodiment 98, wherein the antibody or antigen-binding fragment comprises a heavy chain comprising an amino acid sequence of SEQ ID NO: 63 and a light chain comprising an amino acid sequence of SEQ ID NO: 66.
102. The antibody-drug conjugate of embodiment 98, wherein the antibody or antigen-binding fragment comprises a heavy chain comprising an amino acid sequence of SEQ ID NO: 64 and a light chain comprising an amino acid sequence of SEQ ID NO: 66.
103. The antibody-drug conjugate of any one of embodiments 17 to 94, wherein the antibody or antigen-binding fragment comprises three HCDRs comprising amino acid sequences of SEQ ID NO: 13 (HCDR1), SEQ ID NO: 14 (HCDR2), and SEQ ID NO: 15 (HCDR3); and three LCDRs comprising amino acid sequences of SEQ ID NO: 16 (LCDR1), SEQ ID NO: 17 (LCDR2), and SEQ ID NO: 18 (LCDR3), as defined by the Kabat numbering system.
104. The antibody-drug conjugate of any one of embodiments 17 to 94, wherein the antibody or antigen-binding fragment comprises three HCDRs comprising amino acid sequences of SEQ ID NO: 19 (HCDR1), SEQ ID NO: 20 (HCDR2), and SEQ ID NO: 21 (HCDR3); and three LCDRs comprising amino acid sequences of SEQ ID NO: 22 (LCDR1), SEQ ID NO: 23 (LCDR2), and SEQ ID NO: 24 (LCDR3), as defined by the IGMT numbering system.
105. The antibody-drug conjugate of embodiment 103 or embodiment 104, wherein the antibody or antigen-binding fragment comprises a heavy chain variable region comprising an amino acid sequence of SEQ ID NO: 52, and a light chain variable region comprising an amino acid sequence of SEQ ID NO: 53.
106. The antibody-drug conjugate of embodiment 103 or embodiment 104, wherein the antibody or antigen-binding fragment comprises a heavy chain variable region comprising an amino acid sequence of SEQ ID NO: 52, and a light chain variable region comprising an amino acid sequence of SEQ ID NO: 54.
107. The antibody-drug conjugate of embodiment 105, wherein the antibody or antigen-binding fragment comprises a heavy chain comprising an amino acid sequence of SEQ ID NO: 69 and a light chain comprising an amino acid sequence of SEQ ID NO: 71.
108. The antibody-drug conjugate of embodiment 105, wherein the antibody or antigen-binding fragment comprises a heavy chain comprising an amino acid sequence of SEQ ID NO: 70 and a light chain comprising an amino acid sequence of SEQ ID NO: 71.
109. The antibody-drug conjugate of embodiment 106, wherein the antibody or antigen-binding fragment comprises a heavy chain comprising an amino acid sequence of SEQ ID NO: 69 and a light chain comprising an amino acid sequence of SEQ ID NO: 72.
110. The antibody-drug conjugate of embodiment 106, wherein the antibody or antigen-binding fragment comprises a heavy chain comprising an amino acid sequence of SEQ ID NO: 70 and a light chain comprising an amino acid sequence of SEQ ID NO: 72.
111. An antibody-drug conjugate of Formula (I): Ab-(L-D), (I)

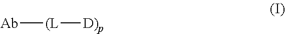

wherein Ab is an internalizing anti-CD73 antibody or internalizing antigen-binding fragment thereof comprising three HCDRs comprising amino acid sequences of SEQ ID NO: 1 (HCDR1), SEQ ID NO: 2 (HCDR2), and SEQ ID NO: 3 (HCDR3); and three LCDRs comprising amino acid sequences of SEQ ID NO: 4 (LCDR1), SEQ ID NO: 5 (LCDR2), and SEQ ID NO: 6 (LCDR3), as defined by the Kabat numbering system; or three HCDRs comprising amino acid sequences of SEQ ID NO: 7 (HCDR1), SEQ ID NO: 8 (HCDR2), and SEQ ID NO: 9 (HCDR3); and three LCDRs comprising amino acid sequences of SEQ ID NO: 10 (LCDR1), SEQ ID NO: 11 (LCDR2), and SEQ ID NO: 12 (LCDR3), as defined by the IGMT numbering system;
p is an integer from 1 to 8; and
L-D comprises LP2:

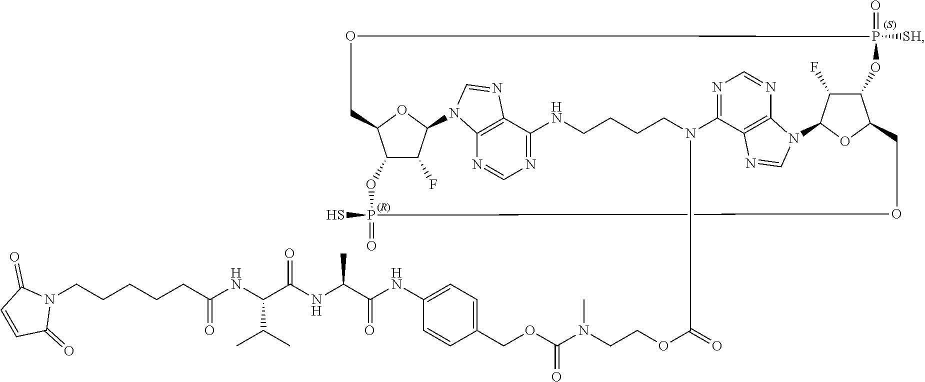

or a salt thereof.
112. An antibody-drug conjugate of Formula (I):

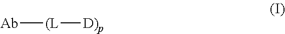

wherein Ab is an internalizing anti-CD73 antibody or internalizing antigen-binding fragment thereof comprising three HCDRs comprising amino acid sequences of SEQ ID NO: 1 (HCDR1), SEQ ID NO: 2 (HCDR2), and SEQ ID NO: 3 (HCDR3); and three LCDRs comprising amino acid sequences of SEQ ID NO: 4 (LCDR1), SEQ ID NO: 5 (LCDR2), and SEQ ID NO: 6 (LCDR3), as defined by the Kabat numbering system; or three HCDRs comprising amino acid sequences of SEQ ID NO: 7 (HCDR1), SEQ ID NO: 8 (HCDR2), and SEQ ID NO: 9 (HCDR3); and three LCDRs comprising amino acid sequences of SEQ ID NO: 10 (LCDR1), SEQ ID NO: 11 (LCDR2), and SEQ ID NO: 12 (LCDR3), as defined by the IGMT numbering system;
p is an integer from 1 to 8; and
L-D comprises LP16:

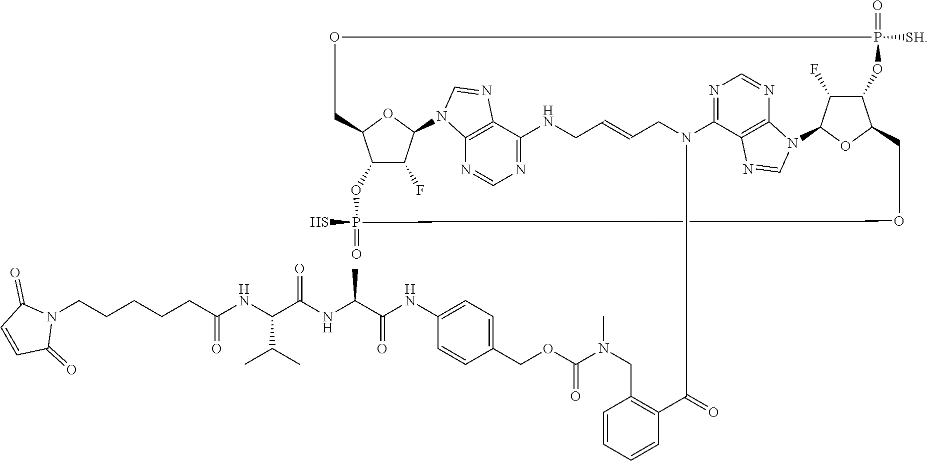 [SMILES: CC(C)[C@H](NC(=O)CCCCCN1C(=O)C=CC1=O)C(=O)N[C@@H](C)C(=O)Nc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

or a salt thereof.
113. An antibody-drug conjugate of Formula (I):

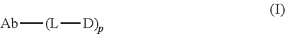

wherein Ab is an internalizing anti-CD73 antibody or internalizing antigen-binding fragment thereof comprising three HCDRs comprising amino acid sequences of SEQ ID NO: 1 (HCDR1), SEQ ID NO: 2 (HCDR2), and SEQ ID NO: 3 (HCDR3); and three LCDRs comprising amino acid sequences of SEQ ID NO: 4 (LCDR1), SEQ ID NO: 5 (LCDR2), and SEQ ID NO: 6 (LCDR3), as defined by the Kabat numbering system; or three HCDRs comprising amino acid sequences of SEQ ID NO: 7 (HCDR1), SEQ ID NO: 8 (HCDR2), and SEQ ID NO: 9 (HCDR3); and three LCDRs comprising amino acid sequences of SEQ ID NO: 10 (LCDR1), SEQ ID NO: 11 (LCDR2), and SEQ ID NO: 12 (LCDR3), as defined by the IGMT numbering system;
p is an integer from 1 to 8; and
L-D comprises LP20:

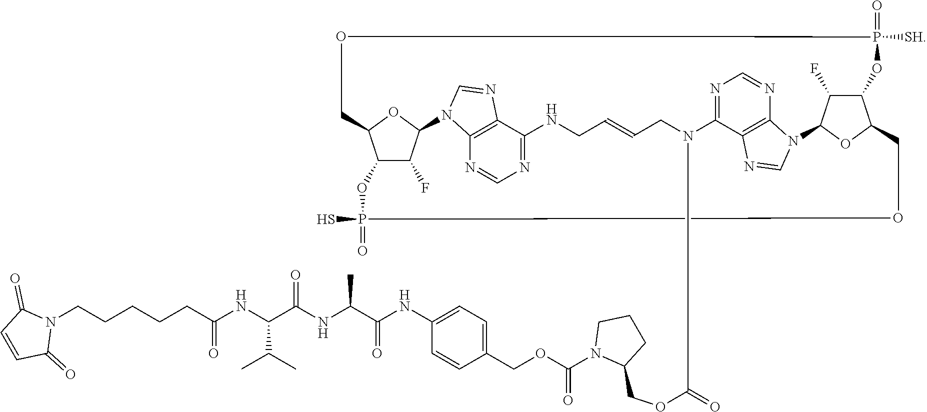 [SMILES: CC(C)[C@H](NC(=O)CCCCCN1C(=O)C=CC1=O)C(=O)N[C@@H](C)C(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

or a salt thereof.
114. An antibody-drug conjugate of Formula (I):

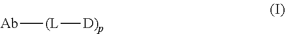

wherein Ab is an internalizing anti-CD73 antibody or internalizing antigen-binding fragment thereof comprising three HCDRs comprising amino acid sequences of SEQ ID NO: 1 (HCDR1), SEQ ID NO: 2 (HCDR2), and SEQ ID NO: 3 (HCDR3); and three LCDRs comprising amino acid sequences of SEQ ID NO: 4 (LCDR1), SEQ ID NO: 5 (LCDR2), and SEQ ID NO: 6 (LCDR3), as defined by the Kabat numbering system; or three HCDRs comprising amino acid sequences of SEQ ID NO: 7 (HCDR1), SEQ ID NO: 8 (HCDR2), and SEQ ID NO: 9 (HCDR3); and three LCDRs comprising amino acid sequences of SEQ ID NO: 10 (LCDR1), SEQ ID NO: 11 (LCDR2), and SEQ ID NO: 12 (LCDR3), as defined by the IGMT numbering system;
p is an integer from 1 to 8; and
L-D comprises LP54:

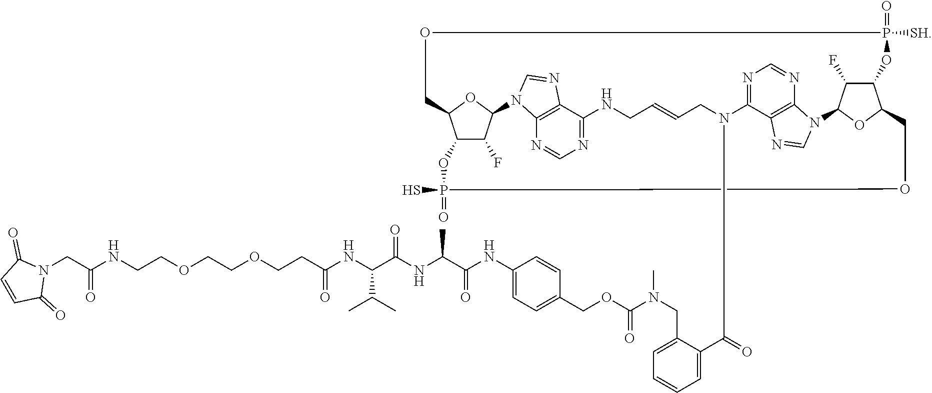 [SMILES: CC(C)[C@H](NC(=O)CCOCCOCCNC(=O)CN1C(=O)C=CC1=O)C(=O)N[C@@H](C)C(=O)Nc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

or a salt thereof.
115. An antibody-drug conjugate of Formula (I):

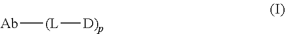

wherein Ab is an internalizing anti-CD73 antibody or internalizing antigen-binding fragment thereof comprising three HCDRs comprising amino acid sequences of SEQ ID NO: 1 (HCDR1), SEQ ID NO: 2 (HCDR2), and SEQ ID NO: 3 (HCDR3); and three LCDRs comprising amino acid sequences of SEQ ID NO: 4 (LCDR1), SEQ ID NO: 5 (LCDR2), and SEQ ID NO: 6 (LCDR3), as defined by the Kabat numbering system; or three HCDRs comprising amino acid sequences of SEQ ID NO: 7 (HCDR1), SEQ ID NO: 8 (HCDR2), and SEQ ID NO: 9 (HCDR3); and three LCDRs comprising amino acid sequences of SEQ ID NO: 10 (LCDR1), SEQ ID NO: 11 (LCDR2), and SEQ ID NO: 12 (LCDR3), as defined by the IGMT numbering system;
p is an integer from 1 to 8; and
L-D comprises LP55:

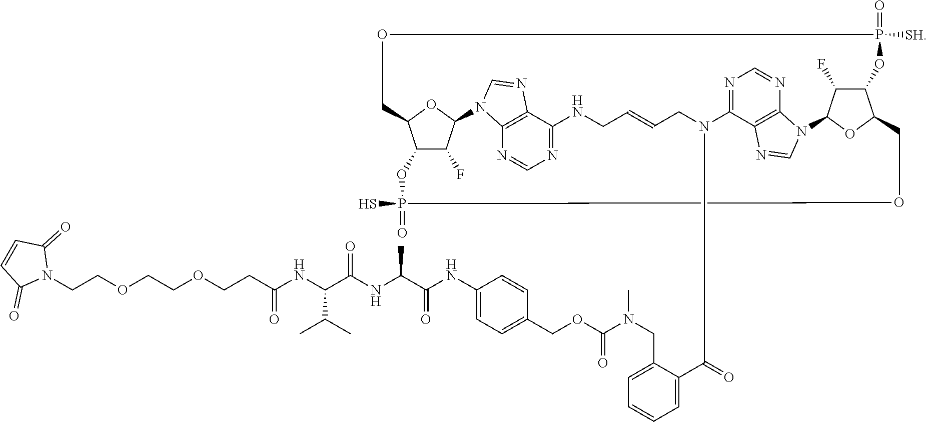 [SMILES: CC(C)[C@H](NC(=O)CCOCCOCCN1C(=O)C=CC1=O)C(=O)N[C@@H](C)C(=O)Nc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

or a salt thereof.
116. The antibody-drug conjugate of any one of embodiments 111 to 115, wherein the antibody or antigen-binding fragment comprises a heavy chain variable region comprising an amino acid sequence of SEQ ID NO: 49, and a light chain variable region comprising an amino acid sequence of SEQ ID NO: 50.
117. The antibody-drug conjugate of any one of embodiments 111 to 115, wherein the antibody or antigen-binding fragment comprises a heavy chain variable region comprising an amino acid sequence of SEQ ID NO: 49, and a light chain variable region comprising an amino acid sequence of SEQ ID NO: 51.
118. An antibody-drug conjugate of Formula (I):

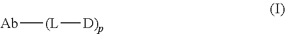

wherein Ab is an internalizing anti-CD73 antibody or internalizing antigen-binding fragment thereof comprising three HCDRs comprising amino acid sequences of SEQ ID NO: 13 (HCDR1), SEQ ID NO: 14 (HCDR2), and SEQ ID NO: 15 (HCDR3); and three LCDRs comprising amino acid sequences of SEQ ID NO: 16 (LCDR1), SEQ ID NO: 17 (LCDR2), and SEQ ID NO: 18 (LCDR3), as defined by the Kabat numbering system; or three HCDRs comprising amino acid sequences of SEQ ID NO: 19 (HCDR1), SEQ ID NO: 20 (HCDR2), and SEQ ID NO: 21 (HCDR3); and three LCDRs comprising amino acid sequences of SEQ ID NO: 22 (LCDR1), SEQ ID NO: 23 (LCDR2), and SEQ ID NO: 24 (LCDR3), as defined by the IGMT numbering system;
p is an integer from 1 to 8; and
L-D comprises LP2:

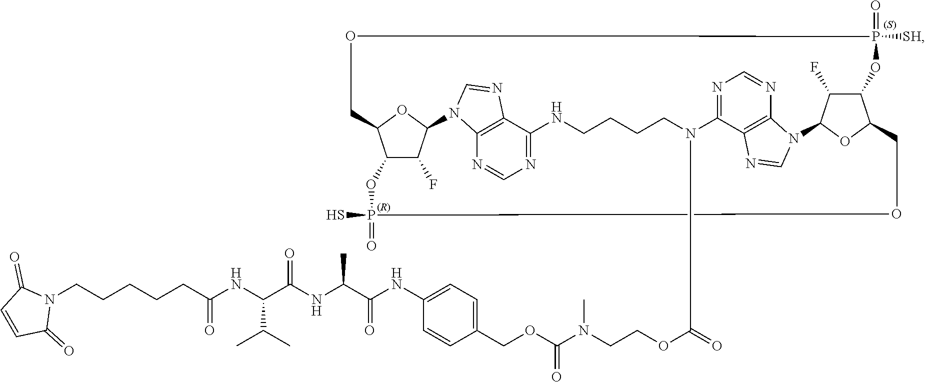

or a salt thereof.
119. An antibody-drug conjugate of Formula (I):

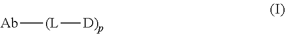

wherein Ab is an internalizing anti-CD73 antibody or internalizing antigen-binding fragment thereof comprising three HCDRs comprising amino acid sequences of SEQ ID NO: 13 (HCDR1), SEQ ID NO: 14 (HCDR2), and SEQ ID NO: 15 (HCDR3); and three LCDRs comprising amino acid sequences of SEQ ID NO: 16 (LCDR1), SEQ ID NO: 17 (LCDR2), and SEQ ID NO: 18 (LCDR3), as defined by the Kabat numbering system; or three HCDRs comprising amino acid sequences of SEQ ID NO: 19 (HCDR1), SEQ ID NO: 20 (HCDR2), and SEQ ID NO: 21 (HCDR3); and three LCDRs comprising amino acid sequences of SEQ ID NO: 22 (LCDR1), SEQ ID NO: 23 (LCDR2), and SEQ ID NO: 24 (LCDR3), as defined by the IGMT numbering system;
p is an integer from 1 to 8; and
L-D comprises LP16:

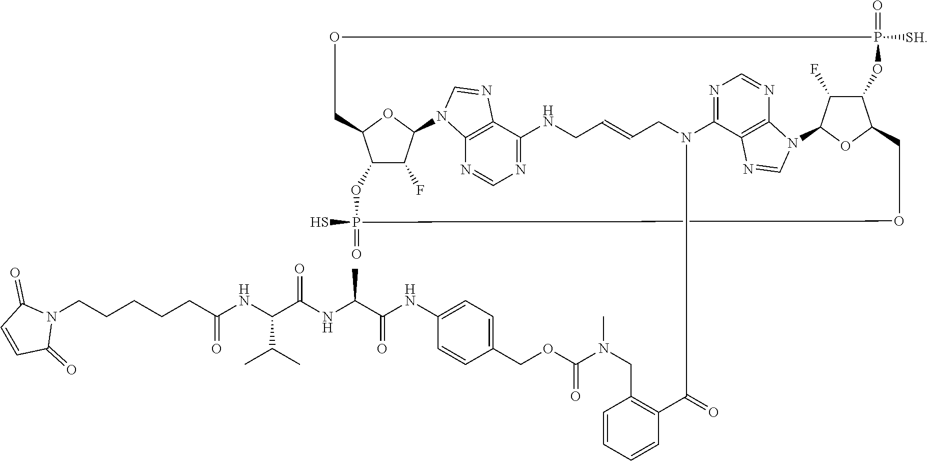 [SMILES: CC(C)[C@H](NC(=O)CCCCCN1C(=O)C=CC1=O)C(=O)N[C@@H](C)C(=O)Nc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

or a salt thereof.
120. An antibody-drug conjugate of Formula (I):

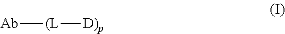

wherein Ab is an internalizing anti-CD73 antibody or internalizing antigen-binding fragment thereof comprising three HCDRs comprising amino acid sequences of SEQ ID NO: 13 (HCDR1), SEQ ID NO: 14 (HCDR2), and SEQ ID NO: 15 (HCDR3); and three LCDRs comprising amino acid sequences of SEQ ID NO: 16 (LCDR1), SEQ ID NO: 17 (LCDR2), and SEQ ID NO: 18 (LCDR3), as defined by the Kabat numbering system; or three HCDRs comprising amino acid sequences of SEQ ID NO: 19 (HCDR1), SEQ ID NO: 20 (HCDR2), and SEQ ID NO: 21 (HCDR3); and three LCDRs comprising amino acid sequences of SEQ ID NO: 22 (LCDR1), SEQ ID NO: 23 (LCDR2), and SEQ ID NO: 24 (LCDR3), as defined by the IGMT numbering system;
p is an integer from 1 to 8; and
L-D comprises LP20:

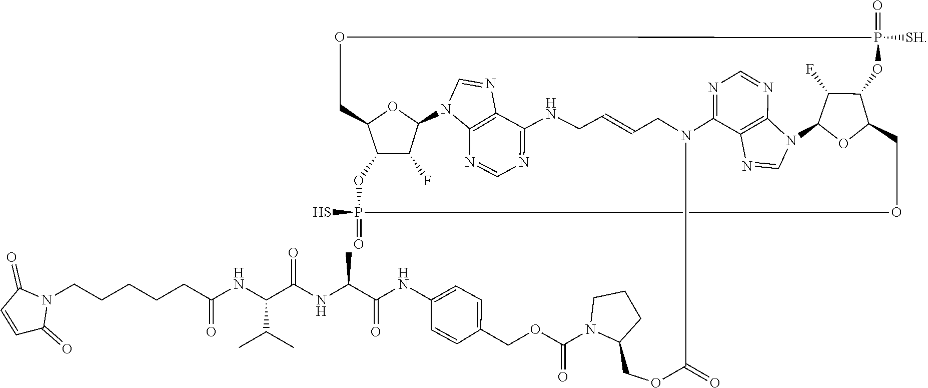 [SMILES: CC(C)[C@H](NC(=O)CCCCCN1C(=O)C=CC1=O)C(=O)N[C@@H](C)C(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

a salt thereof.
121. An antibody-drug conjugate of Formula (I):

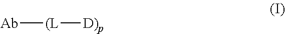

wherein Ab is an internalizing anti-CD73 antibody or internalizing antigen-binding fragment thereof comprising three HCDRs comprising amino acid sequences of SEQ ID NO: 13 (HCDR1), SEQ ID NO: 14 (HCDR2), and SEQ ID NO: 15 (HCDR3); and three LCDRs comprising amino acid sequences of SEQ ID NO: 16 (LCDR1), SEQ ID NO: 17 (LCDR2), and SEQ ID NO: 18 (LCDR3), as defined by the Kabat numbering system; or three HCDRs comprising amino acid sequences of SEQ ID NO: 19 (HCDR1), SEQ ID NO: 20 (HCDR2), and SEQ ID NO: 21 (HCDR3); and three LCDRs comprising amino acid sequences of SEQ ID NO: 22 (LCDR1), SEQ ID NO: 23 (LCDR2), and SEQ ID NO: 24 (LCDR3), as defined by the IGMT numbering system;
p is an integer from 1 to 8; and
L-D comprises LP54:

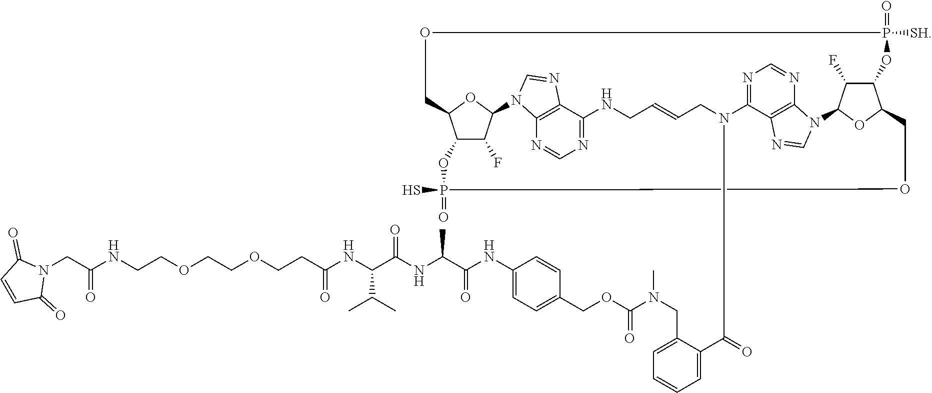 [SMILES: CC(C)[C@H](NC(=O)CCOCCOCCNC(=O)CN1C(=O)C=CC1=O)C(=O)N[C@@H](C)C(=O)Nc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

or a salt thereof.
122. An antibody-drug conjugate of Formula (I):

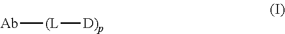

wherein Ab is an internalizing anti-CD73 antibody or internalizing antigen-binding fragment thereof comprising three HCDRs comprising amino acid sequences of SEQ ID NO: 13 (HCDR1), SEQ ID NO: 14 (HCDR2), and SEQ ID NO: 15 (HCDR3); and three LCDRs comprising amino acid sequences of SEQ ID NO: 16 (LCDR1), SEQ ID NO: 17 (LCDR2), and SEQ ID NO: 18 (LCDR3), as defined by the Kabat numbering system; or three HCDRs comprising amino acid sequences of SEQ ID NO: 19 (HCDR1), SEQ ID NO: 20 (HCDR2), and SEQ ID NO: 21 (HCDR3); and three LCDRs comprising amino acid sequences of SEQ ID NO: 22 (LCDR1), SEQ ID NO: 23 (LCDR2), and SEQ ID NO: 24 (LCDR3), as defined by the IGMT numbering system;
p is an integer from 1 to 8; and
L-D comprises LP55:

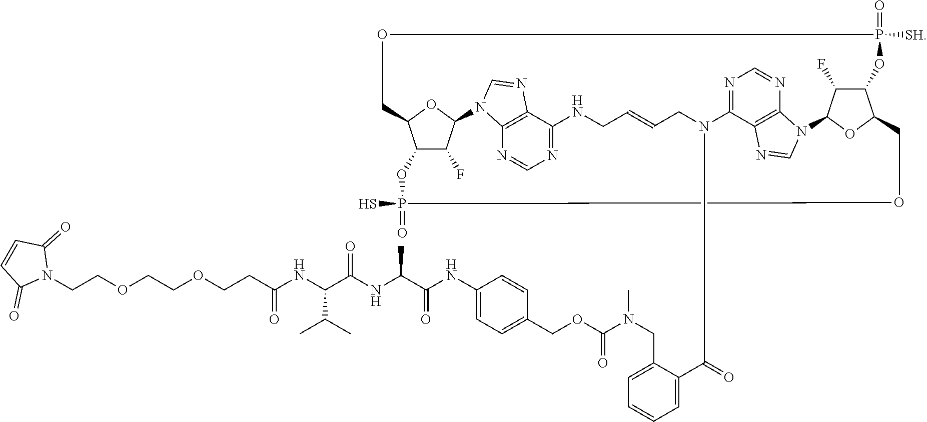 [SMILES: CC(C)[C@H](NC(=O)CCOCCOCCN1C(=O)C=CC1=O)C(=O)N[C@@H](C)C(=O)Nc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

or a salt thereof.
123. The antibody-drug conjugate of any one of embodiments 118 to 122, wherein the antibody or antigen-binding fragment comprises a heavy chain variable region comprising an amino acid sequence of SEQ ID NO: 52, and a light chain variable region comprising an amino acid sequence of SEQ ID NO: 53.
124. The antibody-drug conjugate of any one of embodiments 118 to 122, wherein the antibody or antigen-binding fragment comprises a heavy chain variable region comprising an amino acid sequence of SEQ ID NO: 52, and a light chain variable region comprising an amino acid sequence of SEQ ID NO: 54.
125. The antibody-drug conjugate of any one of embodiments 111 to 124, wherein p is 2 or 4.
126. The antibody-drug conjugate of any one of embodiments 17 to 125, wherein p is determined by hydrophobic interaction chromatography-high performance liquid chromatography (HIC-HPLC).
127. The antibody-drug conjugate of any one of embodiments 17 to 125, wherein p is determined by reverse-phase liquid chromatography-mass spectrometry (LC-MS).
128. A composition comprising multiple copies of the antibody-drug conjugate of any one of embodiments 17 to 127, wherein the average p of the antibody-drug conjugates in the composition is about 1 to about 4.
129. The composition of embodiment 128, wherein the average p is about 2 or about 4.
130. A pharmaceutical composition comprising the antibody or antigen-binding fragment of any one of embodiments 1 to 16, the antibody-drug conjugate of any one of embodiments 17 to 127, or the composition of embodiment 128 or embodiment 129, and a pharmaceutically acceptable carrier.
131. A method of treating a patient having or at risk of having a cancer that expresses CD73, comprising administering to the patient a therapeutically effective amount of the antibody or antigen-binding fragment of any one of embodiments 1 to 16, the antibody-drug conjugate of any one of embodiments 17 to 127, the composition of embodiment 128 or embodiment 129, or the pharmaceutical composition of embodiment 130.
132. The method of embodiment 131, wherein the CD73-expressing cancer is melanoma, diffuse large B-cell lymphoma, T-cell lymphoma, breast cancer, ovarian cancer, head and neck cancer, head and neck squamous carcinoma, non-small-cell lung cancer (NSCLC), glioblastoma, thyroid carcinoma, pancreatic cancer, pancreatic ductal adenocarcinoma (PDAC), esophageal cancer, cervical cancer, gastric cancer, gallbladder cancer, or colorectal cancer.
133. The method of embodiment 132, wherein the CD73-expressing cancer is a breast cancer, ovarian cancer, PDAC, or NSCLC.
134. A method of stimulating an anti-tumor immune response in the tumor microenvironment of a patient having a cancer that expresses CD73, comprising administering to the patient a therapeutically effective amount of the antibody or antigen-binding fragment of any one of embodiments 1 to 16, the antibody-drug conjugate of any one of embodiments 17 to 127, the composition of embodiment 128 or embodiment 129, or the pharmaceutical composition of embodiment 130.
135. A method of reducing or inhibiting growth of a CD73-expressing tumor, comprising administering a therapeutically effective amount of an antibody or antigen-binding fragment of any one of embodiments 1 to 16, the antibody-drug conjugate of any one of embodiments 17 to 127, the composition of embodiment 128 or embodiment 129, or the pharmaceutical composition of embodiment 130.
136. The method of embodiment 135, wherein the tumor is a CD73-expressing melanoma, diffuse large B-cell lymphoma, T-cell lymphoma, breast cancer, ovarian cancer, head and neck cancer, head and neck squamous carcinoma, non-small-cell lung cancer (NSCLC), glioblastoma, thyroid carcinoma, pancreatic cancer, pancreatic ductal adenocarcinoma (PDAC), esophageal cancer, cervical cancer, gastric cancer, gallbladder cancer, or colorectal cancer.
137. The method of embodiment 136, wherein the CD73-expressing cancer is a breast cancer, ovarian cancer, PDAC, or NSCLC.
138. Use of an antibody or antigen-binding fragment of any one of embodiments 1 to 16, the antibody-drug conjugate of any one of embodiments 17 to 127, the composition of embodiment 128 or embodiment 129, or the pharmaceutical composition of embodiment 130 in the manufacture of a medicament for the treatment of a CD73-expressing cancer.
139. Use of an antibody or antigen-binding fragment of any one of embodiments 1 to 16, the antibody-drug conjugate of any one of embodiments 17 to 127, the composition of embodiment 128 or embodiment 129, or the pharmaceutical composition of embodiment 130 in the treatment of a CD73-expressing cancer.
140. The use of embodiment 138 or embodiment 139, wherein the CD73-expressing cancer is CD73-expressing melanoma, diffuse large B-cell lymphoma, T-cell lymphoma, breast cancer, ovarian cancer, head and neck cancer, head and neck squamous carcinoma, non-small-cell lung cancer (NSCLC), glioblastoma, thyroid carcinoma, pancreatic cancer, pancreatic ductal adenocarcinoma (PDAC), esophageal cancer, cervical cancer, gastric cancer, gallbladder cancer, or colorectal cancer.
141. The use of embodiment 140, wherein the CD73-expressing cancer is a breast cancer, ovarian cancer, PDAC, or NSCLC.
142. Nucleic acid(s) encoding the antibody or antigen-binding fragment of any one of embodiments 1 to 14.
143. A host cell comprising the nucleic acid(s) of embodiment 142.
144. A method of producing an antibody of any one of embodiments 1 to 14, comprising culturing the host cell of embodiment 143 under conditions sufficient to produce the antibody or antigen-binding fragment.
145. A method of producing the antibody-drug conjugate of any one of embodiments 17 to 127 or the composition of embodiment 128 or embodiment 129, comprising reacting an antibody or antigen-binding fragment of any one of embodiments 1 to 14 with a cleavable linker joined to D under conditions that allow conjugation, wherein D comprises a compound selected from:

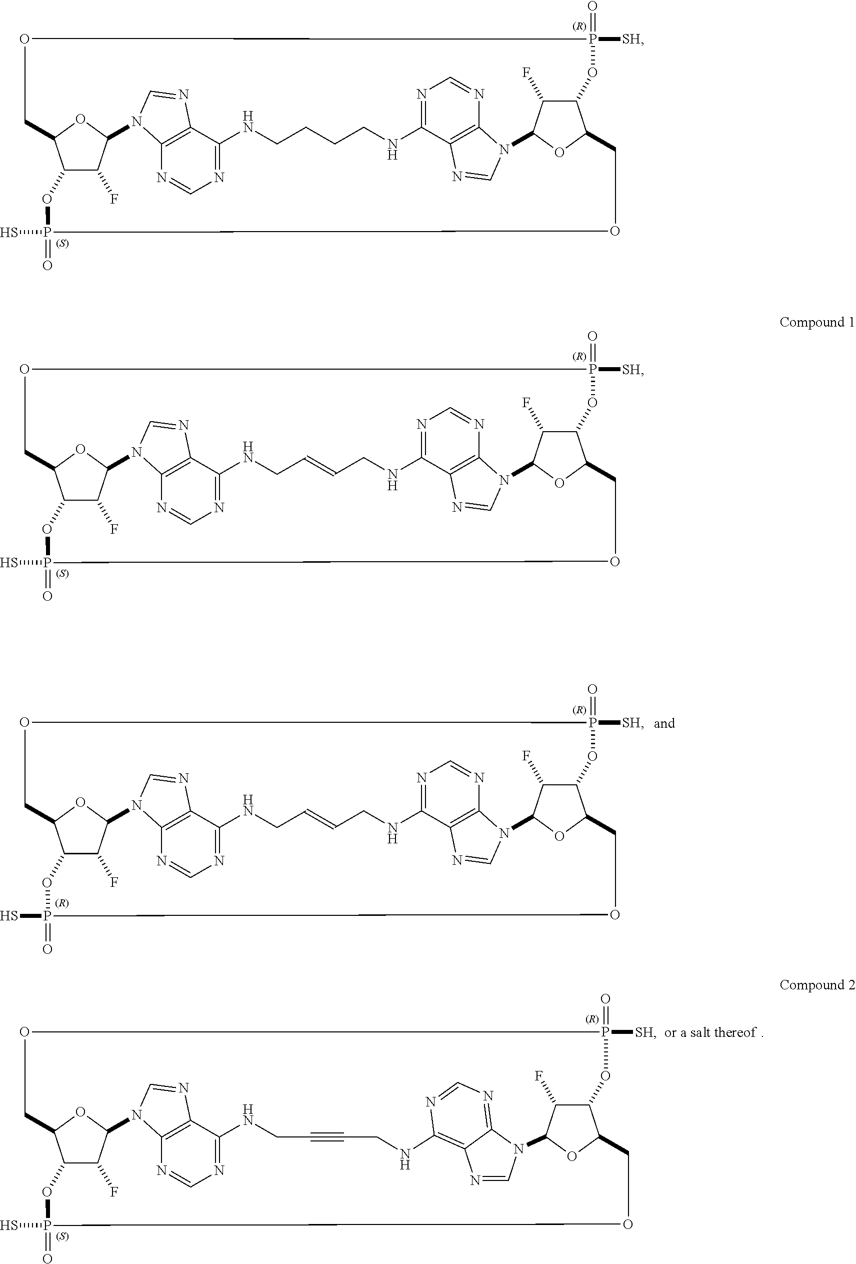

146. An antibody-drug conjugate produced according to the method of embodiment 145.

It will be readily apparent to those skilled in the art that other suitable modifications and adaptations of the methods of the invention described herein are obvious and may be made using suitable equivalents without departing from the scope of the invention or the embodiments disclosed herein. Having now described the invention in detail, the same will be more clearly understood by reference to the following examples, which are included for purposes of illustration only and are not intended to be limiting.

### EXAMPLES

### Example 1: Methods

The following examples employed methods that are detailed below.

### Mouse Immunization, Screening, and Antibody Selection

Due to the high identity between human and mouse CD73, CD73-knockout mice were used for DNA immunization to increase CD73 immunogenicity. Full-length human CD73 complementary DNA (cDNA) was cloned into an Aldevron proprietary immunization plasmid (pB8) to produce CD73 expression plasmid pB8-CD73, which is tagged. Five CD73 knockout (KO) mice were immunized with pB8-CD73 plasmid four times. The sera from these mice were taken at day 24, and the titer was measured by fluorescence-activated cell sorting (FACS) using mammalian cells transiently transfected with pB8-CD73 or an irrelevant control construct (pB8-X). After reactivity to CD73 was detected in the sera, the mice were sacrificed, and the lymphocytes from spleens and lymph nodes were isolated. The lymphocytes from spleens and lymph nodes subjected to single B cell culture in a 384-well plate produce enough IgG for screening and to isolate RNA for Ig variable region rescue.

For primary screening, all supernatants were screened by mouse IgG competitive Homogeneous Time Resolved Fluorescence (HTRF) assay. Briefly, 20 μL/well of supernatant was transferred to the assay plates and then 20 μL/well HTRF complex (25 ng/mL of Europium cryptate-labeled anti-mouse IgG+M with Cy5 conjugated Mouse IgG in complex buffer) was added. The plates were covered with foil and incubated at room temperature for 4 hours, and then read at 620 nm and 665 nm on the Beckman Coulter paradigm plate readers. HTRF ratio (Reading at 665 nm/Reading at 620 nm*10000) of each sample was calculated and normalized to positive and negative controls. 100 ng/mL mouse IgG was used as the positive control and medium as the negative control.

After IgG-producing wells were identified, supernatants from these wells were consolidated into new 384-well plates and subjected to secondary screening. This was accomplished by high-throughput FACS (Hypercyte) analysis using Jurkat (control cells), BNT5.1 (Jurkat expressing human CD73), and JC-1 cells (Jurkat expressing murine CD73) in a multiplexed single-well format. Briefly, Jurkat and JC-1 cells were pre-stained with 1 μM and 40 nM Cell Tracker Green CMFDA, respectively. 5000 each of Jurkat, BNT5.1, and JC-1 cells were mixed with 500 ng/mL of AlexaFluor647-conjugated goat anti-mouse IgG Fcγ in a total of 10 μL in each well of FACS plates, and shaken on a Hypercyte shaker at 2800 rpm for 5 seconds. Then, 10 μl of the consolidated supernatant was added to the plates and incubated at room temperature for 2 hours. 5 μl of PBS-diluted formaldehyde was added to all wells to fix the cells (final formaldehyde was 1.5%). The plates were shaken on a Hypercyte shaker at 2800 rpm for 5 seconds and read on Hypercyte.

In a secondary screening, the clones were selected based on the following criteria: 1) no reactivity to Jurkat cells (no CD73 expression) and 2) reactivity to BNT5.1 cells (human CD73). It is noteworthy that some of the selected clones also displayed reactivity to JC-1 cells, suggesting that these clones are dually reactive to human and murine CD73.

Cells from selected clones were lysed and total RNAs isolated with RNAqueous Kit (Ambion). mRNAs were bound to Dynabeads® Oligo (dT)25 (SEQ ID NO: 254) (Ambion) and reverse transcribed with ThermoScript Reverse Transcriptase (Invitrogen) at 50° C. for 5 minutes followed by 60° C. for 1 hour. The resulting cDNA were treated with terminal deoxynucleotidyl transferase by incubating 10 minutes at 37° C. followed by inactivation of enzyme by heating to 70° C. for 10 minutes. This added homopolynucleotide to the 3′ end of cDNA so that adaptor PCR could be used to amplify the 5′ end of mRNA without needing to know its sequence. Then, two rounds of PCR were performed.

After PCR, PCR products were subjected to agarose electrophoresis. Both mouse heavy and light chain bands were identified and purified with QIAquick 96 PCR Purification Kit (Qiagen) according to the manufacturer's instructions. PCR fragments were sequenced and analyzed with IMGT database tools to confirm the identity of each clone.

### Rabbit Immunization, Screening, and Antibody Selection

Two rabbits were immunized by subcutaneous injection with the CD73-ECD antigens every 2 weeks for a total of five times. The pre- and test-bleeds were collected for antibody titer testing. Specific titers were measured by FACS using MDA-MB-231 and Jurkat cell lines expressing human CD73 (BNT5.1) or mouse CD73 (JC-1) respectively. Rabbits were sacrificed and spleen cells were isolated from each rabbit and cryopreserved.

Cryo-conserved rabbit lymph node cells (2.0×10⁷ cells) were thawed, then activated with 2.5 g/mL of lectin from Phytolacca americana and recovered with DNAse I for one hour at 37° C. with 5% CO₂. The cells were seeded at 5 cells per well on a 384 well plate with feeder cells (CHOs expressing rabbit CD154) and cultured in complete IMDM (i.e., IMDM supplemented with 10% FBS, 2 mM L-glutamine, 1×MEM NEAA, 1 mM sodium pyruvate, 50 U/mL penicillin, 50 g/mL streptomycin, and 55 μM 2-Me) that contained 10.5 ng/mL human IL-2 and 10.5 ng/mL human IL-21 cytokines (PeproTech).

On week two, the wells producing rabbit IgG antibody were identified by IgG HTRF as described above. Wells producing IgG were screened for the presence of anti-CD73 reactive antibodies by high-throughput FACS screening as described above.

Total RNA was isolated from wells producing rabbit IgG anti-CD73 antibodies using RNAqueous™-96 Total RNA Isolation Kit (Ambion). cDNA was synthesized and light and heavy chain variable regions were amplified by PCR using Platinum Taq one-step RT-PCR kit (Invitrogen) using in-house primers. The light and heavy chain variable regions were amplified with nested primers using Platinum Taq Amplification Kit and a thermocycler. Amplified DNA template was visualized by gel electrophoresis, purified by QIAquick 96 PCR Purification Kit (Qiagen) and DNA sequence was determined by GeneWiz (South Plainfield, NJ) using in-house primers. DNA sequences were analyzed using V-gene and J-gene rabbit families (IMGT/V-QUEST).

### In Silico Modeling for Humanization

The amino acid sequences of anti-CD73 mAbs were analyzed using a BLAST search against a human variable domain database at http://www.ncbi.nlm.nih.gov/igblast/to identify the human sequence with highest homology to the mouse or rabbit sequences. The sequences corresponding to the antigen binding domains as identified by Kabat and Chothia CDRH1, Chothia CDRH2, CDRH3, CDRL1, CDRL2, and CDRL3 were grafted into the framework (FWR) regions of the most homologous human framework.

The variable domains of the mouse or rabbit and CDR-grafted sequences were used to generate in silico structural models. The most homologous mouse/rabbit or human variable domain crystal structures were identified using BLAST pdb database and were used as a template for modeling. Models were generated using Biovia Discovery Studio or Schrodinger BioLuminate. The mouse/rabbit and humanized models for each mAb were superimposed and differing amino acids were highlighted. Those residues adjacent to the CDRs were analyzed to determine whether they were predicted to influence the structure of the antigen-binding site.

### Cloning and Mutagenesis of Recombinant Antibodies

PCR fragments encoding the variable heavy (VH) and variable light (VL) chains of antibodies isolated from mice or rabbits were used to generate full-length antibodies. Humanized variable domain DNAs were codon optimized for expression and synthesized. The mice, rabbit, or humanized gene fragments were subcloned into an expression plasmid containing a human gamma or kappa constant region using an InFusion HD cloning kit (Clontech). All clones were sequenced to confirm the presence and fidelity of the inserts.

Point mutations were made using Stratagene's QuikChange XL according to the manufacturer's protocol. All clones were sequenced to confirm the presence of the mutation.

### Methods of Antibody Production and Purification

### Cell Culture: Transfection and Stable Cell Line Generation

One day prior to transfection, expi293 cells were seeded at 2×10⁶ cells/mL in expi293 medium (ThermoFischer) in a shake flask and incubated at 37° C., 8% CO₂, with shaking at 125 rpm. On the day of transfection, cells were seeded at 3×10⁶ cells/mL as above. Cells were transfected by incubating 333.3 ng HC plasmid and 333.3 ng LC plasmid for 5-10 minute in 50 μL Opti-MEM (ThermoFischer) per mL of transfected cells. Likewise, 2.67 μL ExpiFectamine was incubated in 50 μL Opti-MEM. The ExpiFectamine solution was added to the DNA mixture and incubated for 20-30 minutes at room temperature. The DNA:ExpiFectamine mixture was added to the cells while swirling and incubated at 37° C., 8% CO₂, shaking at 125 rpm. The following day, 3 μL of enhancer 1 and 30 μL of enhancer 2 per mL of cells were added to the transfection with continued incubation for another 7-10 days.

Antibody-expressing stable pools were selected by adding 3 mL of transfectants to 12 mL DMEM in a T75 flask with 5 μg/mL blasticidin and 400 μg/mL zeocin (ThermoFischer) one to three days after transfection. After drug-resistant cells grew to confluency, the medium was replaced with FreeStyle 293 expression medium for 24-48 hours. Cells were physically dislodged by tapping the flask and were then seeded at 6×10⁵ cells/mL in 30 mL FreeStyle 293 expression medium in a 125-mL shake flask. Cultures were incubated at 37° C. in 8% CO₂ with shaking at 125 rpm.

### mAB Production

Antibody production from stable pools was performed by seeding cells at 0.6 to 1×10⁶ cells/mL in FreeStyle 293 expression medium. Two days after the culture reached a density of 1×10⁶ cells/mL, cultures were fed at a final concentration of 10 g/L Yeastolate (BD Biosciences), 5 mM valeric acid (Sigma Aldrich), and 1:100 CD Lipid Concentrate (ThermoFischer). Cells were incubated at 37° C., 8% CO₂, shaking at 125 rpm for 7-10 days. When the cell viability was less than 50%, the cultures were centrifuged for 1 h at 8000 rpm in a Beckman JLA8.1000 rotor. The supernatant was then filtered through a 0.2 μm PES filter and stored at 4° C. or −20° C. until purification.

### Protein A Antibody Purification

Appropriate size Mabselect sure column (Cytivia) was equilibrated with 6 column volumes (CV) of 20 mM Sodium Phosphate, pH 7.2. The conditioned medium containing antibody was filtered through a 0.2 μm membrane and then loaded to the column, followed by washing unbound material with 10 CV of Equilibration buffer. The sample was eluted using 5 CV of 0.1 M Glycine pH 2.9. Eluted material was loaded on to a 26/10 HiPrep desalting column (GE Healthcare) equilibrated in 1× phosphate-buffered saline (PBS) and eluted in the same buffer. Peak fractions were pooled and filtered. Samples were buffer exchanged into 1×PBS.

### Decysteinylation Protein A Purification

Appropriate size Mabselect sure column (Cytivia) was equilibrated with 6 CV of 20 mM Sodium Phosphate, 10 mM EDTA (ethylene diamine tetra acetic acid), pH 7.2. The conditioned medium containing antibody was filtered through a 0.2 μm membrane and then loaded to the column, followed by washing unbound material with 10 CV of Equilibration buffer. The column was washed with 20 mM sodium phosphate buffer containing 10 mM EDTA, 10 mM Cysteine, pH 7.2 for 16 hours at a low flow rate. This step was followed by another wash with 20 mM Tris, pH 7.5 for 60 hours at a low flow rate. The sample was eluted using 5 CV of 0.1 M Glycine pH 2.9. Eluted material was loaded on to a 26/10 HiPrep desalting column (GE Healthcare) equilibrated in 1×PBS and eluted in the same buffer. Peak fractions were pooled and filtered. Samples were buffer exchanged into 1×PBS.

### Antibody and Linker Payload Conjugation Methods Random Conjugation

Antibodies were prepared in 1×DPBS buffer, 2 mM EDTA. TCEP solution from a freshly prepared stock (1.1 mM) in the same buffer was added at 1:2.62, 1:2.26, or 1:1.79 molar equivalents (mAb:TCEP). The solution was mixed thoroughly and incubated at room temperature (21° C.) for 50 minutes. The reduced antibody solution was further diluted with equal volume of 50:50 1×DPBS:propylene glycol, premixed with LP2 (4.71 molar equivalents from a 10 mM DMSO stock solution), to obtain a solution with a final protein concentration of ˜3.5 mg/mL. The conjugation reaction was immediately mixed thoroughly, and conjugation was allowed to proceed at room temperature for a period of approximately 0.5 hour before purification by FPLC using Hitrap desalting (2×5 ml) columns into 25 mM Na Citrate buffer containing 100 mM sucrose, at pH 6.0. The eluate was pooled, filter sterilized (Whatman Puradisc 13, cat. 6791-1302), and stored at 4° C. The purified ADC was analyzed for total protein content (bicinchoninic acid assay, Pierce BCA protocol, catalogue #23225).

### Site-Specific Conjugation

Antibodies were prepared in 1×DPBS buffer, 2 mM EDTA, and added to the linker-payload (5 molar equivalents from a 10 mM DMSO stock solution). The conjugation reaction was immediately mixed thoroughly, and conjugation was allowed to proceed at room temperature for a period of 0.5 hours before purification by FPLC using Hitrap desalting (2×5 mL) columns into 25 mM Na Citrate, 100 mM sucrose, pH 6.0. The eluate was pooled, filter sterilized, and stored at 4° C. The purified ADCs were analyzed for total protein content by BCA.

### Maleimide Conjugation

Decapped RESPECT-L antibodies were prepared at 5 or 7 mg/mL and then incubated with maleimide-linker-payloads at molar ratio of 1:5 in 1×DPBS buffer at room temperature for 30 min. The reaction was then purified by desalting using a HiTrap desalting column or Zeba spin desalting column, eluting with 1×DPBS buffer.

### FACS Analysis

MDA-MB-231 and 4T1 cells were harvested with TrypLE, washed in 2% FBS FACS buffer, and incubated with purified antibodies serially diluted in FACS buffer. Cells were incubated in the dark on ice for 30 minutes, and then washed three times with FACS buffer, followed by incubation with FITC-conjugated secondary antibody. Cells were analyzed using a Guava 8HT.

### BIAcore Analysis

The affinities of mouse anti-human CD73 antibodies were measured using BIAcore T100 and single-cycle kinetics method. The mouse antibodies in culture supernatant were captured on a mouse antibody-capturing BIAcore sensor chip that was prepared using CM5 sensor, mouse antibody capture kit, and amine coupling kit following the manufacturer's instruction. To record binding rates, the increasing concentrations (1 nM, 3 nM, 10 nM, 30 nM, and 100 nM) of antigens, human CD73 and mouse CD73, were injected in series for 80 seconds at a flow rate of 30 μL/min. The dissociations of the antigens were monitored for 30 minutes. Dissociation constants for human CD73 and mouse CD73 were calculated using a 1:1 binding model with BIAcore T100 Evaluation Software.

### Enzymatic Inhibition Assay

MDA-MB-231 and JC-1 cells were used to measure the neutralization of CD73 enzymatic activity. Adherent cells were harvested with TrypLE and then washed in assay buffer (0.5 mM CaCl₂, 120 mM NaCl, 5 mM, KCl 50 mM, Tris-HCl pH 8.0). Cells were then incubated with 10 μg/mL purified mAbs in the dark on ice for 30 minutes and washed three times in assay buffer. The conversion of AMP into adenosine by cell surface CD73 was initiated when the cells were resuspended with 0.5 μM AMP diluted in assay buffer and incubated at 37° C. After 30 minutes, 100 μL/well of supernatant was transferred to a 96-well flat bottom plate for Malachite Green Phosphate detection assay (R+D Systems).

### Internalization Assay

A flow cytometry-based assay was employed to determine if anti-CD73 antibodies can induce internalization of CD73. CD73-expressing cell lines were stained with anti-CD73 mAbs on ice for 30 minutes. Cells were washed and then stained with an Alexa Fluor 488-labeled secondary antibody on ice for 30 minutes. Cells were washed again and resuspended in flow cytometry buffer and incubated on ice or at 37° C. to allow for internalization. At given time points, samples were removed from 37° C. and placed on ice. Cells were then stained with a saturating amount of rabbit anti-Alexa Fluor 488 antibody on ice for 30 minutes. Cells were washed once and run on a flow cytometer. Percent internalization was calculated as quenched sample/non-quenched sample×100.

### ADC Buffer Stability

Buffer stability samples were prepared by diluting ADCs to 1 mg/mL in 25 mM Na Citrate buffer containing 100 mM sucrose, at pH 6.0. The samples were then stored at 37° C. for up to 504 hours. At each time point (T=0, 24, 72, 168, 336, and 504 hours), an aliquot was removed and stored at −80° C. until analysis. Samples were analyzed by HIC-HPLC and SEC-HPLC. DAR was calculated by HIC-HPLC method.

### Thermal Stability Assay

The thermal stability of anti-CD73 Compound 1 ADCs was evaluated by dilution to 1 mg/mL in 1×DPBS buffer and aliquoting to 5 vials at 0.15 mL/vial. The samples were incubated at 37° C. for four time points at T=1, 7, 14, and 21 days. Sample T=0 was prepared as a control sample. At each time point the samples were immediately stored at −80° C. After all time points were collected, samples were thawed at the same time and the DAR was analyzed using HIC-HPLC and LC-MS analysis.

The HIC-HPLC was performed on an Agilent 1260 HPLC system with the TOSOH TSKgel butyl-NPR column (4.6×35 mm). The ADC was loaded to the column for 5 min of 100% buffer A equilibrating, then eluted with 20 minutes of linear gradient from 0-100% buffer B using mobile phases of 1.5 M ammonium sulfate, 25 mM sodium phosphate buffer at pH 7.0 (buffer A), and 25 mM sodium phosphate, 25% IPA (buffer B), followed with 5 min of 100% buffer B washing, at flow rate of 0.6 mL/min.

The SEC-HPLC was performed on an Agilent AdvanceBio SEC 300A column (2.7 μm, 7.8×300 mm) coupled with matched AdvanceBio guard column. Samples were eluted with mobile phase containing 0.1 M sodium phosphate, 0.15 M sodium chloride, 5% IPA at pH 7.4, for total 36 minutes at a flow rate of 0.5 mL/min.

The LC-MS was operated on a Waters Acquity Premier HPLC coupled with SQD2 and 2998 PDA detectors. Samples were reduced with 20 mM DTT at 60° C. for 2 min, then separated by the Waters BioResolve RP mAb Polyphenyl column (4.5×100 mm) using 30 minutes of 25-75% B linear gradient of 0.1% TFA in water (A) and 0.1% TFA in acetonitrile (B), flow rate at 1 mL/min. The linker-payload was conjugated to the unpaired Cysteine 80 at light chain of the antibody and the unpaired Cysteine 118 at the heavy chain of the antibody, and the DAR was calculated based on the integrated peak intensity of identified light chain and heavy chain peaks.

### ADC Plasma Stability

Plasma stability samples were prepared by diluting CD-73 ADCs to 0.4 mg/mL (for Example 4) or 0.2 mg/mL (for Example 7) in mouse plasma (BioIVT, BALB/c mouse plasma prepared using 3.8% sodium citrate as anticoagulant) and aliquoted into 0.1 mL time point samples. Samples were placed into 37° C. incubator and aliquots were removed at 0, 2, 6, 24, 48, 72, 168, and 240 hours (for Example 4) or 0, 4, 24, 72, 168, and 240 hours (for Example 7) and frozen at −80° C. immediately after removal.

### ADC Characterization Methods Analysis of ADC Structure and DAR by LC-MS

Magnetic beads (Dynabeads, Invitrogen, cat 65602) were prepared for immunocapture by washing 3 times with PBS, with collection of the magnetic beads by a DynaMag-2 magnet (Invitrogen, 12321D) after each wash. 40 μg of biotin anti-human Fc (Southern Biotech, 9040-08) per 200 μL stock beads was added to the beads and mixed at room temperature for 1 hour. Beads were washed 3 times with PBS, then brought back to original volume in PBS. 100 μL of plasma sample was added to 200 μL anti-human Fc-immobilized magnetic beads, mixed at room temperature for 2 hours, optionally washed once with PBST, washed 2 times with PBS, and resuspended in 100 μL 2% acetic acid. Beads were mixed at room temperature for 30 minutes. Supernatant was collected and neutralized with 50 μL 1M ammonium bicarbonate. 1 unit of FabRICATOR enzyme (Genovis, A0-FR1) per μg ADC or 10 mL of 10× GlycoBuffer2, 1 mL of PNGase F enzyme (NEB, P0704S) was added to the eluted/neutralized sample and incubated for 1 hour at room temperature or digested with microwave-assisted digestion (Rapid Enzyme Digestion System, Hudson Surface Technologies), 400W, 37° C. for 15 minutes. 4 μL of 100 mM DTT was added to reduce samples and incubated for 20 min at 37° C. Samples were transferred to LC sample vials and analyzed by LC-MS on a Waters Synapt G2 fitted with a Waters Acquity UPLC. DAR was calculated as:

DAR=[(DPA cLC/(DPA cLC+DPA ucLC))+(DPA cHC/(DPA cHC+DPA ucHC))]×2
DPA=deconvoluted peak area
cLC=conjugated light chain
ucLC=unconjugated light chain
cHC=conjugated heavy chain
ucHC=unconjugated heavy chain

### HIC-HPLC

ADCs were subjected to HIC on a TSKgel® Butyl-NPR column (Tosoh Bioscience; 4.6 mm×35 mm i.d.; 2.5 μm particle size) connected to an Agilent 1260 series HPLC at room temperature. Samples were injected (10 μL) at or above 1 mg/mL (at 1 mg/mL for all stability samples). A linear gradient elution was employed starting at 100% mobile phase A/0% mobile phase B, transitioning to 0% mobile phase A/100% mobile phase B over a period of 15 minutes (mobile phase A: 1.5 M ammonium sulfate, 25 mM sodium phosphate at pH 7.0 and mobile phase B: 25% isopropanol (IPA), 25 mM sodium phosphate at pH 7.0), at 0.6 mL/min. Injection of unmodified antibody provided a means of identifying the peak with DAR=0. Antibodies were detected based on absorbance at 280 nm. Peak area was analyzed using Agilent Chemstation software.

DAR was calculated by the following formula:

$$Average DAR = {\sum }_{0}^{8} F ( \% peak area of DAR n * n / 100 )$$

### SEC-HPLC

Antibody-drug conjugate aggregation and fragmentation were analyzed using size exclusion chromatography (SEC) on an Agilent 1200 HPLC system with a DAD (diode array detector). The guard (Agilent AdvanceBio SEC 300 A, 2.7 um, 7.8×50 mm, PL1180-1301) and analytical (Agilent AdvanceBio SEC 300 A, 2.7 um, 7.8×300 mm, PL1180-5301) columns were equilibrated in the mobile phase (0.1 M sodium phosphate, 0.15 M sodium chloride, 5% IPA, pH 7.4) for 3 column volumes. 8 μL of ADC was injected neat and run for 36 minutes at 0.5 mL/min. The ADC was detected at 280 nm with a reference wavelength of 360 nm. Data was analyzed using Agilent Chemstation software (ver. BC.01.07) and reported as % aggregation, % monomer, and % fragmentation based on the peak integration results.

### Analysis of Free Payload by Quantitative LC-MS

Compound 1 and D6-Compound 1 internal standard (IS) powdered reagents were dissolved in LC-MS-grade water. Compound 1 reference standards (standard curve), and QC standards (QC-low, QC-med, and QC-hi) were prepared in mouse plasma, and internal standard for injection was prepared in water. 10 μL of IS was added to each of the standard curve samples, QC controls, and stability samples, and all were prepared using solid-phase extraction (SPE). For SPE, a solid-phase extraction plate (Waters, 186001828BA) was washed with 100 μL water using vacuum. 100 μL of each sample was applied to the plate and drawn through using vacuum. Wells were washed with 5% methanol in water using vacuum. Samples were eluted into a clean collection plate with 25 μL acetonitrile:methanol (60:40). The collection plate was briefly centrifuged to collect all eluted material to the bottom of the collection wells, then 100 μL water was added to each well. Samples were then used for quantitative LC-MS analysis. 10 μL of each eluted sample was injected for LC-MS analysis. Quantitation was done using TargetLynx (Waters).

### In Vivo Efficacy of Anti-CD73-LP ADC in Human Cancer Xenograft Models in Example 5 NCI-H2110 NSCLC Xenograft Model

Female Foxn1 nude mice aged 6 weeks (Jackson Laboratory) were used for Example 5. NCI-H2110 cells (Lot: EB300822-07) were grown in RPMI medium containing 10% HI-FBS. 1×10⁷ NCI-H2110 cells in 1×PBS containing 50% Matrigel were injected subcutaneously in the right flank of the mice at a volume of 100 μL. Tumor growth was monitored via caliper to reach an average of ˜150 mm³, then tumor-bearing mice were randomized into treatment and control groups of 6-8 mice per group.

### MDA-MB-231 BC Xenograft Model

Female FoxnI nude mice aged 6 weeks (Jackson Laboratory) were used for this study. MDA-MB-231 cells (Lot: NB2287-03) were grown in RPMI medium containing 10% HI-FBS. 2×10⁶ MDA-MB-231 cells in 1×PBS containing 50% Matrigel were injected sub-dermally into the 7th/8th m.f.p. at 25 μL/mouse according to the experimental design table using a chilled 27G needle and syringe. The needle was directed towards the animal's spine at an angle near parallel to the surface of the skin and inserted approximately 5 mm. The bevel of the needle should be facing up prior to insertion in the m.f.p. When tumors average 50-100 mm³, tumor-bearing mice were randomized into treatment and control groups of 6-8 mice per group.

### Ca-Ov-3 OV Xenograft Model

Female FoxnI nude mice aged 6 weeks (Jackson Laboratory) were used for this study. Ca-Ov-3-T2 cells (Lot: ELN-20210701) were grown in RPMI medium containing 10% HI-FBS. 1×10⁷ Ca-Ov-3-T2 cells in 1×PBS containing 50% Matrigel were injected subcutaneously in the right flank of the mice at a volume of 100 μL. Tumor growth was monitored via caliper to reach an average of ˜150 mm³, then tumor-bearing mice were randomized into treatment and control groups of 6-8 mice per group.

### HCC-1954 B C Xenograft Model

Female FoxnI nude mice aged 6 weeks (Jackson Laboratory) were used for this study. MDA-MB-231 cells (Lot: NB3225-59) were grown in RPMI medium containing 10% HI-FBS. 2×10⁶ MDA-MB-231 cells in 1×PBS containing 50% Matrigel were injected sub-dermally into the 7th/8th mammary fat pad at 25 μL/mouse according to the experimental design table using a chilled 27G needle and syringe. The needle was directed towards the animal's spine at an angle near parallel to the surface of the skin and inserted approximately 5 mm. The bevel of the needle should be facing up prior to insertion in the mammary fat pad. When tumors average 50-100 mm³, tumor-bearing mice were randomized into treatment and control groups of 6-8 mice per group.

### Mouse Groups and Dosing Regimen

After randomization, the enrolled mice received a single bolus intravenous (IV) injection of 100 μL vehicle or drug. An anti-Staphylococcal enterotoxin B antibody (7909) was included as a negative control. The dosing regimen for each mouse group is shown in Tables 15 and 16.

TABLE 15
Groups and Dosing Regimen for STING-001, -002, -003, and -004 Studies

| | | Dose | | |
|---|---|---|---|---|
| Group | Compound | (mg/kg) | Route | Regimen |
| 1 | Vehicle (PBS) | 0 | IV | Single |
| 2 | 190K12-H4L1-hIgG1-LP2 (DAR4) | 1.0 | IV | Single |
| 3 | 366F5-H1L1-hIgG1-LP2 (DAR4) | 1.0 | IV | Single |
| 4 | 23P11-H1L1-hIgG1-LP2 (DAR4) | 1.0 | IV | Single |
| 5 | 24O6-H1L1-hIgG1-LP2 (DAR4) | 1.0 | IV | Single |
| 6 | 190K12-H4L1-hIgG1-C80-LP2 (DAR2) | 1.0 | IV | Single |
| 7 | 190K12-H4L1-hIgG1-A118C-C80-LP2 (DAR4) | 1.0 | IV | Single |
| 8 | 79G9-hIgG1-LP2 (DAR4) | 1.0 | IV | Single |

TABLE 16
Treatment Groups and Dosing Regimen for
STING-009, -010, -011, and -012 Studies

| | | Dose | | |
|---|---|---|---|---|
| Group | Compound | (mg/kg) | Route | Regimen |
| 1 | Vehicle (PBS) | 0 | IV | Single |
| 2 | 190K12-H4L1-hIgG1-A118C-C80-LP2 (DAR4) | 2.0 | IV | Single |
| 3 | 190K12-H4L1-hIgG1-A118C-C80-LP20 (DAR4) | 2.0 | IV | Single |
| 4 | 190K12-H4L1-hIgG1-A118C-C80-LP16 (DAR4) | 2.0 | IV | Single |
| 5 | 23P11-H1L1-hIgG1-A118C-C80-LP2 (DAR4) | 2.0 | IV | Single |
| 6 | 23P11-H1L1-hIgG1-A118C-C80-LP20 (DAR4) | 2.0 | IV | Single |
| 7 | 23P11-H1L1-hIgG1-A118C-C80-LP16 (DAR4) | 2.0 | IV | Single |
| 8 | 366F5-H1L1-hIgG1-A118C-C80-LP2 (DAR4) | 2.0 | IV | Single |
| 9 | 366F5-H1L1-hIgG1-A118C-C80-LP20 (DAR4) | 2.0 | IV | Single |
| 10 | 366F5-H1L1-hIgG1-A118C-C80-LP16 (DAR4) | 2.0 | IV | Single |
| 11 | 79G9-A1118C-C80-LP2 (DAR4) | 2.0 | IV | Single |

### Tumor Volume and Body Weight Measurements

Tumor volume and body weight were monitored from the day of inoculation until the end point. Tumor volume was measured by digital caliper and was calculated using the formula: W (mm)×L (mm)×D (mm)×π/6.

### Serum or Plasma Collection and Cytokine Analysis

Blood samples were collected five to six hours post-treatment from the retro-orbital sinus/plexus into serum (STING-001, -002, -003, -004) or EDTA-coated plasma (STING-009, -010, -011, -012) collection tubes. Serum samples were allowed to clot at room temperature. All samples were isolated by centrifugation at 1,200×g for 15 min at 4° C. The samples were transferred to clean polypropylene tubes and stored at −80° C. until analysis. Samples from studies STING-001, -002, -003, and -004 were assayed using Codeplex Secrotome Innate Immune-Mouse (IsoPlexis #PANEL-2L12-8) multiplex kits and an IsoLight according to the manufacturer's protocol. Samples from studies STING-009, -010, -011, and -012 were assayed using custom Luminex xMAP multiplex kits designed to detect and quantify the following cytokines: IFN-β, IFN-γ, IL-6, IL-13, IP-10 (CXCL10), MCP-1 (CCL2), MIP-la (CCL3), and MIP-1b (CCL4). All reagents were prepared according to the manufacturer's guidelines. Standards and samples were incubated overnight with magnetic beads and all washing steps were performed using an Agilent automatic plate washer equipped with a magnet insert. Data was collected using a Luminex xMAP Intelliflex and analyzed using EMD Millipore's Belysa software.

### Statistical Analysis

To compare tumor volumes from different groups at pre-specified timepoints (greatest tumor volume differences between the test and control compounds), two-way ANOVA was calculated to test the overall equality of means across all groups and were examined for all pairwise comparisons using Dunnett method. If the p-value of the two-way ANOVA is <0.01 compared to the vehicle group, the anti-CD73-LP ADC efficacy is considered to have significance. In cases where wider tumor volume sample deviation was observed, one-way ANOVA was calculated and the overall quality of mean of selected group was compared with vehicle group using Dunnett method.

### In Vivo Efficacy of Anti-CD73-LP ADC in NCI-H2110 in Example 7 Mouse Groups and Dosing Regimen

BALB/c Slc-nu/nu mice aged 6 weeks (Jackson Laboratory) were used for Example 7.

NSCLC NCI-H2110 cells were grown in RPMI1640 (wako #187-02705) medium containing 10% HI-FBS. 5×10⁶ NCI-H2110 cells in HSBB containing 50% Matrigel were injected subcutaneously in the right flank of the mice at a volume of 100 μL. Tumor growth was monitored via caliper to reach an average of ˜200 mm³, then tumor bearing mice were randomized into treatment and control groups of 5 mice per group. The dosing regimen for each mouse group is shown in Table 17.

TABLE 17
Groups and Dosing Regimen for Example 7

| | | Dose | | |
|---|---|---|---|---|
| Group | Compound | (mg/kg) | Route | Regimen |
| 1 | Vehicle (PBS) | 0 | IV | Single |
| 2 | 190K12-LP2 (DAR4) | 1.0 | IV | Single |
| 3 | 190K12-LP54 (DAR4) | 1.0 | IV | Single |
| 4 | 190K12-LP54 (DAR4) | 2.0 | IV | Single |
| 5 | 190K12-LP55 (DAR4) | 1.0 | IV | Single |
| 6 | 190K12-LP55 (DAR4) | 2.0 | IV | Single |

### Tumor Volume and Body Weight Measurements

Tumor volume and body weights were monitored from the day of inoculation until the end point. The tumor was measured in two dimensions, and the volume was calculated using the following formula: TV (mm³)=½ length (mm)×[width (mm)]² Relative body weight was calculated based on the body weight on the first day of administration.

### Synthesis Methods

Unless otherwise stated, Examples 8 and 9 employed methods that are detailed below.

Organic solutions were dried over anhydrous sodium sulfate or magnesium sulfate. Evaporation of organic solvent was carried out using a rotary evaporator under reduced pressure (0-1000 mbar) with a bath temperature of up to 60° C.

Column chromatography means flash chromatography on silica gel or pre-packed silica gel cartridges (12 g, 24 g, 40 g, etc.). Thin layer chromatography (TLC) was carried out on silica gel plates.

Normal and reverse phase flash column chromatography were carried out using either Teledyne ISCO CombiFlash® systems or Biotage® Flash Columns, used according to the manufacturers' instructions, and obtained from 4700 Superior Street, Lincoln NE 68504, USA or Biotage AB Box 8 751 03 Uppsala Sweden, respectively.

Prep-TLC means preparative TLC plates used in purification. In general, the course of reactions was followed by TLC or liquid chromatography/mass spectroscopy (LC/MS) and reaction times are given for illustration only.

Prep-HPLC means preparative high-performance liquid chromatography, referring to purification using reverse phase HPLC columns listed below and used according to the manufacturer's instructions.

Final products have satisfactory proton nuclear magnetic resonance (NMR) spectra and/or mass spectra data. Preparations were repeated if more material was required.

When given, 1H NMR data are in the form of delta values for major diagnostic protons, given in parts per million (ppm) relative to tetramethylsilane (TMS, 0 ppm) as an internal standard. The coupling constants are reported in the unit of Hertz (Hz). Abbreviations for splitting patterns are as follows: s: singlet; d: doublet; t: triplet; m: multiplet; and brs: broad singlet.

Chemical symbols have their usual meanings. In the event that the nomenclature assigned to a given compound does not correspond to the compound structure depicted herein, the structure will control.

### ABBREVIATIONS

The following abbreviations may be used throughout the examples.
AcOH: Acetic acid
DCM: Dichloromethane
DIEA: N,N-Diisopropylethylamine
DMF: N,N-Dimethylformamide
EDCI: 1-ethyl-3-[3-(dimethylamino)propyl]carbodiimide
ESI: Electrospray ionization
EtOAc: Ethyl acetate
h: hour(s)
HATU: 1-[Bis (dimethylamino)methylene]-1H-1,2,3-triazolo [4,5-b]pyridinium 3-oxidhexafluorophosphate
LiHMDS: Lithium bis(trimethylsilyl)amide
MeCN: Acetonitrile
ODS: octadecylsilyl
rt: room temperature
tert-: tertiary
TFA: Trifluoroacetic acid
THF: Tetrahydrofuran

### Example 2: Generation and In Vitro Characterization of Novel Anti-CD73 Antibodies in Mice

### Generation of Anti-CD73 Antibodies in Mice

Anti-CD73 antibodies were generated in mice immunized with the extracellular domain of human CD73. Given the close homology between human and mouse CD73, CD73-knockout mice were used for immunization. B cells from animals were isolated, grown in 384-well plates, and screened for human CD73 reactivity. High-throughput FACS was used to detect antibodies that bound to CD73-positive MDA-MB-231 cells but did not bind to human CD73-negative Jurkat cells. Antibodies were counter-screened for mouse CD73 reactivity using JC1 cells. The clones showing the greatest reactivity (highest MFI), were cloned and chimerized with human IgG1 and kappa constant domains.

### In Vitro Characterization of Mouse Anti-Human CD73 Antibodies

### FACS Analysis

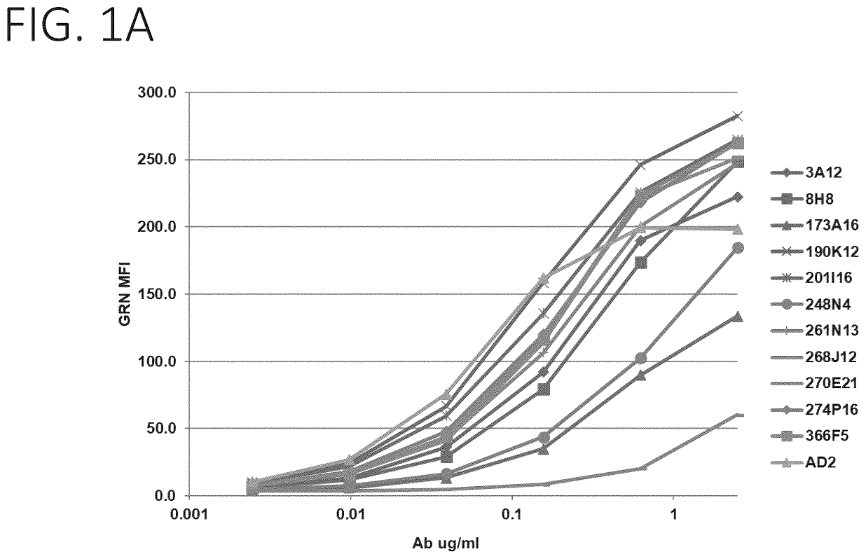

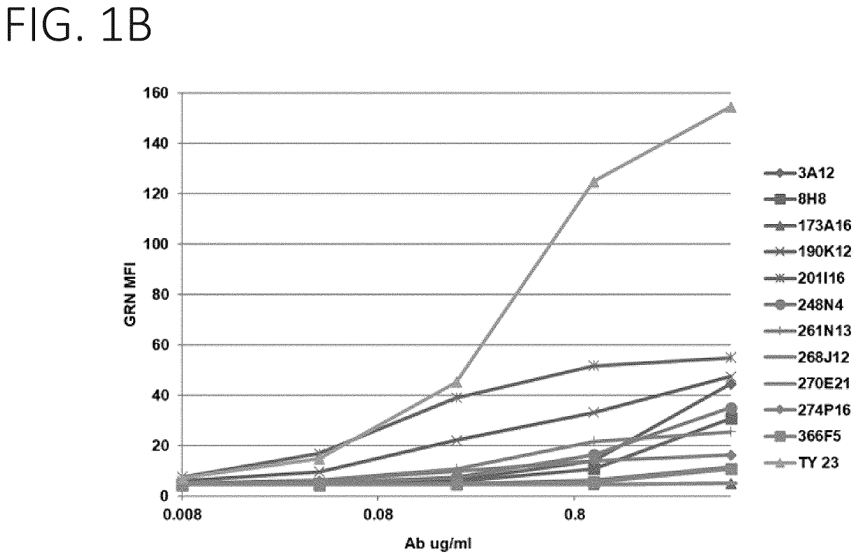

The binding of the recombinantly-expressed antibodies to human or mouse CD73 was measured by FACS titration. For FACS titration, MDA-MB-231 (human CD73-positive) and 4T1 (mouse CD73-positive) cells were incubated with purified antibodies. The clones showing the greatest reactivity are shown in FIG. 1. The positive control anti-human CD73 antibody showed binding to MDA-MB-231 cells, as expected. Clones 190K12, 201I16, 366F5, 270E21, 8H8, 3A12, 261N3, and 274P16 showed high binding to human CD73-expressing MDA-MB-231 cells. Clones 201I16 and 190K12 also demonstrated binding to mouse CD73-expressing 4T1 cells.

### BIAcore Analysis

anti-CD73 antibodies identified by FACS analysis were analyzed by BIAcore to determine their affinities to human and mouse CD73 (Table 18). The association (ka) for all antibodies to human CD73 varied little between the clones. The dissociation (kd) for 190K12, 201I16, 261N13, and 274P16 was similar and 10-25-fold slower than 366F5, 3A12, and 8H8. The latter three clones showed no binding to mouse CD73. Clone 201I16 showed little difference in binding to human and mouse CD73, while 190K12, 261N13, and 274P16 all showed a 25-fold reduction in affinity for mouse compared to human CD73.

TABLE 18
Affinity Analysis of Mouse anti-Human CD73 mAbs

| | human CD73 | | | mouse CD73 | | |
|---|---|---|---|---|---|---|
| clone | ka (1/Ms) | kd (1/s) | KD (M) | ka (1/Ms) | kd (1/s) | KD (M) |
| 190K12 | 5.22E+05 | 1.46E−04 | 2.79E−10 | 5.21E+05 | 3.60E−03 | 6.91E−09 |
| 201I16 | 1.61E+05 | 2.15E−04 | 1.35E−09 | 1.48E+06 | 5.44E−03 | 3.67E−09 |
| 261N13 | 5.06E+05 | 1.43E−04 | 2.83E−10 | 8.82E+05 | 5.61E−03 | 6.36E−09 |
| 274P16 | 7.88E+05 | 4.04E−04 | 5.11E−10 | 5.54E+05 | 7.28E−03 | 1.31E−08 |
| 366F5 | 6.63E+05 | 1.47E−03 | 2.19E−09 | nb | nb | nb |
| 3A12 | 5.31E+05 | 3.52E−03 | 6.49E−09 | nb | nb | nb |
| 8H8 | 3.55E+05 | 2.05E−03 | 5.75E−09 | nb | nb | nb |

nb = no binding observed

### Enzymatic Inhibition of CD73

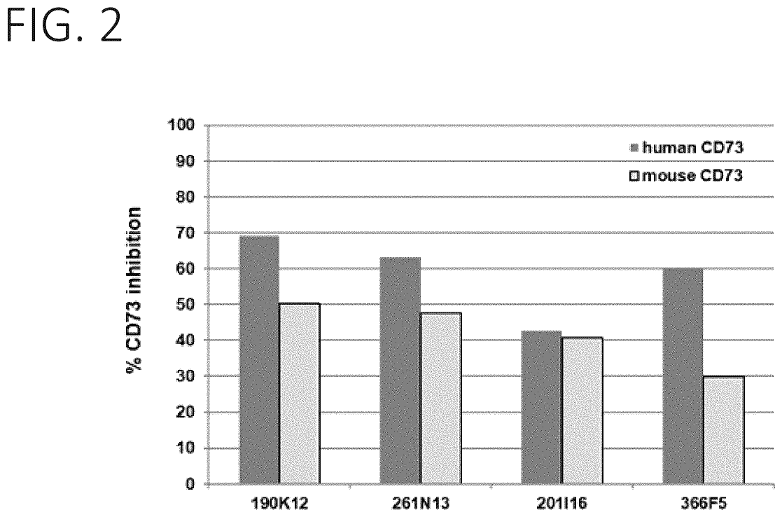

The clones were analyzed for their ability to neutralize CD73-mediated conversion of AMP to adenosine. MDA-MB-231 and JC-1 cells were incubated with mAbs for 30 minutes, washed, and resuspended in 0.5 μM AMP. Following a 30-minute incubation at 37° C., the amount of inorganic phosphate (a product of the conversion of AMP to adenosine) in the supernatant was detected using a Malachite Green Phosphate detection assay (FIG. 2). Clones 190K12 and 261N3 showed high inhibition of both mouse and human CD73 enzymatic activity. Clone 366F5 showed high inhibition of human CD73, but low inhibition of mouse CD73. Clone 201I16 showed low inhibition of human and mouse CD73 enzymatic activity.

Internalization of mAbs

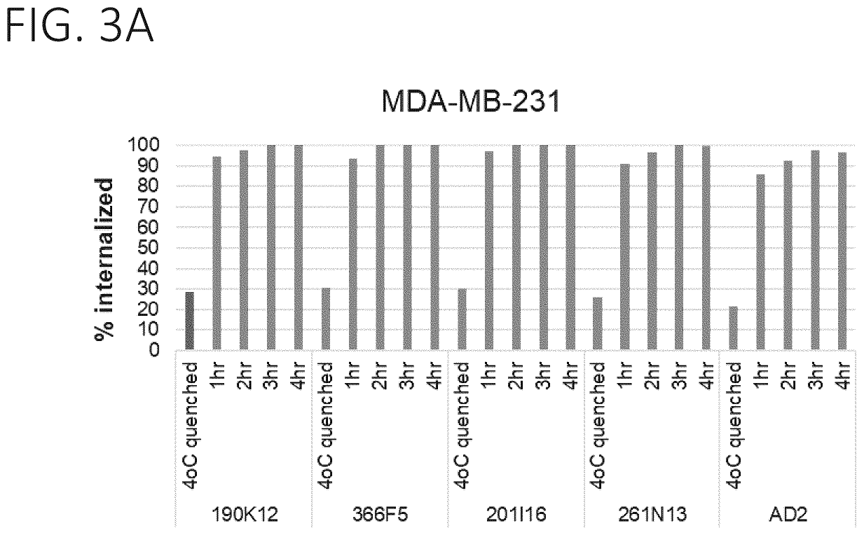

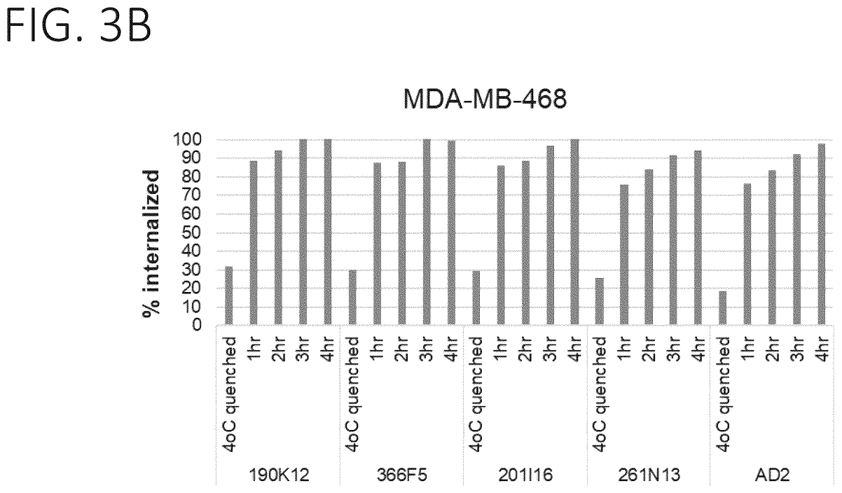

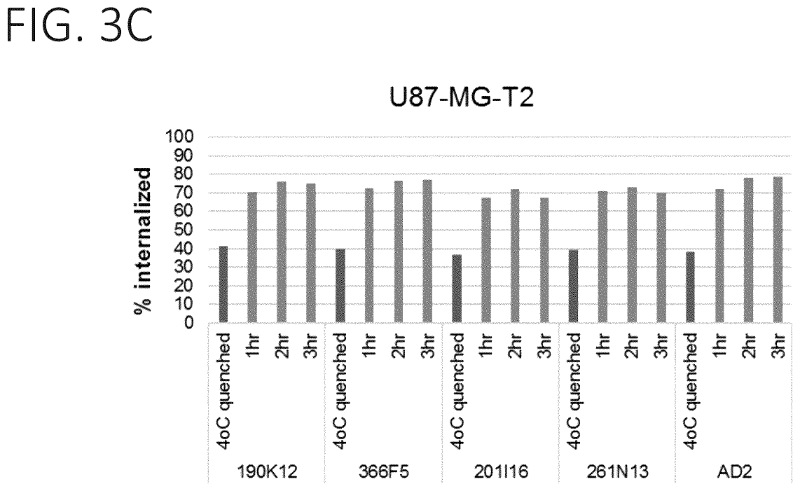

Anti-CD73 clones were analyzed for internalization after binding CD73 on MDA-MB-231, MDA-MB-468, and U87-MG cells. Following binding to cell-surface CD73 by incubating mAbs with cells on ice, antibodies were fluorescently labeled with an Alexafluor488 secondary antibody. Cells were incubated at 37° C. and samples were taken after 0, 1, 2, 3, or 4 hours of incubation and kept on ice. The fluorescence signal from remaining anti-CD73 antibodies on the cell surface was quenched with an anti-Alexafluor488 antibody. The fluorescent signal from internalized anti-CD73 antibodies was measured by FACS. There was little difference in the internalization rate between all clones (FIG. 3). The rate of internalization was highest in MDA-MB-231 cells, and the rate in MDA-MB-468 was similar. U87-MG cells never reached 100% internalization for any clone.

### Mouse Anti-Human Anti-CD73 Antibody Selection

Two mouse-derived anti-CD73 clones were selected for high affinity, enzymatic inhibition, and internalization, but recognizing different epitopes. Clone 190K12 was selected from the clones that bound human and mouse CD73 and clone 366F5 was selected from the clones that bound only human CD73.

### Mouse Anti-Human CD73 Humanization

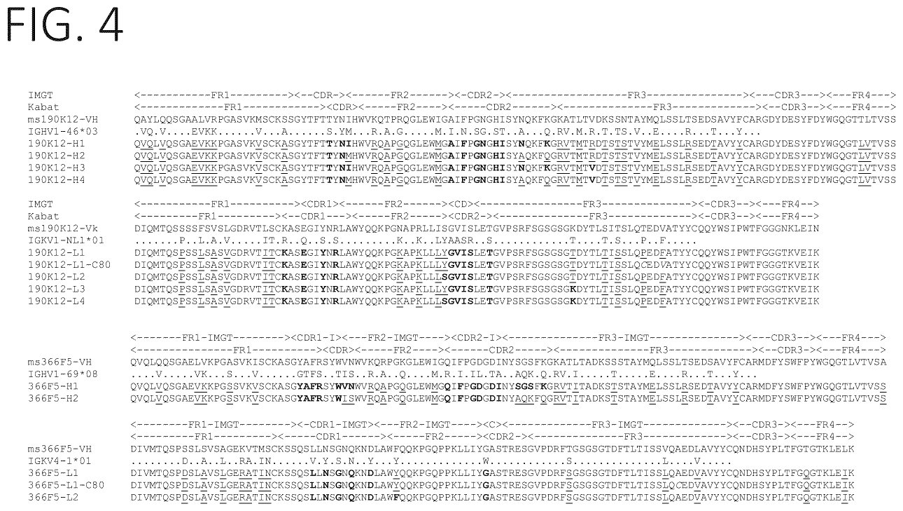

The sequences for the mouse 190K12 and 366F5 mAbs were BLASTed for the closest homology to human germline variable domain protein sequences using http://www.ncbi.nlm.nih.gov/igblast/and http://www.imgt.org/3Dstructure-DB/cgi/DomainGapAlign.cgi (FIG. 4). Mouse framework sequences were replaced with the closest homologous human germline sequences to generate CDR-grafted humanized variants (HCzu1 and LCzu1). IGHV1-46*03 and IGKV1-NL1*01 variable domain families were chosen for 190K12. IGHV1-69*08 and IGKV4-1*01 variable domains were chosen for 366F5. The sequences corresponding to the antigen binding domains as identified by Kabat and Chothia CDRH1, Chothia CDRH2, CDRH3, CDRL1, CDRL2, and CDRL3 were grafted into the framework (FWR) regions of the most homologous human framework to generate H1 and L1 variants.

The mouse and H1-L1 variable domain sequences were used to generate in silico structural models using Biovia Discovery Studio. The theoretical structure of the mouse and humanized models were superimposed, and residues in close proximity to the CDRs were analyzed for potential structural influence on the overall structure of the CDR loops. The residues differing between the mouse and humanized sequences are highlighted in FIG. 4. Most of the differing residues were not located at the dimer interface or were distal to the CDRs. Residues 34, 60, and 64 in 190K12-Vh and 34, 35, 60, 61, 62, and 64 in 366F5-Vh are located in the C-terminal portions of CDRH1 and CDRH2 predicted to be outside of the paratope. These were humanized to generate 190K12-H2 and 366F5-H2, respectively. Residues 71 in 190K12-Vh, 49 and 69 in 190K12-Vx, and 36 in 366F5-Vx were found to be in close proximity to a CDR. These residues were reverted to the mouse residue to generate 190K12-H3, 190K12-L2, 190K12-L3, 190K12-L4, and 366F5-L2, respectively. The super-humanized CDRs and the mouse residue at position 71 in the 190K21 Vh were combined to generate 190K12-H4.

Each heavy chain and each light chain variant for 190K12 or 366F5 were recombinantly expressed in a matrix combination. The affinity for human CD73 was measured for each antibody (Table 19). The KD for all 190K12 Vh-Vx variant combinations were within two-fold. Therefore, the CDRH1 and CDRH2 can be super-humanized without compromising affinity. The humanized residues in 190K12-Vx in proximity to CDRs do not affect CD73 affinity. Similarly, the affinity of the 366F5 humanized variants were within two-fold, indicating CDRH1 and CDRH2 can be super-humanized and residue 36 in Vx can be humanized without affecting affinity to CD73.

TABLE 19
BIAcore Analysis of 190K12 and 366F5 Humanized Variants

| clone | ka (1/Ms) | kd (1/s) | KD (M) |
|---|---|---|---|
| 190K12-H1-L1 | 1.10E+06 | 1.21E−04 | 1.10E−10 |
| 190K12-H1-L2 | 1.67E+06 | 1.01E−04 | 6.05E−11 |
| 190K12-H1-L3 | 1.58E+06 | 8.95E−05 | 5.67E−11 |
| 190K12-H1-L4 | 1.59E+06 | 1.08E−04 | 6.77E−11 |
| 190K12-H2-L1 | 1.92E+06 | 9.06E−05 | 4.72E−11 |
| 190K12-H2-L2 | 2.07E+06 | 1.11E−04 | 5.37E−11 |
| 190K12-H2-L3 | 1.96E+06 | 9.41E−05 | 4.81E−11 |
| 190K12-H2-L4 | 2.11E+06 | 1.11E−04 | 5.25E−11 |
| 190K12-H3-L1 | 1.21E+06 | 6.42E−05 | 5.33E−11 |
| 190K12-H3-L2 | 1.61E+06 | 6.88E−05 | 4.26E−11 |
| 190K12-H3-L3 | 1.30E+06 | 7.95E−05 | 6.13E−11 |
| 190K12-H3-L4 | 1.03E+06 | 1.67E−04 | 1.62E−10 |
| 190K12-H4-L1 | 1.54E+06 | 7.17E−05 | 4.65E−11 |
| 190K12-H4-L2 | 1.16E+06 | 1.25E−04 | 1.08E−10 |
| 190K12-H4-L3 | 1.17E+06 | 1.55E−04 | 1.32E−10 |
| 190K12-H4-L4 | 1.66E+06 | 1.67E−04 | 1.01E−10 |
| 366F5-H1-L1 | 1.97E+06 | 2.27E−04 | 1.16E−10 |
| 366F5-H1-L2 | 1.55E+06 | 1.68E−04 | 1.09E−10 |
| 366F5-H2-L1 | 1.83E+06 | 2.28E−04 | 1.25E−10 |
| 366F5-H2-L2 | 1.61E+06 | 8.70E−05 | 5.40E−11 |

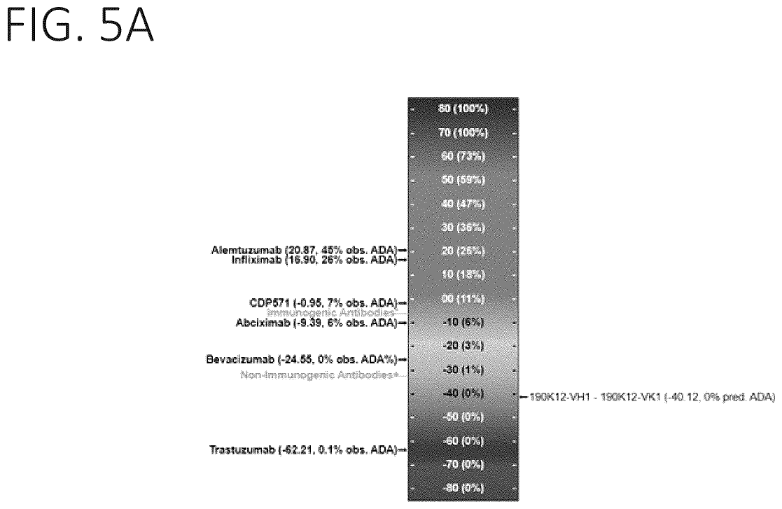

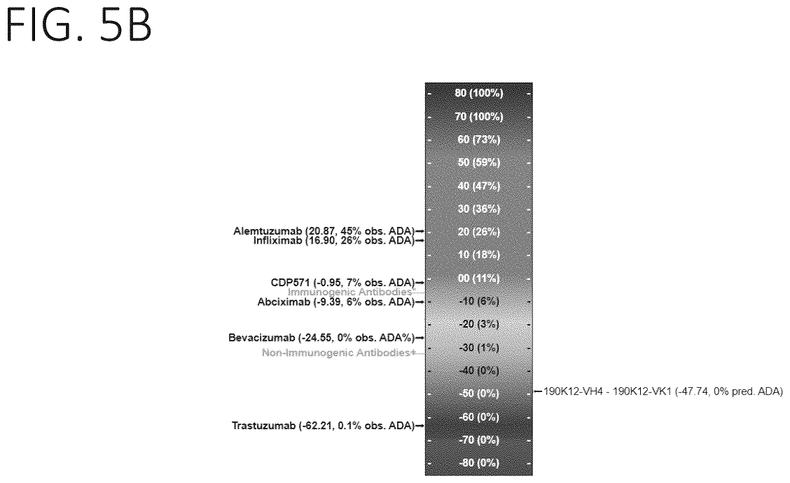

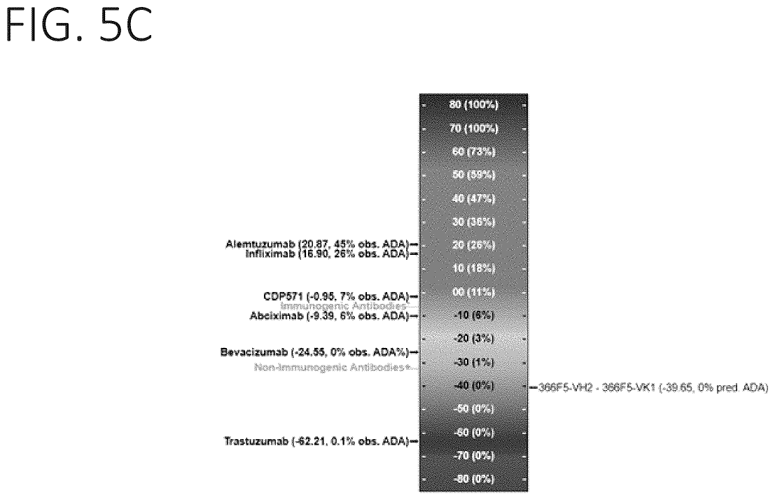

The immunogenicity was predicted in silico for 190K12-H1L1, 190K12-H4L1 (to analyze the effect of a valine at position 71 in Vh), and 366F5-H2L1 using EpiVax's EpiMatrix tool (FIG. 5). The immunogenicity is predicted to be very low for all three antibodies with 190H12-H4L1 predicted to be the lowest. 190K12-H4L1 and 366F5-H1L2 were selected for further characterization.

### Example 3. Generation and In Vitro Characterization of Novel Anti-CD73 Antibodies in Rabbits

### Generation of Anti-CD73 Antibodies in Rabbits

Anti-CD73 antibodies were generated in rabbits immunized with the extracellular domain of human CD73. B cells from animals were isolated, grown in 384-well plates, and screened for human CD73 reactivity. High-throughput FACS was used to detect antibodies that bound to CD73-positive MDA-MB-231 cells but did not bind to human CD73-negative Jurkat cells. Antibodies were counter-screened for rabbit CD73 reactivity using JC1 cells. The clones showing the greatest reactivity (highest MFI), were cloned and chimerized with human IgG1 and kappa constant domains.

### Rabbit Anti-Human CD73 Humanization

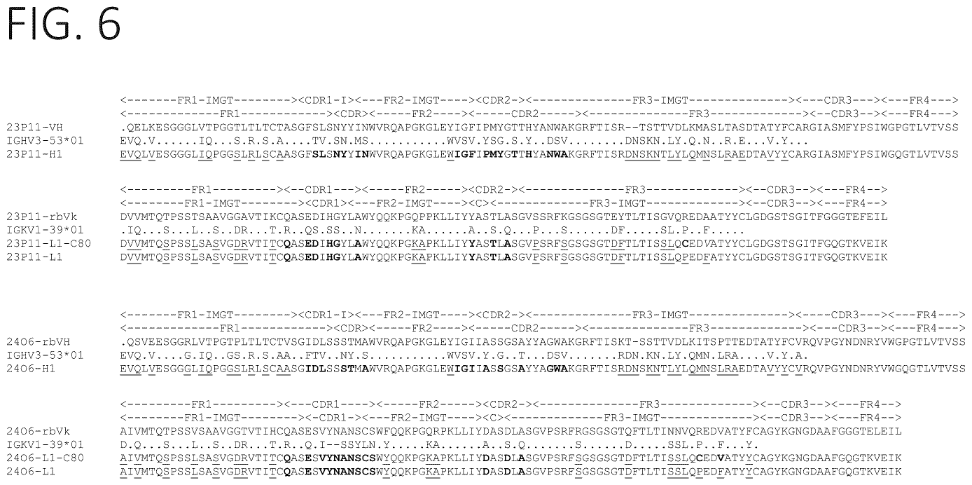

The top clones were humanized by CDR grafting whereby the CDRs from each VH and VL were grafted onto the closest human germline sequence in place of the human CDR sequences. The CDR sequences that were grafted encompassed both the IMGT and Kabat definitions to increase the likelihood the entire paratope was grafted onto the human framework. Residues in the framework that may be critical for retaining affinity and/or stability were retained. These included residues 48-49 in FRH2 and residues 1-3 in FRL1. The cysteine at residue 80 was retained during humanization as a RESPECT-L conjugation site (FIG. 6).

### In Vitro Characterization of Rabbit Anti-Human CD73 Antibodies

### FACS Analysis

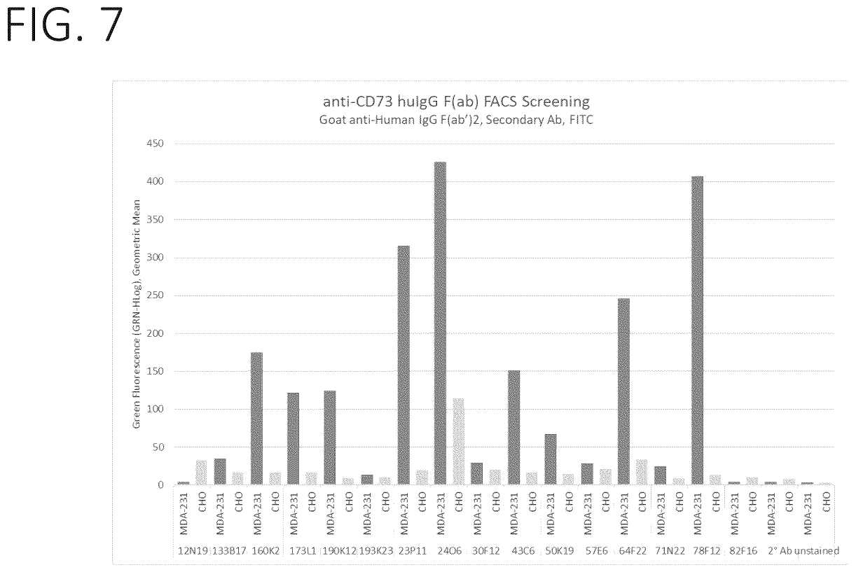

The binding of the recombinantly-expressed antibodies to human CD73 was measured by FACS titration (FIG. 7). For FACS titration, MDA-MB-231 (human CD73-positive) and CHO (CD73-negative) cells were incubated with purified antibodies. The clones showing the greatest reactivity are shown in FIG. 7. The positive control anti-human CD73 antibody 190K12 showed binding to MDA-MB-231 cells, as expected. Clones 23P11, 2406, and 78F12 showed the highest binding to human CD73-expressing MDA-MB-231 cells.

### Enzymatic Inhibition of CD73

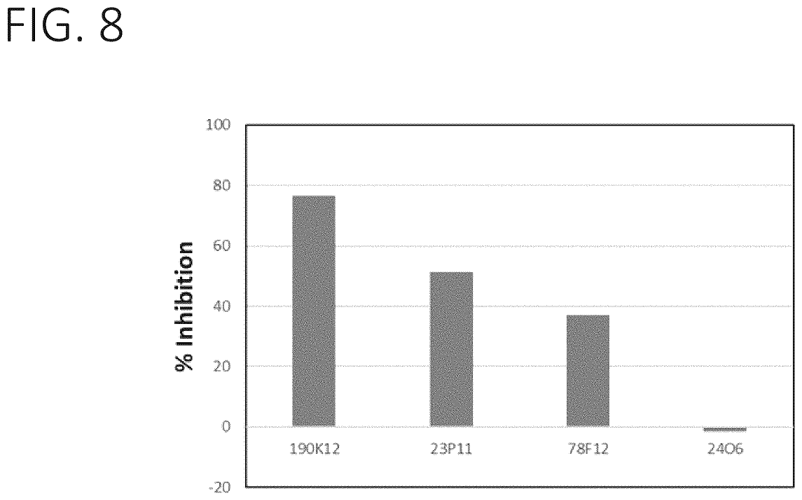

The clones were analyzed for their ability to neutralize CD73-mediated conversion of AMP to adenosine. MDA-MB-231 and JC-1 cells were incubated with mAbs for 30 minutes, washed, and resuspended in 0.5 μM AMP. Following a 30-minute incubation at 37° C., the amount of inorganic phosphate (a product of the conversion of AMP to adenosine) in the supernatant was detected using a Malachite Green Phosphate detection assay (FIG. 8). Clone 190K12 showed high inhibition of human CD73 enzymatic activity as in FIG. 2. Rabbit clones 23P11 and 78F12 showed moderate inhibition of human CD73 enzymatic activity, while clone 24O6 showed no enzymatic inhibition.

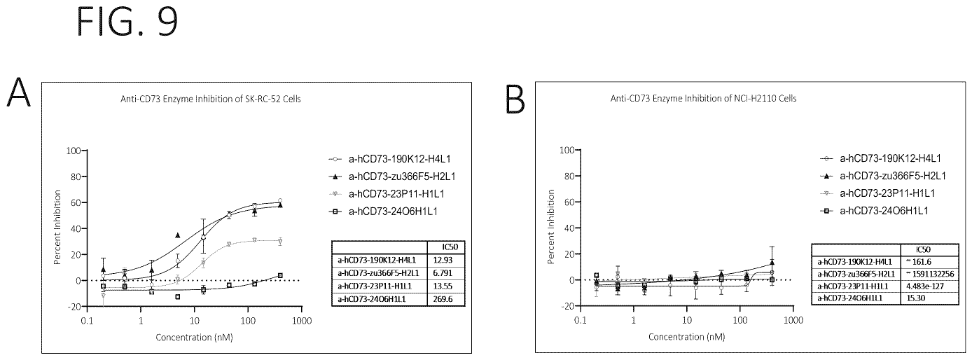

Humanized clones from the mouse and rabbit immunizations were compared in a dose-titration enzymatic inhibition assay. Humanized mAbs were titrated to determine the IC50 of each clone for inhibiting CD73-mediated conversion of AMP to adenosine and inorganic phosphate using SK-RC-52 (FIG. 9A). NCI-H2110 were used as a negative control (FIG. 9B). The mAbs 190K12-H4L1 and zu366F5-H2L1 demonstrated the most enzyme inhibition followed by 23P11-H1L1. No inhibition was observed with 24O6-H1L1.

### BIAcore Analysis

The humanized rabbit anti-CD73 clones 23P11 and 24O6 were analyzed by BIAcore to determine their affinities to human and mouse CD73 (Table 20). Despite strong FACS binding of cell surface-expressed human CD73, 2406 did not bind human or mouse CD73-ECD in the BIAcore assay. Humanized 23P11 showed high affinity for human CD73 at 804 pM, but no binding to mouse CD73.

TABLE 20
BIAcore Analysis of Humanized Rabbit anti-Human CD73 Clones

| Clone | CD73 species | ka (1/Ms) | kd (1/s) | KD (M) |
|---|---|---|---|---|
| 23P11-H1L1 | Human | 1.45E+05 | 1.17E−04 | 8.04E−10 |
| 2406-H1L1 | | nb | nb | nb |
| 23P11-H1L1 | mouse | nb | nb | nb |
| 2406-H1L1 | | nb | nb | nb |

### In Silico Immunogenicity Prediction

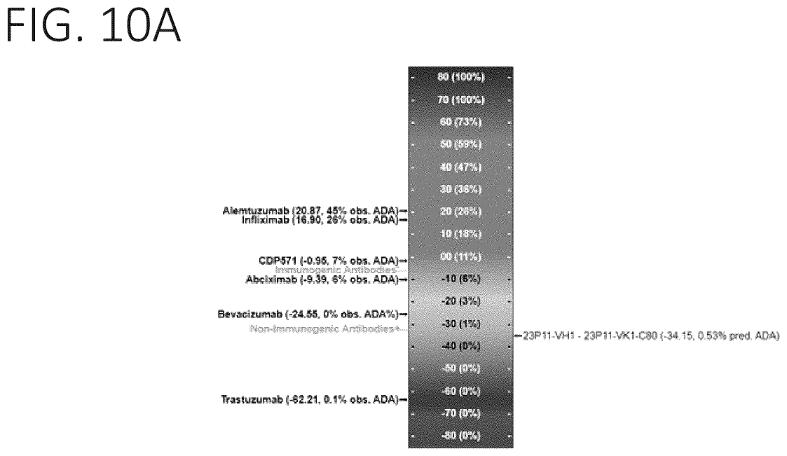

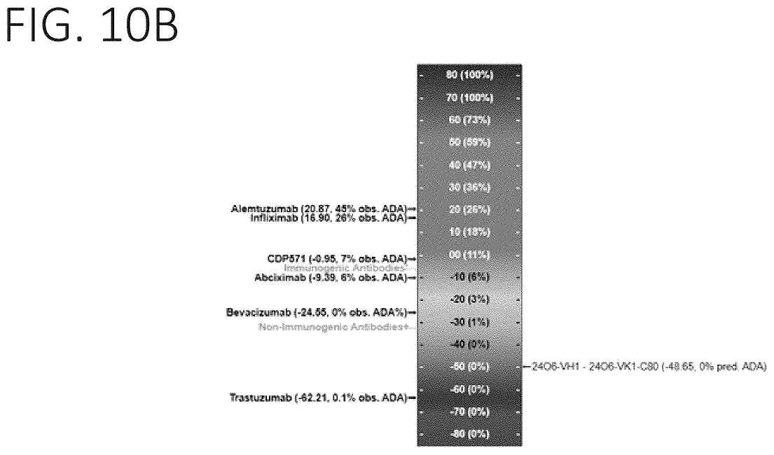

The in silico immunogenicity was predicted for 23P11-H1L1-C80 and 24O6-H1L1-C80 using EpiVax's EpiMarix screening tool (FIG. 10). The immunogenicity and anti-drug antibodies (ADA) were predicted to be very low for both humanized antibodies.

### Example 4. ADC Conjugation and Characterization

### ADC Conjugation

190K12-H4L1, 366F5-H1L1, 23P11-H1L1, and 24O6-H1L1 were conjugated with LP2, LP16, or LP20. Linker payloads were conjugated to the HC-LC or HC-HC interchain cysteines in partially reduced human IgG1 mAbs to an average DAR of ˜4. The ADC product was characterized by HPLC-HIC (high performance liquid chromatography hydrophobic interaction chromatography), SEC, and RP-UPLC-MS (reversed-phase ultra performance liquid chromatography mass spectrometry). The average DAR and drug distribution are derived from interpretation of HIC and LC-MS data (Table 21).

TABLE 21
Randomly conjugated ADC DAR values

| Antibody | LP | DAR (HIC-HPLC) |
|---|---|---|
| 190K12-H4L1-hIgG1 | LP2 | 3.83 |
| 366F5-H1L1-hIgG1 | LP2 | 3.71 |
| 23P11-H1L1-hIgG1 | LP2 | 3.62 |
| 24O6-H1L1-hIgG1 | LP2 | 3.75 |
| 79G9-hIgG1 | LP2 | 4.31 |

The mAbs were also formatted with engineered cysteines at position 80 in the Vx region (C80) to generate site-specific DAR 2 ADCs, or in combination with a cysteine at position 118 in CHI (A118C) to generate site-specific DAR 4 ADCs. The ADC product was characterized by HPLC-HIC, SEC, and RP-UPLC-MS. The average DAR and drug distribution were derived from interpretation of HIC and LC-MS data (Table 22).

TABLE 22
Site-specific ADC DAR values

| Antibody | LP | DAR (HIC-HPLC) |
|---|---|---|
| 190K12-H4L1-hIgG1-A118C-C80 | LP2 | 3.8 |
| 190K12-H4L1-hIgG1-A118C-C80 | LP20 | 4.2 |
| 190K12-H4L1-hIgG1-A118C-C80 | LP16 | 3.98 |
| 190K12-H4L1-hIgG1-C80 | LP2 | 2.01 |
| 366F5-H1L1-hIgG1-A118C-C80 | LP2 | 4.1 |
| 366F5-H1L1-hIgG1-A118C-C80 | LP20 | 4.02 |
| 366F5-H1L1-hIgG1-A118C-C80 | LP16 | 3.47 |
| 23P11-H1L1-hIgG1-A118C-C80 | LP2 | 4.04 |
| 23P11-H1L1-hIgG1-A118C-C80 | LP20 | 4.06 |
| 23P11-H1L1-hIgG1-A118C-C80 | LP16 | 3.72 |
| 190K12-H4L1-14AAS-A118C-C80 | LP2 | 3.97 |
| 79G9-A118C-C80 | LP2 | 4.33 |

### Stability Assays

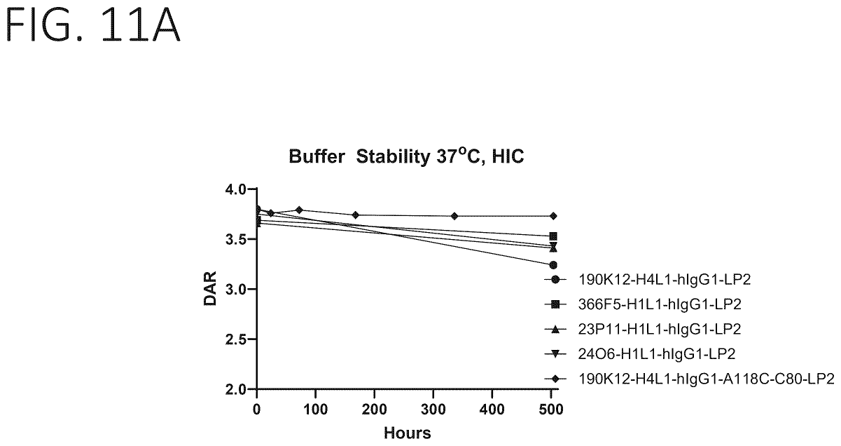

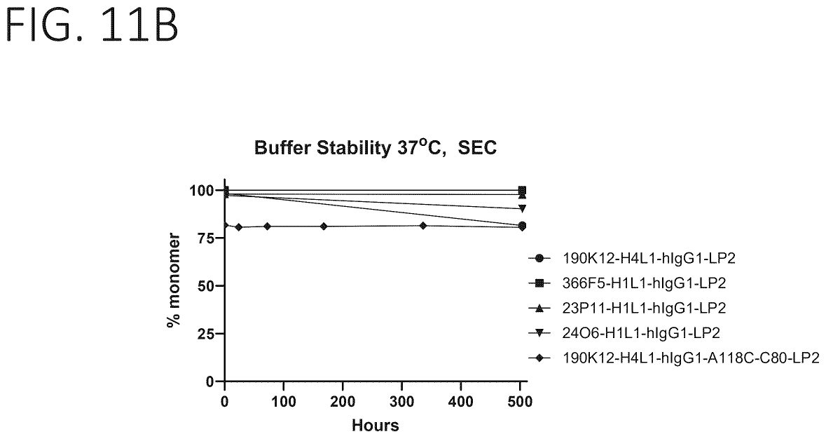

ADCs were evaluated under thermally stressed conditions in buffer and plasma. In buffer, site-specific conjugation was observed to have better retention of DAR by HIC-HPLC (FIG. 11), but lower % monomer by SEC. The ADCs generated from interchain cysteines using mAbs 366F5-H1L1, 23P11-H1L1, and 24O6-H1L1 showed equivalent results, and were observed to have less degradation compared to 190K12-H4L1.

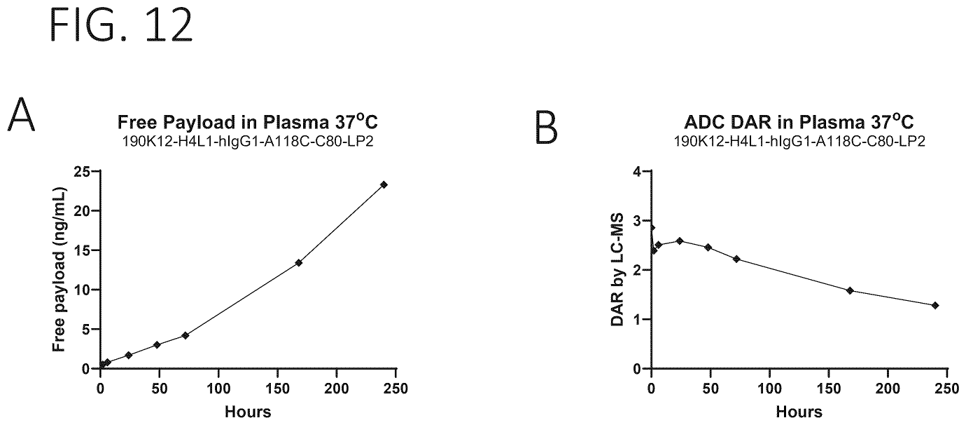

Site-specific ADC stability was then evaluated in plasma. The data shows minimal free payload release and a tolerable loss of DAR for site-specific conjugated 190K12-H4L1-hIgG1 (FIG. 12).

### Example 5. In Vivo Xenograft Models

In order to evaluate the anti-tumor activity of anti-CD73-LP ADCs, four human cancer cell lines were selected based on their level of CD73 expression and the presence of innate STING function shown in Table 23.

TABLE 23
FACS Analysis of Cell Lines Expressing CD73

| Cell Line | NCI-H2110 | HCC1954 | MDA-MB-231 | Ca-OV-3 |
|---|---|---|---|---|
| CD73 (MFI) | 74.9 | 95.3 | 1090.1 | 299 |
| STING Function | − | ++ | + | (+) |

### Anti-Tumor Activity of Anti-CD73-LP ADCs in NSCLC NCI-H2110 Xenograft Mouse Model (STING-001 Study)

### Tumor Growth and Body Weight Change

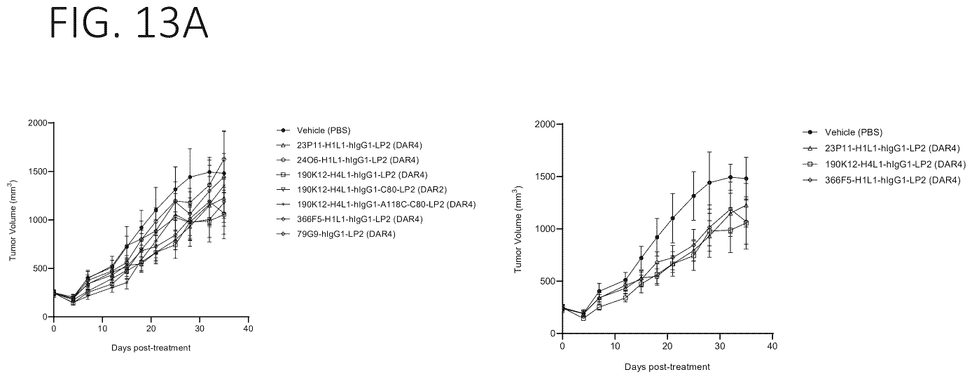

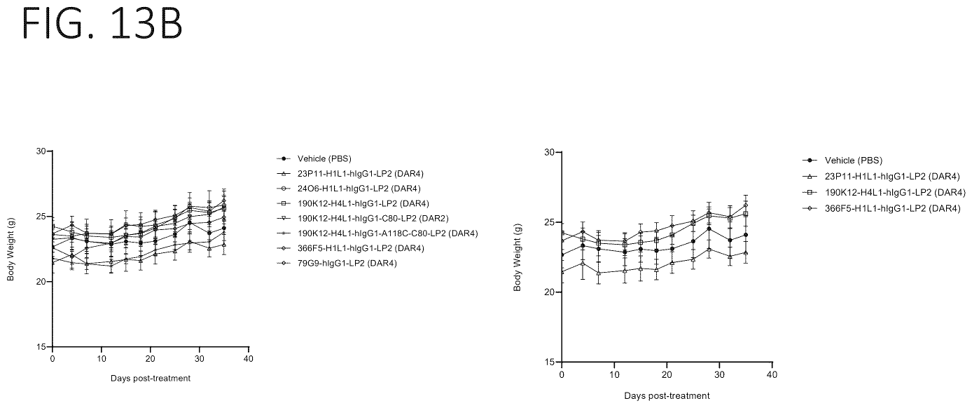

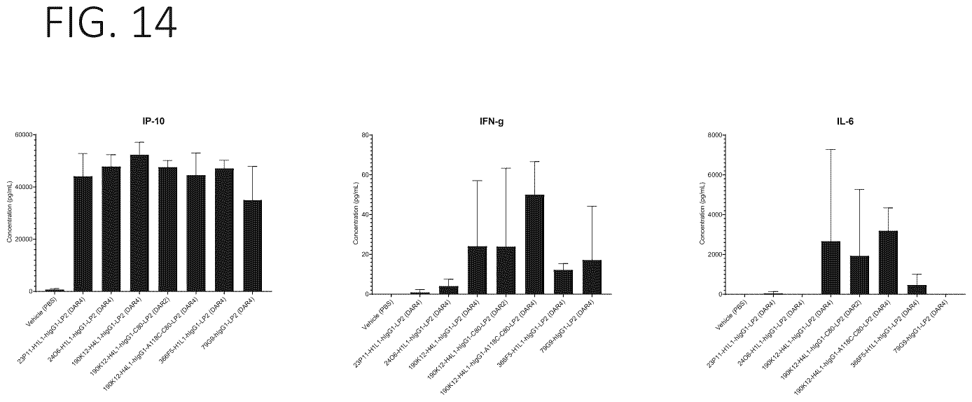

Anti-CD73-LP ADCs were administered as a single IV bolus dose to Foxn1 nude mice bearing NSCLC NCI-H2110 tumors. Average tumor volumes of the treatment groups were compared with control group (vehicle/PBS), and body weight changes were recorded (FIG. 13). While anti-tumor activity was limited at the selected dosage (1 mg/kg), two-way ANOVA analysis demonstrated statistically superior efficacy (P<0.05) in the 23P11-H1L1-hIgG1-LP2 (DAR4), 366F5-H1L1-hIgG1-LP2 (DAR4), and 190K12-H4L1-hIgG1-LP2 (DAR4) treatment groups compared with the vehicle control at day 25. No significance was observed in the remaining treatment groups: 24O6-H1L1-hIgG1-LP2 (DAR4), 190K12-H4L1-hIgG1-A118C-C80-LP2 (DAR4), 190K12-H4L1-hIgG1-C80 (DAR2), and 79G9-hIgG1-LP2 (DAR4). No body weight loss was observed in any of the control and treatment groups.

### Serum Cytokine Analysis

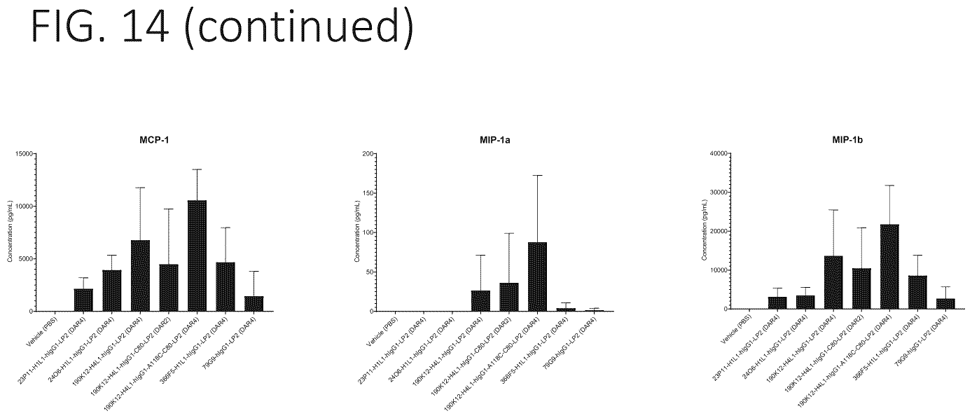

Mouse serum was collected 6 hours post treatment. Cytokine concentrations were measured and quantified using a CodePlex Secrotome Innate Immune (Mouse) multiplex kit (FIG. 14). The analysis demonstrated the presence of STING pathway-specific cytokines six hours post treatment. These data show that treatment with all 190K12-H4L1-LP2 ADCs triggered abundant type-I related cytokine release. The remaining treatment groups demonstrated lower cytokine levels in the NCI-H2110 model. Among the 190K12-H4L1-LP2 ADCs, 190K12-H4L1-hIgG1-LP2 (DAR4) produced higher cytokine levels.

### Anti-Tumor Activity of Anti-CD73-LP ADCs in Breast Cancer (BC) MDA-MB-231 Xenograft Mouse Model (STING-002 Study)

### Tumor Growth and Body Weight Change

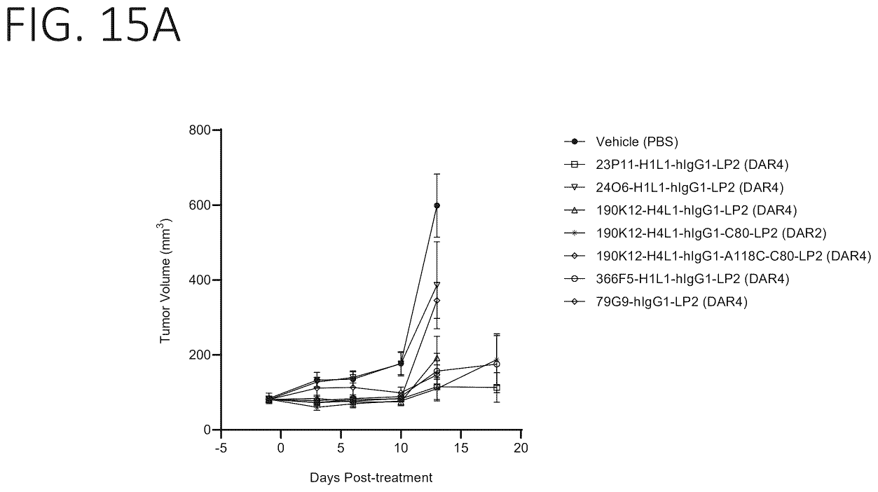

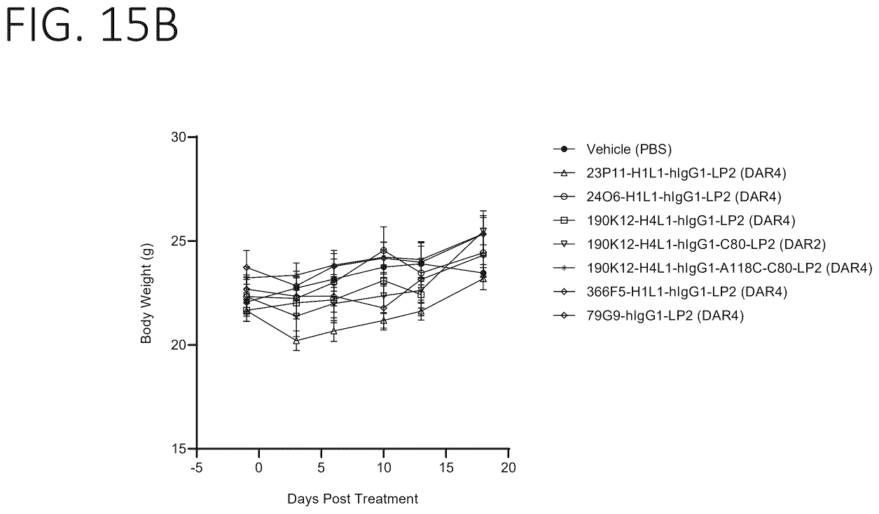

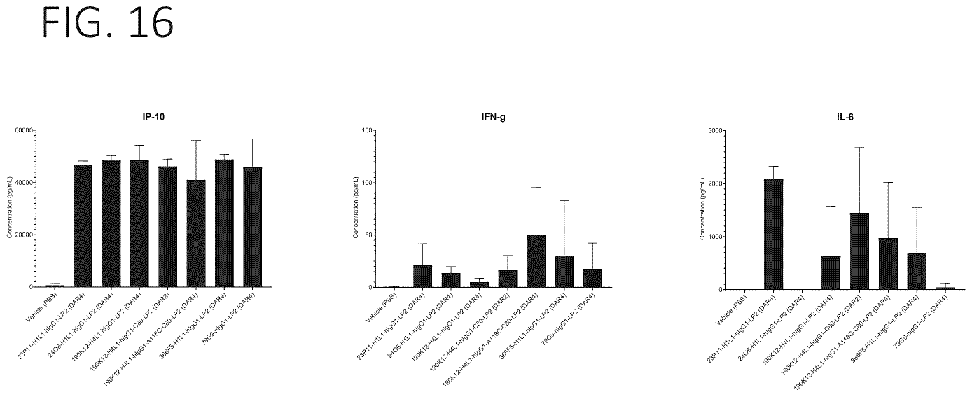

Anti-CD73-LP ADCs were administered as a single bolus IV dose to Foxn1 nude mice bearing BC MDA-MB-231 tumors. Average tumor volumes of the treatment groups were compared with control group (vehicle/PBS) and body weight changes were recorded (FIG. 15). The analysis demonstrated statistically superior anti-tumor activity (P<0.05) in mice treated with 190K12-H4L1-hIgG1-LP2 (DAR4), 190K12-H4L1-hIgG1-C80-LP2 (DAR2), 190K12-H4L1-hIgG1-A118C-C80-LP2 (DAR4), and 23P11-H1L1-hIgG1-LP2 (DAR4) when compared with vehicle control on day 10. No significance was observed in 366F5-H1L1-hIgG1-LP2 (DAR4), 24O6-H1L1-hIgG1-LP2 (DAR4), and 79G9-hIgG1-LP2 (DAR4) treatment groups. No body weight loss was observed in any of the control and treatment groups.

### Serum Cytokine Analysis

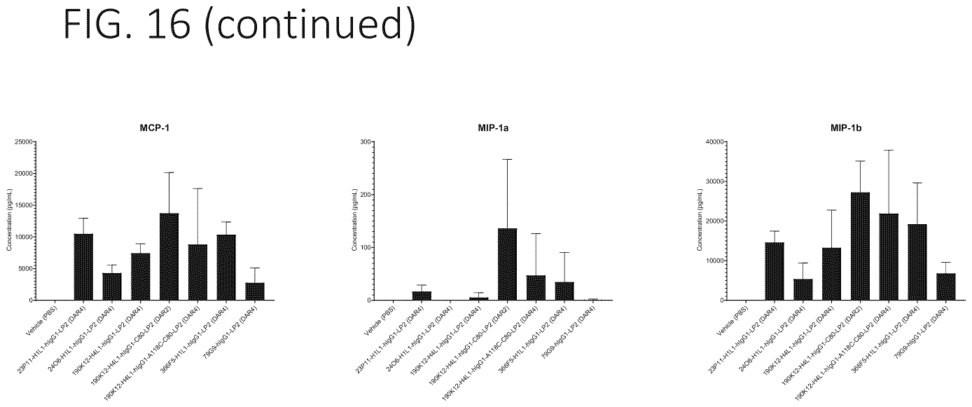

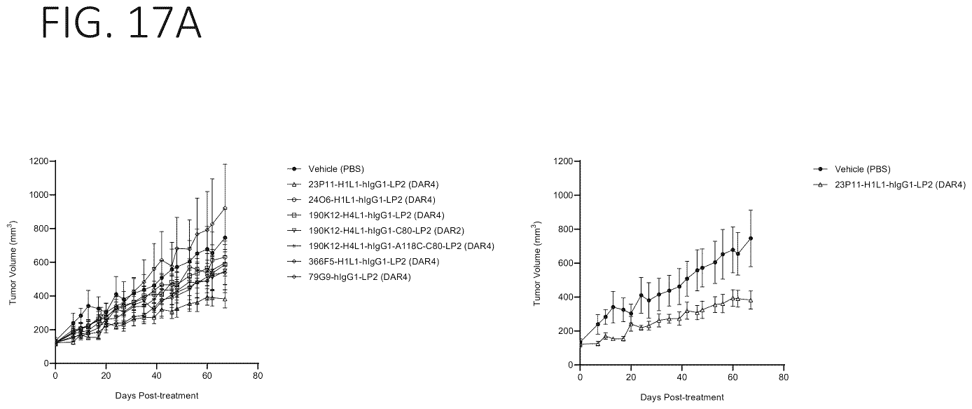

Mouse serum was collected 6 hours post treatment. Cytokine concentrations were measured and quantified using a CodePlex Secrotome Innate Immune (Mouse) multiplex kit (FIG. 16). The analysis demonstrated the presence of STING pathway-specific cytokines six hours post treatment. These data show that treatment with either of the DAR4 190K12-H4L1-LP2 ADCs or with 23P11-H1L1-hIgG1-LP2 (DAR4) leads to abundant type I-related cytokine release. The remaining treatment groups showed lower levels of cytokines in the MDA-MB-231 model. In this study, 23P11-H1L1-hIgG1-LP2 (DAR4) elicited high amounts of IL-6 release. This treatment group also showed durable anti-tumor activity.

### Anti-Tumor Activity of Anti-CD73-LP ADCs in Ovarian Cancer (OV) Ca-OV-3-T2 Xenograft Mouse Model (STING-003 Study)

### Tumor Growth and Body Weight Change

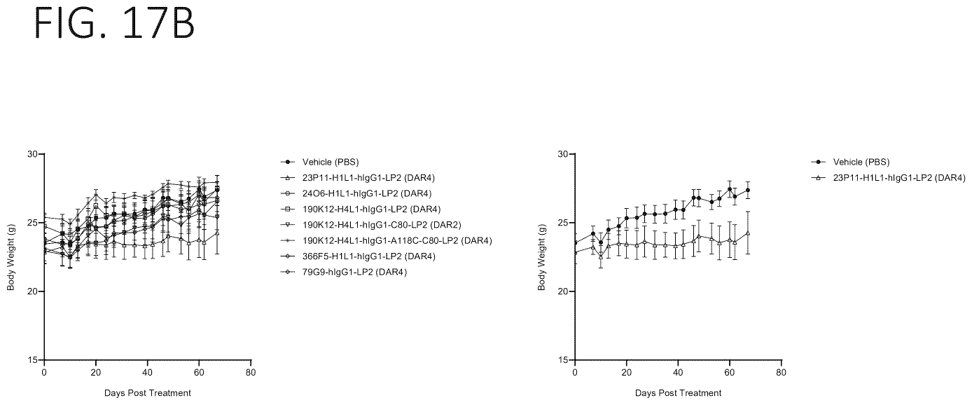

Anti-CD73-LP ADCs were administered as a single IV bolus dose to FoxnI nude mice bearing OV Ca-OV-3-T2 tumors. Average tumor volumes of the treatment groups were compared with control group (vehicle/PBS) and body weight changes were recorded (FIG. 17). The analysis demonstrated no statistically significant anti-tumor activity (P<0.05) by anti-CD73-LP2 treatment compared with vehicle control on day 56. There was limited statistical significance (p=0.084) observed in 23P11-H1L1-hIgG1-LP2 (DAR4)-treated mice at this timepoint. However, there were deviations in tumor volume observed at this dosage (1 mg/kg). No body weight loss was observed in any of the control and treatment groups.

### Serum Cytokine Analysis

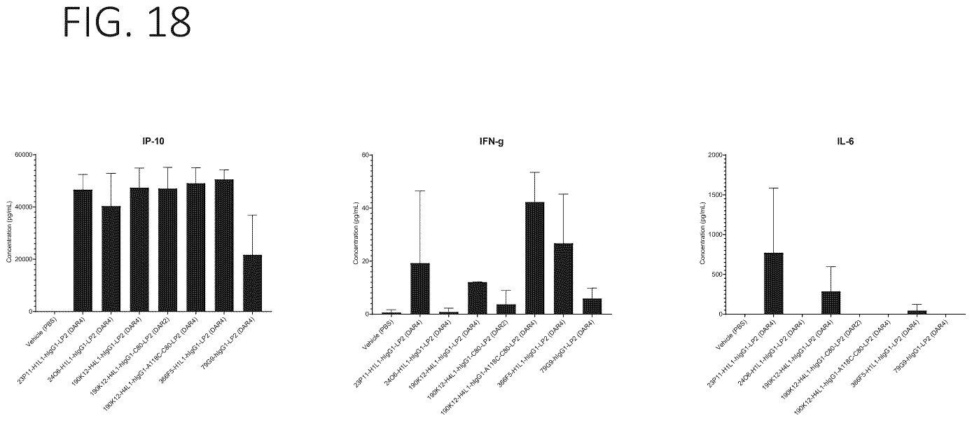

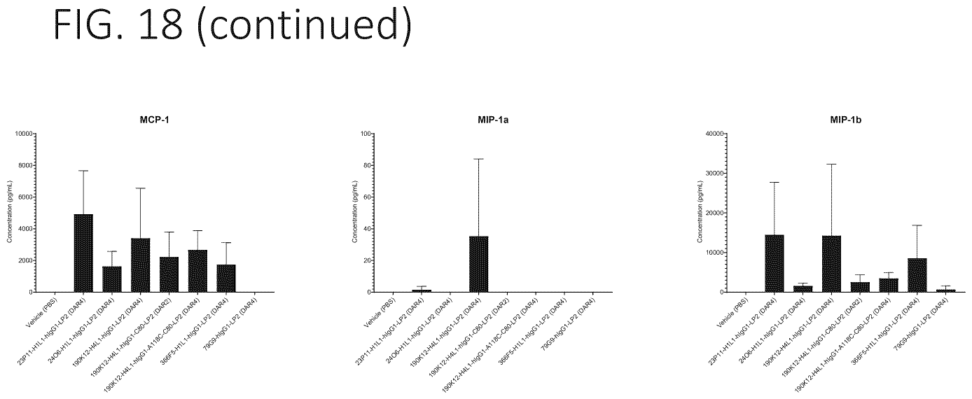

Mouse serum was collected 6 hours post treatment. Cytokine concentrations were measured and quantified using a CodePlex Secrotome Innate Immune (Mouse) multiplex kit (FIG. 18). The analysis demonstrated the presence of STING pathway-specific cytokines six hours post treatment. These data show that treatment with either of the DAR4 190K12-H4L1-LP2 ADCs or with 23P11-H1L1-hIgG1-LP2 (DAR4) cause abundant type I-related cytokine release. Lower cytokine levels were observed in the remaining treatment groups in the Ca-OV-3 model. In this study, 23P11-H1L1-hIgG1-LP2 (DAR4) induced the highest IL-6 cytokine release and treatment with 190K12-H4L1-hIgG1-LP2 (DAR4) showed the highest concentrations of IFN-γ and MIP-la.

### Anti-Tumor Activity of Anti-CD73-LP ADCs in BC HCC1954 Xenograft Mouse Model (STING-004 Study)

### Tumor Growth and Body Weight Change

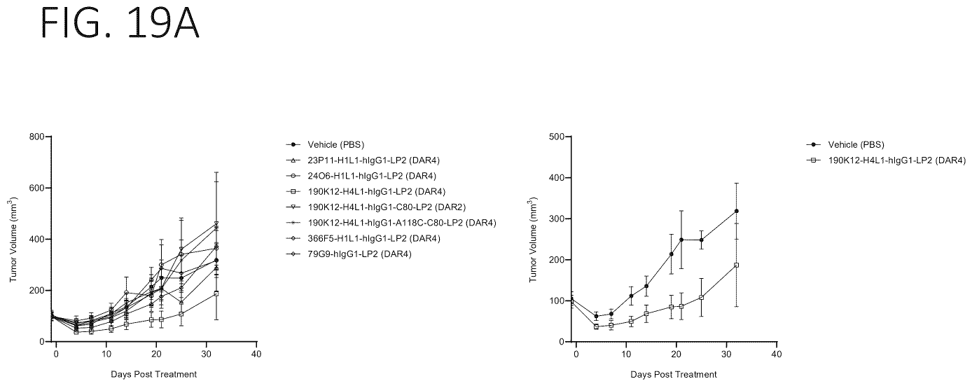

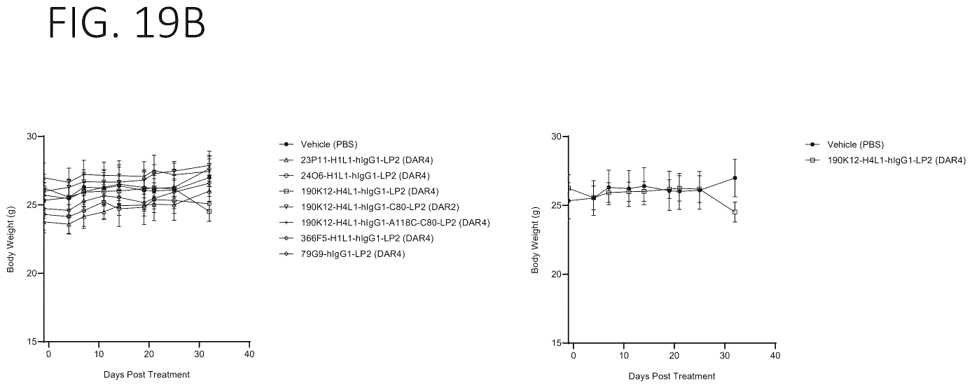

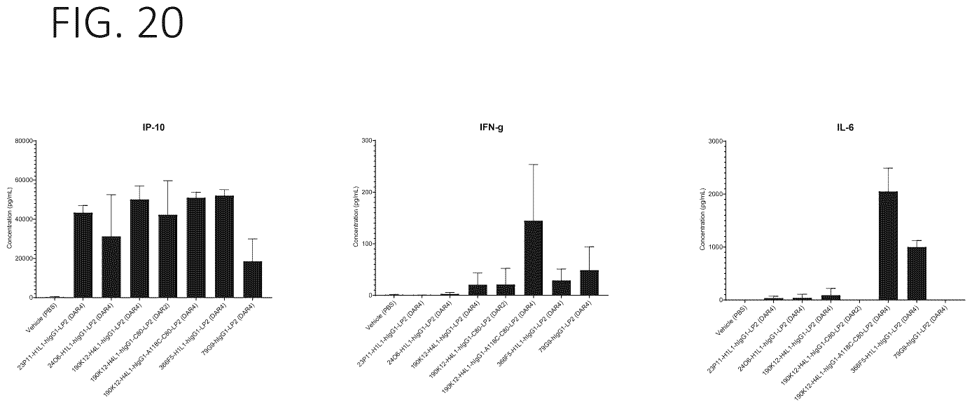

Anti-CD73-LP ADCs were administered as a single bolus IV dose to Foxn1 nude mice bearing BC HCC1954 tumors. Average tumor volumes of the treatment groups were compared with control group (vehicle/PBS) and body weight changes were recorded (FIG. 19). The analysis did not demonstrate statistically superior anti-tumor activity by any of the anti-CD73-LP ADCs as determined by two-way ANOVA. However, anti-tumor activity in mice treated with 190K12-H4L1-hIgG1-LP2 (DAR4) was statistically significant by one-way ANOVA (P=0.012) on day 32 post dose. No body weight loss was observed in any of the control and treatment groups.

### Serum Cytokine Analysis

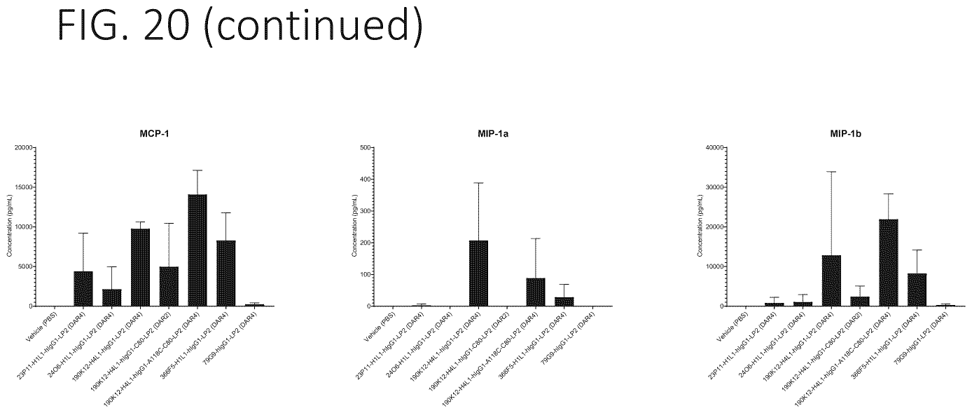

Mouse serum was collected 6 hours post treatment. Cytokine concentrations were measured and quantified using a CodePlex Secrotome Innate Immune (Mouse) multiplex kit (FIG. 20). The analysis demonstrated the presence of STING pathway-specific cytokines six hours post treatment. These data show that either of the DAR4 190K12-H4L1-LP2 ADCs cause abundant type I-related pro-inflammatory cytokine release, while 190K12-H4L1-hIgG1-C80-LP2, 366F5-H1L1-hIgG1-LP2 (DAR4), 23P11-H1L1-hIgG1-LP2 (DAR4), 24O6-H1L1-hIgG1-LP2 (DAR4), and 79G9-hIgG1-LP2 (DAR4) induce lower cytokine release in the HCC1954 model. Treatment with 190K12-H4L1-hIgG1-LP2 (DAR4) caused comparatively high levels of MIP1α and MIP1β release, which seemed to correlate with better anti-tumor activity.

### Anti-Tumor Activity of Anti-CD73-LP ADCs in NSCLC NCI-H2110 Xenograft Mouse Model (STING-009 Study)

### Tumor Growth and Body Weight Change

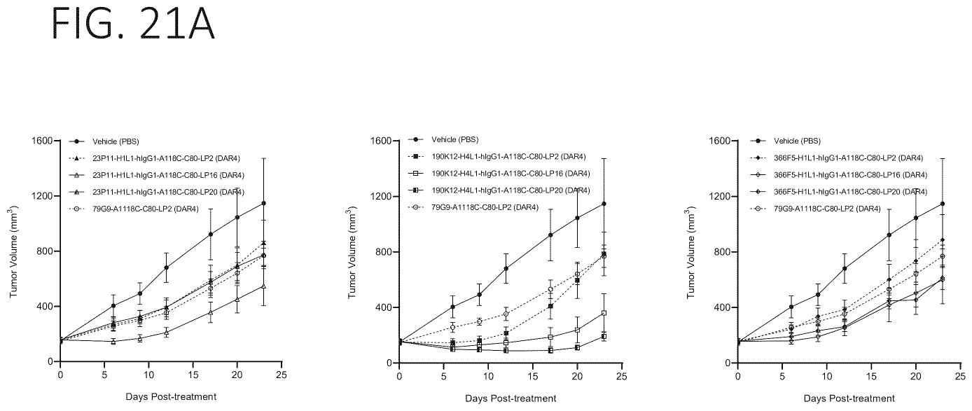

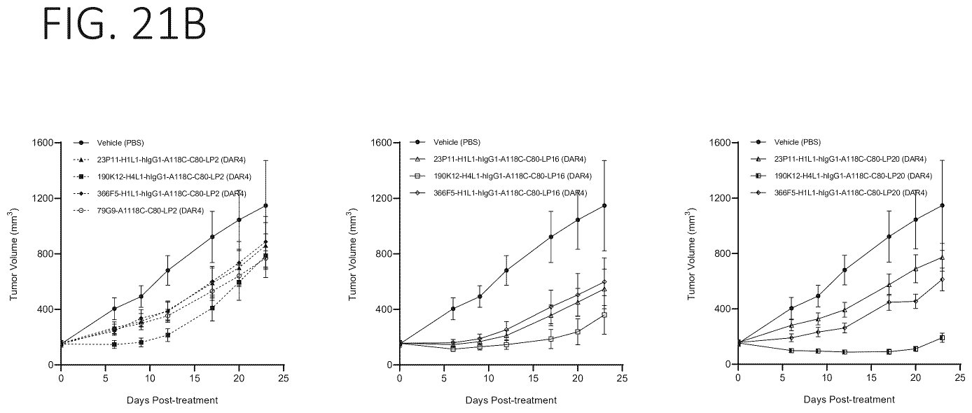

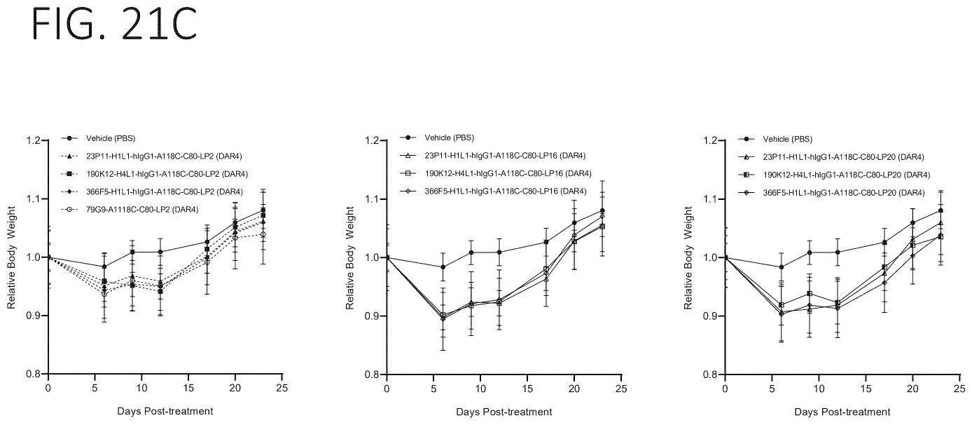

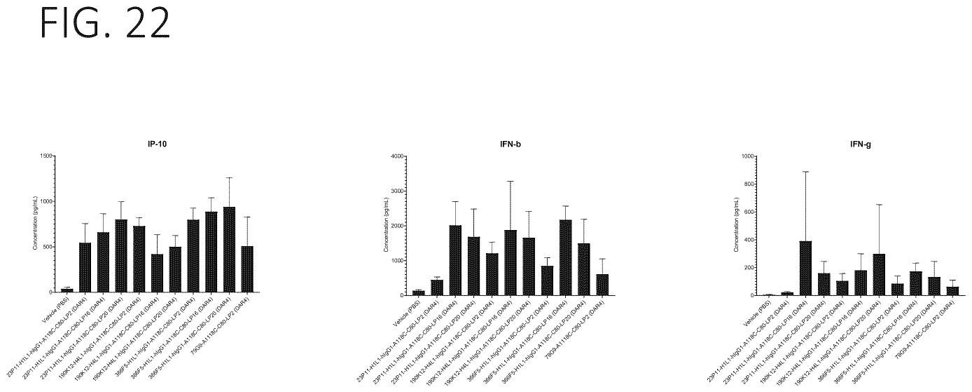

Anti-CD73-LP ADCs were administered as a single IV bolus dose to Foxn1 nude mice bearing NSCLC NCI-H2110 tumors. Average tumor volumes of the treatment groups were compared with control group (vehicle/PBS) and body weight changes were recorded (FIG. 21). This study showed that treatment with LP16 and LP20 ADCs leads to superior anti-tumor activity compared to LP2 ADCs. It also revealed that targeting treatment with anti-CD73 clone 190K12-H4L1 results in better efficacy than clones 23P11-H1L1 and 366F5-H1L1. At this dosage (2 mg/kg), 5-10% body weight loss was observed in all treatment groups. These mice fully recovered their lost body weight by day 20.

### Plasma Cytokine Analysis

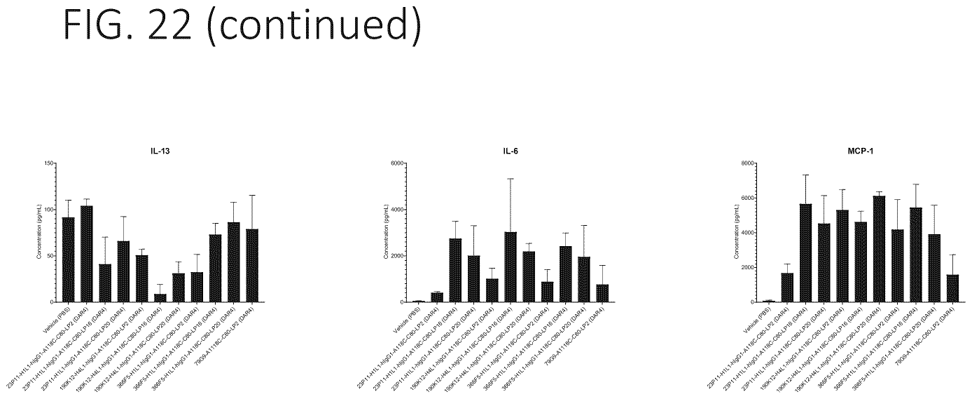

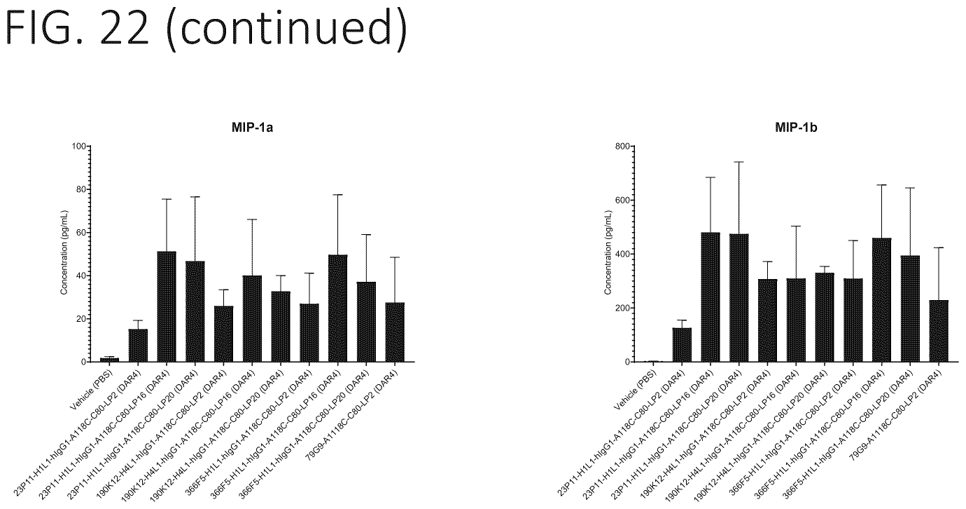

Mouse plasma was collected 6 hours post treatment. Cytokine concentrations were measured and quantified using a Luminex xMAP multiplex kit (FIG. 22). The analysis demonstrated the presence of STING pathway-specific cytokines six hours post treatment. These data show that LP16 and LP20 ADC-treated mice produced cytokines [INF-β, INF-γ, IL-6, MCP1, MIP1α] at higher levels than LP2 ADC-treated mice regardless of which anti-CD73 clone (190K12-H4L1, 366F5-H1L1, or 23P11-H1L1) was incorporated in the ADC, which seemed to correlate with better anti-tumor activity in the NCI-H2110 model.

### Anti-Tumor Activity of Anti-CD73-LP ADCs in BC MDA-MB-231 Xenograft Mouse Model (STING-010 Study)

### Tumor Growth and Body Weight

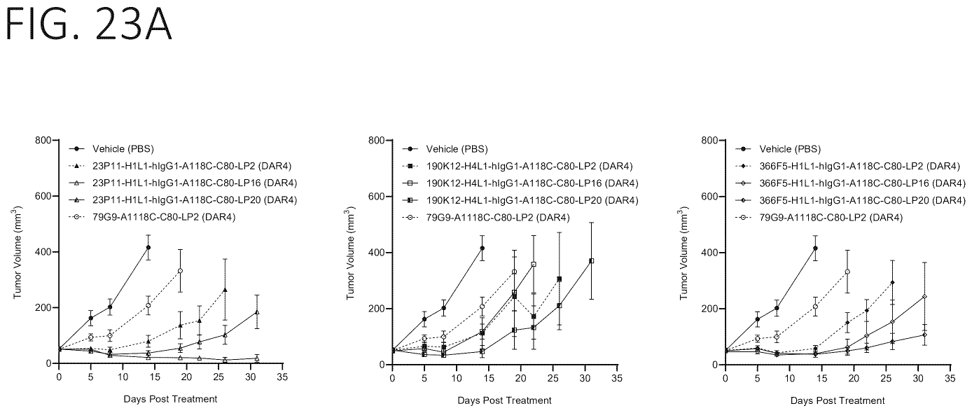

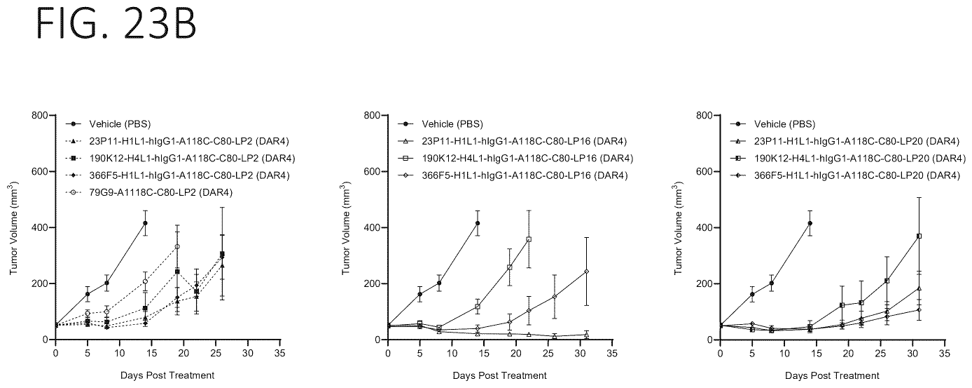

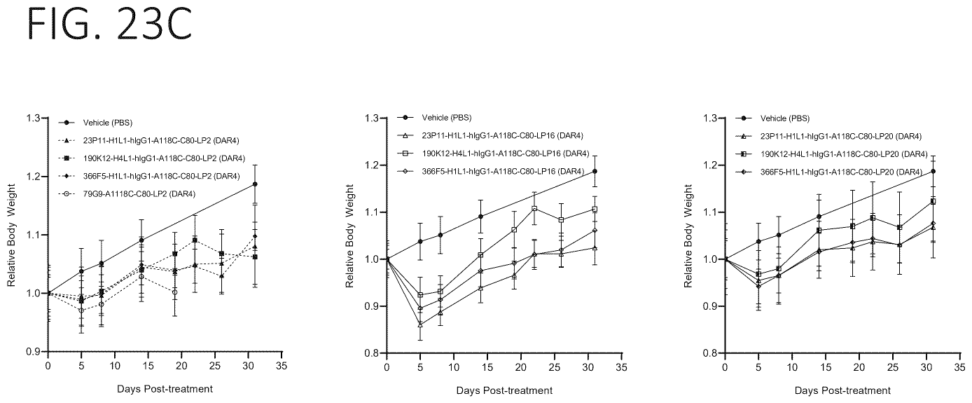

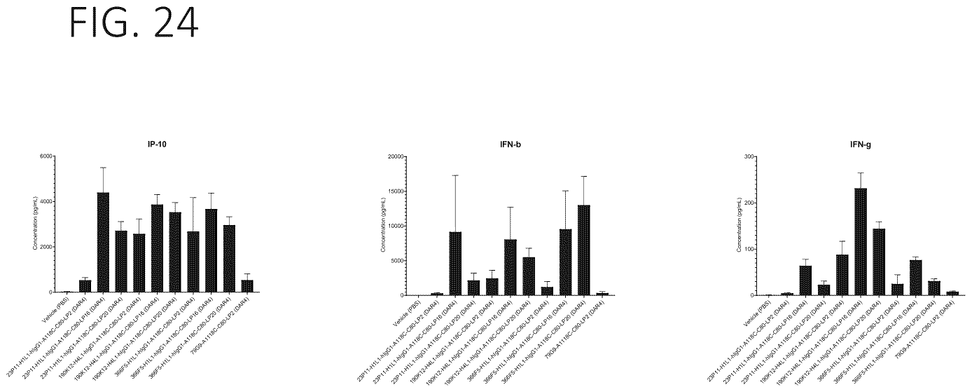

Anti-CD73-LP ADCs were administered as a single bolus IV dose to Foxn1 nude mice bearing BC MDA-MB-231 tumors. Average tumor volumes of the treatment groups were compared with control group (vehicle/PBS) and body weight changes were recorded (FIG. 23). This study showed that treatment with LP16 and LP20 ADCs leads to superior anti-tumor activity compared to LP2 ADCs. In this model, clones 23P11-H1L1 and 366F5-H1L1 ADCs showed more durable anti-tumor responses than 190K12-H4L1. The best efficacy was observed in mice treated with 23P11-H1L1-hIgG1-A118C-C80-LP16. Acute body weight loss of 5-10% was observed in most treatment groups.

### Plasma Cytokine Analysis

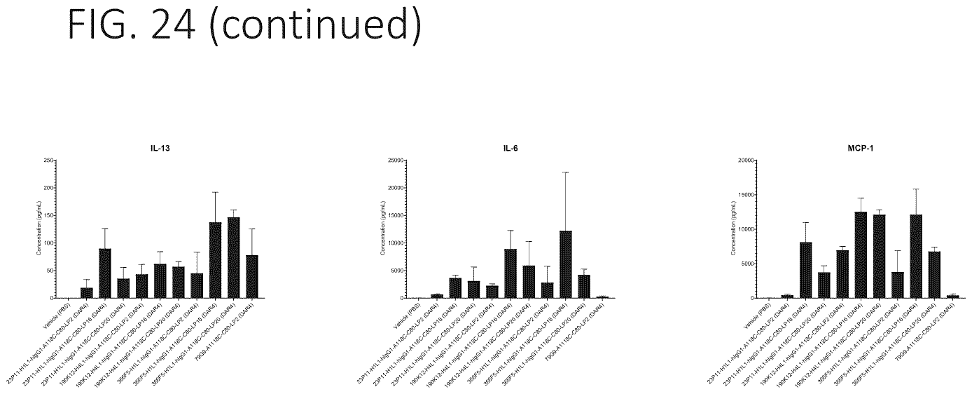

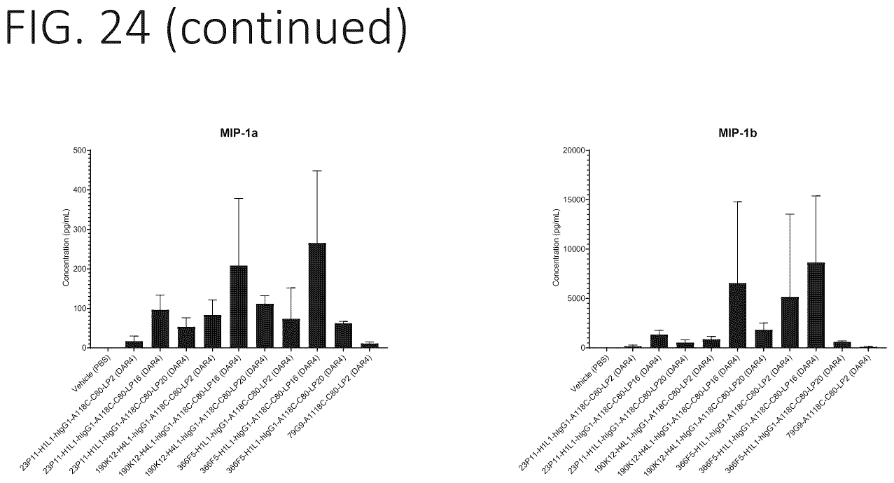

Mouse plasma was collected 6 hours post treatment. Cytokine concentrations were measured and quantified using a Luminex xMAP multiplex kit (FIG. 24). These data show that LP16 and LP20 ADC-treated mice produced cytokines, except MIP1P, at higher levels than LP2 ADC-treated mice, regardless of which anti-CD73 clone was incorporated in the ADC. These ADCs tend to correlate with better anti-tumor activity in this MDA-MB-231 model.

### Anti-Tumor Activity of Anti-CD73-LP ADCs in OV Ca-OV-3-T2 Xenograft Mouse Model (STING-011

### Tumor Growth and Body Weight Change

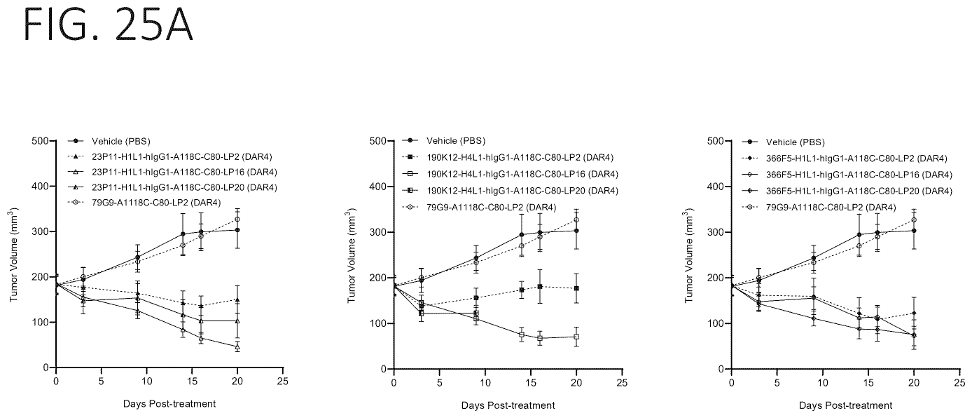

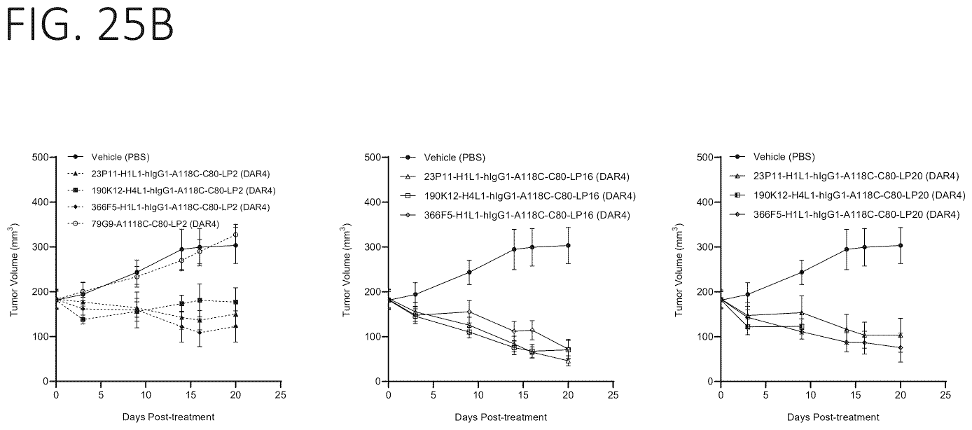

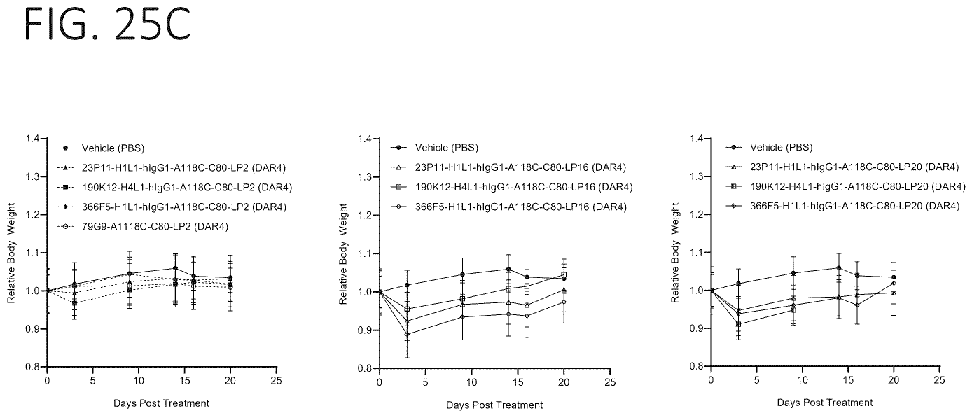

Anti-CD73-LP ADCs were administered as a single IV bolus dose to Foxn1 nude mice bearing OV Ca-OV-3-T2 tumors. Average tumor volumes of the treatment groups were compared with control group (vehicle/PBS) and body weight changes were recorded (FIG. 25). This study showed that treatment with LP16 and LP20 ADCs leads to slightly superior anti-tumor activity compared to LP2 ADCs. In this model, all three anti-CD73 clones showed similar anti-tumor activity. No body weight loss was observed in the LP2 ADCs. Acute body weight loss of 5-10% was observed in most LP16 and LP20 ADC treatment groups.

### Plasma Cytokine Analysis

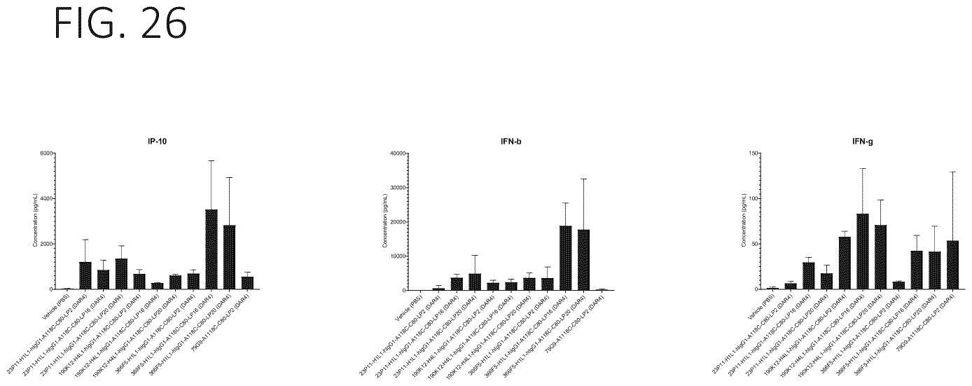

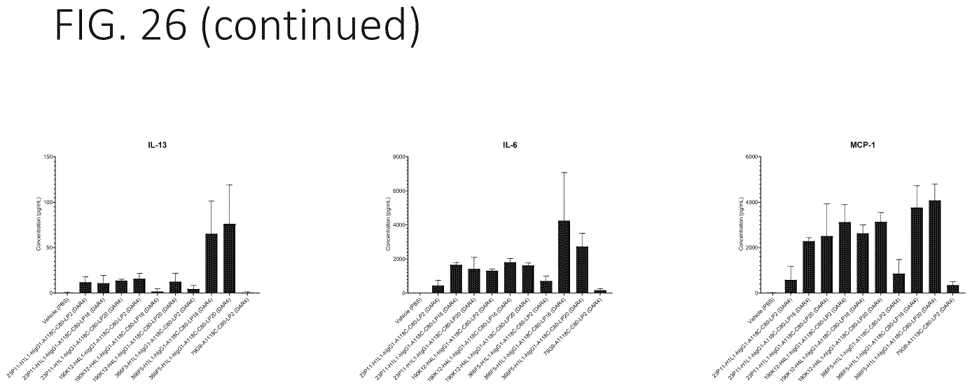

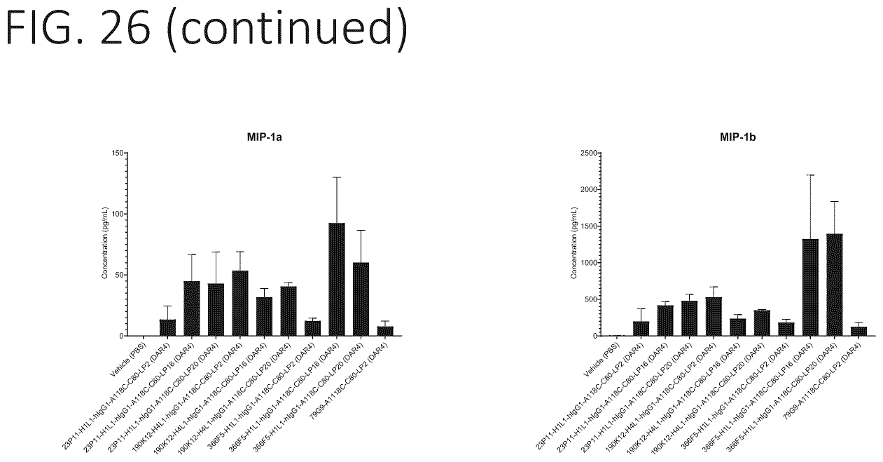

Mouse plasma was collected 5 hours post treatment. Cytokine concentrations were measured and quantified using a Luminex xMAP multiplex kit (FIG. 26). These data show that LP16 and LP20 ADC-treated mice produced cytokines [INF-β, INF-γ, IL-6] at higher levels than LP2 ADC-treated mice, regardless of which anti-CD73 clone was incorporated in the ADC, in this Ca-OV-3-T2 model.

### Anti-Tumor Activity of Anti-CD73-LP ADCs in BC HCC1954 Xenograft Mouse Model (STING-012 Study)

### Tumor Growth and Body Weight Change

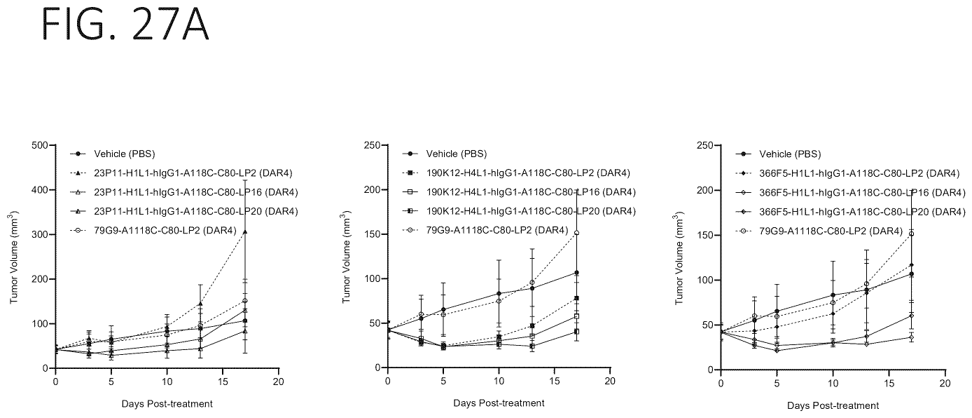

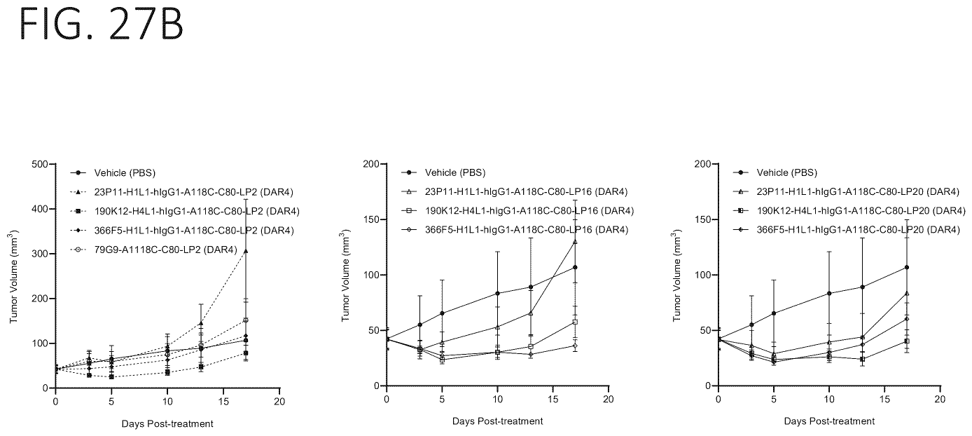

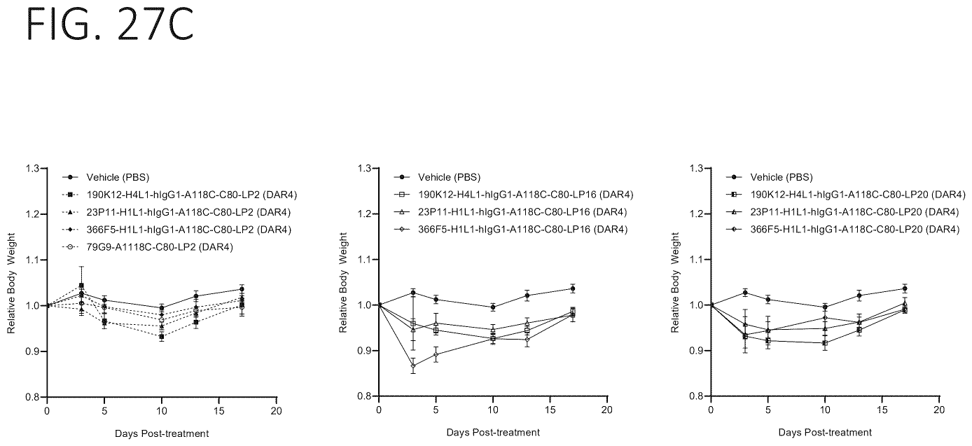

Anti-CD73-LP ADCs were administered as a single bolus IV dose to FoxnI nude mice bearing orthotopic BC HCC1954 tumors. Average tumor volumes of the treatment groups were compared with control group (vehicle/PBS) and body weight changes were recorded (FIG. 27). This study showed that treatment with LP16 and LP20 ADCs leads to better anti-tumor activity than LP2 ADCs. This observation was most pronounced with the 23P11-H1L1 and 366F5-H1L1 clones. 190K12-H4L1 ADCs (LP2, LP16, LP20) showed strong anti-tumor responses in the first 10-12 days following treatment, as did the 366F5-H1L1 (LP16, LP20) ADCs. A slight acute body weight dip (5-10%) was observed in mice treated with LP16 and LP20 ADCs.

### Plasma Cytokine Analysis of STING-012-2023

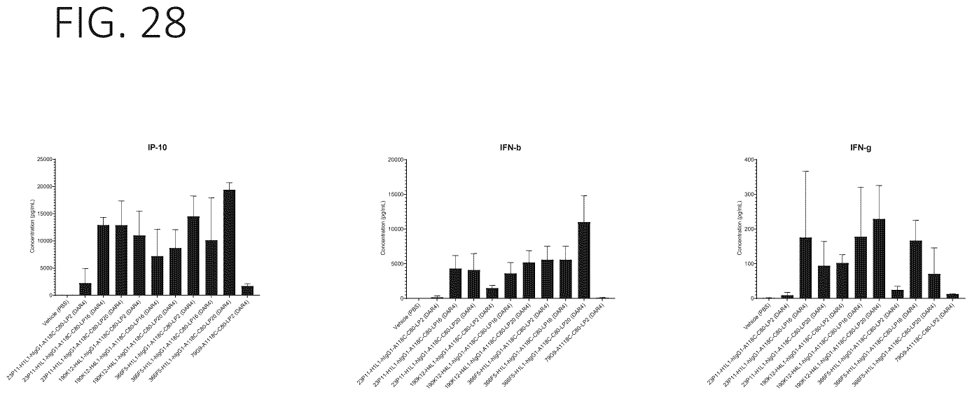

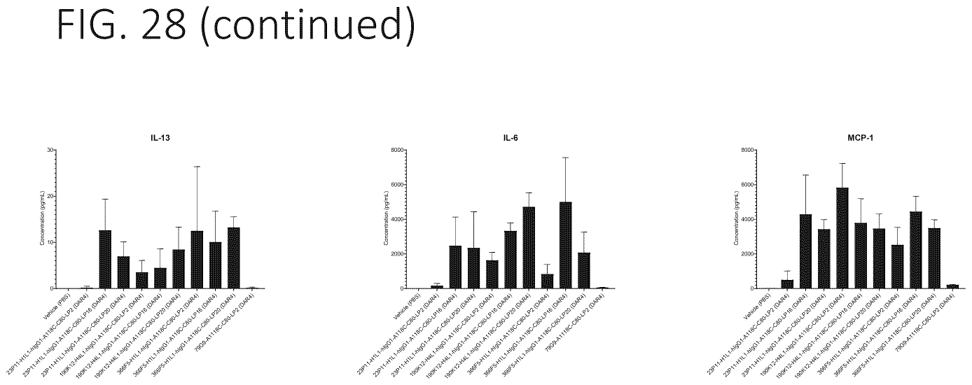

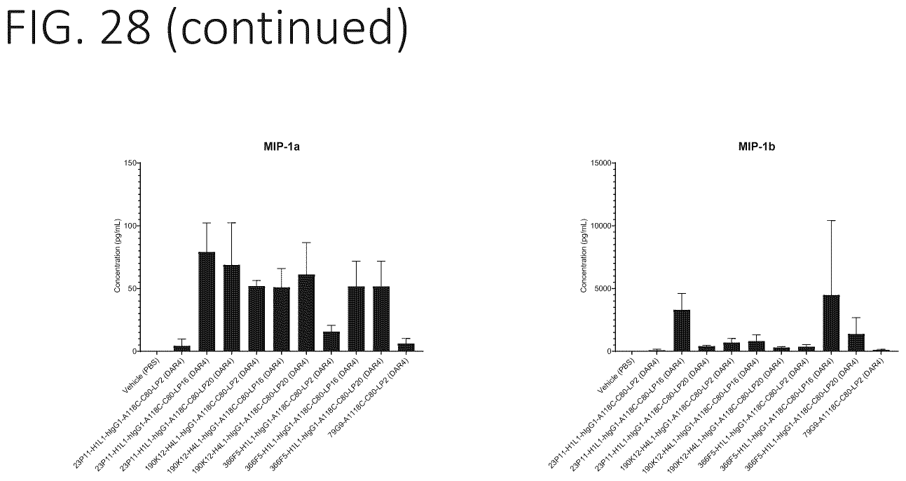

Mouse plasma was collected 5 hours post treatment. Cytokine concentrations were measured and quantified using a Luminex xMAP multiplex kit (FIG. 28). The analysis demonstrated the presence of STING pathway-specific cytokines five hours post treatment. These data show that LP16 and LP20 ADC-treated mice produced cytokines [INF-β, INF-γ, IL-6] at higher levels than LP2 ADC-treated mice, regardless of which anti-CD73 clone was incorporated in the ADC, in this HCC1954 model.

### Example 6. Further Humanization of 23P11

Clone 23PI11 contains rabbit residues in several framework and CDR regions in the VH and VL that may be changed to human residues to reduce potential immunogenicity without impacting antigen binding or thermal stability. Amino acid and nucleic acid sequences of the further humanized antibodies are set forth in Tables 24 and 25.

TABLE 24
Prophetic amino acid sequences of 23P11 mAb variable regions

| IgG chain | Variant | SEQ ID NO | Amino acid sequence |
|---|---|---|---|
| HC | H2 | 85 | EVQLVESGGGLIQPGGSLRLSCAASGFTLSNYYINW |
| | | | VRQAPGKGLEWIGFIPMYGTTHYANWAKGRFTISRD |
| | | | NSKNTLYLQMNSLRAEDTAVYYCARGIASMFYPSIW |
| | | | GQGTLVTVSS |
| HC | H3 | 281 | EVQLVESGGGLIQPGGSLRLSCAASGFSVSNYYINW |
| | | | VRQAPGKGLEWIGFIPMYGTTHYANWAKGRFTISRD |
| | | | NSKNTLYLQMNSLRAEDTAVYYCARGIASMFYPSIW |
| | | | GQGTLVTVSS |
| HC | H4 | 282 | EVQLVESGGGLIQPGGSLRLSCAASGFTVSNYYINW |
| | | | VRQAPGKGLEWIGFIPMYGTTHYANWAKGRFTISRD |
| | | | NSKNTLYLQMNSLRAEDTAVYYCARGIASMFYPSIW |
| | | | GQGTLVTVSS |
| HC | H5 | 283 | EVQLVESGGGLIQPGGSLRLSCAASGFSLSNYYINW |
| | | | VRQAPGKGLEWVGFIPMYGTTHYANWAKGRFTISRD |
| | | | NSKNTLYLQMNSLRAEDTAVYYCARGIASMFYPSIW |
| | | | GQGTLVTVSS |
| HC | H6 | 284 | EVQLVESGGGLIQPGGSLRLSCAASGFSLSNYYINW |
| | | | VRQAPGKGLEWIGFIPMYGTTHYADSVKGRFTISRD |
| | | | NSKNTLYLQMNSLRAEDTAVYYCARGIASMFYPSIW |
| | | | GQGTLVTVSS |
| HC | H7 | 285 | EVQLVESGGGLIQPGGSLRLSCAASGFSLSNYYINW |
| | | | VRQAPGKGLEWIGFIPMYGTTHYANSVKGRFTISRD |
| | | | NSKNTLYLQMNSLRAEDTAVYYCARGIASMFYPSIW |
| | | | GQGTLVTVSS |
| HC | H8 | 286 | EVQLVESGGGLIQPGGSLRLSCAASGFSLSNYYINW |
| | | | VRQAPGKGLEWIGFIPMYGTTHYANWVKGRFTISRD |
| | | | NSKNTLYLQMNSLRAEDTAVYYCARGIASMFYPSIW |
| | | | GQGTLVTVSS |
| HC | H9 | 287 | EVQLVESGGGLIQPGGSLRLSCAASGFTLSNYYINW |
| | | | VRQAPGKGLEWIGFIPMYGTTHYADSVKGRFTISRD |
| | | | NSKNTLYLQMNSLRAEDTAVYYCARGIASMFYPSIW |
| | | | GQGTLVTVSS |
| HC | H10 | 93 | EVQLVESGGGLIQPGGSLRLSCAASGFTLSNYYINW |
| | | | VRQAPGKGLEWIGFIPMYGTTHYANSVKGRFTISRD |
| | | | NSKNTLYLQMNSLRAEDTAVYYCARGIASMFYPSIW |
| | | | GQGTLVTVSS |
| HC | H11 | 94 | EVQLVESGGGLIQPGGSLRLSCAASGFTLSNYYINW |
| | | | VRQAPGKGLEWIGFIPMYGTTHYANWVKGRFTISRD |
| | | | NSKNTLYLQMNSLRAEDTAVYYCARGIASMFYPSIW |
| | | | GQGTLVTVSS |
| HC | H12 | 95 | EVQLVESGGGLIQPGGSLRLSCAASGFTVSNYYINW |
| | | | VRQAPGKGLEWIGFIPMYGTTHYADSVKGRFTISRD |
| | | | NSKNTLYLQMNSLRAEDTAVYYCARGIASMFYPSIW |
| | | | GQGTLVTVSS |
| HC | H13 | 96 | EVQLVESGGGLIQPGGSLRLSCAASGFTVSNYYINW |
| | | | VRQAPGKGLEWIGFIPMYGTTHYANSVKGRFTISRD |
| | | | NSKNTLYLQMNSLRAEDTAVYYCARGIASMFYPSIW |
| | | | GQGTLVTVSS |
| HC | H14 | 97 | EVQLVESGGGLIQPGGSLRLSCAASGFTVSNYYINW |
| | | | VRQAPGKGLEWIGFIPMYGTTHYANWVKGRFTISRD |
| | | | NSKNTLYLQMNSLRAEDTAVYYCARGIASMFYPSIW |
| | | | GQGTLVTVSS |
| HC | H15 | 98 | EVQLVESGGGLIQPGGSLRLSCAASGFTLSNYYINW |
| | | | VRQAPGKGLEWVGFIPMYGTTHYADSVKGRFTISRD |
| | | | NSKNTLYLQMNSLRAEDTAVYYCARGIASMFYPSIW |
| | | | GQGTLVTVSS |
| HC | H16 | 99 | EVQLVESGGGLIQPGGSLRLSCAASGFTLSNYYINW |
| | | | VRQAPGKGLEWVGFIPMYGTTHYANSVKGRFTISRD |
| | | | NSKNTLYLQMNSLRAEDTAVYYCARGIASMFYPSIW |
| | | | GQGTLVTVSS |
| HC | H17 | 100 | EVQLVESGGGLIQPGGSLRLSCAASGFTLSNYYINW |
| | | | VRQAPGKGLEWVGFIPMYGTTHYANWVKGRFTISRD |
| | | | NSKNTLYLQMNSLRAEDTAVYYCARGIASMFYPSIW |
| | | | GQGTLVTVSS |
| HC | H18 | 101 | EVQLVESGGGLIQPGGSLRLSCAASGFTVSNYYINW |
| | | | VRQAPGKGLEWVGFIPMYGTTHYADSVKGRFTISRD |
| | | | NSKNTLYLQMNSLRAEDTAVYYCARGIASMFYPSIW |
| | | | GQGTLVTVSS |
| HC | H19 | 102 | EVQLVESGGGLIQPGGSLRLSCAASGFTVSNYYINW |
| | | | VRQAPGKGLEWVGFIPMYGTTHYANSVKGRFTISRD |
| | | | NSKNTLYLQMNSLRAEDTAVYYCARGIASMFYPSIW |
| | | | GQGTLVTVSS |
| HC | H20 | 103 | EVQLVESGGGLIQPGGSLRLSCAASGFTVSNYYINW |
| | | | VRQAPGKGLEWVGFIPMYGTTHYANWVKGRFTISRD |
| | | | NSKNTLYLQMNSLRAEDTAVYYCARGIASMFYPSIW |
| | | | GQGTLVTVSS |
| LC | L2 | 104 | DVVMTQSPSSLSASVGDRVTITCQASEDIHGYLNWY |
| | | | QQKPGKAPKLLIYYASTLASGVPSRFSGSGSGTDFT |
| | | | LTISSLQPEDFATYYCLGDGSTSGITFGQGTKVEIK |
| LC | L3 | 105 | DVVMTQSPSSLSASVGDRVTITCQASEDIHGYLAWY |
| | | | QQKPGKAPKLLIYYASTLQSGVPSRFSGSGSGTDFT |
| | | | LTISSLQPEDFATYYCLGDGSTSGITFGQGTKVEIK |
| LC | L4 | 106 | DVVMTQSPSSLSASVGDRVTITCQASEDIHGYLNWY |
| | | | QQKPGKAPKLLIYYASTLQSGVPSRFSGSGSGTDET |
| | | | LTISSLQPEDFATYYCLGDGSTSGITFGQGTKVEIK |
| LC | L5 | 107 | DVVMTQSPSSLSASVGDRVTITCRASEDIHGYLAWY |
| | | | QQKPGKAPKLLIYYASTLASGVPSRFSGSGSGTDFT |
| | | | LTISSLQPEDFATYYCLGDGSTSGITFGQGTKVEIK |
| LC | L6 | 108 | DVVMTQSPSSLSASVGDRVTITCRASEDIHGYLNWY |
| | | | QQKPGKAPKLLIYYASTLASGVPSRFSGSGSGTDFT |
| | | | LTISSLQPEDFATYYCLGDGSTSGITFGQGTKVEIK |
| LC | L7 | 109 | DVVMTQSPSSLSASVGDRVTITCRASEDIHGYLAWY |
| | | | QQKPGKAPKLLIYYASTLQSGVPSRFSGSGSGTDFT |
| | | | LTISSLQPEDFATYYCLGDGSTSGITFGQGTKVEIK |
| LC | L8 | 110 | DVVMTQSPSSLSASVGDRVTITCRASEDIHGYLNWY |
| | | | QQKPGKAPKLLIYYASTLQSGVPSRFSGSGSGTDET |
| | | | LTISSLQPEDFATYYCLGDGSTSGITFGQGTKVEIK |

Bolded text indicates amino acid positions corresponding to CDR sequences according to the Kabat system; underlined text indicates amino acid positions corresponding to CDR sequences according to the IMGT system.

TABLE 25
Nucleic acid sequences encoding the prophetic 23P11 mAb variable regions

| IgG chain | Variant | SEQ ID NO | Nucleic acid sequence |
|---|---|---|---|
| HC | H2 | 214 | GAGGTGCAGCTGGTGGAGAGCGGCGGCGGCCTGATC |
| | | | CAGCCCGGCGGCAGCCTGAGGCTGAGCTGCGCCGCC |
| | | | AGCGGCTTCACCCTGAGCAACTACTACATCAACTGG |
| | | | GTGAGGCAGGCCCCCGGCAAGGGCCTGGAGTGGATC |
| | | | GGCTTCATCCCCATGTACGGCACCACCCACTACGCC |
| | | | AACTGGGCCAAGGGCAGGTTCACCATCAGCAGGGAC |
| | | | AACAGCAAGAACACCCTGTACCTGCAGATGAACAGC |
| | | | CTGAGGGCCGAGGACACCGCCGTGTACTACTGCGCC |
| | | | AGGGGCATCGCCAGCATGTTCTACCCCAGCATCTGG |
| | | | GGCCAGGGCACCCTGGTGACCGTGAGCAGC |
| HC | H3 | 215 | GAGGTGCAGCTGGTGGAGAGCGGCGGCGGCCTGATC |
| | | | CAGCCCGGCGGCAGCCTGAGGCTGAGCTGCGCCGCC |
| | | | AGCGGCTTCAGCGTGAGCAACTACTACATCAACTGG |
| | | | GTGAGGCAGGCCCCCGGCAAGGGCCTGGAGTGGATC |
| | | | GGCTTCATCCCCATGTACGGCACCACCCACTACGCC |
| | | | AACTGGGCCAAGGGCAGGTTCACCATCAGCAGGGAC |
| | | | AACAGCAAGAACACCCTGTACCTGCAGATGAACAGC |
| | | | CTGAGGGCCGAGGACACCGCCGTGTACTACTGCGCC |
| | | | AGGGGCATCGCCAGCATGTTCTACCCCAGCATCTGG |
| | | | GGCCAGGGCACCCTGGTGACCGTGAGCAGC |
| HC | H4 | 216 | GAGGTGCAGCTGGTGGAGAGCGGCGGCGGCCTGATC |
| | | | CAGCCCGGCGGCAGCCTGAGGCTGAGCTGCGCCGCC |
| | | | AGCGGCTTCACCGTGAGCAACTACTACATCAACTGG |
| | | | GTGAGGCAGGCCCCCGGCAAGGGCCTGGAGTGGATC |
| | | | GGCTTCATCCCCATGTACGGCACCACCCACTACGCC |
| | | | AACTGGGCCAAGGGCAGGTTCACCATCAGCAGGGAC |
| | | | AACAGCAAGAACACCCTGTACCTGCAGATGAACAGC |
| | | | CTGAGGGCCGAGGACACCGCCGTGTACTACTGCGCC |
| | | | AGGGGCATCGCCAGCATGTTCTACCCCAGCATCTGG |
| | | | GGCCAGGGCACCCTGGTGACCGTGAGCAGC |
| HC | H5 | 217 | GAGGTGCAGCTGGTGGAGAGCGGCGGCGGCCTGATC |
| | | | CAGCCCGGCGGCAGCCTGAGGCTGAGCTGCGCCGCC |
| | | | AGCGGCTTCAGCCTGAGCAACTACTACATCAACTGG |
| | | | GTGAGGCAGGCCCCCGGCAAGGGCCTGGAGTGGGTG |
| | | | GGCTTCATCCCCATGTACGGCACCACCCACTACGCC |
| | | | AACTGGGCCAAGGGCAGGTTCACCATCAGCAGGGAC |
| | | | AACAGCAAGAACACCCTGTACCTGCAGATGAACAGC |
| | | | CTGAGGGCCGAGGACACCGCCGTGTACTACTGCGCC |
| | | | AGGGGCATCGCCAGCATGTTCTACCCCAGCATCTGG |
| | | | GGCCAGGGCACCCTGGTGACCGTGAGCAGC |
| HC | H6 | 218 | GAGGTGCAGCTGGTGGAGAGCGGCGGCGGCCTGATC |
| | | | CAGCCCGGCGGCAGCCTGAGGCTGAGCTGCGCCGCC |
| | | | AGCGGCTTCAGCCTGAGCAACTACTACATCAACTGG |
| | | | GTGAGGCAGGCCCCCGGCAAGGGCCTGGAGTGGATC |
| | | | GGCTTCATCCCCATGTACGGCACCACCCACTACGCC |
| | | | GACAGCGTGAAGGGCAGGTTCACCATCAGCAGGGAC |
| | | | AACAGCAAGAACACCCTGTACCTGCAGATGAACAGC |
| | | | CTGAGGGCCGAGGACACCGCCGTGTACTACTGCGCC |
| | | | AGGGGCATCGCCAGCATGTTCTACCCCAGCATCTGG |
| | | | GGCCAGGGCACCCTGGTGACCGTGAGCAGC |
| HC | H7 | 219 | GAGGTGCAGCTGGTGGAGAGCGGCGGCGGCCTGATC |
| | | | CAGCCCGGCGGCAGCCTGAGGCTGAGCTGCGCCGCC |
| | | | AGCGGCTTCAGCCTGAGCAACTACTACATCAACTGG |
| | | | GTGAGGCAGGCCCCCGGCAAGGGCCTGGAGTGGATC |
| | | | GGCTTCATCCCCATGTACGGCACCACCCACTACGCC |
| | | | AACAGCGTGAAGGGCAGGTTCACCATCAGCAGGGAC |
| | | | AACAGCAAGAACACCCTGTACCTGCAGATGAACAGC |
| | | | CTGAGGGCCGAGGACACCGCCGTGTACTACTGCGCC |
| | | | AGGGGCATCGCCAGCATGTTCTACCCCAGCATCTGG |
| | | | GGCCAGGGCACCCTGGTGACCGTGAGCAGC |
| HC | H8 | 220 | GAGGTGCAGCTGGTGGAGAGCGGCGGCGGCCTGATC |
| | | | CAGCCCGGCGGCAGCCTGAGGCTGAGCTGCGCCGCC |
| | | | AGCGGCTTCAGCCTGAGCAACTACTACATCAACTGG |
| | | | GTGAGGCAGGCCCCCGGCAAGGGCCTGGAGTGGATC |
| | | | GGCTTCATCCCCATGTACGGCACCACCCACTACGCC |
| | | | AACTGGGTGAAGGGCAGGTTCACCATCAGCAGGGAC |
| | | | AACAGCAAGAACACCCTGTACCTGCAGATGAACAGC |
| | | | CTGAGGGCCGAGGACACCGCCGTGTACTACTGCGCC |
| | | | AGGGGCATCGCCAGCATGTTCTACCCCAGCATCTGG |
| | | | GGCCAGGGCACCCTGGTGACCGTGAGCAGC |
| HC | H9 | 221 | GAGGTGCAGCTGGTGGAGAGCGGCGGCGGCCTGATC |
| | | | CAGCCCGGCGGCAGCCTGAGGCTGAGCTGCGCCGCC |
| | | | AGCGGCTTCACCCTGAGCAACTACTACATCAACTGG |
| | | | GTGAGGCAGGCCCCCGGCAAGGGCCTGGAGTGGATC |
| | | | GGCTTCATCCCCATGTACGGCACCACCCACTACGCC |
| | | | GACAGCGTGAAGGGCAGGTTCACCATCAGCAGGGAC |
| | | | AACAGCAAGAACACCCTGTACCTGCAGATGAACAGC |
| | | | CTGAGGGCCGAGGACACCGCCGTGTACTACTGCGCC |
| | | | AGGGGCATCGCCAGCATGTTCTACCCCAGCATCTGG |
| | | | GGCCAGGGCACCCTGGTGACCGTGAGCAGC |
| HC | H10 | 222 | GAGGTGCAGCTGGTGGAGAGCGGCGGCGGCCTGATC |
| | | | CAGCCCGGCGGCAGCCTGAGGCTGAGCTGCGCCGCC |
| | | | AGCGGCTTCACCCTGAGCAACTACTACATCAACTGG |
| | | | GTGAGGCAGGCCCCCGGCAAGGGCCTGGAGTGGATC |
| | | | GGCTTCATCCCCATGTACGGCACCACCCACTACGCC |
| | | | AACAGCGTGAAGGGCAGGTTCACCATCAGCAGGGAC |
| | | | AACAGCAAGAACACCCTGTACCTGCAGATGAACAGC |
| | | | CTGAGGGCCGAGGACACCGCCGTGTACTACTGCGCC |
| | | | AGGGGCATCGCCAGCATGTTCTACCCCAGCATCTGG |
| | | | GGCCAGGGCACCCTGGTGACCGTGAGCAGC |
| HC | H11 | 223 | GAGGTGCAGCTGGTGGAGAGCGGCGGCGGCCTGATC |
| | | | CAGCCCGGCGGCAGCCTGAGGCTGAGCTGCGCCGCC |
| | | | AGCGGCTTCACCCTGAGCAACTACTACATCAACTGG |
| | | | GTGAGGCAGGCCCCCGGCAAGGGCCTGGAGTGGATC |
| | | | GGCTTCATCCCCATGTACGGCACCACCCACTACGCC |
| | | | AACTGGGTGAAGGGCAGGTTCACCATCAGCAGGGAC |
| | | | AACAGCAAGAACACCCTGTACCTGCAGATGAACAGC |
| | | | CTGAGGGCCGAGGACACCGCCGTGTACTACTGCGCC |
| | | | AGGGGCATCGCCAGCATGTTCTACCCCAGCATCTGG |
| | | | GGCCAGGGCACCCTGGTGACCGTGAGCAGC |
| HC | H12 | 224 | GAGGTGCAGCTGGTGGAGAGCGGCGGCGGCCTGATC |
| | | | CAGCCCGGCGGCAGCCTGAGGCTGAGCTGCGCCGCC |
| | | | AGCGGCTTCACCGTGAGCAACTACTACATCAACTGG |
| | | | GTGAGGCAGGCCCCCGGCAAGGGCCTGGAGTGGATC |
| | | | GGCTTCATCCCCATGTACGGCACCACCCACTACGCC |
| | | | GACAGCGTGAAGGGCAGGTTCACCATCAGCAGGGAC |
| | | | AACAGCAAGAACACCCTGTACCTGCAGATGAACAGC |
| | | | CTGAGGGCCGAGGACACCGCCGTGTACTACTGCGCC |
| | | | AGGGGCATCGCCAGCATGTTCTACCCCAGCATCTGG |
| | | | GGCCAGGGCACCCTGGTGACCGTGAGCAGC |
| HC | H13 | 225 | GAGGTGCAGCTGGTGGAGAGCGGCGGCGGCCTGATC |
| | | | CAGCCCGGCGGCAGCCTGAGGCTGAGCTGCGCCGCC |
| | | | AGCGGCTTCACCGTGAGCAACTACTACATCAACTGG |
| | | | GTGAGGCAGGCCCCCGGCAAGGGCCTGGAGTGGATC |
| | | | GGCTTCATCCCCATGTACGGCACCACCCACTACGCC |
| | | | AACAGCGTGAAGGGCAGGTTCACCATCAGCAGGGAC |
| | | | AACAGCAAGAACACCCTGTACCTGCAGATGAACAGC |
| | | | CTGAGGGCCGAGGACACCGCCGTGTACTACTGCGCC |
| | | | AGGGGCATCGCCAGCATGTTCTACCCCAGCATCTGG |
| | | | GGCCAGGGCACCCTGGTGACCGTGAGCAGC |
| HC | H14 | 226 | GAGGTGCAGCTGGTGGAGAGCGGCGGCGGCCTGATC |
| | | | CAGCCCGGCGGCAGCCTGAGGCTGAGCTGCGCCGCC |
| | | | AGCGGCTTCACCGTGAGCAACTACTACATCAACTGG |
| | | | GTGAGGCAGGCCCCCGGCAAGGGCCTGGAGTGGATC |
| | | | GGCTTCATCCCCATGTACGGCACCACCCACTACGCC |
| | | | AACTGGGTGAAGGGCAGGTTCACCATCAGCAGGGAC |
| | | | AACAGCAAGAACACCCTGTACCTGCAGATGAACAGC |
| | | | CTGAGGGCCGAGGACACCGCCGTGTACTACTGCGCC |
| | | | AGGGGCATCGCCAGCATGTTCTACCCCAGCATCTGG |
| | | | GGCCAGGGCACCCTGGTGACCGTGAGCAGC |
| HC | H15 | 227 | GAGGTGCAGCTGGTGGAGAGCGGCGGCGGCCTGATC |
| | | | CAGCCCGGCGGCAGCCTGAGGCTGAGCTGCGCCGCC |
| | | | AGCGGCTTCACCCTGAGCAACTACTACATCAACTGG |
| | | | GTGAGGCAGGCCCCCGGCAAGGGCCTGGAGTGGGTG |
| | | | GGCTTCATCCCCATGTACGGCACCACCCACTACGCC |
| | | | GACAGCGTGAAGGGCAGGTTCACCATCAGCAGGGAC |
| | | | AACAGCAAGAACACCCTGTACCTGCAGATGAACAGC |
| | | | CTGAGGGCCGAGGACACCGCCGTGTACTACTGCGCC |
| | | | AGGGGCATCGCCAGCATGTTCTACCCCAGCATCTGG |
| | | | GGCCAGGGCACCCTGGTGACCGTGAGCAGC |
| HC | H16 | 228 | GAGGTGCAGCTGGTGGAGAGCGGCGGCGGCCTGATC |
| | | | CAGCCCGGCGGCAGCCTGAGGCTGAGCTGCGCCGCC |
| | | | AGCGGCTTCACCCTGAGCAACTACTACATCAACTGG |
| | | | GTGAGGCAGGCCCCCGGCAAGGGCCTGGAGTGGGTG |
| | | | GGCTTCATCCCCATGTACGGCACCACCCACTACGCC |
| | | | AACAGCGTGAAGGGCAGGTTCACCATCAGCAGGGAC |
| | | | AACAGCAAGAACACCCTGTACCTGCAGATGAACAGC |
| | | | CTGAGGGCCGAGGACACCGCCGTGTACTACTGCGCC |
| | | | AGGGGCATCGCCAGCATGTTCTACCCCAGCATCTGG |
| | | | GGCCAGGGCACCCTGGTGACCGTGAGCAGC |
| HC | H17 | 229 | GAGGTGCAGCTGGTGGAGAGCGGCGGCGGCCTGATC |
| | | | CAGCCCGGCGGCAGCCTGAGGCTGAGCTGCGCCGCC |
| | | | AGCGGCTTCACCCTGAGCAACTACTACATCAACTGG |
| | | | GTGAGGCAGGCCCCCGGCAAGGGCCTGGAGTGGGTG |
| | | | GGCTTCATCCCCATGTACGGCACCACCCACTACGCC |
| | | | AACTGGGTGAAGGGCAGGTTCACCATCAGCAGGGAC |
| | | | AACAGCAAGAACACCCTGTACCTGCAGATGAACAGC |
| | | | CTGAGGGCCGAGGACACCGCCGTGTACTACTGCGCC |
| | | | AGGGGCATCGCCAGCATGTTCTACCCCAGCATCTGG |
| | | | GGCCAGGGCACCCTGGTGACCGTGAGCAGC |
| HC | H18 | 230 | GAGGTGCAGCTGGTGGAGAGCGGCGGCGGCCTGATC |
| | | | CAGCCCGGCGGCAGCCTGAGGCTGAGCTGCGCCGCC |
| | | | AGCGGCTTCACCGTGAGCAACTACTACATCAACTGG |
| | | | GTGAGGCAGGCCCCCGGCAAGGGCCTGGAGTGGGTG |
| | | | GGCTTCATCCCCATGTACGGCACCACCCACTACGCC |
| | | | GACAGCGTGAAGGGCAGGTTCACCATCAGCAGGGAC |
| | | | AACAGCAAGAACACCCTGTACCTGCAGATGAACAGC |
| | | | CTGAGGGCCGAGGACACCGCCGTGTACTACTGCGCC |
| | | | AGGGGCATCGCCAGCATGTTCTACCCCAGCATCTGG |
| | | | GGCCAGGGCACCCTGGTGACCGTGAGCAGC |
| HC | H19 | 231 | GAGGTGCAGCTGGTGGAGAGCGGCGGCGGCCTGATC |
| | | | CAGCCCGGCGGCAGCCTGAGGCTGAGCTGCGCCGCC |
| | | | AGCGGCTTCACCGTGAGCAACTACTACATCAACTGG |
| | | | GTGAGGCAGGCCCCCGGCAAGGGCCTGGAGTGGGTG |
| | | | GGCTTCATCCCCATGTACGGCACCACCCACTACGCC |
| | | | AACAGCGTGAAGGGCAGGTTCACCATCAGCAGGGAC |
| | | | AACAGCAAGAACACCCTGTACCTGCAGATGAACAGC |
| | | | CTGAGGGCCGAGGACACCGCCGTGTACTACTGCGCC |
| | | | AGGGGCATCGCCAGCATGTTCTACCCCAGCATCTGG |
| | | | GGCCAGGGCACCCTGGTGACCGTGAGCAGC |
| HC | H20 | 232 | GAGGTGCAGCTGGTGGAGAGCGGCGGCGGCCTGATC |
| | | | CAGCCCGGCGGCAGCCTGAGGCTGAGCTGCGCCGCC |
| | | | AGCGGCTTCACCGTGAGCAACTACTACATCAACTGG |
| | | | GTGAGGCAGGCCCCCGGCAAGGGCCTGGAGTGGGTG |
| | | | GGCTTCATCCCCATGTACGGCACCACCCACTACGCC |
| | | | AACTGGGTGAAGGGCAGGTTCACCATCAGCAGGGAC |
| | | | AACAGCAAGAACACCCTGTACCTGCAGATGAACAGC |
| | | | CTGAGGGCCGAGGACACCGCCGTGTACTACTGCGCC |
| | | | AGGGGCATCGCCAGCATGTTCTACCCCAGCATCTGG |
| | | | GGCCAGGGCACCCTGGTGACCGTGAGCAGC |
| LC | L2 | 233 | GACGTGGTGATGACCCAGAGCCCCAGCAGCCTGAGC |
| | | | GCCAGCGTGGGCGACAGGGTGACCATCACCTGCCAG |
| | | | GCCAGCGAGGACATCCACGGCTACCTGAACTGGTAC |
| | | | CAGCAGAAGCCCGGCAAGGCCCCCAAGCTGCTGATC |
| | | | TACTACGCCAGCACCCTGGCCAGCGGCGTGCCCAGC |
| | | | AGGTTCAGCGGCAGCGGCAGCGGCACCGACTTCACC |
| | | | CTGACCATCAGCAGCCTGCAGCCCGAGGACTTCGCC |
| | | | ACCTACTACTGCCTGGGCGACGGCAGCACCAGCGGC |
| | | | ATCACCTTCGGCCAGGGCACCAAGGTGGAGATCAAG |
| LC | L3 | 234 | GACGTGGTGATGACCCAGAGCCCCAGCAGCCTGAGC |
| | | | GCCAGCGTGGGCGACAGGGTGACCATCACCTGCCAG |
| | | | GCCAGCGAGGACATCCACGGCTACCTGGCCTGGTAC |
| | | | CAGCAGAAGCCCGGCAAGGCCCCCAAGCTGCTGATC |
| | | | TACTACGCCAGCACCCTGCAGAGCGGCGTGCCCAGC |
| | | | AGGTTCAGCGGCAGCGGCAGCGGCACCGACTTCACC |
| | | | CTGACCATCAGCAGCCTGCAGCCCGAGGACTTCGCC |
| | | | ACCTACTACTGCCTGGGCGACGGCAGCACCAGCGGC |
| | | | ATCACCTTCGGCCAGGGCACCAAGGTGGAGATCAAG |
| LC | L4 | 235 | GACGTGGTGATGACCCAGAGCCCCAGCAGCCTGAGC |
| | | | GCCAGCGTGGGCGACAGGGTGACCATCACCTGCCAG |
| | | | GCCAGCGAGGACATCCACGGCTACCTGAACTGGTAC |
| | | | CAGCAGAAGCCCGGCAAGGCCCCCAAGCTGCTGATC |
| | | | TACTACGCCAGCACCCTGCAGAGCGGCGTGCCCAGC |
| | | | AGGTTCAGCGGCAGCGGCAGCGGCACCGACTTCACC |
| | | | CTGACCATCAGCAGCCTGCAGCCCGAGGACTTCGCC |
| | | | ACCTACTACTGCCTGGGCGACGGCAGCACCAGCGGC |
| | | | ATCACCTTCGGCCAGGGCACCAAGGTGGAGATCAAG |
| LC | L5 | 236 | GACGTGGTGATGACCCAGAGCCCCAGCAGCCTGAGC |
| | | | GCCAGCGTGGGCGACAGGGTGACCATCACCTGCAGG |
| | | | GCCAGCGAGGACATCCACGGCTACCTGGCCTGGTAC |
| | | | CAGCAGAAGCCCGGCAAGGCCCCCAAGCTGCTGATC |
| | | | TACTACGCCAGCACCCTGGCCAGCGGCGTGCCCAGC |
| | | | AGGTTCAGCGGCAGCGGCAGCGGCACCGACTTCACC |
| | | | CTGACCATCAGCAGCCTGCAGCCCGAGGACTTCGCC |
| | | | ACCTACTACTGCCTGGGCGACGGCAGCACCAGCGGC |
| | | | ATCACCTTCGGCCAGGGCACCAAGGTGGAGATCAAG |
| LC | L6 | 237 | GACGTGGTGATGACCCAGAGCCCCAGCAGCCTGAGC |
| | | | GCCAGCGTGGGCGACAGGGTGACCATCACCTGCAGG |
| | | | GCCAGCGAGGACATCCACGGCTACCTGAACTGGTAC |
| | | | CAGCAGAAGCCCGGCAAGGCCCCCAAGCTGCTGATC |
| | | | TACTACGCCAGCACCCTGGCCAGCGGCGTGCCCAGC |
| | | | AGGTTCAGCGGCAGCGGCAGCGGCACCGACTTCACC |
| | | | CTGACCATCAGCAGCCTGCAGCCCGAGGACTTCGCC |
| | | | ACCTACTACTGCCTGGGCGACGGCAGCACCAGCGGC |
| | | | ATCACCTTCGGCCAGGGCACCAAGGTGGAGATCAAG |
| LC | L7 | 238 | GACGTGGTGATGACCCAGAGCCCCAGCAGCCTGAGC |
| | | | GCCAGCGTGGGCGACAGGGTGACCATCACCTGCAGG |
| | | | GCCAGCGAGGACATCCACGGCTACCTGGCCTGGTAC |
| | | | CAGCAGAAGCCCGGCAAGGCCCCCAAGCTGCTGATC |
| | | | TACTACGCCAGCACCCTGCAGAGCGGCGTGCCCAGC |
| | | | AGGTTCAGCGGCAGCGGCAGCGGCACCGACTTCACC |
| | | | CTGACCATCAGCAGCCTGCAGCCCGAGGACTTCGCC |
| | | | ACCTACTACTGCCTGGGCGACGGCAGCACCAGCGGC |
| | | | ATCACCTTCGGCCAGGGCACCAAGGTGGAGATCAAG |
| LC | L8 | 239 | GACGTGGTGATGACCCAGAGCCCCAGCAGCCTGAGC |
| | | | GCCAGCGTGGGCGACAGGGTGACCATCACCTGCAGG |
| | | | GCCAGCGAGGACATCCACGGCTACCTGAACTGGTAC |
| | | | CAGCAGAAGCCCGGCAAGGCCCCCAAGCTGCTGATC |
| | | | TACTACGCCAGCACCCTGCAGAGCGGCGTGCCCAGC |
| | | | AGGTTCAGCGGCAGCGGCAGCGGCACCGACTTCACC |
| | | | CTGACCATCAGCAGCCTGCAGCCCGAGGACTTCGCC |
| | | | ACCTACTACTGCCTGGGCGACGGCAGCACCAGCGGC |
| | | | ATCACCTTCGGCCAGGGCACCAAGGTGGAGATCAAG |

### Example 7. Anti-CD73 LP54 and LP55 ADC Characterization

### Stability Assays

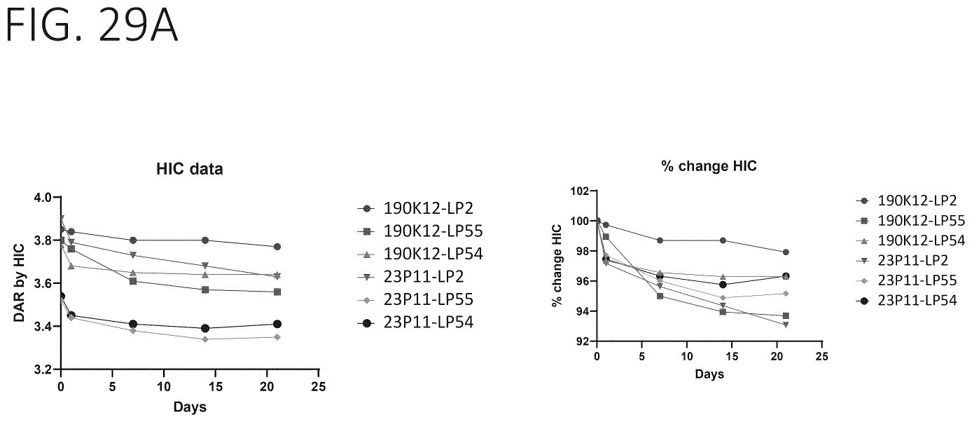

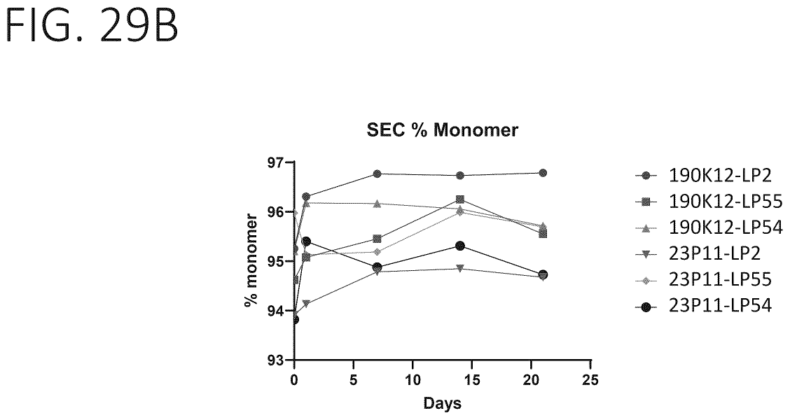

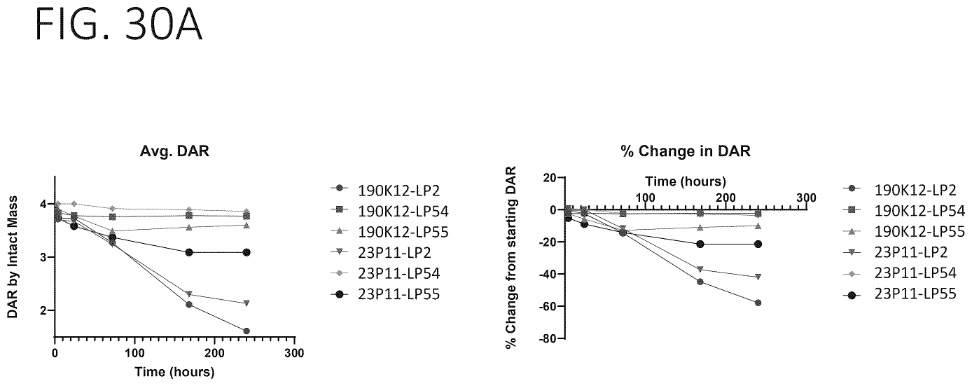

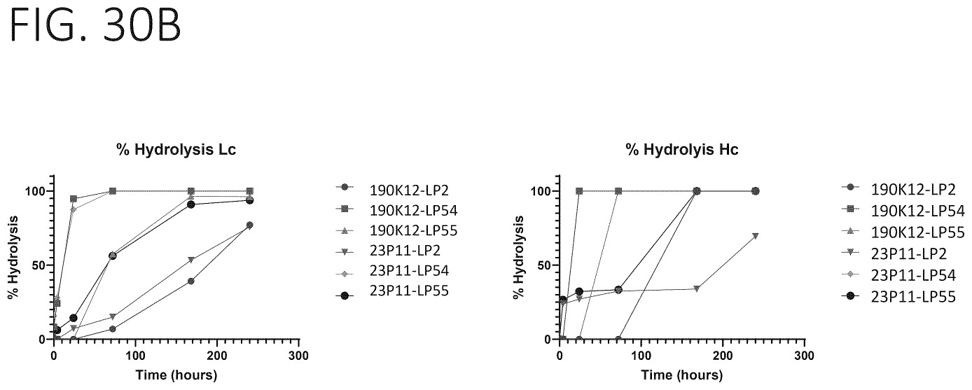

Anti-CD73-LP ADCs were evaluated under thermally stressed conditions in buffer (FIG. 29) and plasma (FIG. 30) to determine whether the spacer and/or the self-immolative units affect the DAR stability. In buffer, very little change in DAR was observed between the three linker-payloads when conjugated to two different antibodies (190K12 and 23PI11), ranging from 2-7% loss in DAR as measured by HIC-HPLC (FIG. 29A). No aggregation was observed over time for any compound as measured by SEC-HPLC (FIG. 29B).

The stability of the ADCs in plasma was also analyzed. A rapid loss in DAR was observed with LP2, reaching 42% or 58% DAR loss by 240 hours. Changing the Unit 1 (MEC) self-immolative moiety to Unit 2 resulted in increased stability, as seen with LP55 (10% and 21.5% DAR loss). The DAR was further stabilized by replacing the maleimidocaproyl (LP2) or Mal-PEG₂ (LP55) spacer units with Mal-Formula (II) (LP54). The DAR for LP54 demonstrated minimal loss of 2.5% or 3.5%.

The increase in plasma stability is likely due to the rapid hydrolysis of the thiosuccinimide in LP54 (FIG. 30B), likely mediated by the Formula (II) moiety. Within 24 hours, the LP54 was 94% hydrolyzed when conjugated to the light chain and 100% when conjugated to the heavy chain. Hydrolysis of LP55 and LP2 was much slower, making these linker-payloads more susceptible to Retro-Michael release of the payload, a major mechanism of DAR instability in ADCs.

### Anti-Tumor Activity of Anti-CD73 LP ADCs in NCI-H2110 Xenograft Mice Models

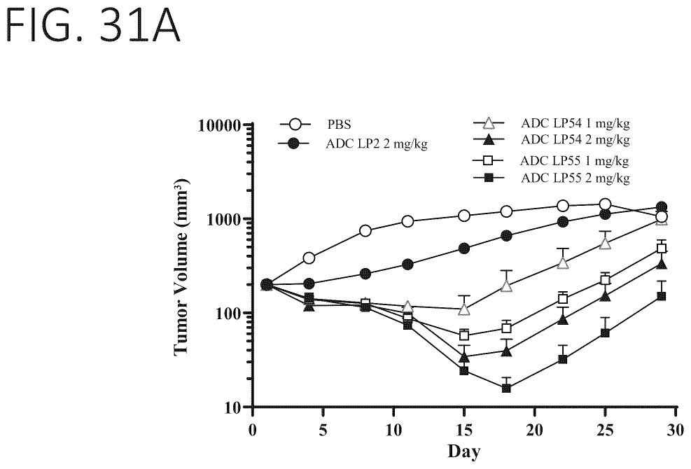

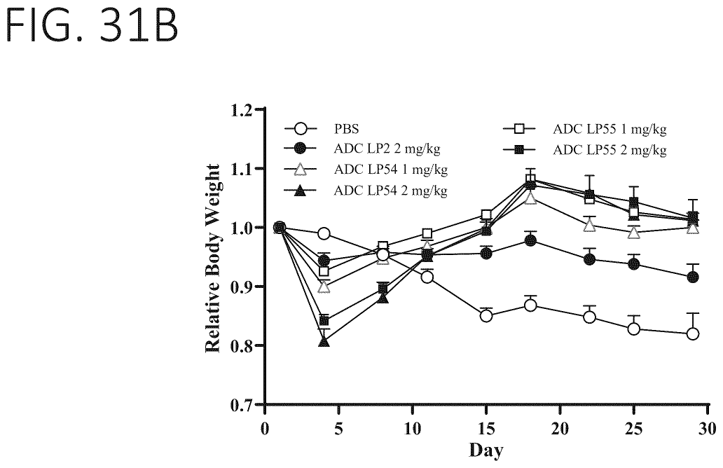

Anti-CD73-LP ADCs were administered as a single IV bolus dose in NCI-H2110 xenograft mice models. 190K12-LP2 (2 mg/kg) induced tumor growth delay with no regression (FIG. 31A). Both 190K12-LP54 (at a dose of 1 mg/kg and 2 mg/kg) and 190K12-LP55 (at a dose of 1 mg/kg and 2 mg/kg) showed tumor regression, and tumor regrowth was observed about 2 weeks after administration. 190K12-LP55 was more potent compared with 190K12-LP54. Relative body weight gradually decreased in the PBS-treated group, while 190K12-LP54 and 190K12-LP55 did not induce marked body weight loss (FIG. 31B).

### Example 8. Synthesis of {4-[(2S)-2-[(2S)-2-[3-(2-{2-[2-(2,5-dioxo-2,5-dihydro-1H-pyrrol-1-yl)acetamido]ethoxy}ethoxy)propanamido]-3-methylbutanamido]propanamido]phenyl}methyl N-({2-[(R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.13,36.128,31.04,8.07,12.019,24.023,27]dotetraconta-5,7,9,11,15,19,21,23,25-nonaene-13-carbonyl]phenyl}methyl)-N-methylcarbamate (LP54)

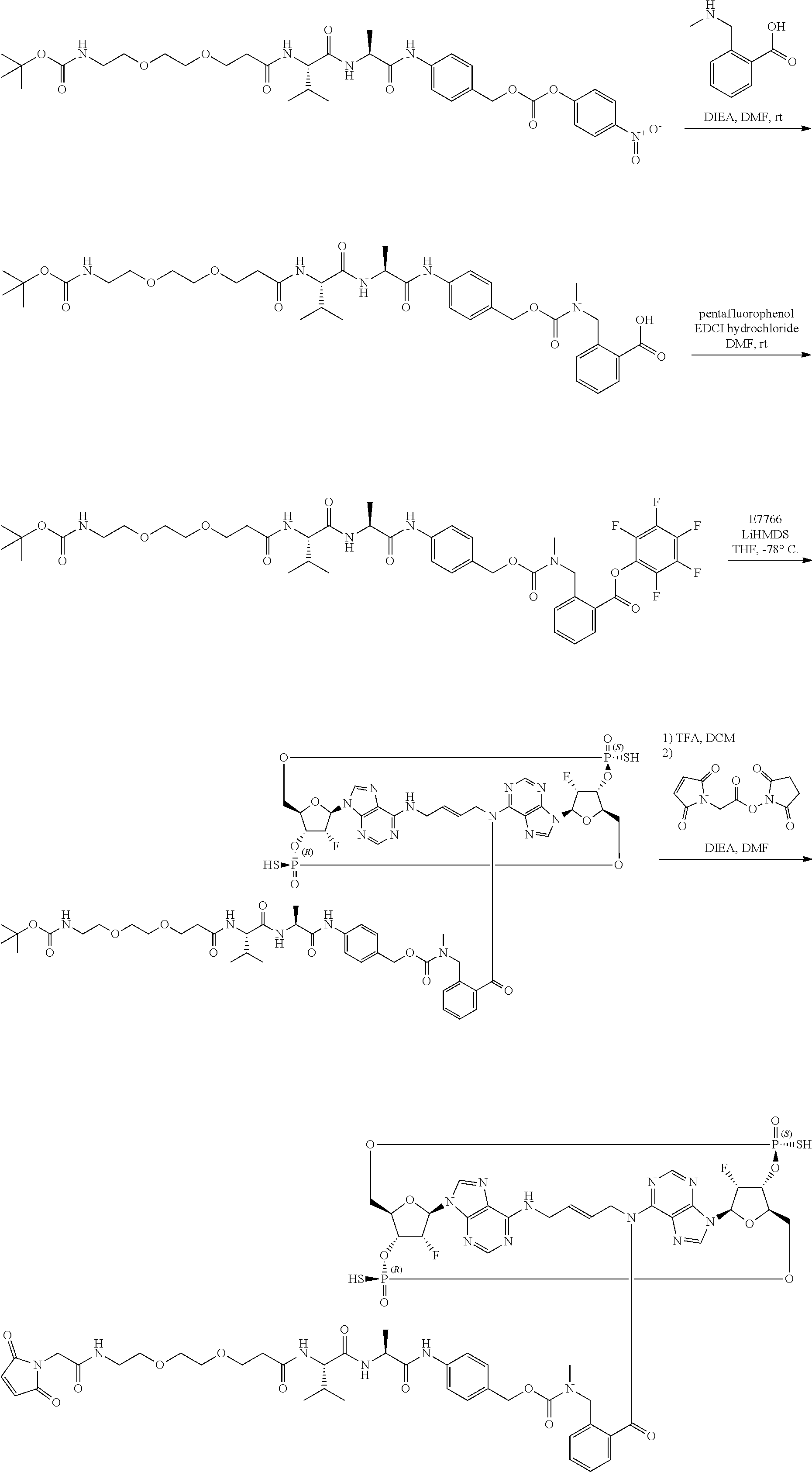

### 8.1 Synthesis of 2-({[({4-[(2S)-2-[(2S)-2-{3-[2-(2-{[(tert-butoxy)carbonyl]amino}ethoxy)ethoxy]propanamido}-3-methylbutanamido]propanamido]phenyl}methoxy)carbonyl](methyl)amino}methyl)benzoic acid

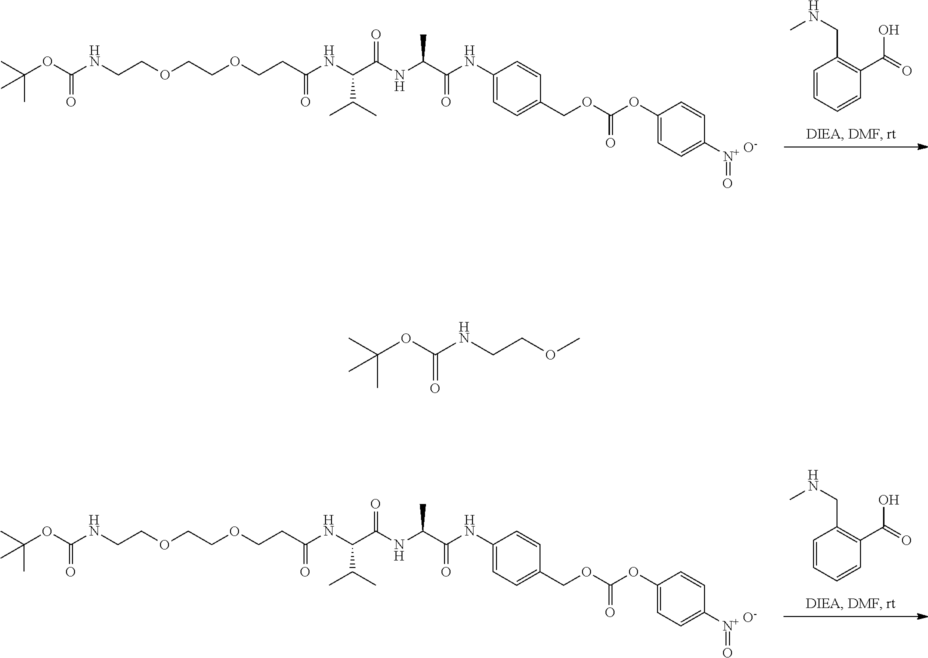

To a solution of {4-[(2S)-2-[(2S)-2-{3-[2-(2-{[(tert-butoxy)carbonyl]amino}ethoxy)ethoxy]propanamido}-3-methylbutanamido]propanamido]phenyl}methyl 4-nitrophenyl carbonate (146 mg, 0.203 mmol) in DMF (2.0 ml) at rt were added 2-[(methylamino)methyl]benzoic acid (37.0 mg, 0.224 mmol) and DIEA (106 μL, 0.61 mmol). The reaction mixture was stirred at room temperature for 125 min, then it was concentrated under reduced pressure. The residue was purified by flash chromatography (0% to 15% MeOH/CHCl₃) to give 2-({[({4-[(2S)-2-[(2S)-2-{3-[2-(2-{[(tert-butoxy)carbonyl]amino}ethoxy)ethoxy]propanamido}-3-methylbutanamido]propanamido]phenyl}methoxy)carbonyl](methyl)amino}methyl)benzoic acid (150 mg) as a colorless oil.

ESI-MS (m/z): 744.65 [M+H]⁺.

¹H NMR (500 MHz, CHLOROFORM-d) δ ppm 0.80-1.09 (m, 6H), 1.34-1.58 (m, 12H), 2.10-2.21 (m, 1H), 2.48-2.65 (m, 2H), 2.76-3.08 (m, 3H), 3.21-3.38 (m, 2H), 3.41-3.64 (m, 6H), 3.65-3.81 (m, 2H), 4.28-4.44 (m, 1H), 4.60-5.36 (m, 5H), 6.24-6.47 (m, 1H), 6.97-7.78 (m, 8H), 7.84-8.19 (m, 1H), 8.70-8.98 (m, 1H), 9.00-9.31 (m, 1H).

### 8.2 Synthesis of 2,3,4,5,6-pentafluorophenyl 2-({[({4-[(2S)-2-[(2S)-2-{3-[2-(2-{[(tert-butoxy)carbonyl]amino}ethoxy)ethoxy]propanamido}-3-methylbutanamido]propanamido]phenyl}methoxy)carbonyl](methyl)amino}methyl)benzoate

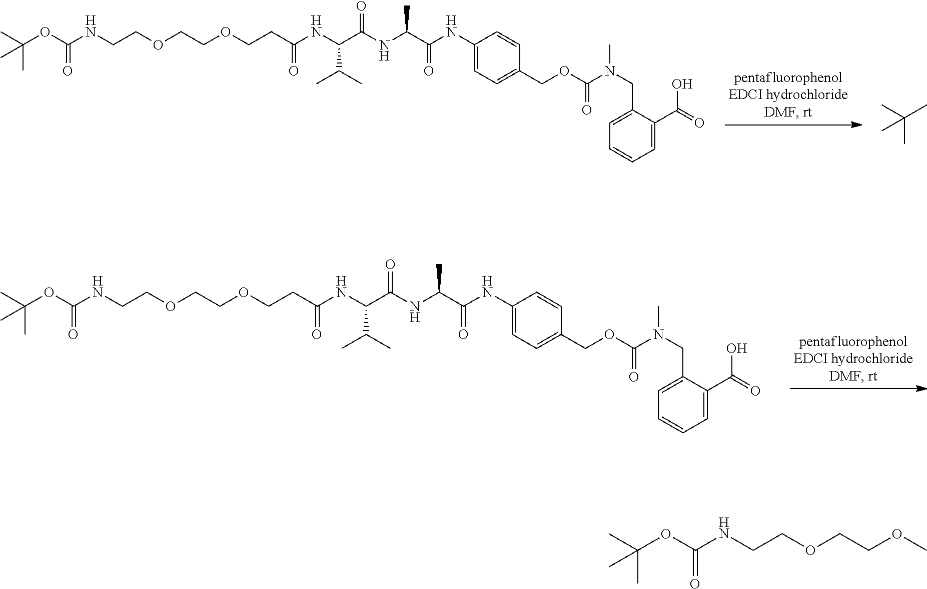

To a solution of 2-({[({4-[(2S)-2-[(2S)-2-{3-[2-(2-{[(tert-butoxy)carbonyl]amino}ethoxy)ethoxy]propanamido}-3-methylbutanamido]propanamido]phenyl}methoxy)carbonyl](methyl)amino}methyl)benzoic acid (150 mg, 0.202 mmol)) and pentafluorophenol (CAS No. 771-61-9, 81 mg, 0.44 mmol) in DMF (2.0 mL) at room temperature was added EDCI hydrochloride (80 mg, 0.417 mmol). The reaction mixture was stirred at room temperature for 35 min. The reaction mixture was concentrated under reduced pressure. The residue was purified by flash chromatography (70% to 100% EtOAc/heptane) to give 2,3,4,5,6-pentafluorophenyl 2-({[({4-[(2S)-2-[(2S)-2-{3-[2-(2-{[(tert-butoxy)carbonyl]amino}ethoxy)ethoxy]propanamido}-3-methylbutanamido]propanamido]phenyl}methoxy)carbonyl](methyl)amino}methyl)benzoate (158 mg) as a colorless oil.

ESI-MS (m/z): 910.53 [M+H]⁺.

¹H NMR (500 MHz, CHLOROFORM-d) δ ppm 0.82-1.13 (m, 6H), 1.36-1.61 (m, 12H), 2.22 (br s, 1H), 2.47-2.77 (m, 2H), 2.99 (br s, 3H), 3.21-3.40 (m, 2H), 3.44-3.65 (m, 6H), 3.65-3.84 (m, 2H), 4.25 (br s, 1H), 4.60-4.84 (m, 1H), 4.88-4.98 (m, 2H), 5.05-5.19 (m, 2H), 6.14 (br s, 1H), 6.81-7.04 (m, 1H), 7.17 (br s, 1H), 7.27-7.50 (m, 3H), 7.51-7.76 (m, 3H), 8.20-8.39 (m, 1H), 8.54-8.71 (m, 1H), 8.88-9.13 (m, 1H).

### 8.3 Synthesis of {4-[(2S)-2-[(2S)-2-{3-[2-(2-{[(tert-butoxy)carbonyl]amino}ethoxy)ethoxy]propanamido}-3-methylbutanamido]propanamido]phenyl}methyl N-({2-[(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,77}]dotetraconta-5,7,9,11,15,19,21,23,25-nonaene-13-carbonyl]phenyl}methyl)-N-methylcarbamate

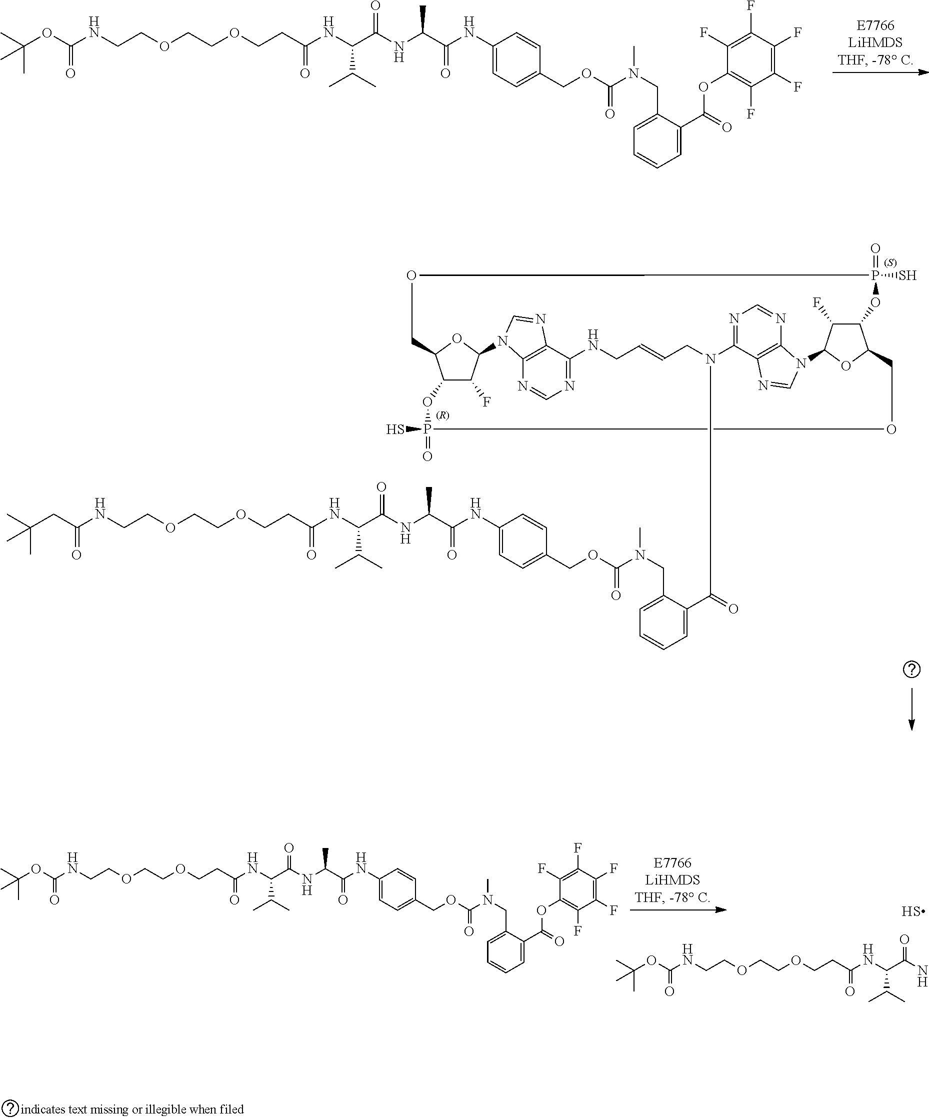

To a white suspension of Compound 1 (134 mg, 0.157 mmol) in THF (13 mL) at room temperature was added LiHMDS (1.5 mL, 1.95 mmol) dropwise for 2 min. The reaction mixture was stirred at room temperature for 30 min. The mixture was cooled to −78° C. To the mixture was added 2,3,4,5,6-pentafluorophenyl 2-({[({4-[(2S)-2-[(2S)-2-{3-[2-(2-{[(tert-butoxy)carbonyl]amino}ethoxy)ethoxy]propanamido}-3-methylbutanamido]propanamido]phenyl}methoxy)carbonyl](methyl)amino}methyl)benzoate (158 mg, 0.174 mmol) in THF (1.0 mL) with THF rinse (1.0 mL×2). The reaction mixture was stirred at −78° C. for 5 min. Then the cooling bath was removed, and the reaction mixture was allowed to warm to room temperature. After stirring for 1 h, the reaction mixture was cooled to −78° C. (dry ice-EtOH bath). The reaction was quenched with AcOH (0.225 mL, 3.929 mmol) at −78° C. Then it was stirred at 0° C. for 1 h. The reaction mixture was allowed to warm to room temperature. The mixture was concentrated under reduced pressure. The residue was purified by reverse-phase flash chromatography on ODS (10% to 50% MeCN/H₂O containing 0.1% HCO₂H) to give {4-[(2S)-2-[(2S)-2-{3-[2-(2-{[(tert-butoxy)carbonyl]amino}ethoxy)ethoxy]propanamido}-3-methylbutanamido]propanamido]phenyl}methyl N-({2-[(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.13,36 128,31.04,8.07,12.019,24.023,27]dotetraconta-5,7,9,11,15,19,21,23,25-nonaene-13-carbonyl]phenyl}methyl)-N-methylcarbamate (112 mg) as a white solid.

ESI-MS (m/z): 1472.57 [M+H]⁺.

### 8.4 Synthesis {4-[(2S)-2-[(2S)-2-[3-(2-{2-[2-(2,5-dioxo-2,5-dihydro-1H-pyrrol-1-yl)acetamido]ethoxy}ethoxy)propanamido]-3-methylbutanamido]propanamido]phenyl}methyl N-({2-[(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carbonyl]phenyl}methyl)-N-methylcarbamate (LP54)

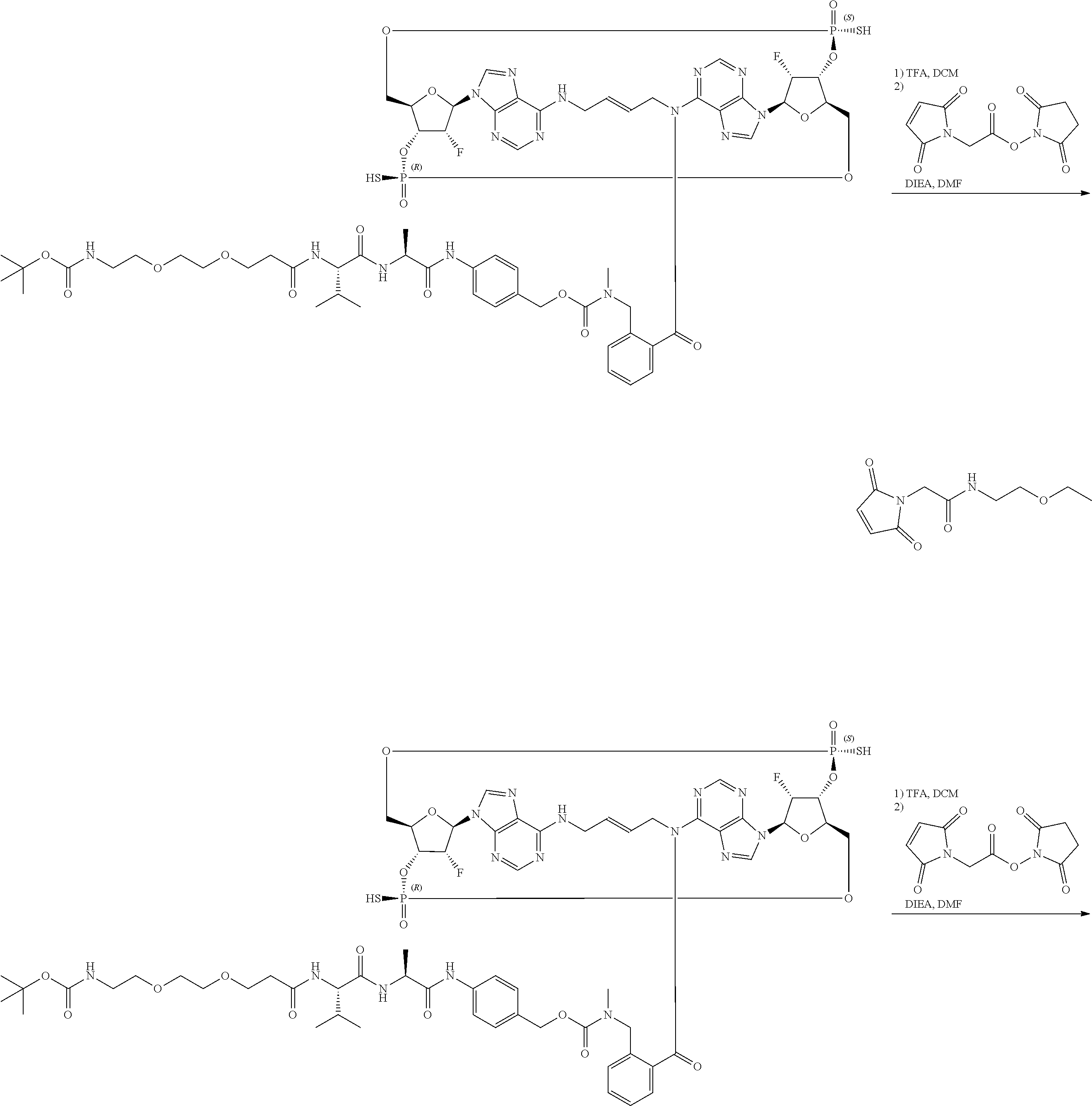

To a suspension of {4-[(2S)-2-[(2S)-2-{3-[2-(2-{[(tert-butoxy)carbonyl]amino}ethoxy)ethoxy]propanamido}-3-methylbutanamido]propanamido]phenyl}methyl N-({2-[(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.13,36 128,31.04,8.07,12.019,24.023,27]dotetraconta-5,7,9,11,15,19,21,23,25-nonaene-13-carbonyl]phenyl}methyl)-N-methylcarbamate (52.8 mg, 0.036 mmol) in DCM (5.0 mL) at 0° C. was added TFA (0.5 mL, 6.49 mmol). The mixture was stirred at 0° C. for 10 min, then the reaction mixture was stirred at room temperature for 38 min. Solvent was removed azeotropically with toluene (three times). The residue was dried under high vacuum. To the residue was added DMF (2.0 mL). To the solution were added DIEA (62.4 μL, 0.358 mmol) and 2,5-dioxopyrrolidin-1-yl 2-(2,5-dioxo-2,5-dihydro-1H-pyrrol-1-yl)acetate (27.1 mg, 0.108 mmol) at 0° C. The mixture was stirred at 0° C. for 25 min. The reaction was quenched by the addition of formic acid (13.8 μL, 0.36 mmol) at 0° C. The reaction mixture was concentrated under reduced pressure. The residue was purified by reverse-phase flash chromatography on ODS (10% to 50% MeCN/H₂O containing 0.1% HCO₂H) to give {4-[(2S)-2-[(2S)-2-[3-(2-{2-[2-(2,5-dioxo-2,5-dihydro-1H-pyrrol-1-yl)acetamido]ethoxy}ethoxy)propanamido]-3-methylbutanamido]propanamido]phenyl}methyl N-({2-[(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carbonyl]phenyl}methyl)-N-methylcarbamate (31.6 mg) as a white solid.

ESI-MS (m/z): 1509.40 M+H]⁺.

¹H NMR (500 MHz, DMSO-d₆) δ ppm 0.83 (br d, J=6.72 Hz, 3H), 0.87 (br d, J=6.11 Hz, 3H), 1.27-1.34 (m, 3H), 1.91-2.01 (m, 1H), 2.40 (br t, J=6.11 Hz, 1H), 2.46 (t, J=6.72 Hz, 1H), 2.73-2.88 (m, 3H), 3.19 (q, J=5.91 Hz, 2H), 3.39 (br t, J=5.81 Hz, 2H), 3.47-3.49 (m, 4H), 3.58-3.83 (m, 4H), 4.02 (s, 2H), 4.16-4.34 (m, 5H), 4.34-5.35 (m, 11H), 5.37-6.00 (m, 3H), 6.35-6.46 (m, 2H), 6.86-7.06 (m, 3H), 7.08 (s, 2H), 7.14-7.28 (m, 2H), 7.36 (br d, J=7.95 Hz, 1H), 7.47-7.57 (m, 1H), 7.57-7.64 (m, 1H), 7.89 (br d, J=8.56 Hz, 1H), 8.14-8.25 (m, 2H), 8.29-8.56 (m, 2H), 8.69 (br s, 1H), 9.95 (br d, J=16.51 Hz, 1H).

### Example 9. Synthesis of {4-[(2S)-2-[(2S)-2-(3-{2-[2-(2,5-dioxo-2,5-dihydro-1H-pyrrol-1-yl)ethoxy]ethoxy}propanamido)-3-methylbutanamido]propanamido]phenyl}methyl N-({2-[(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carbonyl]phenyl}methyl)-N-methylcarbamate (LP55)

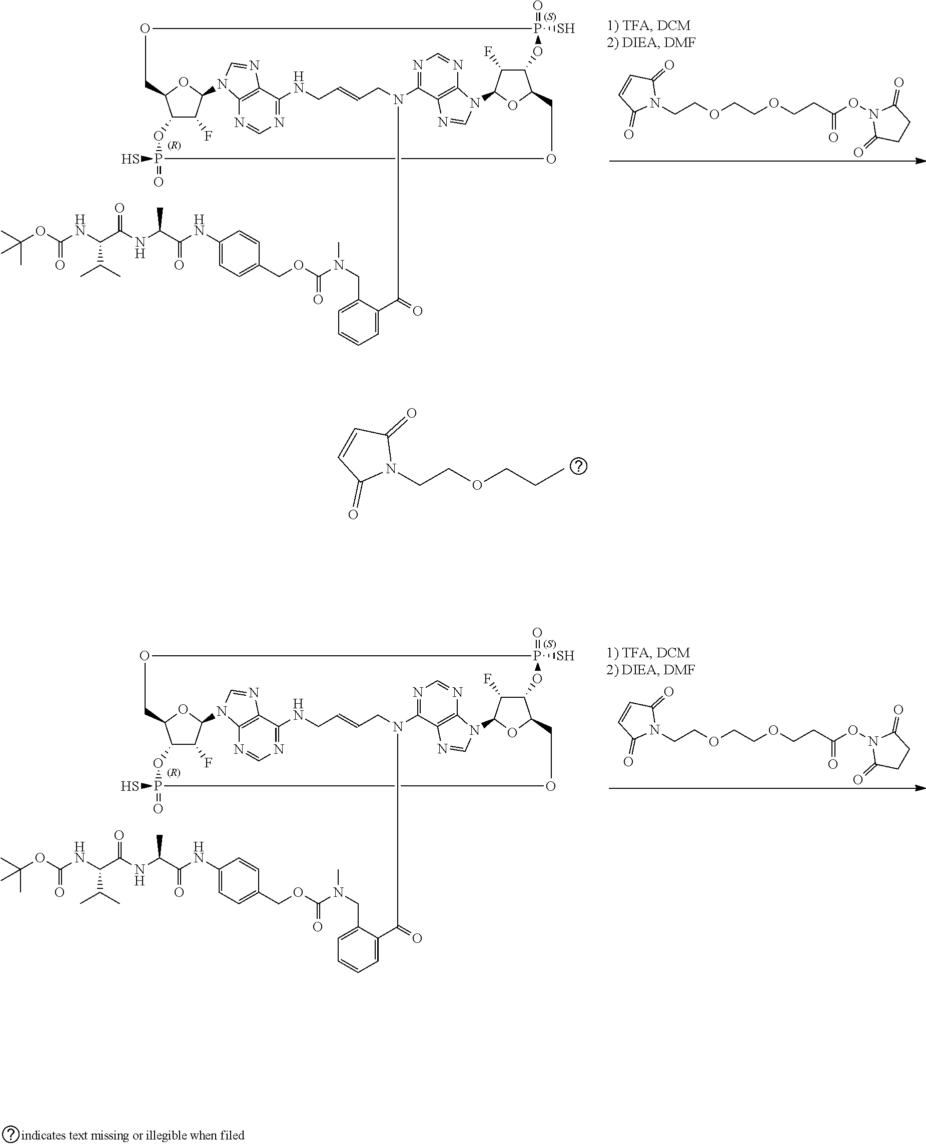

To a suspension of {4-[(2S)-2-[(2S)-2-{[(tert-butoxy)carbonyl]amino}-3-methylbutanamido]propanamido]phenyl}methyl N-({2-[(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4. 1^{3,36}.1^{28,31}.0^{4,8}.0^{7,12}.0^{19,24}.0^{23,27}]dotetraconta-5,7,9,11,15,19,21,23,25-nonaene-13-carbonyl]phenyl}methyl)-N-methylcarbamate (31.0 mg, 0.024 mmol) in DCM (1.5 ml) at 0° C. was added TFA (0.2 mL). The mixture was stirred at 0° C. for 1 min, then the reaction mixture was stirred at room temperature for 58 min. Solvent was removed azeotropically with toluene (three times). The residue was dried under high vacuum. The residue was dissolved in DMF (1.0 mL). To the solution were added DIEA (41.0 μL, 0.235 mmol) and 2,5-dioxopyrrolidin-1-yl 3-{2-[2-(2,5-dioxo-2,5-dihydro-1H-pyrrol-1-yl)ethoxy]ethoxy}propanoate (25.4 mg, 0.072 mmol) at 0′° C. The mixture was stied at 0 IC for 80 m. The reaction was quenched by the addition of formic acid (9.96 μL, 0.26 mmol) at 0° C. The mixture was concentrated under reduced pressure. The residue was purified by reverse-phase flash chromatography on ODS (10% to 50% MeCN/H₂O containing 0.1% formic acid) to give {4-[(2S)-2-[(2S)-2-(3-{2-[2-(2,5-dioxo-2,5-dihydro-1H-pyrrol-1-yl)ethoxy]ethoxy}propanamido)-3-methylbutanamido]propanamido]phenyl}methyl N-({2-[(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carbonyl]phenyl}methyl)-N-methylcarbamate (16.0 mg) as a white solid.

ESI-MS (nm/z): 1452.86 [M+H]⁺.

TABLE 26
Exemplary Amino Acid Sequences

| Description | SEQ ID NO | Amino Acid Sequence |
|---|---|---|
| IgG1 CH2 | 240 | GGPSVFLFPPKPKDTLMISRTPEVTCVVVDVSHEDPEVKENWYVD |
| | | GVEVHNAKTKPREEQYNSTYRVVSVLTVLHQDWLNGKEYKCKVSN |
| | | KALPAPIEKTISKAK |
| IgG1 CH3 | 241 | GQPREPQVYTLPPSRDELTKNQVSLTCLVKGFYPSDIAVEWESNG |
| | | QPENNYKTTPPVLDSDGSFFLYSKLTVDKSRWQQGNVFSCSVMHE |
| | | ALHNHYTQKSLSLSPGK |
| IgG1 Fc | 242 | TCPPCPAPELLGGPSVFLFPPKPKDTLMISRTPEVTCVVVDVSHE |
| | | DPEVKFNWYVDGVEVHNAKTKPREEQYNSTYRVVSVLTVLHQDWL |
| | | NGKEYKCKVSNKALPAPIEKTISKAKGQPREPQVYTLPPSRDELT |
| | | KNQVSLTCLVKGFYPSDIAVEWESNGQPENNYKTTPPVLDSDGSF |
| | | FLYSKLTVDKSRWQQGNVFSCSVMHEALHNHYTQKSLSLSPGK |
| STING | 243 | MPHSSLHPSIPCPRGHGAQKAALVLLSACLVTLWGLGEPPEHTLR |
| | | YLVLHLASLQLGLLLNGVCSLAEELRHIHSRYRGSYWRTVRACLG |
| | | CPLRRGALLLLSIYFYYSLPNAVGPPFTWMLALLGLSQALNILLG |
| | | LKGLAPAEISAVCEKGNENVAHGLAWSYYIGYLRLILPELQARIR |
| | | TYNQHYNNLLRGAVSQRLYILLPLDCGVPDNLSMADPNIRFLDKL |
| | | PQQTGDHAGIKDRVYSNSIYELLENGQRAGTCVLEYATPLQTLFA |
| | | MSQYSQAGFSREDRLEQAKLFCRTLEDILADAPESQNNCRLIAYQ |
| | | EPADDSSFSLSQEVLRHLRQEEKEEVTVGSLKTSAVPSTSTMSQE |
| | | PELLISGMEKPLPLRIDES |
| Gly-Gly-Phe-Gly | 244 | GGFG |
| Gly-Phe-Leu-Gly | 245 | GFLG |
| Ala-Leu-Ala-Leu | 246 | ALAL |
| CD73 | 248 | MCPRAARAPATLLLALGAVLWPAAGAWELTILHTNDVHSRLEQTS |
| | | EDSSKCVNASRCMGGVARLFTKVQQIRRAEPNVLLLDAGDQYQGT |
| | | IWFTVYKGAEVAHFMNALRYDAMALGNHEFDNGVEGLIEPLLKEA |
| | | KFPILSANIKAKGPLASQISGLYLPYKVLPVGDEVVGIVGYTSKE |
| | | TPFLSNPGTNLVFEDEITALQPEVDKLKTLNVNKIIALGHSGFEM |
| | | DKLIAQKVRGVDVVVGGHSNTFLYTGNPPSKEVPAGKYPFIVTSD |
| | | DGRKVPVVQAYAFGKYLGYLKIEFDERGNVISSHGNPILLNSSIP |
| | | EDPSIKADINKWRIKLDNYSTQELGKTIVYLDGSSQSCRFRECNM |
| | | GNLICDAMINNNLRHTDEMFWNHVSMCILNGGGIRSPIDERNNGT |
| | | ITWENLAAVLPFGGTFDLVQLKGSTLKKAFEHSVHRYGQSTGEFL |
| | | QVGGIHVVYDLSRKPGDRVVKLDVLCTKCRVPSYDPLKMDEVYKV |
| | | ILPNFLANGGDGFQMIKDELLRHDSGDQDINVVSTYISKMKVIYP |
| | | AVEGRIKFSTGSHCHGSFSLIFLSLWAVIFVLYQ |

1. An internalizing anti-CD73 antibody or internalizing antigen-binding fragment thereof, wherein the antibody or antigen-binding fragment binds specifically to human CD73, and wherein the antibody or antigen-binding fragment comprises three heavy chain complementarity determining regions (HCDR1, HCDR2, and HCDR3) and three light chain complementarity determining regions (LCDR1, LCDR2, and LCDR3), wherein
(i) the HCDR1 comprises the amino acid sequence of SEQ ID NO: 1, the HCDR2 comprises the amino acid sequence of SEQ ID NO: 2, the HCDR3 comprises the amino acid sequence of SEQ ID NO: 3, the LCDR1 comprises the amino acid sequence of SEQ ID NO: 4, the LCDR2 comprises the amino acid sequence of SEQ ID NO: 5, and the LCDR3 comprises the amino acid sequence of SEQ ID NO: 6, as defined by the Kabat numbering system;
(ii) the HCDR1 comprises the amino acid sequence of SEQ ID NO: 7, the HCDR2 comprises the amino acid sequence of SEQ ID NO: 8, the HCDR3 comprises the amino acid sequence of SEQ ID NO: 9, the LCDR1 comprises the amino acid sequence of SEQ ID NO: 10, the LCDR2 comprises the amino acid sequence of GVI, and the LCDR3 comprises the amino acid sequence of SEQ ID NO: 12, as defined by the IMGT numbering system;
(iii) the HCDR1 comprises the amino acid sequence of SEQ ID NO: 13, the HCDR2 comprises the amino acid sequence of SEQ ID NO: 14, the HCDR3 comprises the amino acid sequence of SEQ ID NO: 15, the LCDR1 comprises the amino acid sequence of SEQ ID NO: 16, the LCDR2 comprises the amino acid sequence of SEQ ID NO: 17, and the LCDR3 comprises the amino acid sequence of SEQ ID NO: 18, as defined by the Kabat numbering system;
(iv) the HCDR1 comprises the amino acid sequence of SEQ ID NO: 19, the HCDR2 comprises the amino acid sequence of SEQ ID NO: 20, the HCDR3 comprises the amino acid sequence of SEQ ID NO: 21, the LCDR1 comprises the amino acid sequence of SEQ ID NO: 22, the LCDR2 comprises the amino acid sequence of GAS SEQ 4 NO: 23, and the LCDR3 comprises the amino acid sequence of SEQ ID NO: 24, as defined by the IMGT numbering system;
(v) the HCDR1 comprises the amino acid sequence of SEQ ID NO: 25, the HCDR2 comprises the amino acid sequence of SEQ ID NO: 26, the HCDR3 comprises the amino acid sequence of SEQ ID NO: 27, the LCDR1 comprises the amino acid sequence of SEQ ID NO: 28, the LCDR2 comprises the amino acid sequence of SEQ ID NO: 29, and the LCDR3 comprises the amino acid sequence of SEQ ID NO: 30, as defined by the Kabat numbering system;
(vi) the HCDR1 comprises the amino acid sequence of SEQ ID NO: 31, the HCDR2 comprises the amino acid sequence of SEQ ID NO: 32, the HCDR3 comprises the amino acid sequence of SEQ ID NO: 33, the LCDR1 comprises the amino acid sequence of SEQ ID NO: 34, the LCDR2 comprises the amino acid sequence of YAS, and the LCDR3 comprises the amino acid sequence of SEQ ID NO: 36, as defined by the IMGT numbering system;
(vii) the HCDR1 comprises the amino acid sequence of SEQ ID NO: 37, the HCDR2 comprises the amino acid sequence of SEQ ID NO: 38, the HCDR3 comprises the amino acid sequence of SEQ ID NO: 39, the LCDR1 comprises the amino acid sequence of SEQ ID NO: 40, the LCDR2 comprises the amino acid sequence of SEQ ID NO: 41, and the LCDR3 comprises the amino acid sequence of SEQ ID NO: 42, as defined by the Kabat numbering system; or
(viii) the HCDR1 comprises the amino acid sequence of SEQ ID NO: 43, the HCDR2 comprises the amino acid sequence of SEQ ID NO: 44, the HCDR3 comprises the amino acid sequence of SEQ ID NO: 45, the LCDR1 comprises the amino acid sequence of SEQ ID NO: 46, the LCDR2 comprises the amino acid sequence of DAS, and the LCDR3 comprises the amino acid sequence of SEQ ID NO: 48, as defined by the IMGT numbering system.

2. The anti-CD73 antibody or antigen-binding fragment of claim 1, wherein the antibody or antigen-binding fragment comprises
(i) three HCDRs comprising the amino acid sequences of SEQ ID NO: 1 (HCDR1), SEQ ID NO: 2 (HCDR2), and SEQ ID NO: 3 (HCDR3); and three LCDRs comprising the amino acid sequences of SEQ ID NO: 4 (LCDR1), SEQ ID NO: 5 (LCDR2), and SEQ ID NO: 6 (LCDR3), as defined by the Kabat numbering system;
(ii) three HCDRs comprising the amino acid sequences of SEQ ID NO: 7 (HCDR1), SEQ ID NO: 8 (HCDR2), and SEQ ID NO: 9 (HCDR3); and three LCDRs comprising the amino acid sequences of SEQ ID NO: 10 (LCDR1), GVI (LCDR2), and SEQ ID NO: 12 (LCDR3), as defined by the IMGT numbering system;
(iii) three HCDRs comprising the amino acid sequences of SEQ ID NO: 13 (HCDR1), SEQ ID NO: 14 (HCDR2), and SEQ ID NO: 15 (HCDR3); and three LCDRs comprising the amino acid sequences of SEQ ID NO: 16 (LCDR1), SEQ ID NO: 17 (LCDR2), and SEQ ID NO: 18 (LCDR3), as defined by the Kabat numbering system; or
(iv) three HCDRs comprising the amino acid sequences of SEQ ID NO: 19 (HCDR1), SEQ ID NO: 20 (HCDR2), and SEQ ID NO: 21 (HCDR3); and three LCDRs comprising the amino acid sequences of SEQ ID NO: 22 (LCDR1), GAS (LCDR2), and SEQ ID NO: 24 (LCDR3), as defined by the IMGT numbering system.

3. The anti-CD73 antibody or antigen-binding fragment of claim 1, wherein the antibody or antigen-binding fragment comprises
(i) a heavy chain variable region comprising the amino acid sequence of SEQ ID NO: 49, and a light chain variable region comprising the amino acid sequence of SEQ ID NO: 50 or 51;
(ii) a heavy chain variable region comprising the amino acid sequence of SEQ ID NO: 52, and a light chain variable region comprising the amino acid sequence of SEQ ID NO: 53 or 54;
(iii) a heavy chain variable region comprising the amino acid sequence of SEQ ID NO: 55, and a light chain variable region comprising the amino acid sequence of SEQ ID NO: 56 or 57; or
(iv) a heavy chain variable region comprising the amino acid sequence of SEQ ID NO: 58, and a light chain variable region comprising the amino acid sequence of SEQ ID NO: 59 or 60.

4. The anti-CD73 antibody or antigen-binding fragment of claim 3, wherein the antibody or antigen-binding fragment comprises
(i) a heavy chain variable region comprising the amino acid sequence of SEQ ID NO: 49, and a light chain variable region comprising the amino acid sequence of SEQ ID NO: 50;
(ii) a heavy chain variable region comprising the amino acid sequence of SEQ ID NO: 49, and a light chain variable region comprising the amino acid sequence of SEQ ID NO: 51;
(iii) a heavy chain variable region comprising the amino acid sequence of SEQ ID NO: 52, and a light chain variable region comprising the amino acid sequence of SEQ ID NO: 53; or
(iv) a heavy chain variable region comprising the amino acid sequence of SEQ ID NO: 52, and a light chain variable region comprising the amino acid sequence of SEQ ID NO: 54.

5. The anti-CD73 antibody or antigen-binding fragment of claim 1, wherein the antibody or antigen-binding fragment comprises a human IgG heavy chain constant region.

6. The anti-CD73 antibody or antigen-binding fragment of claim 5, wherein the antibody or antigen-binding fragment comprises an IgG1 heavy chain constant region comprising an Fc domain mutated to reduce binding to an Fcγ receptor (FcγR) as compared to an IgG1 heavy chain constant region comprising a wild type IgG1 Fc domain.

7. The anti-CD73 antibody or antigen-binding fragment of claim 6, wherein the mutated IgG1 Fc domain comprises the mutations L234A, L235A, P238S, H268Q, and K274Q.

8. The anti-CD73 antibody or antigen-binding fragment of claim 1, wherein the antibody or antigen-binding fragment comprises a human Ig kappa light chain constant region

9. The anti-CD73 antibody or antigen-binding fragment of claim 1, wherein the antibody or antigen-binding fragment comprises:
(i) a heavy chain comprising the amino acid sequence of SEQ ID NO: 61, 62, 63, or 64 and a light chain comprising the amino acid sequence of SEQ ID NO: 65 or 66;
(ii) a heavy chain comprising the amino acid sequence of SEQ ID NO: 67, 68, 69, or 70 and a light chain comprising the amino acid sequence of SEQ ID NO: 71 or 72;
(iii) a heavy chain comprising the amino acid sequence of SEQ ID NO: 73, 74, 75, or 76 and a light chain comprising the amino acid sequence of SEQ ID NO: 77 or 78;
(iv) a heavy chain comprising the amino acid sequence of SEQ ID NO: 79, 80, 81, or 82 and a light chain comprising the amino acid sequence of SEQ ID NO: 83 or 84;
(v) a heavy chain comprising the amino acid sequence of SEQ ID NO: 111, 112, 113, or 114 and a light chain comprising the amino acid sequence of SEQ ID NO: 65 or 66;
(vi) a heavy chain comprising the amino acid sequence of SEQ ID NO: 115, 116, 117, or 118 and a light chain comprising the amino acid sequence of SEQ ID NO: 71 or 72;
(vii) a heavy chain comprising the amino acid sequence of SEQ ID NO: 119, 120, 121, or 122 and a light chain comprising the amino acid sequence of SEQ ID NO: 77 or 78; or
(viii) a heavy chain comprising the amino acid sequence of SEQ ID NO: 123, 124, 125, or 126 and a light chain comprising the amino acid sequence of SEQ ID NO: 83 or 84.

10. The anti-CD73 antibody or antigen-binding fragment of claim 9, wherein the antibody or antigen-binding fragment comprises:
(i) a heavy chain comprising the amino acid sequence of SEQ ID NO: 63 and a light chain comprising the amino acid sequence of SEQ ID NO: 65;
(ii) a heavy chain comprising the amino acid sequence of SEQ ID NO: 63 and a light chain comprising the amino acid sequence of SEQ ID NO: 66;
(iii) a heavy chain comprising the amino acid sequence of SEQ ID NO: 64 and a light chain comprising the amino acid sequence of SEQ ID NO: 65;
(iv) a heavy chain comprising the amino acid sequence of SEQ ID NO: 64 and a light chain comprising the amino acid sequence of SEQ ID NO: 66;
(v) a heavy chain comprising the amino acid sequence of SEQ ID NO: 69 and a light chain comprising the amino acid sequence of SEQ ID NO: 71;
(vi) a heavy chain comprising the amino acid sequence of SEQ ID NO: 69 and a light chain comprising the amino acid sequence of SEQ ID NO: 72;
(vii) a heavy chain comprising the amino acid sequence of SEQ ID NO: 70 and a light chain comprising the amino acid sequence of SEQ ID NO: 71; or
(viii) a heavy chain comprising the amino acid sequence of SEQ ID NO: 70 and a light chain comprising the amino acid sequence of SEQ ID NO: 72.

11. The anti-CD73 antibody or antigen-binding fragment of claim 1, wherein the antibody or antigen-binding fragment comprises part of a bispecific or multi-specific binding construct.

12. The anti-CD73 antibody or antigen-binding fragment of claim 1, wherein the antibody or antigen-binding fragment is linked to a therapeutic agent or detectable agent.

13. An antibody-drug conjugate of Formula (I):
Ab-(L-D)ₚ (I)
wherein Ab is an anti-CD73 antibody or antigen-binding fragment thereof of claim 1;
D is a therapeutic agent;
L is a cleavable linker that covalently attaches Ab to D; and
p is an integer from 1 to 20.

14. The antibody-drug conjugate of claim 13, wherein the therapeutic agent comprises a STING agonist.

15. The antibody-drug conjugate of claim 14, wherein the therapeutic agent comprises a compound selected from: 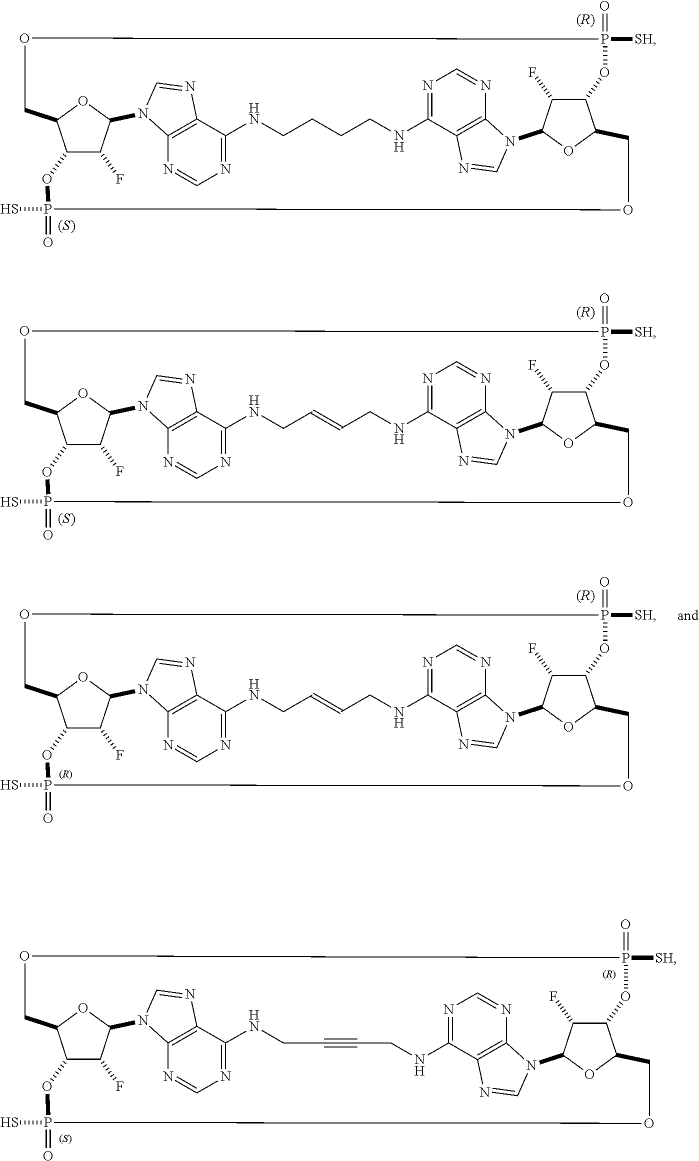
or a salt thereof.

16.-42. (canceled)

43. The antibody-drug conjugate of claim 13, wherein the L-D comprises LP2:

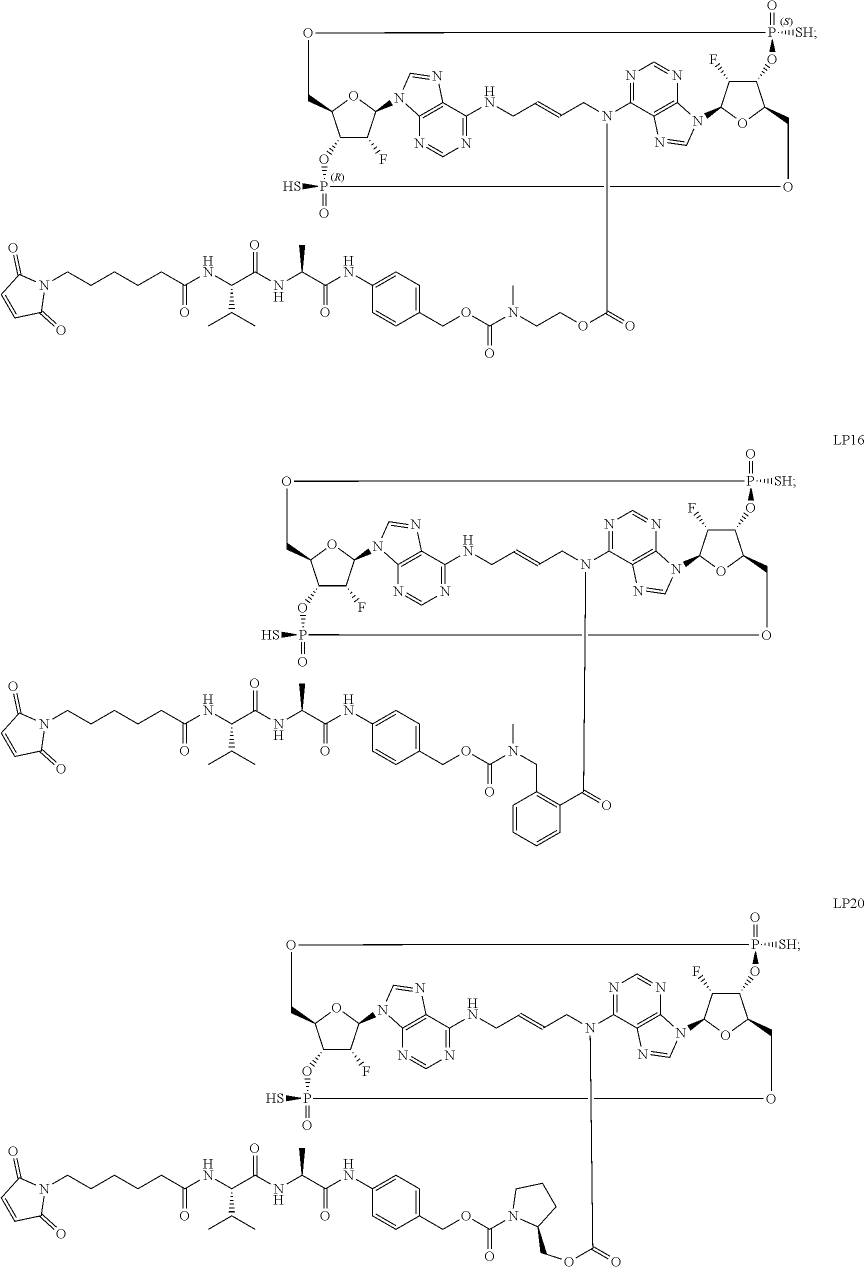

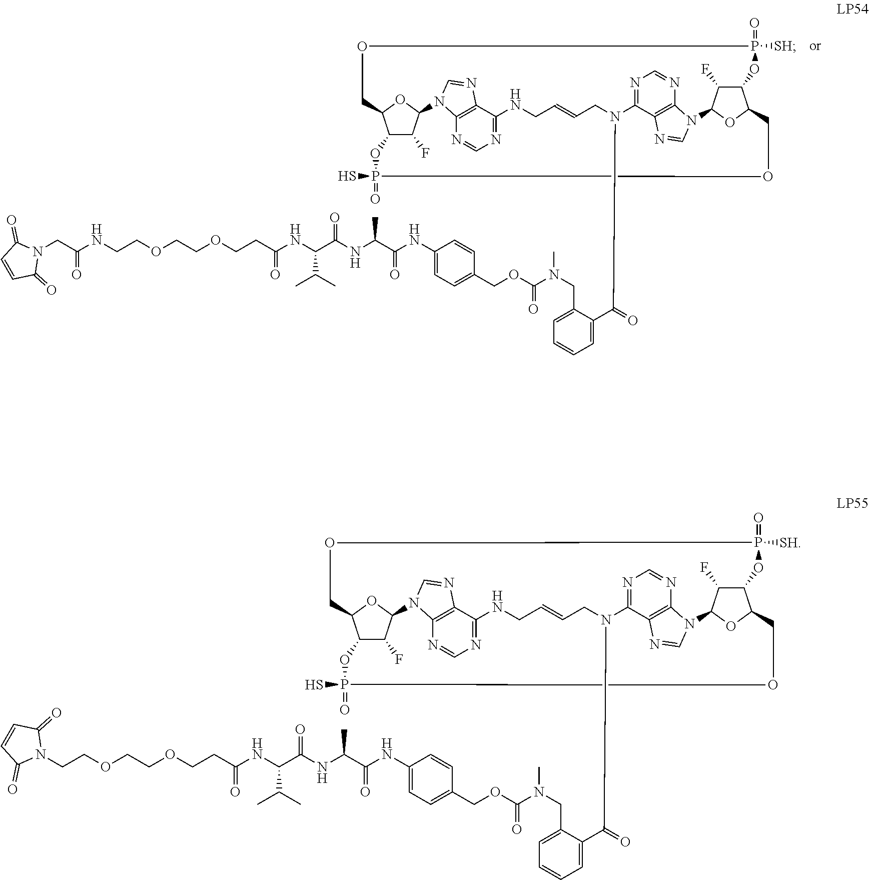 [SMILES: CC(C)[C@H](NC(=O)CCOCCOCCNC(=O)CN1C(=O)C=CC1=O)C(=O)N[C@@H](C)C(=O)Nc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1 ; CC(C)[C@H](NC(=O)CCOCCOCCN1C(=O)C=CC1=O)C(=O)N[C@@H](C)C(=O)Nc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

44.-78. (canceled)

79. A pharmaceutical composition comprising the antibody or antigen-binding fragment of claim 1 and a pharmaceutically acceptable carrier.

80. A method of treating a patient having or at risk of having a cancer that expresses CD73, comprising administering to the patient a therapeutically effective amount of the antibody or antigen-binding fragment of claim 1.

81. The method of claim 80, wherein the CD73-expressing cancer is melanoma, diffuse large B-cell lymphoma, T-cell lymphoma, breast cancer, ovarian cancer, head and neck cancer, head and neck squamous carcinoma, non-small-cell lung cancer (NSCLC), glioblastoma, thyroid carcinoma, pancreatic cancer, pancreatic ductal adenocarcinoma (PDAC), esophageal cancer, cervical cancer, gastric cancer, gallbladder cancer, or colorectal cancer.

82. A method of stimulating an anti-tumor immune response in the tumor microenvironment of a patient having a cancer that expresses CD73, comprising administering to the patient a therapeutically effective amount of the antibody or antigen-binding fragment of claim 1.

83. A method of reducing or inhibiting growth of a CD73-expressing tumor, comprising administering a therapeutically effective amount of an antibody or antigen-binding fragment of claim 1.

84.-87. (canceled)

88. A nucleic acid encoding the antibody or antigen-binding fragment of claim 1.

89. A cell or cell population comprising the nucleic acid of claim 88.

90. A method of producing an antibody or antigen-binding fragment of claim 1, comprising culturing a cell or cell population comprising a nucleic acid encoding the antibody or antigen-binding fragment under conditions sufficient to produce the antibody or antigen-binding fragment.

91. A method of producing the antibody-drug conjugate of claim 13, comprising reacting the antibody or antigen-binding fragment with a cleavable linker joined to D under conditions that allow conjugation, wherein D comprises a compound selected from: 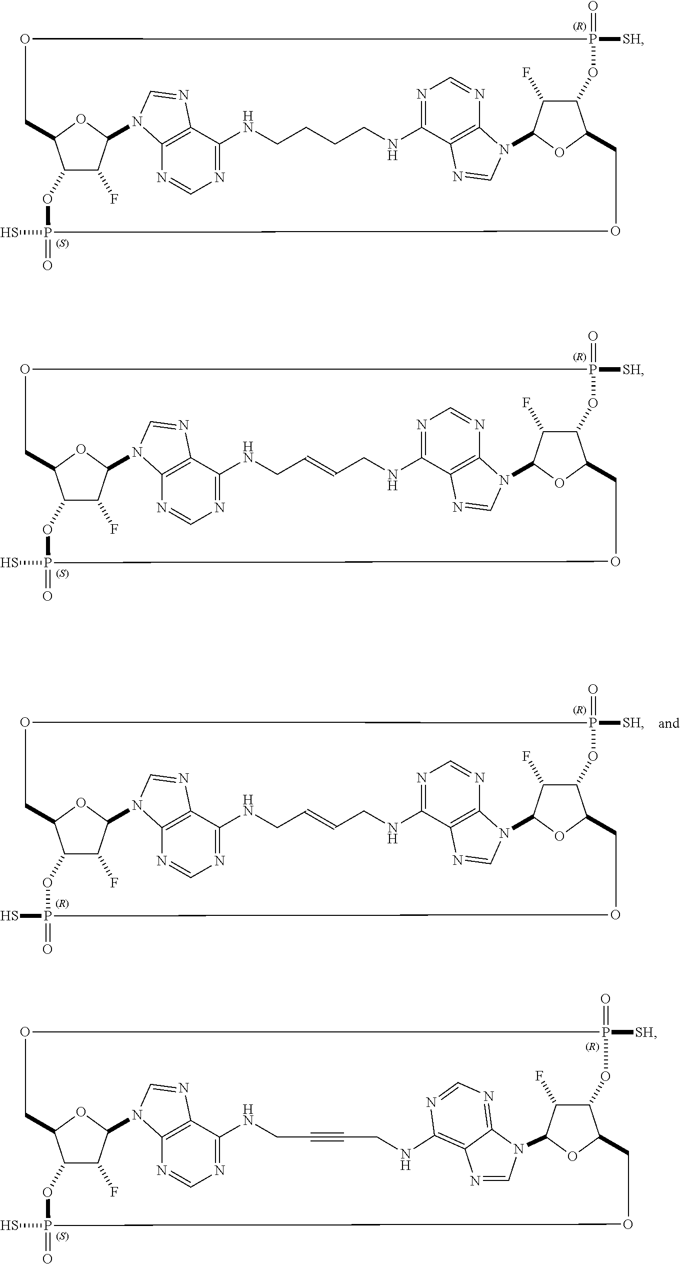
or a salt thereof.

92. (canceled)

93. A pharmaceutical composition comprising the antibody-drug conjugate of claim 13 and a pharmaceutically acceptable carrier.

94. A method of treating a patient having or at risk of having a cancer that expresses CD73, comprising administering to the patient a therapeutically effective amount of the antibody-drug conjugate of claim 13.
